(19) United States
(12) Patent Application Publication
(10) Pub. No.: US 2004/0096951 A1
(43) Pub. Date: May 20, 2004

(54) Crystal structures of retaining glycosytransferases

(76) Inventors: Stephen G. Withers, Vancouver (CA); Warren W. Wakarchuk, Gloucester (CA); Natalie C.J. Strynadka, Vancouver (CA); Manuela Dieckelmann, Brisbane (AU); Hoa Ly, Kitchener Ontario (CA); Karina Persson, Vancouver (CA)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/450,802

(22) PCT Filed: Dec. 14, 2001
(86) PCT No.: PCT/CA01/01793

(51) Int. Cl. C12P 19/38; C12N 9/10; C08B 37/00

(52) U.S. Cl. 435/193; 435/87; 536/53

(57) ABSTRACT

The present invention relates to a crystal comprising the ligand binding pocket of a glycosyltransferase enzyme and optionally a donor molecule or analogue thereof and/or an acceptor molecule or analogue thereof. The present invention also relates to the use of such a crystal to identify ligands capable of modulating glycosyltransferase activity, and the use of such ligands in therapeutic applications.

A portion of the disclosure of this patent document contains material that is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates to crystal structure. In particular a crystal comprising a ligand-binding pocket (LBP) of a glycosyltransferase, optionally having a ligand associated therewith. The invention also relates to a crystal of a retaining glycosyltransferase and parts thereof. In particular, the present invention relates to a crystal comprising a ligand binding pocket of a retaining galactosyltransferase optionally in association with a ligand, for example a donor and/or an acceptor molecule or analogue thereof. The crystals may be useful for modeling and/or synthesizing mimetics of a ligand binding pocket, or ligands that associate with the binding pocket. Such mimetics or ligands may be capable of acting as modulators of glycosyltransferase activity, and they may be useful for treating, inhibiting, or preventing diseases associated with or modulated by glycosyltransferases. The structures may be used to determine retaining glycosyltransferase homologs and information about the secondary and tertiary structures of polypeptides which are as yet structurally uncharacterized.

### BACKGROUND

Oligosaccharides are essential to a wide variety of biological functions many of which are crucial for the development, growth, function and survival of an organism (Varki, 1993 Glycobiology 3, 97-130). Lipooligosaccharide (LOS) is the major glycolipid found on the cell surface of gram-negative mucosal pathogens such as Neisseria, Haemophilus, Moraxella, Bordetella and Campylobacter. The LOS structure is made up of a lipid A moiety, 2-keto-3-deoxyoctulosonic acid (KDO) and various terminal oligosaccharides. Bacterial LOS structures can be antigenically and structurally similar to human glycolipids, and thus may camouflage the bacterial surface from recognition by the human immune system. For example, the terminal structures of N. meningitidis and N. gonorrhoea LOS mimic human lacto-N-neotetraose, sialylacto-N-neotetraose and the P^{k} k blood group glycolipid.

There have been numerous studies on the genes involved in making the oligosaccharide portion of the LOS structure (Wakarchuk et al, 1996 J. Biol. Chem. 271:19166-19173, Moran et al, 1996 FEMS Immunology and Medical Microbiology 16, 105-115, Kahler & Stephens, 1998 Crit. Rev. Microbiol. 24, 281-334 Biochemical Journal 329, 929-939). An essential aspect of this biosynthesis involves the linking of saccharides with the aid of glycosyltransferases (for classification of the distinct families see Campbell et al., 1997 Biochemical Journal 326, 929-939). These enzymes transfer a sugar, mainly from a nucleotide diphospho-sugar, but also from sugar phosphates, to specific acceptor molecules. α-1, 4-Galactosyltransferase from N. meningitidis (Family 8 in the scheme of Campell et al., 1997 as above), adds an α-galactose from UDPgal (the donor) to a terminal lactose (the acceptor) of the LOS structure creating the P^{k} blood group glycolipid mimic (FIG. 1a). Interest in understanding this and other bacterial transferases stems, at least in part, from the notion that inhibitors that block the essential formation of LOS biosynthesis in pathogenic bacteria may prove to be useful new antibiotics (Takayama et al, 1999 Bioorganic and Medicinal Chemistry 7, 401-409).

Based on the relative anomeric stereochemistries of the substrate and product in the reaction catalyzed, glycosyltransferases can be classified mechanistically as either inverting or retaining, (as is also done with the well-studied glycosidase family; Sinnott, 1990, Chem. Rev., 90, 1171-1202; Davies et al., 1997 in Comprehensive Biological Catalysis (Sinnott, M. L., ed) Vol. 1, pp. 119-208, Academic Press, London; Zechel & Withers, 2000 Acc. Chem. Res. 33, 11-18). While this has led to the tacit assumption that similar mechanisms are employed by glycosidases and glycosyltransferases in carrying out their functions, very little has been experimentally verified about the mechanism of the latter.

By simple analogy with the glycosidase counterpart, inverting glycosyltransferases are believed to follow a direct displacement mechanism with a general base to assist in deprotonating the reactive hydroxyl of the acceptor molecule and possibly a general acid to aid in cleavage of the exocyclic C₁—O bond, though a bound metal ion may fulfil this role (FIG. 1b). In the case of retaining transferases, catalysis is believed to proceed via a double displacement mechanism involving the formation and subsequent breakdown of a covalent glycosyl-enzyme intermediate (FIG. 1c). In such a scheme, a nucleophile is required for attack on the anomeric centre of the donor sugar to form the glycosyl-enzyme species. Once again, either an acid catalyst or a metal ion will be required to provide general acid assistance to the cleavage of the exocyclic C₁—O bond (in the first step) and a general base will be needed to deprotonate the reactive hydroxyl of the acceptor molecule (in the second step).

To date the x-ray crystal structures of only four glycosyltransferases have been determined and all catalyze inverting reactions; β-glucosyltransferase from phage T4, (Vrielink et al.,1994 EMBO J. 13, 3413-3422), SpsA from Bacillus subtilis (Charnock and Davies, 1999 Biochemistry. 38, 6380-6385.), bovine β-1,4-galactosyltransferase (Gastinel et al., 1999) and MurG from E. coli (Ha et al, 2000). With the exception of MurG, these structures have been determined in complex with UDP or the donor UDP-sugar. However, electron density for the sugar moiety has not been observed in any of these experiments.

Despite the fact that the biosynthesis of polysaccharides is of fundamental biological importance, and that a large number of glycosyltransferases have already been described, to date no crystal structures have been provided for retaining glycosyltransferase enzymes, and no crystal structures of any glycosyltransferase enzymes have been provided which give any structural data for the donor and/or acceptor molecule.

Such crystal structures would be invaluable in understanding the catalytic mechanism of the enzymes and in the rational design of inhibitors.

### SUMMARY OF THE INVENTION

The present invention is based on the finding that it is possible to crystallize a retaining glycosyltransferase, both alone and in combination with a selection of different ligands. More particularly, Applicants have crystallized a retaining glycosyltransferase in complex with a metal cofactor, a donor molecule, and in the presence or absence of an acceptor molecule, and have solved the three-dimensional structure of the enzyme. Solving the crystal structure has enabled the determination of key structural features of retaining glycosyltransferases, particularly the shape of ligand binding pockets (also referred to herein as “LBP”), or parts thereof, that associate with a metal cofactor, donor molecule, and/or acceptor molecule. The crystal structure has also enabled the determination of key structural features in donor molecules and acceptor molecules.

Binding pockets are of significant utility in drug discovery. The association of natural ligands and substrates with the binding pockets of their corresponding glycosyltransferases is the basis of many biological mechanisms. In addition, many drugs exert their effects through association with the binding pockets of glycosyltransferases. The associations may occur with all or any parts of a binding pocket An understanding of these associations will lead to the design and optimization of drugs having more favorable associations with their target glycosyltransferase and thus provide improved biological effects. Therefore, information about the shape and structure of glycosyltransferases and their ligand-binding pockets is invaluable in designing potential modulators of glycosyltransferases for use in treating diseases and conditions associated with or modulated by the glycosyltransferases.

Therefore, broadly stated the present invention relates to the secondary, tertiary, and/or quanternary structures of glycosyltransferases, and parts thereof. The glycosyltransferase structure may be the structure the enzyme forms when it is associated with one or more ligands (e.g. an acceptor molecule, a donor molecule, or components thereof). The invention also contemplates a glycosyltransferase structure comprising a the secondary, tertiary, and/or quanternary structure of a glycosyltransferase in association with a ligand. The defined boundaries and properties of the structures and any of the ligands bound to it are pertinent to methods for determining the secondary, tertiary, and/or quanternary structures of polypeptides with unknown structure, and to methods that identify modulators of glycosyltansferases. These modulators are potentially useful as therapeutics for diseases associated with or modulated by glycosytransferases.

In an embodiment, the invention provides a crystal of a polypeptide corresponding to a retaining glycosyltransferase, or a part thereof (e.g. ligand binding pocket). The invention preferably contemplates the crystal a retaining glycosyltransferase forms when it is complexed with a ligand, including a donor molecule or analogue thereof, an acceptor molecule or analogue thereof, a metal cofactor, and/or heavy metal atom. The crystal form may also comprise one or more ligands (e.g. donor molecule or acceptor molecule).

A glycosyltransferase structure of the invention may be characterized by the following:

(a) a ligand binding pocket comprising a core β-sheet containing 7 strands (β33, β2, β1, β4, β6, β8 in FIG. 3) all of which are parallel with the exception of β7; the core β-sheet being further characterized by a nucleotide binding motif composed of four parallel strands sandwiched between helices A and B on one side and helices C and D on the other as illustrated in FIG. 3;

(b) an antiparallel β-ribbon formed by two strands (β5 and β9) lying almost perpendicular to the core β-sheet, and a substrate binding cleft that lies along the base of the core β-sheet; or

(c) a C-terminal domain mediating membrane attachment comprising helix M and Helix N in FIG. 3 forming a small pedestal that packs perpendicular to helices A and B of the nucleotide binding motif and to the β-ribbon as shown in FIG. 3.

The present invention also contemplates molecules or molecular complexes that comprise all or parts of either one or more ligand binding pockets, or homologues of these ligand binding pockets that have similar three-dimensional shapes.

According to an aspect of the invention there is provided a crystal comprising a ligand binding pocket of a retaining glycosyltransferase.

A ligand binding pocket may include one or more of the binding domains for a disphosphate group or pyrophosphate of a donor molecule, a nucleotide of a donor molecule, a nitrogeneous heterocyclic base (preferably a pyrimidine base, more preferably uracil) of a donor molecule, a sugar of the nucleotide of a donor molecule, a selected sugar of a donor molecule that is transferred to an acceptor molecule, and/or an acceptor molecule.

The present invention also provides a crystal comprising a ligand binding pocket of a retaining glycosyltransferase and a donor molecule or analogue thereof from which it is possible to derive structural data for the donor molecule or analogue thereof.

The present invention also provides a crystal comprising a ligand binding pocket of a retaining glycosyltransferase and an acceptor molecule or analogue thereof from which it is possible to derive structural data for the acceptor molecule or analogue thereof.

The present invention also provides a crystal comprising the ligand binding pocket of a retaining glycosyltransferase and a metal cofactor.

In an embodiment a crystal of the invention comprises a ligand binding pocket in association with or complexed with a donor molecule or analogue thereof and/or an acceptor molecule or analogue thereof. In another embodiment the ligand binding pocket is associated with or complexed with a donor molecule, a metal cofactor, and an acceptor molecule. These crystals make it possible to derive structural data for a donor molecule or an acceptor molecule, or analogues thereof.

The shape and structure of a ligand binding pocket may be defined by selected atomic contacts in the pocket In an embodiment the ligand binding pocket is defined by one or more atomic interactions or enzyme atomic contacts as set forth in Table 3. Each of the atomic interactions is defined in Table 3 by an atomic contact (more preferably, a specific atom where indicated) on the donor molecule or analogue thereof or acceptor molecule or analogue thereof, and an atomic contact (more preferably a specific atom where indicated) on the glycosyltransferase.

In an embodiment, the ligand binding pocket is an active site binding pocket of a glycosyltransferase. The active site binding pocket refers to the region of a glycosyltransferase where the transfer of a sugar from the donor molecule to the acceptor occurs.

The invention also provides a method for crystallizing a retaining glycosyltransferase, or a part thereof (e.g. ligand binding pocket), or a complex of a retaining glycosyltransferase or a part thereof and a metal cofactor, donor molecule, and/ or acceptor molecule.

The crystal structures of the invention enable a model to be produced for a glycosyltransferase and a part thereof, (e.g. a ligand binding pocket), or complexes of the enzyme or parts thereof The models may provide structural information about the donor and/or acceptor molecule and their interactions with the LBP. Models may also be produced for donor and acceptor molecules.

Therefore the invention also provides a model of a ligand binding pocket designed in accordance with a method of the invention. The invention contemplates a model, crystal, or secondary, tertiary and/or quanternary structure of a glycosyltransferase or ligand binding pocket in association with a ligand or substrate.

The structures and models of the invention provide information about the atomic contacts involved in the interaction between the enzyme and a known ligand which can be used to screen for unknown ligands. Therefore the present invention provides a method of screening for a ligand capable of binding a glycosyltransferase ligand binding domain, comprising the use of a secondary, tertiary or quanternary structure or a model of the invention. For example, the method may comprise the step of contacting a ligand binding domain with a test compound, and determining if the test compound binds to the ligand.

A crystal and/or model of the invention may be used in a method of determining the secondary, tertiary, and/or quanternary structures of a polypeptide with incompletely characterised structure. Thus, a method is provided for determining at least a portion of the secondary, tertiary, and/or quanternary structure of molecules or molecular complexes which contain at least some structurally similar features to a retaining glycosyltransferase. This is achieved by using at least some of the structural coordinates set out in Table 4, 5 or 6.

A structure, crystal and/or model of the invention may be used to design, evaluate, and identity ligands of a glycosyltransferases or homologues thereof. A ligand may be based on the shape and structure of a glycosyltransferase, or a ligand binding pocket or atomic interactions, or atomic contacts thereof. Preferably, a ligand is derived from a ligand binding pocket for a donor molecule or analogue or parts thereof, and/or an acceptor molecule or analogue or parts thereof. The invention also provides modulators that are derived from a DXD motif or the C-terminal binding pocket mediating membrane attachment.

The present invention also contemplates a ligand identified by a method of the invention. A ligand may be a competitive or non-competitive inhibitor of a glycosyltransferase. Preferably, the ligand is a modulator that is capable of modulating the activity of a glycosyltransferase enzyme. Thus, the methods of the invention permit the identification early in the drug development cycle of compounds that have advantageous properties.

In an embodiment, the present invention contemplates a method of identifying a modulator of a glycosyltransferase, or a ligand binding pocket, or a part thereof, comprising the step of applying the structural coordinates of a glycosyltransferase, ligand binding pocket, or atomic interactions, or atomic contacts thereof, to computationally evaluate a test ligand for its ability to associate with the glycosyltransferase, or ligand binding pocket, or part thereof. Use of the structural coordinates of a glycosyltransferase structure or ligand binding pocket, or atomic interactions, or atomic contacts thereof to identify a modulator is also provided.

In an embodiment, the present invention contemplates a method of identifying a modulator of a glycosyltransferase or a ligand binding pocket or binding site thereof, comprising the step of using the structural coordinates of a glycosyltransferase or a ligand binding pocket or binding site thereof, or a model of the invention to computationally evaluate a test compound for its ability to associate with the glycosyltransferase or ligand binding pocket or binding site thereof. Use of the structural coordinates of a glycosyltransferase structure, ligand binding pocket, or binding site thereof, of the invention to identify a ligand or modulator is also provided.

In another embodiment of the invention, a method is provided for identifying a potential modulator of a glycosyltransferase by determining binding interactions between a test compound and atomic contacts of a ligand binding pocket of a glycosyltransferase defined in accordance with the invention comprising:

(a) generating the atomic contacts on a computer screen;

(b) generating test compounds with their spatial structure on the computer screen; and

(c) determining whether the compounds associate or interact with the atomic contacts defining the glycosyltransferase;

(d) identifying test compounds that are potential modulators by their ability to enter into, a selected number of atomic contacts.

Another aspect of the invention provides methods for identifying a potential modulator of a glycosyltransferase function by docking a computer representation of a test compound with a computer representation of a structure of a glycosyltransferase or a ligand binding pocket thereof that is defined as described herein. In an embodiment the method comprises the following steps:

(a) docking a computer representation of a compound from a computer data base with a computer representation of atomic interactions or atomic contacts of a ligand binding pocket of a glycosyltransferase to obtain a complex;

(b) determining a conformation of the complex with a favourable geometric fit and favourable complementary interactions; and

(c) identifying test compounds that best fit the atomic interactions or contacts as potential modulators of the glycosyltransferase.

In another embodiment the method comprises the following steps:

(a) modifying a computer representation of a test compound complexed with a ligand binding pocket of a glycosyltransferase by deleting or adding a chemical group or groups;

(b) determining a conformation of the complex with a favourable geometric fit and favourable complementary interactions; and

(c) identifying a test compound that best fits the ligand binding pocket as a potential modulator of a glycosyltransferase.

In still another embodiment the method comprises the following steps:

(a) generating a computer representation of a test compound complexed with atomic contacts or atomic interactions of a binding pocket of a glycosyltransferase; and

(b) searching for molecules in a data base that are similar to the test compound using a searching computer program, or replacing portions of the test compound with similar chemical structures from a data base using a compound building computer program.

The ligands or compounds identified according to the methods of the invention preferably have structures such that they are able to enter into an association with a ligand binding pocket Selected ligands or compounds may be characterized by their suitability for binding to particular ligand binding pockets. A ligand binding pocket or binding site may be regarded as a type of negative template with which the compounds correlate as positives in the manner described herein and thus the compounds are unambiguously defined. Therefore, it is possible to describe the structure of a compound suitable as a modulator of a glycosyltransferase by accurately defining the atomic interactions to which the compound binds to a ligand binding pocket and deriving the structure of the compound from the spacial structure of the target.

The invention contemplates a method for the design of ligands, in particular modulators, for glycosyltransferase based on the secondary, tertiary or quanternary structure of a donor molecule or acceptor molecule (or part thereof) defined in relation to its spatial association with the three dimensional structure of the glycosyltransferase or a ligand binding pocket thereof. Generally, a method is provided for designing potential inhibitors of a glycosyltransferase comprising the step of using the structural coordinates of a donor molecule or acceptor molecule or part thereof, defined in relation to its spatial association with the secondary, tertiary or quanternary structure or model of a glycosyltransferase or a ligand binding pocket thereof, to generate a compound for associating with the ligand binding pocket of the glycosyltransferase. The following steps are employed in a particular method of the invention: (a) generating a computer representation of a donor molecule or acceptor molecule, or part thereof, defined in relation to its spatial association with the three dimensional structure of a glycosyltransferase or a ligand binding pocket thereof, or defined by the structural coordinates shown in Table 4, 5, or 6; (b) searching for molecules in a data base that are similar to the defined donor molecule or acceptor molecule, or part thereof using a searching computer program, or replacing portions of the compound with similar chemical structures from a database using a compound building computer program.

Therefore, the invention further contemplates classes of ligands, in particular modulators, of a glycosyltransferase based on the secondary, tertiary or quanternary structure of a donor molecule or acceptor molecule, or part thereof, defined in relation to the donor or acceptor molecule's spatial association with a three dimensional structure of a glycosyltransferase.

It will be appreciated that a modulator of a glycosyltransferase may be identified by generating an actual secondary or three-dimensional model of a ligand binding pocket, synthesizing a compound, and examining the components to find whether the required interaction occurs.

A potential ligand or modulator of a glycosyltransferase identified by a method of the present invention may be confirmed as a modulator by synthesizing the compound, and testing its effect on the glycosyltransferase in an assay for that glycosyltransferase's enzymatic activity. Such assays are known in the art. (See for example, Sadler, J. E. et al. Methods Enzymol., 83, 458-514; Schachter, H., et al Methods Enzymol., 179, 351-397; Datti, A., et al Anal.Biochem., 206, 262-266; Palcic, M. M. (1994) Methods Enzymol., 230, 300-316; Fitzgerald, D. K., et al, Anal.Biochem., 36, 43-61; Gosselin, S., et al Anal.Biochem., 220, 92-97; Crawley, S. C., et al Anal.Biochem 185, 112-117; Yan, L., et al, Anal.Biochem., 223, 111-118; Yeh, J. C. and Cummings, R. D. (1996) Anal.Biochem., 236, 126-133; DeBose-Boyd, R. A., et al Arch.Biochem.Biophys., 335, 109-117; Rabina, J., et al, Anal.Biochem., 246, 71-78; Shedletzky, E., et al Anal.Biochem., 249, 88-93; Oubihi, M., et al Anal.Biochem., 257, 169-175; Kanie, Y., et al Anal.Biochem., 263, 240-245.)

A ligand or modulator of the invention may be converted using customary methods into pharmaceutical compositions. A ligand or modulator may be formulated into a pharmaceutical composition containing a ligand or modulator either alone or together with other active substances.

Ligands that are modulators that are capable of modulating the activity of glycosyltransferases have therapeutic and prophylactic potential. Therefore, the methods of the invention for identifying ligands or modulators may comprise one or more of the following additional steps:

(a) testing whether the ligand is a modulator of the activity of a glycosyltransferase, preferably testing the activity of the ligand in cellular assays and animal model assays;

(b) modifying the ligand;

(c) optionally rerunning steps (a) or (b); and

(d) preparing a pharmaceutical composition comprising the ligand.

Steps (a), (b) (c) and (d) may be carried out in any order, at different points in time, and they need not be sequential.

Still another aspect of the invention provides a method of conducting a drug discovery business comprising:

(a) providing one or more systems or methods for identifying modulators based on a model or structure of the present invention, preferably a method using a computer as described herein;

(b) conducting therapeutic profiling of modulators identified in step (a), or further analogs thereof, for efficacy and toxicity in animals; and

(c) formulating a pharmaceutical composition including one or more agents identified in step (b) as having an acceptable therapeutic profile.

In certain embodiments, the subject method may also include a step of establishing a distribution system for distributing the pharmaceutical composition for sale, and may optionally include establishing a sales group for marketing the pharmaceutical composition.

In yet another aspect of the invention, a method of conducting a target discovery business is provided comprising:

(a) providing one or more system or method for identifying modulators based on a model or structure of the present invention, preferably a method using a computer as described herein;

(b) optionally conducting therapeutic profiling of modulators identified in (a) for efficacy and toxicity in animals; and

(c) licensing to a third party the rights for further drug development and/or sales for agents identified in step (a), or analogs thereof.

There is also provided a pharmaceutical composition comprising a ligand or modulator, and a method of treating and/or preventing disease associated with a glycosyltransferase comprising the step of administering a ligand or modulator or pharmaceutical composition comprising a modulator to a patient.

In an aspect, the invention contemplates a method of treating a disease associated with a glycosyltransferase with inappropriate activity in a cellular organism, comprising:

(a) administering a ligand or modulator identified using the methods of the invention in an acceptable pharmaceutical preparation; and

(b) activating or inhibiting a glycosyltransferase to treat the disease.

The invention provides for the use of a ligand or modulator identified by the methods of the invention in the preparation of a medicament to treat or prevent a disease associated with or modulated by a glycosyltransferase in a cellular organism. Use of ligands or modulators of the invention to manufacture a medicament is also provided.

Another aspect of the invention provides machine readable media encoded with data representing a crystal or model of the invention or the coordinates of a structure of a glycosyltransferase or ligand binding pocket or binding site thereof as defined herein, or the three dimensional structure of a donor molecule or acceptor molecule or part thereof defined in relation to its spatial association with a three dimensional structure of a glycosyltransferase as defined herein. The invention also provides computerized representations of a crystal or model of the invention or the secondary, tertiary or quanternary structures of the invention, including any electronic, magnetic, or electromagnetic storage forms of the data needed to define the structures such that the data will be computer readable for purposes of display and/or manipulation. The invention further provides a computer programmed with a homology model of a ligand binding pocket of a glycosyltransferase. The invention still further contemplates the use of a homology model of the invention as input to a computer programmed for drug design and/or database searching and/or molecular graphic imaging in order to identify new ligands or modulators for glycosyltransferases.

These and other aspects of the present invention will become evident upon reference to the following detailed description and Tables, and attached drawings.

### DESCRIPTION OF THE DRAWINGS

The present invention will now be described only by way of example, in which reference will be made to the following Figures:

FIG. 1. Glycosyl transfer reactions. a) The LgtC catalyzed transfer of galactose from UDP-Gal to the LPS core oligosaccharide of Neisseria. The proposed mechanisms of b) an inverting and c) a retaining α-galactosyltransferase.

FIG. 2. An amino acid sequence alignment of Neisseria meningitidis LgtC and related enzymes from glycosyl transferase family 8. Secondary structure elements of LgtC are indicated above the sequence. Invariant residues are shown on blue background and conserved on orange. Sequences from the following organisms were used, their accession numbers in parenthesis. Neisseria meningitidis (P96945), Neisseria gonorrheae (Q50948), Pasturella multocida (AF237927), Haemophilus influenzae (P43947), Escherichia coli (Q92155), Salmonella tryphimurium (P19816), Helicobacter pylori (024967). Residues interacting with the UDP portion of the donor or with the galactose part are marked with filled circles and triangles respectively. Residues coordinating the metal are marked with stars and those that interact with 4-deoxylactose with diamonds.

FIG. 3. The overall architecture of LgtC. 3A. A C-α trace of the Lgtc monomer shown in stereo. 3B. The LgtC structure with bound substrate analogues. The substrates are depicted in CPK representation where the acceptor is coloured dark gray, the donor light gray and the manganese pink. Strands and helices are labelled. 3C. View of the LgtC structure showing the substrate binding N-terminal domain and the membrane attaching C-terminal domain. 3D. Topology diagram of LgtC. Helices are coloured blue and strands green.

FIG. 4. Stereo view of the active site. 4A. The ball-and stick models of the donor sugar UDP-Gal is colored as red sticks and the acceptor sugar lactose as green sticks in a refined 2fo-fc map contoured at 1.2 sigma. Amino acids interacting with the substrates are labeled. The loops that fold over the active site, residues 75-80 and 246-251, are colored in green. 4B. Molecular surface representation of the active site. UDP-Gal and 4-deoxylactose are shown in ball-and-stick form. UDP-Gal is almost completely buried in the enzyme while 4-deoxylactose is bound in an open pocket, more accessible to solvent 4C. The hydrogen bonding network of Q189 and the distance and angle to the anomeric carbon C1′.Distances are in Å.

FIG. 5. Schematic representation of the interactions between the enzyme and the substrate analogues. Hydrogen bonds (<3.1 Å for all bonds except Cys; <3.5 Å) are indicated with dashed lines. Vdw contacts are shown as nested half circles. Water molecules have not been included.

FIG. 6. Possible mechanisms in the LgtC catalyzed reaction. a) 6′-OH of the acceptor substrate and the b) side chain amide oxygen of Gln189 as potential nucleophiles. c) The mechanism of a hexosaminidase in which the amide oxygen of the substrate has been shown to function as the catalytic nucleophile.

### DESCRIPTION OF THE TABLES

The present invention will now be described only by way of example, in which reference will be made to the following Tables:

Table 1 shows a specific comparison of the specific activity and kinetic parameters of the mutants to the wild-type protein

Table 2 shows data collection, refinement statistics, and model steriochemistry.

Table 3 shows atomic interactions of a retaining glycosyltransferase and a donor molecule, and an acceptor molecule.

Table 4 shows the structural coordinates of a retaining galactosyltransferase LgtC from Neisseria meningitidis in complex with manganese and UDP 2-deoxy-2-fluoro-galactose.

Table 5 shows the structural coordinates of a retaining galactosyltransferase LgtC from Neisseria meningitidis in complex with manganese, UDP 2-deoxy-2-fluoro-galactose and 4-deoxylactose.

Table 6 shows the structural coordinates of a retaining galactosyltransferase LgtC from Neisseria meningitidis in complex with manganese and UDP 2-deoxy-2-fluoro-galactose and lactose.

In Tables 4 through 6 inclusive, from the left, the second column identifies the atom number; the third identifies the atom type; the fourth identifies the amino acid type; the fifth identifies the residue number; the sixth identifies the x coordinates; the seventh identifies the y coordinates; and the eighth identifies the z coordinates.

### DETAILED DESCRIPTION OF THE INVENTION

Unless otherwise indicated, all terms used herein have the same meaning as they would to one skilled in the art of the present invention. Practitioners are particularly directed to Current Protocols in Molecular Biology (Ansubel) for definitions and terms of the art.

Glycosyltransferase

The invention generally relates to glycosyltransferases and parts thereof. A glycosyltransferase enzyme is capable of transferring a particular sugar residue from a donor molecule to an acceptor molecule, thus forming a glycosidic linkage. Based on the type of donor sugar transferred, these enzymes are grouped into families, e.g. N-acetylglucosaminyltansferases, N-acetylgalactosaminyltransferases, mannosyltransferases, fucosyltransferases, galactosyltransferases, and sialyltransferases.

A retaining glycosyltransferases is one which transfers a sugar residue with the retention of anomeric configuration. An inverting glycosyltransferase, on the other hand, is one which transfers a sugar residue with the inversion of anomeric configuration. Campbell et al (1997) (as above) describes a classification of glycosyltransferases based on amino acid sequence similarities. Twenty-six families have been identified altogether, thirteen of which are designated as being inverting enzymes (Families 1, 2, 7, 9, 10, 11, 12, 13, 14, 16, 17, 18 and 23) and eight of which are designated as being retaining enzymes (Families 3, 4, 5, 6, 8, 15, 20 and 21).

In accordance with certain aspects of the invention, the enzyme is a retaining glycosyltransferase of Family 3, 4, 5, 6, 8, 15, 20 and 21 in the scheme of Campbell et al. In an aspect of the invention the enzyme is capable of catalyzing a step in the biosynthesis of a lipooligosaccharide or lipopolysaccharide. Preferably the glycosyltransferase is a member of Family 8 and has the activities of a lipopolysaccharide galactosyltransferase (EC 2.4.1.44), lipopolysaccharide glucosyltransferase 1 (EC 2.4.1.58), glycogenin glucosyltransferase (EC 2.4.1.186), inositol 1-α galactosyltransferase (EC 2.4.1.123). In a more preferred embodiment, the enzyme is a lipopolysaccharide galactosyltransferase [e.g. SwissProt P27128 (E. coli rfaI) and P19816 (S. typhimurium rfaI)] and in a most preferred embodiment the enzyme is a lipopolysaccharide galactosyltransferase of Neisseria (e.g. meningitidis, gonorrhoeae). A highly preferably enzyme is an α 1,4-galactosyltransferase from Neisseria menigitidis (GenBank Accession No. U65788).

Glycosyltransferases are derivable from a variety of sources, including viruses, bacteria, fungi, plants and animals. In a preferred embodiment the glycosyltransferase is derivable from a bacterium, in particular a gram-negative bacterium, such as one which is capable of acting as a pathogen. In an aspect of the invention the enzyme is derivable form a gram negative mucosal pathogen. For example, the glycosyltanferase may be found in one (or more) of the following organisms: Neisseria, Escherichia, Salmonella, Haemophilus, Moraxella, Bordatella, and Campylobacter. In a preferred embodiment the enzyme is found in a bacteria of the genus Neisseria, for example N. meningitidis or N. gonorrhea. In a highly preferred embodiment, the enzyme is found in N. meningitidis.

Preferably the glycosyltransferase is derivable from an organism possessing a lipooligosaccharide (LOS). The lipooligosaccharide may mimic human glycolipids in order to avoid detection by the immune system. For example, the LOS may mimic human lacto-N-neotetraose, sialylacto-N-neotetraose and/or the P^{k} blood group glycolipid. In a highly preferred embodiment, the enzyme is capable of adding an α-galactose to a terminal lactose of the LOS structure, creating a P^{k} blood group glycolipid mimic.

A glycosyltransferase or part thereof in the present invention may be a wild type enzyme, or part thereof, or a mutant, variant or homologue of such an enzyme.

The term “wild type” refers to a polypeptide having a primary amino acid sequence which is identical with the native enzyme (for example, the bacterial enzyme).

The term “mutant” refers to a polypeptide having a primary amino acid sequence which differs from the wild type sequence by one or more amino acid additions, substitutions or deletions. Preferably, the mutant has at least 90% sequence identity with the wild type sequence. Preferably, the mutant has 20 mutations or less over the whole wild-type sequence. More preferably the mutant has 10 mutations or less, most preferably 5 mutations or less over the whole wild-type sequence. A mutant may or may not be functional.

The term “variant” refers to a naturally occurring polypeptide which differs from a wild-type sequence. A variant may be found within the same species (i.e. if there is more than one isoform of the enzyme) or may be found within a different species. Preferably the variant has at least 90% sequence identity with the wild type sequence. Preferably, the variant has 20 mutations or less over the whole wildtype sequence. More preferably, the variant has 10 mutations or less, most preferably 5 mutations or less over the whole wild-type sequence.

The term “part” indicates that the polypeptide comprises a fraction of the wild-type amino acid sequence. It may comprise one or more large contiguous sections of sequence or a plurality of small sections. The “part” may comprise a ligand binding pocket as described herein. The polypeptide may also comprise other elements of sequence, for example, it may be a fusion protein with another protein (such as one which aids isolation or crystallisation of the polypeptide). Preferably the polypeptide comprises at least 50%, more preferably at least 65%, most preferably at least 80% of the wild-type sequence.

The term “homologue” means a polypeptide having a degree of homology with the wild-type amino acid sequence. The term “homology” can be equated with “identity”.

In the present context, a homologous sequence is taken to include an amino acid sequence which may be at least 75, 85 or 90% identical, preferably at least 95 or 98% identical to the wild-type sequence. Typically, the homologues will comprise the same sites (for example LBP) as the subject amino acid sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences (e.g. Wilbur, W. J. and Lipman, D. J. Proc. Natl. Acad. Sci. USA (1983), 80:726-730).

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nln.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may have deletions, insertions or substitutions of amino acid residues which produce a silent change and result in a functionally equivalent enzyme. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The polypeptide may also have a homologous substitution (substitution and replacement are both used herein to mean the interchange of an existing amino acid residue, with an alternative residue) i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-a-amino butyric acid*, L-γ-amino butyric acid*, L-α-amino isobutyric acid*, L-ε-amino caproic acid#, 7-amino heptanoic acid*, L-methionine sulfone#*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino), L-Tyr (methyl)*, L-Phe (4-isopropyl)*, L-Tic (1,2,3,4-tetrahydroisoquinoline-3-carboxyl acid)*, L-diaminopropionic acid# and L-Phe (4-benzyl)*. The notation * has been utilised for the purpose of the discussion above (r lating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involving the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

Crystal

The invention provides a crystal of a retaining glycosyltransferase or a part or fragment thereof, in particular a ligand binding pocket of a glycosyltransferase.

As used herein, the term “crystal” or “crystalline” means a structure (such as a three dimensional (3D) solid aggregate) in which the plane faces intersect at definite angles and in which there is a regular structure (such as internal structure) of the constituent chemical species. Thus, the term “crystal” can include any one of: a solid physical crystal form such as an experimentally prepared crystal, a crystal structure derivable from the crystal (including secondary and/or tertiary and/or quaternary structural elements), a 2D and/or 3D model based on the crystal structure, a representation thereof such as a schematic representation thereof or a diagrammatic representation thereof, or a data set thereof for a computer. In one aspect, the crystal is usable in X-ray crystallography techniques. Here, the crystals used can withstand exposure to X-ray beams used to produce a diffraction pattern data necessary to solve the X-ray crystallographic structure. A crystalline form of a glycosyltransferase, may be characterized as being capable of diffracting x-rays in a pattern defined by one of the crystal forms depicted in Blundel et al 1976, Protein Crystallography, Academic Press.

A crystal of the invention comprises an N-terminal α/β pocket with a central core β-sheet characterized by the following: seven strands (β3, β2, β1, β4, β7, β6, β8) all of which are parallel with the exception of β7; the first 100 residues form a nucleotide binding fold composed of four parallel strands sandwiched between two helices (i.e. A and B) on one side and two helices (i.e. C and D) on the other; and the remainder of the core β-sheet is flanked by three α-helices on one side and five on the other.

The crystal may also be characterized by an antiparallel β-ribbon formed by β5 and β9 lying almost perpendicular to the core β3-sheet, and a substrate binding cleft that lies along the base of the core β3-sheet.

The C-terminal domain that mediates membrane attachment of a bacterial glycosyltransferase may be predominantly helical. In particular, it may comprise two helices [i.e. helix M and helix N (which is 3₁₀ in nature)] that form a small pedestal that packs perpendicular to the helices of the nucleotide binding motif and to the β-ribbon.

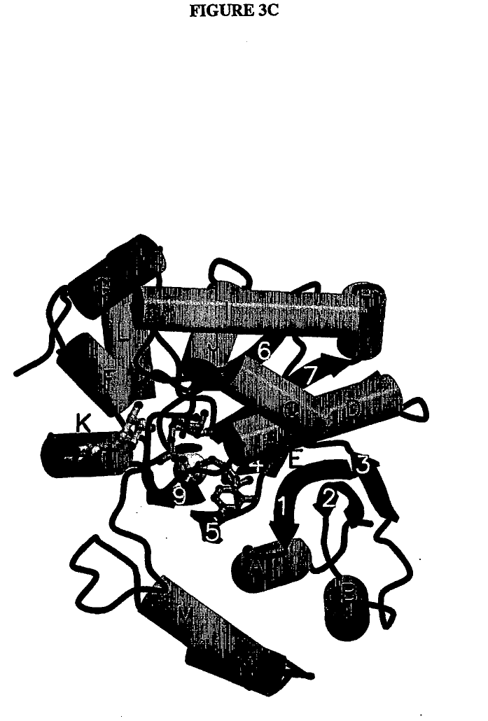

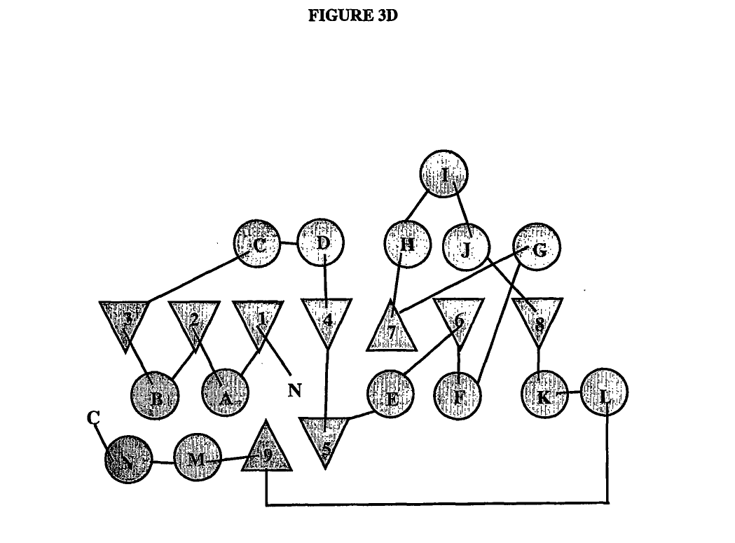

An illustration of a structure of the invention is shown in FIG. 3.

In an embodiment, a crystal of a glycosyltransferase of the invention belongs to space group P2₁2₁2₁. The term “space group” refers to the lattice and symmetry of the crystal. In a space group designation the capital letter indicates the lattice type and the other symbols represent symmetry operations that can be carried out on the contents of the asymmetric unit without changing its appearance

A crystal of the invention may comprise a unit cell having the following unit dimensions: a=37.79 (∀0.05) Å, b=76.05 (∀0.05) Å, c=86.84 (∀0.05) Å. The term “unit cell” refers to the smallest and simplest volume element (i.e. parallelpiped-shaped block) of a crystal that is completely representative of the unit of pattern of the crystal. The unit cell axial lengths are represented by a, b, and c. Those of skill in the art understand that a set of atomic coordinates determined by X-ray crystallography is not without standard error.

In a preferred embodiment, a crystal of the invention has the structural coordinates of the enzyme as shown in Table 4, Table 5, or Table 6. As used herein, the term “structural coordinates” refers to a set of values that define the position of one or more amino acid residues with reference to a system of axes. A data set of structural coordinates defines the three dimensional structure of a molecule or molecules. The term refers to a data set that defines the three dimensional structure of a molecule or molecules (e.g. Cartesian coordinates, temperature factors, and occupancies). Structural co-ordinates can be slightly modified and still render nearly identical three dimensional structures. A measure of a unique set of structural coordinates is the root-mean-square deviation of the resulting structure. Structural coordinates that render three dimensional structures (in particular a three dimensional structure of a ligand binding pocket) that deviate, from one another by a root-mean-square deviation of less than 5 Å, 4 Å, 3 Å, 2 Å, or 1.5 Å may be viewed by a person of ordinary skill in the art as very similar.

Variations in structural coordinates may be generated because of mathematical manipulations of the. structural coordinates of a glycosyltransferase described herein. For example, the structural coordinates of Table 4, 5, or 6 may be manipulated by crystallographic permutations of the structural coordinates, fractionalization of the structural coordinates, integer additions or substractions to sets of the structural coordinates, inversion of the structural co-ordinates or any combination of the above.

Variations in the crystal structure due to mutations, additions, substitutions, and/or deletions of the amino acids, or other changes in any of the components that make up the crystal may also account for modifications in structural coordinates. If such modifications are within an acceptable standard error as compared to the original structural coordinates, the resulting structure may be the same. Therefore, a ligand that bound to a ligand binding pocket of an α1,4-galactosyltransferase would also be expected to bind to another ligand binding pocket whose structural coordinates defined a shape that fell within the acceptable error. Such modified structures of a ligand binding pocket thereof are also within the scope of the invention.

Various computational analyses may be used to determine whether a ligand or a ligand binding pocket thereof is sufficiently similar to all or parts of a ligand or a ligand binding pocket thereof. Such analyses may be carried out using conventional software applications and methods as described herein.

A crystal of the invention may also be specifically characterised by the parameters, diffraction statistics and/or refinement statistics set out in Table 2.

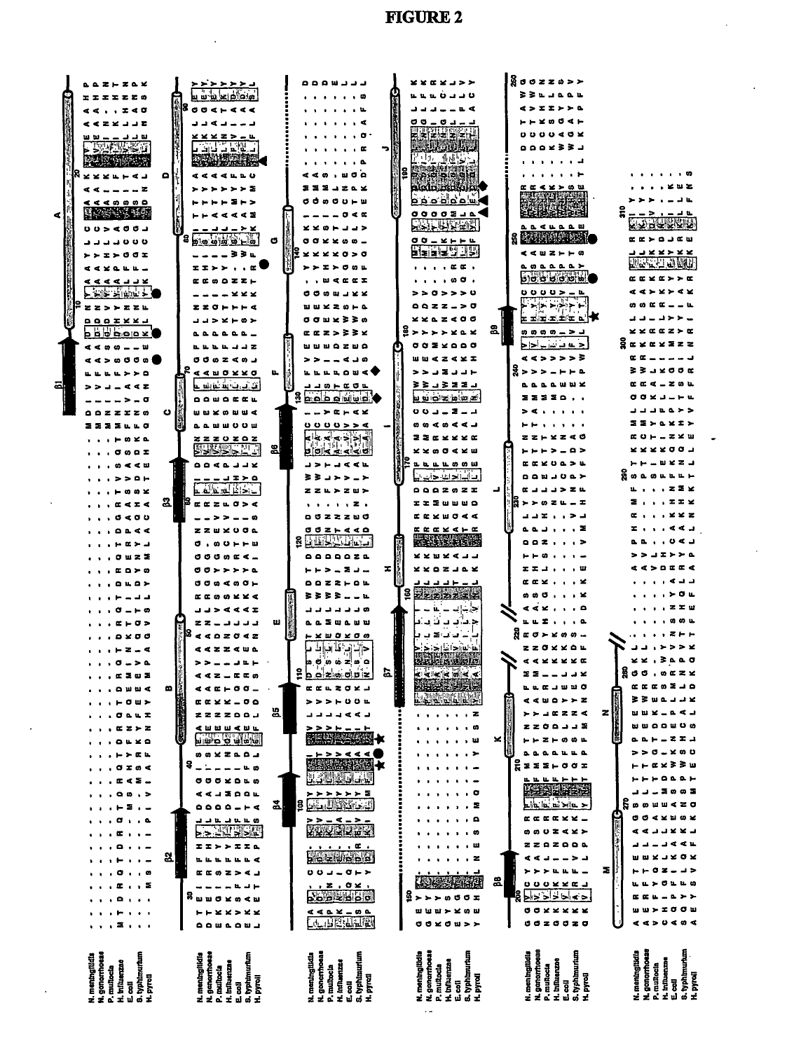

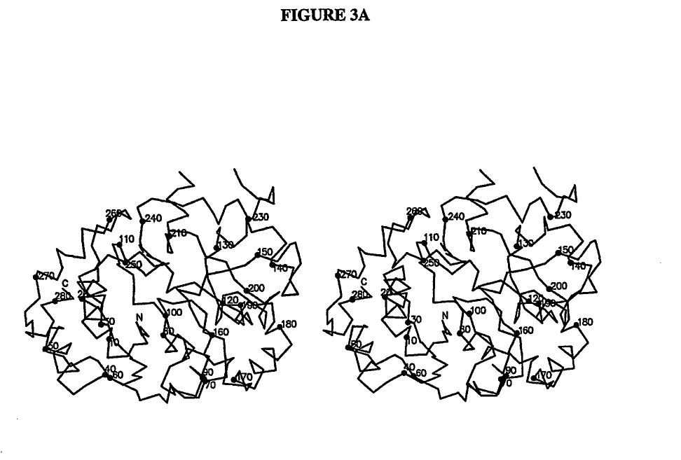

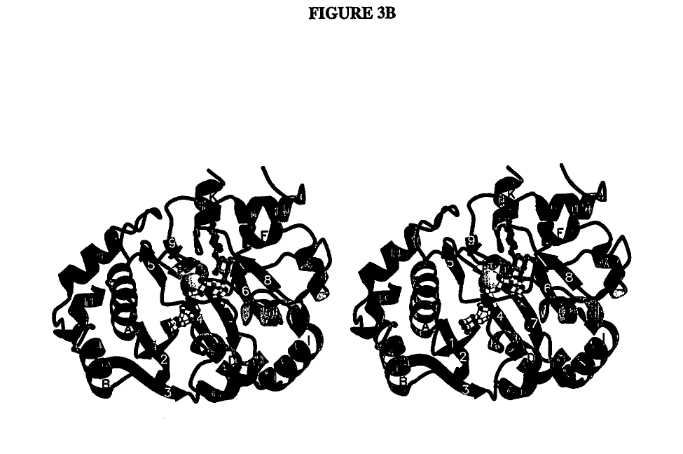

A crystal of the invention may comprise the entire sequence of a retaining galactosyltransferase, preferably from glycosyltransferase family 8 (e.g. see FIG. 2), preferably an α1,4-galactosyltransferase, and most preferably an α-1,4-galactosyltransferase (LgtC) derivable from Neisseria meningitidis. A crystal of the invention may comprise a sequence of a retaining galactosyltransferase with a deletion in or around the C-terminus. Preferably the deletion and/or mutation in the C-terminus is sufficient to facilitate crystallisation of the protein. A crystallized enzyme may not include the portion of a bacterial glycosyltransferase enzyme that attaches to the surface of a bacterial membrane. For example, the C-terminal 25 to 50 amino acid residues may be deleted from an α-1,4-galactosyltransferase (LgtC) derivable from Neisseria meningitidis.

Ligand-Binding Pocket

In an embodiment the invention provides a crystal comprising a ligand binding pocket.

“Ligand binding pocket” or “LBP” refers to a region of a molecule or molecular complex that as a result of its shape, favourably associates with a ligand or a part thereof. For example, it may be a region of a glycosyltransferase that is responsible for binding a ligand including a donor molecule, an acceptor molecule and/or a sugar during transfer (e.g. active site binding pocket). With reference to the models and structures of the invention, residues in a ligand binding pocket may be defined by their spatial proximity to a ligand in a model or structure.

A “ligand” refers to a compound or entity that associates with a ligand binding pocket, including substrates such as acceptor molecules or analogues or parts thereof, donor molecules or analogues or parts thereof A ligand may be designed rationally by using a model according to the present invention. A ligand may be a modulator of a glycosyltransferase including an inhibitor.

A “donor molecule” or “sugar nucleotide donor” refers to a molecule capable of donating a sugar to an acceptor molecule, via the action of a glycosyltransferase enzyme. The donor molecule may be di- or poly-saccharides, sugar 1-phosphates, or, most commonly, nucleotide diphosphosugars (NDP-sugars), or nucleotide phosphosugars. In a preferred embodiment, the donor molecule is UDP-galactose, UDP glucose, UDP mannose, UDP-N-acetylglucosamine, UDP-N-acetylgalactosamine, UDP-N-acetymannosamine, UDP-glucuronic acid, UDP-galacturonic acid, UDP-fucose, UDP-xylose, UDP-rhamnose, and ADP, GDP and TDP derivatives thereof, and CMP sialic acid.

An acceptor molecule is capable of accepting a sugar from a donor molecule, via the action of a glycosyltransferase enzyme. It may, for example, comprise a terminal sugar residue for transfer purposes. The acceptor molecule or aglycone can be, for example, a lipid, a protein, a heterocyclic compound, an antibiotic, a peptide, an amino acid, an aromatic or aliphatic alcohol or thiol or another carbohydrate residue. In a preferred embodiment, the donor molecule is or comprises a terminal lactose.

An analogue of a donor or acceptor molecule is one which mimics the donor or acceptor molecule, binding in the LBP, but which is incapable (or has a significantly reduced capacity) to take part in the transfer reaction. For example, UDP-Gal can act as a donor sugar for a galactosyltransferase. UDP-2Fgal, on the other hand, acts as a donor sugar analogue. The fluorine at the 2-position destabilises the transition state for the transfer reaction, so that effectively no transfer occurs. Similarly, UDPGlcNAc can act as a donor sugar for GnT1 and the methylene phosphonate analogue can act as a donor sugar analogue. By the same token, a terminal lactose can act as an acceptor sugar for a α1,4 galactosyltransferase. 4-deoxylactose is non reactive and is an example of an acceptor molecule analogue.

The term “ligand binding pocket” (LBP) includes a homologue of the ligand binding pocket or a portion thereof. As used herein, the term “homologue” in reference to a ligand binding pocket refers to ligand binding pocket or a portion thereof which may have deletions, insertions or substitutions of amino acid residues as long as the binding specificity of the molecule is retained. In this regard, deliberate amino acid substitutions may be made on the basis of similarity in polarity, charge, solubility, hydrophobicity, hydrophilicity, and/or the amphipathic nature of the residues as long as the binding specificity of the ligand binding pocket is retained.

As used herein, the term “portion thereof” means the structural co-ordinates corresponding to a sufficient number of amino acid residues of the glycosyltransferase LBP (or homologues thereof) that are capable of associating or interacting with a ligand. This term includes glycosyltransferase ligand binding pocket amino acid residues having amino acid residues from about 4 Å to about 5 Å of an associated ligand or part thereof. Thus, for example, the structural co-ordinates provided in a crystal structure may contain a subset of the amino acid residues in the LBP which may be useful in the modelling and design of compounds that bind to the LBP.

A crystal of the invention may comprise a ligand binding pocket and at least part of the pocket which may be involved in attaching the enzyme to the bacterial membrane. Preferably the crystal comprises the entire sequence of the enzyme, optionally with a deletion in or around the C-terminus. Preferably the deletion and/or mutation in the C-terminus is sufficient to facilitate crystallisation of the protein.

A ligand-binding pocket may comprise an active site binding pocket of a glycosyltransferase. The active site binding pocket refers to the region of a glycosyltransferase where the transfer of a sugar from the donor molecule to the acceptor occurs. In accordance with one aspect, the invention contemplates a crystal of an active site binding pocket of a retaining glycosyltransferase comprising a core β-sheet containing 7 strands (β3, β2, β1, β4, β6, β8 in FIG. 3) all of which are parallel with the exception of β7; the core β-sheet further characterized by a nucleotide binding motif composed of four parallel strands sandwiched between helices A and B on one side and helices C and D on the other as shown in FIG. 3. A polypeptide comprising an active site binding pocket with the shape and structure of an active site binding pocket described herein is also within the scope of the invention.

The ligand binding pocket may comprise a pocket of a glycosyltransferase structure described herein that is capable of associating with a donor molecule, preferably a nucleotide or portion thereof. A ligand binding pocket may comprise the amino acid residues at the C-terminus of β1 and the N-terminus of helix A of a glycosyltransferase structure as described herein, that are capable of associating with a nucleotide of a donor molecule as described herein. In particular, a ligand-binding pocket may comprise one or both of the loops that associate with a donor molecule or analogue. Such a ligand binding pocket may comprise a loop comprising residues 75-80 and/or a loop comprising residues 246-251 of a glycosyltransferase described herein or a homologue thereof.

A ligand binding pocket may comprise a cleft at the C-terminal end of a glycosyltransferase β-sheet structure as described herein that is capable of associating with a uridine diphosphate. The ligand binding pocket may comprise a conserved Tyr 11 (Phe in E. coli and Salmonella) of a glycosyltransferase structure as described herein that is capable of stacking with a uracil base.

When UDP-Gal is capable of acting as a donor molecule for the glycosyltransferase enzyme, preferably the ligand binding pocket may comprise at least one of the residues involved in binding to the UDP portion of UDP-Gal, namely: Tyr 11, Asn 10, Asp 8, Ala 6, Ile 104, Lys 250, Gly 247 and His 78 or a homologue thereof.

When UDP-Gal is capable of acting as a donor molecule for the glycosyltransferase enzyme, preferably the ligand binding pocket may comprise at least one of the residues involved in binding to the UDP portion of UDP-Gal, namely: Asp 8, Asn 10, Ala 6, Ile 104, Lys 250, Gly 247, and His 78 or a homologue thereof.

Alternatively, or more preferably in addition, the ligand binding pocket may comprise at least one of the residues involved in shielding the reactive center C1′ atom from water, namely: Ile 76, Asp 103, Asp 153, Ala 154, Gly 155, Tyr 186, Gln 189, His 244, Cys 246 and Gly 247; or a homologue thereof.

Based on the crystal of LgtC herein, Gln 189 may act as the nucleophile during the transfer reaction. Hence, in an embodiment a crystal of the invention comprises Gln 189 or a homologue thereof. Preferably, the Glnl89 is oriented through hydrogen bonds to both sugar (donation of a hydrogen bond from Nεl to O6 of lactose) and conserved protein side chains (acceptance of a hydrogen bond from the side chain Nε2 of Asn153).

Alternatively, or more preferably in addition, the ligand binding pocket may comprise at least one of the residues involved in binding to a sugar moiety of a donor molecule such as the galactosyl moiety of UDP-Gal, namely: Asp103, Arg 86, Asp 188, and optionally one or more of Asn 153, Val 79, Thr 83, Gln 187 and Gln 189 or a homologue thereof.

When lactose is capable of acting as an acceptor molecule for the glycosyltransferase. enzyme, the ligand binding pocket may comprise at least one of the residues involved in binding to lactose, namely: Asp130, Gln 189, Val 76, His 78, Tyr 186, Cys 246, Gly 247, Phe 132, Pro 211, Pro 248, Thr 212 and Cys 246; or a homologue thereof.

With reference to a crystal of the present invention, residues in the LBP may be defined by their spatial proximity to a ligand in the crystal structure. For example, such may be defined by their proximity to a donor and/or an acceptor molecule.

A ligand binding pocket may comprise one or more of the residues involved in co-ordination of a Mn²⁺ ion, namely: His 244, Asp 103 and Asp 105; or a homologue thereof.

Preferably a LBP comprises at least one DXD motif. A “DXD” sequence motif is common to a wide range of glycosyltransferases, both in prokaryotes and eukaryotes, even though they may not share other sequence similarities. (Campbell et al., 1997 Biochemical Journal 326, 929-939, Breton et al., 1998 Journal of Biochemistry 123, 1000-1009 and Kapitonov and Yu, 1999 Glycobiology 9, 961-978). This motif has been proposed to be involved in the coordination of a divalent cation in the binding of the nucleotide sugar (Busch et al., 1998 The Journal of Biological Chemistry 273, 19566-19572). A number of mutagenesis studies have been carried out in various species on the conserved aspartate residues in the DXD sequence and all have found that enzymatic activity is completely abolished upon removal of the carboxylate (Shibayama et al., 1998 Journal of Bacteriology 180, 5313-5318, Wiggins and Munro, 1998 Proc. Natl. Acad Sci. USA 95, 7945-7950, Busch et al., 1998 (as above) and Hagen et al., 1999 The Journal of Biological Chemistry 274, 6797-6803).

A ligand binding pocket may comprise one or more of the amino acid residues for a glycosyltransferase structure of the invention identified by atomic contacts on the enzyme for atomic interactions numbers 1 through 17 shown in Table 3.

In an embodiment of the invention, a crystal or secondary, tertiary, and/or quanternary structure of a ligand binding pocket of a glycosyltransferase that associates with a diphosphate of a sugar nucleotide donor molecule is provided comprising one or both of the enzyme atomic contacts of atomic interactions 6 and 7 identified in Table 3, each atomic interaction defined therein by an atomic contact (more preferably, a specific atom where indicated) on the diphosphate group, and an atomic contact (more preferably, a specific amino acid residue where indicated) on the glycosyltransferase (i.e. enzyme atomic contact). Preferably, a crystal or secondary, tertiary, and/or quanternary structure of a ligand binding pocket is defined by the atoms of the enzyme atomic contacts of atomic interactions 6 and/or 7 having the structural coordinates for the atoms listed in Table 4, 5, or 6.

In an embodiment of the invention, a crystal or secondary, tertiary, and/or quanternary structure of a ligand binding pocket of a glycosyltransferase that associates with a heterocyclic amine base (preferably uracil) of a sugar nucleotide donor molecule is provided comprising one, two, or three of the enzyme atomic contacts of atomic interactions 1,2, and 3 identified in Table 3, each atomic interaction defined therein by an atomic contact (more preferably, a specific atom where indicated) on the heterocyclic amine base, and an atomic contact (more preferably, a specific amino acid residue where indicated) on the glycosyltransferase (i.e. enzyme atomic contact). Preferably, a crystal or secondary, tertiary, and/or quanternary structure of a ligand binding pocket is defined by the atoms of the enzyme atomic contacts of atomic interactions 1, 2, and/or 3 having the structural coordinates for the atoms listed in Table 4, 5, or 6.

In an embodiment of the invention, a crystal or secondary, tertiary, and/or quanternary structure of a ligand binding pocket of a glycosyltransferase that associates with a sugar of the nucleotide (preferably ribose) of a sugar nucleotide donor molecule is provided comprising one or both of the enzyme atomic contacts of atomic interactions 4 and 5 identified in Table 3, each atomic interaction defined therein by an atomic contact (more preferably, a specific atom where indicated) on the sugar, and an atomic contact (more preferably, a specific amino acid residue where indicated) on the glycosyltransferase (i.e. enzyme atomic contact). Preferably, a crystal or secondary, tertiary, and/or quanternary structure of a ligand binding pocket is defined by the atoms of the enzyme atomic contacts of atomic interactions 4 and/or 5 having the structural coordinates for the atoms listed in Table 4, 5, or 6.

In an embodiment of the invention, a crystal or secondary, tertiary, and/or quanternary structure of a ligand binding pocket of a glycosyltransferase that associates with a sugar to be transferred (e.g. Gal) of a sugar nucleotide donor molecule is provided comprising one, two, or three enzyme atomic contacts of atomic interactions 8, 9, and 10 identified in Table 3, each atomic interaction defined therein by an atomic contact (more preferably, a specific atom where indicated) on the selected sugar, and an atomic contact (more preferably, a specific amino acid residue where indicated) on the glycosyltransferase (i.e. enzyme atomic contact). Preferably, a crystal or secondary, tertiary, and/or quanternary structure of a ligand binding pocket is defined by the atoms of the enzyme atomic contacts of atomic interactions 8, 9, and/or 10 having the structural coordinates for the atoms listed in Table 4, 5, or 6.

In an embodiment of the invention, a crystal or secondary, tertiary, and/or quanternary structure of a ligand binding pocket of a glycosyltransferase that associates with a nucleotide (preferably UDP) of a sugar nucleotide donor molecule is provided comprising one, two, three, four, five, six, or seven enzyme atomic contacts of atomic interactions 1 through 7 identified in Table 3, each atomic interaction defined therein by an atomic contact (more preferably, a specific atom where indicated) on the nucleotide, and an atomic contact (more preferably, a specific amino acid residue where indicated) on the glycosyltransferase (i.e. enzyme atomic contact). Preferably, a crystal or secondary, tertiary, and/or quanternary structure of a binding pocket is defined by the atoms of one, two, three, four, five, six, or seven enzyme atomic contacts of atomic interactions 1 through 7 having the structural coordinates for the atoms listed in Table 4, 5, or 6.

In an embodiment of the invention, a crystal or secondary, tertiary, and/or quanternary structure of a ligand binding pocket of a glycosyltransferase that associates with a sugar nucleotide donor molecule (e.g. UDP-Gal) is provided comprising one, two, three, four, five, six, seven, eight, nine, or ten enzyme atomic contacts of atomic interactions 1 through 10 identified in Table 3, each atomic interaction defined therein by an atomic contact (more preferably, a specific atom where indicated) on the sugar nucleotide donor molecule, and an atomic contact (more preferably, a specific amino acid residue where indicated) on the glycosyltransferase (i.e. enzyme atomic contact). Preferably a crystal or secondary, tertiary, and/or quanternary structure of a ligand binding pocket is defined by the atoms of one, two, three, four, five, six, seven, eight, nine, or ten enzyme atomic contacts of atomic interactions 1 through 10 having the structural coordinates for the atoms listed in Table 4, 5, or 6.

In an embodiment of the invention, a crystal or secondary, tertiary, and/or quanternary structure of a ligand binding pocket of a glycosyltransferase that associates with an acceptor molecule (e.g. acceptor with a terminal lactose) is provided comprising one two, three, four, five, or six enzyme atomic contacts of atomic interactions 12 through 17 identified in Table 3, each atomic interaction defined therein by an atomic contact (more preferably, a specific atom where indicated) on the acceptor molecule, and an atomic contact (more preferably, a specific amino acid residue where indicated) on the glycosyltransferase (i.e. enzyme atomic contact). Preferably a crystal or secondary, tertiary, and/or quanternary structure of a ligand binding pocket is defined by the atoms of one, two, three, four, five, or six enzyme atomic contacts of atomic interactions 12 through 17 having the structural coordinates for the atoms listed in Table 4, 5, or 6.

Complexes

A crystal of the invention includes a crystalline glycosyltransferase or part thereof (e.g. ligand binding pocket) in association with one or more moieties, including heavy-metal atoms i.e. a derivative crystal, a metal cofactor, or one or more ligands or molecules i.e. a co-crystal.

The term “associate”, “association” or “associating” refers to a condition of proximity between a moiety (i.e. chemical entity or compound or portions or fragments thereof), and a glycosyltransferase, or parts or fragments thereof (e.g. ligand binding pockets). The association may be non-covalent i.e. where the juxtaposition is energetically favoured by for example, hydrogen-bonding, van der Waals, or electrostatic or hydrophobic interactions, or it may be covalent.

The term “heavy-metal atoms” refers to an atom that can be used to solve an x-ray crystallography phase problem, including but not limited to a transition element, a lanthanide metal, or an actinide metal. Lanthanide metals include elements with atomic numbers between 57 and 71, inclusive. Actinide metals include elements with atomic numbers between 89 and 103, inclusive.

Multiwavelength anomalous diffraction (MAD) phasing may be used to solve protein structures using selenomethionyl (SeMet) proteins. Therefore, a complex of the invention may comprise a crystalline glycosyltransferase or part thereof (e.g. ligand binding pocket) with selenium associated with the methionine residues of the protein.

In an embodiment of the invention, a ligand binding pocket is in association with a metal cofactor in the crystal. A “metal cofactor” refers to a metal required for glycosyltransferase activity and/or stability. For example, the metal cofactor may be manganese, and other similar atoms or metals. Different glycosyltransferases may require different cofactors, for example Mn²⁺, Mg²⁺, Co²⁺, Zn²⁺, Fe²⁺, and Ca²⁺. In a preferred embodiment the LBP is in association with manganese.

A ligand binding pocket in a complex with a cofactor preferably comprises one or more of the residues involved in co-ordination of a Mn2+ ion, namely: His 244, Asp 103 and Asp 105; or a homologue thereof. Preferably the LBP comprises at least one DXD motif.

A crystal may comprise a complex between a ligand-binding pocket and one or more ligands or molecules. In other words the ligand binding pocket may be associated with one or more ligands or molecules in the crystal. The ligand may be any compound which is capable of stably and specifically associating with the ligand binding pocket A ligand may, for example, be a substrate such as a donor or an acceptor molecule or analogue thereof, and/or the ligand may be a modulator of the glycosyltransferase.

Therefore, the present invention also provides:

(a) a crystal comprising a ligand binding pocket of a glycosyltransferase and a donor molecule or analogue thereof;

(b) a crystal comprising a ligand binding pocket of a glycosyltransferase and an acceptor molecule or analogue thereof;

(c) a crystal comprising a ligand binding pocket of a glycosyltransferase and a donor molecule or analogue thereof, and an acceptor molecule thereof .

A complex may comprise one or more of the atomic interactions identified in Table 3. A structure of a complex of the invention may be defined by selected atomic interactions, preferably the atomic interactions as defined in Table 3.

In an embodiment of the invention, a crystal or secondary, tertiary, and/or quanternary structure of a ligand binding pocket of a glycosyltransferase in association with a diphosphate of a sugar nucleotide donor molecule is provided comprising one or both of atomic interactions 6 and 7 identified in Table 3, each atomic interaction defined therein by an atomic contact (more preferably, a specific atom where indicated) on the diphosphate group, and an atomic contact (more preferably, a specific amino acid residue where indicated) on the glycosyltransferase (i.e. enzyme atomic contact). Preferably, a crystal or secondary, tertiary, and/or quanternary structure of such a complex is defined by the atoms of the atomic contacts of the atomic interactions having the structural coordinates for the atoms listed in Table 4, 5, or 6.

In an embodiment of the invention, a crystal or secondary, tertiary, and/or quanternary structure of a ligand binding pocket of a glycosyltransferase in association with a heterocyclic amine base (preferably uracil) of a sugar nucleotide donor molecule is provided comprising one, two, or three of atomic interactions 1, 2, and 3 identified in Table 3, each atomic interaction defined therein by an atomic contact (more preferably, a specific atom where indicated) on the heterocyclic amine base, and an atomic contact (more preferably, a specific amino acid residue where indicated) on the glycosyltransferase (i.e. enzyme atomic contact). Preferably, a crystal or secondary, tertiary, and/or quanternary structure of a such a complex is defined by the atoms of the atomic contacts of the atomic interactions having the structural coordinates for the atoms listed in Table 4, 5, or 6.

In an embodiment of the invention, a crystal or secondary, tertiary, and/or quanternary structure of a ligand binding pocket of a glycosyltransferase in association with a sugar of the nucleotide (preferably ribose) of a sugar nucleotide donor molecule is provided comprising one or both of atomic interactions 4 and. 5 identified in Table 3, each atomic interaction defined therein by an atomic contact (more preferably, a specific atom where indicated) on the sugar, and an atomic contact (more preferably, a specific amino acid residue where indicated) on the glycosyltransferase (i.e. enzyme atomic contact). Preferably, a crystal or secondary, tertiary, and/or quanternary structure of such a complex is defined by the atoms of the atomic contacts of the atomic interactions having the structural coordinates for the atoms listed in Table 4, 5, or 6.

In an embodiment of the invention, a crystal or secondary, tertiary, and/or quanternary structure of a ligand binding pocket of a glycosyltransferase in association with a sugar to be transferred (e.g. Gal) of a sugar nucleotide donor molecule is provided comprising one, two, or three of atomic interactions 8, 9, and 10 identified in Table 3, each atomic interaction defined therein by an atomic contact (more preferably, a specific atom where indicated) on the selected sugar, and an atomic contact (more preferably, a specific amino acid residue where indicated) on the glycosyltransferase (i.e. enzyme atomic contact). Preferably, a crystal or secondary, tertiary, and/or quanternary structure of such a complex is defined by the atoms of the atomic contacts of the atomic interactions having the structural coordinates for the atoms listed in Table 4, 5, or 6.

In an embodiment of the invention, a crystal of a ligand binding pocket or secondary, tertiary, and/or quanternary structure of a glycosyltransferase in association with a nucleotide (preferably UDP) of a sugar nucleotide donor molecule is provided comprising one, two, three, four, five, six, or seven of atomic interactions 1 through 7 identified in Table 3, each atomic interaction defined therein by an atomic contact (more preferably, a specific atom where indicated) on the nucleotide, and an atomic contact (more preferably, a specific amino acid residue where indicated) on the glycosyltransferase (i.e. enzyme atomic contact). Preferably, a crystal or secondary, tertiary, and/or quanternary structure of such a complex is defined by the atoms of the atomic contacts of the atomic interactions having the structural coordinates for the atoms listed in Table 4, 5, or 6.

In an embodiment of the invention, a crystal or secondary, tertiary, and/or quanternary structure of a ligand binding pocket of a glycosyltransferase in association with a sugar nucleotide donor molecule (e.g. UDP-Gal) is provided comprising one, two, three, four, five, six, seven, eight, nine, or ten of atomic interactions 1 through 10 identified in Table 3, each atomic interaction defined therein by an atomic contact (more preferably, a specific atom where indicated) on the sugar nucleotide donor molecule, and an atomic contact (more preferably, a specific amino acid residue where indicated) on the glycosyltransferase (i.e. enzyme atomic contact). Preferably a crystal or secondary, tertiary, and/or quanternary structure of such a complex is defined by the atoms of the atomic contacts of atomic interactions having the structural coordinates for the atoms listed in Table 4, 5, or 6.

In an embodiment of the invention, a crystal or secondary, tertiary, and/or quanternary structure of a ligand binding pocket of a glycosyltransferase in association with an acceptor molecule (e.g. acceptor with a terminal lactose) is provided comprising one two, three, four, five, or six of atomic interactions 12 through 17 identified in Table 3, each atomic interaction defined therein by an atomic contact (more preferably, a specific atom where indicated) on the acceptor molecule, and an atomic contact (more preferably, a specific. amino acid residue where indicated) on the glycosyltransferase (i.e. enzyme atomic contact). Preferably a crystal or secondary, tertiary, and/or quanternary structure of such a complex is defined by the atoms of the atomic contacts of the atomic interactions having the structural coordinates for the atoms listed in Table 4, 5, or 6.

In an embodiment a crystal of the invention comprises a ligand binding pocket of a galactosyltransferase in association with a donor molecule, such as UDP-2Fgal and/or an acceptor molecule such as 4-deoxylactose or lactose. These complexes may have the structural coordinates shown in Table 4, 5, or 6.

A crystal of the invention may enable the determination of structural data for the donor molecule or acceptor molecule. In order to be able to derive structural data for the donor/acceptor molecule, it is necessary for the molecule to have sufficiently strong electron density to enable a model of the molecule to be built using standard techniques. For example, there should be sufficient electron density to allow a model to be built using XTALVIEW (McRee 1992 J. Mol. Graphics. 10 44-46).

Method of Making a Crystal

The present invention also provides a method of making a crystal according to the invention. The crystal may be formed from an aqueous solution comprising a purified polypeptide comprising a glycosyltransferase or part or fragment thereof (e.g. a catalytic portion, ligand binding pocket). A method may utilize a purified polypeptide comprising a glycosyltransferase ligand binding pocket to form a crystal

The term “purified” in reference to a polypeptide, does not require absolute purity such as a homogenous preparation rather it represents an indication that the polypeptide is relatively purer than in the natural environment Generally, a purified polypeptide is substantially free of other proteins, lipids, carbohydrates, or other materials with which it is naturally associated, preferably at a functionally significant level for example at least 85% pure, more preferably at least 95% pure, most preferably at least 99% pure. A skilled artisan can purify a polypeptide comprising a glycosyltransferase using standard techniques for protein purification. A substantially pure polypeptide comprising a glycosyltransferase will yield a single major band on a non-reducing polyacrylamide gel. The purity of the glycosyltransferase can also be determined by amino-terminal amino acid sequence analysis.

A polypeptide used in the method may be chemically synthesized in whole or in part using techniques that are well-known in the art. Alternatively, methods are well known to the skilled artisan to construct expression vectors containing the native or mutated glycosyltransferase coding sequence and appropriate transcriptional/translational control signals. These methods include in vitro recombinant DNA techniques, synthetic techniques, and in vivo recombination/genetic recombination. See for example the techniques described in Sambrook et al. Molecular Cloning: A Laboratory Manual, 2nd Edition, Cold Spring Harbor Laboratory press (1989)), and other laboratory textbooks. (See also Sarker et al, Glycoconjugate 1. 7:380, 1990; Sarker et al, Proc. Natl. Acad, Sci. USA 88:234-238, 1991, Sarker et al, Glycoconjugate J. 11: 204-209, 1994; Hull et al, Biochem Biophys Res Commun 176:608, 1991 and Pownall et al, Genomics 12:699-704, 1992).

Preferably the polypeptide comprises a glycosyltransferase enzyme or part thereof having a mutation in the part of the enzyme which is involved in attachment to bacterial membranes. In a preferred embodiment the polypeptide comprises a glycosyltransferase enzyme or part thereof having a deletion at or around the C-terminus. In particular, such a deletion may serve to reduce the proportion of basic and/or hydrophobic and/or aromatic residues. The polypeptide may, for example, be missing the C-terminal 25 residues.

Preferably the polypeptide comprises one or more mutations which serve to reduce or eliminate aggregation of the polypeptide. For example, the polypeptide may comprise one or more mutations (e.g. substitutions or deletions) of cysteine residues.

Crystals may be grown from an aqueous solution containing the purified glycosyltransferase polypeptide by a variety of conventional processes. These processes include batch, liquid, bridge, dialysis, vapor diffusion, and hanging drop methods. (See for example, McPherson, 1982 John Wiley, New York; McPherson, 1990, Eur. J. Biochem. 189: 1-23; Webber. 1991, Adv. Protein Chem. 41:1-36). Generally, the native crystals of the invention are grown by adding precipitants to the concentrated solution of the glycosyltransferase polypeptide. The precipitants are added at a concentration just below that necessary to precipitate the protein. Water is removed by controlled evaporation to produce precipitating conditions, which are maintained until crystal growth ceases.

Derivative crystals of the invention can be obtained by soaking native crystals in a solution containing salts of heavy metal atoms. A complex of the invention can be obtained by soaking a native crystal in a solution containing a compound that binds the polypeptide, or they can be obtained by co-crystallizing the polypeptide in the presence of one or more compounds. In order to obtain co-crystals with a compound which binds deep within the tertiary structure of the polypeptide (for example UDP-2FGal is almost entirely buried by LgtC when bound) it is necessary to use the second method.

In a preferred embodiment, the polypeptide is cocrystallised with a compound which stabilises the polypeptide. For example, the compound may stabilise one or both of the loops made up of residues 75-80 and 246-251. In a highly preferred embodiment the polypeptide is cocrystallised with an inert analogue of the sugar donor, for example UDP 2-deoxy-2-fluorogalactose.

Once the crystal is grown it can be placed in a glass capillary tube and mounted onto a holding device connected to an X-ray generator and an X-ray detection device. Collection of X-ray diffraction patterns are well documented by those skilled in the art (See for example, Ducruix and Geige, 1992, IRL Press, Oxford, England). A beam of X-rays enter the crystal and diffract from the crystal. An X-ray detection device can be utilized to record the diffraction patterns emanating from the crystal. Suitable devices include the Marr 345 imaging plate detector system with an RU200 rotating anode generator.

Methods for obtaining the three dimensional structure of the crystalline form of a molecule or complex are described herein and known to those skilled in the art (see Ducruix and Geige 1992, IRL Press, Oxford, England). Generally, the x-ray crystal structure is given by the diffraction patterns. Each diffraction pattern reflection is characterized as a vector and the data collected at this stage determines the amplitude of each vector. The phases of the vectors may be determined by the isomorphous replacement method where heavy atoms soaked into the crystal are used as reference points in the X-ray analysis (see for example, Otwinowski, 1991, Daresbury, United Kingdom, 80-86). The phases of the vectors may also be determined by molecular replacement (see for example, Naraza, 1994, Proteins 11:281-296). The amplitudes and phases of vectors from the crystalline form of a glycosyltransferase determined in accordance with these methods can be used to analyze other related crystalline polypeptides.

The unit cell dimensions and symmetry, and vector amplitude and phase information can be used in a Fourier transform function to calculate the electron density in the unit cell i.e. to generate an experimental electron density map. This may be accomplished using the PHASES package (Furey, 1990). Amino acid sequence structures are fit to the experimental electron density map (i.e. model building) using computer programs (e.g. Jones, T A. et al, Acta Crystallogr A47, 100-119, 1991). This structure can also be used to calculate a theoretical electron density map. The theoretical and experimental electron density maps can be compared and the agreement between the maps can be described by a parameter referred to as R-factor. A high degree of overlap in the maps is represented by a low value R-factor. The R-factor can be minimized by using computer programs that refine the structure to achieve agreement between the theoretical and observed electron density map. For example, the XPLOR program, developed by Brunger (1992, Nature 355:472-475) can be used for model refinement.

A three dimensional structure of the molecule or complex may be described by atoms that fit the theoretical electron density characterized by a minimum R value. Files can be created for the structure that defines each atom by co-ordinates in three dimensions.

Model

A crystal structure of the present invention may be used to make a model of the glycosyltransferase or a part thereof, (e.g. a ligand-binding pocket). A model may, for example, be a structural model or a computer model. A model may represent the secondary, tertiary and/or quaternary structure of the glycosyltransferase. The model itself may be in two or three dimensions. It is possible for a computer model to be in three dimensions despite the constraints imposed by a conventional computer screen, if it is possible to scroll along at least a pair of axes, causing “rotation” of the image.

As used herein, the term “modelling” includes the quantitative and qualitative analysis of molecular structure and/or function based on atomic structural information and interaction models. The term “modelling” includes conventional numeric-based molecular dynamic and energy minimization models, interactive computer graphic models, modified molecular mechanics models, distance geometry and other structure-based constraint models.

Preferably, modelling is performed using a computer and may be further optimized using known methods. This is called modelling optimisation.

The three dimensional structure of a new crystal may be modelled using molecular replacement The term “molecular replacement” refers to a method that involves generating a preliminary model of a molecule or complex whose structural co-ordinates are unknown, by orienting and positioning a molecule whose structural co-ordinates are known within the unit cell of the unknown crystal, so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. Lattman, E., “Use of the Rotation and Translation Functions”, in Methods in Enzymology, 115, pp. 55-77 (1985); M. G. Rossmann, ed., “The Molecular Replacement Method”, Int. Sci. Rev. Ser., No. 13, Gordon & Breach, New York, (1972).

Commonly used computer software packages for molecular replacement are X-PLOR (Brunger 1992, Nature 355: 472-475), AMORE (Navaza, 1994, Acta Crystallogr. A50:157-163), the CCP4 package (Collaborative Computational Project, Number 4, “The CCP4 Suite: Programs for Protein Crystallography”, Acta Cryst., Vol. D50, pp. 760-763, 1994), the MERLOT package (P. M. D. Fitzgerald, J. Appl. Cryst., Vol. 21, pp. 273-278, 1988) and XTALVIEW (McCree et al (1992) J. Mol. Graphics 10: 44-46. It is preferable that the resulting structure not exhibit a root-mean-square deviation of more than 3 Å.

Molecular replacement computer programs generally involve the following steps: (1) determining the number of molecules in the unit cell and defining the angles between them (self rotation function); (2) rotating the known structure against diffraction data to define the orientation of the molecules in the unit cell (rotation function); (3) translating the known structure in three dimensions to correctly position the molecules in the unit cell (translation function); (4) determining the phases of the X-ray diffraction data and calculating an R-factor calculated from the reference data set and from the new data wherein an R-factor between 30-50% indicates that the orientations of the atoms in the unit cell have been reasonably determined by the method; and (5) optionally, decreasing the R-factor to about 20% by refining the new electron density map using iterative refinement techniques known to those skilled in the art (refinement).

The quality of the model may be analysed using a program such as PROCHECK or 3D-Profiler [Laskowski et al 1993 J. Appl. Cryst. 26:283-291; Luthy R. et al, Nature 356: 83-85, 1992; and Bowie, J. U. et al Science 253: 164-170, 1991]. Once any irregularities have been resolved, the entire structure may be further refined.

Other molecular modelling techniques may also be employed in accordance with this invention. See, e.g., Cohen, N. C. et al, “Molecular Modelling Software and Methods for Medicinal Chemistry”, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Computer format of Crystals/Models

Information derivable from the crystal of the present invention (for example the structural coordinates) and/or the model of the present invention may be provided in a computer-readable format.

Therefore, the invention provides a computer readable medium or a machine readable storage medium which comprises the structural co-ordinates of a retaining glycosyltransferase including all or any parts of the glycosyltransferase (e.g ligand-binding pockets), one or more ligands including substrates, for example, acceptor molecules including portions thereof, or donor molecules including portions thereof. Such storage medium or storage medium encoded with these data are capable of displaying on a computer screen or similar viewing device, a three-dimensional graphical representation of a molecule or molecular complex which comprises the enzyme or ligand binding pockets or similarly shaped homologous enzymes or ligand binding pockets. Thus, the invention also provides computerized representations of a crystal of the invention, including any electronic, magnetic, or electromagnetic storage forms of the data needed to define the structures such that the data will be computer readable for purposes of display and/or manipulation.

In an aspect the invention provides a computer for producing a three-dimensional representation of a molecule or molecular complex, wherein said molecule or molecular complex comprises a retaining glycosyltransferase or ligand binding pocket thereof defined by structural coordinates of retaining glycosyltransferase amino acids or a ligand binding pocket thereof, or comprises structural coordinates of atoms of a ligand in particular a substrate (e.g. an acceptor or donor molecule), or a three-dimensional representation of a homologue of said molecule or molecular complex, wherein said computer comprises:

(a) a machine-readable data storage medium comprising a data storage material encoded with machine readable data wherein said data comprises the structural coordinates of a retaining glycosyltransferase amino acids according to Table 4, 5, or 6 or a ligand binding pocket thereof, or an acceptor or donor molecule according to Table 4, 5, or 6;

(b) a working memory for storing instructions for processing said machine-readable data;

(c) a central-processing unit coupled to said working memory and to said machine-readable data storage medium for processing said machine readable data into said three-dimensional representation; and

(d) a display coupled to said central-processing unit for displaying said three-dimensional representation.

A homologue may comprise a glycosyltransferase or ligand binding pocket thereof, or acceptor or donor molecule that has a root mean square deviation from the backbone atoms of not more than 1.5 angstroms.

The invention also provides a computer for determining at least a portion of the structural coordinates corresponding to an X-ray diffraction pattern of a molecule or molecular complex wherein said computer comprises:

(a) a machine-readable data storage medium comprising a data storage material encoded with machine readable data wherein said data comprises the structural coordinates according to Table 4, 5, or 6;

(b) a machine-readable data storage medium comprising a data storage material encoded with machine readable data wherein said data comprises an X-ray diffraction pattern of said molecule or molecular complex;

(c) a working memory for storing instructions for processing said machine-readable data of (a) and (b);

(d) a central-processing unit coupled to said working memory and to said machine-readable data storage medium of (a) and (b) for performing a Fourier transform of the machine readable data of (a) and for processing said machine readable data of (b) into structural coordinates; and

(e) a display coupled to said central-processing unit for displaying said structural coordinates of said molecule or molecular complex.

The invention also contemplates a computer programmed with a model of a ligand binding pocket according to the invention; a machine-readable data-storage medium on which has been stored in machine-readable form a model of a ligand binding pocket of a glycosyltransferase; and the use of a model as input to a computer programmed for drug design and/or database searching and/or molecular graphic imaging in order to identify new ligands or modulators for glycosyltransferases.

Structural Determinations

The present invention also provides a method for determining the secondary and/or tertiary structures of a polypeptide by using a crystal, or a model according to the present invention. The polypeptide may be any polypeptide for which the secondary and or tertiary structure is uncharacterised or incompletely characterised. In a preferred embodiment the polypeptide shares (or is predicted to share) some structural or functional homology to the glycosyltransferase of the crystal. For example, the polypeptide may show a degree of structural homology over some or all parts of the primary amino acid sequence. For example the polypeptide may have one or more pockets which shows homology with a glycosyltransferase pocket (Kapitonov and Yu (1999) Glycobiology 9(10): 961-978).

Two polypeptides are considered to show substantial structural homology when the two peptide sequences, when optimally aligned (such as by the programs GAP or BESTFIT using default gap) share at least 40%, 50%, 60%, 65%, 70%, 75%, 80%, or 85% sequence identity, preferably at least 90 percent sequence identity, more preferably at least 95 percent sequence identity or more. Preferably, residue positions which are not identical differ by conservative amino acid substitutions. For example, the substitution of amino acids having similar chemical properties such as charge or polarity are not likely to affect the properties of a protein. Examples include glutamine for asparagine or glutamic acid for aspartic acid.

The polypeptide may be a glycosyltransferase with a different specificity for a ligand or portion thereof including a sugar residue, donor molecule or acceptor molecule. The polypeptide may be a glycosyltransferase which requires a different metal cofactor. Alternatively (or in addition) the polypeptide may be a glycosyltransferase enzyme from a different species.

The polypeptide may be a mutant of the wild-type glycosyltransferase. A mutant may arise naturally, or may be made artificially (for example using molecular biology techniques). The mutant may also not be “made” at all in the conventional sense, but merely tested theoretically using the model of the present invention. A mutant may or may not be functional.

Thus, using a model of the present invention, the effect of a particular mutation on the overall two and/or three dimensional structure of the glycosyltransferase and/or the interaction between the enzyme and a ligand can be investigated.

Alternatively, the polypeptide may perform an analogous function or be suspected to show a similar catalytic mechanism to the glycosyltransferase enzyme. For example the polypeptide may remove, transport, or add on a sugar residue. If the glycosyltransferase of the crystal is a retaining glycosyltransferase, the polypeptide under investigation may be known or suspected to function via a double-displacement mechanism.

The polypeptide may also be the same as the polypeptide of the crystal, but in association with a different ligand (for example, donor molecule, acceptor molecule analogue, modulator or inhibitor) or cofactor. In this way it is possible to investigate the effect of altering the ligand or compound with which the polypeptide is associated on the structure of the LBP.

Secondary or tertiary structure may be determined by applying the structural coordinates of the crystal or model of the present invention to other data such as an amino acid sequence, X-ray crystallographic diffraction data, or nuclear magnetic resonance (NMR) data. Homology modeling, molecular replacement, and nuclear magnetic resonance methods using these other data sets are described below.

Homology modeling (also known as comparative modeling or knowledge-based modeling) methods develop a three dimensional model from a polypeptide sequence based on the structures of known proteins (i.e. the glycosyltransferase of the crystal). The method utilizes a computer model of the crystal of the present invention (the “known structure”), a computer representation of the amino acid sequence of the polypeptide with an unknown structure, and standard computer representations of the structures of amino acids. The method in particular comprises the steps of; (a) identifying structurally conserved and variable regions in the known structure; (b) aligning the amino acid sequences of the known structure and unknown structure (c) generating coordinates of main chain atoms and side chain atoms in structurally conserved and variable regions of the unknown structure based on the coordinates of the known structure thereby obtaining a homology model; and (d) refining the homology model to obtain a three dimensional structure for the unknown structure. This method is well known to those skilled in the art (Greer, 1985, Science 228, 1055; Bundell et al 1988, Eur. J. Biochem. 172, 513; Knighton et al., 1992, Science 258:130-135, http://biochem.vt.edu/courses/modeling/homology.htn). Computer programs that can be used in homology modelling are Quanta and the Homology module in the Insight II modelling package distributed by Molecular Simulations Inc, or MODELLER (Rockefeller University, www.iucr.ac.uk/sinris-top/logical/prg-modeller.html).

In step (a) of the homology modelling method, the known glycosyltransferase structure is examined to identify the structurally conserved regions (SCRs) from which an average structure, or framework, can be constructed for these regions of the protein. Variable regions (VRs), in which known structures may differ in conformation, also must be identified. SCRs generally correspond to the elements of secondary structure, such as alpha-helices and beta-sheets, and to ligand- and substrate-binding sites (e.g. acceptor and donor binding sites). The VRs usually lie on the surface of the proteins and form the loops where the main chain turns.

Many methods are available for sequence alignment of known structures and unknown structures. Sequence alignments generally are based on the dynamic programming algorithm of Needleman and Wunsch [J. Mol. Biol. 48: 442453, 1970]. Current methods include FASTA, Smith-Waterman, and BLASTP, with the BLASTP method differing from the other two in not allowing gaps. Scoring of alignments typically involves construction of a 20×20 matrix in which identical amino acids and those of similar character (i.e., conservative substitutions) may be scored higher than those of different character. Substitution schemes which may be used to score alignments include the scoring matrices PAM (Dayhoff et al., Meth. Enzymol. 91: 524-545, 1983), and BLOSUM (Henilcoff and Henikoff, Proc. Nat. Acad. Sci. USA 89: 10915-10919, 1992), and the matrices based on alignments derived from three-dimensional structures including that of Johnson and Overington (JO matrices) (J. Mol. Biol. 233: 716-738, 1993).

Alignment based solely on sequence may be used; however, other structural features also may be taken into account. In Quanta, multiple sequence alignment algorithms are available that may be used when aligning a sequence of the unknown with the known structures. Four scoring systems (i.e. sequence homology, secondary structure homology, residue accessibility homology, CA-CA distance homology) are available, each of which may be evaluated during an alignment so that relative statistical weights may be assigned.

When generating co-ordinates for the unknown structure, main chain atoms and side chain atoms, both in SCRs and VRs need to be modelled. A variety of approaches known to those skilled in the art may be used to assign coordinates to the unknown. In particular, the coordinates of the main chain atoms of SCRs will be transferred to the unknown structure. VRs correspond most often to the loops on the surface of the polypeptide and if a loop in the known structure is a good model for the unknown, then the main chain coordinates of the known structure may be copied. Side chain coordinates of SCRs and VRs are copied if the residue type in the unknown is identical to or very similar to that in the known structure. For other side chain coordinates, a side chain rotamer library may be used to define the side chain coordinates. When a good model for a loop cannot be found fragment databases may be searched for loops in other proteins that may provide a suitable model for the unknown. If desired, the loop may then be subjected to conformational searching to identify low energy conformers if desired.

Once a homology model has been generated it is analyzed to determine its correctness. A computer program available to assist in this analysis is the Protein Health module in Quanta which provides a variety of tests. Other programs that provide structure analysis along with output include PROCHECK and 3D-Profiler [Luthy R. et al, Nature 356: 83-85, 1992; and Bowie, J. U. et al, Science 253: 164-170, 1991]. Once any irregularities have been resolved, the entire structure may be further refined. Refinement may consist of energy minimization with restraints, especially for the SCRs. Restraints may be gradually removed for subsequent minimizations. Molecular dynamics may also be applied in conjunction with energy minimization.

Molecular replacement involves applying a known structure to solve the X-ray crystallographic data set of a polypeptide of unknown structure. The method can be used to define the phases describing the X-ray diffraction data of a polypeptide of unknown structure when only the amplitudes are known. Thus in an embodiment of the invention, a method is provided for determining three dimensional structures of polypeptides with unknown structure by applying the structural coordinates of the crystal of the present invention to provide an X-ray crystallographic data set for a polypeptide of unknown structure, and (b) determining a low energy conformation of the resulting structure.

The structural coordinates of the crystal of the present invention may be applied to nuclear magnetic resonance (NMR) data to determine the three dimensional structures of polypeptides with uncharacterised or incompletely characterised structure. (See for example, Wuthrich, 1986, John Wiley and Sons, New York: 176-199; Pflugrath et al., 1986, J. Molecular Biology 189: 383-386; Kline et al., 1986 J. Molecular Biology 189:377-382). While the secondary structure of a polypeptide may often be determined by NMR data, the spatial connections between individual pieces of secondary structure are not as readily determined. The structural co-ordinates of a polypeptide defined by X-ray crystallography can guide the NMR spectroscopist to an understanding of the spatial interactions between secondary structural elements in a polypeptide of related structure. Information on spatial interactions between secondary structural elements can greatly simplify Nuclear Overhauser Effect (NOE) data from two-dimensional NMR experiments. In addition, applying the structural co-ordinates after the determination of secondary structure by NMR techniques simplifies the assignment of NOE's relating to particular amino acids in the polypeptide sequence and does not greatly bias the NMR analysis of polypeptide structure.

In an embodiment, the invention relates to a method of determining three dimensional structures of polypeptides with unknown structures, by applying the structural coordinates of a crystal of the present invention to nuclear magnetic resonance (NMR) data of the unknown structure. This method comprises the steps of: (a) determining the secondary structure of an unknown structure using NMR data; and (b) simplifying the assignment of through-space interactions of amino acids. The term “through-space interactions” defines the orientation of the secondary structural elements in the three dimensional structure and the distances between amino acids from different portions of the amino acid sequence. The term “assignment” defines a method of analyzing NMR data and identifying which amino acids give rise to signals in the NMR spectrum.

Screening Method

The present invention also provides a method of screening for a ligand that associates with a ligand binding pocket and/or modulates the function of a glycosyltransferase, by using a crystal or a model according to the present invention. The method may involve investigating whether a test compound is capable of associating with or binding a ligand binding pocket.

As used herein, the term “test compound” refers to any compound which is potentially capable of associating with a ligand binding pocket and/or modulating the function of a glycosyltransferase. If, after testing, it is determined that a test compound does bind to a LBP, it is known as a “ligand”.

A “test compound” includes but is not limited to, a compound which may be obtainable from or produced by any suitable source, whether natural or not. The test compound may be designed or obtained from a library of compounds which may comprise peptides, as well as other compounds, such as small organic molecules and particularly new lead compounds. By way of example, the test compound may be a natural substance, a biological macromolecule, or an extract made from biological materials such as bacteria, fungi, or animal (particularly mammalian) cells or tissues, an organic or an inorganic molecule, a synthetic test compound, a semi-synthetic test compound, a carbohydrate, a monosaccharide, an oligosaccharide or polysaccharide, a glycolipid, a glycopeptide, a saponin, a heterocyclic compound, a structural or functional mimetic, a peptide, a peptidomimetic, a derivatised test compound, a peptide cleaved from a whole protein, or a peptides synthesised synthetically (such as, by way of example, either using a peptide synthesizer or by recombinant techniques or combinations thereof), a recombinant test compound, a natural or a non-natural test compound, a fusion protein or equivalent thereof and mutants, derivatives or combinations thereof.

The test compound may be screened as part of a library or a data base of molecules. Data bases which may be used include ACD (Molecular Designs Limited), NCI (National Cancer Institute), CCDC (Cambridge Crystallographic Data Center), CAST (Chemical Abstract Service), Derwent (Derwent Information Limited), Maybridge (Maybridge Chemical Company Ltd), Aldrich (Aldrich Chemical Company), DOCK (University of California in San Francisco), and the Directory of Natural Products (Chapman & Hall). Computer programs such as CONCORD (Tripos Associates) or DB-Converter (Molecular Simulations Limited) can be used to convert a data set represented in two dimensions to one represented in three dimensions.

Test compounds may be tested for their capacity to fit spatially into the glycosyltransferase LBP. As used herein, the term “fits spatially” means that the three-dimensional structure of the test compound is accommodated geometrically in a cavity or pocket of the glycosyltransferase LBP. The test compound can then be considered to be a ligand.

A favourable geometric fit occurs when the surface areas of the test compound are in close proximity with the surface area of the cavity or pocket without forming unfavorable interactions. A favourable complementary interaction occurs where the test compound interacts by hydrophobic, aromatic, ionic, dipolar, or hydrogen donating and accepting forces. Unfavourable interactions may be steric hindrance between atoms in the test compound and atoms in the binding site.

In an embodiment of the invention, a method is provided for identifying potential modulators of a glycosyltransferase function. The method utilizes the structural coordinates or model of a glycosyltransferase three dimensional structure, or binding pocket thereof. The method comprises the steps of (a) docking a computer representation of a test compound from a computer data base with a computer model of a ligand binding pocket of a glycosyltransferase; (b) determining a conformation of a complex between the test compound and binding pocket with a favourable geometric fit or favorable complementary interactions; and (c) identifying test compounds that best fit the glycosyltransferase ligand binding pocket as potential modulators of glycosyltransferase function. The initial glycosyltransferase structure may or may not have ligands including substrates bound to it. A favourable complementary interaction occurs where a compound in a compound-glycosyltransferase complex interacts by hydrophobic, ionic, or hydrogen donating and accepting forces, with the active-site or binding pocket of a glycosyltransferase without forming unfavorable interactions.

If a model of the present invention is a computer model, the test compounds may be positioned in an LBP through computational docking. If, on the other hand, the model of the present invention is a structural model, the test compounds may be positioned in the LBP by, for example, manual docking.

As used herein the term “docking” refers to a process of placing a compound in close proximity with a glycosyltransferase LBP, or a process of finding low energy conformations of a test compound/glycosyltransferase complex.

A screening method of the present invention may comprise the following steps:

(i) generating a computer model of a glycosyltransferase or a ligand binding pocket thereof using a crystal according to the invention;

(ii) docking a computer representation of a test compound with the computer model;

(iii) analysing the fit of the compound in the glycosyltransferase or ligand binding pocket.

The method may be applied to a plurality of test compounds, to identify those that best fit the enzyme or ligand binding pocket.

In an aspect of the invention a method is provided comprising the following steps:

(a) docking a computer representation of a structure of a test compound into a computer representation of a ligand binding pocket of a glycosyltransferase defined in accordance with the invention using a computer program, or by interactively moving the representation of the test compound into the representation of the binding pocket;

(b) characterizing the geometry and the complementary interactions formed between the atoms of the ligand binding pocket and the compound; optionally

(c) searching libraries for molecular fragments which can fit into the empty space between the compound and ligand binding pocket and can be linked to the compound; and

(d) linking the fragments found in (c) to the compound and evaluating the new modified compound.

In an embodiment of the invention a method is provided which comprises the following steps:

(a) docking a computer representation of a test compound from a computer data base with a computer representation of a selected site (e.g. an inhibitor binding pocket) on a glycosyltransferase structure or model defined in accordance with the invention to obtain a complex;

(b) determining a conformation of the complex with a favourable geometric fit and favourable complementary interactions; and

(c) identifying test compounds that best fit the selected site as potential modulators of the glycosyltransferase.

The model used in the screening method may comprise the ligand-binding pocket of a glycosyltransferase enzyme either alone or in association with one or more ligands and/or cofactors. For example, the model may comprise the ligand-binding pocket in association with a donor molecule (or analogue thereof) and/or an acceptor molecule (or analogue thereof).

If the model comprises an unassociated ligand binding pocket, then the selected site under investigation may be the LBP itself. The test compound may, for example, mimic a known substrate for the enzyme (such as a donor or acceptor molecule) in order to interact with the LBP. The selected site may alternatively be another site on the enzyme (for example a site involved in attachment to the bacterial membrane).

If the model comprises an associated LBP, for example an LBP in association with a donor molecule or analogue thereof, the selected site may be the LBP or a site made up of the LBP and the complexed ligand, or a site on the ligand itself. The test compound may be investigated for its capacity to modulate the interaction with the associated molecule.

A test compound (or plurality of test compounds) may be selected on the basis of its similarity to a known ligand for the glycosyltransferase. For example, the screening method may comprise the following steps:

(i) generating a computer model of the LBP of a glycosyltransferase in complex with a ligand;

(ii) searching for a test compound with a similar three dimensional structure and/or similar chemical groups; and

(iii) evaluating the fit of the test compound in the LBP.

Searching may be carried out using a database of computer representations of potential compounds, using methods known in the art.

The present invention also provides a method for designing ligands for a glycosyltransferase. It is well known in the art to use a screening method as described above to identify a test compound with promising fit, but then to use this test compound as a starting point to design a ligand with improved fit to the model. Such techniques are known as “structure-based ligand design” (See Kuntz et al., 1994, Ace. Chem. Res. 27:117; Guida, 1994, Current Opinion in Struc. Biol. 4: 777; and Colman, 1994, Current Opinion in Struc. Biol. 4: 868, for reviews of structure-based drug design and identification;and Kuntz et al 1982, J. Mol. Biol. 162:269; Kuntz et al., 1994, Acc. Chem. Res. 27: 117; Meng et al., 1992, J. Compt. Chem. 13: 505; Bohm, 1994, J. Comp. Aided Molec. Design 8: 623 for methods of structure-based modulator design).

Examples of computer programs that may be used for structure-based ligand design are CAVEAT (Bartlett et al., 1989, in “Chemical and Biological Problems in Molecular Recognition”, Roberts, S. M. Ley, S. V.; Campbell, N. M. eds; Royal Society of Chemistry: Cambridge, pp 182-196); FLOG Miller et al., 1994, J. Comp. Aided Molec. Design 8:153); PRO Modulator (Clark et al., 1995 J. Comp. Aided Molec. Design 9:13); MCSS (Miranker and Karplus, 1991, Proteins: Structure, Function, and Genetics 8:195); and, GRID (Goodford, 1985, J. Med. Chem. 28:849).

The method may comprise the following steps:

(i) docking a model of a test compound with a model of a selected site;

(ii) identifying one or more groups on the test compound which may be modified to improve their fit in the selected site;

(iii) replacing one or more identified groups to produce a modified test compound model; and

(iv) docking the modified test compound model with the model of the selected site.

Evaluation of fit may comprise the following steps:

(a) mapping chemical features of a test compound such as by hydrogen bond donors or acceptors, hydrophobic/lipophilic sites, positively ionizable sites, or negatively ionizable sites; and

(b) adding geometric constraints to selected mapped features.

The fit of the modified test compound may then be evaluated using the same criteria.

The chemical modification of a group may either enhance or reduce hydrogen bonding interaction, charge interaction, hydrophobic interaction, Van Der Waals interaction or dipole interaction between the test compound and the key amino acid residue(s) of the selected site. Preferably the group modifications involve the addition, removal, or replacement of substituents onto the test compound such that the substituents are positioned to collide or to bind preferentially with one or more amino acid residues that correspond to the key amino acid residues of the selected site.

Identified groups in a test compound may be substituted with, for example, alkyl, alkoxy, hydroxyl, aryl, cycloalkyl, alkenyl, alkynyl, thiol, thioalkyl, thioaryl, amino, or halo groups. Generally, initial substitutions are conservative, i.e., the replacement group will have approximately the same size, shape, hydrophobicity and charge as the original group. It should, of course, be understood that components known in the art to alter conformation should be avoided.

If a modified test compound model has an improved fit, then it may bind to the selected site and be considered to be a “ligand”. Rational modification of groups may be made with the aid of libraries of molecular fragments which may be screened for their capacity to fit into the available space and to interact with the appropriate atoms. Databases of computer representations of libraries of chemical groups are available commercially, for this purpose.

The test compound may also be modified “in situ” (i.e. once docked into the potential binding site), enabling immediate evaluation of the effect of replacing selected groups. The computer representation of the test compound may be modified by deleting a chemical group or groups, or by adding a chemical group or groups. After each modification to a compound, the atoms of the modified compound and potential binding site can be shifted in conformation and the distance between the compound and the active site atoms may be scored on the basis of geometric fit and favourable complementary interactions between the molecules. This technique is described in detail in Molecular Simulations User Manual, 1995 in LUDI.

Examples of ligand building and/or searching computer include programs in the Molecular Simulations Package (Catalyst), ISIS/HOST, ISIS/BASE, and ISIS/DRAW (Molecular Designs Limited), and UNITY (Tripos Associates).

The “starting point” for rational ligand design may be a known ligand for the enzyme. For example, in order to identify potential modulators of the glycosyltransferase, a logical approach would be to start with a known ligand (for example a donor or acceptor molecule) to produce a molecule which mimics the binding of the ligand. Such a molecule may, for example, act as a competitive inhibitor for the true ligand, or may bind so strongly that the interaction (and inhibition) is effectively irreversible.

Such a method may comprise the following steps:

(i) generating a computer model of a LBP of a glycosyltransferase in complex with a ligand;

(ii) replacing one or more groups on the ligand model to produce a modified ligand; and

(iii) evaluating the fit of the modified ligand in the LBP.

The replacement groups could be selected and replaced using a compound construction program which replaces computer representations of chemical groups with groups from a computer database, where the representations of the compounds are defined by structural co-ordinates.

In an embodiment, a screening method is provided for identifying a ligand of a glycosyltransferase comprising the step of using the structural co-ordinates of a donor molecule or acceptor molecule or component thereof, defined in relation to its spatial association with a glycosyltransferase structure or a ligand binding pocket of the invention, to generate a compound that is capable of associating with the glycosyltransferase or ligand binding pocket.

The screening methods of the present invention may be used to identify compounds or entities that associate with a molecule that associates with a glycosyltransferase enzyme (for example, a substrate molecule).

In an embodiment of the invention, a screening method is provided for identifying a ligand of a glycosyltransferase comprising the step of using the structural co-ordinates of uridine, uracil, or UDP listed in Table 4, 5, or 6 to generate a compound for associating with the active site binding pocket of a glycosyltransferase as described herein. The following steps are employed in a particular method of the invention: (a) generating a computer representation of uridine, uracil, or UDP, defined by its structural coordinates listed in Table 4, 5, or 6; (b) searching for molecules in a data base that are structurally or chemically similar to the defined uridine, uracil, or UDP, using a searching computer program, or replacing portions of the compound with similar chemical structures from a database using a compound building computer program.

In another embodiment of the invention, a screening method is provided for identifying a ligand of a glycosyltransferase comprising the step of using the structural co-ordinates of UDP-Gal listed in Table 4, 5, or 6 to generate a compound for associating with the active site of a glycosyltransferase of the invention. The following steps are employed in a particular method of the invention: (a) generating a computer representation of UDP-Gal defined by its structural co-ordinates listed in Table 4, 5, or 6; and (b) searching for molecules in a data base that are structurally or chemically similar to the defined UDP-Gal using a searching computer program, or replacing portions of the compound with similar chemical structures from a database using a compound building computer program.

In another embodiment of the invention, a method is provided for designing potential inhibitors of a glycosyltransferase comprising the step of using the structural coordinates of a lactose molecule in Table 5, to generate a compound for associating with the active site of a glycosyltransferase.

The following steps are employed in a particular method of the invention: (a) generating a computer representation of a lactose acceptor defined by its structural coordinates listed in Table 4, 5, or 6; and (b) searching for molecules in a data base that are structurally or chemically similar to the defined lactose acceptor using a searching computer program, or replacing portions of the compound with similar chemical structures from a database using a compound building computer program.

The screening methods of the present invention may be used to identify compounds or entities that associate with a molecule that associates with a glycosyltransferase enzyme (for example, a donor or acceptor molecule).

Compounds and entities (e.g. ligands) of glycosyltransferases identified using the above-described methods may be prepared using methods described in standard reference sources utilized by those skilled in the art. For example, organic compounds may be prepared by organic synthetic methods described in references such as March, 1994, Advanced Organic Chemistry: Reactions, Mechanisms, and Structure, New York, McGraw Hill.

Test compounds and ligands which are identified using a model of the present invention can be screened in assays such as those well known in the art. Screening can be, for example, in vitro, in cell culture, and/or in vivo. Biological screening assays preferably centre on activity-based response models, binding assays (which measure how well a compound binds), and bacterial, yeast and animal cell lines (which measure the biological effect of a compound in a cell). The assays can be automated for high capacity-high throughput screening (HTS) in which large numbers of compounds can be tested to identify compounds with the desired activity. The biological assay, may also be an assay for the ligand binding activity of a compound that selectively binds to the ligand binding domain compared to other enzymes.

Ligands/Compounds/Modulators

The present invention provides a ligand or compound or entity identified by a screening method of the present invention. A ligand or compound may have been designed rationally by using a model according to the present invention. A ligand or compound identified using the screening methods of the invention specifically associate with a target compound. In the present invention the target compound may be the glycosyltransferase enzyme or a molecule that is capable of associating with the glycosyltransferase enzyme (for example a donor or acceptor molecule). In a preferred embodiment the ligand is capable of binding to the LBP of a glycosyltransferase.

A ligand or compound identified using a screening method of the invention may act as a “modulator”, i.e. a compound which affects the activity of a glycosyltransferase. A modulator may reduce, enhance or alter the biological function of a glycosyltransferase. For example a modulator may modulate the capacity of the enzyme to transfer a sugar from donor to acceptor. Alternatively, or in addition, it may modulate the capacity of the enzyme to attach to bacterial membranes. An alteration in biological function may be characterised by a change in specificity. For example, a modulator may cause the enzyme to accept a different acceptor or donor molecule, to transfer a different sugar, or to work with a different metal cofactor. In order to exert its function, the modulator commonly binds to the ligand binding pocket.

A “modulator” which is capable of reducing the biological function of the enzyme may also be known as an inhibitor. Preferably an inhibitor reduces or blocks the capacity of the enzyme to transfer a sugar from donor to acceptor. The inhibitor may mimic the binding of a donor or acceptor molecule, for example, it may be a donor or acceptor analogue. A donor or acceptor analogue may be designed by considering the interactions between the donor or acceptor molecule and the enzyme (for example by using information derivable from the crystal of the invention) and specifically altering one or more groups (as described above). Examples of donor and acceptor molecule analogues for LgtC are UDP-2Fgal and 4-deoxylactose respectively. Acceptor molecule analogues are also illustrated in Example 2.

In a highly preferred embodiment a modulator acts as an inhibitor of the glycosyltransferase and is capable of inhibiting lipooligosaccharide biosynthesis. Such an inhibitor may be useful as an antibiotic, because inhibition of LOS synthesis will prevent the bacterium from escaping detection by the human immune system by minicing human glycoproteins.

The present invention also provides a method for modulating the activity of a glycosyltransferase within a bacterial cell using a modulator according to the present invention. It would be possible to monitor the expression of LOS on the bacterial surface following such treatment by a number of methods known in the art (for example by detecting expression with an LOS-specific antibody).

In another preferred embodiment, the modulator is capable of causing or preventing oxidation of Cys 246. It is thought that oxidation of Cys 246 results in impaired donor and acceptor binding.

In another preferred embodiment, the modulator modulates the catalytic mechanism of the enzyme. For example it may affect the capacity of the side-chain oxygen of Gln 189 to act as a nucleophile in the double displacement mechanism.

A modulator may be an agonist, partial agonist, partial inverse agonist or antagonist of the glucosyltransferase.

As used herein, the term “agonist” means any ligand, which is capable of binding to a ligand binding pocket and which is capable of increasing a proportion of the enzyme that is in an active form, resulting in an increased biological response. The term includes partial agonists and inverse agonists.

As used herein, the term “partial agonist” means an agonist that is unable to evoke the maximal response of a biological system, even at a concentration sufficient to saturate the specific receptors.

As used herein, the term “partial inverse agonist” is an inverse agonist that evokes a submaximal response to a biological system, even at a concentration sufficient to saturate the specific receptors. At high concentrations, it will diminish the actions of a full inverse agonist.

The invention relates to a glycosyltransferase ligand binding pocket antagonist, wherein said ligand binding pocket is that defined by the amino acid structural coordinates described herein. For example the ligand may antagonise the inhibition of glycosyltransferase by an inhibitor.

As used herein, the term “antagonist” means any agent that reduces the action of another agent, such as an agonist. The antagonist may act at the same site as the agonist (competitive antagonism). The antagonistic action may result from a combination of the substance being antagonised (chemical antagonism) or the production of an opposite effect through a different receptor (functional antagonism or physiological antagonism) or as a consequence of competition for the binding site of an intermediate that links receptor activation to the effect observed (indirect antagonism).

As used herein, the term “competitive antagonism” refers to the competition between an agonist and an antagonist for a receptor that occurs when the binding of agonist and antagonist becomes mutually exclusive. This may be because the agonist and antagonist compete for the same binding site or combine with adjacent but overlapping sites. A third possibility is that different sites are involved but that they influence the receptor macromolecules in such a way that agonist and antagonist molecules cannot be bound at the same time. If the agonist and antagonist form only short lived combinations with the receptor so that equilibrium between agonist, antagonist and receptor is reached during the presence of the agonist, the antagonism will be surmountable over a wide range of concentrations. In contrast, some antagonists, when in close enough proximity to their binding site, may form a stable covalent bond with it and the antagonism becomes insurmountable when no spare receptors remain.

As mentioned above, an identified ligand or compound may act as a ligand model (for example, a template) for the development of other compounds. A modulator may be a mimetic of a ligand or ligand binding pocket A mimetic of a ligand (e.g an acceptor or donor molecule or part thereof) may compete with a natural ligand for a glycosyltransferase and antagonize a physiological effect of the enzyme in an animal. A mimetic of a ligand may be an organically synthesized compound. A mimetic of a ligand binding pocket, may be either a peptide or other biopharmaceutical (such as an organically synthesized compound) that specifically binds to a natural acceptor or donor molecule for a glycosyltransferase and antagonizes a physiological effect of the enzyme in an animal.

Once a ligand has been optimally selected or designed, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to a glycosyltransferase ligand binding pocket by the same computer methods described above. Preferably, positions for substitution are selected based on the predicted binding orientation of a ligand to a glycosyltransferase ligand binding pocket.

A modulator may be one or a variety of different sorts of molecule. For example, a modulator may be a peptide, member of random peptide libraries and combinatorial chemistry-derived molecular libraries, phosphopeptide (including members of random or partially degenerate, directed phosphopeptide libraries), a carbohydrate, a monosaccharide, an oligosaccharide or polysaccharide, a glycolipid, a glycopeptide, a saponin, a heterocyclic compound antibody, carbohydrate, nucleoside or nucleotide or part thereof, and small organic or inorganic molecule. A modulator may be an endogenous physiological compound, or it may be a natural or synthetic compound. The modulators of the present invention may be natural or synthetic. The term “modulator” also refers to a chemically modified ligand or compound.

A technique suitable for preparing a modulator will depend on its chemical nature. For example, peptides can be synthesized by solid phase techniques (Roberge J Y et al (1995) Science 269: 202-204) and automated synthesis may be achieved, for example, using the ABI 43 1 A Peptide Synthesizer (Perlin Elmer) in accordance with the instructions provided by the manufacturer. Once cleaved from the resin, the peptide may be purified by preparative high performance liquid chromatography (e.g., Creighton (1983) Proteins Structures and Molecular Principles, W H Freeman and Co, New York N.Y.). The composition of the synthetic peptides may be confirmed by amino acid analysis or sequencing (e.g., the Edman degradation procedure; Creighton, supra).

If a modulator is a nucleotide, or a polypeptide expressable therefrom, it may be synthesized, in whole or in part, using chemical methods well known in the art (see Caruthers M H et al (1980) Nuc Acids Res Symp Ser 215-23, Horn T et al (1980) Nuc Acids Res Symp Ser 225-232), or it may be prepared using recombinant techniques well known in the art.

Organic compounds may be prepared by organic synthetic methods described in references (e.g. March, 1994, Advanced Organic Chemistry: Reactions, Mechanisms, and Structure, New York, McGraw Hill).

The invention also relates to classes of modulators of glycosyltransferases based on the structure and shape of a ligand, in particular, a substrate including a donor molecule, or component thereof, or an acceptor molecule or component thereof, defined in relation to the ligand's spatial association with a glycosyltransferase structure of the invention or part thereof. Therefore, a modulator may comprise a ligand, in particular a donor molecule or an acceptor molecule, having the shape or structure, preferably the structural coordinates, of the ligand in the active site binding pocket of a reaction catalyzed by a glycosyltransferase.

A class of modulators of glycosyltransferases may comprise a compound containing a structure of uracil, uridine, ribose, pyrophosphate, or UDP, and having one or more, preferably all, of the structural coordinates of uracil, uridine, ribose, pyrophosphate, or UDP of Table 4, 5, or 6. In an embodiment, modulators are provided comprising the structure of UDP-Gal and having one or more, preferably all, of the structural co-ordinates of UDP-Gal of Table 4, 5, or 6. Functional groups in the uracil, uridine, ribose, pyrophosphate, UDP, or UDP-Gal modulators may be substituted with, for example, alkyl, alkoxy, hydroxyl, aryl, cycloalkyl, alkenyl, allynyl, thiol, thioalkyl, thioaryl, amino, or halo, or they may be modified using techniques known in the art. Substituents will be selected to optimize the activity of the modulator.

Modulators are also contemplated that comprise the structure of an acceptor molecule with the structural co-ordinates of lactose in Table 5 or 6. Functional groups in an acceptor structure may be substituted with, for example, alkyl, alkoxy, hydroxyl, aryl, cycloalkyl, alkenyl, alkynyl, thiol, thioalkyl, thioaryl, amino, or halo, or they may be modified using techniques known in the art. Substituents will b selected to optimize the activity of the modulator.

A class of modulators defined by the invention are compounds comprising the structural coordinates of UDP-Gal in the active site binding pocket of a reaction catalyzed by a glycosyltransferase. The UDP-Gal adopts a folded conformation in which the UDP moiety is bound in an extended manner and the galactose tucks back under the phosphates such that the plane of the galactose ring is almost parallel to the plane of the diphosphate (FIGS. 3).

Another class of modulators of the invention are compounds comprising a uridine diphosphate group having the structural co-ordinates of uridine diphosphate in the active site binding pocket of a reaction catalyzed by a glycosyltransferase.

Yet another class of modulators defined by the invention are compounds comprising the structural co-ordinates of lactose or an analogue thereof (4-deoxylactose, see also Example 2) in the active site binding pocket of a reaction catalyzed by a glycosyltransferase. The moieties of the lactose adopt a full chair conformation.

A class of modulators contemplated by the present invention are donor-acceptor complexes based on the spatial arrangement of a donor molecule and acceptor molecule in a transition state in a glycosyltransferase reaction. While not wishing to be bound by any particular theory, a retaining glycosyltransferase of the present invention may follow an SNi mechanism involving a direct displacement of the leaving group by the nucleophile, but from the front face of the sugar. Thus both the 4-hydroxyl of the lactose acceptor (the nucleophile) and the phosphate moiety of the UDP leaving group are located on the alpha face of the sugar. Reaction proceeds via a very dissociative (oxocarbenium ion-like) transition state. Precedent exists for this type of mechanism. (See J. Org Chem. (1994) 59, 1849; J. Org Chem. (1989) 54, 761;J. Org Chem. (1993), 58, 2822; see J. Amer Chem Soc (1980) 102, 2026:J. Amer. Chem. Soc. (1990) 10 113, 7958 re sugar systems). Based on this mechanism, the invention contemplates the following classes of modulators:

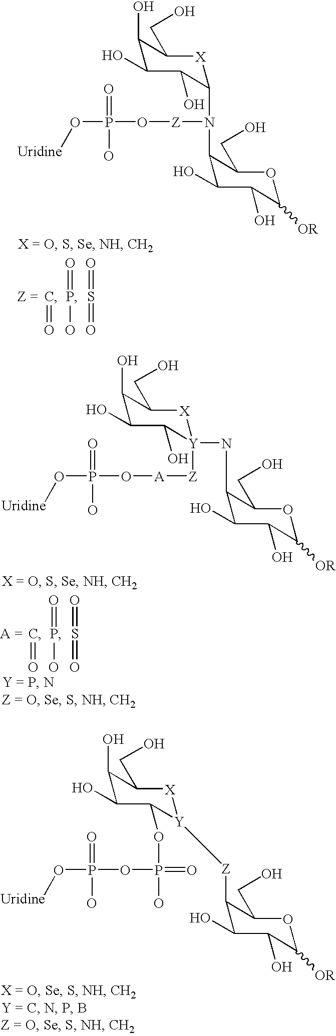

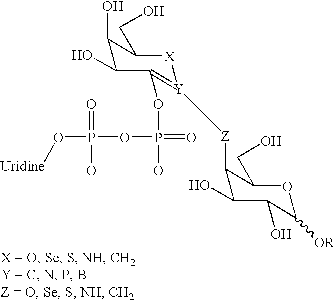

The invention contemplates all optical isomers and racemic forms of the modulators of the invention.

Pharmaceutical Composition

The present invention also provides the use of a ligand, in particular a modulator according to the invention, in the manufacture of a medicament to treat and/or prevent a disease in a mammalian patient. There is also provided a pharmaceutical composition comprising such a ligand or modulator and a method of treating and/or preventing a disease comprising the step of administering such a ligand or modulator or pharmaceutical composition to a mammalian patient.

The pharmaceutical compositions may be for human or animal usage in human and veterinary medicine and will typically comprise a pharmaceutically acceptable carrier, diluent, excipient, adjuvant or combination thereof.

Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise in addition to the carrier, excipient or diluent any suitable binder(s), lubricant(s), suspending agent(s), coating agent(s), and solubilising agent(s).

Preservatives, stabilizers, dyes and even flavouring agents may be provided in the pharmaceutical composition. Examples of preservatives include sodium benzoate, sorbic acid and esters of p-hydroxybenzoic acid Antioxidants and suspending agents may be also used.

The routes for admininstration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Where the pharmaceutical composition is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, gel, hydrogel, solution, cream, ointment or dusting powder, by use of a skin patch, orally in the form of tablets containing excipients such as starch or lactose or chalk, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

If the agent of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the agent; and/or by using infusion techniques.

For buccal or sublingual administration the compositions may be administered in the form of tablets or lozenges which can be formulated in a conventional manner.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate, and talc may be included.

Solid compositions of a similar type may also be employed as fillers in gelatin capsules. Preferred excipients in this regard include lactose, starch, cellulose, milk sugar, or high molecular weight polyethylene glycols. For aqueous suspensions and/or elixirs, the agent may be combined with various sweetening or flavouring agents, colouring matter or dyes, with emulsifying and/or suspending agents, and with diluents such as water, ethanol, propylene glycol and glycerin, and combinations thereof.

As indicated, a therapeutic agent of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™m) or 1,1,1,2,3,3,3-heptafluoropropane (HFA 227EA™), carbon dioxide or other suitable gas. In the case of a pressurised aerosol, the dosage unit may be determined by providing a valve to deliver a metered amount The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Therapeutic administration of polypeptide ligands (e.g. modulators) may also be accomplished using gene therapy. A nucleic acid including a promoter operatively linked to a heterologous polypeptide may be used to produce high-level expression of the polypeptide in cells transfected with the nucleic acid. DNA or isolated nucleic acids may be introduced into cells of a subject by conventional nucleic acid delivery systems. Suitable delivery systems include liposomes, naked DNA, and receptor-mediated delivery systems, and viral vectors such as retroviruses, herpes viruses, and adenoviruses.

The invention further provides a method of treating a mammal, the method comprising administering to a mammal a ligand (e.g. modulator) or pharmaceutical composition of the present invention.

Typically, a physician will determine the actual dosage which will be most suitable for an individual subject and it will vary with the age, weight and response of the particular patient and severity of the condition. The dosages below are exemplary of the average case. There can, of course, be individual instances where higher or lower dosage ranges are merited.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. By way of example, the pharmaceutical composition of the present invention may be administered in accordance with a regimen of 1 to 10 times per day, such as once or twice per day.

For oral and parenteral administration to human patients, the daily dosage level of the agent may be in single or divided doses.

Applications

As mentioned above, some glycosyltransferases are involved in the biosynthesis of bacterial lipooligosaccharide (LOS) which is thought to be essential for the pathogen to attach to host receptors and to evade the immune response (Kahler C M, Stephens D S, Crit Rev Microbiol 1998;24(4):281-334). A ligand or modulator may be able to modulate the activity of a glycosyltransferase within a bacterial cell. Hence a ligand or modulator according to the present invention may be capable of modulating LOS synthesis and therefore modulating bacterial attachment and/or recognition by the immune system.

Lipooligosaccharides (LOSs) are expressed on mucosal Gram-negative bacteria, including members of the genera Neisseria, Haemophilus, Bordetella, and Branhamella. They can also be expressed on some enteric bacteria such as Campylobacter jejuni and Campylobacter coli strains. LOSs share similar lipid A structures with an identical array of functional activities as LPSs. LOSs lack O-antigen units with the LOS oligosaccharide structures limited to 10 saccharide units. The LOS species of pathogenic Neisseria can play a major role in pathogenesis through enhancing the resistance of the organism to killing by normal human serum. Other distinguishing characteristics of LOS are the structural and antigenic similarity of some LOS species to human glycolipids and the potential for certain LOSs to be modified in vivo by host substances or secretions. These modifications of LOS in different environments of the host result in synthesis of new LOS structures that probably benefit the survival of the pathogen. The LOS of N. gonorrhoeae can act as a ligand of human receptors, promoting invasion of host cells.

Therefore, a ligand or modulator of the invention may be used to treat diseases caused by the following pathogenic organisms that have a LOS/LPS involvement in disease: Neisseria (meningitidis and gonnhorea) Haemophilus (influenzae and ducreyii), Branhanella (Moraxella), Canplyobacter, and Helicobacter. In a preferred embodiment the disease is associated with infection by a bacterium from the species Neisseria In a highly preferred embodiment the disease is associated with infection by Neisseria meningitidis, such diseases include, but are not limited to meningitis.

Meningococcal LOS is a critical virulence factor in N. meningitidis infections and is involved in many aspects of pathogenesis, including the colonization of the human nasopharynx, survival after bloodstream invasion, and the inflammation associated with the morbidity and mortality of meningococcemia and meningitis. Meningococcal LOS, which is a component of serogroup B meningococcal vaccines currently in clinical trials, has been proposed as a candidate for a new generation of meningococcal vaccines. (FEMS Immunol Med Microbiol 1996 Dec 1;16(2):105-15 Moran A P, Prendergast M M, Appelmelk B J)

LOS of pathogenic Neisseria spp. mimic the carbohydrate moieties of glycosphingolipids present on human cells. Such mimicry may serve to camouflage the bacterial surface from the host The LOS component is antigenically and/or chemically identical to lactoneoseries glycosphingolipids and can become sialylated in Neisseria gonorrhoeae when the bacterium is grown in the presence of cytidine 5′-monophospho-N-acetylneuraminic acid, the nucleotide sugar of sialic acid Strains of Neisseria meningitidis and Haemophilus influenzae also express similarly sialylated LPS. Sialylation of the LOS influences susceptibility to bactericidal antibody, may decrease or prevent phagocytosis, cause down-regulation of complement activation, and decrease adherence to neutrophils and the subsequent oxidative burst response. The core oligosaccharides of LPS of Campylobacter jejuni serotypes which are associated with the development of the neurological disorder, Guillain-Barre syndrome (GBS), exhibit mimicry of gangliosides. Cross-reactive antibodies between C. jejuni LPS and gangliosides are considered to play an important role in GBS pathogenesis. In contrast, the O-chain of a number of Helicobacter pylori strains exhibit mimicry of Lewis(x) and Lewis(y) blood group antigens. The role of this mimicry may play a role in bacterial camouflage, the induction of autoimmunity and immune suppression in H. pylori-associated disease. ( Crit Rev Microbiol 1996;22(3):139-8 Preston A, Mandrell R E, Gibson B W, Apicella M A)

Bordetella, does not use molecular mimicry but has either LOS or LPS as a critical virulence factor. (Infect Immun 2000 Dec;68(12):6720 Harvill E T, Preston A, Cotter P A, Allen A G, Maskell D J, Miller J F). Bordetella pertussis, Bordetella parapertussis, and Bordetella bronchiseptica are closely related subspecies that cause respiratory tract infections in humans and other mammals and express many similar virulence factors. Therefore, a ligand (e.g. modulator) of the invention may be used in preventing or treating diseases associated with Bordetella.

The following non-limiting examples are illustrative of the present invention:

### EXAMPLES

### Example 1

Material and Methods

Expression and Purification:

Recombinant LgtC-25 was over expressed in E. coli (AD202) as described previously (ref. 6). Briefly, the protein was first purified on a Q-sepharose fast flow column followed by a Superdex200 column (Pharmacia). Selenomethionyl LgtC was expressed in E. coli BL21 in minimal media supplemented with glucose and MgCl₂. When the cultures reached OD₆₀₀=0.6, selenomethionine (50 mg 1⁻¹) was added and at the same time the synthesis of methionine was down-regulated by the addition of 100 mg 1⁻¹ phenylalanine, threonine, lysine and 50 mg 1⁻¹ leucine, isoleucine, valine and proline (ref 41). After an additional 15 minutes, expression was induced with 0.5 mM IPTG after which the culture was grown for 6 hours. Selenomethionyl protein was purified following the same protocol with the exception that 5 mM DTT was included in all buffers. Incorporation of selenomethionine was confirmed by mass spectrometry. The structure was determined using a double cysteine mutant C128/174S from which more reproducible crystals could be produced than from the wildtype.

Site-directed Mutagenesis Via PCR.

All mutations were constructed in pCWlgtC-25 (ref. 6). A two stage PCR mutagenesis protocol was used. Two separate PCR reactions were performed, to generate two overlapping gene fragments, one of which contains the mutation. The primers were from the 5′ (primer 1) and (primer 2) 3′ ends of the gene as well as two internal primers. One internal primer contained the mutation and the other was chosen such that the 2 PCR products would overlap by 100 bp. These two products were gel purified, and then used as template for a third PCR reaction containing primers 1 and 2. This produced the fall length version of the gene with the mutation incorporated. Primers 1 and 2 contain BamHI and HindIII restriction sites which were used to subclone the final PCR product into pCW. Constructs were sequenced to verify the presence of only the mutation of interest.

Kinetic Assays

Synthesis of UDP-2Fgal was achieved essentially as described previously (ref. 19). Syntheses of 4′-deoxylactose, 6′-deoxylactose and galactosyl β-1,6-lactose will be described elsewhere. Kinetic studies were performed at 30° C. in 20 mM HEPES, pH 7.5 containing 0.1% bovine serum albumin, 50 mM KCl, 5 mM MnCl₂ and 5 mM DTT. The reaction was monitored via a continuous coupled assay similar to that described by Gosselin et al (ref. 42), in which UDP release is coupled to the oxidation of NADH (λ=340 nm, ε=6.22 nM⁻¹cm⁻¹). The change in absorbance was measured by means of a UNICAM 8700 UV-Vis spectrophotometer equipped with a circulating water bath. Kinetic parameters were calculated by direct fit of the initial rates to the respective equations using Grafit version 3.0 (ref.43).

Crystallization and Data Collection:

For crystallization 3 mM TCEP, 3 mM MnCl₂ and 5 mM UDP-2FGal was added to the protein (10 mg ml⁻¹) in 50 mM NH₄OAc, pH 7.0. The protein solution was further mixed in equal amounts with reservoir solution containing 50 mM NaOAc pH 5.0 and 5-20% PEG monomethylether 2000 and incubated on ice for 30 min before the solution was spun. Drops (6 μl) were allowed to equilibrate against the well solution as hanging drops and were streak seeded to induce crystallization. Orthorhombic LgtC crystals grow within a few hours of seeding and contain one molecule in the asymmetric unit (V_{M}=2.0 Å³ Da⁻¹) in space group P2₁2₁2₁ with unit cell dimensions a=39.79 Å b=76.05 Å c=86.84 Å.

A 2.0 Å MAD dataset, using four wavelengths, was collected at the Stanford Synchrotron Radiation Laboratory, beamline 1-5 using a Quantum Q4 CCD detector. For the deoxylactose complex, selenomethionyl crystals were soaked in 10 mM 4′-deoxylactose for 24 hours prior to data collection. Data were collected on a local Rigaka RU200 rotation anode equipped with OSMIC mirrors. All data were collected at 100K using mother liquor supplemented with 20% PEG 400 as cryoprotectant. All data was processed with DENZO and SCALEPACK (ref. 44). Statistics for data collection and processing are summarized in Table 2.

Structure Determination and Refinement:

The seven Se atom positions were determined using SOLVE (ref. 45). Phases and electron density maps were improved with DM (ref. 46). The initial density maps were of excellent quality and the model was easily built using XTALVIEW (ref. 47). The sequence differs from the published sequence (p96945) in three positions; an additional Gly was added at position 57, Ser 248 was exchanged for Pro and Gly 268 exchanged for Ala. These sequence differences were confirmed with DNA sequencing. The LgtC structure (with solvent, Mn and UDP-Gal removed) was used as the starting model for the 4-deoxylactose complex. Both complexes were refined with CNS 1.0 (ref. 48) where 5% of the data were flagged for the Rfree calculation. The parameters described by Engh and Huber (ref. 49) were used and the dictionaries for the substrates were generated by XPLO2D (ref. 50). All model building was performed using XTALVIEW. The quality of the models was analyzed with the program PROCHECK (ref. 51). Details of the refinement statistics are given in Table 2. Two regions with weak or no density have been excluded from the model, residues 218-221 between helix K and L, and the four C-terminal residues.

Structural Analysis and Figure Preparation:

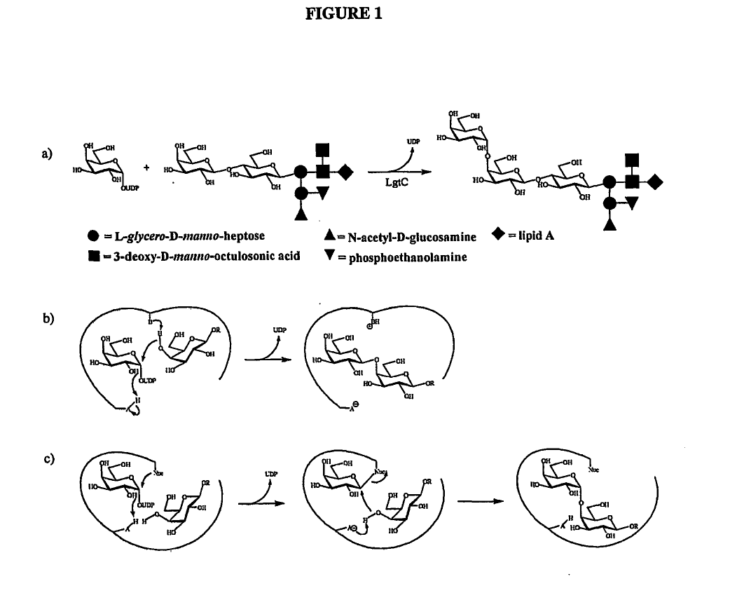

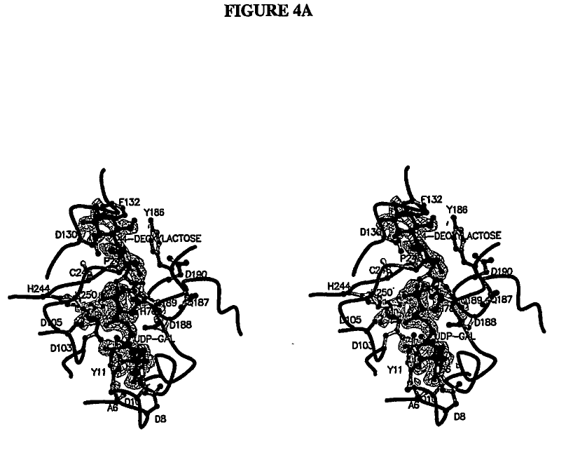

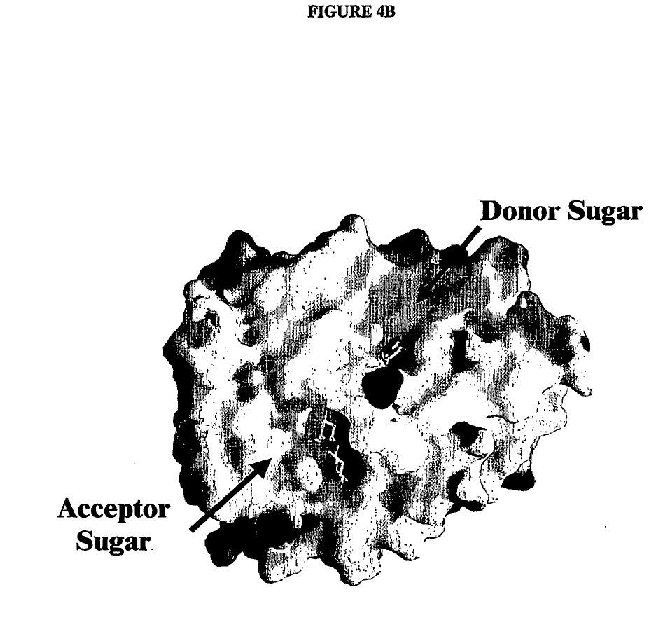

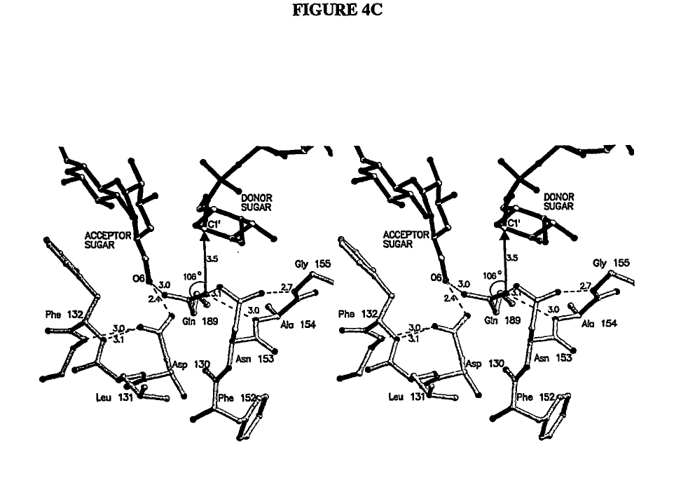

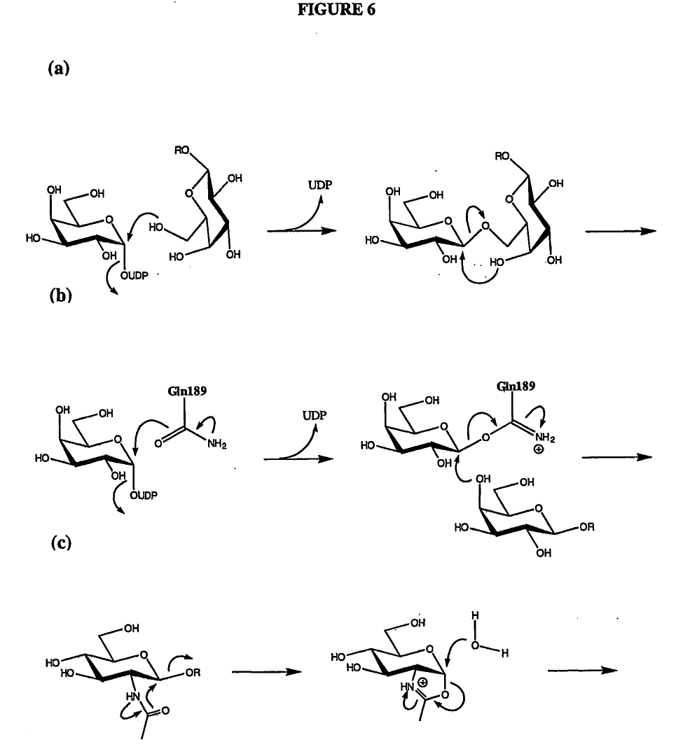

Surfaces were calculated with the CCP4 program AREAIMOL using a probe with a 1.4 Å radius . (ref. 34). Superpositions were done with the program TOP (ref. 18). FIGS. 1 and 6 were prepared with ChemDraw; FIGS. 3, 4c and 5 were prepared with MOLSCRIPT52 and rendered with Raster3D 53. FIG. 4b was prepared with GRASP (ref 54).

Results and Discussion

Overall Fold:

The galactosyl transferase structure determined here is that of a deletion mutant of LgtC missing the C-terminal 25 residues. This was necessary since the C-terminal 50 residues of LgtC have been proposed to be involved in attachment of LgtC (and other related sugar transferases) to the surface of the bacterial membrane (ref. 6), and the full length protein is less stable. As shown in FIG. 2 the deleted part of the enzyme is very rich in basic residues (Arg 287, Lys 292, Arg 293, Arg 297, Arg 299, Arg 300, Lys 301, Arg 305, Arg 308, Lys 309) which would be complementary to the negatively charged phospholipids in the membrane. There are also several hydrophobic and aromatic residues (Ala 283, Val 284, Met 288, Phe 289, Met 294, Leu 295, Trp 298. Leu 302, Ala 304, Phe 306, Leu 307, Ile 310, Tyr 311), which suggests that the C-terminus associates with the membrane via hydrophobic as well as electrostatic interactions. A sequence alignment of seven bacterial enzymes, all belonging to glycosyltransferase family 8, reveals that they are relatively conserved in terms of sequence and size in both the substrate binding and membrane association domains (FIG. 2). It was also necessary to replace several cysteine residues that were believed to be responsible for (reversible) aggregation of the protein in order to produce a protein that crystallized. X-ray quality crystals were then produced reliably only in the presence of an inert analogue of the sugar donor, namely UDP 2-deoxy-2-fluoro-α-D-galactopyranose (UDP-2FGal).

The structure determined is that of a monomer comprising 286 residues that form a large N-terminal α/β domain which contains the active site and a smaller helical C-terminal domain which mediates membrane attachment The overall fold is presented in FIG. 3. A central β-sheet forms the core of the α/β-domain. The sheet contains seven strands (β3, β2, β1, β4, β7, β6, β8) all of which are parallel with the exception of β7. The first 100 residues provide a nucleotide binding fold composed of four parallel strands. sandwiched between helices A and B on one side and helices C and D on the other. Helix C and the N-terminal part of helix D are both of 3₁₀ character. The remainder of the central β-sheet is flanked by four α-helices on each side. In addition, an antiparallel β-ribbon formed by β5 and β9 lies almost perpendicular to the sheet The substrate binding cleft is an extended, largely occluded groove that lies along the base of the central β-sheet.

The small C-terminal domain of LgtC, residues 248-282 (the last four residues are disordered), is mainly helical, with helix M and helix N (which is 3₁₀ in nature) forming a small pedestal that packs perpendicular to helices A and B of the nucleotide binding motif and to the β-ribbon (FIG. 3c).

A structural homology search using the TOP server (ref. 18) indicates that only the N-terminal nucleotide binding motif of LgtC shares significant structural similarity with other protein structures in the PDB. The remaining segment, residues 101-282, displays limited identity with other known folds. The top hit is the inverting glycosyltransferase bovine β-1,4-galactosyltransferase (ref. 14). Despite the low sequence identity (˜14%), superposition of the structure of this enzyme with that of LgtC yields a root mean square (rms) deviation of 2.1 Å on 83 common C-α atoms (with portions of elements β1,2,3,4,5,7,8 and helices A, D, H, J, K and M having the closest similarity). The glycosyl transferase SpsA from Bacillus subtilis (ref. 13) also has some structural similarity with LgtC (rms deviation of 2.1 Å on 76 common C-α atoms with portions of elements β1,2,3,5,7,9 and helices A, C, D, J and L having the closest similarity). A significant difference between LgtC and the inverting transferases (SpsA, B4G and phage T4-transferase) is that the donor UDP-Gal is bound in a much more shallow, solvent-exposed binding cleft in comparison to the deep, solvent-shielded cleft of LgtC. This may well reflect a greater need to exclude solvent from the active site of an enzyme that forms a reactive glycosyl-enzyme intermediate.

UDP-sugar Binding Mode

The structure of LgtC was solved in complex with Mn²⁺ and a non-cleavable analogue of the donor sugar, UDP-Gal in which the hydroxyl at the 2 position of the galactose has been substituted by a fluorine. The fluorine at the 2-position serves to inductively destabilize the oxacarbenium ion-like transition states for the reaction catalyzed, thereby slowing the reaction dramatically. Indeed linetic studies showed that no transfer occurs from UDP-2FGal, but that it acts as an excellent inhibitor, with a Kᵢ value of 2 μM (competitive with respect to UDP-Gal) as compared to the Kₘ value of 18 μM measured for UDP-Gal (Table 1). This fluorine substitution approach has been used previously to dramatically slow reaction rates in studies on glycosidases and on the mechanistically analogous glycogen phosphorylase¹⁰ and indeed UDP-2FGal has been synthesized previously and shown to inhibit two other galactosyltransferases¹⁹.

Most of the interactions between the enzyme and the nucleotide are formed by residues located at the C-terminus of β1 and the N-terminus of helix A. Two loops, from opposite sides of the groove, fold over the UDP-2FGal as a tight lid (FIGS. 3b, 4). The first loop, residues 75-80, is a part of the nucleotide binding motif and connects helix C with helix D. The second loop consists of residues 246-251 and is also part of a hinge between the N-terminal and the C-terminal domain. In the UDP-2FGal complex, the conformations of these two loops are stabilized primarily by interactions with the donor substrate in conjunction with additional intramolecular van der Waals (Vdw) interactions observed between His 78 and Pro 248. The structure indicates that these loops would likely be disordered or would adopt alternative conformations in the absence of the donor UDP-sugar. This may explain the inability to crystallize LgtC in the absence of UDP-2FGal.

The UDP-2FGal is almost entirely buried by the enzyme, leaving only 10 Å² or 1.5% of the molecule exposed to solvent (FIG. 4b). The donor sugar is highly ordered (Table 2) and adopts an unusual folded conformation in which the UDP moiety is bound in an extended manner but the galactose tucks back under the phosphates such that the plane of the galactose ring is almost parallel to the plane of the diphosphate (FIGS. 3, 4, 5). In other UDP-Gal complexes in the PDB, (for example in UDP-galactose 4-epimerase (ref. 20)), the UDP-Gal (or UDP-Glc) are bound in fully extended forms. As a measure of this conformation, a torsion angle calculated on PA and PB of the diphosphate and C1′ and C4′ of the galactose ring in LgtC is −10° compared to −160° in the epimerase structure. Interestingly, the conformation of the galactose-1-phosphate portion of the molecule is quite reminiscent of the conformation of glucose-1-phosphate or its analogues bound to glycogen phosphorylase (ref. 21). This is possibly quite relevant since glycogen phosphorylase is also a retaining glycosyltransferase.

The uridine diphosphate portion of UDP-2FGal binds in a cleft at the C-terminal end of the β-sheet while the uracil base stacks with conserved Tyr 11 (Phe in E. coli and Salmonella). The uracil carbonyl O4 forms a hydrogen bond with the ND2 group of Asn 10 while N3 of the base donates a hydrogen bond to OD1 of Asp 8. O2 of uracil is also within hydrogen bonding distance of the main chain nitrogen atom of Asp 8. The ribose ring adopts a C3-endo conformation in which 02 interacts with the carbonyl oxygen of Ala 6 and O3 with the main chain amide of Ile104. Both phosphates form hydrogen bonds with the protein, O2A with conserved Lys 250 (NZ) and O2B with Gly 247 (N) and His 78 (NE2).

The galactosyl moiety of the donor sugar is highly ordered within the LgtC active site (Table 2). The ring adopts a standard ⁴C₁ chair conformation similar to that of other UDP-galactose molecules in the PDB. O3′ forms hydrogen bonds to the side chain atoms of the invariant residues Asp 103 and Arg 86. O4′ and O6′ both hydrogen bond with the side chain carboxylate of the conserved Asp 188 indicating an important role for this residue in binding and probably in catalysis also. Such bidentate hydrogen bonding of a carboxyl group with vicinal hydroxyl groups on an active site sugar is well known, as in cyclodextrin glycosyltransferases and α-amylases (both family 13 ‘hydrolases’) where an aspartic acid residue bridges O2 and O3 of the substrate (ref. 22,23). F2 engages in only very weak interactions with a single active site residue (Asn 153), thereby possibly explaining why the binding constants of UDP-2Fgal and UDP-Gal are so similar. However, as a consequence of the folded conformation of the UDP-sugar, it engages in a relatively short interaction with an oxygen atom of the adjacent phosphate moiety. Presumably a hydroxyl group at the 2-position forms a hydrogen bond here, which likely becomes much stronger at the transition state as the glycosidic bond is cleaved and negative charge accumulates on the phosphate oxygens. This would stabilise the transition state, thus promoting catalysis. An additional hydrogen bond is formed between O6′ and the amide oxygen of the conserved Gln 189. There are also several Vdw interactions with the side chain atoms of Val 79, Thr 83, Gln 187 and Gln 189.

Manganese Binding

A “DXD” sequence motif is common to a wide range of glycosyltransferases, both in prokaryotes and eukaryotes, even though they may not share other sequence similarities (ref. 5,24,25). This motif has been proposed to be involved in the co-ordination of a divalent cation in the binding of the nucleotide sugar (ref. 26), though it may also show up in other contexts. Indeed, a number of mutagenesis studies have been carried out in various species on the conserved aspartate residues in the DXD sequence and all have found that enzymatic activity is completely abolished upon removal of the carboxylate, consistent with an important role in these cases (ref. 26-29). Interestingly, LgtC has four DXD motifs but only two are located within the active site of the enzyme. One is indeed shown to have important binding interactions with the metal ion while the role of the other is primarily in the binding of the acceptor sugar. Not surprisingly, these are also the only two served DXD motifs amongst the members of family 8. Based on these observations, it is clear that a DXD sequence is not always indicative of a metal binding site in glycosyltransferases and therefore should not be used as such. However, it is interesting to note that on the basis of the DXD sequence, an interesting glycosyltransferase activity was identified in the Fringe protein and this was shown to be responsible for modulating the activity of Notch receptors (ref. 30,31).

Earlier work has shown that manganese is required for enzyme stability and activity in LgtC, as in other related glycosyltransferases (ref. 6,32). It has been presumed that the metal functions to stabilize the UDP leaving group during bond cleavage. In the structure described herein, a single well-ordered Mn²⁺ ion is observed coordinated by the two phosphate oxygens of UDP as well as by the side chain atoms of three protein residues conserved throughout the family 8 glycosyltransferases, His 244, Asp 103 and Asp 105 (the latter two forming the DXD motif; FIG. 4). Asp 103 provides one liganding side-chain oxygen and Asp 105 provides a bidentate interaction. The co-ordination of the Mn²⁺ ion exhibits standard octahedral geometry with characteristic ligand distances ranging from 2.2 to 2.4 Å³³. Consistent with studies on DXD motifs in other systems, when Asp 103 is mutated to Asn or Glu activities drop dramatically, the k_{cat} values of the mutant being 2400-fold and 3500-fold lower compared to the wild type (Table 1). The bidentate ligand, Asp 105, is also essential for full activity; when substituted by Asn or Glu the mutated LgtC expresses very poorly.

Acceptor Binding:

The acceptor analog, 4-deoxylactose, in which the nucleophilic hydroxyl at the 4′ position has been replaced by hydrogen, cannot function as a substrate for LgtC, but does still act as an inhibitor Kᵢ=16 mM with respect to lactose). Its binding is very similar to that of lactose (Kₘ=20 mM) indicating that interactions at the 4-position are not crucial for ground state binding. Highly ordered electron density for both donor and acceptor molecules is observed from data collected on LgtC/UDP-2FGal crystals into which the 4-deoxylactose was soaked. The non-reactive acceptor analogue is bound in a large open pocket on the C-terminal end of the α/β domain adjacent to the galactose moiety of the donor sugar. The pocket is formed by the loop between helices C and D, the domain hinge, helices F, I, J and K, (FIGS. 3, 4). The acceptor sugar is significantly more accessible to solvent than the donor, 141 Å² or 28% of the entire molecular surface. The non-reducing terminal galactose moiety of the lactose adopts a full chair conformation. A hydrogen bond is formed from O2 to a water molecule and from O6 to Asp 130 (OD2) and Gln 189 (NE2). Binding is also stabilised by Vdw interactions with the side chain atoms of Val 76, His 78, Tyr 186, Cys 246 and Gly 247. Mutation of Asp 130 to an alanine severely limits protein expression, perhaps reflecting the intricate structural role this amino acid plays, with hydrogen bonds to the side chain nitrogen of the conserved Asn 153 and to the main chain amide of Val 133 as well as to the lactose O6. A mutant Y186F, was also generated to address the possibility of a role for its OH group in binding or catalysis upon rotation of its side chain hydroxyl closer to the reactive centre. However, the mutation affects neither expression nor kinetic parameters (Table 1) suggesting that such an important role is unlikely though Vdw interactions of its ring atoms with the lactose are probably important.

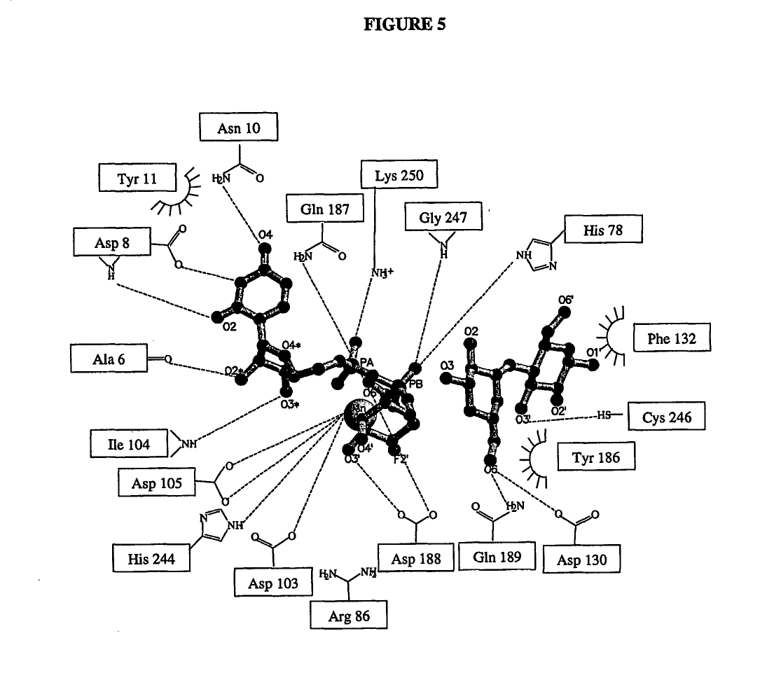

The reducing end glucose moiety of the lactose also adopts a full chair conformation, binding of the ring being stabilized by hydrophobic stacking interactions with Phe 132 and Vdw interactions with the side chain atoms of Pro 211 and Pro 248 (FIGS. 4, 5). Hydrogen bonds from O3′ to the Thr 212 hydroxyl and main chain nitrogen (via a water molecule) and a direct hydrogen bond to the side chain of Cys 246 are also observed. Biochemical studies have shown that full LgtC activity is dependent on the presence of reducing agents (ref. 6). The structure described herein clearly shows that no cysteine residues are at a suitable distance from each other to form a disulfide bridge. However, the structure does suggest that oxidation of Cys 246, located on one of the two loops that envelop the donor sugar and within hydrogen bonding distance to the acceptor sugar, could result in impaired donor and acceptor binding.

The LgtC/UDP-2FGal structure is minimally changed upon acceptor binding (r.m.s of 0.16 Å on 282 C-α atoms). All hydrogen bonds between the donor and the enzyme are maintained, with additional bonds observed from O2A of the phosphate, to Tyr 11 (OH) and the carbonyl of His 78 via a water molecule. Furthermore, in the donor/acceptor complex, the side chain of Cys 246 adopts a new conformation to form a hydrogen bond with the lactose O3′ atom. In the LgtC/UDP-2FGal complex an acetate ion is bound between sp 130 and Gln 189. Upon acceptor binding the acetate is displaced by the deoxylactose with the O6 atom forming hydrogen bonds to the side chain carboxylate of Asp 130 and the side chain amide of Gln 189.

Implications for Catalysis:

LgtC has been shown to follow an ordered bi-bi kinetic mechanism in which UDP-Gal binds first, followed by lactose. Bond rearrangement then occurs and product trisaccharide is released first, followed by UDP. The structure determined is completely consistent with this mechanism since the UDP-2Fgal is deeply buried by two loops that fold over the active site. Acceptor sugar is not required to form this complex, and indeed no significant changes in the structure of this complex are seen upon binding of 4-deoxylactose. Importantly the 2-fluorogalactose moiety is highly ordered, with multiple hydrogen bonds and Vdw interactions from conserved active site residues. Although the order of Mn²⁺ binding has not been determined kinetically in this enzyme, the structure suggests that the metal ion remains bound to the protein throughout, consistent with the fact that addition of exogenous Mn²⁺ is not essential for catalysis. Its location in the active site, coordinating to oxygens of the α- and β-phosphate moieties of UDP-2Fgal, is typical, and is consistent with a role of the cation as an acid catalyst.

Efficient catalysis of galactosyl transfer requires that water be excluded from the active site, or at least carefully controlled therein, in order to ensure that hydrolysis does not occur. The close packing and occluded nature of the donor sugar in the LgtC structure limits the binding of ordered water molecules in the active site (FIG. 4b). In the UDP-2Fgal structure, only a single water molecule is within 5 Å of the reactive center C1′ of the donor galactose (4.4 Å) and this water is displaced by the O2 hydroxyl of deoxylactose upon formation of the ternary complex. Within this complex, the reactive center C1′ atom is entirely buried by residues Ile 76, Asp 103, Asp 130, Asp 153, Ala 154, Gly 155, Tyr 186, Gln 189, His 244, Cys 246, Gly 247 and by the acceptor sugar (as calculated with CONTACT³⁴ using a 6 Å cutoff). The closest water molecule in this complex is 7.3 Å away from the anomeric C1′ atom. Thus the enzyme has apparently evolved to exclude water, as would be expected.

As noted earlier, the stereochemical outcome of the reaction catalyzed suggests, by analogy with retaining glycosidases, that a double-displacement mechanism via a glycosyl-enzyme intermediate is occurring. If this is indeed true, then a suitable nucleophile should be located close to the anomeric carbon (C1′) of the UDP-2Fgal, and on the correct (β) face to allow direct displacement of the UDP leaving group.

Analysis of the LgtC structure indicates that the only polar atoms within 5 Å of the reactive center C1′ come from either the acceptor sugar (the lactose hydroxyl O3 (3.2 Å) and hydroxymethyl O6 (4.6 Å)) or from the side chain oxygen atom of Gln 189 (3.5 Å), and the side chain nitrogen atom of Asn 153 (4.2 Å). However, of these only the lactose O6 and the side chain oxygen atom of Gln 189 are located on the β-face on a reasonable trajectory. In light of experience with retaining glycosidases, this is surprising, since the carboxylate side chain of an Asp or Glu residue might have been anticipated on that basis. The two choices were therefore considered and evaluated separately, as follows.

The possibility of the 6-hydroxyl of lactose functioning in this fashion was initially intriguing, as this would have involved the enzyme first forming a tightly bound intermediate galactosyl β-1,6-lactose species. After movement away of the released UDP the 4-hydroxyl of the lactose could then attack the anomeric center from the α-face, forming the desired Gal α-1,4-lactose product (FIG. 6a). A particularly attractive component of this mechanism is the fact that it inherently demands formation of a ternary complex prior to the generation of a reactive intermediate, thereby minimizing the possibility of unwanted hydrolysis. Less attractive is the fact that the intermediate would not itself be inherently reactive, being a simple glycoside. The mechanism was probed experimentally in two ways. Firstly 6-deoxylactose, in which the putatively nucleophilic 6-hydroxyl had been removed, was synthesized and shown not to function as a substrate, which would be consistent with this mechanism. However, neither did it act as an inhibitor, thereby rendering its inactivity as a substrate meaningless. It does, however, indicate that binding interactions at that position, probably primarily with Asp 130, are quite important. More definitive results suggesting that this was not the likely mechanism come from the finding that galactosyl β-1,6-lactose, synthesized chemically, does not function as a substrate when incubated with LgtC in the presence of UDP plus the usual buffer components. Therefore, unless binding of this potential intermediate is too slow to permit measurable turnover, this mechanism is unlikely.

The alternative mechanism, in which the oxygen of Gln 189 attacks at the anomeric center to form an imidic ester intermediate (FIG. 6b) is initially unattractive given the fact that amides are notoriously poor nucleophiles. However, the reaction is entirely precedented, even within the field of glycosidases, since a chemically equivalent intermediate has been demonstrated to form during catalysis by N-acetylhexosaminidases from glycosidase families 18 and 20 (see ref. 10,11 for reviews). In those cases the substrate's own amide functionality attacks to form an oxazolinium ion intermediate (FIG. 6c) that is charge stabilized by an invariant carboxylate side chain close to the substrate nitrogen atom. In LgtC the oxygen atom of the side chain amide is well-positioned to perform a nucleophilic attack on C1′ both in terms of distance (3.5 Å) and in terms of the direction and angle of attack: the Cδ-Oε1-C1′ angle is 106.2° in good agreement with ideal values (FIG. 4c)³⁵. This identical position is seen in the complex with UDP-2Fgal (r.m.s of 0.14 Å on the 9 atoms of the residue), as might be expected for a pre-organised nucleophile. Gln 189 is contained within the invariant D/EQD motif (Helix J) found in all family 8 retaining glycosyltransferases (FIG. 3) (ref. 5). The side chain amide of Glnl 89 is fully buried in the donor/acceptor complex, and is oriented through several hydrogen bonds to both sugar (donation of a hydrogen bond from Nε1 to O6 of the lactose) and conserved protein side chains (acceptance of a hydrogen bond from the side chain Nε2 of Asn 153 (itself an invariant residue found within the NAG motif in all family 8 glycosyltransferases) and the main-chain NH of Ala 154, FIG. 4c). Furthermore, charge stabilization could be provided by the nearby (4.0 Å) carboxylate side chain of Asp 130.

Interestingly a mechanism of this type has been hinted at previously for another glycosyltransferase, glycogen phosphorylase, on the basis of structures of complexes with a suspected transition state analogue inhibitor, deoxynojirimycin tetrazole (ref. 36), and of a ternary complex with a thiooligosaccharide plus phosphate (ref. 37). In both cases the group identified as being closest to the anomeric center of the sugar to be transferred, and in the best position to function as catalytic nucleophile, is the main chain oxygen of the backbone amide of His 377. Similar to LgtC, stabilization of the developing positive charge on the nitrogen in this case can be afforded by the nearby aspartate (Asp 307) located within 4 Å of the amide nitrogen. Given the parallels in the reaction catalysed and the similarity in 3-dimensional structure with other transferases (ref. 38,39) this could indicate similar roles for these two amide carbonyl groups of LgtC and glycogen phosphorylase.

This mechanistic hypothesis was tested in LgtC through mutagenesis and kinetic analysis of the resultant mutants. The mutant Q189A has a k_{cat} value equaling 3% of that of the wild type enzyme (based on k_{cat} values measured with varying UDP-Gal), and a very similar Kₘ value for UDP-Gal. Interestingly, the Kₘ value for lactose was considerably (6-7 fold) higher than that for the wild type enzyme, consistent with the presence of a hydrogen bond between Nε₁ of Gln 189 and 06 of lactose. Importantly this confirms that the activity measured is indeed that of the mutant, and not due to contaminating wild type enzyme (itself unlikely since considerable precautions were taking during purification, including the use of new column packing materials for purification of this mutant). However, this relatively high residual activity renders a role for Gln 189 as the catalytic nucleophile somewhat unlikely given the presumed crucial importance of such a residue. Indeed, equivalent mutations of catalytic nucleophiles in retaining glycosidases typically reduce k_{cat} values by at least 10⁵ fold (ref. 10,11). The possibility that the mutant catalyses hydrolysis rather than transfer as a consequence of water binding in the site vacated by the side chain of Gln 189 was evaluated by product analysis. Only transfer products were observed, not galactose. Possible ‘rescue’ of activity by added small molecules that could bind in the cavity created, as has been seen for retaining glycosidases (ref. 11,40) was also probed. No rate increases were observed with any of the added reagents (formate, acetate, formamide, azide, acetamide), although modeling suggests that binding of these molecules in the Q189A mutation may be sterically unfavorable. Another possibility that cannot be ruled out is that an adjacent residue substitutes for Gln 189 within this mutant, but with lower efficiency.

Given the fact that definitive evidence of a double displacement mechanism remains elusive, a third possibility, but one with only limited chemical precedent, is that the reaction proceeds via a front side S_{N}2-like attack, otherwise known as an S_{N}i mechanism. In this scenario, approach of the nucleophile (the reactive hydroxyl of the acceptor sugar) towards the reactive centre would occur from the same side from which the UDP leaving group would depart and reaction would most likely proceed via a highly dissociative (oxocarbenium ion-like) transition state. Such a mechanism has been proposed previously for glycogen phosphorylase (ref. 9), but no experimental support has yet been accumulated.

Conclusions:

Determination of this first three-dimensional structure of a retaining nucleotide sugar-dependent glycosyl transferase in a complex with analogues of both substrates for the enzyme provides unique insights into the structure and mechanism of this important class of enzymes. Partial commonality of fold with those of several inverting transferases suggest that common structural elements are employed in the construction of a glycosyl transfer site, irrespective of the stereochemical outcome of the reaction.

This is the first structure of a glycosyltransferase to provide any structural information about the donor and acceptor sugars, and the first crystal structure of a retaining transferase, and is invaluable for rational inhibitor design.

### Example 2

Synthesis of Alternate Acceptor Substrates:

2,2′,3,3′,4′,6,6′-Hepta-O-acetyl-α-lactosyl Bromide (1)

To a 0° C. solution of per-O-acetylated β-D-lactose (4.7 g, 6.87 mmol) in anhydrous CH₂Cl₂ (15 mL) under nitrogen was added 45% HBr/AcOH (5.3 mL). The reaction vessel was then sealed and the solution was allowed to stir at room temperature. After 2.5 h, the reaction mixture was poured into chilled water (80 mL) and diluted with CH₂Cl₂ (60 mL). Solid NaHCO₃ was added to neutralize the excess acid and the layers were separated. The aqueous layer was further extracted with CH₂Cl₂ (2×80 mL) and the combined organic extracts were washed with water (2×80 mL), aq. NaHCO₃ (50 mL) and brine (50 mL). Evaporation of the solvent under reduced pressure after drying over MgSO₄ yielded 1 (4.5 g, 93%) as a white brittle solid. ¹H NMR (CDCl₃, 400 MHz): δ 6.5 (d, 1 H, J_{1,2} 4.0 Hz, H-1), 5.53 (dd, 1 H, J_{3,2} 9.6, J_{3,4} 9.6 Hz, H-3), 5.33 (dd, 1 H, J_{4′,3′} 3.4, J_{4′,5′} 0.9 Hz, H-4′), 5.11 (dd, 1 H, J_{2′,3′} 10.4, J_{2′,1′} 7.9 Hz, H-2′), 4.94 (dd, 1 H, J_{3′,2′} 10.4, J_{3′3,4′}3.4 Hz, H-3′), 4.74 (dd, 1 H, J_{2,3} 9.6, J_{2,1} 4.0 Hz, H-2), 4.49 (d, 1 H, J_{1′,2′} 7.9 Hz, H-1′), 4.47 (dd, 1 H, J_{6a,6b} 12.0, J_{6a,5} 1.7 Hz, H-6a), 4.02-4.23 (m, 4 H, H-5, H-6b, H-6a′, H-6b′), 3.86 (ddd, 1 H, J_{5′,6b′} 7.3, J_{5′,6a′} 6.4, J_{5′,4′} 0.9 Hz, H-5′), 3.83 (dd, 1 H, J_{4,5} 9.8, J_{4,3} 9.6 Hz, H-4), 2.13, 2.10, 2.06, 2.04, 2.03, 2.02, 1.94 (s, 21 H, 7×OAc).

Benzyl 2,2′,3,3′,4′,6,6′-hepta-O-acetyl-β-lactoside (2)

1 (2.6 g, 3.69 mmol) was stirred in anhydrous CH₂Cl₂ (25 mL) containing 4 Å sieves under an atmosphere of nitrogen when benzyl alcohol (1.9 mL, 18.47 mmol) and AgCO₃ (2.0 g, 7.39 mmol) were added, along with a crystal of iodine. The reaction mixture was covered and stirred at room temperature overnight before it was filtered through Celite®. The filtrate was evaporated in vacuo and the residue was chromatographed over silica gel (PE:EtOAc, 3:2 to 1:1). Crystallization from PE/EtOAc yielded 2 (2.0 g, 75%) as a white solid. ¹H NMR (CDCl₃, 400 MHz): δ 7.22-7.35 (m, 5H, Ar), 5.32 (dd, 1 H, J_{4′,3′} 3.3, J_{4′,5′} 0.8 Hz, H-4′), 5.16 (dd, 1 H, J_{3,2} 9.3, J_{3,4} 9.2 Hz, H-3), 5.08 (dd, 1 H, J_{2′,3′} 10.4, J_{2′,1′} 7.9 Hz, H-2′), 4.99 (dd, 1 H, J_{2,3} 9.3, J_{2,1} 7.9 Hz, H-2), 4.93 (dd, 1 H, J_{3′,2′} 10.4, J_{3′,4′} 3.3 Hz, H-3′), 4.86 (d, 1 H, J 12.3 Hz, PhCH), 4.58 (d, 1 H, J 12.3 Hz, PhCH), 4.50 (d, 1 H, J_{1′,2′} 7.9 Hz, H-1′), 4.48 (d, 1 H, J_{1,2} 7.9 Hz, H-1), 4.48-4.52 (m, 1 H, H-6a), 4.00-4.15 (m, 3 H, H-6b, H-6a′, H-6b′), 3.84 (ddd, 1 H, J_{5′,6b′} 7.4, J_{5′,6a′} 6.3, J_{5′,4′} 0.8 Hz, h-5′), 3.79 (dd, 1 H, J_{4,5} 9.6, J_{4,3} 9.2 Hz, H-4), 3.56 (ddd, 1 H, J_{5,4} 9.6, J_{5,6b} 5.0, J_{5,6a} 2.0 Hz, H-5), 2.12, 2.11, 2.02, 2.01, 2.00, 1.98, 1.94 (s, 21H, 7×OAc).

Anal. Calcd. for C₃₃H₄₂O₁₈: C, 54.54; H, 5.83. Found: C, 54.50; H, 5.93.

Benzyl β-lactoside (3)

To a solution of 2 (1.24 g, 1.71 mmol) in anhydrous MeOH (30 mL) under nitrogen was added a catalytic amount of sodium methoxide until the pH of the solution was around 10. The reaction mixture was then stirred overnight at room temperature before it was neutralized with acidic Amberlyte® resin. After evaporation of the solvent, crystallization of the resulting residue from MeOH/EtOAc yielded 3 (0.64 g, 87%) as a white solid. ¹H NMR (D₂O, 400 MHz) selected data only: δ 7.30-7.60 (m, 5 H, Ar), 4.90 (d, 1 H, J 11.4 Hz, PhCH), 4.52 (d, 1 H, J_{1′,2′} 8.0 Hz, H-1′), 4.41 (d, 1 H, J_{1,2} 7.8 Hz, H-1), 3.95 (dd, 1 H, J_{6a,6b} 12.3, J_{6a,5} 2.1 Hz; H-6a), 3.88 (d, 1 H, J_{4′,3′} 3.3 Hz, H-4′), 3.32 (dd, 1 H, J_{4,5} 8.7, J_{4,3} 8.4 Hz, H-4).

Anal. Calcd. for C₁₉H₂₈O₁₁: C, 52.77; H, 6.53. Found: C, 52.47; H, 6.63.

Allyl 2,2′,3,3′,4′, 6,6′-hepta-O-acetyl-β-lactoside (4)

1 (0.5 g, 0.69 mmol) was stirred in anhydrous CH₂Cl₂ (5 mL) containing 4 Å sieves under an atmosphere of nitrogen when allyl alcohol (0.24 mL, 3.46 mmol) and Ag2CO₃ (0.4 g, 1.38 mmol) were added along with a crystal of iodine. The reaction was covered and stirred at room temperature for 9 h before it was filtered through Celite®. The filtrate was evaporated in vacuo and the residue was chromatographed over silica gel (PE:EtOAc, 3:2 to 1: 1) to yield 4 (0.35 g, 76%) as a white solid. ¹H NMR (CDCl₃, 400 MHz): δ 5.81 (m, 1 H, OCH₂CH=CH₂), 5.32 (dd, 1 H, J_{4′,3′} 3.4, J_{4′,5′} 0.9 Hz, H-4′), 5.23 (ddd, 1 H, Jₜᵣₐₙₛ 17.3, J 3.2, J_{gem} 1.6 Hz, OCH₂CH=CHₜᵣₐₙₛ), 5.17 (dd, 1 H, J_{3,2} 9.3, J_{3,4} 9.2 Hz, H-3), 5.17 (ddd, 1 H, J_{cis} 10.5, J 2.8, J_{gem} 1.6 Hz, OCH₂CH═CH_{cis}), 5.08 (dd, 1 H, J_{2′,3′} 10.4, J_{2′,1′} 7.9 Hz, H-2′), 4.93 (dd, 1 H, J_{3′,2′} 10.4, J_{3′,4′} 3.4 Hz, H-3′), 4.90 (dd, 1 H, J_{2,3} 9.3, J_{2,1} 7.9 Hz, H-2), 4.50 (d, 1 H, J_{1′,2′} 7.9 Hz, H-1′), 4.46 (d, 1 H, J_{1,2} 7.9 Hz, H-1), 4.44-4.50 (m, 1 H, H-6a), 4.27 (ddt, 1 H, J 13.2, J 4.9, J 1.5 Hz, OCR), 4.01-4.14 (m, 4 H, H-6b, H-6a′, H-6b′, OCH), 3.84 (ddd, 1 H, J_{5′,6b′} 7.2, J_{5′,6a′} 6.4, J_{5′,4′} 0.9 Hz, H-5′), 3.78 (dd, 1 H, J_{4,5} 9.7, J_{4,3} 9.2 Hz, H-4), 3.57 (ddd, 1 H, J_{5,4} 9.7, J_{5,6b} 5.0, J_{5,6a} 2.0 Hz, H-5), 2.13, 2.10, 2.03, 2.01, 1.94 (s, 21 H, 7×OAc).

Allyl β-lactoside (5)

4 (0.14 g, 0.21 mmol) was dissolved in anhydrous MeOH at 0° C. under an atmosphere of nitrogen when gaseous ammonia was bubbled into the solution. After 5 min, both the ammonia source and ice bath were removed and the reaction mixture was stirred overnight at room temperature. Evaporation of the solvent in vacuo followed by chromatography over silica gel (EtOAc:MeOH:H₂O, 15:4:1) yielded 5 (47.5 mg, 60%) as a white solid. ¹H NMR (D₂O, 400 MHz) selected data only: δ 5.95 (m, 1 H, OCH₂CH═CH₂), 5.35 (dd, 1 H, Jₜᵣₐₙₛ 17.3, J_{gem} 1.4 Hz, CH═CHₜᵣₐₙₛ), 5.26 (d, 1 H, J_{cis} 8.4 Hz, CH═CH_{cis}), 4.50 (d, 1 H, J_{1′,2′} 8.0 Hz, H-1′), 4.42 (d, 1 H, J_{1,2} 7.7 Hz, H-1), 4.37 (m, 1H, OCH), 4.20 (m, 1 H, OCH), 3.95 (dd, 1 H, J_{6a,6b} 12.2, J_{6a,5} 1.7 Hz, H-6a), 3.89 (d, 1 H, J_{4′,3′} 3.1 Hz, H-4′), 3.52 (dd, 1 H, J_{4,5} 9.8, J_{4,3} 7.8 Hz, H-4).

Anal. Calcd. for C₁₅H₂₆O₁₁.½H₂O: C, 46.03; H, 6.95. Found: C, 46.52; H, 6.85.

4-Pentenyl 2,2′,3,3′,4′,6,6′-hepta-Oacetyl-β-lactoside (6)

1 (0.5 g, 0.72 mmol) was stirred in anhydrous CH₂Cl₂ (5 mL) containing 4 Å sieves under an atmosphere of nitrogen when 4-penten-1-ol (0.37 mL, 3.60 mmol) and Hg(CN)₂ (0.3 g, 1.08 mmol) were added along with a crystal of iodine. The reaction mixture was covered and stirred at room temperature for 13.5 h before it was filtered through Celite®. The filtrate was evaporated in vacuo and the residue was chromatographed over silica gel (PE:EtOAc, 3:2 to 1:1) to yield 6 (0.21 g, 41%) as a colourless gum. ¹H NMR (CDCl₃, 400 MHz): δ 5.75 (m, 1 H, CH═CH,), 5.32 (dd, 1 H, J_{4′,3′} 3.4, J_{4′,5′} 0.8 Hz, H4′), 5.17 (dd, 1 H, J_{3,2} 9.4, J_{3,4} 9.2 Hz, H-3), 5.08 (dd, 1 H, J_{2′,3′} 10.4, J_{2′,1′} 7.9 Hz, H-2′), 4.98 (ddd, 1 H, Jₜᵣₐₙₛ 17.1, J 3.4, J_{gem} 1.7 Hz, CH═CHₜᵣₐₙₛ), 4.93 (dd, 1 H, J_{3′,2′} 10.4, J_{3′,4′} 3.4 Hz, H-3′), 4.92-4.96 (m, 1 H, CH═CH_{cis}), 4.87 (dd, 1 H, J_{2,3} 9.4, J_{2,1} 8.0 Hz, H-2), 4.46 (d, 1 H, J_{1′,2′} 7.9 Hz, H-1′), 4.43 (d, 1 H, J_{1,2} 8.0 Hz, H-1), 4.42-4.46 (m, 1 H, H-6a), 4.02-4.14 (m, 3 H, H-6b, H-6a′, H-6b′), 3.87 (dd, 1 H, J_{4,5} 9.7, J_{4,3} 9.2 Hz, H4), 3.84 (m, 1 H, H-5′), 3.81 (dt, 1 H, J 9.8, J 6.2 Hz, OCH), 3.57 (ddd, 1 H, J_{5,4} 9.7, J_{5,6b} 5.1, J_{5,6a} 2.0 Hz, H-5), 3.45 (dt, 1 H, J 9.8, J6.7 Hz, OCH), 2.14,2.12, 2.09,2.03, 2.02, 2.01, 1.94 (s, 21 H, 7×OAc), 1.52-1.70 (m, 4 H, CH₂CH₂).

4-Pentenyl β-lactoside (7)

To a solution of 6 (0.45 g, 0.63 mmol) in anhydrous MeOH (20 mL) under nitrogen was added a catalytic amount of sodium methoxide until the pH of the solution was around 10. The reaction mixture was then stirred overnight at room temperature before it was neutralized with acidic Amberlyte® resin. After the solvent was evaporated in vacuo, chromatography of the resulting residue over silica gel (EtOAc:MeOH:H₂O, 15:4:1) yielded 7 (0.17 g, 67%) as a white powder. ¹H NMR (D₂O, 400 MHz) selected data only: δ 5.89 (m, 1 H, CH═CH₂), 5.06 (dd, 1 H, Jₜᵣₐₙₛ17.3, J_{gem} 1.0 Hz, CH═CHₜᵣₐₙₛ), 5.00 (dd, 1 H, J_{cis} 9.3, J_{gem} 1.0 Hz, CH═CH_{cis}), 4.45 (d, 1 H, J_{1′,2′} 7.8 Hz, H-1′), 4.42 (d, 1 H, J_{1,2} 7.6 Hz, H-1), 2.07-2.17 (m, 2 H, CH₂CH═CH₂), 1.65-1.75 (m, 2 H, OCH₂—CH₂).

Anal. Calcd. for C₁₇H₃₀O₁₁.½H₂O: C, 48.68; H, 7.45. Found: C, 48.98; H, 7.14.

2,3-Dihydroxypropyl 2,2′,3,3′,4′,6,6′-hepta-O-acetyl-β-lactoside (8)

N-Methylmorpholino N-oxide (0.02 g, 0.18 mmol) was dissolved in a solution of 4:1 acetone:water (1.5 mL) under an atmosphere of nitrogen at 0° C. when a catalytic amount of osmium tetroxide in t-butanol was added. To this was added a solution of 4 (0.11 g, 0.16 mmol) in acetone (0.5 mL) and the reaction mixture was stirred overnight. Sodium bisulfite (0.06 g, 0.55 mmol) in water (1 mL) was then added to the reaction and stirring was continued for 1 h. The reaction was poured into brine (10 mL) and extracted with CH₂Cl₂ (2×10 mL). Evaporation of the combined organic layers after drying over MgSO₄ yielded 8 (0.11 g, 96%) as a white solid. ¹H NMR (CDCl₃, 400 MHz) selected data only: δ 5.35 (d, 1 H, J_{4′.,3′} 3.3 Hz, H-4′), 5.18 (dd, 1 H, J_{3,2} 9.5, J_{3,4} 9.1 HZ, H-3), 5.08 (dd, 1 H, J_{2′,3′} 10.4, J_{2′,1′} 7.9 Hz, H-2′), 4.94 (dd, 1 H, J_{3′,2′} 10.4, J_{3′,4′} 3.3 Hz, H-3′), 4.87 (dd, 1 H, J_{2,3} 9.5, J_{2,1} 8.0 Hz, H-2), 4.53 (ddd, 1 H, J 12.1, J 5.3, J 2.1 Hz, OCH), 4.44 (d, 1 H, J_{1′,2′} 7.9 Hz, H-1′), 4.42 (d, 1 H, J_{1,2} 8.0 Hz, H-1), 2.17, 2.13, 2.05, 2.03, 2.02, 1.94 (s, 21 H, 7×OAc).

2,3-Dihydroxypropyl β-lactoside (9)

To a solution of 8 (0.10 g, 0.15 mmol) in anhydrous MeOH (20 mL) under nitrogen was added a catalytic amount of sodium methoxide until the pH of the solution was around 10. The reaction mixture was then stirred overnight at room temperature before it was neutralized with acidic Amberlyte® resin. After evaporation of the solvent, crystalization of the resulting residue from MeOH yielded 9 (34.5 mg, 57%) as a white solid. ¹H NMR (D₂O, 400 MHz) selected data only: δ 4.47 (d, 1 H, J_{1,2} 8.0 Hz, H-1), 4.42 (d, 1 H, J_{1′,2′} 8.0 Hz, H-1′).

Anal. Calcd. for C₁₅H₂₈O₁₃: C, 43.27; H, 6.78. Found: C, 43.30; H, 6.92.

Synthesis of UDP-2FGal:

3,4,6-Tri-O-acetyl-2-deoxy-2-fluoro-D-galactopyranose (10)

3,4,6-Tri-O-acetyl-D-galactal (0.912 g, 3.349 mmol) was dissolved in DMF (15 mL), then water (6 mL) and Selectfluor™ (N-fluoro-N-chloromethyltriethylenediamine bis(tetrafluoroborate), 4.20 g, 11.80 mmol) from Air Products and Chemicals Inc. were added and the reaction mixture was stirred at 50° C. After 24 h, the reaction was shown to be complete by TLC (PE:EtOAc, 3:2). To the reaction mixture was added water (40 mL) and this was then extracted with CH₂Cl₂ (3×50 mL). The combined organic layers were washed with water (3×50 mL), dried over MgSO₄ and the solvent was removed under reduced pressure. Chromatography of the resulting residue over silica gel (PE:EtOAc, 3:2 to 1: 1) yielded 10 (0.36 g, 35%) as a colourless gum. ¹H NMR (CDCl₃, 400 MHz) for the α anomer: δ 5.52 (d, 1 H, J_{1,2} 3.8 Hz, H-1), 5.38-5.50 (m, 2 H, H-3, H-4), 4.75 (ddd, 1 H, J_{2,F} 49.9, J_{2,3} 10.0, J_{2,1} 3.8 Hz, H-2), 4.47 (m, 1 H, H-5), 4.00-4.15 (m, 2 H H-6a, H-6b), 2.10, 2.00, 1.99 (s, 9 H, 3×OAc). ¹⁹FNMR(CDCl₃, 188 MHz): δ-131.1 (dd, J_{F,2} 49.9, J_{F,3} 14.5 Hz).

1,3,4,6-Tetra-O-acetyl-2-deoxy-2-fluoro-β-galactopyranose (11)

To a solution of 10 (0.88 g, 2.84 mmol) in pyridine (7 mL) was added acetic anhydride (3.5 mL) and the reaction was stirred at room temperature overnight The pyridine and acetic anhydride were then removed by evaporation under reduced pressure and the residue was then taken up in 10% v/v HCl (80 mL) and extracted with CH₂Cl₂ (3×70 mL). The combined organic extracts were washed with 10% v/v HCl (70 mL), aq. NaHCO₃ (70 mL), water (70 mL) and brine (70 mL), dried over MgSO₄ and the solvent was evaporated in vacuo to yield the desired compound as a mixture of anomers. The residue was then dissolved in anhydrous CH₂Cl₂ (10 mL) under an atmosphere of argon and the temperature was brought to 0° C. To this was added a solution of 45% HBr/AcOH after which the argon source was removed and the reaction vessel was sealed and allowed to warm to room temperature. After 4 h, the reaction was poured into ice water (80 mL) and diluted with CH₂Cl₂ (80 mL). Solid NaHCO₃ was added to neutralize the excess acid and the layers were separated. The aqueous layer was further extracted with CH₂Cl₂ (2×70 mL). The organic layers were subsequently combined and washed with aq. NaHCO₃ (2×70 mL), water (100 mL) and brine (70 mL) and dried over MgSO₄. Evaporation of the solvent under reduced pressure yielded a beige gum to which acetic acid (35 mL) and Hg(OAc)₂ (1.87 g, 5.97 mmol) were added. The reaction was allowed to stir at room temperature under an atmosphere of argon. After 3 h, the reaction was poured into water (100 mL) and then extracted with CH₂Cl₂ (3×80 mL). The combined organic extracts were washed with aq. NaHCO₃ (3×80 mL), water (80 mL) and brine (80 mL). After drying over MgSO₄, the solvent was removed in vacuo and the residue was chromatographed over silica gel (PE:EtOAc, 3:1 to 5:2) to yield 11 (0.83 g, 83%) as a white solid. ¹H NMR (CDCl₃, 400 MHz): δ 5.77 (dd, 1 H, J_{1,2} 8.1, J_{1,F} 4.1 Hz, H-1), 5.43 (m, 1 H, H-4), 5.15 (ddd, 1 H, J_{3,F} 13.2, J_{3,2} 9.8, J_{3,4} 3.6 Hz, H-3), 4.62 (ddd, 1H, J_{2,F} 51.6, J_{2,3} 9.8, J_{2,1} 8.1 Hz, H-2), 4.05-4.20 (m, 3 H, H-5, H-6a, H6b), 2.18, 2.14, 2.05, 2.03 (s, 12 H, 4×OAc). ¹⁹F NMR (CDCl₃, 188 MHz): δ-132.1 (ddd, J_{F,2} 51.6, J_{F,3} 13.2, J_{F,1} 4.1 Hz).

2-Deoxy-2-fluoro-α-D-galactopyranose-1-phosphate, mono(tri-n-octyl)ammonium Salt (12)

Anhydrous H₃PO₄ (0.31 g, 3.17 mmol) was dried under vacuum for 24 h before it was melted at 50° C. 11 (0.14 g, 0.40 mmol) was then added and the reaction mixture was stirred under reduced pressure (20 mm Hg) for 9 h. After this time, THF (1 mL) and a solution of 2 M LiOH (6 mL) were added and the reaction mixture was allowed to stir at room temperature overnight After filtering through Celite® and washing with 0.01 M LiOH, the solvent was evaporated in vacuo. The residue was then dissolved in water and passed through a column of Bio-Rad AG 50W-X2, 200-400 mesh, sulfonic acid cation exchanger (H⁺ form). The desired fractions were pooled and the solvent volume was decreased by evaporation under reduced pressure. Tri-n-octylamine (0.14 g, 0.40 mmol) was added and the solution was lyophilized yielding 12 (0.28 g) as a colourless syrup. ¹H NMR (CDCl₃, 400 MHz): δ 5.74 (dd, 1 H, J_{1,P} 5-9, J_{1,2} 3.6 Hz, H-1), 4.63 (m, 1 H, H-2), 4.23 (m, 1 H, H-3), 3.72-4.15 (m, 4 H, H-4, H-5, H-6a, H-6b), 2.80 (m, 6 H, NCH₂), 1.65 (m, 6 H, NCCH₂), 1.30 (m, 30 H, CH₂), 0.85 (t, 9 H, CH₃). ¹⁹F NMR (CDCl₃, 188 MHz, proton decoupled): δ-132.4. ³¹P NMR (CDCl₃, 81 MHz proton decoupled): δ 0.01.

Uridine 5′-diphospho-(2-deoxy-2-fluoro)-α-D-galactopyranose, di-ammonium Salt (13)

To 12 (0.25 g, 0.40 mmol) was added anhydrous pyridine (5 mL) which was then evaporated. This procedure was repeated twice before UMP-morpholidate (0.33 g, 0.48 mmol) was added. Evaporation with anhydrous pyridine (5 mL) was again repeated three times. 1H tetrazole (0.07 g, 1.01 mmol) and anhydrous pyridine (3 mL) were then added and the reaction mixture was stirred at room temperature. An aliquot of the reaction mixture was transferred into an NMR tube containing a capillary of DMSO-d6 so that the progress of the reaction could be monitored via ³¹P NMR. After 27 days, the reaction mixture was diluted with water and evaporated under reduced pressure. After repeating this four times, the residue was taken up in 100 mM NH₄HCO₃ (5 mL) and the tri-n-octylamine was extracted with diethyl ether (3×5 mL). The aqueous layer was lyophilized to yield the crude product Purification was afforded by size exclusion chromatography through a column of Bio-Gel P2 extra fine resin (1×45 cm) using a Beckman Biosepra ProSys Workstation. The product was eluted with 250 mM NH₄HCO₃ at a flow rate of 0.1 mL/min. The desired fractions were pooled and lyophilized to yield 13 (90.0 mg, 37%) as a white powdery solid. ¹H NMR (D₂O, 400 MHz) selected data only: δ 7.91 (d, 1 H, J_{6,5} 6.1 HZ H-6), 5.93 (m, 2 H, H-1′, H5), 5.76 (dd, 1 H J_{1″,P} 7.1, J_{1″,2} 3.6, H-1″). ¹⁹F NMR (D₂O, 188 MHz): δ-132.4 (dd, J_{2″,F} 49.9, J^{3″,F} 11.1 Hz). ³¹P NMR (D₂O, 81 MHz, proton decoupled): δ-9.10 (d, JPβ,Pα 19.9 Hz, P^{β}), −10.8 (d, J_{Pα,Pβ} 19.9 Hz, P^{α}).

Anal. Calcd. for C₁₅H₂₉FN₄O₁₆P₂: C, 29.91; H, 4.85; N, 9.30. Found: C, 30.37; H, 5.34; N, 9.89.

Synthesis of Incompetent Acceptor Substrates:

1,2,2′,3,3′,6-Hexa-O-acelyl-4′,6′-O-benzylidene-α-lactose (14)

To a suspension of lactose (45 g, 124.90 mmol) in DMF (110 mL) was added benzaldehyde dimethyl acetal (20.6 mL, 137.40 mmol) followed by a catalytic amount of p-toluenesulphonic acid (0.47 g, 2.5 mmol). The reaction mixture was then stirred under reduced pressure (20 mm Hg) for 4 d at 60° C. After this time, water (200 mL) was added and unreacted benzaldehyde dimethyl acetal was extracted with EtOAc (2×200 mL). The aqueous layer was evaporated in vacuo following which, pyridine (200 mL) and acetic anhydride (100 mL) were then added to the resulting residue. The reaction mixture was allowed to stir overnight before the volume was decreased by evaporation under reduced pressure. To the remaining residue was added ice water (300 mL) and the crude product was then extracted with CH₂Cl₂ (2×250 mL). The combined organic layers were washed with 10% v/v HCl (3×200 mL), water (2×200 mL) and brine (1×250 mL), dried over MgSO₄ and the solvent was evaporated under reduced pressure. Crystallization from EtOAc/Hexane yielded 14 as a slightly yellowish solid (8 g, 10%). ¹H NMR (CDCl₃, 200 MHz): δ 7.30-7.55 (m, 5 H, Ar), 6.45 (d, 1 H, J_{1,2} 3.7 Hz, H-1), 5.48 (s, 1 H, PhCH), 5.45 (dd, 1 H, J_{3,2} 10.4, J_{3,4} 9.9 Hz, H-3), 5.35 (dd, 1 H, J_{2′,3′} 10.3, J_{2′,1′} 7.9 Hz, H-2′), 5.05 (dd, 1 H, J_{2,3} 10.4, J_{2,1} 3.7 Hz, H-2), 4.87 (dd, 1 H, J_{3′,2′} 10.3, J_{3′,4′} 3.5 Hz, H-3′), 4.45 (d, 1 H, J_{1′,2′} 7.9 Hz, H-1′), 4.30-4.55 (m, 1 H, H-5), 4.15-4.45 (m, 2 H, H6a, H-6b), 4.13 (dd, 1 H, J_{6a′,6b′} 8.2, J_{6a′,5′} 4.1 Hz, H-6a′), 4.08 (dd, 1 H, J_{4′,3′} 3.5, J_{4′,5′} 1.6 Hz, H-4), 3.95-4.05 (m, 2 H, H-5′, H-6b′), 3.80 (dd, 1 H, J_{4,3} 9.9, J_{4,5} 9.3 Hz, H-4), 2.17, 2.11, 2.15, 2.14, 2.00 (s, 18 H, 6×OAc).

1,2,2′,3,3′,6-Hexa-O-acetyl-6′-O-benzyl-α-lactose (15)

14 (3.5 g, 5.13 mmol) was dissolved in anhydrous THF (120 mL) when sodium cyanoborohydride (3.2 g, 51.27 mmol) was added. A saturated solution of HCl in diethyl ether was then cannulated into the reaction mixture in portions until the evolution of gas had ceased. Within 0.5 h, the reaction was judged to be complete by TLC (PE:EtOAc, 1:1). At this time, the reaction mixture was added to water (100 mL) and the crude product was extracted with CH₂Cl₂ (2×100 mL). The combined organic layers were washed with aq NaHCO₃ (2×100 mL) and water (100 mL), dried over MgSO₄ and evaporated in vacuo. Chromatography over silica gel (PE:EtOAc, 4:5) yielded 15 as a white solid (2.66 g, 76%). ¹H NMR (CDCl₃, 200 MHz) selected data only: δ 7.30-7.45 (m, 5 H, Ar), 6.25 (d, 1 H, J_{1,2} 3.7 Hz, H-1), 5.43 (dd, 1 H, J_{3,2} 10.2, J_{3,4} 9.1 Hz, H-3), 5.19 (dd, 1 H, J_{2′,3′} 10.1, J_{2′,1′} 7.8 Hz, H-2′), 5.02 (dd, 1 H, J_{2,3} 10.2, J_{2,1} 3.7 Hz, H-2), 4.89 (dd, 1H, J_{3′,2′} 10.1, J_{3′,4′} 3.1 Hz, H-3′), 4.52 (s, 2 H, PhCH₂), 4.45 (d, 1 H, J_{1′,2′} 7.8 Hz, H-1′), 2.15, 2.10, 2.07, 2.04, 2.01,2.00 (s, 18 H, 6×OAc).

1,2,2′,3,3′,6-Hexa-O-acetyl-6′-O-benzyl-4′-deoxy-4′-iodo-α-cellobiose (16)

A stirring solution of 15 (1.32 g, 1.93 mmol) in anhydrous CH₂Cl₂ (15 mL) under argon was cooled to −20° C. before pyridine (5 mL) and triflic anhydride (0.88 mL, 5.20 mmol) were added The reaction mixture was then warmed to room temperature and stirred for 2 h prior to the addition of aq NaHCO₃ (50 mL). The crude material was extracted with CH₂Cl₂ (2×35 mL) and the combined organic extracts were subsequently washed with water (2×80 mL). Coevaporation of the organic layer with MeCN under reduced pressure yielded a yellow foamy gum, which was then dissolved in anhydrous DMF (25 mL). After the addition of Nal (1.44 g, 9.63 mmol), the reaction mixture was allowed to stir at room temperature overnight under an atmosphere of argon before being added to aq NaHCO₃ (100 mL) and extracted with CH₂Cl₂ (3×125 mL). After the combined organic layers were washed with water (2×75 mL), dried over MgSO₄ and evaporated under reduced pressure, the resulting residue was crystallized from EtOAc/Hexane to yield 16 (0.94 g, 62%) as a cotton-like solid. ¹H NMR (CDCl₃, 200 MHz) selected data only: δ 7.30-7.45 (m, 5 H, Ar), 6.24 (d, 1 H, J_{1,2} 3.7 Hz, H-1), 5.42 (dd, 1 H, J_{3,2} 10.3, J_{3,4} 9.3 Hz, H-3), 5.25 (dd, 1 H, J_{3′,2′} 9.2, J_{3′,4′} 11.0 Hz, H-3′), 5.00 (dd, 1 H, J₂₃ 10.3, J_{2,1} 3.7 Hz, H-2), 4.78 (dd, 1 H, J_{2′,3′} 9.2, J_{2′,1′} 8.1 Hz, H-2′), 4.50 (s, 2 H, PhCH₂), 4.48 (d, 1 H, J_{1′,2′} 8.1 Hz, H-1′), 2.12,2.09,2.06,2.01, 2.00 (s, 18 H, 6×OAc).

1,2,2′,3,3′,6-Hexa-O-acetyl-6′-O-benzyl-4′-deoxy-α-lactose (17)

To a solution of 16 (0.90 g, 1.13 mmol) in anhydrous benzene (30 mL) under argon was added tributyltin hydride (1.65 g, 5.67 mmol) and a catalytic amount of AIBN. The reaction mixture was then refluxed for 45 min after which time the solvent was evaporated in vacuo. The residue was dissolved in MeCN (120 mL) and washed with hexane (3×70 mL). Evaporation of the MeCN layer followed by crystallization from EtOAc/Hexane yielded 17 (0.87 g, 85%) as a white fluffy solid. ¹H NMR (CDCl₃, 400 MHz): δ 7.25-7.40 (m, S H, Ar), 6.24 (d, 1 H, J_{1,2} 3.7 Hz, H-1), 5.42 (dd, 1 H, J_{3,2} 10.1, J_{3,4} 9.4 H, H-3), 4.99 (dd, 1 H, J₂₃ 10.1, J_{2,1} 3.7 HZ, H-2), 4.91 (ddd, 1 H, J_{3′,4′ax} 11.3, J_{3′,2′} 9.6, J_{3′,4′eq} 5.4 Hz, H-3′), 4.80 (dd, 1 H, J_{2′,3′} 9.6, J_{2′,1′} 7.8 Hz, H-1′), 4.50 (s, 2 H, PhCH₂), 4.43 (dd, 1 H, J_{6a,6b} 12.2, J_{6a,5} 2.0 Hz, H-6a), 4.36 (d, 1 H, J_{1′,2+} 7.8 Hz, H-1′), 4.12 (dd, 1 H, J_{6b,6a} 12.2, J_{6b,5} 4.3 Hz, H-6b), 3.97 (ddd, 1 H, J_{5,4} 10.1, J_{5,6b} 4.3, J_{5,6a} 2.0 Hz, H-5), 3.78 (dd, 1 H, J_{4,5} 10.1, J_{4,3} 9.4 Hz, H-4), 3.60-3.67 (m, 1 H, H-5′), 3.56 (dd, 1 H, J_{6a′,6b′} 9.8, J_{6a′,5′} 5.3 Hz, H-6a′), 3.45 (dd, 1 H, J_{6b′,6a′} 9.8, J_{6b′,5′} 4.8 Hz, H-6b′), 2.14, 2.08, 2.01, 1.98 (s, 18 H, 6×OAc), 2.00-2.10 (m, 1 H, H4′_{eq}), 1.50-1.64 (m, 1 H, H-4′ₐₓ).

1,2,2′,3,3′,6-Hexa-O-acetyl-4′-deoxy-α-lactose (18)

17 (0.23 g, 0.34 mmol) was dissolved in EtOH (4 mL) when cyclohexene (1.39 mL, 13.76 mmol) and 20% Pd(OH)₂/C (0.06 g) were added and the reaction mixture was refluxed. After 2 h, the catalyst was removed by filtration through Celite® and the filtrate was evaporated in vacuo. Crystallization from EtOH yielded 18 (0.13 g, 63%) as a white fluffy solid. ¹H NMR (CDCl₃, 400 MHz): δ 6.24 (d, 1 H, J_{1,2} 3.7 Hz, H-1), 5.45 (dd, 1 H, J_{3,2} 10.0, J_{3,4} 9.2 Hz, H-3), 5.01 (dd, 1 H, J_{2,3} 10.0, J_{2,1} 3.8 Hz, H-2), 4.94 (ddd, 1 H, J_{3′,4′ax} 11.5, J_{3′,2′} 9.5, J_{3′,4′eq} 5.4 Hz, H-3′), 4.81 (dd, 1 H, J_{2′,3′} 9.5, J_{2′,1′} 7.7 Hz, H-2′), 4.47 (d, 1 H, J_{1′,2′} 7.7 Hz, H-1′), 4.44 (dd, 1 H, J_{6a,6b} 12.5, J_{6a,5} 2.0 Hz, H-6a), 4.08 (dd, 1 H, J_{6b,6a} 12.5, J_{6b,5} 4.5 Hz, H-6b), 4.00 (ddd, 1 H, J_{5,4} 10.0, J_{5,6b} 4.5, J_{5,6a} 2.0 Hz, H-5), 3.83 (dd, 1 H, J_{4,5} 10.0, J_{4,3} 9.2 Hz, H-4), 3.52-3.68 (m, 3 H, H-5′, H-6a′, H-6b′), 2.15, 2.08, 2.06, 2.04, 1.98, 1.97 (s, 18 H, 6×OAc), 2.00-2.10 (m, 1 H, H-4′_{eq}), 1.50-1.64 (m, 1 H, H-4′ₐₓ).

4′-Deoxylactose (19)

To a stirring solution of 18 (0.50 g, 0.87 mmol) in anhydrous MeOH (8 mL) under argon was added a catalytic amount of sodium methoxide until the solution was slightly basic. The reaction mixture was allowed to stir at room temperature overnight prior to being neutralized with Amberlyte® IR-120 acidic resin. After filtration, evaporation of the solvent under reduced pressure yielded 19 (0.24 g, 85%) as a white solid. ¹H NMR (D₂O, 400 MHz) selected data only for β anomer: δ 4.63 (d, 1 H, J_{1,2} 8.0 Hz, H-1), 4.40 (d, 1 H, J_{1′,2′} 7.9 Hz, H-1′), 3.22-3.29 (m, 1 H, H-5), 3.19 (ddd, 1 H, J_{5′,4′ax} 11.2, J_{5′,6a′} 8.0, J_{5′,6b′} 3.2 Hz, H-5′), 1.95 (dd, 1 H, J_{4′eq,4′ax} 12.0, J_{4′eq,3′} 4.4 Hz, H-4′_{eq}), 1.42 (ddd, 1 H, J_{4′ax,4′eq} 12.0, J_{4′ax,3′} 11.9, J_{4′ax,5′} 11.2 Hz, H-4′ₐₓ).

Anal. Calcd. for C₁₂H₂₂O₁₀: C, 44.17; H, 6.80. Found: C, 43.87; H, 6.91.

2,2′,3,3′,4′,6,6′-Hepta-O-acetyl-α-cellobiosyl Bromide (20)

A solution of octa-O-acetylcellobiose (10.95 g, 16.10 mmol) in anhydrous CH₂Cl₂ (35 mL) under an atmosphere of nitrogen was cooled to 0° C. prior to the addition of 45% w/v HBr/AcOH (13 mL). The reaction vessel was then sealed and the reaction mixture was allowed to warm to room temperature. After 4 h, the reaction mixture was diluted with CH₂Cl₂ (100 mL) and the solution was added to ice water (200 mL) whereupon it was neutralized with solid NaHCO₃. Upon separation of the two phases, the aqueous layer was further extracted with CH₂Cl₂ (2×100 mL) and the combined organic extracts were washed with aq. NaHCO₃ (2×75 mL), water (75 mL) and brine (75 mL). Removal of the solvent under reduced pressure after drying over MgSO₄ yielded 20 as a white solid (11.12 g, 99%). ¹H NMR (CDCl₃, 400 MH): δ 6.50 (d, 1 H, J_{1,2} 4.1 Hz, H-1), 5.50 (dd, 1 H, J_{3,2} 9.7, J_{3,4} 9.7 Hz, H-3), 5.13 (dd, 1 H, J_{3′,4′} 9.3, J_{3′,2′} 9.2 Hz, H-3′), 5.05 (dd, 1 H, J_{4′,5′} 9.7, J_{4′,3′} 9.3 Hz, H-4′), 4.91 (dd, 1 H, J_{6a′,6b′} 12.5, J_{6a′,5′} 4.5 Hz, H-6a′), 4.11-4.23 (m, 2 H, H-5, H-6b), 4.03 (dd, 1 H, J_{6b′,6a′} 12.5, J_{6b′,5′} 2.3 Hz, H-6b′), 3.81 (dd, 1 H, J_{4,3} 9.7, J_{4,5} 9.7 Hz, H-4), 3.65 (ddd, 1 H, J_{5′,4′} 9.7, J_{5′,6a′} 4.5, J_{5′,6b′} 2.3 Hz, H-5′), 2.11, 2.06, 2.01, 2.00, 1.98, 1.96 (s,21 H, 7×OAc).

Benzyl 2,2′,3,3′,4′,6,6′-hepta-O-acetyl-β-cellobioside (21)

To a solution of 20 (9.99 g, 14.29 mmol) in anhydrous CH₂Cl₂ (100 mL) containing 4 Å molecular sieves under an atmosphere of nitrogen was added benzyl alcohol (7.39 mL, 71.44 mmol) and silver carbonate (7.88 g, 28.58 mmol). A crystal of iodine was added and the reaction vessel was shielded from the light and stirred at room temperature overnight After this time, the reaction mixture was filtered through Celite® and washed with CH₂Cl₂. The solvent was then evaporated in vacuo and the residue was crystallized from EtOAc/Hex to yield 21 (6.91 g, 66%) as a white fluffy solid. ¹H NMR (CDCl₃, 400 MHz): δ 7.20-7.35 (m, 5 H, Ar), 5.11 (dd, 2 H, J_{3,2/3′,2′} 9.3, J_{3,4/3′,4′} 9.3 Hz, H-3, H-3′), 5.03 (dd, 1 H, J_{4′,5′} 9.8, J_{4′,3′} 9.3 Hz, H-4′), 4.94 (dd, 1 H, J_{2′,3′} 9.6, J_{2′,1′} 7.9 Hz, H-2′), 4.89 (dd, 1 H, J_{2,3} 9.3, J_{2,1} 8.0 Hz, H-2), 4.83 (d, 1 H, J 12.3 Hz, PhCH). 4.56 (d, 1 H, J 12.3 Hz, PhCH), 4.51 (dd, 1 H, J_{6a,6b} 12.0, J_{6a,5} 2.0 Hz, H-6a), 4.49 (d, 1 H, J_{1,2} 8.0 Hz, H-1), 4.48 (d, 1 H, J_{1′,2′} 7.9 Hz, H-1′), 4.33 (dd, 1 H, J_{6a′,6b′} 12.5, J_{6a′,5′} 4.5 Hz, H-6a′), 4.08 (dd, 1 H, J_{6b,6a} 12.0, J_{6b,5} 5.0 Hz, H-6b), 4.01 (dd, 1 H, J_{6b′,6a′} 12.5, J_{6b′,5′} 2.3 Hz, H-6b′), 3.77 (dd, 1 H, J_{4,5} 9.6, J_{4,3} 9.3 Hz, H-4), 3.63 (ddd, 1 H, J_{5′,4′} 9.8, J_{5′,6b′} 4.5, J_{5′,6b′} 2.3 Hz, H-S′), 3.54 (ddd, 1 H, J_{5,4} 9.6, J_{5,6b} 5.0, J_{5,6a} 2.0 Hz, H-5), 2.12, 2.05, 2.00, 1.98, 1.97, 1.95 (s, 21 H, 7×OAc).

Benzyl β-cellobioside (22)

21 (6.33 g, 8.71 mmol) was suspended in anhydrous MeOH under nitrogen when a catalytic amount of sodium methoxide was added until a basic pH was obtained. After 24 h, the reaction mixture was neutralized with acidic Amberlyte® resin. Removal of the solvent under reduced pressure yielded 22 (3.91, g) quantitatively as a white solid. ¹H NMR (D₂O, 400 MHz) selected data only: δ 7.35-7.50 (m, 5 H, Ar), 4.90 (d, 1H, J 11.6 Hz, PhCH), 4.75 (d, 1 H, J 11.6 Hz, PhCH), 4.56 (d, 1 H, J_{1,2} 8.0 Hz, H-1), 4.47 (d, 1 H, J_{1′,2′} 7.8 Hz, H-1′), 3.96 (dd, 1 H, J_{6a,6b} 12.1, J_{6a,5} 2.0 Hz, H-6a), 3.88 (dd, 1 H, J_{6a′,6b′} 12.6, J_{6′,5′} 2.2 Hz, H-6a′), 3.79 (dd, 1 H, J_{6b,6a} 12.1, J_{6b,5} 5.0 Hz H-6b), 3.70 (dd, 1 H, J^{6b′,6a′} 12.6, J_{6′,5′} 5.6 Hz, H-6b′).

Benzyl 2,2′,3,3′,6-penta-O-acetyl-4′,6′-O-p-methoxybenzylidene-β-cellobioside (23)

To a suspension of 22 (3.64 g, 8.42 mmol) in DMF (100 mL) was added p-anisaldehyde dimethyl acetal (1.72 mL, 10.10 mmol) followed by a catalytic amount of toluenesulphonic acid (0.03 g, 0.17 mmol). The reaction mixture was then stirred under reduced pressure (20 mm Hg) for 10 d at 60° C. After this time, solid NaHCO₃ was added to neutralize the reaction and the solvent was evaporated in vacuo. To the resulting residue was added pyridine (30 mL) and acetic anhydride (15 mL) and the mixture was stirred overnight. The volume of pyridine and acetic anhydride was then decreased by evaporation under reduced pressure and the resulting syrup was taken up in 10% v/v HCl and extracted with CH₂Cl₂ (3×80 mL). The combined organic extracts were washed with 10% v/v HCl (2×50 mL), aq. NaHCO₃ (50 mL) and water (50 mL), dried over MgSO₄ and the solvent was evaporated in vacuo. The residue was crystallized from EtOAc/Hexanes to yield 23 (3.72 g, 58%) as a white solid. ¹H NMR (CDCl₃, 400 MHz): δ 7.20-7.35 (m, 7 H. Ar), 6.80-6.90 (m, 2 H, Ar), 5.40 (s, 1 H, MeOPhCH), 5.22 (dd, 1 H, J_{3,2} 9.4, J_{3,4} 9.3 Hz, H-3), 5.11 (dd, 1 H, J_{3′,4′} 9.3, J_{3′,2′} 9.2 Hz, H-3′), 4.94 (dd, 1 H, J_{2,3} 7.9 Hz, H-2), 4.88 (dd, 1 H, J_{2′,3′} 9.2, J_{2′,1′} 7.8 Hz, H-2′), 4.84 (d, 1 H, J 12.3 Hz, PhCH), 4.57 (d, 1 H, J_{1,2} 7.9 Hz, H-1), 4.56 (d, 1 H, J 12.3 Hz, PhCH), 4.50 (dd, 1 H, J_{6a,6b} 11.9, J_{6a,5} 1.9 Hz, H-6a), 4.49 (d, 1 H, J_{1′,2′} 7.8 Hz, H-1′), 4.31 (dd, 1 H, J_{4′,3′} 9.3, J_{4′,5′} 4.9 Hz, H-4′), 4.07 (dd, 1 H, J_{6b,6a} 11.9, J_{6b,6a} 4.7 Hz, H-6b), 3.75-3.90 (m, 4 H, H-4, OCH₃), 3.66 (dd, 1 H, J_{6a′,6b′} 10.2, J_{6a′,5′} 9.6 Hz, H-6a′), 3.64 (dd, 1 H, J_{6b′,6a′} 10.2, J_{6b′,5′} 9.3 Hz, H-6b′), 3.53 (ddd, 1 H, J_{5,4} 9.8, J_{5,6b} 4.7, J_{5,6a} 1.9 Hz, H-5), 3.43 (ddd, 1 H, J_{5′,6a′} 9.6, J_{5′,6b′} 9.3, J_{5′,4′}4.9 Hz, H-5′), 2.12, 2.03, 2.00, 1.99, 1.97 (s, 15 H, 5×OAc).

Benzyl 2,2′,3,3′,6-penta-O-acetyl-6′-O-p-methoxybenzyl-β-cellobioside (24)

23 (1.17 g, 1.54 mmol) was dissolved in anhydrous DMF (12 mL) when NaCNBH₃ (0.49 g, 7.72 mmol) was added. A solution of TFA (1.20 mL, 15.43 mmol) in anhydrous DMF (8 mL) was then added dropwise to the reaction mixture and the solution was allowed to stir at room temperature overnight. The reaction mixture was then filtered through Celite® and washed with CH₂Cl₂. The filtrate was added to aq NaHCO₃ (100 mL) and extracted with additional portions of CH₂Cl₂ (3×75 mL). The combined organic extracts were then washed with aq NaHCO₃ (100 mL), water (2×100 mL) and brine (100 mL), dried over MgSO₄ and the solvent was removed under reduced pressure. Chromatography over silica gel (PE:EtOAc, 1:1) afforded 24 (0.85 g, 72%) as a white solid. ¹H NMR (CDCl₃, 400 MHz): δ 7.15-7.35 (m, 7 H, Ar), 6.80-6.90 (m, 2 H, Ar), 5.09 (dd, 1 H, J_{3,2} 9.3, J_{3,4} 9.2 Hz, H-3), 4.95 (dd, 1 H, J_{3′,2′} 9.4, J_{3′,4′} 9.3 Hz, H-3′), 4.93 (dd, 1 H, J_{2,3} 9.3, J_{2,1} 7.9 Hz, H-2), 4.82 (d, 1 H, J 12.3 Hz, PhCH), 4.80 (dd, 1 H, J_{2′,3′} 9.4, J_{2′,1′} 7.9 Hz, H-2′), 4.55 (d, 1 H, J 12.3 Hz, PhCH), 4.50 (dd, 1 H, J_{6a,6b} 12.0, J_{6a,5} 2.0 Hz, H-6a), 4.47 (d, 1 H, J 11.2 Hz, MeOPhCH), 4.46 (d, 1 H, J_{1,2} 7.9 Hz, H-1), 4.44 (d, 1 H, J_{1′,2′} 7.9 Hz, H-1′), 4.41 (d, 1 H, J 11.2 Hz, MeOPhCH), 4.06 (dd, 1 H, J_{6b,6a} 12.0, J_{6b,5} 5.0 Hz, H-6b), 3.67-3.76 (m, 3 H, H-4, H-4′, H-6a′), 3.64 (dd, 1 H, J_{6b′,6a′} 9.9, J_{6b′,5′} 4.8 Hz, H-6b′), 3.52 (ddd, 1 H, J_{5,4} 9.8, J_{5,6b} 5.0, J_{5,6a} 2.0 Hz, H-5), 3.39-3.45 (m, 1 H, H-5′), 2.10, 2.03, 2.00, 1.97, 1.94 (s, 15 H, 5×OAc).

Benzyl 2,2′,3,3′,6-penta-O-acetyl-4′-deoxy-4′-fluoro-β-lactoside (25)

A solution of 24 (0.11 g, 0.15 mmol) in anhydrous CH₂Cl₂ (5 mL) under an atmosphere of argon was cooled to −20° C. before trilic anhydride (66.4 μL, 0.40 mmol) was added dropwise to the solution. The reaction mixture was stirred at room temperature for 1 h and was then diluted with 10% v/v HCl (20 mL). The mixture was extracted with CH₂Cl₂ (3×20 mL) and the combined organic layers were washed with an additional portion of 10% v/v HCl (20 mL), water (2×20 mL) and brine. After drying over MgSO₄, the solvent was evaporated under reduced pressure. The resulting residue was then dissolved in anhydrous CH₂Cl₂ and the solution was cooled to −10° C. prior to the addition of tris(dimethylamino)sulfur (trimethylsilyl)difluoride (0.12 g, 0.44 mmol). The reaction was then refluxed for 0.5 h after which time water (20 mL) was added and the reaction was extracted with CH₂Cl₂ (3×20 mL). The combined organic extracts were washed with water (2×20 mL), dried over MgSO₄ and the solvent was removed in vacua. Chromatography over silica gel (PE:EtOAc, 2:3) yielded 25 (40.3 mg, 43%) as a white solid. ¹H NMR (CDCl₃, 500 MHz): δ 7.25-7.35 (m, 5 H, Ar), 5.15 (dd, 1 H, J_{2′,3′} 10.3, J_{2′,1′} 8.0 Hz, H-2′), 5.12 (dd, 1 H, J_{3,2} 9.2, J_{3,4} 9.2 Hz, H-3), 4.96 (dd, 1 H, J_{2,s} 9.2, J_{2,1} 7.8 Hz, H-2), 4.89 (ddd, 1 H, J_{3′,F} 27.6, J_{3′,2′} 10.4, J_{3′,4′} 2.7 Hz, H-3′), 4.84 (d, 1 H, J 12.1 Hz, PhCH), 4.80 (dd, 1 H, J_{4′,F} 50.3, J_{4′,3′} 2.7 Hz, H4′), 4.57 (d, 1 H, J12.3 Hz, PhCH), 4.52 (d, 1H, J_{1′,2′} 7.4 Hz, H-1′), 4.51 (dd, 1 H, J_{6a,6b} 11.9, J_{6a,5} 2.2 Hz, H-6a), 4.49 (d, 1 H, J_{1,2} 7.8 Hz, H-1), 4.07 (dd, 1 H, J_{6b,6a} 11.9, J_{6b,5} 5.2 Hz, H-6b), 3.87 (ddd, 1 H, J_{6a′,6b′} 11.5, J_{6a′,5′} 7.5, J_{6a′,F} 1.0 Hz, H-6a′), 3.85 (dd, 1 H, J_{4,3} 9.2, J_{4,5} 9.3 Hz, H4), 3.72 (dd, 1 H, J_{6b′,6a′} 11.5, J_{6b′,5′} 4.9 Hz, H-6b′), 3.61 (ddd, 1 H, J_{5′,F} 26.4, J_{5,6a′}7.5, J_{5′,6b′} 4.9 Hz, H-5′),. 3.55-3.59 (m, 1 H, H-5), 2.11, 2.06, 2.04, 2.03, 1.98 (s, 15 H, 5×OAc). ¹⁹F NMR (CDCl₃, 188 MHz): δ-140.0 (ddd, J_{F,4′} 50.3, J_{F,3′} 27.6, J_{F,5′} 26.4 Hz).

Anal. Calcd. for C29H₃₇FO: C, 54.04; H, 5.79. Found: C, 54.32; H,5.78.

Benzyl 4′-deoxy-4′-fluoro-β-lactoside (26)

A solution of 25 (0.09 g, 0.14 mmol) in anhydrous MeOH (5 mL) under argon was made basic through the addition of a catalytic amount of sodium methoxide. The reaction mixture was stirred at room temperature overnight before it was neutralized with acidic Amberlyte® resin. Removal of the resin by filtration followed by evaporation of the solvent in vacuo gave a quantitative yield of 26 (60 mg) as a white solid. ¹H NMR (D₂O, 400 MHz) selected data only: δ 7.35-7.55 (m, 5 H, Ar), 4.91 (d, 1 H, J 11.6 Hz, PhCH), 4.54 (d, 1 H, J_{1′,2′} 7.8 Hz, H-1′), 4.51 (d, 1 H, J_{1,2} 6.0 Hz, H-1), 3.98 (dd, 1 H, J_{6a,6b} 12.3, J_{6a,5} 1.9 Hz, H-6a), 3.33 (dd, 1 H, J_{4,3} 8.6, J_{4,5} 8.4 Hz, H4). ¹⁹F NMR (D₂O, 188 MHz): δ-141.2 (ddd, J_{F,4′} 51.0, J_{F,3′} 30.0, J_{F,5′} 30.0 Hz).

Anal. Calcd. for C₁₉H₂₇FO₁₀: C, 52.53; H, 6.26. Found: C, 52.33; H, 6.31.

Benzyl 2,2′,3,3′,6-penta-O-acetyl-4′-deoxy-4′-iodo-6′-O-p-methoxybenzyl-β-lactoside (27)

To a stirred solution of 24 (1.22 g, 1.60 mmol) in anhydrous CH₂Cl₂ (20 mL) under argon at −20° C. was added pyridine (1.1 mL, 13.47 mmol) followed by triflic anhydride (0.73 mL, 4.33 mmol). After 5 min, the reaction mixture was allowed to warm to room temperature and then was stirred at this temperature for an additional hour. After dilution with 10% v/v HCl (65 mL), the reaction was then extracted with CH₂Cl₂ (3×65 mL). The combined organic extracts were washed with 10% v/v HCl (65 mL), water (65 mL) and brine (65 mL), dried over MgSO₄ and the solvent was evaporated under reduced pressure. The residue was dissolved in anhydrous DMF (20 mL), Nal (1.20 g, 8.02 mmol) was added and the reaction mixture was stirred at room temperature overnight. After diluting with water (75 mL) and extracting with CH₂Cl₂ (3×75 mL), the combined organic layers were then washed with water (4×75 mL) and brine (75 mL), dried over MgSO₄ and the solvent was evaporated in vacuo. The residue was chromatographed over silica gel (PE:EtOAc, 3:2) and the desired fractions were pooled and recrystallized from EtOAc/Hex to yield 27 (0.54 g, 38%) as a white solid. ¹H NMR (CDCl₃, 400 MHz): δ 7.20-7.35 (m, 7 H, Ar), 6.85-6.90 (m, 2 H, Ar), 5.15 (dd, 1 H, J_{2′,3′} 9.9, J_{2′,1′} 7.8 Hz, H-2′), 5.10 (dd, 1 H, J_{3,2} 9.5, J_{3,4} 9.3 Hz, H-3), 4.95 (dd, 1 H, J_{2,3} 9.5, J_{2,1} 7.9 Hz, H-2), 4.83 (d, 1 H, J 12.3 Hz, PhCH), 4.64 (dd, 1 H, J_{4′,3′} 4.2, J_{4′,5′} 1.1 Hz, H-4′), 4.56 (d, 1 H, J 12.3 Hz, PhCH), 4.47 (d, 1 H, J_{1′,2′} 7.8 Hz, H-1′), 4.43-4.49 (m, 2 H, MeOPhCH₂), 4.43 (d, 1 H, J_{1,2} 7.9 Hz, H-1), 4.43 (dd, 1 H, J_{6a,6b} 11.9, J_{6a,5} 2.0 Hz, H-6a), 4.25 (dd, 1 H, J_{3′,2′} 9.9, J_{3′,4′} 4.2 Hz, H-3′), 4.05 (dd, 1 H, J_{6b,6a} 11.9, J_{6b,5} 5.0 Hz, H-6b), 3.72-3.80 (m, 4 H, H-4, OCH₃), 3.62 (dd, 1 H, J_{6a′,6b′} 9.4, J_{6a′,5′} 5.5 Hz, H-6a′), 3.53 (ddd, 1 H, J_{5,4} 9.9, J_{5,6b} 5.0, J_{5,6a} 2.0 Hz, H-5), 3.45 (dd, 1 H, J_{6b′,6a′} 9.4, J_{6b′,5′} 7.0 Hz, H-6b′), 2.91 (ddd, 1 H, J_{5′,6b′} 7.0, J_{5′,6a′} 5.5, J_{5′,4′} 1.1 Hz, H-5′), 2.09, 2.05, 2.01, 1.98, 1.97 (s, 15 H, 5×OAc).

Benzyl 2,2′,3,3′,6-penta-O-acetyl-4′-deoxy-6′-O-p-methoxybenzyl-,β-lactoside (28)

27 (0.50 g, 0.57 mmol) was dissolved in anhydrous benzene (15 mL), then tributyltin hydride (0.83 g, 2.87 mmol) and a catalytic amount of AIBN were added and the reaction mixture was refluxed under an atmosphere of argon. After 7 h, hexane (60 mL) was added and the reaction mixture was extracted with acetonitrile (80 mL). The acetonitrile layers were washed with two additional portions of hexane (60 mL) before the solvent was evaporated under reduced pressure. Chromatography over silica gel (PE:EtOAc, 2:1 to 3:2) yielded 28 (0.36 g, 84%) as a colourless gum. ¹H NMR (CDCl₃, 400 MHz): δ 7.15-7.35 (m, 7 H, Ar), 6.84-6.89 (m, 2 H, Ar), 5.11 (dd, 1 H, J_{3,2} 9.4, J_{3,4} 9.2 Hz, H-3), 4.94 (dd, 1 H, J_{2,3} 9.4, J_{2,1} 7.9 Hz, H-2), 4.89 (ddd, 1 H, J_{3′,4′ax} 11.5, J_{3′,2′} 9.7, J_{3′,4′eq} 5.4 Hz, H-3′), 4.82 (d, 1 H, J 12.3 Hz, PhCH), 4.76 (dd, 1 H, J_{2′,3′} 9.7, J_{2′,1′} 7.8 Hz, H-2′), 4.55 (d, 1 H, J 12.3 Hz, PhCH), 4.49 (dd, 1 H, J_{6a,6b} 12.0, J_{6a,5} 2.0 Hz, H-6a), 4.47 (d, 1 H, J_{1,2} 7.9 Hz, H-1), 4.41 (s, 2 H, MeOPhCH₂), 4.35 (d, 1 H, J_{1′,2′} 7.8 Hz, H-1′), 4.10 (dd, 1 H, J_{6b,6a} 12.0, J_{6b,5} 4.9 Hz, H-6b), 3.73-3.80 (m, 4 H, H-4, OCH₃), 3.51-3.64 (m, 2 H, H-5, H-5′), 3.51 (dd, 1 H, J_{6a′,6b′} 9.8, J_{6a′,5′} 5.1 Hz, H-6a′), 3.40 (dd, 1 H, J_{6b′,6a′} 9.8, J_{6b′,5′} 5.0 Hz, H-6b′), 2.09,2.01, 1.97, 1.95 (s, 15 H, 5×OAc), 1.47-1.63 (m, 2 H, H-4′ₐₓ, H-4′_{eq}).

Benzyl 2,2′,3,3′,6-penta-O-acetyl-4′-deoxy-β-lactoside (29)

Ceric ammonium nitrate (0.55 g, 1.00 mmol) was added to a solution of 28 (0.34 g, 0.46 mmol) in 9:1 acetonitrile/water (4 mL) and the reaction mixture was stirred at room temperature for 6 h before being added to aq. NaHCO₃ (15 mL) and extracted with CH₂Cl₂ (3×15 mL). The combined organic extracts were washed with aq. NaHCO₃ (15 mL), water (15 mL) and brine (15 mL), dried over MgSO₄ and the solvent was evaporated under reduced pressure. The resulting residue was crystallized from EtOAc/Hex to yield 29 (232 mg, 81%) as a white needle-like solid. ¹H NMR (CDCl₃, 400 MHz): δ 7.20-7.35 (m, 5 H, Ar), 5.14 (dd, 1 H, J_{3,2} 92, J_{3,4} 9.1 Hz, H-3), 4.94 (dd, 1H, J_{2,3} 9.2, J_{2,1} 7.8 Hz, H-2), 4.88-4.96 (m, 1 H, H-3′), 4.83 (d, 1 H, J 12.3 Hz, PhCH), 4.78 (dd, 1 H, J_{2′,3′} 9.4, J_{2′,1′} 7.7 Hz, H-2′), 4.57 (d, 1 H, J 12.3 Hz, PhCH), 4.51 (dd, 1 H, J_{6a,6b} 11.8, J_{6a,5} 2.1 Hz, H-6a), 4.49 (d, 1 H, J_{1,2} 7.8 Hz, H -1), 4.46 (d, 1 H, J_{1′,2′} 7.7 Hz, H-1′), 4.08 (dd, 1 H, J_{6b,6a} 11.8, J_{6b,5} 5.3 Hz, H-6b), 3.82 (dd, 1 H, J_{4,5} 9.5, J_{4,3} 9.1 Hz, H-4), 3.51-3.64 (m, 4 H, H-5, H-5′, H-6a′, H-6b′), 2.11, 2.04, 2.03, 1.98, 1.97 (s, 15 H, 5×OAc), 1.46-1.60 (m, 2 H, H-4′ₐₓ, H-4′_{eq}).

Anal. Calcd. for C₂₉H₃₈O₁₅: C, 55.59; H, 6.11. Found: C, 55.91; H, 6.16.

Benzyl 4′-deoxy-β-lactoside (30)

A solution of 29 (0.21 g, 0.33 mmol) in anhydrous MeOH (10 mL) under argon was made basic through the addition of a catalytic amount of sodium methoxide. The reaction mixture was stirred at room temperature overnight before it was neutralized with acidic Amberlyte® resin. Removal of the resin by filtration followed by evaporation of the solvent in vacuo gave a quantitative yield of 30 (147 mg) as a white solid. m.p 169-171° C. ¹H NMR (CD₃OD, 400 MHz) selected data only: δ 7.22-7.45 (m, 5 H, Ar), 4.91 (d, 1H, J 11.8 Hz, PhCH), 4.66 (d, 1 H, J 11.8 Hz, PhCH), 4.39 (d, 1 H, J_{1,2} 7.8 Hz, H-1), 4.34 (d, 1 H, J_{1′,2′} 7.8 Hz, H-1′), 3.93 (dd, 1 H, J_{6a,6b} 12.1, J_{6a,5} 2.4 Hz, H-6a), 3.86 (dd, 1 H, J_{6b,6a} 12.1, J_{6b,5} 4.2 Hz, H-6b), 3.13 (dd, 1 H, J_{4,5} 8.7, J_{4,3} 8.1 Hz, H4), 1.89 (dd, 1 H, J_{4′eq,4′ax} 12.7, J_{4′eq,3′} 5.0 Hz, H-4′_{eq}), 1.41 (ddd, 1 H, J_{4′ax,4′eq} 12.7, J_{4′ax,3′} 11.9, J_{4′ax,5′} 11.9 Hz, H-4′ₐₓ).

Anal. Calcd. for C₁₉H₂₈O₁₀: C, 54.80; H, 6.78. Found: C, 54.50; H, 6.72.

Synthesis of ¹⁸O-UDPGal:

Diphenyl (2,3,4,6-tetra-O-acetyl)-[1-¹⁸O]-α-D-galactopyranosyl Phosphate (31)

To a solution of 2,3,4,6-tetra-O-acetyl-D-galactose (0.49 g, 1.41 mmol) in anhydrous acetonitrile (2 mL) in a thick walled bomb was added 97% ¹⁸O-enriched water (0.5 mL). A few beads of Amberlyte® IR-120 acidic resin were added and the chamber was flooded with argon and sealed. The reaction mixture was then heated to 105° C. for 24 h. After this time, the solvent was removed in vacuo and the residue was chromatographed over silica gel (PE:EtOAc, 3:2 to 1:1). This 1-¹⁸O-labeled 2,3,4,6-tetra-O-acetyl-D-galactopyranose along with DMAP (025 g, 2.03 mmol) were then dissolved in anhydrous CH₂Cl₂ and stirred at room temperature for 20 min under an atmosphere of argon. Diphenyl chlorophosphate (0.54 g, 2.03 mmol) was then added to the reaction mixture and stirring was continued for 3 h, when it was then added to a 10% v/v solution of HCl (40 mL) and extracted with CH₂Cl₂ (3×40 mL). The combined organic extracts were washed with aq. NaHCO₃ (35 mL) and water (3×35 mL), dried over MgSO₄ and the solvent was evaporated under reduced pressure. Chromatography over silica gel (PE:EtOAc, 12:7 to 3:2) yielded 31 (316.1 mg, 39%) as a colourless gum. ¹HNMR(CDCl₃, 400 MHz): δ 7.15-7.45 (m, 10 H, Ar), 6.10 (dd, 1 H, J_{1,F} 6.4, J_{1,2} 3.3 Hz, H-1), 5.47 (dd, 1 H, J_{4,3} 3.1, J_{4,5} 1.1 Hz, H-4), 5.37 (dd, 1 H, J_{3,2} 10.9, J_{3,4} 3.1 Hz, H-3), 5.23 (ddd, 1 H J_{2,3} 10.9, J_{2,1} 3.3, J_{2,P} 3.0 Hz, H-2), 4.32 (ddd, 1 H, J_{5,6a} 6.6, J_{5,6b} 6.5, J_{5,4} 1.1 Hz, H-5), 4.06 (dd, 1 H, J_{6a,6b} 11.3, J_{6a,5} 6.6 Hz, H-6a), 3.91 (dd, 1 H, J_{6b,6a} 11.3, J_{6b,5} 6.5 Hz, H-6b), 2.12, 1.97, 1.90, 1.83 (s, 12 H, 4×OAc). ³¹P NMR (CDCl₃, 81 MHz, proton decoupled): δ-13.70. LR-LSIMS: calcd. for C₂₆H₂₉O₁₃P: 581. Found: 581 (C₂₆H₂₉¹⁶O₁₃P), 583 (C₂₆H₂₉¹⁶O₁₂¹⁸OP), ¹⁶O /¹⁸O=20/80.

α-D-Galactopyranosyl-[1-¹⁸O]-phosphate Monopyridinium Salt (32)

31 (0.31 g, 0.54 mmol) was dissolved in 1:1 EtOAc:MeOH (6 mL) when PtO₂ (0.10 g) was added and the reaction mixture was hydrogenated at 6 atm. After 2 d, this mixture was filtered through Celite® and chromatographed over silica gel (EtOAc to EtOAc:MeOH:H₂O, 27:2:1 to 7:2:1). The desired fractions were pooled, concentrated and redissolved in THF (1 mL). To this was then added 2 M LiOH (2 mL) and the reaction mixture was allowed to stir at room temperature overnight. The reaction volume was then reduced and eluted through a Bio-Rad® AG 50W-X2, 200-400 mesh sulfonic acid cation exchange column (pyridinium form). The desired fractions were pooled and lyophilized to yield 32 (126 mg, 69%) as a white fluffy solid ¹H NMR (D₂O, 300 MHz): δ 7.90-8.70 (m, 5 H, pyridine), 5.40 (dd, 1 H, J_{1,P} 7.0, J_{3,2} 3.5 Hz, H-1), 4.00 (dd, 1 H, J_{5,6a} 6.3, J_{5,6b} 6.3 Hz, H-5), 3.88 (d, 1 H, J_{4,3} 2.9 Hz, H-4), 3.77 (dd, 1 H, J_{3,2} 10.3, J_{3,4} 2.9 Hz, H-3), 3.68 (ddd, 1 H, J_{2,3} 10.3, J_{2,1} 3.5, J_{2,P} 3.0 Hz, H-2), 3.53-3.63 (m, 2 H, H-6a, H-6b). ³¹P NMR (D₂O, 121 MHz, proton decoupled): δ 0.09. LR-LSIMS: calcd for C₆H₁₂O₉P-: 259. Found: 259 (C₆H₁₂¹⁶O₉P⁻), 261 (C₆H₁₂¹⁶O₈¹⁸OP⁻), ¹⁶O/¹⁸O=17/83.

Uridine 5′-diphospho-[1″-¹⁸O-α-D-galactopyranose, Diammonium Salt (33)

To a solution of 32 (0.08 g, 0.24 mmol) in water (1.5 mL) was added tri-n-octylamine (0.10 mL, 0.24 mmol) and the mixture was lyophilized. The resulting residue along with UMP-morpholidate (0.18 g, 0.26 mmol) was dried over P₂O₅ overnight. The two reagents were then dissolved in anhydrous pyridine (2.5 mL) and the reaction mixture was stirred at room temperature in the presence of 4 Å molecular sieves under an argon atmosphere. An aliquot of the reaction mixture was transferred into an NMR tube containing a capillary of DMSO-d6 so that the reaction progress can be monitored by ³¹P NMR. After 11 d, the mixture was filtered and the filtrate was added to 50 mM NH₄HCO₃. The tri-n-octylamine was extracted with Et₂O (3×15 mL) and the aqueous phase was lyophilized to yield the crude product. Purification was afforded by anion exchange chromatography on a DEAE Sephacel column (26 mm×12.5 cm, 50-500 mM NH₄HCO₃, 1.5 mL/min) followed by size exclusion chromatography on a Bio-Gel P2, extra fine column (16 mm×55 cm, 50 mM NH₄HCO₃, 0.15 mL/min) using a Beckman Biosepra ProSys Workstation. The desired fractions were pooled and lyophilized to yield 33 (25 mg, 18%) as a white powder. ¹H NMR (D₂O, 300 MHz): δ 7.83 (d, 1 H, J_{6,5} 8.2 Hz, H-6), 5.30-5.40 (m, 2 H, H-5, H-1′), 5.53 (dd, 1 H, J_{1″,P} 7.2, J_{1″,2″} 3.6 Hz, H-1″), 4.23-4.28 (m, 2 H, H-2′, H-3′), 4.07-4.20 (m, 3 H, H4′, H-5a′, H-5b′), 4.06 (dd, 1 H, J_{5″,6a″} 6.2, J_{5″, 6b″} 6.2 Hz, H-5″), 3.92 (d, 1 H, J_{4″,3″} 3.0 Hz, H-4″), 3.80 (dd, 1 H, J_{3″,2″} 10.2, J_{3″,4″} 3.0 Hz, H-3″), 3.68 (ddd, 1 H, J_{2″,3″} 10.2, J_{2″,1″} 3.6, J_{2″,P} 3.0 Hz, H-2″), 3.55-3.65 (m, 2 H, H-6a″, H-6b″). ³¹P NMR (D₂O, 121 MHz, proton decoupled): δ-9.95 (d, 1P, J_{Pβ,Pα} 20.1 Hz, P^{β}), -11.51 (d, 1P, J_{Pα,Pβ} 20.7 Hz, P^{α}). HR-LSIMS: calcd. for C₁₅H₂₃N₂¹⁶O₁₆¹⁸OP₂⁻: 567.0513. Found 567.0515. LR-LSIMS: ¹⁶O/¹⁸O=15/85.

Anal. Calcd. for C₁₅H₂₂N₂¹⁶O₁₆¹⁸OP₂²⁻.2NH₄⁺: C, 29.86; H, 5.18; N, 9.29. Found: C, 30.21; H, 5.16; N, 8.97.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

TABLE 1
Kinetic parameters of wild-type LgtC and its
various mutants for its substrates UDP-Gal and lactose.

| | UDP-Gal² | | | Lactose³ | | |
|---|---|---|---|---|---|---|
| | k_{cat} | Kₘ | | k_{cat} | Kₘ | k_{cat}/Kₘ |
| LgtC¹ | (s)⁻¹ | (μM) | k_{cat}/K_{m (μM}⁻¹s⁻¹) | (s)⁻¹ | (mM) | (mM⁻¹s⁻¹) |
| WT | 14.2 | 18 | 0.81 | 24.0 | 20 | 1.20 |
| C128/174S | 11.4 | 12 | 0.97 | 19.2 | 13 | 1.48 |
| D103E | 0.004 | 218 | 0.00002 | 0.003 | 34 | 0.00009 |
| D103N | 0.006 | 187 | 0.00003 | 0.006 | 58 | 0.00010 |
| Y186F | 25.6 | 15 | 1.67 | 31.6 | 16 | 1.92 |
| Q189A | 0.4 | 25 | 0.017 | 1.0 | 136 | 0.0074 |

¹All constructs in this table have the C-terminal 25 residues deleted.
²kinetic parameters were determined at a constant lactose concentration of 100 mM
³kinetic parameters were determined at a constant UDP-Gal concentration of 250 μM

TABLE 2
Data collection and refinement statistics¹
Data collection

| | | | | | 4-deoxy- |
|---|---|---|---|---|---|
| | Peak | Inflection | Remote1 | Remote2 | lactose |
| Resolution (Å) | 20-2.0 | 20-2.0 | 20-2.0 | 20-2.0 | 20-2.0 |
| Wavelength | 0.97949 | 0.97996 | 0.92526 | 1.06883 | 1.5418 |
| (Å) | | | | | |
| Unique | 18498 | 18492 | 18400 | 18516 | 18851 |
| reflections | | | | | |
| Completeness | 99.0 | 99.0 | 98.9 | 97.2 | 98.3 |
| (%) | (97.7) | (97.7) | (97.8) | (83.9) | (90.2) |
| I/I(σ) | 19.8 | 19.9 | 19.1 | 19.7 | 20.7 |
| | (6.1) | (6.1) | (5.9) | (5.1) | (8.03) |
| Rmerge (%) | 4.4 | 4.4 | 4.7 | 4.2 | 6.2 |
| | (11.4) | (11.4) | (12.3) | (9.2) | (15.2) |

Refinement statistics and model stereochemistry

| | LgtC/Mn/donor | LgtC/Mn/donor/acceptor |
|---|---|---|
| Resolution (Å) | 20-2.0 | 20-2.0 |
| Unique reflections | 18038 | 18225 |
| Rcryst/Rfree (%) | 19.9/22.74 | 19.3/22.76 |
| RMSD bonds (Å) | 0.007 | 0.007 |
| RMSD angles (°) | 1.31 | 1.33 |
| Average B-factor (Å²) | | |
| Protein | 13.5 | 16.3 |
| UDP 2-deoxy-2-fluoro- | 11.6 | 15.4 |
| galactose | | |
| 4-deoxylactose | | 19.8 |
| Mn | 5.3 | 11.3 |
| Water | 21.1 | 23.6 |
| Ramachandran plot | | |
| Residues in most favored | 92.2 | 91.0 |
| regions (%) | | |
| Additionally allowed | 7.0 | 8.6 |
| regions (%) | | |
| Generously allowed regions | 0.4 | 0.4 |
| (%) | | |
| Disallowed regions (%) | 0.4 | 0 |

¹Statistics for the highest resolution shell (2.07-2.0) are given in brackets.

TABLE 3
Atomic Interactions of a Retaining Glycosyltransferase with a
Sugar Nucleotide Donor and/or Acceptor Molecules

| | Atomic Contact on | | |
|---|---|---|---|
| | Sugar Nucleotide | | |
| No. of Atomic | Donor or Acceptor | | Binding Site |
| Interaction | Molecules | Atomic Contact on Enzyme | Property |
| 1 | Uracil O2 | Main chain nitrogen of Asp 8 | HB |
| 2 | Uracil N3 | OD1 of Asp 8 | HB |
| 3 | Uracil carbonyl 04 | ND2 group of Asn 10 | HB |
| 4 | Ribose ring 02 | Carbonyl oxygen of Ala 6 | |
| 5 | Ribose O3 | Main chain amide of Ile 104 | |
| 6 | Phosphate 02A | Lys 250 (NZ) | HB |
| 7 | Phosphate 02B | Gly 247 (N) and His 78 (NE2) | HB |
| 8 | Sugar donor ring O3 | Asp 103 and Arg 86 | HB |
| 9 | Sugar donor ring 04′ | Carboxylate of Asp 188 | HB |
| 10 | Sugar donor ring 06′ | Carboxylate of Asp 188 | HB |
| 11 | Mn²⁺ | His 244, Asp 103, | |
| | | Asp 105 | |
| 12 | Lactose 06 | Asp 130 (OD2) and Gln 189 (NE2) | HB |
| 13 | Lactose 06 | Side chain atoms of Val 76, His 78, Tyr | VdW |
| | | 186, Cys 246, and Gly 247 | |
| 14 | Reducing end glucose | Phe 132 | HP |
| | moiety of lactose | | |
| 15 | | Pro 211 and Pro 248 | VdW |
| 16 | Reducing end glucose | Thr 212 hydroxyl, main chain nitrogen | HB |
| | moiety of lactose 03′ | | |
| 17 | | Cys 246 | HB |

HB: hydrogen bond interaction
HP: hydrophobic
VdW van der Waals interaction

| REMARK | coordinates from minimization refinement | | |
|---|---|---|---|
| REMARK | refinement resolution: 20.0-2.0 A | | |
| REMARK | starting r = .2046 free_r = .2331 | | |
| REMARK | final r = .2033 free_r = .2321 | | |
| REMARK | rmsd bonds = .005248 rmsd angles = 1.22351 | | |
| REMARK | wa = .890542 | | |
| REMARK | target = mlf cycles = 1 steps = 100 | | |
| REMARK | sg = P2 (1) 2 (1) 2 (1) a = 39.79 b = 76.05 c = 86.84 alpha = 90 beta = 90 gamma = 90 | | |
| REMARK | parameter file 1: CNS_TOPPAR:protein_rep.param | | |
| REMARK | parameter file 2: ../rnd4/upg.par | | |
| REMARK | parameter file 3: CNS_TOPPAR:ion.param | | |
| REMARK | parameter file 4: CNS_TOPPAR:water_rep.param | | |
| REMARK | parameter file 5: ../rnd6/acy.par | | |
| REMARK | molecular structure file: generate7_2.mtf | | |
| REMARK | input coordinates: generate7_2.pdb | | |
| REMARK | reflection file = ../lgtC.cv | | |
| REMARK | ncs = none | | |
| REMARK | B-correction resolution: 6.0-2.0 | | |
| REMARK | initial B-factor correction applied to fobs: | | |
| REMARK | B11 = 1.111 B22 = 2.062 B33 = −3.173 | | |
| REMARK | B12 = .000 B13 = .000 B23 = .000 | | |
| REMARK | B-factor correction applied to coordinate array B: .174 | | |
| REMARK | bulk solvent: density level = .335425 e/A\hat{ }3, B-factor = 42.4467 A\hat{ }2 | | |
| REMARK | reflections with \|Fobs\|/sigma_F < 0.0 rejected | | |
| REMARK | reflections with \|Fobs\| > 10000 * rms (Fobs) rejected | | |
| REMARK | theoretical total number of refl. in resol. range: | 18411 | (100.0%) |
| REMARK | number of unobserved reflections (no entry or \|F\|= 0): | 373 | (2.0%) |
| REMARK | number of reflections rejected: | 0 | (>.0%) |
| REMARK | total number of reflections used: | 18038 | (>98.0%) |
| REMARK | number of reflections in working set: | 17161 | (>93.2%) |
| REMARK | number of reflections in test set: | 877 | (>4.8%) |

| | |
|---|---|
| CRYST1 | 39.790 76.050 86.840 90.00 90.00 90.00 P 21 21 21 |
| REMARK | FILENAME = “minimize7_3.pdb” |
| REMARK | DATE: 29-May-00 21:53:49 created by user: karina |
| REMARK | VERSION: 1.0 |

| | | | | | | | | | | |
|---|---|---|---|---|---|---|---|---|---|---|
| ATOM | 1 | CB | MSE | 1 | 47.916 | 46.223 | 65.035 | 1.00 | 15.18 | DIC |
| ATOM | 2 | CG | MSE | 1 | 47.459 | 47.194 | 63.956 | 1.00 | 18.95 | DIC |
| ATOM | 3 | SE | MSE | 1 | 46.533 | 48.765 | 64.672 | 1.00 | 23.16 | DIC |
| ATOM | 4 | CE | MSE | 1 | 48.073 | 49.691 | 65.392 | 1.00 | 19.79 | DIC |
| ATOM | 5 | C | MSE | 1 | 47.671 | 44.106 | 63.711 | 1.00 | 10.02 | DIC |
| ATOM | 6 | O | MSE | 1 | 47.762 | 44.051 | 62.487 | 1.00 | 9.02 | DIC |
| ATOM | 7 | N | MSE | 1 | 49.817 | 45.335 | 63.713 | 1.00 | 9.31 | DIC |
| ATOM | 8 | CA | MSE | 1 | 48.632 | 44.961 | 64.526 | 1.00 | 10.07 | DIC |
| ATOM | 9 | N | ASP | 2 | 46.762 | 43.426 | 64.398 | 1.00 | 8.25 | DIC |
| ATOM | 10 | CA | ASP | 2 | 45.786 | 42.580 | 63.729 | 1.00 | 8.29 | DIC |
| ATOM | 11 | CB | ASP | 2 | 45.742 | 41.193 | 64.376 | 1.00 | 8.73 | DIC |
| ATOM | 12 | CG | ASP | 2 | 47.016 | 40.405 | 64.139 | 1.00 | 11.00 | DIC |
| ATOM | 13 | OD1 | ASP | 2 | 47.966 | 40.549 | 64.934 | 1.00 | 9.98 | DIC |
| ATOM | 14 | OD2 | ASP | 2 | 47.073 | 39.655 | 63.139 | 1.00 | 12.73 | DIC |
| ATOM | 15 | C | ASP | 2 | 44.413 | 43.224 | 63.763 | 1.00 | 6.99 | DIC |
| ATOM | 16 | O | ASP | 2 | 43.839 | 43.440 | 64.831 | 1.00 | 5.46 | DIC |
| ATOM | 17 | N | ILE | 3 | 43.906 | 43.535 | 62.573 | 1.00 | 7.26 | DIC |
| ATOM | 18 | CA | ILE | 3 | 42.604 | 44.172 | 62.399 | 1.00 | 5.51 | DIC |
| ATOM | 19 | CB | ILE | 3 | 42.680 | 45.315 | 61.356 | 1.00 | 6.61 | DIC |
| ATOM | 20 | CG2 | ILE | 3 | 41.276 | 45.917 | 61.119 | 1.00 | 3.06 | DIC |
| ATOM | 21 | CG1 | ILE | 3 | 43.692 | 46.372 | 61.806 | 1.00 | 4.70 | DIC |
| ATOM | 22 | CD | ILE | 3 | 43.312 | 47.108 | 63.082 | 1.00 | 7.67 | DIC |
| ATOM | 23 | C | ILE | 3 | 41.585 | 43.166 | 61.893 | 1.00 | 6.14 | DIC |
| ATOM | 24 | O | ILE | 3 | 41.881 | 42.379 | 60.990 | 1.00 | 6.52 | DIC |
| ATOM | 25 | N | VAL | 4 | 40.388 | 43.205 | 62.472 | 1.00 | 5.89 | DIC |
| ATOM | 26 | CA | VAL | 4 | 39.302 | 42.322 | 62.072 | 1.00 | 4.89 | DIC |
| ATOM | 27 | CB | VAL | 4 | 38.791 | 41.468 | 63.248 | 1.00 | 6.30 | DIC |
| ATOM | 28 | CG1 | VAL | 4 | 37.584 | 40.638 | 62.795 | 1.00 | 8.75 | DIC |
| ATOM | 29 | CG2 | VAL | 4 | 39.904 | 40.558 | 63.753 | 1.00 | 5.68 | DIC |
| ATOM | 30 | C | VAL | 4 | 38.115 | 43.110 | 61.519 | 1.00 | 5.54 | DIC |
| ATOM | 31 | O | VAL | 4 | 37.688 | 44.111 | 62.097 | 1.00 | 4.90 | DIC |
| ATOM | 32 | N | PHE | 5 | 37.603 | 42.655 | 60.382 | 1.00 | 5.50 | DIC |
| ATOM | 33 | CA | PHE | 5 | 36.449 | 43.260 | 59.728 | 1.00 | 5.20 | DIC |
| ATOM | 34 | CB | PHE | 5 | 36.832 | 43.859 | 58.364 | 1.00 | 5.40 | DIC |
| ATOM | 35 | CG | PHE | 5 | 37.439 | 45.234 | 58.430 | 1.00 | 4.45 | DIC |
| ATOM | 36 | CD1 | PHE | 5 | 36.693 | 46.323 | 58.876 | 1.00 | 4.41 | DIC |
| ATOM | 37 | CD2 | PHE | 5 | 38.732 | 45.453 | 57.969 | 1.00 | 5.91 | DIC |
| ATOM | 38 | CE1 | PHE | 5 | 37.228 | 47.619 | 58.851 | 1.00 | 6.66 | DIC |
| ATOM | 39 | CE2 | PHE | 5 | 39.276 | 46.743 | 57.940 | 1.00 | 7.08 | DIC |
| ATOM | 40 | CZ | PHE | 5 | 38.516 | 47.828 | 58.381 | 1.00 | 6.75 | DIC |
| ATOM | 41 | C | PHE | 5 | 35.501 | 42.090 | 59.479 | 1.00 | 6.22 | DIC |
| ATOM | 42 | O | PHE | 5 | 35.929 | 40.934 | 59.475 | 1.00 | 6.65 | DIC |
| ATOM | 43 | N | ALA | 6 | 34.222 | 42.390 | 59.282 | 1.00 | 4.81 | DIC |
| ATOM | 44 | CA | ALA | 6 | 33.221 | 41.372 | 58.988 | 1.00 | 4.67 | DIC |
| ATOM | 45 | CB | ALA | 6 | 32.352 | 41.084 | 60.213 | 1.00 | 2.66 | DIC |
| ATOM | 46 | C | ALA | 6 | 32.366 | 41.944 | 57.872 | 1.00 | 5.29 | DIC |
| ATOM | 47 | O | ALA | 6 | 31.920 | 43.090 | 57.952 | 1.00 | 4.39 | DIC |
| ATOM | 48 | N | ALA | 7 | 32.132 | 41.164 | 56.826 | 1.00 | 5.20 | DIC |
| ATOM | 49 | CA | ALA | 7 | 31.314 | 41.669 | 55.736 | 1.00 | 6.73 | DIC |
| ATOM | 50 | CB | ALA | 7 | 32.120 | 42.659 | 54.901 | 1.00 | 8.23 | DIC |
| ATOM | 51 | C | ALA | 7 | 30.755 | 40.590 | 54.834 | 1.00 | 8.12 | DIC |
| ATOM | 52 | O | ALA | 7 | 31.340 | 39.515 | 54.695 | 1.00 | 7.34 | DIC |
| ATOM | 53 | N | ASP | 8 | 29.598 | 40.885 | 54.247 | 1.00 | 8.39 | DIC |
| ATOM | 54 | CA | ASP | 8 | 28.967 | 39.987 | 53.295 | 1.00 | 8.38 | DIC |
| ATOM | 55 | CB | ASP | 8 | 27.449 | 39.915 | 53.502 | 1.00 | 9.33 | DIC |
| ATOM | 56 | CG | ASP | 8 | 26.832 | 41.261 | 53.816 | 1.00 | 9.71 | DIC |
| ATOM | 57 | OD1 | ASP | 8 | 27.334 | 42.290 | 53.312 | 1.00 | 8.87 | DIC |
| ATOM | 58 | OD2 | ASP | 8 | 25.828 | 41.285 | 54.561 | 1.00 | 9.54 | DIC |
| ATOM | 59 | C | ASP | 8 | 29.294 | 40.608 | 51.941 | 1.00 | 9.19 | DIC |
| ATOM | 60 | O | ASP | 8 | 29.926 | 41.664 | 51.878 | 1.00 | 7.12 | DIC |
| ATOM | 61 | N | ASP | 9 | 28.873 | 39.969 | 50.859 | 1.00 | 9.74 | DIC |
| ATOM | 62 | CA | ASP | 9 | 29.180 | 40.485 | 49.532 | 1.00 | 10.05 | DIC |
| ATOM | 63 | CB | ASP | 9 | 28.551 | 39.582 | 48.471 | 1.00 | 10.66 | DIC |
| ATOM | 64 | CG | ASP | 9 | 29.132 | 39.817 | 47.098 | 1.00 | 10.96 | DIC |
| ATOM | 65 | OD1 | ASP | 9 | 30.353 | 39.613 | 46.927 | 1.00 | 9.95 | DIC |
| ATOM | 66 | OD2 | ASP | 9 | 28.370 | 40.210 | 46.192 | 1.00 | 15.56 | DIC |
| ATOM | 67 | C | ASP | 9 | 28.731 | 41.935 | 49.328 | 1.00 | 11.33 | DIC |
| ATOM | 68 | O | ASP | 9 | 29.427 | 42.729 | 48.689 | 1.00 | 11.67 | DIC |
| ATOM | 69 | N | ASN | 10 | 27.578 | 42.282 | 49.889 | 1.00 | 11.40 | DIC |
| ATOM | 70 | CA | ASN | 10 | 27.033 | 43.626 | 49.760 | 1.00 | 12.66 | DIC |
| ATOM | 71 | CB | ASN | 10 | 25.707 | 43.721 | 50.518 | 1.00 | 14.73 | DIC |
| ATOM | 72 | CG | ASN | 10 | 25.079 | 45.098 | 50.424 | 1.00 | 16.23 | DIC |
| ATOM | 73 | OD1 | ASN | 10 | 24.924 | 45.644 | 49.335 | 1.00 | 16.14 | DIC |
| ATOM | 74 | ND2 | ASN | 10 | 24.706 | 45.662 | 51.569 | 1.00 | 15.48 | DIC |
| ATOM | 75 | C | ASN | 10 | 27.972 | 44.730 | 50.244 | 1.00 | 12.57 | DIC |
| ATOM | 76 | O | ASN | 10 | 27.962 | 45.839 | 49.705 | 1.00 | 12.93 | DIC |
| ATOM | 77 | N | TYR | 11 | 28.782 | 44.432 | 51.254 | 1.00 | 10.65 | DIC |
| ATOM | 78 | CA | TYR | 11 | 29.702 | 45.425 | 51.805 | 1.00 | 10.78 | DIC |
| ATOM | 79 | CB | TYR | 11 | 29.632 | 45.399 | 53.336 | 1.00 | 10.58 | DIC |
| ATOM | 80 | CG | TYR | 11 | 28.703 | 46.427 | 53.944 | 1.00 | 11.86 | DIC |
| ATOM | 81 | CD1 | TYR | 11 | 27.599 | 46.915 | 53.239 | 1.00 | 11.68 | DIC |
| ATOM | 82 | CE1 | TYR | 11 | 26.737 | 47.853 | 53.811 | 1.00 | 12.47 | DIC |
| ATOM | 83 | CD2 | TYR | 11 | 28.918 | 46.900 | 55.239 | 1.00 | 11.28 | DIC |
| ATOM | 84 | CE2 | TYR | 11 | 28.063 | 47.829 | 55.818 | 1.00 | 13.50 | DIC |
| ATOM | 85 | CZ | TYR | 11 | 26.978 | 48.301 | 55.101 | 1.00 | 12.90 | DIC |
| ATOM | 86 | OH | TYR | 11 | 26.144 | 49.225 | 55.681 | 1.00 | 15.54 | DIC |
| ATOM | 87 | C | TYR | 11 | 31.155 | 45.267 | 51.369 | 1.00 | 11.16 | DIC |
| ATOM | 88 | O | TYR | 11 | 32.043 | 45.919 | 51.925 | 1.00 | 11.02 | DIC |
| ATOM | 89 | N | ALA | 12 | 31.400 | 44.421 | 50.371 | 1.00 | 10.59 | DIC |
| ATOM | 90 | CA | ALA | 12 | 32.760 | 44.183 | 49.892 | 1.00 | 10.32 | DIC |
| ATOM | 91 | CB | ALA | 12 | 32.744 | 43.157 | 48.754 | 1.00 | 11.38 | DIC |
| ATOM | 92 | C | ALA | 12 | 33.489 | 45.450 | 49.441 | 1.00 | 9.91 | DIC |
| ATOM | 93 | O | ALA | 12 | 34.660 | 45.647 | 49.766 | 1.00 | 9.42 | DIC |
| ATOM | 94 | N | ALA | 13 | 32.804 | 46.306 | 48.688 | 1.00 | 9.60 | DIC |
| ATOM | 95 | CA | ALA | 13 | 33.414 | 47.540 | 48.205 | 1.00 | 9.28 | DIC |
| ATOM | 96 | CB | ALA | 13 | 32.481 | 48.229 | 47.204 | 1.00 | 9.14 | DIC |
| ATOM | 97 | C | ALA | 13 | 33.756 | 48.491 | 49.355 | 1.00 | 8.84 | DIC |
| ATOM | 98 | O | ALA | 13 | 34.815 | 49.125 | 49.357 | 1.00 | 8.22 | DIC |
| ATOM | 99 | N | TYR | 14 | 32.865 | 48.582 | 50.336 | 1.00 | 8.07 | DIC |
| ATOM | 100 | CA | TYR | 14 | 33.090 | 49.462 | 51.478 | 1.00 | 8.03 | DIC |
| ATOM | 101 | CB | TYR | 14 | 31.803 | 49.606 | 52.291 | 1.00 | 8.61 | DIC |
| ATOM | 102 | CG | TYR | 14 | 30.600 | 49.939 | 51.438 | 1.00 | 10.64 | DIC |
| ATOM | 103 | CD1 | TYR | 14 | 30.700 | 50.855 | 50.394 | 1.00 | 11.14 | DIC |
| ATOM | 104 | CE1 | TYR | 14 | 29.602 | 51.168 | 49.602 | 1.00 | 13.63 | DIC |
| ATOM | 105 | CD2 | TYR | 14 | 29.363 | 49.340 | 51.676 | 1.00 | 11.59 | DIC |
| ATOM | 106 | CE2 | TYR | 14 | 28.252 | 49.647 | 50.891 | 1.00 | 14.49 | DIC |
| ATOM | 107 | CZ | TYR | 14 | 28.384 | 50.563 | 49.854 | 1.00 | 14.26 | DIC |
| ATOM | 108 | OH | TYR | 14 | 27.302 | 50.879 | 49.068 | 1.00 | 17.66 | DIC |
| ATOM | 109 | C | TYR | 14 | 34.222 | 48.934 | 52.356 | 1.00 | 7.50 | DIC |
| ATOM | 110 | O | TYR | 14 | 34.995 | 49.710 | 52.914 | 1.00 | 8.49 | DIC |
| ATOM | 111 | N | LEU | 15 | 34.310 | 47.612 | 52.469 | 1.00 | 7.68 | DIC |
| ATOM | 112 | CA | LEU | 15 | 35.363 | 46.965 | 53.247 | 1.00 | 7.83 | DIC |
| ATOM | 113 | CB | LEU | 15 | 35.267 | 45.440 | 53.105 | 1.00 | 7.70 | DIC |
| ATOM | 114 | CG | LEU | 15 | 36.519 | 44.619 | 53.441 | 1.00 | 8.87 | DIC |
| ATOM | 115 | CD1 | LEU | 15 | 36.881 | 44.790 | 54.913 | 1.00 | 7.99 | DIC |
| ATOM | 116 | CD2 | LEU | 15 | 36.265 | 43.146 | 53.117 | 1.00 | 8.32 | DIC |
| ATOM | 117 | C | LEU | 15 | 36.719 | 47.428 | 52.729 | 1.00 | 6.26 | DIC |
| ATOM | 118 | O | LEU | 15 | 37.605 | 47.790 | 53.503 | 1.00 | 5.67 | DIC |
| ATOM | 119 | N | CYS | 16 | 36.868 | 47.407 | 51.407 | 1.00 | 6.41 | DIC |
| ATOM | 120 | CA | CYS | 16 | 38.109 | 47.814 | 50.761 | 1.00 | 6.53 | DIC |
| ATOM | 121 | CB | CYS | 16 | 37.972 | 47.683 | 49.241 | 1.00 | 7.09 | DIC |
| ATOM | 122 | SG | CYS | 16 | 39.490 | 48.066 | 48.352 | 1.00 | 10.90 | DIC |
| ATOM | 123 | C | CYS | 16 | 38.509 | 49.245 | 51.122 | 1.00 | 6.22 | DIC |
| ATOM | 124 | O | CYS | 16 | 39.673 | 49.521 | 51.415 | 1.00 | 6.80 | DIC |
| ATOM | 125 | N | VAL | 17 | 37.545 | 50.156 | 51.101 | 1.00 | 6.94 | DIC |
| ATOM | 126 | CA | VAL | 17 | 37.823 | 51.548 | 51.436 | 1.00 | 7.93 | DIC |
| ATOM | 127 | CB | VAL | 17 | 36.583 | 52.436 | 51.173 | 1.00 | 8.98 | DIC |
| ATOM | 128 | CG1 | VAL | 17 | 36.840 | 53.854 | 51.651 | 1.00 | 8.40 | DIC |
| ATOM | 129 | CG2 | VAL | 17 | 36.260 | 52.436 | 49.679 | 1.00 | 9.77 | DIC |
| ATOM | 130 | C | VAL | 17 | 38.240 | 51.670 | 52.900 | 1.00 | 7.79 | DIC |
| ATOM | 131 | O | VAL | 17 | 39.233 | 52.316 | 53.217 | 1.00 | 7.46 | DIC |
| ATOM | 132 | N | ALA | 18 | 37.481 | 51.041 | 53.790 | 1.00 | 7.51 | DIC |
| ATOM | 133 | CA | ALA | 18 | 37.789 | 51.091 | 55.213 | 1.00 | 7.78 | DIC |
| ATOM | 134 | CB | ALA | 18 | 36.750 | 50.295 | 56.005 | 1.00 | 7.26 | DIC |
| ATOM | 135 | C | ALA | 18 | 39.188 | 50.530 | 55.471 | 1.00 | 7.32 | DIC |
| ATOM | 136 | O | ALA | 18 | 39.978 | 51.125 | 56.201 | 1.00 | 8.92 | DIC |
| ATOM | 137 | N | ALA | 19 | 39.491 | 49.387 | 54.864 | 1.00 | 6.80 | DIC |
| ATOM | 138 | CA | ALA | 19 | 40.796 | 48.761 | 55.046 | 1.00 | 6.34 | DIC |
| ATOM | 139 | CB | ALA | 19 | 40.866 | 47.448 | 54.275 | 1.00 | 7.20 | DIC |
| ATOM | 140 | C | ALA | 19 | 41.904 | 49.698 | 54.585 | 1.00 | 9.12 | DIC |
| ATOM | 141 | O | ALA | 19 | 42.917 | 49.854 | 55.267 | 1.00 | 7.42 | DIC |
| ATOM | 142 | N | LYS | 20 | 41.714 | 50.333 | 53.431 | 1.00 | 9.41 | DIC |
| ATOM | 143 | CA | LYS | 20 | 42.736 | 51.243 | 52.937 | 1.00 | 10.30 | DIC |
| ATOM | 144 | CB | LYS | 20 | 42.404 | 51.725 | 51.523 | 1.00 | 11.46 | DIC |
| ATOM | 145 | CG | LYS | 20 | 43.560 | 52.483 | 50.883 | 1.00 | 17.23 | DIC |
| ATOM | 146 | CD | LYS | 20 | 43.346 | 52.750 | 49.404 | 1.00 | 19.42 | DIC |
| ATOM | 147 | CE | LYS | 20 | 44.561 | 53.444 | 48.812 | 1.00 | 21.28 | DIC |
| ATOM | 148 | NZ | LYS | 20 | 44.387 | 53.756 | 47.368 | 1.00 | 22.28 | DIC |
| ATOM | 149 | C | LYS | 20 | 42.912 | 52.435 | 53.881 | 1.00 | 10.50 | DIC |
| ATOM | 150 | O | LYS | 20 | 44.017 | 52.950 | 54.027 | 1.00 | 8.75 | DIC |
| ATOM | 151 | N | SER | 21 | 41.836 | 52.872 | 54.533 | 1.00 | 9.94 | DIC |
| ATOM | 152 | CA | SER | 21 | 41.959 | 53.997 | 55.459 | 1.00 | 9.54 | DIC |
| ATOM | 153 | CB | SER | 21 | 40.586 | 54.438 | 55.988 | 1.00 | 10.25 | DIC |
| ATOM | 154 | OG | SER | 21 | 40.096 | 53.556 | 56.983 | 1.00 | 11.69 | DIC |
| ATOM | 155 | C | SER | 21 | 42.859 | 53.588 | 56.626 | 1.00 | 7.93 | DIC |
| ATOM | 156 | O | SER | 21 | 43.587 | 54.411 | 57.172 | 1.00 | 8.07 | DIC |
| ATOM | 157 | N | VAL | 22 | 42.807 | 52.314 | 57.006 | 1.00 | 8.61 | DIC |
| ATOM | 158 | CA | VAL | 22 | 43.639 | 51.817 | 58.102 | 1.00 | 7.31 | DIC |
| ATOM | 159 | CB | VAL | 22 | 43.225 | 50.397 | 58.540 | 1.00 | 7.87 | DIC |
| ATOM | 160 | CG1 | VAL | 22 | 44.163 | 49.901 | 59.630 | 1.00 | 7.62 | DIC |
| ATOM | 161 | CG2 | VAL | 22 | 41.792 | 50.404 | 59.043 | 1.00 | 7.19 | DIC |
| ATOM | 162 | C | VAL | 22 | 45.107 | 51.785 | 57.684 | 1.00 | 8.07 | DIC |
| ATOM | 163 | O | VAL | 22 | 45.992 | 52.151 | 58.460 | 1.00 | 8.06 | DIC |
| ATOM | 164 | N | GLU | 23 | 45.371 | 51.340 | 56.460 | 1.00 | 7.76 | DIC |
| ATOM | 165 | CA | GLU | 23 | 46.749 | 51.296 | 55.977 | 1.00 | 9.02 | DIC |
| ATOM | 166 | CB | GLU | 23 | 46.832 | 50.605 | 54.609 | 1.00 | 9.88 | DIC |
| ATOM | 167 | CG | GLU | 23 | 46.444 | 49.125 | 54.602 | 1.00 | 11.02 | DIC |
| ATOM | 168 | CD | GLU | 23 | 46.763 | 48.450 | 53.276 | 1.00 | 13.23 | DIC |
| ATOM | 169 | OE1 | GLU | 23 | 46.541 | 49.086 | 52.228 | 1.00 | 15.00 | DIC |
| ATOM | 170 | OE2 | GLU | 23 | 47.221 | 47.285 | 53.279 | 1.00 | 12.64 | DIC |
| ATOM | 171 | C | GLU | 23 | 47.309 | 52.714 | 55.858 | 1.00 | 8.76 | DIC |
| ATOM | 172 | O | GLU | 23 | 48.447 | 52.976 | 56.246 | 1.00 | 8.97 | DIC |
| ATOM | 173 | N | ALA | 24 | 46.503 | 53.630 | 55.328 | 1.00 | 7.51 | DIC |
| ATOM | 174 | CA | ALA | 24 | 46.939 | 55.011 | 55.152 | 1.00 | 7.86 | DIC |
| ATOM | 175 | CB | ALA | 24 | 45.825 | 55.834 | 54.493 | 1.00 | 8.90 | DIC |
| ATOM | 176 | C | ALA | 24 | 47.367 | 55.664 | 56.465 | 1.00 | 8.07 | DIC |
| ATOM | 177 | O | ALA | 24 | 48.332 | 56.436 | 56.499 | 1.00 | 6.11 | DIC |
| ATOM | 178 | N | ALA | 25 | 46.654 | 55.353 | 57.543 | 1.00 | 7.25 | DIC |
| ATOM | 179 | CA | ALA | 25 | 46.961 | 55.923 | 58.853 | 1.00 | 7.99 | DIC |
| ATOM | 180 | CB | ALA | 25 | 45.705 | 55.908 | 59.736 | 1.00 | 6.76 | DIC |
| ATOM | 181 | C | ALA | 25 | 48.112 | 55.212 | 59.569 | 1.00 | 8.57 | DIC |
| ATOM | 182 | O | ALA | 25 | 48.504 | 55.611 | 60.663 | 1.00 | 7.27 | DIC |
| ATOM | 183 | N | HIS | 26 | 48.646 | 54.157 | 58.960 | 1.00 | 8.84 | DIC |
| ATOM | 184 | CA | HIS | 26 | 49.758 | 53.424 | 59.563 | 1.00 | 9.94 | DIC |
| ATOM | 185 | CB | HIS | 26 | 49.258 | 52.138 | 60.227 | 1.00 | 10.38 | DIC |
| ATOM | 186 | CG | HIS | 26 | 48.185 | 52.363 | 61.246 | 1.00 | 12.18 | DIC |
| ATOM | 187 | CD2 | HIS | 26 | 48.251 | 52.548 | 62.587 | 1.00 | 12.40 | DIC |
| ATOM | 188 | ND1 | HIS | 26 | 46.848 | 52.431 | 60.917 | 1.00 | 12.47 | DIC |
| ATOM | 189 | CE1 | HIS | 26 | 46.137 | 52.644 | 62.010 | 1.00 | 13.32 | DIC |
| ATOM | 190 | NE2 | HIS | 26 | 46.965 | 52.719 | 63.037 | 1.00 | 11.84 | DIC |
| ATOM | 191 | C | HIS | 26 | 50.804 | 53.090 | 58.502 | 1.00 | 8.30 | DIC |
| ATOM | 192 | O | HIS | 26 | 51.035 | 51.923 | 58.179 | 1.00 | 7.59 | DIC |
| ATOM | 193 | N | PRO | 27 | 51.465 | 54.124 | 57.959 | 1.00 | 8.95 | DIC |
| ATOM | 194 | CD | PRO | 27 | 51.399 | 55.521 | 58.424 | 1.00 | 9.38 | DIC |
| ATOM | 195 | CA | PRO | 27 | 52.490 | 53.970 | 56.927 | 1.00 | 9.59 | DIC |
| ATOM | 196 | CB | PRO | 27 | 52.940 | 55.408 | 56.672 | 1.00 | 10.14 | DIC |
| ATOM | 197 | CG | PRO | 27 | 52.738 | 56.064 | 57.999 | 1.00 | 11.05 | DIC |
| ATOM | 198 | C | PRO | 27 | 53.652 | 53.045 | 57.278 | 1.00 | 10.29 | DIC |
| ATOM | 199 | O | PRO | 27 | 54.227 | 52.414 | 56.389 | 1.00 | 12.94 | DIC |
| ATOM | 200 | N | ASP | 28 | 54.000 | 52.949 | 58.557 | 1.00 | 10.20 | DIC |
| ATOM | 201 | CA | ASP | 28 | 55.119 | 52.088 | 58.935 | 1.00 | 9.90 | DIC |
| ATOM | 202 | CB | ASP | 28 | 56.284 | 52.933 | 59.456 | 1.00 | 10.34 | DIC |
| ATOM | 203 | CG | ASP | 28 | 57.552 | 52.116 | 59.643 | 1.00 | 10.37 | DIC |
| ATOM | 204 | OD1 | ASP | 28 | 57.857 | 51.288 | 58.758 | 1.00 | 11.78 | DIC |
| ATOM | 205 | OD2 | ASP | 28 | 58.241 | 52.306 | 60.665 | 1.00 | 9.99 | DIC |
| ATOM | 206 | C | ASP | 28 | 54.765 | 51.009 | 59.951 | 1.00 | 11.28 | DIC |
| ATOM | 207 | O | ASP | 28 | 55.609 | 50.577 | 60.737 | 1.00 | 11.87 | DIC |
| ATOM | 208 | N | THR | 29 | 53.510 | 50.576 | 59.935 | 1.00 | 10.17 | DIC |
| ATOM | 209 | CA | THR | 29 | 53.062 | 49.523 | 60.836 | 1.00 | 11.26 | DIC |
| ATOM | 210 | CB | THR | 29 | 51.948 | 50.007 | 61.791 | 1.00 | 9.97 | DIC |
| ATOM | 211 | OG1 | THR | 29 | 52.424 | 51.115 | 62.566 | 1.00 | 10.16 | DIC |
| ATOM | 212 | CG2 | THR | 29 | 51.537 | 48.886 | 62.737 | 1.00 | 10.26 | DIC |
| ATOM | 213 | C | THR | 29 | 52.508 | 48.385 | 59.992 | 1.00 | 11.82 | DIC |
| ATOM | 214 | O | THR | 29 | 51.784 | 48.617 | 59.022 | 1.00 | 11.70 | DIC |
| ATOM | 215 | N | GLU | 30 | 52.868 | 47.159 | 60.349 | 1.00 | 11.89 | DIC |
| ATOM | 216 | CA | GLU | 30 | 52.382 | 45.987 | 59.632 | 1.00 | 14.04 | DIC |
| ATOM | 217 | CB | GLU | 30 | 53.142 | 44.743 | 60.097 | 1.00 | 17.64 | DIC |
| ATOM | 218 | CG | GLU | 30 | 53.297 | 43.655 | 59.051 | 1.00 | 25.15 | DIC |
| ATOM | 219 | CD | GLU | 30 | 54.060 | 44.126 | 57.822 | 1.00 | 27.98 | DIC |
| ATOM | 220 | OE1 | GLU | 30 | 53.418 | 44.612 | 56.869 | 1.00 | 28.55 | DIC |
| ATOM | 221 | OE2 | GLU | 30 | 55.306 | 44.026 | 57.816 | 1.00 | 31.94 | DIC |
| ATOM | 222 | C | GLU | 30 | 50.903 | 45.848 | 59.982 | 1.00 | 12.56 | DIC |
| ATOM | 223 | O | GLU | 30 | 50.545 | 45.798 | 61.158 | 1.00 | 13.85 | DIC |
| ATOM | 224 | N | ILE | 31 | 50.041 | 45.810 | 58.972 | 1.00 | 10.51 | DIC |
| ATOM | 225 | CA | ILE | 31 | 48.610 | 45.666 | 59.222 | 1.00 | 11.18 | DIC |
| ATOM | 226 | CB | ILE | 31 | 47.780 | 46.780 | 58.526 | 1.00 | 11.20 | DIC |
| ATOM | 227 | CG2 | ILE | 31 | 46.287 | 46.575 | 58.809 | 1.00 | 9.63 | DIC |
| ATOM | 228 | CG1 | ILE | 31 | 48.218 | 48.159 | 59.028 | 1.00 | 10.58 | DIC |
| ATOM | 229 | CD | ILE | 31 | 47.875 | 48.435 | 60.480 | 1.00 | 11.64 | DIC |
| ATOM | 230 | C | ILE | 31 | 48.142 | 44.317 | 58.696 | 1.00 | 10.56 | DIC |
| ATOM | 231 | O | ILE | 31 | 48.127 | 44.082 | 57.487 | 1.00 | 11.51 | DIC |
| ATOM | 232 | N | ARG | 32 | 47.770 | 43.430 | 59.609 | 1.00 | 9.50 | DIC |
| ATOM | 233 | CA | ARG | 32 | 47.296 | 42.108 | 59.227 | 1.00 | 9.26 | DIC |
| ATOM | 234 | CB | ARG | 32 | 47.919 | 41.042 | 60.137 | 1.00 | 12.86 | DIC |
| ATOM | 235 | CG | ARG | 32 | 49.451 | 41.101 | 60.168 | 1.00 | 15.87 | DIC |
| ATOM | 236 | CD | ARG | 32 | 50.090 | 39.888 | 60.831 | 1.00 | 19.22 | DIC |
| ATOM | 237 | NE | ARG | 32 | 49.697 | 39.723 | 62.228 | 1.00 | 23.57 | DIC |
| ATOM | 238 | CZ | ARG | 32 | 50.227 | 38.819 | 63.048 | 1.00 | 26.66 | DIC |
| ATOM | 239 | NH1 | ARG | 32 | 51.177 | 38.002 | 62.608 | 1.00 | 27.60 | DIC |
| ATOM | 240 | NH2 | ARG | 32 | 49.803 | 38.723 | 64.304 | 1.00 | 25.75 | DIC |
| ATOM | 241 | C | ARG | 32 | 45.774 | 42.083 | 59.328 | 1.00 | 8.78 | DIC |
| ATOM | 242 | O | ARG | 32 | 45.219 | 42.140 | 60.426 | 1.00 | 6.14 | DIC |
| ATOM | 243 | N | PHE | 33 | 45.111 | 42.021 | 58.174 | 1.00 | 7.42 | DIC |
| ATOM | 244 | CA | PHE | 33 | 43.653 | 41.993 | 58.107 | 1.00 | 6.88 | DIC |
| ATOM | 245 | CB | PHE | 33 | 43.159 | 42.646 | 56.809 | 1.00 | 6.79 | DIC |
| ATOM | 246 | CG | PHE | 33 | 43.424 | 44.126 | 56.715 | 1.00 | 6.63 | DIC |
| ATOM | 247 | CD1 | PHE | 33 | 42.732 | 45.027 | 57.521 | 1.00 | 6.30 | DIC |
| ATOM | 248 | CD2 | PHE | 33 | 44.333 | 44.622 | 55.784 | 1.00 | 6.14 | DIC |
| ATOM | 249 | CE1 | PHE | 33 | 42.936 | 46.403 | 57.399 | 1.00 | 6.64 | DIC |
| ATOM | 250 | CE2 | PHE | 33 | 44.548 | 45.995 | 55.651 | 1.00 | 7.13 | DIC |
| ATOM | 251 | CZ | PHE | 33 | 43.845 | 46.889 | 56.462 | 1.00 | 7.26 | DIC |
| ATOM | 252 | C | PHE | 33 | 43.083 | 40.575 | 58.175 | 1.00 | 7.51 | DIC |
| ATOM | 253 | O | PHE | 33 | 43.565 | 39.662 | 57.501 | 1.00 | 6.94 | DIC |
| ATOM | 254 | N | HIS | 34 | 42.048 | 40.413 | 58.992 | 1.00 | 6.28 | DIC |
| ATOM | 255 | CA | HIS | 34 | 41.345 | 39.141 | 59.159 | 1.00 | 7.93 | DIC |
| ATOM | 256 | CB | HIS | 34 | 41.450 | 38.652 | 60.604 | 1.00 | 8.52 | DIC |
| ATOM | 257 | CG | HIS | 34 | 42.855 | 38.517 | 61.100 | 1.00 | 10.09 | DIC |
| ATOM | 258 | CD2 | HIS | 34 | 43.703 | 39.428 | 61.632 | 1.00 | 11.33 | DIC |
| ATOM | 259 | ND1 | HIS | 34 | 43.541 | 37.322 | 61.079 | 1.00 | 11.33 | DIC |
| ATOM | 260 | CE1 | HIS | 34 | 44.751 | 37.502 | 61.578 | 1.00 | 12.87 | DIC |
| ATOM | 261 | NE2 | HIS | 34 | 44.874 | 38.772 | 61.921 | 1.00 | 11.31 | DIC |
| ATOM | 262 | C | HIS | 34 | 39.891 | 39.483 | 58.847 | 1.00 | 7.56 | DIC |
| ATOM | 263 | O | HIS | 34 | 39.260 | 40.244 | 59.579 | 1.00 | 6.31 | DIC |
| ATOM | 264 | N | VAL | 35 | 39.358 | 38.928 | 57.766 | 1.00 | 6.19 | DIC |
| ATOM | 265 | CA | VAL | 35 | 37.990 | 39.232 | 57.384 | 1.00 | 7.23 | DIC |
| ATOM | 266 | CB | VAL | 35 | 37.924 | 39.665 | 55.900 | 1.00 | 6.63 | DIC |
| ATOM | 267 | CG1 | VAL | 35 | 36.498 | 40.013 | 55.515 | 1.00 | 8.73 | DIC |
| ATOM | 268 | CG2 | VAL | 35 | 38.840 | 40.851 | 55.669 | 1.00 | 8.74 | DIC |
| ATOM | 269 | C | VAL | 35 | 37.013 | 38.081 | 57.596 | 1.00 | 8.74 | DIC |
| ATOM | 270 | O | VAL | 35 | 37.174 | 37.000 | 57.023 | 1.00 | 6.03 | DIC |
| ATOM | 271 | N | LEU | 36 | 36.010 | 38.314 | 58.438 | 1.00 | 7.38 | DIC |
| ATOM | 272 | CA | LEU | 36 | 34.984 | 37.307 | 58.683 | 1.00 | 8.45 | DIC |
| ATOM | 273 | CB | LEU | 36 | 34.190 | 37.639 | 59.954 | 1.00 | 7.92 | DIC |
| ATOM | 274 | CG | LEU | 36 | 34.993 | 37.773 | 61.259 | 1.00 | 8.01 | DIC |
| ATOM | 275 | CD1 | LEU | 36 | 34.031 | 37.960 | 62.427 | 1.00 | 7.60 | DIC |
| ATOM | 276 | CD2 | LEU | 36 | 35.847 | 36.535 | 61.492 | 1.00 | 7.27 | DIC |
| ATOM | 277 | C | LEU | 36 | 34.112 | 37.432 | 57.432 | 1.00 | 9.33 | DIC |
| ATOM | 278 | O | LEU | 36 | 33.319 | 38.365 | 57.299 | 1.00 | 8.93 | DIC |
| ATOM | 279 | N | ASP | 37 | 34.304 | 36.495 | 56.511 | 1.00 | 9.60 | DIC |
| ATOM | 280 | CA | ASP | 37 | 33.624 | 36.471 | 55.218 | 1.00 | 10.03 | DIC |
| ATOM | 281 | CB | ASP | 37 | 34.582 | 35.844 | 54.199 | 1.00 | 11.29 | DIC |
| ATOM | 282 | CG | ASP | 37 | 33.980 | 35.713 | 52.820 | 1.00 | 10.76 | DIC |
| ATOM | 283 | OD1 | ASP | 37 | 32.772 | 35.965 | 52.659 | 1.00 | 10.09 | DIC |
| ATOM | 284 | OD2 | ASP | 37 | 34.728 | 35.344 | 51.894 | 1.00 | 13.51 | DIC |
| ATOM | 285 | C | ASP | 37 | 32.287 | 35.728 | 55.223 | 1.00 | 11.39 | DIC |
| ATOM | 286 | O | ASP | 37 | 32.247 | 34.496 | 55.212 | 1.00 | 9.17 | DIC |
| ATOM | 287 | N | ALA | 38 | 31.196 | 36.489 | 55.209 | 1.00 | 11.46 | DIC |
| ATOM | 288 | CA | ALA | 38 | 29.853 | 35.917 | 55.230 | 1.00 | 13.65 | DIC |
| ATOM | 289 | CB | ALA | 38 | 28.914 | 36.832 | 56.015 | 1.00 | 14.69 | DIC |
| ATOM | 290 | C | ALA | 38 | 29.262 | 35.632 | 53.851 | 1.00 | 14.91 | DIC |
| ATOM | 291 | O | ALA | 38 | 28.045 | 35.490 | 53.714 | 1.00 | 17.68 | DIC |
| ATOM | 292 | N | GLY | 39 | 30.111 | 35.543 | 52.834 | 1.00 | 14.37 | DIC |
| ATOM | 293 | CA | GLY | 39 | 29.608 | 35.255 | 51.502 | 1.00 | 13.29 | DIC |
| ATOM | 294 | C | GLY | 39 | 30.109 | 36.181 | 50.410 | 1.00 | 12.56 | DIC |
| ATOM | 295 | O | GLY | 39 | 29.416 | 36.405 | 49.421 | 1.00 | 13.32 | DIC |
| ATOM | 296 | N | ILE | 40 | 31.308 | 36.724 | 50.588 | 1.00 | 10.63 | DIC |
| ATOM | 297 | CA | ILE | 40 | 31.906 | 37.616 | 49.597 | 1.00 | 9.24 | DIC |
| ATOM | 298 | CB | ILE | 40 | 33.142 | 38.337 | 50.192 | 1.00 | 8.46 | DIC |
| ATOM | 299 | CG2 | ILE | 40 | 33.741 | 39.293 | 49.168 | 1.00 | 7.79 | DIC |
| ATOM | 300 | CG1 | ILE | 40 | 32.729 | 39.099 | 51.456 | 1.00 | 6.53 | DIC |
| ATOM | 301 | CD | ILE | 40 | 33.894 | 39.641 | 52.273 | 1.00 | 9.79 | DIC |
| ATOM | 302 | C | ILE | 40 | 32.324 | 36.806 | 48.363 | 1.00 | 9.40 | DIC |
| ATOM | 303 | O | ILE | 40 | 32.951 | 35.753 | 48.482 | 1.00 | 8.50 | DIC |
| ATOM | 304 | N | SER | 41 | 31.972 | 37.294 | 47.179 | 1.00 | 10.73 | DIC |
| ATOM | 305 | CA | SER | 41 | 32.307 | 36.591 | 45.942 | 1.00 | 11.14 | DIC |
| ATOM | 306 | CB | SER | 41 | 31.669 | 37.295 | 44.742 | 1.00 | 11.01 | DIC |
| ATOM | 307 | OG | SER | 41 | 32.317 | 38.526 | 44.477 | 1.00 | 12.46 | DIC |
| ATOM | 308 | C | SER | 41 | 33.816 | 36.512 | 45.736 | 1.00 | 11.15 | DIC |
| ATOM | 309 | O | SER | 41 | 34.569 | 37.313 | 46.290 | 1.00 | 11.69 | DIC |
| ATOM | 310 | N | GLU | 42 | 34.253 | 35.541 | 44.940 | 1.00 | 12.21 | DIC |
| ATOM | 311 | CA | GLU | 42 | 35.673 | 35.371 | 44.659 | 1.00 | 11.82 | DIC |
| ATOM | 312 | CB | GLU | 42 | 35.906 | 34.153 | 43.755 | 1.00 | 15.43 | DIC |
| ATOM | 313 | CG | GLU | 42 | 37.376 | 33.842 | 43.483 | 1.00 | 19.50 | DIC |
| ATOM | 314 | CD | GLU | 42 | 37.989 | 34.710 | 42.392 | 1.00 | 24.05 | DIC |
| ATOM | 315 | OE1 | GLU | 42 | 39.235 | 34.789 | 42.331 | 1.00 | 27.65 | DIC |
| ATOM | 316 | OE2 | GLU | 42 | 37.235 | 35.302 | 41.589 | 1.00 | 24.25 | DIC |
| ATOM | 317 | C | GLU | 42 | 36.199 | 36.625 | 43.973 | 1.00 | 10.77 | DIC |
| ATOM | 318 | O | GLU | 42 | 37.309 | 37.070 | 44.248 | 1.00 | 9.02 | DIC |
| ATOM | 319 | N | ALA | 43 | 35.389 | 37.193 | 43.086 | 1.00 | 10.24 | DIC |
| ATOM | 320 | CA | ALA | 43 | 35.776 | 38.395 | 42.359 | 1.00 | 11.39 | DIC |
| ATOM | 321 | CB | ALA | 43 | 34.720 | 38.744 | 41.307 | 1.00 | 10.87 | DIC |
| ATOM | 322 | C | ALA | 43 | 35.963 | 39.564 | 43.317 | 1.00 | 10.68 | DIC |
| ATOM | 323 | O | ALA | 43 | 36.906 | 40.343 | 43.181 | 1.00 | 9.26 | DIC |
| ATOM | 324 | N | ASN | 44 | 35.070 | 39.681 | 44.294 | 1.00 | 11.27 | DIC |
| ATOM | 325 | CA | ASN | 44 | 35.169 | 40.774 | 45.253 | 1.00 | 11.66 | DIC |
| ATOM | 326 | CB | ASN | 44 | 33.839 | 40.954 | 45.997 | 1.00 | 10.82 | DIC |
| ATOM | 327 | CG | ASN | 44 | 32.789 | 41.645 | 45.141 | 1.00 | 13.91 | DIC |
| ATOM | 328 | OD1 | ASN | 44 | 33.094 | 42.603 | 44.429 | 1.00 | 14.16 | DIC |
| ATOM | 329 | ND2 | ASN | 44 | 31.547 | 41.171 | 45.213 | 1.00 | 13.53 | DIC |
| ATOM | 330 | C | ASN | 44 | 36.326 | 40.602 | 46.235 | 1.00 | 12.23 | DIC |
| ATOM | 331 | O | ASN | 44 | 36.912 | 41.585 | 46.686 | 1.00 | 12.30 | DIC |
| ATOM | 332 | N | ARG | 45 | 36.662 | 39.358 | 46.566 | 1.00 | 12.83 | DIC |
| ATOM | 333 | CA | ARG | 45 | 37.775 | 39.117 | 47.477 | 1.00 | 13.27 | DIC |
| ATOM | 334 | CB | ARG | 45 | 37.838 | 37.640 | 47.888 | 1.00 | 14.62 | DIC |
| ATOM | 335 | CG | ARG | 45 | 36.519 | 37.103 | 48.420 | 1.00 | 19.99 | DIC |
| ATOM | 336 | CD | ARG | 45 | 36.679 | 36.267 | 49.680 | 1.00 | 23.85 | DIC |
| ATOM | 337 | NE | ARG | 45 | 37.610 | 35.153 | 49.520 | 1.00 | 27.49 | DIC |
| ATOM | 338 | CZ | ARG | 45 | 37.746 | 34.167 | 50.404 | 1.00 | 30.19 | DIC |
| ATOM | 339 | NH1 | ARG | 45 | 37.007 | 34.152 | 51.506 | 1.00 | 31.72 | DIC |
| ATOM | 340 | NH2 | ARG | 45 | 38.634 | 33.202 | 50.199 | 1.00 | 29.60 | DIC |
| ATOM | 341 | C | ARG | 45 | 39.063 | 39.516 | 46.760 | 1.00 | 11.59 | DIC |
| ATOM | 342 | O | ARG | 45 | 39.936 | 40.159 | 47.337 | 1.00 | 8.60 | DIC |
| ATOM | 343 | N | ALA | 46 | 39.167 | 39.141 | 45.490 | 1.00 | 10.34 | DIC |
| ATOM | 344 | CA | ALA | 46 | 40.347 | 39.475 | 44.704 | 1.00 | 10.59 | DIC |
| ATOM | 345 | CB | ALA | 46 | 40.257 | 38.830 | 43.321 | 1.00 | 11.03 | DIC |
| ATOM | 346 | C | ALA | 46 | 40.480 | 40.987 | 44.567 | 1.00 | 10.58 | DIC |
| ATOM | 347 | O | ALA | 46 | 41.576 | 41.535 | 44.686 | 1.00 | 11.52 | DIC |
| ATOM | 348 | N | AL A | 47 | 39.360 | 41.661 | 44.318 | 1.00 | 10.04 | DIC |
| ATOM | 349 | CA | ALA | 47 | 39.363 | 43.112 | 44.155 | 1.00 | 9.25 | DIC |
| ATOM | 350 | CB | ALA | 47 | 37.997 | 43.587 | 43.672 | 1.00 | 11.48 | DIC |
| ATOM | 351 | C | ALA | 47 | 39.752 | 43.844 | 45.443 | 1.00 | 9.33 | DIC |
| ATOM | 352 | O | ALA | 47 | 40.459 | 44.849 | 45.399 | 1.00 | 8.11 | DIC |
| ATOM | 353 | N | VAL | 48 | 39.284 | 43.353 | 46.587 | 1.00 | 9.19 | DIC |
| ATOM | 354 | CA | VAL | 48 | 39.624 | 43.982 | 47.858 | 1.00 | 9.94 | DIC |
| ATOM | 355 | CB | VAL | 48 | 38.902 | 43.302 | 49.049 | 1.00 | 9.44 | DIC |
| ATOM | 356 | CG1 | VAL | 48 | 39.431 | 43.863 | 50.367 | 1.00 | 7.91 | DIC |
| ATOM | 357 | CG2 | VAL | 48 | 37.403 | 43.535 | 48.955 | 1.00 | 6.88 | DIC |
| ATOM | 358 | C | VAL | 48 | 41.133 | 43.867 | 48.067 | 1.00 | 10.57 | DIC |
| ATOM | 359 | O | VAL | 48 | 41.808 | 44.851 | 48.359 | 1.00 | 10.43 | DIC |
| ATOM | 360 | N | ALA | 49 | 41.655 | 42.657 | 47.898 | 1.00 | 11.00 | DIC |
| ATOM | 361 | CA | ALA | 49 | 43.079 | 42.401 | 48.075 | 1.00 | 12.61 | DIC |
| ATOM | 362 | CB | ALA | 49 | 43.352 | 40.903 | 47.955 | 1.00 | 11.98 | DIC |
| ATOM | 363 | C | ALA | 49 | 43.951 | 43.170 | 47.085 | 1.00 | 13.08 | DIC |
| ATOM | 364 | O | ALA | 49 | 45.011 | 43.683 | 47.446 | 1.00 | 13.68 | DIC |
| ATOM | 365 | N | ALA | 50 | 43.502 | 43.251 | 45.838 | 1.00 | 14.66 | DIC |
| ATOM | 366 | CA | ALA | 50 | 44.256 | 43.948 | 44.801 | 1.00 | 14.50 | DIC |
| ATOM | 367 | CB | ALA | 50 | 43.530 | 43.828 | 43.463 | 1.00 | 15.47 | DIC |
| ATOM | 368 | C | ALA | 50 | 44.497 | 45.421 | 45.132 | 1.00 | 15.39 | DIC |
| ATOM | 369 | O | ALA | 50 | 45.490 | 46.004 | 44.698 | 1.00 | 16.30 | DIC |
| ATOM | 370 | N | ASN | 51 | 43.590 | 46.020 | 45.899 | 1.00 | 14.62 | DIC |
| ATOM | 371 | CA | ASN | 51 | 43.708 | 47.429 | 46.269 | 1.00 | 14.05 | DIC |
| ATOM | 372 | CB | ASN | 51 | 42.317 | 48.048 | 46.439 | 1.00 | 12.38 | DIC |
| ATOM | 373 | CG | ASN | 51 | 41.634 | 48.316 | 45.116 | 1.00 | 12.84 | DIC |
| ATOM | 374 | OD1 | ASN | 51 | 42.110 | 49.114 | 44.314 | 1.00 | 13.23 | DIC |
| ATOM | 375 | ND2 | ASN | 51 | 40.511 | 47.651 | 44.881 | 1.00 | 11.33 | DIC |
| ATOM | 376 | C | ASN | 51 | 44.512 | 47.668 | 47.542 | 1.00 | 15.83 | DIC |
| ATOM | 377 | O | ASN | 51 | 44.891 | 48.804 | 47.841 | 1.00 | 17.23 | DIC |
| ATOM | 378 | N | LEU | 52 | 44.773 | 46.607 | 48.295 | 1.00 | 15.52 | DIC |
| ATOM | 379 | CA | LEU | 52 | 45.520 | 46.754 | 49.535 | 1.00 | 17.67 | DIC |
| ATOM | 380 | CB | LEU | 52 | 45.106 | 45.669 | 50.530 | 1.00 | 15.40 | DIC |
| ATOM | 381 | CG | LEU | 52 | 43.604 | 45.719 | 50.825 | 1.00 | 16.03 | DIC |
| ATOM | 382 | CD1 | LEU | 52 | 43.233 | 44.666 | 51.852 | 1.00 | 16.15 | DIC |
| ATOM | 383 | CD2 | LEU | 52 | 43.232 | 47.106 | 51.322 | 1.00 | 15.81 | DIC |
| ATOM | 384 | C | LEU | 52 | 47.021 | 46.730 | 49.305 | 1.00 | 19.77 | DIC |
| ATOM | 385 | O | LEU | 52 | 47.497 | 46.237 | 48.277 | 1.00 | 18.50 | DIC |
| ATOM | 386 | N | ARG | 53 | 47.754 | 47.275 | 50.271 | 1.00 | 21.96 | DIC |
| ATOM | 387 | CA | ARG | 53 | 49.207 | 47.361 | 50.210 | 1.00 | 25.17 | DIC |
| ATOM | 388 | CB | ARG | 53 | 49.773 | 47.572 | 51.619 | 1.00 | 24.25 | DIC |
| ATOM | 389 | CG | ARG | 53 | 50.746 | 48.726 | 51.703 | 1.00 | 23.54 | DIC |
| ATOM | 390 | CD | ARG | 53 | 50.512 | 49.603 | 52.931 | 1.00 | 20.57 | DIC |
| ATOM | 391 | NE | ARG | 53 | 50.702 | 48.882 | 54.184 | 1.00 | 16.68 | DIC |
| ATOM | 392 | CZ | ARG | 53 | 50.784 | 49.472 | 55.373 | 1.00 | 17.69 | DIC |
| ATOM | 393 | NH1 | ARG | 53 | 50.694 | 50.794 | 55.469 | 1.00 | 14.96 | DIC |
| ATOM | 394 | NH2 | ARG | 53 | 50.957 | 48.744 | 56.467 | 1.00 | 16.12 | DIC |
| ATOM | 395 | C | ARG | 53 | 49.811 | 46.124 | 49.565 | 1.00 | 26.76 | DIC |
| ATOM | 396 | O | ARG | 53 | 50.790 | 46.214 | 48.825 | 1.00 | 28.81 | DIC |
| ATOM | 397 | N | GLY | 55 | 49.212 | 44.969 | 49.834 | 1.00 | 28.95 | DIC |
| ATOM | 398 | CA | GLY | 55 | 49.702 | 43.742 | 49.245 | 1.00 | 30.90 | DIC |
| ATOM | 399 | C | GLY | 55 | 48.600 | 42.749 | 48.919 | 1.00 | 32.46 | DIC |
| ATOM | 400 | O | GLY | 55 | 48.361 | 42.412 | 47.756 | 1.00 | 33.41 | DIC |
| ATOM | 401 | N | GLY | 56 | 47.909 | 42.297 | 49.958 | 1.00 | 33.24 | DIC |
| ATOM | 402 | CA | GLY | 56 | 46.866 | 41.306 | 49.792 | 1.00 | 31.87 | DIC |
| ATOM | 403 | C | GLY | 56 | 47.421 | 40.103 | 50.521 | 1.00 | 31.64 | DIC |
| ATOM | 404 | O | GLY | 56 | 46.693 | 39.202 | 50.943 | 1.00 | 32.69 | DIC |
| ATOM | 405 | N | GLY | 57 | 48.743 | 40.105 | 50.666 | 1.00 | 29.97 | DIC |
| ATOM | 406 | CA | GLY | 57 | 49.415 | 39.035 | 51.373 | 1.00 | 28.09 | DIC |
| ATOM | 407 | C | GLY | 57 | 49.162 | 39.249 | 52.849 | 1.00 | 26.72 | DIC |
| ATOM | 408 | O | GLY | 57 | 49.474 | 38.394 | 53.677 | 1.00 | 28.17 | DIC |
| ATOM | 409 | N | ASN | 58 | 48.592 | 40.409 | 53.171 | 1.00 | 24.96 | DIC |
| ATOM | 410 | CA | ASN | 58 | 48.272 | 40.755 | 54.550 | 1.00 | 22.29 | DIC |
| ATOM | 411 | CB | ASN | 58 | 48.850 | 42.121 | 54.919 | 1.00 | 24.14 | DIC |
| ATOM | 412 | CG | ASN | 58 | 50.242 | 42.023 | 55.510 | 1.00 | 27.73 | DIC |
| ATOM | 413 | OD1 | ASN | 58 | 50.457 | 41.332 | 56.509 | 1.00 | 27.14 | DIC |
| ATOM | 414 | ND2 | ASN | 58 | 51.198 | 42.717 | 54.898 | 1.00 | 28.37 | DIC |
| ATOM | 415 | C | ASN | 58 | 46.777 | 40.748 | 54.832 | 1.00 | 19.33 | DIC |
| ATOM | 416 | O | ASN | 58 | 46.315 | 41.409 | 55.761 | 1.00 | 17.00 | DIC |
| ATOM | 417 | N | ILE | 59 | 46.016 | 40.020 | 54.021 | 1.00 | 16.23 | DIC |
| ATOM | 418 | CA | ILE | 59 | 44.584 | 39.920 | 54.254 | 1.00 | 14.14 | DIC |
| ATOM | 419 | CB | ILE | 59 | 43.766 | 40.863 | 53.329 | 1.00 | 15.68 | DIC |
| ATOM | 420 | CG2 | ILE | 59 | 43.980 | 40.502 | 51.862 | 1.00 | 14.29 | DIC |
| ATOM | 421 | CG1 | ILE | 59 | 42.283 | 40.774 | 53.699 | 1.00 | 14.17 | DIC |
| ATOM | 422 | CD | ILE | 59 | 41.413 | 41.795 | 52.996 | 1.00 | 16.56 | DIC |
| ATOM | 423 | C | ILE | 59 | 44.133 | 38.477 | 54.071 | 1.00 | 13.72 | DIC |
| ATOM | 424 | O | ILE | 59 | 44.418 | 37.839 | 53.056 | 1.00 | 12.08 | DIC |
| ATOM | 425 | N | ARG | 60 | 43.456 | 37.953 | 55.084 | 1.00 | 13.05 | DIC |
| ATOM | 426 | CA | ARG | 60 | 42.968 | 36.586 | 55.031 | 1.00 | 12.60 | DIC |
| ATOM | 427 | CB | ARG | 60 | 43.685 | 35.730 | 56.082 | 1.00 | 15.76 | DIC |
| ATOM | 428 | CG | ARG | 60 | 43.217 | 34.284 | 56.154 | 1.00 | 20.30 | DIC |
| ATOM | 429 | CD | ARG | 60 | 44.272 | 33.351 | 56.769 | 1.00 | 24.60 | DIC |
| ATOM | 430 | NE | ARG | 60 | 44.682 | 33.733 | 58.118 | 1.00 | 29.24 | DIC |
| ATOM | 431 | CZ | ARG | 60 | 45.606 | 34.650 | 58.398 | 1.00 | 31.92 | DIC |
| ATOM | 432 | NH1 | ARG | 60 | 46.232 | 35.291 | 57.419 | 1.00 | 34.11 | DIC |
| ATOM | 433 | NH2 | ARG | 60 | 45.908 | 34.926 | 59.660 | 1.00 | 32.11 | DIC |
| ATOM | 434 | C | ARG | 60 | 41.469 | 36.596 | 55.274 | 1.00 | 13.28 | DIC |
| ATOM | 435 | O | ARG | 60 | 40.986 | 37.219 | 56.225 | 1.00 | 12.52 | DIC |
| ATOM | 436 | N | PHE | 61 | 40.728 | 35.932 | 54.393 | 1.00 | 9.98 | DIC |
| ATOM | 437 | CA | PHE | 61 | 39.283 | 35.868 | 54.543 | 1.00 | 11.03 | DIC |
| ATOM | 438 | CB | PHE | 61 | 38.593 | 35.909 | 53.180 | 1.00 | 10.11 | DIC |
| ATOM | 439 | CG | PHE | 61 | 38.864 | 37.165 | 52.404 | 1.00 | 8.69 | DIC |
| ATOM | 440 | CD1 | PHE | 61 | 40.001 | 37.279 | 51.611 | 1.00 | 10.62 | DIC |
| ATOM | 441 | CD2 | PHE | 61 | 37.984 | 38.239 | 52.470 | 1.00 | 8.62 | DIC |
| ATOM | 442 | CE1 | PHE | 61 | 40.255 | 38.444 | 50.896 | 1.00 | 10.15 | DIC |
| ATOM | 443 | CE2 | PHE | 61 | 38.231 | 39.411 | 51.757 | 1.00 | 10.17 | DIC |
| ATOM | 444 | CZ | PHE | 61 | 39.368 | 39.511 | 50.969 | 1.00 | 8.33 | DIC |
| ATOM | 445 | C | PHE | 61 | 38.949 | 34.578 | 55.265 | 1.00 | 10.30 | DIC |
| ATOM | 446 | O | PHE | 61 | 39.474 | 33.520 | 54.934 | 1.00 | 10.02 | DIC |
| ATOM | 447 | N | ILE | 62 | 38.080 | 34.678 | 56.262 | 1.00 | 10.75 | DIC |
| ATOM | 448 | CA | ILE | 62 | 37.682 | 33.525 | 57.053 | 1.00 | 10.78 | DIC |
| ATOM | 449 | CB | ILE | 62 | 37.949 | 33.791 | 58.550 | 1.00 | 11.91 | DIC |
| ATOM | 450 | CG2 | ILE | 62 | 37.750 | 32.516 | 59.358 | 1.00 | 9.27 | DIC |
| ATOM | 451 | CG1 | ILE | 62 | 39.380 | 34.301 | 58.728 | 1.00 | 11.15 | DIC |
| ATOM | 452 | CD | ILE | 62 | 39.672 | 34.864 | 60.108 | 1.00 | 13.99 | DIC |
| ATOM | 453 | C | ILE | 62 | 36.199 | 33.259 | 56.829 | 1.00 | 11.22 | DIC |
| ATOM | 454 | O | ILE | 62 | 35.352 | 34.083 | 57.172 | 1.00 | 8.84 | DIC |
| ATOM | 455 | N | ASP | 63 | 35.891 | 32.102 | 56.249 | 1.00 | 12.77 | DIC |
| ATOM | 456 | CA | ASP | 63 | 34.509 | 31.743 | 55.963 | 1.00 | 15.03 | DIC |
| ATOM | 457 | CB | ASP | 63 | 34.450 | 30.425 | 55.186 | 1.00 | 19.05 | DIC |
| ATOM | 458 | CG | ASP | 63 | 35.078 | 30.533 | 53.815 | 1.00 | 22.78 | DIC |
| ATOM | 459 | OD1 | ASP | 63 | 34.633 | 31.392 | 53.027 | 1.00 | 24.85 | DIC |
| ATOM | 460 | OD2 | ASP | 63 | 36.017 | 29.762 | 53.526 | 1.00 | 26.44 | DIC |
| ATOM | 461 | C | ASP | 63 | 33.638 | 31.624 | 57.201 | 1.00 | 15.28 | DIC |
| ATOM | 462 | O | ASP | 63 | 33.989 | 30.943 | 58.164 | 1.00 | 13.87 | DIC |
| ATOM | 463 | N | VAL | 64 | 32.499 | 32.301 | 57.170 | 1.00 | 14.42 | DIC |
| ATOM | 464 | CA | VAL | 64 | 31.558 | 32.237 | 58.274 | 1.00 | 14.74 | DIC |
| ATOM | 465 | CB | VAL | 64 | 31.449 | 33.587 | 59.025 | 1.00 | 15.80 | DIC |
| ATOM | 466 | CG1 | VAL | 64 | 32.778 | 33.930 | 59.669 | 1.00 | 16.58 | DIC |
| ATOM | 467 | CG2 | VAL | 64 | 31.025 | 34.684 | 58.073 | 1.00 | 16.06 | DIC |
| ATOM | 468 | C | VAL | 64 | 30.196 | 31.865 | 57.706 | 1.00 | 14.22 | DIC |
| ATOM | 469 | O | VAL | 64 | 29.826 | 32.308 | 56.617 | 1.00 | 14.84 | DIC |
| ATOM | 470 | N | ASN | 65 | 29.463 | 31.033 | 58.435 | 1.00 | 12.47 | DIC |
| ATOM | 471 | CA | ASN | 65 | 28.137 | 30.614 | 58.010 | 1.00 | 13.60 | DIC |
| ATOM | 472 | CB | ASN | 65 | 27.857 | 29.182 | 58.476 | 1.00 | 14.16 | DIC |
| ATOM | 473 | CG | ASN | 65 | 26.564 | 28.624 | 57.906 | 1.00 | 16.61 | DIC |
| ATOM | 474 | OD1 | ASN | 65 | 25.625 | 29.367 | 57.620 | 1.00 | 16.00 | DIC |
| ATOM | 475 | ND2 | ASN | 65 | 26.505 | 27.305 | 57.752 | 1.00 | 14.91 | DIC |
| ATOM | 476 | C | ASN | 65 | 27.123 | 31.560 | 58.647 | 1.00 | 14.85 | DIC |
| ATOM | 477 | O | ASN | 65 | 26.895 | 31.510 | 59.856 | 1.00 | 15.54 | DIC |
| ATOM | 478 | N | PRO | 66 | 26.509 | 32.443 | 57.846 | 1.00 | 15.26 | DIC |
| ATOM | 479 | CD | PRO | 66 | 26.691 | 32.671 | 56.401 | 1.00 | 15.57 | DIC |
| ATOM | 480 | CA | PRO | 66 | 25.526 | 33.376 | 58.404 | 1.00 | 15.89 | DIC |
| ATOM | 481 | CB | PRO | 66 | 25.166 | 34.256 | 57.208 | 1.00 | 17.28 | DIC |
| ATOM | 482 | CG | PRO | 66 | 25.406 | 33.358 | 56.029 | 1.00 | 16.80 | DIC |
| ATOM | 483 | C | PRO | 66 | 24.311 | 32.678 | 59.019 | 1.00 | 15.69 | DIC |
| ATOM | 484 | O | PRO | 66 | 23.646 | 33.231 | 59.893 | 1.00 | 16.02 | DIC |
| ATOM | 485 | N | ALA | 67 | 24.028 | 31.461 | 58.568 | 1.00 | 14.83 | DIC |
| ATOM | 486 | CA | ALA | 67 | 22.896 | 30.713 | 59.101 | 1.00 | 15.30 | DIC |
| ATOM | 487 | CB | ALA | 67 | 22.749 | 29.382 | 58.366 | 1.00 | 16.72 | DIC |
| ATOM | 488 | C | ALA | 67 | 23.082 | 30.466 | 60.597 | 1.00 | 15.63 | DIC |
| ATOM | 489 | O | ALA | 67 | 22.112 | 30.252 | 61.322 | 1.00 | 15.80 | DIC |
| ATOM | 490 | N | ASP | 68 | 24.330 | 30.505 | 61.056 | 1.00 | 14.70 | DIC |
| ATOM | 491 | CA | ASP | 68 | 24.634 | 30.282 | 62.466 | 1.00 | 15.13 | DIC |
| ATOM | 492 | CB | ASP | 68 | 26.133 | 30.478 | 62.736 | 1.00 | 15.76 | DIC |
| ATOM | 493 | CG | ASP | 68 | 26.987 | 29.337 | 62.201 | 1.00 | 17.55 | DIC |
| ATOM | 494 | OD1 | ASP | 68 | 28.216 | 29.367 | 62.418 | 1.00 | 17.24 | DIC |
| ATOM | 495 | OD2 | ASP | 68 | 26.439 | 28.415 | 61.570 | 1.00 | 18.46 | DIC |
| ATOM | 496 | C | ASP | 68 | 23.854 | 31.213 | 63.391 | 1.00 | 14.63 | DIC |
| ATOM | 497 | O | ASP | 68 | 23.503 | 30.838 | 64.507 | 1.00 | 15.13 | DIC |
| ATOM | 498 | N | PHE | 69 | 23.580 | 32.424 | 62.923 | 1.00 | 13.83 | DIC |
| ATOM | 499 | CA | PHE | 69 | 22.880 | 33.406 | 63.740 | 1.00 | 14.98 | DIC |
| ATOM | 500 | CB | PHE | 69 | 23.691 | 34.708 | 63.745 | 1.00 | 13.87 | DIC |
| ATOM | 501 | CG | PHE | 69 | 25.180 | 34.484 | 63.681 | 1.00 | 14.17 | DIC |
| ATOM | 502 | CD1 | PHE | 69 | 25.823 | 34.341 | 62.452 | 1.00 | 12.43 | DIC |
| ATOM | 503 | CD2 | PHE | 69 | 25.929 | 34.355 | 64.847 | 1.00 | 12.05 | DIC |
| ATOM | 504 | CE1 | PHE | 69 | 27.192 | 34.071 | 62.385 | 1.00 | 13.43 | DIC |
| ATOM | 505 | CE2 | PHE | 69 | 27.297 | 34.084 | 64.794 | 1.00 | 12.77 | DIC |
| ATOM | 506 | CZ | PHE | 69 | 27.932 | 33.941 | 63.561 | 1.00 | 11.69 | DIC |
| ATOM | 507 | C | PHE | 69 | 21.447 | 33.672 | 63.285 | 1.00 | 15.85 | DIC |
| ATOM | 508 | O | PHE | 69 | 20.877 | 34.719 | 63.587 | 1.00 | 14.86 | DIC |
| ATOM | 509 | N | ALA | 70 | 20.863 | 32.708 | 62.585 | 1.00 | 16.36 | DIC |
| ATOM | 510 | CA | ALA | 70 | 19.502 | 32.840 | 62.074 | 1.00 | 19.02 | DIC |
| ATOM | 511 | CB | ALA | 70 | 19.135 | 31.594 | 61.269 | 1.00 | 20.70 | DIC |
| ATOM | 512 | C | ALA | 70 | 18.437 | 33.095 | 63.143 | 1.00 | 19.98 | DIC |
| ATOM | 513 | O | ALA | 70 | 17.366 | 33.616 | 62.840 | 1.00 | 22.15 | DIC |
| ATOM | 514 | N | GLY | 71 | 18.723 | 32.734 | 64.388 | 1.00 | 18.98 | DIC |
| ATOM | 515 | CA | GLY | 71 | 17.740 | 32.935 | 65.437 | 1.00 | 19.23 | DIC |
| ATOM | 516 | C | GLY | 71 | 17.753 | 34.294 | 66.114 | 1.00 | 19.37 | DIC |
| ATOM | 517 | O | GLY | 71 | 16.795 | 34.653 | 66.806 | 1.00 | 18.55 | DIC |
| ATOM | 518 | N | PHE | 72 | 18.824 | 35.056 | 65.910 | 1.00 | 17.12 | DIC |
| ATOM | 519 | CA | PHE | 72 | 18.959 | 36.370 | 66.528 | 1.00 | 16.52 | DIC |
| ATOM | 520 | CB | PHE | 72 | 20.444 | 36.736 | 66.627 | 1.00 | 17.36 | DIC |
| ATOM | 521 | CG | PHE | 72 | 21.244 | 35.800 | 67.502 | 1.00 | 20.69 | DIC |
| ATOM | 522 | CD1 | PHE | 72 | 22.622 | 35.942 | 67.615 | 1.00 | 20.90 | DIC |
| ATOM | 523 | CD2 | PHE | 72 | 20.616 | 34.781 | 68.220 | 1.00 | 20.65 | DIC |
| ATOM | 524 | CE1 | PHE | 72 | 23.365 | 35.084 | 68.429 | 1.00 | 22.32 | DIC |
| ATOM | 525 | CE2 | PHE | 72 | 21.350 | 33.918 | 69.036 | 1.00 | 21.81 | DIC |
| ATOM | 526 | CZ | PHE | 72 | 22.728 | 34.071 | 69.139 | 1.00 | 22.14 | DIC |
| ATOM | 527 | C | PHE | 72 | 18.182 | 37.476 | 65.809 | 1.00 | 15.69 | DIC |
| ATOM | 528 | O | PHE | 72 | 18.062 | 37.475 | 64.582 | 1.00 | 16.74 | DIC |
| ATOM | 529 | N | PRO | 73 | 17.644 | 38.438 | 66.576 | 1.00 | 14.19 | DIC |
| ATOM | 530 | CD | PRO | 73 | 17.784 | 38.566 | 68.037 | 1.00 | 14.66 | DIC |
| ATOM | 531 | CA | PRO | 73 | 16.870 | 39.558 | 66.033 | 1.00 | 13.58 | DIC |
| ATOM | 532 | CB | PRO | 73 | 16.430 | 40.313 | 67.287 | 1.00 | 14.13 | DIC |
| ATOM | 533 | CG | PRO | 73 | 17.522 | 40.037 | 68.251 | 1.00 | 14.64 | DIC |
| ATOM | 534 | C | PRO | 73 | 17.632 | 40.441 | 65.053 | 1.00 | 13.06 | DIC |
| ATOM | 535 | O | PRO | 73 | 18.839 | 40.647 | 65.183 | 1.00 | 11.47 | DIC |
| ATOM | 536 | N | LEU | 74 | 16.912 | 40.951 | 64.061 | 1.00 | 12.77 | DIC |
| ATOM | 537 | CA | LEU | 74 | 17.498 | 41.819 | 63.049 | 1.00 | 14.73 | DIC |
| ATOM | 538 | CB | LEU | 74 | 17.807 | 40.997 | 61.793 | 1.00 | 16.34 | DIC |
| ATOM | 539 | CG | LEU | 74 | 18.987 | 41.439 | 60.926 | 1.00 | 18.53 | DIC |
| ATOM | 540 | CD1 | LEU | 74 | 20.262 | 41.429 | 61.767 | 1.00 | 15.81 | DIC |
| ATOM | 541 | CD2 | LEU | 74 | 19.127 | 40.503 | 59.727 | 1.00 | 17.94 | DIC |
| ATOM | 542 | C | LEU | 74 | 16.493 | 42.935 | 62.731 | 1.00 | 14.70 | DIC |
| ATOM | 543 | O | LEU | 74 | 16.013 | 43.048 | 61.603 | 1.00 | 17.35 | DIC |
| ATOM | 544 | N | ASN | 75 | 16.186 | 43.758 | 63.732 | 1.00 | 12.80 | DIC |
| ATOM | 545 | CA | ASN | 75 | 15.222 | 44.848 | 63.573 | 1.00 | 11.77 | DIC |
| ATOM | 546 | CB | ASN | 75 | 14.595 | 45.205 | 64.934 | 1.00 | 13.05 | DIC |
| ATOM | 547 | CG | ASN | 75 | 15.634 | 45.441 | 66.031 | 1.00 | 15.54 | DIC |
| ATOM | 548 | OD1 | ASN | 75 | 16.359 | 44.524 | 66.427 | 1.00 | 14.39 | DIC |
| ATOM | 549 | ND2 | ASN | 75 | 15.699 | 46.673 | 66.533 | 1.00 | 13.85 | DIC |
| ATOM | 550 | C | ASN | 75 | 15.741 | 46.122 | 62.900 | 1.00 | 11.45 | DIC |
| ATOM | 551 | O | ASN | 75 | 14.947 | 46.933 | 62.421 | 1.00 | 10.31 | DIC |
| ATOM | 552 | N | ILE | 76 | 17.059 | 46.302 | 62.865 | 1.00 | 9.67 | DIC |
| ATOM | 553 | CA | ILE | 76 | 17.649 | 47.486 | 62.242 | 1.00 | 8.64 | DIC |
| ATOM | 554 | CB | ILE | 76 | 18.989 | 47.859 | 62.920 | 1.00 | 10.45 | DIC |
| ATOM | 555 | CG2 | ILE | 76 | 19.526 | 49.164 | 62.336 | 1.00 | 10.96 | DIC |
| ATOM | 556 | CG1 | ILE | 76 | 18.771 | 48.010 | 64.433 | 1.00 | 11.13 | DIC |
| ATOM | 557 | CD | ILE | 76 | 20.026 | 48.383 | 65.224 | 1.00 | 10.33 | DIC |
| ATOM | 558 | C | ILE | 76 | 17.858 | 47.180 | 60.761 | 1.00 | 6.66 | DIC |
| ATOM | 559 | O | ILE | 76 | 18.744 | 46.417 | 60.386 | 1.00 | 6.82 | DIC |
| ATOM | 560 | N | ARG | 77 | 17.022 | 47.791 | 59.929 | 1.00 | 6.55 | DIC |
| ATOM | 561 | CA | ARG | 77 | 17.020 | 47.565 | 58.487 | 1.00 | 4.60 | DIC |
| ATOM | 562 | CB | ARG | 77 | 16.009 | 48.506 | 57.830 | 1.00 | 6.27 | DIC |
| ATOM | 563 | CG | ARG | 77 | 15.689 | 48.175 | 56.373 | 1.00 | 7.44 | DIC |
| ATOM | 564 | CD | ARG | 77 | 14.783 | 49.243 | 55.776 | 1.00 | 13.42 | DIC |
| ATOM | 565 | NE | ARG | 77 | 14.442 | 48.985 | 54.379 | 1.00 | 15.86 | DIC |
| ATOM | 566 | CZ | ARG | 77 | 14.048 | 49.925 | 53.525 | 1.00 | 17.42 | DIC |
| ATOM | 567 | NH1 | ARG | 77 | 13.948 | 51.184 | 53.927 | 1.00 | 18.20 | DIC |
| ATOM | 568 | NH2 | ARG | 77 | 13.755 | 49.611 | 52.270 | 1.00 | 19.12 | DIC |
| ATOM | 569 | C | ARG | 77 | 18.341 | 47.637 | 57.727 | 1.00 | 5.81 | DIC |
| ATOM | 570 | O | ARG | 77 | 18.598 | 46.795 | 56.870 | 1.00 | 5.17 | DIC |
| ATOM | 571 | N | HIS | 78 | 19.182 | 48.623 | 58.022 | 1.00 | 6.00 | DIC |
| ATOM | 572 | CA | HIS | 78 | 20.440 | 48.744 | 57.287 | 1.00 | 9.02 | DIC |
| ATOM | 573 | CB | HIS | 78 | 20.979 | 50.175 | 57.389 | 1.00 | 10.15 | DIC |
| ATOM | 574 | CG | HIS | 78 | 21.474 | 50.544 | 58.751 | 1.00 | 9.33 | DIC |
| ATOM | 575 | CD2 | HIS | 78 | 20.848 | 51.131 | 59.797 | 1.00 | 6.75 | DIC |
| ATOM | 576 | ND1 | HIS | 78 | 22.765 | 50.293 | 59.168 | 1.00 | 11.81 | DIC |
| ATOM | 577 | CE1 | HIS | 78 | 22.911 | 50.712 | 60.412 | 1.00 | 8.70 | DIC |
| ATOM | 578 | NE2 | HIS | 78 | 21.762 | 51.224 | 60.817 | 1.00 | 11.84 | DIC |
| ATOM | 579 | C | HIS | 78 | 21.509 | 47.751 | 57.734 | 1.00 | 8.20 | DIC |
| ATOM | 580 | O | HIS | 78 | 22.624 | 47.761 | 57.219 | 1.00 | 7.53 | DIC |
| ATOM | 581 | N | ILE | 79 | 21.162 | 46.872 | 58.669 | 1.00 | 7.37 | DIC |
| ATOM | 582 | CA | ILE | 79 | 22.129 | 45.895 | 59.158 | 1.00 | 7.68 | DIC |
| ATOM | 583 | CB | ILE | 79 | 22.262 | 45.979 | 60.687 | 1.00 | 5.65 | DIC |
| ATOM | 584 | CG2 | ILE | 79 | 23.256 | 44.932 | 61.180 | 1.00 | 4.12 | DIC |
| ATOM | 585 | CG1 | ILE | 79 | 22.715 | 47.384 | 61.083 | 1.00 | 4.91 | DIC |
| ATOM | 586 | CD | ILE | 79 | 22.696 | 47.634 | 62.575 | 1.00 | 5.56 | DIC |
| ATOM | 587 | C | ILE | 79 | 21.794 | 44.460 | 58.783 | 1.00 | 8.61 | DIC |
| ATOM | 588 | O | ILE | 79 | 20.693 | 43.983 | 59.046 | 1.00 | 9.77 | DIC |
| ATOM | 589 | N | SER | 80 | 22.752 | 43.773 | 58.169 | 1.00 | 9.76 | DIC |
| ATOM | 590 | CA | SER | 80 | 22.561 | 42.379 | 57.774 | 1.00 | 8.74 | DIC |
| ATOM | 591 | CB | SER | 80 | 23.402 | 42.050 | 56.541 | 1.00 | 8.44 | DIC |
| ATOM | 592 | OG | SER | 80 | 24.781 | 42.091 | 56.852 | 1.00 | 8.55 | DIC |
| ATOM | 593 | C | SER | 80 | 22.973 | 41.462 | 58.927 | 1.00 | 9.00 | DIC |
| ATOM | 594 | O | SER | 80 | 23.580 | 41.913 | 59.903 | 1.00 | 8.52 | DIC |
| ATOM | 595 | N | ILE | 81 | 22.637 | 40.181 | 58.800 | 1.00 | 8.69 | DIC |
| ATOM | 596 | CA | ILE | 81 | 22.932 | 39.167 | 59.814 | 1.00 | 9.32 | DIC |
| ATOM | 597 | CB | ILE | 81 | 22.342 | 37.793 | 59.391 | 1.00 | 11.01 | DIC |
| ATOM | 598 | CG2 | ILE | 81 | 23.127 | 37.231 | 58.211 | 1.00 | 12.45 | DIC |
| ATOM | 599 | CG1 | ILE | 81 | 22.387 | 36.802 | 60.557 | 1.00 | 13.43 | DIC |
| ATOM | 600 | CD | ILE | 81 | 21.500 | 37.182 | 61.726 | 1.00 | 16.17 | DIC |
| ATOM | 601 | C | ILE | 81 | 24.433 | 39.007 | 60.082 | 1.00 | 8.61 | DIC |
| ATOM | 602 | O | ILE | 81 | 24.831 | 38.496 | 61.123 | 1.00 | 9.44 | DIC |
| ATOM | 603 | N | THR | 82 | 25.259 | 39.450 | 59.144 | 1.00 | 8.29 | DIC |
| ATOM | 604 | CA | THR | 82 | 26.711 | 39.346 | 59.290 | 1.00 | 9.90 | DIC |
| ATOM | 605 | CB | THR | 82 | 27.416 | 39.899 | 58.032 | 1.00 | 9.68 | DIC |
| ATOM | 606 | OG1 | THR | 82 | 27.081 | 39.078 | 56.908 | 1.00 | 11.57 | DIC |
| ATOM | 607 | CG2 | THR | 82 | 28.924 | 39.902 | 58.208 | 1.00 | 11.14 | DIC |
| ATOM | 608 | C | THR | 82 | 27.226 | 40.083 | 60.532 | 1.00 | 9.31 | DIC |
| ATOM | 609 | O | THR | 82 | 28.308 | 39.784 | 61.044 | 1.00 | 11.54 | DIC |
| ATOM | 610 | N | THR | 83 | 26.442 | 41.044 | 61.007 | 1.00 | 7.10 | DIC |
| ATOM | 611 | CA | THR | 83 | 26.793 | 41.843 | 62.178 | 1.00 | 6.02 | DIC |
| ATOM | 612 | CB | THR | 83 | 25.681 | 42.891 | 62.460 | 1.00 | 4.53 | DIC |
| ATOM | 613 | OG1 | THR | 83 | 26.153 | 43.855 | 63.407 | 1.00 | 3.63 | DIC |
| ATOM | 614 | CG2 | THR | 83 | 24.428 | 42.217 | 63.004 | 1.00 | 2.50 | DIC |
| ATOM | 615 | C | THR | 83 | 27.028 | 40.992 | 63.439 | 1.00 | 5.43 | DIC |
| ATOM | 616 | O | THR | 83 | 27.686 | 41.435 | 64.389 | 1.00 | 2.82 | DIC |
| ATOM | 617 | N | TYR | 84 | 26.490 | 39.775 | 63.442 | 1.00 | 4.36 | DIC |
| ATOM | 618 | CA | TYR | 84 | 26.636 | 38.865 | 64.579 | 1.00 | 4.81 | DIC |
| ATOM | 619 | CB | TYR | 84 | 25.399 | 37.962 | 64.707 | 1.00 | 5.98 | DIC |
| ATOM | 620 | CG | TYR | 84 | 24.158 | 38.632 | 65.252 | 1.00 | 5.88 | DIC |
| ATOM | 621 | CD1 | TYR | 84 | 23.083 | 38.948 | 64.415 | 1.00 | 7.00 | DIC |
| ATOM | 622 | CE1 | TYR | 84 | 21.922 | 39.534 | 64.921 | 1.00 | 7.41 | DIC |
| ATOM | 623 | CD2 | TYR | 84 | 24.040 | 38.920 | 66.611 | 1.00 | 6.51 | DIC |
| ATOM | 624 | CE2 | TYR | 84 | 22.882 | 39.503 | 67.129 | 1.00 | 6.56 | DIC |
| ATOM | 625 | CZ | TYR | 84 | 21.829 | 39.805 | 66.277 | 1.00 | 7.16 | DIC |
| ATOM | 626 | OH | TYR | 84 | 20.682 | 40.370 | 66.782 | 1.00 | 7.35 | DIC |
| ATOM | 627 | C | TYR | 84 | 27.872 | 37.966 | 64.507 | 1.00 | 5.01 | DIC |
| ATOM | 628 | O | TYR | 84 | 28.228 | 37.317 | 65.497 | 1.00 | 6.35 | DIC |
| ATOM | 629 | N | ALA | 85 | 28.524 | 37.923 | 63.350 | 1.00 | 4.75 | DIC |
| ATOM | 630 | CA | ALA | 85 | 29.697 | 37.068 | 63.174 | 1.00 | 5.04 | DIC |
| ATOM | 631 | CB | ALA | 85 | 30.266 | 37.240 | 61.765 | 1.00 | 5.81 | DIC |
| ATOM | 632 | C | ALA | 85 | 30.788 | 37.323 | 64.211 | 1.00 | 4.45 | DIC |
| ATOM | 633 | O | ALA | 85 | 31.506 | 36.409 | 64.601 | 1.00 | 3.97 | DIC |
| ATOM | 634 | N | ARG | 86 | 30.899 | 38.566 | 64.663 | 1.00 | 5.28 | DIC |
| ATOM | 635 | CA | ARG | 86 | 31.918 | 38.932 | 65.636 | 1.00 | 6.46 | DIC |
| ATOM | 636 | CB | ARG | 86 | 31.830 | 40.436 | 65.927 | 1.00 | 6.54 | DIC |
| ATOM | 637 | CG | ARG | 86 | 30.578 | 40.885 | 66.662 | 1.00 | 7.60 | DIC |
| ATOM | 638 | CD | ARG | 86 | 30.479 | 42.411 | 66.665 | 1.00 | 6.22 | DIC |
| ATOM | 639 | NE | ARG | 86 | 29.753 | 42.918 | 65.502 | 1.00 | 7.60 | DIC |
| ATOM | 640 | CZ | ARG | 86 | 29.592 | 44.209 | 65.223 | 1.00 | 8.07 | DIC |
| ATOM | 641 | NH1 | ARG | 86 | 30.121 | 45.135 | 66.016 | 1.00 | 6.32 | DIC |
| ATOM | 642 | NH2 | ARG | 86 | 28.866 | 44.578 | 64.173 | 1.00 | 6.28 | DIC |
| ATOM | 643 | C | ARG | 86 | 31.834 | 38.123 | 66.933 | 1.00 | 6.26 | DIC |
| ATOM | 644 | O | ARG | 86 | 32.825 | 37.970 | 67.642 | 1.00 | 5.75 | DIC |
| ATOM | 645 | N | LEU | 87 | 30.654 | 37.594 | 67.231 | 1.00 | 6.67 | DIC |
| ATOM | 646 | CA | LEU | 87 | 30.453 | 36.800 | 68.444 | 1.00 | 9.15 | DIC |
| ATOM | 647 | CB | LEU | 87 | 28.978 | 36.423 | 68.579 | 1.00 | 9.79 | DIC |
| ATOM | 648 | CG | LEU | 87 | 27.968 | 37.573 | 68.662 | 1.00 | 10.81 | DIC |
| ATOM | 649 | CD1 | LEU | 87 | 26.565 | 36.998 | 68.625 | 1.00 | 10.16 | DIC |
| ATOM | 650 | CD2 | LEU | 87 | 28.186 | 38.385 | 69.928 | 1.00 | 9.69 | DIC |
| ATOM | 651 | C | LEU | 87 | 31.309 | 35.531 | 68.500 | 1.00 | 8.69 | DIC |
| ATOM | 652 | O | LEU | 87 | 31.663 | 35.069 | 69.583 | 1.00 | 8.91 | DIC |
| ATOM | 653 | N | LYS | 88 | 31.633 | 34.964 | 67.337 | 1.00 | 9.25 | DIC |
| ATOM | 654 | CA | LYS | 88 | 32.453 | 33.749 | 67.269 | 1.00 | 7.47 | DIC |
| ATOM | 655 | CB | LYS | 88 | 31.882 | 32.787 | 66.215 | 1.00 | 8.55 | DIC |
| ATOM | 656 | CG | LYS | 88 | 30.591 | 32.084 | 66.618 | 1.00 | 5.05 | DIC |
| ATOM | 657 | CD | LYS | 88 | 30.239 | 31.000 | 65.605 | 1.00 | 9.57 | DIC |
| ATOM | 658 | CE | LYS | 88 | 29.127 | 30.091 | 66.111 | 1.00 | 11.92 | DIC |
| ATOM | 659 | NZ | LYS | 88 | 28.966 | 28.889 | 65.234 | 1.00 | 9.27 | DIC |
| ATOM | 660 | C | LYS | 88 | 33.921 | 34.034 | 66.932 | 1.00 | 8.56 | DIC |
| ATOM | 661 | O | LYS | 88 | 34.635 | 33.166 | 66.426 | 1.00 | 7.98 | DIC |
| ATOM | 662 | N | LEU | 89 | 34.374 | 35.247 | 67.221 | 1.00 | 8.21 | DIC |
| ATOM | 663 | CA | LEU | 89 | 35.746 | 35.638 | 66.924 | 1.00 | 9.63 | DIC |
| ATOM | 664 | CB | LEU | 89 | 35.968 | 37.079 | 67.392 | 1.00 | 11.64 | DIC |
| ATOM | 665 | CG | LEU | 89 | 36.996 | 37.961 | 66.683 | 1.00 | 15.58 | DIC |
| ATOM | 666 | CD1 | LEU | 89 | 36.916 | 37.802 | 65.176 | 1.00 | 14.23 | DIC |
| ATOM | 667 | CD2 | LEU | 89 | 36.731 | 39.405 | 67.074 | 1.00 | 15.43 | DIC |
| ATOM | 668 | C | LEU | 89 | 36.760 | 34.688 | 67.564 | 1.00 | 10.10 | DIC |
| ATOM | 669 | O | LEU | 89 | 37.825 | 34.423 | 66.998 | 1.00 | 10.90 | DIC |
| ATOM | 670 | N | GLY | 90 | 36.420 | 34.165 | 68.738 | 1.00 | 8.62 | DIC |
| ATOM | 671 | CA | GLY | 90 | 37.306 | 33.238 | 69.422 | 1.00 | 9.71 | DIC |
| ATOM | 672 | C | GLY | 90 | 37.439 | 31.920 | 68.679 | 1.00 | 10.61 | DIC |
| ATOM | 673 | O | GLY | 90 | 38.417 | 31.195 | 68.858 | 1.00 | 9.41 | DIC |
| ATOM | 674 | N | GLU | 91 | 36.447 | 31.607 | 67.848 | 1.00 | 9.92 | DIC |
| ATOM | 675 | CA | GLU | 91 | 36.453 | 30.379 | 67.058 | 1.00 | 11.25 | DIC |
| ATOM | 676 | CB | GLU | 91 | 35.022 | 29.875 | 66.839 | 1.00 | 14.13 | DIC |
| ATOM | 677 | CG | GLU | 91 | 34.281 | 29.413 | 68.084 | 1.00 | 17.36 | DIC |
| ATOM | 678 | CD | GLU | 91 | 32.897 | 28.871 | 67.750 | 1.00 | 20.17 | DIC |
| ATOM | 679 | OE1 | GLU | 91 | 32.791 | 28.072 | 66.794 | 1.00 | 19.66 | DIC |
| ATOM | 680 | OE2 | GLU | 91 | 31.921 | 29.234 | 68.441 | 1.00 | 19.51 | DIC |
| ATOM | 681 | C | GLU | 91 | 37.093 | 30.594 | 65.683 | 1.00 | 11.62 | DIC |
| ATOM | 682 | O | GLU | 91 | 37.663 | 29.664 | 65.100 | 1.00 | 11.92 | DIC |
| ATOM | 683 | N | TYR | 92 | 36.989 | 31.819 | 65.173 | 1.00 | 10.48 | DIC |
| ATOM | 684 | CA | TYR | 92 | 37.516 | 32.165 | 63.854 | 1.00 | 10.15 | DIC |
| ATOM | 685 | CB | TYR | 92 | 36.725 | 33.331 | 63.253 | 1.00 | 9.65 | DIC |
| ATOM | 686 | CG | TYR | 92 | 35.244 | 33.078 | 63.086 | 1.00 | 11.40 | DIC |
| ATOM | 687 | CD1 | TYR | 92 | 34.774 | 31.872 | 62.568 | 1.00 | 10.51 | DIC |
| ATOM | 688 | CE1 | TYR | 92 | 33.411 | 31.655 | 62.371 | 1.00 | 13.36 | DIC |
| ATOM | 689 | CD2 | TYR | 92 | 34.312 | 34.066 | 63.406 | 1.00 | 11.86 | DIC |
| ATOM | 690 | CE2 | TYR | 92 | 32.948 | 33.861 | 63.211 | 1.00 | 13.22 | DIC |
| ATOM | 691 | CZ | TYR | 92 | 32.505 | 32.654 | 62.695 | 1.00 | 14.23 | DIC |
| ATOM | 692 | OH | TYR | 92 | 31.159 | 32.447 | 62.507 | 1.00 | 12.62 | DIC |
| ATOM | 693 | C | TYR | 92 | 38.992 | 32.518 | 63.776 | 1.00 | 10.04 | DIC |
| ATOM | 694 | O | TYR | 92 | 39.601 | 32.361 | 62.723 | 1.00 | 9.17 | DIC |
| ATOM | 695 | N | ILE | 93 | 39.563 | 33.014 | 64.870 | 1.00 | 11.91 | DIC |
| ATOM | 696 | CA | ILE | 93 | 40.971 | 33.405 | 64.886 | 1.00 | 13.98 | DIC |
| ATOM | 697 | CB | ILE | 93 | 41.122 | 34.938 | 65.044 | 1.00 | 15.92 | DIC |
| ATOM | 698 | CG2 | ILE | 93 | 42.597 | 35.318 | 65.098 | 1.00 | 14.58 | DIC |
| ATOM | 699 | CG1 | ILE | 93 | 40.441 | 35.650 | 63.872 | 1.00 | 16.29 | DIC |
| ATOM | 700 | CD | ILE | 93 | 40.462 | 37.154 | 63.985 | 1.00 | 14.93 | DIC |
| ATOM | 701 | C | ILE | 93 | 41.728 | 32.721 | 66.018 | 1.00 | 15.16 | DIC |
| ATOM | 702 | O | ILE | 93 | 41.350 | 32.830 | 67.185 | 1.00 | 14.37 | DIC |
| ATOM | 703 | N | ALA | 94 | 42.804 | 32.028 | 65.663 | 1.00 | 14.83 | DIC |
| ATOM | 704 | CA | ALA | 94 | 43.608 | 31.305 | 66.639 | 1.00 | 19.11 | DIC |
| ATOM | 705 | CB | ALA | 94 | 43.703 | 29.845 | 66.231 | 1.00 | 19.20 | DIC |
| ATOM | 706 | C | ALA | 94 | 45.013 | 31.871 | 66.836 | 1.00 | 19.59 | DIC |
| ATOM | 707 | O | ALA | 94 | 45.592 | 31.747 | 67.915 | 1.00 | 20.84 | DIC |
| ATOM | 708 | N | ASP | 95 | 45.552 | 32.493 | 65.794 | 1.00 | 21.87 | DIC |
| ATOM | 709 | CA | ASP | 95 | 46.905 | 33.044 | 65.831 | 1.00 | 23.46 | DIC |
| ATOM | 710 | CB | ASP | 95 | 47.409 | 33.251 | 64.397 | 1.00 | 26.41 | DIC |
| ATOM | 711 | CG | ASP | 95 | 46.424 | 34.024 | 63.537 | 1.00 | 29.30 | DIC |
| ATOM | 712 | OD1 | ASP | 95 | 46.717 | 34.258 | 62.344 | 1.00 | 32.19 | DIC |
| ATOM | 713 | OD2 | ASP | 95 | 45.351 | 34.398 | 64.050 | 1.00 | 33.42 | DIC |
| ATOM | 714 | C | ASP | 95 | 47.115 | 34.332 | 66.626 | 1.00 | 22.72 | DIC |
| ATOM | 715 | O | ASP | 95 | 48.257 | 34.737 | 66.851 | 1.00 | 24.34 | DIC |
| ATOM | 716 | N | CYS | 96 | 46.036 | 34.968 | 67.069 | 1.00 | 21.07 | DIC |
| ATOM | 717 | CA | CYS | 96 | 46.168 | 36.221 | 67.811 | 1.00 | 19.00 | DIC |
| ATOM | 718 | CB | CYS | 96 | 45.485 | 37.356 | 67.043 | 1.00 | 17.51 | DIC |
| ATOM | 719 | SG | CYS | 96 | 46.000 | 37.495 | 65.337 | 1.00 | 17.81 | DIC |
| ATOM | 720 | C | CYS | 96 | 45.603 | 36.192 | 69.223 | 1.00 | 17.30 | DIC |
| ATOM | 721 | O | CYS | 96 | 44.532 | 35.636 | 69.459 | 1.00 | 18.98 | DIC |
| ATOM | 722 | N | ASP | 97 | 46.326 | 36.804 | 70.155 | 1.00 | 15.20 | DIC |
| ATOM | 723 | CA | ASP | 97 | 45.879 | 36.886 | 71.540 | 1.00 | 15.33 | DIC |
| ATOM | 724 | CB | ASP | 97 | 47.070 | 36.957 | 72.495 | 1.00 | 16.39 | DIC |
| ATOM | 725 | CG | ASP | 97 | 47.696 | 35.605 | 72.741 | 1.00 | 17.76 | DIC |
| ATOM | 726 | OD1 | ASP | 97 | 48.786 | 35.559 | 73.347 | 1.00 | 22.14 | DIC |
| ATOM | 727 | OD2 | ASP | 97 | 47.091 | 34.590 | 72.335 | 1.00 | 18.91 | DIC |
| ATOM | 728 | C | ASP | 97 | 45.041 | 38.146 | 71.689 | 1.00 | 14.04 | DIC |
| ATOM | 729 | O | ASP | 97 | 44.289 | 38.297 | 72.652 | 1.00 | 13.55 | DIC |
| ATOM | 730 | N | ALA | 98 | 45.192 | 39.051 | 70.728 | 1.00 | 12.61 | DIC |
| ATOM | 731 | CA | ALA | 98 | 44.457 | 40.309 | 70.727 | 1.00 | 12.75 | DIC |
| ATOM | 732 | CB | ALA | 98 | 45.220 | 41.362 | 71.513 | 1.00 | 14.90 | DIC |
| ATOM | 733 | C | ALA | 98 | 44.243 | 40.786 | 69.299 | 1.00 | 10.95 | DIC |
| ATOM | 734 | O | ALA | 98 | 45.130 | 40.664 | 68.456 | 1.00 | 10.19 | DIC |
| ATOM | 735 | N | VAL | 99 | 43.055 | 41.317 | 69.032 | 1.00 | 9.01 | DIC |
| ATOM | 736 | CA | VAL | 99 | 42.726 | 41.827 | 67.710 | 1.00 | 8.36 | DIC |
| ATOM | 737 | CB | VAL | 99 | 41.911 | 40.801 | 66.883 | 1.00 | 7.71 | DIC |
| ATOM | 738 | CG1 | VAL | 99 | 42.761 | 39.579 | 66.590 | 1.00 | 4.15 | DIC |
| ATOM | 739 | CG2 | VAL | 99 | 40.639 | 40.407 | 67.637 | 1.00 | 5.20 | DIC |
| ATOM | 740 | C | VAL | 99 | 41.902 | 43.090 | 67.862 | 1.00 | 8.06 | DIC |
| ATOM | 741 | O | VAL | 99 | 41.233 | 43.282 | 68.877 | 1.00 | 8.30 | DIC |
| ATOM | 742 | N | LEU | 100 | 41.965 | 43.961 | 66.864 | 1.00 | 6.36 | DIC |
| ATOM | 743 | CA | LEU | 100 | 41.193 | 45.193 | 66.905 | 1.00 | 5.80 | DIC |
| ATOM | 744 | CB | LEU | 100 | 42.084 | 46.407 | 66.611 | 1.00 | 5.45 | DIC |
| ATOM | 745 | CG | LEU | 100 | 41.418 | 47.790 | 66.524 | 1.00 | 8.26 | DIC |
| ATOM | 746 | CD1 | LEU | 100 | 40.403 | 47.975 | 67.641 | 1.00 | 6.61 | DIC |
| ATOM | 747 | CD2 | LEU | 100 | 42.492 | 48.867 | 66.602 | 1.00 | 8.45 | DIC |
| ATOM | 748 | C | LEU | 100 | 40.092 | 45.086 | 65.867 | 1.00 | 5.59 | DIC |
| ATOM | 749 | O | LEU | 100 | 40.352 | 45.145 | 64.663 | 1.00 | 5.35 | DIC |
| ATOM | 750 | N | TYR | 101 | 38.864 | 44.910 | 66.343 | 1.00 | 4.71 | DIC |
| ATOM | 751 | CA | TYR | 101 | 37.720 | 44.795 | 65.462 | 1.00 | 5.45 | DIC |
| ATOM | 752 | CB | TYR | 101 | 36.624 | 43.930 | 66.105 | 1.00 | 7.22 | DIC |
| ATOM | 753 | CG | TYR | 101 | 35.306 | 44.030 | 65.366 | 1.00 | 6.26 | DIC |
| ATOM | 754 | CD1 | TYR | 101 | 34.459 | 45.119 | 65.565 | 1.00 | 10.55 | DIC |
| ATOM | 755 | CE1 | TYR | 101 | 33.308 | 45.290 | 64.805 | 1.00 | 11.37 | DIC |
| ATOM | 756 | CD2 | TYR | 101 | 34.957 | 43.099 | 64.390 | 1.00 | 9.90 | DIC |
| ATOM | 757 | CE2 | TYR | 101 | 33.799 | 43.261 | 63.622 | 1.00 | 10.47 | DIC |
| ATOM | 758 | CZ | TYR | 101 | 32.987 | 44.361 | 63.834 | 1.00 | 10.64 | DIC |
| ATOM | 759 | OH | TYR | 101 | 31.868 | 44.565 | 63.051 | 1.00 | 16.19 | DIC |
| ATOM | 760 | C | TYR | 101 | 37.142 | 46.160 | 65.101 | 1.00 | 5.61 | DIC |
| ATOM | 761 | O | TYR | 101 | 36.947 | 47.020 | 65.968 | 1.00 | 4.16 | DIC |
| ATOM | 762 | N | LEU | 102 | 36.857 | 46.339 | 63.816 | 1.00 | 4.05 | DIC |
| ATOM | 763 | CA | LEU | 102 | 36.279 | 47.580 | 63.314 | 1.00 | 4.38 | DIC |
| ATOM | 764 | CB | LEU | 102 | 37.295 | 48.338 | 62.462 | 1.00 | 4.30 | DIC |
| ATOM | 765 | CG | LEU | 102 | 38.591 | 48.754 | 63.147 | 1.00 | 7.19 | DIC |
| ATOM | 766 | CD1 | LEU | 102 | 39.510 | 49.400 | 62.128 | 1.00 | 6.72 | DIC |
| ATOM | 767 | CD2 | LEU | 102 | 38.276 | 49.714 | 64.293 | 1.00 | 3.87 | DIC |
| ATOM | 768 | C | LEU | 102 | 35.061 | 47.286 | 62.451 | 1.00 | 4.47 | DIC |
| ATOM | 769 | O | LEU | 102 | 35.034 | 46.284 | 61.733 | 1.00 | 4.61 | DIC |
| ATOM | 770 | N | ASP | 103 | 34.054 | 48.153 | 62.534 | 1.00 | 3.95 | DIC |
| ATOM | 771 | CA | ASP | 103 | 32.860 | 48.016 | 61.715 | 1.00 | 4.24 | DIC |
| ATOM | 772 | CB | ASP | 103 | 31.761 | 48.979 | 62.177 | 1.00 | 2.65 | DIC |
| ATOM | 773 | CG | ASP | 103 | 30.737 | 48.327 | 63.095 | 1.00 | 2.68 | DIC |
| ATOM | 774 | OD1 | ASP | 103 | 29.754 | 49.012 | 63.436 | 1.00 | 4.76 | DIC |
| ATOM | 775 | OD2 | ASP | 103 | 30.897 | 47.152 | 63.482 | 1.00 | 3.70 | DIC |
| ATOM | 776 | C | ASP | 103 | 33.328 | 48.428 | 60.320 | 1.00 | 5.01 | DIC |
| ATOM | 777 | O | ASP | 103 | 34.417 | 48.975 | 60.169 | 1.00 | 3.63 | DIC |
| ATOM | 778 | N | ILE | 104 | 32.507 | 48.184 | 59.310 | 1.00 | 6.60 | DIC |
| ATOM | 779 | CA | ILE | 104 | 32.867 | 48.531 | 57.935 | 1.00 | 5.92 | DIC |
| ATOM | 780 | CB | ILE | 104 | 32.014 | 47.723 | 56.933 | 1.00 | 6.60 | DIC |
| ATOM | 781 | CG2 | ILE | 104 | 32.269 | 48.202 | 55.505 | 1.00 | 5.47 | DIC |
| ATOM | 782 | CG1 | ILE | 104 | 32.308 | 46.229 | 57.092 | 1.00 | 6.21 | DIC |
| ATOM | 783 | CD | ILE | 104 | 33.753 | 45.837 | 56.829 | 1.00 | 6.76 | DIC |
| ATOM | 784 | C | ILE | 104 | 32.675 | 50.018 | 57.655 | 1.00 | 6.14 | DIC |
| ATOM | 785 | O | ILE | 104 | 33.386 | 50.610 | 56.838 | 1.00 | 6.24 | DIC |
| ATOM | 786 | N | ASP | 105 | 31.712 | 50.618 | 58.344 | 1.00 | 4.96 | DIC |
| ATOM | 787 | CA | ASP | 105 | 31.399 | 52.028 | 58.167 | 1.00 | 5.98 | DIC |
| ATOM | 788 | CB | ASP | 105 | 29.930 | 52.274 | 58.513 | 1.00 | 4.82 | DIC |
| ATOM | 789 | CG | ASP | 105 | 29.609 | 51.925 | 59.956 | 1.00 | 3.16 | DIC |
| ATOM | 790 | OD1 | ASP | 105 | 30.337 | 51.092 | 60.532 | 1.00 | 2.79 | DIC |
| ATOM | 791 | OD2 | ASP | 105 | 28.627 | 52.466 | 60.507 | 1.00 | 5.38 | DIC |
| ATOM | 792 | C | ASP | 105 | 32.286 | 52.943 | 59.009 | 1.00 | 6.62 | DIC |
| ATOM | 793 | O | ASP | 105 | 31.790 | 53.838 | 59.693 | 1.00 | 6.81 | DIC |
| ATOM | 794 | N | VAL | 106 | 33.594 | 52.710 | 58.966 | 1.00 | 7.03 | DIC |
| ATOM | 795 | CA | VAL | 106 | 34.536 | 53.540 | 59.706 | 1.00 | 7.28 | DIC |
| ATOM | 796 | CB | VAL | 106 | 35.300 | 52.752 | 60.801 | 1.00 | 8.49 | DIC |
| ATOM | 797 | CG1 | VAL | 106 | 34.321 | 52.057 | 61.732 | 1.00 | 7.70 | DIC |
| ATOM | 798 | CG2 | VAL | 106 | 36.257 | 51.754 | 60.156 | 1.00 | 8.17 | DIC |
| ATOM | 799 | C | VAL | 106 | 35.575 | 54.126 | 58.761 | 1.00 | 6.79 | DIC |
| ATOM | 800 | O | VAL | 106 | 35.848 | 53.586 | 57.684 | 1.00 | 5.38 | DIC |
| ATOM | 801 | N | LEU | 107 | 36.151 | 55.243 | 59.180 | 1.00 | 7.44 | DIC |
| ATOM | 802 | CA | LEU | 107 | 37.179 | 55.916 | 58.410 | 1.00 | 9.27 | DIC |
| ATOM | 803 | CB | LEU | 107 | 36.598 | 57.173 | 57.756 | 1.00 | 11.52 | DIC |
| ATOM | 804 | CG | LEU | 107 | 36.654 | 57.330 | 56.232 | 1.00 | 15.69 | DIC |
| ATOM | 805 | CD1 | LEU | 107 | 36.132 | 56.088 | 55.542 | 1.00 | 17.09 | DIC |
| ATOM | 806 | CD2 | LEU | 107 | 35.829 | 58.543 | 55.834 | 1.00 | 16.31 | DIC |
| ATOM | 807 | C | LEU | 107 | 38.239 | 56.284 | 59.441 | 1.00 | 8.22 | DIC |
| ATOM | 808 | O | LEU | 107 | 38.038 | 57.190 | 60.241 | 1.00 | 8.43 | DIC |
| ATOM | 809 | N | VAL | 108 | 39.346 | 55.548 | 59.447 | 1.00 | 9.38 | DIC |
| ATOM | 810 | CA | VAL | 108 | 40.429 | 55.812 | 60.390 | 1.00 | 9.80 | DIC |
| ATOM | 811 | CB | VAL | 108 | 41.415 | 54.627 | 60.456 | 1.00 | 8.32 | DIC |
| ATOM | 812 | CG1 | VAL | 108 | 42.534 | 54.927 | 61.462 | 1.00 | 7.62 | DIC |
| ATOM | 813 | CG2 | VAL | 108 | 40.667 | 53.368 | 60.857 | 1.00 | 8.96 | DIC |
| ATOM | 814 | C | VAL | 108 | 41.165 | 57.056 | 59.926 | 1.00 | 11.09 | DIC |
| ATOM | 815 | O | VAL | 108 | 41.661 | 57.100 | 58.801 | 1.00 | 11.19 | DIC |
| ATOM | 816 | N | ARG | 109 | 41.234 | 58.061 | 60.794 | 1.00 | 11.01 | DIC |
| ATOM | 817 | CA | ARG | 109 | 41.879 | 59.327 | 60.453 | 1.00 | 12.38 | DIC |
| ATOM | 818 | CB | ARG | 109 | 40.927 | 60.487 | 60.743 | 1.00 | 14.98 | DIC |
| ATOM | 819 | CG | ARG | 109 | 39.453 | 60.116 | 60.700 | 1.00 | 19.75 | DIC |
| ATOM | 820 | CD | ARG | 109 | 38.650 | 61.137 | 59.922 | 1.00 | 23.34 | DIC |
| ATOM | 821 | NE | ARG | 109 | 39.023 | 62.512 | 60.241 | 1.00 | 26.83 | DIC |
| ATOM | 822 | CZ | ARG | 109 | 38.634 | 63.568 | 59.529 | 1.00 | 28.43 | DIC |
| ATOM | 823 | NH1 | ARG | 109 | 39.017 | 64.789 | 59.879 | 1.00 | 28.45 | DIC |
| ATOM | 824 | NH2 | ARG | 109 | 37.862 | 63.400 | 58.462 | 1.00 | 27.61 | DIC |
| ATOM | 825 | C | ARG | 109 | 43.197 | 59.582 | 61.181 | 1.00 | 12.26 | DIC |
| ATOM | 826 | O | ARG | 109 | 43.930 | 60.511 | 60.836 | 1.00 | 12.24 | DIC |
| ATOM | 827 | N | ASP | 110 | 43.484 | 58.780 | 62.201 | 1.00 | 10.65 | DIC |
| ATOM | 828 | CA | ASP | 110 | 44.723 | 58.934 | 62.954 | 1.00 | 10.25 | DIC |
| ATOM | 829 | CB | ASP | 110 | 44.519 | 59.891 | 64.140 | 1.00 | 11.53 | DIC |
| ATOM | 830 | CG | ASP | 110 | 45.831 | 60.491 | 64.648 | 1.00 | 16.88 | DIC |
| ATOM | 831 | OD1 | ASP | 110 | 46.727 | 60.759 | 63.819 | 1.00 | 17.49 | DIC |
| ATOM | 832 | OD2 | ASP | 110 | 45.962 | 60.717 | 65.873 | 1.00 | 16.60 | DIC |
| ATOM | 833 | C | ASP | 110 | 45.160 | 57.560 | 63.436 | 1.00 | 10.59 | DIC |
| ATOM | 834 | O | ASP | 110 | 44.371 | 56.613 | 63.457 | 1.00 | 9.32 | DIC |
| ATOM | 835 | N | ARG | 111 | 46.426 | 57.455 | 63.817 | 1.00 | 10.81 | DIC |
| ATOM | 836 | CA | ARG | 111 | 46.983 | 56.199 | 64.279 | 1.00 | 9.88 | DIC |
| ATOM | 837 | CB | ARG | 111 | 48.427 | 56.420 | 64.706 | 1.00 | 13.67 | DIC |
| ATOM | 838 | CG | ARG | 111 | 49.043 | 55.233 | 65.361 | 1.00 | 12.82 | DIC |
| ATOM | 839 | CD | ARG | 111 | 49.542 | 55.638 | 66.719 | 1.00 | 24.99 | DIC |
| ATOM | 840 | NE | ARG | 111 | 50.595 | 56.646 | 66.660 | 1.00 | 28.05 | DIC |
| ATOM | 841 | CZ | ARG | 111 | 51.187 | 57.155 | 67.733 | 1.00 | 29.20 | DIC |
| ATOM | 842 | NH1 | ARG | 111 | 50.825 | 56.747 | 68.943 | 1.00 | 29.64 | DIC |
| ATOM | 843 | NH2 | ARG | 111 | 52.141 | 58.069 | 67.597 | 1.00 | 32.61 | DIC |
| ATOM | 844 | C | ARG | 111 | 46.201 | 55.533 | 65.410 | 1.00 | 9.74 | DIC |
| ATOM | 845 | O | ARG | 111 | 45.718 | 56.189 | 66.330 | 1.00 | 6.00 | DIC |
| ATOM | 846 | N | LEU | 112 | 46.097 | 54.211 | 65.330 | 1.00 | 7.64 | DIC |
| ATOM | 847 | CA | LEU | 112 | 45.381 | 53.419 | 66.322 | 1.00 | 7.15 | DIC |
| ATOM | 848 | CB | LEU | 112 | 44.572 | 52.328 | 65.613 | 1.00 | 7.72 | DIC |
| ATOM | 849 | CG | LEU | 112 | 43.132 | 52.585 | 65.149 | 1.00 | 11.87 | DIC |
| ATOM | 850 | CD1 | LEU | 112 | 42.820 | 54.067 | 65.067 | 1.00 | 11.31 | DIC |
| ATOM | 851 | CD2 | LEU | 112 | 42.934 | 51.891 | 63.815 | 1.00 | 12.19 | DIC |
| ATOM | 852 | C | LEU | 112 | 46.294 | 52.759 | 67.358 | 1.00 | 6.68 | DIC |
| ATOM | 853 | O | LEU | 112 | 45.812 | 52.069 | 68.256 | 1.00 | 5.47 | DIC |
| ATOM | 854 | N | THR | 113 | 47.601 | 52.968 | 67.262 | 1.00 | 6.84 | DIC |
| ATOM | 855 | CA | THR | 113 | 48.491 | 52.305 | 68.211 | 1.00 | 9.74 | DIC |
| ATOM | 856 | CB | THR | 113 | 49.982 | 52.542 | 67.867 | 1.00 | 13.43 | DIC |
| ATOM | 857 | OG1 | THR | 113 | 50.395 | 53.834 | 68.316 | 1.00 | 20.21 | DIC |
| ATOM | 858 | CG2 | THR | 113 | 50.191 | 52.430 | 66.366 | 1.00 | 9.16 | DIC |
| ATOM | 859 | C | THR | 113 | 48.233 | 52.627 | 69.686 | 1.00 | 9.89 | DIC |
| ATOM | 860 | O | THR | 113 | 48.456 | 51.780 | 70.549 | 1.00 | 10.39 | DIC |
| ATOM | 861 | N | PRO | 114 | 47.763 | 53.847 | 70.005 | 1.00 | 10.53 | DIC |
| ATOM | 862 | CD | PRO | 114 | 47.662 | 55.093 | 69.228 | 1.00 | 10.25 | DIC |
| ATOM | 863 | CA | PRO | 114 | 47.521 | 54.104 | 71.428 | 1.00 | 9.66 | DIC |
| ATOM | 864 | CB | PRO | 114 | 47.058 | 55.558 | 71.440 | 1.00 | 10.92 | DIC |
| ATOM | 865 | CG | PRO | 114 | 47.832 | 56.151 | 70.300 | 1.00 | 12.85 | DIC |
| ATOM | 866 | C | PRO | 114 | 46.445 | 53.146 | 71.932 | 1.00 | 8.05 | DIC |
| ATOM | 867 | O | PRO | 114 | 46.529 | 52.620 | 73.039 | 1.00 | 6.80 | DIC |
| ATOM | 868 | N | LEU | 115 | 45.432 | 52.918 | 71.103 | 1.00 | 7.04 | DIC |
| ATOM | 869 | CA | LEU | 115 | 44.353 | 52.008 | 71.467 | 1.00 | 8.26 | DIC |
| ATOM | 870 | CB | LEU | 115 | 43.193 | 52.130 | 70.472 | 1.00 | 6.84 | DIC |
| ATOM | 871 | CG | LEU | 115 | 42.014 | 51.170 | 70.661 | 1.00 | 8.47 | DIC |
| ATOM | 872 | CD1 | LEU | 115 | 41.399 | 51.358 | 72.036 | 1.00 | 8.73 | DIC |
| ATOM | 873 | CD2 | LEU | 115 | 40.980 | 51.430 | 69.568 | 1.00 | 11.14 | DIC |
| ATOM | 874 | C | LEU | 115 | 44.897 | 50.582 | 71.471 | 1.00 | 5.85 | DIC |
| ATOM | 875 | O | LEU | 115 | 44.663 | 49.821 | 72.407 | 1.00 | 10.05 | DIC |
| ATOM | 876 | N | TRP | 116 | 45.634 | 50.238 | 70.422 | 1.00 | 6.30 | DIC |
| ATOM | 877 | CA | TRP | 116 | 46.233 | 48.914 | 70.284 | 1.00 | 7.03 | DIC |
| ATOM | 878 | CB | TRP | 116 | 47.005 | 48.836 | 68.962 | 1.00 | 5.84 | DIC |
| ATOM | 879 | CG | TRP | 116 | 47.720 | 47.527 | 68.737 | 1.00 | 8.60 | DIC |
| ATOM | 880 | CD2 | TRP | 116 | 47.120 | 46.266 | 68.423 | 1.00 | 7.22 | DIC |
| ATOM | 881 | CE2 | TRP | 116 | 48.165 | 45.319 | 68.322 | 1.00 | 8.24 | DIC |
| ATOM | 882 | CE3 | TRP | 116 | 45.797 | 45.841 | 68.221 | 1.00 | 7.76 | DIC |
| ATOM | 883 | CD1 | TRP | 116 | 49.067 | 47.303 | 68.810 | 1.00 | 8.45 | DIC |
| ATOM | 884 | NE1 | TRP | 116 | 49.342 | 45.980 | 68.562 | 1.00 | 8.62 | DIC |
| ATOM | 885 | CZ2 | TRP | 116 | 47.933 | 43.973 | 68.024 | 1.00 | 7.56 | DIC |
| ATOM | 886 | CZ3 | TRP | 116 | 45.565 | 44.501 | 67.923 | 1.00 | 9.68 | DIC |
| ATOM | 887 | CH2 | TRP | 116 | 46.632 | 43.581 | 67.829 | 1.00 | 9.41 | DIC |
| ATOM | 888 | C | TRP | 116 | 47.165 | 48.584 | 71.455 | 1.00 | 8.65 | DIC |
| ATOM | 889 | O | TRP | 116 | 47.199 | 47.448 | 71.926 | 1.00 | 8.41 | DIC |
| ATOM | 890 | N | ASP | 117 | 47.907 | 49.582 | 71.932 | 1.00 | 10.05 | DIC |
| ATOM | 891 | CA | ASP | 117 | 48.841 | 49.373 | 73.038 | 1.00 | 10.53 | DIC |
| ATOM | 892 | CB | ASP | 117 | 49.912 | 50.474 | 73.063 | 1.00 | 9.66 | DIC |
| ATOM | 893 | CG | ASP | 117 | 50.846 | 50.423 | 71.863 | 1.00 | 12.25 | DIC |
| ATOM | 894 | OD1 | ASP | 117 | 51.014 | 49.337 | 71.266 | 1.00 | 8.12 | DIC |
| ATOM | 895 | OD2 | ASP | 117 | 51.434 | 51.476 | 71.529 | 1.00 | 10.94 | DIC |
| ATOM | 896 | C | ASP | 117 | 48.168 | 49.313 | 74.409 | 1.00 | 12.03 | DIC |
| ATOM | 897 | O | ASP | 117 | 48.827 | 49.044 | 75.415 | 1.00 | 11.16 | DIC |
| ATOM | 898 | N | THR | 118 | 46.862 | 49.564 | 74.457 | 1.00 | 12.84 | DIC |
| ATOM | 899 | CA | THR | 118 | 46.141 | 49.536 | 75.727 | 1.00 | 13.69 | DIC |
| ATOM | 900 | CB | THR | 118 | 44.652 | 49.929 | 75.542 | 1.00 | 13.94 | DIC |
| ATOM | 901 | OG1 | THR | 118 | 44.568 | 51.266 | 75.027 | 1.00 | 13.81 | DIC |
| ATOM | 902 | CG2 | THR | 118 | 43.913 | 49.864 | 76.871 | 1.00 | 13.09 | DIC |
| ATOM | 903 | C | THR | 118 | 46.212 | 48.148 | 76.359 | 1.00 | 15.13 | DIC |
| ATOM | 904 | O | THR | 118 | 46.004 | 47.137 | 75.683 | 1.00 | 13.70 | DIC |
| ATOM | 905 | N | ASP | 119 | 46.523 | 48.102 | 77.652 | 1.00 | 16.15 | DIC |
| ATOM | 906 | CA | ASP | 119 | 46.608 | 46.831 | 78.368 | 1.00 | 18.15 | DIC |
| ATOM | 907 | CB | ASP | 119 | 47.565 | 46.945 | 79.557 | 1.00 | 22.51 | DIC |
| ATOM | 908 | CG | ASP | 119 | 47.891 | 45.597 | 80.177 | 1.00 | 26.80 | DIC |
| ATOM | 909 | OD1 | ASP | 119 | 46.976 | 44.757 | 80.303 | 1.00 | 29.43 | DIC |
| ATOM | 910 | OD2 | ASP | 119 | 49.064 | 45.378 | 80.549 | 1.00 | 32.86 | DIC |
| ATOM | 911 | C | ASP | 119 | 45.208 | 46.483 | 78.869 | 1.00 | 17.06 | DIC |
| ATOM | 912 | O | ASP | 119 | 44.670 | 47.164 | 79.740 | 1.00 | 16.61 | DIC |
| ATOM | 913 | N | LEU | 120 | 44.628 | 45.424 | 78.314 | 1.00 | 15.39 | DIC |
| ATOM | 914 | CA | LEU | 120 | 43.281 | 44.997 | 78.681 | 1.00 | 15.18 | DIC |
| ATOM | 915 | CB | LEU | 120 | 42.688 | 44.134 | 77.562 | 1.00 | 14.33 | DIC |
| ATOM | 916 | CG | LEU | 120 | 42.345 | 44.801 | 76.223 | 1.00 | 13.88 | DIC |
| ATOM | 917 | CD1 | LEU | 120 | 43.564 | 45.510 | 75.668 | 1.00 | 17.27 | DIC |
| ATOM | 918 | CD2 | LEU | 120 | 41.852 | 43.749 | 75.243 | 1.00 | 12.16 | DIC |
| ATOM | 919 | C | LEU | 120 | 43.205 | 44.227 | 79.998 | 1.00 | 15.94 | DIC |
| ATOM | 920 | O | LEU | 120 | 42.117 | 44.031 | 80.541 | 1.00 | 16.09 | DIC |
| ATOM | 921 | N | GLY | 121 | 44.349 | 43.789 | 80.513 | 1.00 | 15.28 | DIC |
| ATOM | 922 | CA | GLY | 121 | 44.329 | 43.033 | 81.752 | 1.00 | 16.86 | DIC |
| ATOM | 923 | C | GLY | 121 | 43.407 | 41.838 | 81.581 | 1.00 | 17.04 | DIC |
| ATOM | 924 | O | GLY | 121 | 43.440 | 41.174 | 80.542 | 1.00 | 16.37 | DIC |
| ATOM | 925 | N | ASN | 122 | 42.575 | 41.563 | 82.578 | 1.00 | 16.91 | DIC |
| ATOM | 926 | CA | ASN | 122 | 41.659 | 40.433 | 82.486 | 1.00 | 17.19 | DIC |
| ATOM | 927 | CB | ASN | 122 | 41.549 | 39.714 | 83.835 | 1.00 | 20.20 | DIC |
| ATOM | 928 | CG | ASN | 122 | 40.814 | 38.386 | 83.729 | 1.00 | 23.09 | DIC |
| ATOM | 929 | OD1 | ASN | 122 | 41.147 | 37.543 | 82.892 | 1.00 | 25.09 | DIC |
| ATOM | 930 | ND2 | ASN | 122 | 39.813 | 38.193 | 84.582 | 1.00 | 25.01 | DIC |
| ATOM | 931 | C | ASN | 122 | 40.275 | 40.868 | 82.014 | 1.00 | 15.72 | DIC |
| ATOM | 932 | O | ASN | 122 | 39.290 | 40.151 | 82.204 | 1.00 | 14.86 | DIC |
| ATOM | 933 | N | ASN | 123 | 40.201 | 42.053 | 81.415 | 1.00 | 13.48 | DIC |
| ATOM | 934 | CA | ASN | 123 | 38.937 | 42.556 | 80.891 | 1.00 | 12.64 | DIC |
| ATOM | 935 | CB | ASN | 123 | 39.026 | 44.057 | 80.579 | 1.00 | 12.19 | DIC |
| ATOM | 936 | CG | ASN | 123 | 39.078 | 44.921 | 81.831 | 1.00 | 15.92 | DIC |
| ATOM | 937 | OD1 | ASN | 123 | 40.093 | 45.563 | 82.122 | 1.00 | 15.70 | DIC |
| ATOM | 938 | ND2 | ASN | 123 | 37.983 | 44.940 | 82.576 | 1.00 | 13.08 | DIC |
| ATOM | 939 | C | ASN | 123 | 38.657 | 41.791 | 79.600 | 1.00 | 11.07 | DIC |
| ATOM | 940 | O | ASN | 123 | 39.582 | 41.276 | 78.971 | 1.00 | 10.73 | DIC |
| ATOM | 941 | N | TRP | 124 | 37.386 | 41.721 | 79.212 | 1.00 | 11.13 | DIC |
| ATOM | 942 | CA | TRP | 124 | 36.988 | 41.025 | 77.991 | 1.00 | 8.79 | DIC |
| ATOM | 943 | CB | TRP | 124 | 35.477 | 40.814 | 77.965 | 1.00 | 9.02 | DIC |
| ATOM | 944 | CG | TRP | 124 | 34.945 | 39.904 | 79.021 | 1.00 | 9.43 | DIC |
| ATOM | 945 | CD2 | TRP | 124 | 34.870 | 38.478 | 78.954 | 1.00 | 9.01 | DIC |
| ATOM | 946 | CE2 | TRP | 124 | 34.238 | 38.035 | 80.140 | 1.00 | 9.81 | DIC |
| ATOM | 947 | CE3 | TRP | 124 | 35.274 | 37.527 | 78.005 | 1.00 | 8.70 | DIC |
| ATOM | 948 | CD1 | TRP | 124 | 34.378 | 40.264 | 80.213 | 1.00 | 9.23 | DIC |
| ATOM | 949 | NE1 | TRP | 124 | 33.947 | 39.145 | 80.890 | 1.00 | 9.23 | DIC |
| ATOM | 950 | CZ2 | TRP | 124 | 33.997 | 36.681 | 80.401 | 1.00 | 9.69 | DIC |
| ATOM | 951 | CZ3 | TRP | 124 | 35.033 | 36.179 | 78.266 | 1.00 | 9.36 | DIC |
| ATOM | 952 | CH2 | TRP | 124 | 34.400 | 35.772 | 79.456 | 1.00 | 9.63 | DIC |
| ATOM | 953 | C | TRP | 124 | 37.381 | 41.804 | 76.735 | 1.00 | 8.82 | DIC |
| ATOM | 954 | O | TRP | 124 | 37.742 | 41.218 | 75.712 | 1.00 | 7.06 | DIC |
| ATOM | 955 | N | LEU | 125 | 37.294 | 43.125 | 76.812 | 1.00 | 7.98 | DIC |
| ATOM | 956 | CA | LEU | 125 | 37.630 | 43.966 | 75.669 | 1.00 | 9.33 | DIC |
| ATOM | 957 | CB | LEU | 125 | 36.580 | 43.793 | 74.563 | 1.00 | 7.77 | DIC |
| ATOM | 958 | CG | LEU | 125 | 35.173 | 44.370 | 74.791 | 1.00 | 11.95 | DIC |
| ATOM | 959 | CD1 | LEU | 125 | 34.233 | 43.857 | 73.703 | 1.00 | 11.77 | DIC |
| ATOM | 960 | CD2 | LEU | 125 | 34.641 | 43.968 | 76.154 | 1.00 | 15.27 | DIC |
| ATOM | 961 | C | LEU | 125 | 37.701 | 45.437 | 76.055 | 1.00 | 9.18 | DIC |
| ATOM | 962 | O | LEU | 125 | 37.325 | 45.826 | 77.160 | 1.00 | 9.87 | DIC |
| ATOM | 963 | N | GLY | 126 | 38.202 | 46.240 | 75.127 | 1.00 | 8.36 | DIC |
| ATOM | 964 | CA | GLY | 126 | 38.291 | 47.667 | 75.333 | 1.00 | 9.63 | DIC |
| ATOM | 965 | C | GLY | 126 | 37.379 | 48.269 | 74.284 | 1.00 | 9.92 | DIC |
| ATOM | 966 | O | GLY | 126 | 37.397 | 47.838 | 73.128 | 1.00 | 8.79 | DIC |
| ATOM | 967 | N | ALA | 127 | 36.566 | 49.243 | 74.674 | 1.00 | 8.77 | DIC |
| ATOM | 968 | CA | ALA | 127 | 35.650 | 49.870 | 73.726 | 1.00 | 7.45 | DIC |
| ATOM | 969 | CB | ALA | 127 | 34.386 | 49.022 | 73.594 | 1.00 | 7.33 | DIC |
| ATOM | 970 | C | ALA | 127 | 35.284 | 51.290 | 74.145 | 1.00 | 8.08 | DIC |
| ATOM | 971 | O | ALA | 127 | 35.462 | 51.675 | 75.305 | 1.00 | 5.69 | DIC |
| ATOM | 972 | N | SER | 128 | 34.787 | 52.069 | 73.189 | 1.00 | 6.63 | DIC |
| ATOM | 973 | CA | SER | 128 | 34.383 | 53.441 | 73.461 | 1.00 | 7.92 | DIC |
| ATOM | 974 | CB | SER | 128 | 34.631 | 54.327 | 72.233 | 1.00 | 8.31 | DIC |
| ATOM | 975 | OG | SER | 128 | 36.021 | 54.413 | 71.932 | 1.00 | 7.53 | DIC |
| ATOM | 976 | C | SER | 128 | 32.905 | 53.452 | 73.843 | 1.00 | 7.98 | DIC |
| ATOM | 977 | O | SER | 128 | 32.129 | 52.605 | 73.390 | 1.00 | 6.12 | DIC |
| ATOM | 978 | N | ILE | 129 | 32.529 | 54.411 | 74.683 | 1.00 | 8.45 | DIC |
| ATOM | 979 | CA | ILE | 129 | 31.157 | 54.547 | 75.165 | 1.00 | 7.19 | DIC |
| ATOM | 980 | CB | ILE | 129 | 31.110 | 55.479 | 76.406 | 1.00 | 8.54 | DIC |
| ATOM | 981 | CG2 | ILE | 129 | 29.667 | 55.842 | 76.752 | 1.00 | 9.06 | DIC |
| ATOM | 982 | CG1 | ILE | 129 | 31.815 | 54.799 | 77.586 | 1.00 | 8.78 | DIC |
| ATOM | 983 | CD | ILE | 129 | 31.972 | 55.685 | 78.804 | 1.00 | 8.26 | DIC |
| ATOM | 984 | C | ILE | 129 | 30.205 | 55.092 | 74.104 | 1.00 | 8.79 | DIC |
| ATOM | 985 | O | ILE | 129 | 30.572 | 55.954 | 73.305 | 1.00 | 6.50 | DIC |
| ATOM | 986 | N | ASP | 130 | 28.980 | 54.577 | 74.100 | 1.00 | 7.31 | DIC |
| ATOM | 987 | CA | ASP | 130 | 27.978 | 55.037 | 73.152 | 1.00 | 8.69 | DIC |
| ATOM | 988 | CB | ASP | 130 | 27.102 | 53.873 | 72.692 | 1.00 | 6.90 | DIC |
| ATOM | 989 | CG | ASP | 130 | 26.237 | 54.238 | 71.510 | 1.00 | 7.79 | DIC |
| ATOM | 990 | OD1 | ASP | 130 | 25.535 | 55.274 | 71.578 | 1.00 | 7.38 | DIC |
| ATOM | 991 | OD2 | ASP | 130 | 26.258 | 53.493 | 70.511 | 1.00 | 8.54 | DIC |
| ATOM | 992 | C | ASP | 130 | 27.125 | 56.078 | 73.871 | 1.00 | 8.24 | DIC |
| ATOM | 993 | O | ASP | 130 | 26.249 | 55.729 | 74.660 | 1.00 | 9.03 | DIC |
| ATOM | 994 | N | LEU | 131 | 27.398 | 57.353 | 73.608 | 1.00 | 10.56 | DIC |
| ATOM | 995 | CA | LEU | 131 | 26.668 | 58.456 | 74.242 | 1.00 | 11.56 | DIC |
| ATOM | 996 | CB | LEU | 131 | 27.234 | 59.808 | 73.785 | 1.00 | 11.69 | DIC |
| ATOM | 997 | CG | LEU | 131 | 28.607 | 60.232 | 74.318 | 1.00 | 15.03 | DIC |
| ATOM | 998 | CD1 | LEU | 131 | 29.662 | 59.190 | 73.969 | 1.00 | 17.38 | DIC |
| ATOM | 999 | CD2 | LEU | 131 | 28.981 | 61.583 | 73.722 | 1.00 | 15.49 | DIC |
| ATOM | 1000 | C | LEU | 131 | 25.168 | 58.428 | 73.974 | 1.00 | 11.77 | DIC |
| ATOM | 1001 | O | LEU | 131 | 24.367 | 58.755 | 74.853 | 1.00 | 10.73 | DIC |
| ATOM | 1002 | N | PHE | 132 | 24.789 | 58.045 | 72.760 | 1.00 | 11.04 | DIC |
| ATOM | 1003 | CA | PHE | 132 | 23.381 | 57.988 | 72.388 | 1.00 | 12.06 | DIC |
| ATOM | 1004 | CB | PHE | 132 | 23.244 | 57.642 | 70.903 | 1.00 | 14.10 | DIC |
| ATOM | 1005 | CG | PHE | 132 | 21.820 | 57.544 | 70.433 | 1.00 | 16.45 | DIC |
| ATOM | 1006 | CD1 | PHE | 132 | 21.121 | 56.346 | 70.531 | 1.00 | 16.62 | DIC |
| ATOM | 1007 | CD2 | PHE | 132 | 21.167 | 58.658 | 69.922 | 1.00 | 19.15 | DIC |
| ATOM | 1008 | CE1 | PHE | 132 | 19.793 | 56.257 | 70.128 | 1.00 | 16.52 | DIC |
| ATOM | 1009 | CE2 | PHE | 132 | 19.834 | 58.582 | 69.515 | 1.00 | 19.44 | DIC |
| ATOM | 1010 | CZ | PHE | 132 | 19.148 | 57.378 | 69.619 | 1.00 | 18.41 | DIC |
| ATOM | 1011 | C | PHE | 132 | 22.589 | 56.988 | 73.230 | 1.00 | 12.25 | DIC |
| ATOM | 1012 | O | PHE | 132 | 21.542 | 57.327 | 73.789 | 1.00 | 12.83 | DIC |
| ATOM | 1013 | N | VAL | 133 | 23.086 | 55.757 | 73.321 | 1.00 | 10.53 | DIC |
| ATOM | 1014 | CA | VAL | 133 | 22.404 | 54.722 | 74.093 | 1.00 | 10.89 | DIC |
| ATOM | 1015 | CB | VAL | 133 | 23.017 | 53.330 | 73.843 | 1.00 | 10.97 | DIC |
| ATOM | 1016 | CG1 | VAL | 133 | 22.268 | 52.280 | 74.669 | 1.00 | 12.18 | DIC |
| ATOM | 1017 | CG2 | VAL | 133 | 22.953 | 52.987 | 72.365 | 1.00 | 10.49 | DIC |
| ATOM | 1018 | C | VAL | 133 | 22.481 | 55.006 | 75.586 | 1.00 | 12.00 | DIC |
| ATOM | 1019 | O | VAL | 133 | 21.532 | 54.746 | 76.327 | 1.00 | 10.73 | DIC |
| ATOM | 1020 | N | GLU | 134 | 23.620 | 55.533 | 76.022 | 1.00 | 12.24 | DIC |
| ATOM | 1021 | CA | GLU | 134 | 23.822 | 55.843 | 77.431 | 1.00 | 14.14 | DIC |
| ATOM | 1022 | CB | GLU | 134 | 25.250 | 56.354 | 77.656 | 1.00 | 14.36 | DIC |
| ATOM | 1023 | CG | GLU | 134 | 25.592 | 56.614 | 79.114 | 1.00 | 16.20 | DIC |
| ATOM | 1024 | CD | GLU | 134 | 25.664 | 55.348 | 79.951 | 1.00 | 14.45 | DIC |
| ATOM | 1025 | OE1 | GLU | 134 | 25.645 | 55.468 | 81.189 | 1.00 | 17.32 | DIC |
| ATOM | 1026 | OE2 | GLU | 134 | 25.750 | 54.235 | 79.383 | 1.00 | 14.66 | DIC |
| ATOM | 1027 | C | GLU | 134 | 22.813 | 56.872 | 77.927 | 1.00 | 14.91 | DIC |
| ATOM | 1028 | O | GLU | 134 | 22.415 | 56.845 | 79.090 | 1.00 | 16.45 | DIC |
| ATOM | 1029 | N | ARG | 135 | 22.388 | 57.776 | 77.051 | 1.00 | 16.10 | DIC |
| ATOM | 1030 | CA | ARG | 135 | 21.428 | 58.792 | 77.454 | 1.00 | 16.53 | DIC |
| ATOM | 1031 | CB | ARG | 135 | 21.785 | 60.150 | 76.834 | 1.00 | 19.31 | DIC |
| ATOM | 1032 | CG | ARG | 135 | 21.548 | 60.279 | 75.341 | 1.00 | 22.07 | DIC |
| ATOM | 1033 | CD | ARG | 135 | 22.304 | 61.487 | 74.800 | 1.00 | 26.30 | DIC |
| ATOM | 1034 | NE | ARG | 135 | 21.972 | 62.716 | 75.516 | 1.00 | 29.13 | DIC |
| ATOM | 1035 | CZ | ARG | 135 | 20.851 | 63.410 | 75.339 | 1.00 | 32.01 | DIC |
| ATOM | 1036 | NH1 | ARG | 135 | 20.635 | 64.516 | 76.041 | 1.00 | 31.51 | DIC |
| ATOM | 1037 | NH2 | ARG | 135 | 19.950 | 63.007 | 74.450 | 1.00 | 32.01 | DIC |
| ATOM | 1038 | C | ARG | 135 | 19.992 | 58.405 | 77.110 | 1.00 | 17.25 | DIC |
| ATOM | 1039 | O | ARG | 135 | 19.078 | 59.217 | 77.232 | 1.00 | 17.38 | DIC |
| ATOM | 1040 | N | GLN | 136 | 19.800 | 57.164 | 76.674 | 1.00 | 15.73 | DIC |
| ATOM | 1041 | CA | GLN | 136 | 18.462 | 56.673 | 76.361 | 1.00 | 17.42 | DIC |
| ATOM | 1042 | CB | GLN | 136 | 18.538 | 55.479 | 75.413 | 1.00 | 16.78 | DIC |
| ATOM | 1043 | CG | GLN | 136 | 17.213 | 55.086 | 74.796 | 1.00 | 18.64 | DIC |
| ATOM | 1044 | CD | GLN | 136 | 17.355 | 53.916 | 73.845 | 1.00 | 21.61 | DIC |
| ATOM | 1045 | OE1 | GLN | 136 | 18.327 | 53.833 | 73.097 | 1.00 | 23.71 | DIC |
| ATOM | 1046 | NE2 | GLN | 136 | 16.383 | 53.012 | 73.861 | 1.00 | 21.40 | DIC |
| ATOM | 1047 | C | GLN | 136 | 17.905 | 56.236 | 77.713 | 1.00 | 18.33 | DIC |
| ATOM | 1048 | O | GLN | 136 | 18.123 | 55.106 | 78.149 | 1.00 | 16.98 | DIC |
| ATOM | 1049 | N | GLU | 137 | 17.199 | 57.146 | 78.375 | 1.00 | 20.02 | DIC |
| ATOM | 1050 | CA | GLU | 137 | 16.649 | 56.895 | 79.705 | 1.00 | 22.25 | DIC |
| ATOM | 1051 | CB | GLU | 137 | 15.653 | 58.004 | 80.072 | 1.00 | 26.10 | DIC |
| ATOM | 1052 | CG | GLU | 137 | 15.839 | 58.561 | 81.485 | 1.00 | 31.74 | DIC |
| ATOM | 1053 | CD | GLU | 137 | 15.083 | 57.774 | 82.544 | 1.00 | 35.77 | DIC |
| ATOM | 1054 | OE1 | GLU | 137 | 13.842 | 57.916 | 82.611 | 1.00 | 38.12 | DIC |
| ATOM | 1055 | OE2 | GLU | 137 | 15.723 | 57.016 | 83.308 | 1.00 | 36.14 | DIC |
| ATOM | 1056 | C | GLU | 137 | 16.008 | 55.529 | 79.927 | 1.00 | 20.26 | DIC |
| ATOM | 1057 | O | GLU | 137 | 15.064 | 55.145 | 79.234 | 1.00 | 21.74 | DIC |
| ATOM | 1058 | N | GLY | 138 | 16.547 | 54.798 | 80.898 | 1.00 | 19.39 | DIC |
| ATOM | 1059 | CA | GLY | 138 | 16.023 | 53.488 | 81.244 | 1.00 | 17.93 | DIC |
| ATOM | 1060 | C | GLY | 138 | 16.402 | 52.294 | 80.383 | 1.00 | 16.30 | DIC |
| ATOM | 1061 | O | GLY | 138 | 16.196 | 51.156 | 80.799 | 1.00 | 16.99 | DIC |
| ATOM | 1062 | N | TYR | 139 | 16.955 | 52.520 | 79.196 | 1.00 | 14.22 | DIC |
| ATOM | 1063 | CA | TYR | 139 | 17.303 | 51.392 | 78.340 | 1.00 | 12.95 | DIC |
| ATOM | 1064 | CB | TYR | 139 | 17.775 | 51.852 | 76.962 | 1.00 | 11.93 | DIC |
| ATOM | 1065 | CG | TYR | 139 | 18.077 | 50.670 | 76.066 | 1.00 | 9.65 | DIC |
| ATOM | 1066 | CD1 | TYR | 139 | 17.051 | 49.841 | 75.618 | 1.00 | 9.55 | DIC |
| ATOM | 1067 | CE1 | TYR | 139 | 17.318 | 48.699 | 74.870 | 1.00 | 6.57 | DIC |
| ATOM | 1068 | CD2 | TYR | 139 | 19.390 | 50.327 | 75.735 | 1.00 | 8.27 | DIC |
| ATOM | 1069 | CE2 | TYR | 139 | 19.668 | 49.180 | 74.983 | 1.00 | 6.71 | DIC |
| ATOM | 1070 | CZ | TYR | 139 | 18.622 | 48.372 | 74.558 | 1.00 | 6.55 | DIC |
| ATOM | 1071 | OH | TYR | 139 | 18.868 | 47.224 | 73.836 | 1.00 | 7.26 | DIC |
| ATOM | 1072 | C | TYR | 139 | 18.372 | 50.479 | 78.916 | 1.00 | 12.54 | DIC |
| ATOM | 1073 | O | TYR | 139 | 18.174 | 49.274 | 79.025 | 1.00 | 14.54 | DIC |
| ATOM | 1074 | N | LYS | 140 | 19.511 | 51.066 | 79.257 | 1.00 | 12.11 | DIC |
| ATOM | 1075 | CA | LYS | 140 | 20.643 | 50.335 | 79.801 | 1.00 | 12.14 | DIC |
| ATOM | 1076 | CB | LYS | 140 | 21.660 | 51.338 | 80.350 | 1.00 | 14.79 | DIC |
| ATOM | 1077 | CG | LYS | 140 | 22.981 | 50.743 | 80.761 | 1.00 | 17.75 | DIC |
| ATOM | 1078 | CD | LYS | 140 | 24.029 | 51.821 | 81.007 | 1.00 | 15.95 | DIC |
| ATOM | 1079 | CE | LYS | 140 | 23.711 | 52.669 | 82.224 | 1.00 | 17.64 | DIC |
| ATOM | 1080 | NZ | LYS | 140 | 24.887 | 53.502 | 82.602 | 1.00 | 16.20 | DIC |
| ATOM | 1081 | C | LYS | 140 | 20.229 | 49.344 | 80.884 | 1.00 | 13.45 | DIC |
| ATOM | 1082 | O | LYS | 140 | 20.690 | 48.201 | 80.903 | 1.00 | 13.33 | DIC |
| ATOM | 1083 | N | GLN | 141 | 19.348 | 49.776 | 81.778 | 1.00 | 11.99 | DIC |
| ATOM | 1084 | CA | GLN | 141 | 18.890 | 48.917 | 82.860 | 1.00 | 14.94 | DIC |
| ATOM | 1085 | CB | GLN | 141 | 18.150 | 49.752 | 83.914 | 1.00 | 15.97 | DIC |
| ATOM | 1086 | CG | GLN | 141 | 19.049 | 50.738 | 84.676 | 1.00 | 16.22 | DIC |
| ATOM | 1087 | CD | GLN | 141 | 19.452 | 51.962 | 83.859 | 1.00 | 19.15 | DIC |
| ATOM | 1088 | OE1 | GLN | 141 | 19.056 | 52.118 | 82.702 | 1.00 | 20.43 | DIC |
| ATOM | 1089 | NE2 | GLN | 141 | 20.240 | 52.843 | 84.469 | 1.00 | 16.02 | DIC |
| ATOM | 1090 | C | GLN | 141 | 18.008 | 47.766 | 82.369 | 1.00 | 14.90 | DIC |
| ATOM | 1091 | O | GLN | 141 | 17.990 | 46.686 | 82.968 | 1.00 | 15.34 | DIC |
| ATOM | 1092 | N | LYS | 142 | 17.284 | 47.992 | 81.278 | 1.00 | 14.30 | DIC |
| ATOM | 1093 | CA | LYS | 142 | 16.427 | 46.957 | 80.716 | 1.00 | 15.53 | DIC |
| ATOM | 1094 | CB | LYS | 142 | 15.698 | 47.475 | 79.470 | 1.00 | 17.58 | DIC |
| ATOM | 1095 | CG | LYS | 142 | 14.482 | 48.333 | 79.757 | 1.00 | 21.30 | DIC |
| ATOM | 1096 | CD | LYS | 142 | 13.895 | 48.879 | 78.459 | 1.00 | 25.10 | DIC |
| ATOM | 1097 | CE | LYS | 142 | 12.527 | 49.508 | 78.678 | 1.00 | 28.09 | DIC |
| ATOM | 1098 | NZ | LYS | 142 | 11.484 | 48.490 | 78.984 | 1.00 | 31.88 | DIC |
| ATOM | 1099 | C | LYS | 142 | 17.229 | 45.709 | 80.351 | 1.00 | 14.42 | DIC |
| ATOM | 1100 | O | LYS | 142 | 16.684 | 44.606 | 80.317 | 1.00 | 15.12 | DIC |
| ATOM | 1101 | N | ILE | 143 | 18.517 | 45.870 | 80.060 | 1.00 | 12.66 | DIC |
| ATOM | 1102 | CA | ILE | 143 | 19.319 | 44.699 | 79.724 | 1.00 | 11.78 | DIC |
| ATOM | 1103 | CB | ILE | 143 | 20.131 | 44.898 | 78.421 | 1.00 | 11.52 | DIC |
| ATOM | 1104 | CG2 | ILE | 143 | 19.177 | 45.095 | 77.249 | 1.00 | 9.94 | DIC |
| ATOM | 1105 | CG1 | ILE | 143 | 21.084 | 46.085 | 78.550 | 1.00 | 10.92 | DIC |
| ATOM | 1106 | CD | ILE | 143 | 22.037 | 46.202 | 77.376 | 1.00 | 9.97 | DIC |
| ATOM | 1107 | C | ILE | 143 | 20.246 | 44.268 | 80.863 | 1.00 | 11.61 | DIC |
| ATOM | 1108 | O | ILE | 143 | 21.264 | 43.612 | 80.639 | 1.00 | 11.97 | DIC |
| ATOM | 1109 | N | GLY | 144 | 19.873 | 44.649 | 82.085 | 1.00 | 11.10 | DIC |
| ATOM | 1110 | CA | GLY | 144 | 20.624 | 44.268 | 83.270 | 1.00 | 9.94 | DIC |
| ATOM | 1111 | C | GLY | 144 | 21.853 | 45.060 | 83.663 | 1.00 | 11.04 | DIC |
| ATOM | 1112 | O | GLY | 144 | 22.626 | 44.621 | 84.518 | 1.00 | 9.79 | DIC |
| ATOM | 1113 | N | MSE | 145 | 22.048 | 46.227 | 83.062 | 1.00 | 10.12 | DIC |
| ATOM | 1114 | CA | MSE | 145 | 23.212 | 47.031 | 83.390 | 1.00 | 11.90 | DIC |
| ATOM | 1115 | CB | MSE | 145 | 23.725 | 47.738 | 82.135 | 1.00 | 12.20 | DIC |
| ATOM | 1116 | CG | MSE | 145 | 24.329 | 46.789 | 81.111 | 1.00 | 13.32 | DIC |
| ATOM | 1117 | SE | MSE | 145 | 24.908 | 47.720 | 79.517 | 1.00 | 19.71 | DIC |
| ATOM | 1118 | CE | MSE | 145 | 26.468 | 48.596 | 80.243 | 1.00 | 14.79 | DIC |
| ATOM | 1119 | C | MSE | 145 | 22.931 | 48.048 | 84.487 | 1.00 | 12.83 | DIC |
| ATOM | 1120 | O | MSE | 145 | 21.821 | 48.576 | 84.594 | 1.00 | 11.70 | DIC |
| ATOM | 1121 | N | ALA | 146 | 23.948 | 48.314 | 85.301 | 1.00 | 14.28 | DIC |
| ATOM | 1122 | CA | ALA | 146 | 23.832 | 49.274 | 86.395 | 1.00 | 14.92 | DIC |
| ATOM | 1123 | CB | ALA | 146 | 24.793 | 48.903 | 87.517 | 1.00 | 14.61 | DIC |
| ATOM | 1124 | C | ALA | 146 | 24.140 | 50.677 | 85.891 | 1.00 | 15.53 | DIC |
| ATOM | 1125 | O | ALA | 146 | 24.776 | 50.843 | 84.852 | 1.00 | 14.24 | DIC |
| ATOM | 1126 | N | ASP | 147 | 23.688 | 51.683 | 86.634 | 1.00 | 16.07 | DIC |
| ATOM | 1127 | CA | ASP | 147 | 23.917 | 53.075 | 86.262 | 1.00 | 16.26 | DIC |
| ATOM | 1128 | CB | ASP | 147 | 23.370 | 54.011 | 87.346 | 1.00 | 20.59 | DIC |
| ATOM | 1129 | CG | ASP | 147 | 23.700 | 55.473 | 87.078 | 1.00 | 23.36 | DIC |
| ATOM | 1130 | OD1 | ASP | 147 | 23.214 | 56.026 | 86.069 | 1.00 | 25.96 | DIC |
| ATOM | 1131 | OD2 | ASP | 147 | 24.451 | 56.071 | 87.878 | 1.00 | 28.48 | DIC |
| ATOM | 1132 | C | ASP | 147 | 25.400 | 53.352 | 86.066 | 1.00 | 14.79 | DIC |
| ATOM | 1133 | O | ASP | 147 | 25.780 | 54.135 | 85.194 | 1.00 | 13.86 | DIC |
| ATOM | 1134 | N | GLY | 148 | 26.230 | 52.697 | 86.876 | 1.00 | 13.02 | DIC |
| ATOM | 1135 | CA | GLY | 148 | 27.668 | 52.894 | 86.806 | 1.00 | 11.35 | DIC |
| ATOM | 1136 | C | GLY | 148 | 28.396 | 52.094 | 85.744 | 1.00 | 10.20 | DIC |
| ATOM | 1137 | O | GLY | 148 | 29.606 | 52.246 | 85.574 | 1.00 | 11.09 | DIC |
| ATOM | 1138 | N | GLU | 149 | 27.676 | 51.225 | 85.043 | 1.00 | 9.04 | DIC |
| ATOM | 1139 | CA | GLU | 149 | 28.277 | 50.429 | 83.979 | 1.00 | 9.03 | DIC |
| ATOM | 1140 | CB | GLU | 149 | 27.733 | 49.001 | 84.003 | 1.00 | 8.78 | DIC |
| ATOM | 1141 | CG | GLU | 149 | 27.982 | 48.306 | 85.330 | 1.00 | 12.20 | DIC |
| ATOM | 1142 | CD | GLU | 149 | 27.390 | 46.916 | 85.382 | 1.00 | 11.83 | DIC |
| ATOM | 1143 | OE1 | GLU | 149 | 26.235 | 46.747 | 84.947 | 1.00 | 12.11 | DIC |
| ATOM | 1144 | OE2 | GLU | 149 | 28.073 | 45.994 | 85.871 | 1.00 | 16.06 | DIC |
| ATOM | 1145 | C | GLU | 149 | 27.884 | 51.142 | 82.697 | 1.00 | 8.47 | DIC |
| ATOM | 1146 | O | GLU | 149 | 26.701 | 51.294 | 82.393 | 1.00 | 9.02 | DIC |
| ATOM | 1147 | N | TYR | 150 | 28.886 | 51.591 | 81.956 | 1.00 | 8.23 | DIC |
| ATOM | 1148 | CA | TYR | 150 | 28.645 | 52.343 | 80.738 | 1.00 | 9.12 | DIC |
| ATOM | 1149 | CB | TYR | 150 | 29.760 | 53.383 | 80.590 | 1.00 | 8.51 | DIC |
| ATOM | 1150 | CG | TYR | 150 | 29.912 | 54.216 | 81.854 | 1.00 | 9.43 | DIC |
| ATOM | 1151 | CD1 | TYR | 150 | 31.155 | 54.388 | 82.461 | 1.00 | 10.16 | DIC |
| ATOM | 1152 | CE1 | TYR | 150 | 31.283 | 55.106 | 83.654 | 1.00 | 10.95 | DIC |
| ATOM | 1153 | CD2 | TYR | 150 | 28.796 | 54.790 | 82.469 | 1.00 | 12.27 | DIC |
| ATOM | 1154 | CE2 | TYR | 150 | 28.913 | 55.511 | 83.662 | 1.00 | 10.90 | DIC |
| ATOM | 1155 | CZ | TYR | 150 | 30.160 | 55.661 | 84.248 | 1.00 | 13.06 | DIC |
| ATOM | 1156 | OH | TYR | 150 | 30.279 | 56.354 | 85.433 | 1.00 | 12.48 | DIC |
| ATOM | 1157 | C | TYR | 150 | 28.488 | 51.494 | 79.487 | 1.00 | 7.57 | DIC |
| ATOM | 1158 | O | TYR | 150 | 29.309 | 50.625 | 79.187 | 1.00 | 6.61 | DIC |
| ATOM | 1159 | N | TYR | 151 | 27.401 | 51.755 | 78.769 | 1.00 | 7.71 | DIC |
| ATOM | 1160 | CA | TYR | 151 | 27.069 | 51.035 | 77.547 | 1.00 | 7.79 | DIC |
| ATOM | 1161 | CB | TYR | 151 | 25.629 | 51.375 | 77.144 | 1.00 | 6.89 | DIC |
| ATOM | 1162 | CG | TYR | 151 | 25.057 | 50.553 | 76.009 | 1.00 | 7.54 | DIC |
| ATOM | 1163 | CD1 | TYR | 151 | 25.432 | 50.789 | 74.685 | 1.00 | 5.74 | DIC |
| ATOM | 1164 | CE1 | TYR | 151 | 24.889 | 50.039 | 73.635 | 1.00 | 4.54 | DIC |
| ATOM | 1165 | CD2 | TYR | 151 | 24.125 | 49.546 | 76.260 | 1.00 | 7.45 | DIC |
| ATOM | 1166 | CE2 | TYR | 151 | 23.579 | 48.792 | 75.222 | 1.00 | 6.69 | DIC |
| ATOM | 1167 | CZ | TYR | 151 | 23.964 | 49.042 | 73.913 | 1.00 | 6.52 | DIC |
| ATOM | 1168 | OH | TYR | 151 | 23.432 | 48.282 | 72.891 | 1.00 | 6.05 | DIC |
| ATOM | 1169 | C | TYR | 151 | 28.047 | 51.443 | 76.454 | 1.00 | 6.36 | DIC |
| ATOM | 1170 | O | TYR | 151 | 28.148 | 52.621 | 76.116 | 1.00 | 7.50 | DIC |
| ATOM | 1171 | N | PHE | 152 | 28.776 | 50.475 | 75.909 | 1.00 | 5.53 | DIC |
| ATOM | 1172 | CA | PHE | 152 | 29.745 | 50.779 | 74.870 | 1.00 | 4.94 | DIC |
| ATOM | 1173 | CB | PHE | 152 | 31.060 | 50.016 | 75.108 | 1.00 | 4.86 | DIC |
| ATOM | 1174 | CG | PHE | 152 | 30.919 | 48.513 | 75.089 | 1.00 | 6.40 | DIC |
| ATOM | 1175 | CD1 | PHE | 152 | 30.724 | 47.802 | 76.271 | 1.00 | 6.43 | DIC |
| ATOM | 1176 | CD2 | PHE | 152 | 30.984 | 47.808 | 73.888 | 1.00 | 4.17 | DIC |
| ATOM | 1177 | CE1 | PHE | 152 | 30.594 | 46.408 | 76.259 | 1.00 | 5.71 | DIC |
| ATOM | 1178 | CE2 | PHE | 152 | 30.854 | 46.416 | 73.865 | 1.00 | 5.71 | DIC |
| ATOM | 1179 | CZ | PHE | 152 | 30.659 | 45.717 | 75.055 | 1.00 | 5.50 | DIC |
| ATOM | 1180 | C | PHE | 152 | 29.229 | 50.481 | 73.470 | 1.00 | 5.82 | DIC |
| ATOM | 1181 | O | PHE | 152 | 28.301 | 49.695 | 73.290 | 1.00 | 5.93 | DIC |
| ATOM | 1182 | N | ASN | 153 | 29.828 | 51.133 | 72.481 | 1.00 | 5.20 | DIC |
| ATOM | 1183 | CA | ASN | 153 | 29.447 | 50.910 | 71.094 | 1.00 | 5.79 | DIC |
| ATOM | 1184 | CB | ASN | 153 | 29.685 | 52.176 | 70.260 | 1.00 | 6.58 | DIC |
| ATOM | 1185 | CG | ASN | 153 | 29.282 | 51.998 | 68.809 | 1.00 | 6.46 | DIC |
| ATOM | 1186 | OD1 | ASN | 153 | 30.095 | 51.621 | 67.974 | 1.00 | 6.69 | DIC |
| ATOM | 1187 | ND2 | ASN | 153 | 28.011 | 52.247 | 68.510 | 1.00 | 9.09 | DIC |
| ATOM | 1188 | C | ASN | 153 | 30.296 | 49.752 | 70.585 | 1.00 | 5.25 | DIC |
| ATOM | 1189 | O | ASN | 153 | 31.492 | 49.683 | 70.861 | 1.00 | 3.87 | DIC |
| ATOM | 1190 | N | ALA | 154 | 29.674 | 48.838 | 69.847 | 1.00 | 5.40 | DIC |
| ATOM | 1191 | CA | ALA | 154 | 30.373 | 47.661 | 69.336 | 1.00 | 7.13 | DIC |
| ATOM | 1192 | CB | ALA | 154 | 29.381 | 46.508 | 69.188 | 1.00 | 4.44 | DIC |
| ATOM | 1193 | C | ALA | 154 | 31.134 | 47.859 | 68.024 | 1.00 | 6.42 | DIC |
| ATOM | 1194 | O | ALA | 154 | 31.733 | 46.913 | 67.507 | 1.00 | 7.14 | DIC |
| ATOM | 1195 | N | GLY | 155 | 31.118 | 49.080 | 67.496 | 1.00 | 6.03 | DIC |
| ATOM | 1196 | CA | GLY | 155 | 31.792 | 49.359 | 66.239 | 1.00 | 4.30 | DIC |
| ATOM | 1197 | C | GLY | 155 | 33.312 | 49.368 | 66.250 | 1.00 | 6.06 | DIC |
| ATOM | 1198 | O | GLY | 155 | 33.939 | 49.273 | 65.192 | 1.00 | 4.92 | DIC |
| ATOM | 1199 | N | VAL | 156 | 33.907 | 49.496 | 67.433 | 1.00 | 4.75 | DIC |
| ATOM | 1200 | CA | VAL | 156 | 35.362 | 49.510 | 67.582 | 1.00 | 4.31 | DIC |
| ATOM | 1201 | CB | VAL | 156 | 35.901 | 50.954 | 67.701 | 1.00 | 5.59 | DIC |
| ATOM | 1202 | CG1 | VAL | 156 | 37.398 | 50.931 | 67.984 | 1.00 | 5.38 | DIC |
| ATOM | 1203 | CG2 | VAL | 156 | 35.617 | 51.727 | 66.405 | 1.00 | 2.76 | DIC |
| ATOM | 1204 | C | VAL | 156 | 35.686 | 48.741 | 68.855 | 1.00 | 5.47 | DIC |
| ATOM | 1205 | O | VAL | 156 | 35.396 | 49.203 | 69.959 | 1.00 | 5.04 | DIC |
| ATOM | 1206 | N | LEU | 157 | 36.300 | 47.573 | 68.703 | 1.00 | 4.91 | DIC |
| ATOM | 1207 | CA | LEU | 157 | 36.599 | 46.741 | 69.858 | 1.00 | 6.02 | DIC |
| ATOM | 1208 | CB | LEU | 157 | 35.606 | 45.580 | 69.908 | 1.00 | 5.28 | DIC |
| ATOM | 1209 | CG | LEU | 157 | 34.120 | 45.923 | 69.800 | 1.00 | 5.55 | DIC |
| ATOM | 1210 | CD1 | LEU | 157 | 33.324 | 44.655 | 69.516 | 1.00 | 3.12 | DIC |
| ATOM | 1211 | CD2 | LEU | 157 | 33.658 | 46.599 | 71.084 | 1.00 | 3.69 | DIC |
| ATOM | 1212 | C | LEU | 157 | 37.998 | 46.157 | 69.915 | 1.00 | 6.93 | DIC |
| ATOM | 1213 | O | LEU | 157 | 38.403 | 45.431 | 69.008 | 1.00 | 7.95 | DIC |
| ATOM | 1214 | N | LEU | 158 | 38.733 | 46.474 | 70.978 | 1.00 | 6.51 | DIC |
| ATOM | 1215 | CA | LEU | 158 | 40.059 | 45.897 | 71.174 | 1.00 | 6.78 | DIC |
| ATOM | 1216 | CB | LEU | 158 | 40.956 | 46.826 | 71.995 | 1.00 | 8.24 | DIC |
| ATOM | 1217 | CG | LEU | 158 | 42.416 | 46.378 | 72.110 | 1.00 | 8.34 | DIC |
| ATOM | 1218 | CD1 | LEU | 158 | 43.062 | 46.337 | 70.728 | 1.00 | 8.63 | DIC |
| ATOM | 1219 | CD2 | LEU | 158 | 43.164 | 47.331 | 73.018 | 1.00 | 9.77 | DIC |
| ATOM | 1220 | C | LEU | 158 | 39.693 | 44.652 | 71.978 | 1.00 | 7.75 | DIC |
| ATOM | 1221 | O | LEU | 158 | 39.304 | 44.740 | 73.146 | 1.00 | 5.84 | DIC |
| ATOM | 1222 | N | ILE | 159 | 39.803 | 43.493 | 71.343 | 1.00 | 7.13 | DIC |
| ATOM | 1223 | CA | ILE | 159 | 39.401 | 42.246 | 71.973 | 1.00 | 7.06 | DIC |
| ATOM | 1224 | CB | ILE | 159 | 38.582 | 41.418 | 70.951 | 1.00 | 8.06 | DIC |
| ATOM | 1225 | CG2 | ILE | 159 | 38.329 | 40.009 | 71.467 | 1.00 | 5.92 | DIC |
| ATOM | 1226 | CG1 | ILE | 159 | 37.274 | 42.160 | 70.652 | 1.00 | 7.50 | DIC |
| ATOM | 1227 | CD | ILE | 159 | 36.439 | 41.533 | 69.576 | 1.00 | 12.31 | DIC |
| ATOM | 1228 | C | ILE | 159 | 40.494 | 41.377 | 72.585 | 1.00 | 8.00 | DIC |
| ATOM | 1229 | O | ILE | 159 | 41.513 | 41.099 | 71.958 | 1.00 | 5.43 | DIC |
| ATOM | 1230 | N | ASN | 160 | 40.261 | 40.952 | 73.824 | 1.00 | 8.08 | DIC |
| ATOM | 1231 | CA | ASN | 160 | 41.198 | 40.079 | 74.530 | 1.00 | 8.60 | DIC |
| ATOM | 1232 | CB | ASN | 160 | 40.987 | 40.197 | 76.037 | 1.00 | 9.75 | DIC |
| ATOM | 1233 | CG | ASN | 160 | 41.948 | 39.334 | 76.838 | 1.00 | 8.59 | DIC |
| ATOM | 1234 | OD1 | ASN | 160 | 42.542 | 38.393 | 76.317 | 1.00 | 6.73 | DIC |
| ATOM | 1235 | ND2 | ASN | 160 | 42.092 | 39.648 | 78.119 | 1.00 | 8.57 | DIC |
| ATOM | 1236 | C | ASN | 160 | 40.857 | 38.665 | 74.066 | 1.00 | 9.81 | DIC |
| ATOM | 1237 | O | ASN | 160 | 40.217 | 37.898 | 74.793 | 1.00 | 9.36 | DIC |
| ATOM | 1238 | N | LEU | 161 | 41.276 | 38.331 | 72.849 | 1.00 | 11.11 | DIC |
| ATOM | 1239 | CA | LEU | 161 | 40.987 | 37.024 | 72.268 | 1.00 | 11.84 | DIC |
| ATOM | 1240 | CB | LEU | 161 | 41.603 | 36.905 | 70.871 | 1.00 | 13.69 | DIC |
| ATOM | 1241 | CG | LEU | 161 | 40.670 | 37.132 | 69.680 | 1.00 | 18.63 | DIC |
| ATOM | 1242 | CD1 | LEU | 161 | 41.441 | 36.900 | 68.392 | 1.00 | 17.69 | DIC |
| ATOM | 1243 | CD2 | LEU | 161 | 39.479 | 36.185 | 69.753 | 1.00 | 17.40 | DIC |
| ATOM | 1244 | C | LEU | 161 | 41.423 | 35.830 | 73.104 | 1.00 | 12.22 | DIC |
| ATOM | 1245 | O | LEU | 161 | 40.733 | 34.811 | 73.132 | 1.00 | 10.35 | DIC |
| ATOM | 1246 | N | LYS | 162 | 42.568 | 35.935 | 73.769 | 1.00 | 12.29 | DIC |
| ATOM | 1247 | CA | LYS | 162 | 43.031 | 34.823 | 74.591 | 1.00 | 13.27 | DIC |
| ATOM | 1248 | CB | LYS | 162 | 44.369 | 35.159 | 75.261 | 1.00 | 16.11 | DIC |
| ATOM | 1249 | CG | LYS | 162 | 44.829 | 34.102 | 76.258 | 1.00 | 20.91 | DIC |
| ATOM | 1250 | CD | LYS | 162 | 46.278 | 34.286 | 76.686 | 1.00 | 24.43 | DIC |
| ATOM | 1251 | CE | LYS | 162 | 47.247 | 33.739 | 75.643 | 1.00 | 27.26 | DIC |
| ATOM | 1252 | NZ | LYS | 162 | 48.647 | 33.711 | 76.161 | 1.00 | 27.23 | DIC |
| ATOM | 1253 | C | LYS | 162 | 41.975 | 34.491 | 75.643 | 1.00 | 12.71 | DIC |
| ATOM | 1254 | O | LYS | 162 | 41.677 | 33.325 | 75.878 | 1.00 | 13.95 | DIC |
| ATOM | 1255 | N | LYS | 163 | 41.400 | 35.516 | 76.268 | 1.00 | 12.30 | DIC |
| ATOM | 1256 | CA | LYS | 163 | 40.369 | 35.287 | 77.275 | 1.00 | 10.73 | DIC |
| ATOM | 1257 | CB | LYS | 163 | 40.006 | 36.594 | 77.987 | 1.00 | 9.94 | DIC |
| ATOM | 1258 | CG | LYS | 163 | 38.984 | 36.419 | 79.113 | 1.00 | 9.80 | DIC |
| ATOM | 1259 | CD | LYS | 163 | 38.667 | 37.744 | 79.792 | 1.00 | 12.02 | DIC |
| ATOM | 1260 | CE | LYS | 163 | 37.685 | 37.563 | 80.946 | 1.00 | 11.59 | DIC |
| ATOM | 1261 | NZ | LYS | 163 | 38.230 | 36.656 | 81.994 | 1.00 | 15.21 | DIC |
| ATOM | 1262 | C | LYS | 163 | 39.120 | 34.695 | 76.616 | 1.00 | 10.32 | DIC |
| ATOM | 1263 | O | LYS | 163 | 38.527 | 33.748 | 77.133 | 1.00 | 10.04 | DIC |
| ATOM | 1264 | N | TRP | 164 | 38.718 | 35.260 | 75.481 | 1.00 | 10.10 | DIC |
| ATOM | 1265 | CA | TRP | 164 | 37.545 | 34.768 | 74.762 | 1.00 | 10.66 | DIC |
| ATOM | 1266 | CB | TRP | 164 | 37.366 | 35.524 | 73.443 | 1.00 | 8.99 | DIC |
| ATOM | 1267 | CG | TRP | 164 | 36.729 | 36.883 | 73.567 | 1.00 | 7.04 | DIC |
| ATOM | 1268 | CD2 | TRP | 164 | 35.780 | 37.469 | 72.665 | 1.00 | 6.04 | DIC |
| ATOM | 1269 | CE2 | TRP | 164 | 35.516 | 38.777 | 73.127 | 1.00 | 5.54 | DIC |
| ATOM | 1270 | CE3 | TRP | 164 | 35.131 | 37.015 | 71.508 | 1.00 | 5.98 | DIC |
| ATOM | 1271 | CD1 | TRP | 164 | 36.989 | 37.829 | 74.514 | 1.00 | 7.19 | DIC |
| ATOM | 1272 | NE1 | TRP | 164 | 36.265 | 38.970 | 74.257 | 1.00 | 6.74 | DIC |
| ATOM | 1273 | CZ2 | TRP | 164 | 34.630 | 39.642 | 72.471 | 1.00 | 7.14 | DIC |
| ATOM | 1274 | CZ3 | TRP | 164 | 34.249 | 37.873 | 70.856 | 1.00 | 6.29 | DIC |
| ATOM | 1275 | CH2 | TRP | 164 | 34.008 | 39.174 | 71.342 | 1.00 | 6.21 | DIC |
| ATOM | 1276 | C | TRP | 164 | 37.657 | 33.271 | 74.464 | 1.00 | 11.95 | DIC |
| ATOM | 1277 | O | TRP | 164 | 36.695 | 32.519 | 74.642 | 1.00 | 9.24 | DIC |
| ATOM | 1278 | N | ARG | 165 | 38.829 | 32.841 | 74.005 | 1.00 | 11.53 | DIC |
| ATOM | 1279 | CA | ARG | 165 | 39.025 | 31.433 | 73.677 | 1.00 | 12.97 | DIC |
| ATOM | 1280 | CB | ARG | 165 | 40.392 | 31.220 | 73.011 | 1.00 | 13.33 | DIC |
| ATOM | 1281 | CG | ARG | 165 | 40.431 | 31.666 | 71.546 | 1.00 | 16.11 | DIC |
| ATOM | 1282 | CD | ARG | 165 | 41.607 | 31.041 | 70.792 | 1.00 | 16.25 | DIC |
| ATOM | 1283 | NE | ARG | 165 | 42.889 | 31.519 | 71.293 | 1.00 | 16.10 | DIC |
| ATOM | 1284 | CZ | ARG | 165 | 43.467 | 32.653 | 70.914 | 1.00 | 16.79 | DIC |
| ATOM | 1285 | NH1 | ARG | 165 | 44.632 | 33.005 | 71.440 | 1.00 | 19.33 | DIC |
| ATOM | 1286 | NH2 | ARG | 165 | 42.898 | 33.423 | 69.994 | 1.00 | 15.46 | DIC |
| ATOM | 1287 | C | ARG | 165 | 38.858 | 30.495 | 74.871 | 1.00 | 13.30 | DIC |
| ATOM | 1288 | O | ARG | 165 | 38.699 | 29.292 | 74.698 | 1.00 | 13.71 | DIC |
| ATOM | 1289 | N | ARG | 166 | 38.876 | 31.041 | 76.081 | 1.00 | 14.72 | DIC |
| ATOM | 1290 | CA | ARG | 166 | 38.699 | 30.218 | 77.277 | 1.00 | 14.91 | DIC |
| ATOM | 1291 | CB | ARG | 166 | 39.333 | 30.891 | 78.497 | 1.00 | 16.73 | DIC |
| ATOM | 1292 | CG | ARG | 166 | 40.852 | 30.924 | 78.497 | 1.00 | 18.26 | DIC |
| ATOM | 1293 | CD | ARG | 166 | 41.361 | 31.920 | 79.529 | 1.00 | 19.78 | DIC |
| ATOM | 1294 | NE | ARG | 166 | 42.817 | 32.015 | 79.525 | 1.00 | 23.68 | DIC |
| ATOM | 1295 | CZ | ARG | 166 | 43.494 | 33.142 | 79.725 | 1.00 | 22.99 | DIC |
| ATOM | 1296 | NH1 | ARG | 166 | 42.847 | 34.279 | 79.943 | 1.00 | 23.36 | DIC |
| ATOM | 1297 | NH2 | ARG | 166 | 44.820 | 33.133 | 79.697 | 1.00 | 25.53 | DIC |
| ATOM | 1298 | C | ARG | 166 | 37.213 | 29.999 | 77.558 | 1.00 | 14.47 | DIC |
| ATOM | 1299 | O | ARG | 166 | 36.854 | 29.254 | 78.463 | 1.00 | 14.64 | DIC |
| ATOM | 1300 | N | HIS | 167 | 36.352 | 30.640 | 76.776 | 1.00 | 13.19 | DIC |
| ATOM | 1301 | CA | HIS | 167 | 34.912 | 30.523 | 76.990 | 1.00 | 13.58 | DIC |
| ATOM | 1302 | CB | HIS | 167 | 34.380 | 31.815 | 77.624 | 1.00 | 14.56 | DIC |
| ATOM | 1303 | CG | HIS | 167 | 35.031 | 32.166 | 78.928 | 1.00 | 15.72 | DIC |
| ATOM | 1304 | CD2 | HIS | 167 | 36.121 | 32.920 | 79.206 | 1.00 | 14.81 | DIC |
| ATOM | 1305 | ND1 | HIS | 167 | 34.570 | 31.700 | 80.141 | 1.00 | 15.43 | DIC |
| ATOM | 1306 | CE1 | HIS | 167 | 35.347 | 32.151 | 81.109 | 1.00 | 14.64 | DIC |
| ATOM | 1307 | NE2 | HIS | 167 | 36.296 | 32.895 | 80.568 | 1.00 | 15.87 | DIC |
| ATOM | 1308 | C | HIS | 167 | 34.145 | 30.251 | 75.701 | 1.00 | 13.20 | DIC |
| ATOM | 1309 | O | HIS | 167 | 34.673 | 30.410 | 74.600 | 1.00 | 14.22 | DIC |
| ATOM | 1310 | N | ASP | 168 | 32.894 | 29.831 | 75.848 | 1.00 | 10.69 | DIC |
| ATOM | 1311 | CA | ASP | 168 | 32.045 | 29.572 | 74.699 | 1.00 | 10.18 | DIC |
| ATOM | 1312 | CB | ASP | 168 | 31.212 | 28.303 | 74.914 | 1.00 | 11.40 | DIC |
| ATOM | 1313 | CG | ASP | 168 | 30.499 | 27.860 | 73.654 | 1.00 | 11.10 | DIC |
| ATOM | 1314 | OD1 | ASP | 168 | 30.114 | 26.676 | 73.566 | 1.00 | 13.98 | DIC |
| ATOM | 1315 | OD2 | ASP | 168 | 30.317 | 28.698 | 72.748 | 1.00 | 13.04 | DIC |
| ATOM | 1316 | C | ASP | 168 | 31.150 | 30.801 | 74.568 | 1.00 | 9.57 | DIC |
| ATOM | 1317 | O | ASP | 168 | 30.019 | 30.830 | 75.056 | 1.00 | 6.68 | DIC |
| ATOM | 1318 | N | ILE | 169 | 31.690 | 31.818 | 73.904 | 1.00 | 8.34 | DIC |
| ATOM | 1319 | CA | ILE | 169 | 31.001 | 33.084 | 73.710 | 1.00 | 8.76 | DIC |
| ATOM | 1320 | CB | ILE | 169 | 31.918 | 34.092 | 72.985 | 1.00 | 8.29 | DIC |
| ATOM | 1321 | CG2 | ILE | 169 | 31.221 | 35.441 | 72.863 | 1.00 | 8.00 | DIC |
| ATOM | 1322 | CG1 | ILE | 169 | 33.230 | 34.244 | 73.759 | 1.00 | 10.02 | DIC |
| ATOM | 1323 | CD | ILE | 169 | 33.046 | 34.721 | 75.202 | 1.00 | 11.51 | DIC |
| ATOM | 1324 | C | ILE | 169 | 29.689 | 32.963 | 72.947 | 1.00 | 7.31 | DIC |
| ATOM | 1325 | O | ILE | 169 | 28.718 | 33.644 | 73.270 | 1.00 | 7.10 | DIC |
| ATOM | 1326 | N | PHE | 170 | 29.653 | 32.102 | 71.937 | 1.00 | 9.62 | DIC |
| ATOM | 1327 | CA | PHE | 170 | 28.428 | 31.936 | 71.162 | 1.00 | 9.67 | DIC |
| ATOM | 1328 | CB | PHE | 170 | 28.670 | 31.053 | 69.941 | 1.00 | 11.96 | DIC |
| ATOM | 1329 | CG | PHE | 170 | 27.451 | 30.875 | 69.082 | 1.00 | 12.14 | DIC |
| ATOM | 1330 | CD1 | PHE | 170 | 26.782 | 29.657 | 69.043 | 1.00 | 15.39 | DIC |
| ATOM | 1331 | CD2 | PHE | 170 | 26.950 | 31.939 | 68.339 | 1.00 | 13.78 | DIC |
| ATOM | 1332 | CE1 | PHE | 170 | 25.628 | 29.500 | 68.276 | 1.00 | 16.66 | DIC |
| ATOM | 1333 | CE2 | PHE | 170 | 25.800 | 31.793 | 67.569 | 1.00 | 16.17 | DIC |
| ATOM | 1334 | CZ | PHE | 170 | 25.136 | 30.569 | 67.539 | 1.00 | 15.75 | DIC |
| ATOM | 1335 | C | PHE | 170 | 27.314 | 31.336 | 72.012 | 1.00 | 10.61 | DIC |
| ATOM | 1336 | O | PHE | 170 | 26.163 | 31.765 | 71.933 | 1.00 | 10.59 | DIC |
| ATOM | 1337 | N | LYS | 171 | 27.652 | 30.345 | 72.830 | 1.00 | 10.05 | DIC |
| ATOM | 1338 | CA | LYS | 171 | 26.648 | 29.723 | 73.682 | 1.00 | 10.03 | DIC |
| ATOM | 1339 | CB | LYS | 171 | 27.239 | 28.505 | 74.399 | 1.00 | 13.97 | DIC |
| ATOM | 1340 | CG | LYS | 171 | 26.201 | 27.670 | 75.138 | 1.00 | 18.06 | DIC |
| ATOM | 1341 | CD | LYS | 171 | 26.735 | 26.285 | 75.464 | 1.00 | 22.03 | DIC |
| ATOM | 1342 | CE | LYS | 171 | 27.078 | 25.516 | 74.194 | 1.00 | 24.58 | DIC |
| ATOM | 1343 | NZ | LYS | 171 | 27.566 | 24.142 | 74.489 | 1.00 | 29.13 | DIC |
| ATOM | 1344 | C | LYS | 171 | 26.137 | 30.743 | 74.699 | 1.00 | 9.12 | DIC |
| ATOM | 1345 | O | LYS | 171 | 24.935 | 30.853 | 74.928 | 1.00 | 9.26 | DIC |
| ATOM | 1346 | N | MSE | 172 | 27.058 | 31.485 | 75.303 | 1.00 | 6.97 | DIC |
| ATOM | 1347 | CA | MSE | 172 | 26.708 | 32.512 | 76.278 | 1.00 | 9.01 | DIC |
| ATOM | 1348 | CB | MSE | 172 | 27.980 | 33.168 | 76.828 | 1.00 | 11.24 | DIC |
| ATOM | 1349 | CG | MSE | 172 | 28.846 | 32.238 | 77.665 | 1.00 | 13.80 | DIC |
| ATOM | 1350 | SE | MSE | 172 | 30.625 | 32.946 | 77.970 | 1.00 | 25.22 | DIC |
| ATOM | 1351 | CE | MSE | 172 | 30.177 | 34.354 | 79.221 | 1.00 | 18.63 | DIC |
| ATOM | 1352 | C | MSE | 172 | 25.836 | 33.562 | 75.593 | 1.00 | 9.14 | DIC |
| ATOM | 1353 | O | MSE | 172 | 24.924 | 34.123 | 76.202 | 1.00 | 10.30 | DIC |
| ATOM | 1354 | N | SER | 173 | 26.126 | 33.819 | 74.320 | 1.00 | 7.61 | DIC |
| ATOM | 1355 | CA | SER | 173 | 25.364 | 34.790 | 73.543 | 1.00 | 8.52 | DIC |
| ATOM | 1356 | CB | SER | 173 | 26.038 | 35.042 | 72.191 | 1.00 | 8.17 | DIC |
| ATOM | 1357 | OG | SER | 173 | 27.276 | 35.717 | 72.350 | 1.00 | 7.46 | DIC |
| ATOM | 1358 | C | SER | 173 | 23.945 | 34.278 | 73.316 | 1.00 | 9.07 | DIC |
| ATOM | 1359 | O | SER | 173 | 22.972 | 35.020 | 73.482 | 1.00 | 7.42 | DIC |
| ATOM | 1360 | N | SER | 174 | 23.837 | 33.008 | 72.935 | 1.00 | 7.76 | DIC |
| ATOM | 1361 | CA | SER | 174 | 22.539 | 32.397 | 72.682 | 1.00 | 9.45 | DIC |
| ATOM | 1362 | CB | SER | 174 | 22.718 | 30.952 | 72.210 | 1.00 | 9.22 | DIC |
| ATOM | 1363 | OG | SER | 174 | 23.382 | 30.917 | 70.961 | 1.00 | 8.11 | DIC |
| ATOM | 1364 | C | SER | 174 | 21.651 | 32.428 | 73.916 | 1.00 | 9.49 | DIC |
| ATOM | 1365 | O | SER | 174 | 20.460 | 32.725 | 73.824 | 1.00 | 9.51 | DIC |
| ATOM | 1366 | N | GLU | 175 | 22.234 | 32.123 | 75.071 | 1.00 | 10.06 | DIC |
| ATOM | 1367 | CA | GLU | 175 | 21.486 | 32.118 | 76.321 | 1.00 | 11.19 | DIC |
| ATOM | 1368 | CB | GLU | 175 | 22.336 | 31.537 | 77.452 | 1.00 | 14.11 | DIC |
| ATOM | 1369 | CG | GLU | 175 | 22.905 | 30.162 | 77.158 | 1.00 | 18.91 | DIC |
| ATOM | 1370 | CD | GLU | 175 | 23.568 | 29.540 | 78.372 | 1.00 | 21.75 | DIC |
| ATOM | 1371 | OE1 | GLU | 175 | 24.311 | 30.255 | 79.079 | 1.00 | 21.00 | DIC |
| ATOM | 1372 | OE2 | GLU | 175 | 23.350 | 28.333 | 78.615 | 1.00 | 24.48 | DIC |
| ATOM | 1373 | C | GLU | 175 | 21.046 | 33.525 | 76.697 | 1.00 | 10.40 | DIC |
| ATOM | 1374 | O | GLU | 175 | 19.942 | 33.725 | 77.202 | 1.00 | 10.43 | DIC |
| ATOM | 1375 | N | TRP | 176 | 21.912 | 34.502 | 76.453 | 1.00 | 11.21 | DIC |
| ATOM | 1376 | CA | TRP | 176 | 21.588 | 35.886 | 76.778 | 1.00 | 10.92 | DIC |
| ATOM | 1377 | CB | TRP | 176 | 22.804 | 36.788 | 76.550 | 1.00 | 11.86 | DIC |
| ATOM | 1378 | CG | TRP | 176 | 22.661 | 38.151 | 77.172 | 1.00 | 10.34 | DIC |
| ATOM | 1379 | CD2 | TRP | 176 | 22.094 | 39.317 | 76.564 | 1.00 | 10.13 | DIC |
| ATOM | 1380 | CE2 | TRP | 176 | 22.131 | 40.350 | 77.525 | 1.00 | 10.66 | DIC |
| ATOM | 1381 | CE3 | TRP | 176 | 21.555 | 39.588 | 75.299 | 1.00 | 11.84 | DIC |
| ATOM | 1382 | CD1 | TRP | 176 | 23.009 | 38.513 | 78.440 | 1.00 | 10.51 | DIC |
| ATOM | 1383 | NE1 | TRP | 176 | 22.695 | 39.832 | 78.662 | 1.00 | 11.39 | DIC |
| ATOM | 1384 | CZ2 | TRP | 176 | 21.649 | 41.636 | 77.263 | 1.00 | 10.57 | DIC |
| ATOM | 1385 | CZ3 | TRP | 176 | 21.073 | 40.869 | 75.037 | 1.00 | 9.09 | DIC |
| ATOM | 1386 | CH2 | TRP | 176 | 21.124 | 41.876 | 76.016 | 1.00 | 10.83 | DIC |
| ATOM | 1387 | C | TRP | 176 | 20.422 | 36.351 | 75.903 | 1.00 | 10.57 | DIC |
| ATOM | 1388 | O | TRP | 176 | 19.496 | 37.008 | 76.386 | 1.00 | 10.92 | DIC |
| ATOM | 1389 | N | VAL | 177 | 20.474 | 36.008 | 74.617 | 1.00 | 9.67 | DIC |
| ATOM | 1390 | CA | VAL | 177 | 19.415 | 36.382 | 73.681 | 1.00 | 8.71 | DIC |
| ATOM | 1391 | CB | VAL | 177 | 19.737 | 35.905 | 72.245 | 1.00 | 10.47 | DIC |
| ATOM | 1392 | CG1 | VAL | 177 | 18.483 | 35.975 | 71.369 | 1.00 | 9.48 | DIC |
| ATOM | 1393 | CG2 | VAL | 177 | 20.836 | 36.771 | 71.642 | 1.00 | 5.59 | DIC |
| ATOM | 1394 | C | VAL | 177 | 18.091 | 35.758 | 74.118 | 1.00 | 10.57 | DIC |
| ATOM | 1395 | O | VAL | 177 | 17.027 | 36.376 | 74.019 | 1.00 | 9.35 | DIC |
| ATOM | 1396 | N | GLU | 178 | 18.160 | 34.524 | 74.597 | 1.00 | 11.52 | DIC |
| ATOM | 1397 | CA | GLU | 178 | 16.961 | 33.833 | 75.038 | 1.00 | 15.37 | DIC |
| ATOM | 1398 | CB | GLU | 178 | 17.310 | 32.407 | 75.471 | 1.00 | 16.80 | DIC |
| ATOM | 1399 | CG | GLU | 178 | 16.111 | 31.557 | 75.857 | 1.00 | 22.43 | DIC |
| ATOM | 1400 | CD | GLU | 178 | 15.023 | 31.554 | 74.797 | 1.00 | 25.01 | DIC |
| ATOM | 1401 | OE1 | GLU | 178 | 15.345 | 31.366 | 73.603 | 1.00 | 27.14 | DIC |
| ATOM | 1402 | OE2 | GLU | 178 | 13.841 | 31.735 | 75.161 | 1.00 | 29.11 | DIC |
| ATOM | 1403 | C | GLU | 178 | 16.355 | 34.603 | 76.204 | 1.00 | 15.38 | DIC |
| ATOM | 1404 | O | GLU | 178 | 15.139 | 34.679 | 76.352 | 1.00 | 15.87 | DIC |
| ATOM | 1405 | N | GLN | 179 | 17.223 | 35.198 | 77.012 | 1.00 | 15.89 | DIC |
| ATOM | 1406 | CA | GLN | 179 | 16.803 | 35.954 | 78.180 | 1.00 | 17.35 | DIC |
| ATOM | 1407 | CB | GLN | 179 | 17.962 | 35.998 | 79.182 | 1.00 | 19.05 | DIC |
| ATOM | 1408 | CG | GLN | 179 | 17.658 | 36.694 | 80.493 | 1.00 | 24.86 | DIC |
| ATOM | 1409 | CD | GLN | 179 | 18.839 | 36.659 | 81.454 | 1.00 | 27.98 | DIC |
| ATOM | 1410 | OE1 | GLN | 179 | 18.761 | 37.170 | 82.573 | 1.00 | 31.11 | DIC |
| ATOM | 1411 | NE2 | GLN | 179 | 19.939 | 36.053 | 81.019 | 1.00 | 29.53 | DIC |
| ATOM | 1412 | C | GLN | 179 | 16.313 | 37.375 | 77.884 | 1.00 | 16.67 | DIC |
| ATOM | 1413 | O | GLN | 179 | 15.381 | 37.850 | 78.528 | 1.00 | 17.16 | DIC |
| ATOM | 1414 | N | TYR | 180 | 16.918 | 38.045 | 76.904 | 1.00 | 15.88 | DIC |
| ATOM | 1415 | CA | TYR | 180 | 16.543 | 39.423 | 76.585 | 1.00 | 14.53 | DIC |
| ATOM | 1416 | CB | TYR | 180 | 17.736 | 40.347 | 76.831 | 1.00 | 15.01 | DIC |
| ATOM | 1417 | CG | TYR | 180 | 18.196 | 40.402 | 78.264 | 1.00 | 12.97 | DIC |
| ATOM | 1418 | CD1 | TYR | 180 | 19.108 | 39.476 | 78.765 | 1.00 | 13.79 | DIC |
| ATOM | 1419 | CE1 | TYR | 180 | 19.545 | 39.543 | 80.085 | 1.00 | 15.48 | DIC |
| ATOM | 1420 | CD2 | TYR | 180 | 17.725 | 41.393 | 79.121 | 1.00 | 14.79 | DIC |
| ATOM | 1421 | CE2 | TYR | 180 | 18.152 | 41.466 | 80.439 | 1.00 | 15.22 | DIC |
| ATOM | 1422 | CZ | TYR | 180 | 19.062 | 40.542 | 80.915 | 1.00 | 14.58 | DIC |
| ATOM | 1423 | OH | TYR | 180 | 19.489 | 40.624 | 82.222 | 1.00 | 18.45 | DIC |
| ATOM | 1424 | C | TYR | 180 | 16.011 | 39.696 | 75.177 | 1.00 | 15.69 | DIC |
| ATOM | 1425 | O | TYR | 180 | 15.974 | 40.846 | 74.741 | 1.00 | 14.37 | DIC |
| ATOM | 1426 | N | LYS | 181 | 15.597 | 38.650 | 74.474 | 1.00 | 15.60 | DIC |
| ATOM | 1427 | CA | LYS | 181 | 15.082 | 38.788 | 73.116 | 1.00 | 18.04 | DIC |
| ATOM | 1428 | CB | LYS | 181 | 14.660 | 37.415 | 72.585 | 1.00 | 19.59 | DIC |
| ATOM | 1429 | CG | LYS | 181 | 13.984 | 37.447 | 71.226 | 1.00 | 24.70 | DIC |
| ATOM | 1430 | CD | LYS | 181 | 14.910 | 37.984 | 70.145 | 1.00 | 27.36 | DIC |
| ATOM | 1431 | CE | LYS | 181 | 14.215 | 37.989 | 68.789 | 1.00 | 27.65 | DIC |
| ATOM | 1432 | NZ | LYS | 181 | 13.752 | 36.628 | 68.397 | 1.00 | 29.00 | DIC |
| ATOM | 1433 | C | LYS | 181 | 13.910 | 39.762 | 72.973 | 1.00 | 16.82 | DIC |
| ATOM | 1434 | O | LYS | 181 | 13.759 | 40.414 | 71.939 | 1.00 | 16.53 | DIC |
| ATOM | 1435 | N | ASP | 182 | 13.083 | 39.860 | 74.006 | 1.00 | 16.61 | DIC |
| ATOM | 1436 | CA | ASP | 182 | 11.915 | 40.738 | 73.964 | 1.00 | 18.34 | DIC |
| ATOM | 1437 | CB | ASP | 182 | 10.842 | 40.220 | 74.920 | 1.00 | 19.12 | DIC |
| ATOM | 1438 | CG | ASP | 182 | 10.268 | 38.885 | 74.492 | 1.00 | 20.80 | DIC |
| ATOM | 1439 | OD1 | ASP | 182 | 9.625 | 38.232 | 75.336 | 1.00 | 23.00 | DIC |
| ATOM | 1440 | OD2 | ASP | 182 | 10.447 | 38.491 | 73.317 | 1.00 | 23.17 | DIC |
| ATOM | 1441 | C | ASP | 182 | 12.197 | 42.199 | 74.301 | 1.00 | 17.72 | DIC |
| ATOM | 1442 | O | ASP | 182 | 11.312 | 43.042 | 74.178 | 1.00 | 19.56 | DIC |
| ATOM | 1443 | N | VAL | 183 | 13.416 | 42.507 | 74.724 | 1.00 | 16.43 | DIC |
| ATOM | 1444 | CA | VAL | 183 | 13.737 | 43.880 | 75.092 | 1.00 | 15.35 | DIC |
| ATOM | 1445 | CB | VAL | 183 | 14.003 | 43.979 | 76.619 | 1.00 | 17.44 | DIC |
| ATOM | 1446 | CG1 | VAL | 183 | 15.207 | 43.133 | 76.998 | 1.00 | 17.05 | DIC |
| ATOM | 1447 | CG2 | VAL | 183 | 14.215 | 45.422 | 77.022 | 1.00 | 21.13 | DIC |
| ATOM | 1448 | C | VAL | 183 | 14.914 | 44.489 | 74.334 | 1.00 | 14.50 | DIC |
| ATOM | 1449 | O | VAL | 183 | 14.957 | 45.703 | 74.122 | 1.00 | 13.37 | DIC |
| ATOM | 1450 | N | MSE | 184 | 15.863 | 43.659 | 73.914 | 1.00 | 13.04 | DIC |
| ATOM | 1451 | CA | MSE | 184 | 17.024 | 44.177 | 73.203 | 1.00 | 12.36 | DIC |
| ATOM | 1452 | CB | MSE | 184 | 18.002 | 43.038 | 72.852 | 1.00 | 15.98 | DIC |
| ATOM | 1453 | CG | MSE | 184 | 17.427 | 41.874 | 72.058 | 1.00 | 17.28 | DIC |
| ATOM | 1454 | SE | MSE | 184 | 18.615 | 40.314 | 72.140 | 1.00 | 23.65 | DIC |
| ATOM | 1455 | CE | MSE | 184 | 19.958 | 40.898 | 70.874 | 1.00 | 16.42 | DIC |
| ATOM | 1456 | C | MSE | 184 | 16.641 | 44.976 | 71.960 | 1.00 | 11.92 | DIC |
| ATOM | 1457 | O | MSE | 184 | 15.749 | 44.594 | 71.198 | 1.00 | 11.41 | DIC |
| ATOM | 1458 | N | GLN | 185 | 17.324 | 46.098 | 71.772 | 1.00 | 10.32 | DIC |
| ATOM | 1459 | CA | GLN | 185 | 17.066 | 46.983 | 70.647 | 1.00 | 10.61 | DIC |
| ATOM | 1460 | CB | GLN | 185 | 16.644 | 48.362 | 71.164 | 1.00 | 13.31 | DIC |
| ATOM | 1461 | CG | GLN | 185 | 15.271 | 48.406 | 71.823 | 1.00 | 13.72 | DIC |
| ATOM | 1462 | CD | GLN | 185 | 14.959 | 49.766 | 72.438 | 1.00 | 16.61 | DIC |
| ATOM | 1463 | OE1 | GLN | 185 | 15.407 | 50.805 | 71.944 | 1.00 | 14.88 | DIC |
| ATOM | 1464 | NE2 | GLN | 185 | 14.174 | 49.764 | 73.513 | 1.00 | 14.58 | DIC |
| ATOM | 1465 | C | GLN | 185 | 18.287 | 47.141 | 69.748 | 1.00 | 9.38 | DIC |
| ATOM | 1466 | O | GLN | 185 | 18.167 | 47.581 | 68.602 | 1.00 | 7.58 | DIC |
| ATOM | 1467 | N | TYR | 186 | 19.460 | 46.772 | 70.254 | 1.00 | 7.31 | DIC |
| ATOM | 1468 | CA | TYR | 186 | 20.670 | 46.938 | 69.465 | 1.00 | 8.32 | DIC |
| ATOM | 1469 | CB | TYR | 186 | 21.601 | 47.929 | 70.169 | 1.00 | 8.66 | DIC |
| ATOM | 1470 | CG | TYR | 186 | 20.939 | 49.274 | 70.383 | 1.00 | 12.08 | DIC |
| ATOM | 1471 | CD1 | TYR | 186 | 20.325 | 49.589 | 71.594 | 1.00 | 11.99 | DIC |
| ATOM | 1472 | CE1 | TYR | 186 | 19.659 | 50.805 | 71.774 | 1.00 | 11.92 | DIC |
| ATOM | 1473 | CD2 | TYR | 186 | 20.873 | 50.213 | 69.348 | 1.00 | 14.31 | DIC |
| ATOM | 1474 | CE2 | TYR | 186 | 20.210 | 51.428 | 69.516 | 1.00 | 13.76 | DIC |
| ATOM | 1475 | CZ | TYR | 186 | 19.605 | 51.716 | 70.732 | 1.00 | 13.90 | DIC |
| ATOM | 1476 | OH | TYR | 186 | 18.947 | 52.915 | 70.901 | 1.00 | 12.39 | DIC |
| ATOM | 1477 | C | TYR | 186 | 21.422 | 45.672 | 69.065 | 1.00 | 6.28 | DIC |
| ATOM | 1478 | O | TYR | 186 | 22.640 | 45.679 | 68.913 | 1.00 | 5.43 | DIC |
| ATOM | 1479 | N | GLN | 187 | 20.676 | 44.587 | 68.907 | 1.00 | 5.24 | DIC |
| ATOM | 1480 | CA | GLN | 187 | 21.217 | 43.316 | 68.445 | 1.00 | 5.09 | DIC |
| ATOM | 1481 | CB | GLN | 187 | 21.306 | 43.393 | 66.913 | 1.00 | 6.13 | DIC |
| ATOM | 1482 | CG | GLN | 187 | 20.085 | 44.111 | 66.309 | 1.00 | 6.96 | DIC |
| ATOM | 1483 | CD | GLN | 187 | 20.155 | 44.327 | 64.800 | 1.00 | 9.84 | DIC |
| ATOM | 1484 | OE1 | GLN | 187 | 21.221 | 44.577 | 64.235 | 1.00 | 11.83 | DIC |
| ATOM | 1485 | NE2 | GLN | 187 | 19.005 | 44.262 | 64.150 | 1.00 | 8.23 | DIC |
| ATOM | 1486 | C | GLN | 187 | 22.550 | 42.857 | 69.065 | 1.00 | 4.73 | DIC |
| ATOM | 1487 | O | GLN | 187 | 22.659 | 42.726 | 70.285 | 1.00 | 5.09 | DIC |
| ATOM | 1488 | N | ASP | 188 | 23.561 | 42.608 | 68.235 | 1.00 | 4.04 | DIC |
| ATOM | 1489 | CA | ASP | 188 | 24.849 | 42.128 | 68.740 | 1.00 | 4.79 | DIC |
| ATOM | 1490 | CB | ASP | 188 | 25.832 | 41.901 | 67.589 | 1.00 | 5.88 | DIC |
| ATOM | 1491 | CG | ASP | 188 | 26.115 | 43.164 | 66.807 | 1.00 | 7.65 | DIC |
| ATOM | 1492 | OD1 | ASP | 188 | 25.242 | 43.574 | 66.013 | 1.00 | 8.96 | DIC |
| ATOM | 1493 | OD2 | ASP | 188 | 27.203 | 43.747 | 66.990 | 1.00 | 5.04 | DIC |
| ATOM | 1494 | C | ASP | 188 | 25.493 | 43.049 | 69.770 | 1.00 | 4.86 | DIC |
| ATOM | 1495 | O | ASP | 188 | 26.208 | 42.595 | 70.671 | 1.00 | 3.28 | DIC |
| ATOM | 1496 | N | GLN | 189 | 25.236 | 44.342 | 69.633 | 1.00 | 4.17 | DIC |
| ATOM | 1497 | CA | GLN | 189 | 25.789 | 45.334 | 70.547 | 1.00 | 5.49 | DIC |
| ATOM | 1498 | CB | GLN | 189 | 25.421 | 46.730 | 70.050 | 1.00 | 4.71 | DIC |
| ATOM | 1499 | CG | GLN | 189 | 26.008 | 47.868 | 70.848 | 1.00 | 5.10 | DIC |
| ATOM | 1500 | CD | GLN | 189 | 25.851 | 49.189 | 70.117 | 1.00 | 6.21 | DIC |
| ATOM | 1501 | OE1 | GLN | 189 | 26.760 | 49.634 | 69.414 | 1.00 | 6.99 | DIC |
| ATOM | 1502 | NE2 | GLN | 189 | 24.684 | 49.809 | 70.258 | 1.00 | 5.59 | DIC |
| ATOM | 1503 | C | GLN | 189 | 25.258 | 45.101 | 71.963 | 1.00 | 5.73 | DIC |
| ATOM | 1504 | O | GLN | 189 | 25.990 | 45.232 | 72.940 | 1.00 | 5.04 | DIC |
| ATOM | 1505 | N | ASP | 190 | 23.980 | 44.755 | 72.064 | 1.00 | 6.60 | DIC |
| ATOM | 1506 | CA | ASP | 190 | 23.371 | 44.480 | 73.361 | 1.00 | 6.99 | DIC |
| ATOM | 1507 | CB | ASP | 190 | 21.873 | 44.203 | 73.200 | 1.00 | 6.82 | DIC |
| ATOM | 1508 | CG | ASP | 190 | 21.061 | 45.466 | 72.999 | 1.00 | 7.01 | DIC |
| ATOM | 1509 | OD1 | ASP | 190 | 19.957 | 45.369 | 72.422 | 1.00 | 5.87 | DIC |
| ATOM | 1510 | OD2 | ASP | 190 | 21.515 | 46.547 | 73.428 | 1.00 | 5.97 | DIC |
| ATOM | 1511 | C | ASP | 190 | 24.040 | 43.256 | 73.976 | 1.00 | 6.81 | DIC |
| ATOM | 1512 | O | ASP | 190 | 24.454 | 43.277 | 75.135 | 1.00 | 8.46 | DIC |
| ATOM | 1513 | N | ILE | 191 | 24.135 | 42.188 | 73.187 | 1.00 | 6.27 | DIC |
| ATOM | 1514 | CA | ILE | 191 | 24.747 | 40.947 | 73.645 | 1.00 | 5.03 | DIC |
| ATOM | 1515 | CB | ILE | 191 | 24.896 | 39.932 | 72.487 | 1.00 | 3.31 | DIC |
| ATOM | 1516 | CG2 | ILE | 191 | 25.566 | 38.655 | 72.999 | 1.00 | 1.40 | DIC |
| ATOM | 1517 | CG1 | ILE | 191 | 23.518 | 39.598 | 71.909 | 1.00 | 4.10 | DIC |
| ATOM | 1518 | CD | ILE | 191 | 23.555 | 38.693 | 70.687 | 1.00 | 1.67 | DIC |
| ATOM | 1519 | C | ILE | 191 | 26.118 | 41.188 | 74.259 | 1.00 | 5.12 | DIC |
| ATOM | 1520 | O | ILE | 191 | 26.389 | 40.751 | 75.376 | 1.00 | 6.29 | DIC |
| ATOM | 1521 | N | LEU | 192 | 26.979 | 41.890 | 73.529 | 1.00 | 4.92 | DIC |
| ATOM | 1522 | CA | LEU | 192 | 28.326 | 42.168 | 74.009 | 1.00 | 5.86 | DIC |
| ATOM | 1523 | CB | LEU | 192 | 29.107 | 42.956 | 72.955 | 1.00 | 6.75 | DIC |
| ATOM | 1524 | CG | LEU | 192 | 29.278 | 42.257 | 71.601 | 1.00 | 6.62 | DIC |
| ATOM | 1525 | CD1 | LEU | 192 | 29.967 | 43.202 | 70.626 | 1.00 | 6.82 | DIC |
| ATOM | 1526 | CD2 | LEU | 192 | 30.086 | 40.988 | 71.771 | 1.00 | 8.14 | DIC |
| ATOM | 1527 | C | LEU | 192 | 28.306 | 42.930 | 75.332 | 1.00 | 6.87 | DIC |
| ATOM | 1528 | O | LEU | 192 | 29.053 | 42.603 | 76.253 | 1.00 | 6.68 | DIC |
| ATOM | 1529 | N | ASN | 193 | 27.449 | 43.943 | 75.424 | 1.00 | 6.22 | DIC |
| ATOM | 1530 | CA | ASN | 193 | 27.339 | 44.732 | 76.650 | 1.00 | 8.19 | DIC |
| ATOM | 1531 | CB | ASN | 193 | 26.403 | 45.921 | 76.434 | 1.00 | 8.71 | DIC |
| ATOM | 1532 | CG | ASN | 193 | 27.139 | 47.159 | 75.972 | 1.00 | 9.44 | DIC |
| ATOM | 1533 | OD1 | ASN | 193 | 27.826 | 47.814 | 76.760 | 1.00 | 7.46 | DIC |
| ATOM | 1534 | ND2 | ASN | 193 | 27.007 | 47.487 | 74.690 | 1.00 | 6.34 | DIC |
| ATOM | 1535 | C | ASN | 193 | 26.827 | 43.897 | 77.815 | 1.00 | 7.45 | DIC |
| ATOM | 1536 | O | ASN | 193 | 27.355 | 43.977 | 78.926 | 1.00 | 9.83 | DIC |
| ATOM | 1537 | N | GLY | 194 | 25.798 | 43.099 | 77.556 | 1.00 | 7.11 | DIC |
| ATOM | 1538 | CA | GLY | 194 | 25.227 | 42.268 | 78.599 | 1.00 | 7.61 | DIC |
| ATOM | 1539 | C | GLY | 194 | 26.196 | 41.240 | 79.139 | 1.00 | 8.02 | DIC |
| ATOM | 1540 | O | GLY | 194 | 26.318 | 41.064 | 80.351 | 1.00 | 8.13 | DIC |
| ATOM | 1541 | N | LEU | 195 | 26.893 | 40.559 | 78.237 | 1.00 | 6.93 | DIC |
| ATOM | 1542 | CA | LEU | 195 | 27.851 | 39.539 | 78.632 | 1.00 | 8.13 | DIC |
| ATOM | 1543 | CB | LEU | 195 | 28.330 | 38.746 | 77.407 | 1.00 | 5.03 | DIC |
| ATOM | 1544 | CG | LEU | 195 | 27.361 | 37.849 | 76.641 | 1.00 | 6.16 | DIC |
| ATOM | 1545 | CD1 | LEU | 195 | 28.134 | 37.104 | 75.553 | 1.00 | 5.85 | DIC |
| ATOM | 1546 | CD2 | LEU | 195 | 26.693 | 36.861 | 77.591 | 1.00 | 6.84 | DIC |
| ATOM | 1547 | C | LEU | 195 | 29.086 | 40.077 | 79.349 | 1.00 | 7.32 | DIC |
| ATOM | 1548 | O | LEU | 195 | 29.555 | 39.478 | 80.314 | 1.00 | 6.97 | DIC |
| ATOM | 1549 | N | PHE | 196 | 29.609 | 41.205 | 78.882 | 1.00 | 8.32 | DIC |
| ATOM | 1550 | CA | PHE | 196 | 30.842 | 41.739 | 79.448 | 1.00 | 6.43 | DIC |
| ATOM | 1551 | CB | PHE | 196 | 31.788 | 42.122 | 78.302 | 1.00 | 7.48 | DIC |
| ATOM | 1552 | CG | PHE | 196 | 31.994 | 41.021 | 77.293 | 1.00 | 7.64 | DIC |
| ATOM | 1553 | CD1 | PHE | 196 | 32.079 | 39.694 | 77.702 | 1.00 | 7.32 | DIC |
| ATOM | 1554 | CD2 | PHE | 196 | 32.138 | 41.315 | 75.939 | 1.00 | 8.39 | DIC |
| ATOM | 1555 | CE1 | PHE | 196 | 32.309 | 38.669 | 76.783 | 1.00 | 9.53 | DIC |
| ATOM | 1556 | CE2 | PHE | 196 | 32.370 | 40.300 | 75.008 | 1.00 | 8.57 | DIC |
| ATOM | 1557 | CZ | PHE | 196 | 32.456 | 38.974 | 75.431 | 1.00 | 9.27 | DIC |
| ATOM | 1558 | C | PHE | 196 | 30.773 | 42.883 | 80.453 | 1.00 | 7.23 | DIC |
| ATOM | 1559 | O | PHE | 196 | 31.813 | 43.342 | 80.931 | 1.00 | 6.97 | DIC |
| ATOM | 1560 | N | LYS | 197 | 29.571 | 43.340 | 80.787 | 1.00 | 7.17 | DIC |
| ATOM | 1561 | CA | LYS | 197 | 29.430 | 44.428 | 81.745 | 1.00 | 7.37 | DIC |
| ATOM | 1562 | CB | LYS | 197 | 27.950 | 44.632 | 82.106 | 1.00 | 8.11 | DIC |
| ATOM | 1563 | CG | LYS | 197 | 27.239 | 43.431 | 82.719 | 1.00 | 9.34 | DIC |
| ATOM | 1564 | CD | LYS | 197 | 25.752 | 43.730 | 82.867 | 1.00 | 7.17 | DIC |
| ATOM | 1565 | CE | LYS | 197 | 24.991 | 42.602 | 83.562 | 1.00 | 9.65 | DIC |
| ATOM | 1566 | NZ | LYS | 197 | 25.011 | 41.324 | 82.791 | 1.00 | 9.78 | DIC |
| ATOM | 1567 | C | LYS | 197 | 30.260 | 44.164 | 83.001 | 1.00 | 8.15 | DIC |
| ATOM | 1568 | O | LYS | 197 | 30.289 | 43.049 | 83.517 | 1.00 | 6.36 | DIC |
| ATOM | 1569 | N | GLY | 198 | 30.951 | 45.194 | 83.478 | 1.00 | 8.63 | DIC |
| ATOM | 1570 | CA | GLY | 198 | 31.781 | 45.043 | 84.660 | 1.00 | 9.48 | DIC |
| ATOM | 1571 | C | GLY | 198 | 33.170 | 44.550 | 84.307 | 1.00 | 11.16 | DIC |
| ATOM | 1572 | O | GLY | 198 | 34.065 | 44.534 | 85.151 | 1.00 | 11.56 | DIC |
| ATOM | 1573 | N | GLY | 199 | 33.353 | 44.147 | 83.052 | 1.00 | 9.98 | DIC |
| ATOM | 1574 | CA | GLY | 199 | 34.649 | 43.656 | 82.617 | 1.00 | 10.95 | DIC |
| ATOM | 1575 | C | GLY | 199 | 35.067 | 44.312 | 81.315 | 1.00 | 11.00 | DIC |
| ATOM | 1576 | O | GLY | 199 | 35.598 | 43.659 | 80.420 | 1.00 | 10.44 | DIC |
| ATOM | 1577 | N | VAL | 200 | 34.823 | 45.614 | 81.217 | 1.00 | 9.87 | DIC |
| ATOM | 1578 | CA | VAL | 200 | 35.159 | 46.377 | 80.022 | 1.00 | 10.31 | DIC |
| ATOM | 1579 | CB | VAL | 200 | 33.890 | 47.027 | 79.405 | 1.00 | 9.97 | DIC |
| ATOM | 1580 | CG1 | VAL | 200 | 34.265 | 47.857 | 78.186 | 1.00 | 13.00 | DIC |
| ATOM | 1581 | CG2 | VAL | 200 | 32.884 | 45.955 | 79.018 | 1.00 | 9.98 | DIC |
| ATOM | 1582 | C | VAL | 200 | 36.157 | 47.492 | 80.315 | 1.00 | 9.00 | DIC |
| ATOM | 1583 | O | VAL | 200 | 36.037 | 48.197 | 81.316 | 1.00 | 9.62 | DIC |
| ATOM | 1584 | N | CYS | 201 | 37.149 | 47.633 | 79.443 | 1.00 | 10.08 | DIC |
| ATOM | 1585 | CA | CYS | 201 | 38.137 | 48.696 | 79.561 | 1.00 | 11.93 | DIC |
| ATOM | 1586 | CB | CYS | 201 | 39.508 | 48.204 | 79.102 | 1.00 | 16.16 | DIC |
| ATOM | 1587 | SG | CYS | 201 | 40.754 | 49.497 | 78.999 | 1.00 | 22.80 | DIC |
| ATOM | 1588 | C | CYS | 201 | 37.624 | 49.778 | 78.615 | 1.00 | 12.32 | DIC |
| ATOM | 1589 | O | CYS | 201 | 37.383 | 49.504 | 77.438 | 1.00 | 11.10 | DIC |
| ATOM | 1590 | N | TYR | 202 | 37.434 | 50.998 | 79.110 | 1.00 | 11.44 | DIC |
| ATOM | 1591 | CA | TYR | 202 | 36.914 | 52.051 | 78.245 | 1.00 | 11.56 | DIC |
| ATOM | 1592 | CB | TYR | 202 | 36.008 | 53.001 | 79.034 | 1.00 | 13.47 | DIC |
| ATOM | 1593 | CG | TYR | 202 | 34.888 | 52.317 | 79.784 | 1.00 | 12.77 | DIC |
| ATOM | 1594 | CD1 | TYR | 202 | 34.977 | 52.112 | 81.159 | 1.00 | 14.55 | DIC |
| ATOM | 1595 | CE1 | TYR | 202 | 33.950 | 51.501 | 81.865 | 1.00 | 15.50 | DIC |
| ATOM | 1596 | CD2 | TYR | 202 | 33.734 | 51.884 | 79.124 | 1.00 | 13.35 | DIC |
| ATOM | 1597 | CE2 | TYR | 202 | 32.695 | 51.263 | 79.824 | 1.00 | 13.55 | DIC |
| ATOM | 1598 | CZ | TYR | 202 | 32.815 | 51.080 | 81.197 | 1.00 | 14.96 | DIC |
| ATOM | 1599 | OH | TYR | 202 | 31.801 | 50.494 | 81.912 | 1.00 | 15.14 | DIC |
| ATOM | 1600 | C | TYR | 202 | 37.998 | 52.860 | 77.543 | 1.00 | 12.95 | DIC |
| ATOM | 1601 | O | TYR | 202 | 38.970 | 53.291 | 78.160 | 1.00 | 12.67 | DIC |
| ATOM | 1602 | N | ALA | 203 | 37.818 | 53.060 | 76.243 | 1.00 | 12.15 | DIC |
| ATOM | 1603 | CA | ALA | 203 | 38.762 | 53.829 | 75.443 | 1.00 | 12.67 | DIC |
| ATOM | 1604 | CB | ALA | 203 | 39.001 | 53.134 | 74.107 | 1.00 | 11.34 | DIC |
| ATOM | 1605 | C | ALA | 203 | 38.181 | 55.220 | 75.212 | 1.00 | 11.62 | DIC |
| ATOM | 1606 | O | ALA | 203 | 36.968 | 55.412 | 75.300 | 1.00 | 10.59 | DIC |
| ATOM | 1607 | N | ASN | 204 | 39.041 | 56.188 | 74.916 | 1.00 | 10.55 | DIC |
| ATOM | 1608 | CA | ASN | 204 | 38.575 | 57.550 | 74.669 | 1.00 | 9.36 | DIC |
| ATOM | 1609 | CB | ASN | 204 | 39.758 | 58.498 | 74.487 | 1.00 | 7.28 | DIC |
| ATOM | 1610 | CG | ASN | 204 | 39.333 | 59.943 | 74.432 | 1.00 | 9.32 | DIC |
| ATOM | 1611 | OD1 | ASN | 204 | 39.037 | 60.559 | 75.463 | 1.00 | 9.40 | DIC |
| ATOM | 1612 | ND2 | ASN | 204 | 39.281 | 60.494 | 73.226 | 1.00 | 5.22 | DIC |
| ATOM | 1613 | C | ASN | 204 | 37.711 | 57.566 | 73.411 | 1.00 | 8.45 | DIC |
| ATOM | 1614 | O | ASN | 204 | 37.921 | 56.771 | 72.493 | 1.00 | 8.52 | DIC |
| ATOM | 1615 | N | SER | 205 | 36.743 | 58.474 | 73.370 | 1.00 | 6.68 | DIC |
| ATOM | 1616 | CA | SER | 205 | 35.845 | 58.573 | 72.227 | 1.00 | 8.43 | DIC |
| ATOM | 1617 | CB | SER | 205 | 34.708 | 59.552 | 72.539 | 1.00 | 6.55 | DIC |
| ATOM | 1618 | OG | SER | 205 | 33.769 | 58.945 | 73.415 | 1.00 | 5.21 | DIC |
| ATOM | 1619 | C | SER | 205 | 36.520 | 58.947 | 70.908 | 1.00 | 8.25 | DIC |
| ATOM | 1620 | O | SER | 205 | 35.890 | 58.884 | 69.846 | 1.00 | 7.41 | DIC |
| ATOM | 1621 | N | ARG | 206 | 37.796 | 59.324 | 70.959 | 1.00 | 6.24 | DIC |
| ATOM | 1622 | CA | ARG | 206 | 38.503 | 59.664 | 69.727 | 1.00 | 5.53 | DIC |
| ATOM | 1623 | CB | ARG | 206 | 39.901 | 60.235 | 70.031 | 1.00 | 5.02 | DIC |
| ATOM | 1624 | CG | ARG | 206 | 40.916 | 59.262 | 70.645 | 1.00 | 3.89 | DIC |
| ATOM | 1625 | CD | ARG | 206 | 42.220 | 60.002 | 71.003 | 1.00 | 6.14 | DIC |
| ATOM | 1626 | NE | ARG | 206 | 42.757 | 60.730 | 69.852 | 1.00 | 5.51 | DIC |
| ATOM | 1627 | CZ | ARG | 206 | 43.820 | 60.352 | 69.145 | 1.00 | 6.07 | DIC |
| ATOM | 1628 | NH1 | ARG | 206 | 44.486 | 59.252 | 69.471 | 1.00 | 5.54 | DIC |
| ATOM | 1629 | NH2 | ARG | 206 | 44.196 | 61.056 | 68.086 | 1.00 | 3.75 | DIC |
| ATOM | 1630 | C | ARG | 206 | 38.625 | 58.421 | 68.844 | 1.00 | 4.90 | DIC |
| ATOM | 1631 | O | ARG | 206 | 38.815 | 58.525 | 67.632 | 1.00 | 5.35 | DIC |
| ATOM | 1632 | N | PHE | 207 | 38.501 | 57.246 | 69.455 | 1.00 | 4.24 | DIC |
| ATOM | 1633 | CA | PHE | 207 | 38.612 | 55.988 | 68.722 | 1.00 | 4.90 | DIC |
| ATOM | 1634 | CB | PHE | 207 | 39.326 | 54.945 | 69.588 | 1.00 | 4.86 | DIC |
| ATOM | 1635 | CG | PHE | 207 | 40.764 | 55.288 | 69.875 | 1.00 | 5.99 | DIC |
| ATOM | 1636 | CD1 | PHE | 207 | 41.176 | 55.620 | 71.162 | 1.00 | 5.64 | DIC |
| ATOM | 1637 | CD2 | PHE | 207 | 41.700 | 55.322 | 68.842 | 1.00 | 4.91 | DIC |
| ATOM | 1638 | CE1 | PHE | 207 | 42.497 | 55.983 | 71.417 | 1.00 | 7.00 | DIC |
| ATOM | 1639 | CE2 | PHE | 207 | 43.024 | 55.684 | 69.085 | 1.00 | 7.26 | DIC |
| ATOM | 1640 | CZ | PHE | 207 | 43.424 | 56.016 | 70.375 | 1.00 | 6.13 | DIC |
| ATOM | 1641 | C | PHE | 207 | 37.276 | 55.439 | 68.224 | 1.00 | 5.37 | DIC |
| ATOM | 1642 | O | PHE | 207 | 37.208 | 54.338 | 67.682 | 1.00 | 6.61 | DIC |
| ATOM | 1643 | N | ASN | 208 | 36.215 | 56.214 | 68.414 | 1.00 | 5.44 | DIC |
| ATOM | 1644 | CA | ASN | 208 | 34.882 | 55.831 | 67.963 | 1.00 | 7.19 | DIC |
| ATOM | 1645 | CB | ASN | 208 | 34.279 | 54.752 | 68.863 | 1.00 | 4.53 | DIC |
| ATOM | 1646 | CG | ASN | 208 | 33.112 | 54.038 | 68.201 | 1.00 | 4.94 | DIC |
| ATOM | 1647 | OD1 | ASN | 208 | 32.464 | 54.587 | 67.304 | 1.00 | 3.79 | DIC |
| ATOM | 1648 | ND2 | ASN | 208 | 32.832 | 52.814 | 68.645 | 1.00 | 3.51 | DIC |
| ATOM | 1649 | C | ASN | 208 | 34.037 | 57.094 | 68.026 | 1.00 | 7.68 | DIC |
| ATOM | 1650 | O | ASN | 208 | 33.170 | 57.241 | 68.888 | 1.00 | 7.11 | DIC |
| ATOM | 1651 | N | PHE | 209 | 34.315 | 57.998 | 67.093 | 1.00 | 6.81 | DIC |
| ATOM | 1652 | CA | PHE | 209 | 33.647 | 59.288 | 67.004 | 1.00 | 8.34 | DIC |
| ATOM | 1653 | CB | PHE | 209 | 34.636 | 60.315 | 66.439 | 1.00 | 8.19 | DIC |
| ATOM | 1654 | CG | PHE | 209 | 34.226 | 61.740 | 66.648 | 1.00 | 7.78 | DIC |
| ATOM | 1655 | CD1 | PHE | 209 | 34.260 | 62.308 | 67.918 | 1.00 | 8.92 | DIC |
| ATOM | 1656 | CD2 | PHE | 209 | 33.830 | 62.525 | 65.569 | 1.00 | 9.53 | DIC |
| ATOM | 1657 | CE1 | PHE | 209 | 33.907 | 63.644 | 68.112 | 1.00 | 11.27 | DIC |
| ATOM | 1658 | CE2 | PHE | 209 | 33.476 | 63.858 | 65.751 | 1.00 | 8.49 | DIC |
| ATOM | 1659 | CZ | PHE | 209 | 33.515 | 64.419 | 67.025 | 1.00 | 9.96 | DIC |
| ATOM | 1660 | C | PHE | 209 | 32.431 | 59.166 | 66.094 | 1.00 | 8.23 | DIC |
| ATOM | 1661 | O | PHE | 209 | 32.569 | 59.073 | 64.877 | 1.00 | 8.27 | DIC |
| ATOM | 1662 | N | MSE | 210 | 31.244 | 59.179 | 66.692 | 1.00 | 7.38 | DIC |
| ATOM | 1663 | CA | MSE | 210 | 29.999 | 59.037 | 65.941 | 1.00 | 9.26 | DIC |
| ATOM | 1664 | CB | MSE | 210 | 29.159 | 57.918 | 66.581 | 1.00 | 9.05 | DIC |
| ATOM | 1665 | CG | MSE | 210 | 29.866 | 56.564 | 66.584 | 1.00 | 10.93 | DIC |
| ATOM | 1666 | SE | MSE | 210 | 29.082 | 55.239 | 67.779 | 1.00 | 18.26 | DIC |
| ATOM | 1667 | CE | MSE | 210 | 30.053 | 55.690 | 69.381 | 1.00 | 8.17 | DIC |
| ATOM | 1668 | C | MSE | 210 | 29.195 | 60.338 | 65.870 | 1.00 | 8.65 | DIC |
| ATOM | 1669 | O | MSE | 210 | 29.562 | 61.340 | 66.486 | 1.00 | 6.86 | DIC |
| ATOM | 1670 | N | PRO | 211 | 28.086 | 60.343 | 65.105 | 1.00 | 9.68 | DIC |
| ATOM | 1671 | CD | PRO | 211 | 27.573 | 59.289 | 64.211 | 1.00 | 9.09 | DIC |
| ATOM | 1672 | CA | PRO | 211 | 27.266 | 61.554 | 64.990 | 1.00 | 9.29 | DIC |
| ATOM | 1673 | CB | PRO | 211 | 26.037 | 61.058 | 64.240 | 1.00 | 9.60 | DIC |
| ATOM | 1674 | CG | PRO | 211 | 26.637 | 60.064 | 63.296 | 1.00 | 10.03 | DIC |
| ATOM | 1675 | C | PRO | 211 | 26.918 | 62.189 | 66.335 | 1.00 | 10.27 | DIC |
| ATOM | 1676 | O | PRO | 211 | 26.973 | 63.413 | 66.482 | 1.00 | 10.62 | DIC |
| ATOM | 1677 | N | THR | 212 | 26.572 | 61.367 | 67.322 | 1.00 | 7.87 | DIC |
| ATOM | 1678 | CA | THR | 212 | 26.222 | 61.906 | 68.629 | 1.00 | 8.03 | DIC |
| ATOM | 1679 | CB | THR | 212 | 25.663 | 60.807 | 69.561 | 1.00 | 8.27 | DIC |
| ATOM | 1680 | OG1 | THR | 212 | 24.528 | 60.191 | 68.939 | 1.00 | 9.40 | DIC |
| ATOM | 1681 | CG2 | THR | 212 | 25.216 | 61.410 | 70.886 | 1.00 | 9.47 | DIC |
| ATOM | 1682 | C | THR | 212 | 27.422 | 62.594 | 69.285 | 1.00 | 8.51 | DIC |
| ATOM | 1683 | O | THR | 212 | 27.266 | 63.607 | 69.975 | 1.00 | 8.07 | DIC |
| ATOM | 1684 | N | ASN | 213 | 28.616 | 62.046 | 69.074 | 1.00 | 6.60 | DIC |
| ATOM | 1685 | CA | ASN | 213 | 29.827 | 62.641 | 69.628 | 1.00 | 7.57 | DIC |
| ATOM | 1686 | CB | ASN | 213 | 31.047 | 61.768 | 69.315 | 1.00 | 7.05 | DIC |
| ATOM | 1687 | CG | ASN | 213 | 30.968 | 60.395 | 69.967 | 1.00 | 5.69 | DIC |
| ATOM | 1688 | OD1 | ASN | 213 | 31.421 | 60.198 | 71.098 | 1.00 | 5.17 | DIC |
| ATOM | 1689 | ND2 | ASN | 213 | 30.379 | 59.443 | 69.259 | 1.00 | 2.99 | DIC |
| ATOM | 1690 | C | ASN | 213 | 30.007 | 64.017 | 68.990 | 1.00 | 9.85 | DIC |
| ATOM | 1691 | O | ASN | 213 | 30.326 | 64.991 | 69.666 | 1.00 | 11.63 | DIC |
| ATOM | 1692 | N | TYR | 214 | 29.801 | 64.082 | 67.679 | 1.00 | 10.25 | DIC |
| ATOM | 1693 | CA | TYR | 214 | 29.926 | 65.333 | 66.945 | 1.00 | 12.74 | DIC |
| ATOM | 1694 | CB | TYR | 214 | 29.671 | 65.103 | 65.451 | 1.00 | 15.29 | DIC |
| ATOM | 1695 | CG | TYR | 214 | 29.730 | 66.365 | 64.619 | 1.00 | 17.71 | DIC |
| ATOM | 1696 | CD1 | TYR | 214 | 30.950 | 66.894 | 64.203 | 1.00 | 19.79 | DIC |
| ATOM | 1697 | CE1 | TYR | 214 | 31.011 | 68.072 | 63.461 | 1.00 | 21.10 | DIC |
| ATOM | 1698 | CD2 | TYR | 214 | 28.564 | 67.049 | 64.271 | 1.00 | 19.74 | DIC |
| ATOM | 1699 | CE2 | TYR | 214 | 28.612 | 68.230 | 63.530 | 1.00 | 22.09 | DIC |
| ATOM | 1700 | CZ | TYR | 214 | 29.838 | 68.735 | 63.128 | 1.00 | 22.87 | DIC |
| ATOM | 1701 | OH | TYR | 214 | 29.892 | 69.901 | 62.393 | 1.00 | 24.30 | DIC |
| ATOM | 1702 | C | TYR | 214 | 28.922 | 66.347 | 67.481 | 1.00 | 13.83 | DIC |
| ATOM | 1703 | O | TYR | 214 | 29.276 | 67.490 | 67.761 | 1.00 | 12.55 | DIC |
| ATOM | 1704 | N | ALA | 215 | 27.669 | 65.923 | 67.625 | 1.00 | 15.46 | DIC |
| ATOM | 1705 | CA | ALA | 215 | 26.615 | 66.803 | 68.124 | 1.00 | 17.53 | DIC |
| ATOM | 1706 | CB | ALA | 215 | 25.271 | 66.081 | 68.099 | 1.00 | 15.12 | DIC |
| ATOM | 1707 | C | ALA | 215 | 26.919 | 67.288 | 69.539 | 1.00 | 19.46 | DIC |
| ATOM | 1708 | O | ALA | 215 | 26.509 | 68.381 | 69.931 | 1.00 | 19.21 | DIC |
| ATOM | 1709 | N | ALA | 216 | 27.639 | 66.471 | 70.300 | 1.00 | 21.21 | DIC |
| ATOM | 1710 | CA | ALA | 216 | 27.991 | 66.821 | 71.669 | 1.00 | 24.59 | DIC |
| ATOM | 1711 | CB | ALA | 216 | 28.459 | 65.580 | 72.421 | 1.00 | 23.56 | DIC |
| ATOM | 1712 | C | ALA | 216 | 29.069 | 67.905 | 71.713 | 1.00 | 27.77 | DIC |
| ATOM | 1713 | O | ALA | 216 | 29.317 | 68.497 | 72.762 | 1.00 | 27.20 | DIC |
| ATOM | 1714 | N | MSE | 217 | 29.703 | 68.166 | 70.573 | 1.00 | 31.06 | DIC |
| ATOM | 1715 | CA | MSE | 217 | 30.743 | 69.189 | 70.498 | 1.00 | 34.21 | DIC |
| ATOM | 1716 | CB | MSE | 217 | 31.385 | 69.208 | 69.107 | 1.00 | 35.99 | DIC |
| ATOM | 1717 | CG | MSE | 217 | 32.193 | 67.966 | 68.760 | 1.00 | 39.28 | DIC |
| ATOM | 1718 | SE | MSE | 217 | 33.550 | 67.557 | 70.080 | 1.00 | 45.10 | DIC |
| ATOM | 1719 | CE | MSE | 217 | 34.867 | 68.888 | 69.607 | 1.00 | 42.38 | DIC |
| ATOM | 1720 | C | MSE | 217 | 30.175 | 70.570 | 70.809 | 1.00 | 35.31 | DIC |
| ATOM | 1721 | O | MSE | 217 | 29.016 | 70.860 | 70.503 | 1.00 | 36.62 | DIC |
| ATOM | 1722 | N | ALA | 222 | 25.134 | 68.364 | 77.425 | 1.00 | 31.46 | DIC |
| ATOM | 1723 | CA | ALA | 222 | 24.583 | 68.352 | 78.775 | 1.00 | 31.66 | DIC |
| ATOM | 1724 | CB | ALA | 222 | 23.312 | 67.513 | 78.813 | 1.00 | 32.95 | DIC |
| ATOM | 1725 | C | ALA | 222 | 25.604 | 67.803 | 79.765 | 1.00 | 31.68 | DIC |
| ATOM | 1726 | O | ALA | 222 | 26.479 | 68.538 | 80.229 | 1.00 | 32.14 | DIC |
| ATOM | 1727 | N | ALA | 223 | 25.483 | 66.511 | 80.071 | 1.00 | 31.10 | DIC |
| ATOM | 1728 | CA | ALA | 223 | 26.370 | 65.807 | 81.003 | 1.00 | 30.32 | DIC |
| ATOM | 1729 | CB | ALA | 223 | 26.806 | 66.741 | 82.137 | 1.00 | 30.79 | DIC |
| ATOM | 1730 | C | ALA | 223 | 25.626 | 64.605 | 81.581 | 1.00 | 28.04 | DIC |
| ATOM | 1731 | O | ALA | 223 | 24.856 | 64.749 | 82.528 | 1.00 | 29.66 | DIC |
| ATOM | 1732 | N | ALA | 224 | 25.856 | 63.422 | 81.018 | 1.00 | 27.00 | DIC |
| ATOM | 1733 | CA | ALA | 224 | 25.171 | 62.219 | 81.490 | 1.00 | 25.23 | DIC |
| ATOM | 1734 | CB | ALA | 224 | 25.440 | 61.057 | 80.543 | 1.00 | 25.60 | DIC |
| ATOM | 1735 | C | ALA | 224 | 25.530 | 61.817 | 82.918 | 1.00 | 24.60 | DIC |
| ATOM | 1736 | O | ALA | 224 | 24.642 | 61.593 | 83.742 | 1.00 | 24.76 | DIC |
| ATOM | 1737 | N | HIS | 225 | 26.825 | 61.716 | 83.206 | 1.00 | 23.10 | DIC |
| ATOM | 1738 | CA | HIS | 225 | 27.297 | 61.330 | 84.534 | 1.00 | 21.99 | DIC |
| ATOM | 1739 | CB | HIS | 225 | 27.907 | 59.926 | 84.490 | 1.00 | 22.53 | DIC |
| ATOM | 1740 | CG | HIS | 225 | 26.949 | 58.857 | 84.064 | 1.00 | 22.70 | DIC |
| ATOM | 1741 | CD2 | HIS | 225 | 26.829 | 58.183 | 82.896 | 1.00 | 21.83 | DIC |
| ATOM | 1742 | ND1 | HIS | 225 | 25.966 | 58.364 | 84.894 | 1.00 | 21.91 | DIC |
| ATOM | 1743 | CE1 | HIS | 225 | 25.282 | 57.430 | 84.256 | 1.00 | 22.15 | DIC |
| ATOM | 1744 | NE2 | HIS | 225 | 25.786 | 57.301 | 83.041 | 1.00 | 21.79 | DIC |
| ATOM | 1745 | C | HIS | 225 | 28.357 | 62.307 | 85.039 | 1.00 | 21.96 | DIC |
| ATOM | 1746 | O | HIS | 225 | 29.052 | 62.940 | 84.243 | 1.00 | 21.13 | DIC |
| ATOM | 1747 | N | THR | 226 | 28.480 | 62.422 | 86.360 | 1.00 | 21.29 | DIC |
| ATOM | 1748 | CA | THR | 226 | 29.474 | 63.313 | 86.956 | 1.00 | 20.93 | DIC |
| ATOM | 1749 | CB | THR | 226 | 29.032 | 63.827 | 88.347 | 1.00 | 20.27 | DIC |
| ATOM | 1750 | OG1 | THR | 226 | 28.812 | 62.715 | 89.225 | 1.00 | 23.22 | DIC |
| ATOM | 1751 | CG2 | THR | 226 | 27.756 | 64.637 | 88.233 | 1.00 | 21.25 | DIC |
| ATOM | 1752 | C | THR | 226 | 30.790 | 62.562 | 87.108 | 1.00 | 19.54 | DIC |
| ATOM | 1753 | O | THR | 226 | 31.854 | 63.168 | 87.205 | 1.00 | 19.24 | DIC |
| ATOM | 1754 | N | ASP | 227 | 30.702 | 61.236 | 87.133 | 1.00 | 18.16 | DIC |
| ATOM | 1755 | CA | ASP | 227 | 31.875 | 60.379 | 87.256 | 1.00 | 17.84 | DIC |
| ATOM | 1756 | CB | ASP | 227 | 31.512 | 58.958 | 86.827 | 1.00 | 19.84 | DIC |
| ATOM | 1757 | CG | ASP | 227 | 32.706 | 58.030 | 86.808 | 1.00 | 20.03 | DIC |
| ATOM | 1758 | OD1 | ASP | 227 | 32.607 | 56.955 | 86.185 | 1.00 | 21.19 | DIC |
| ATOM | 1759 | OD2 | ASP | 227 | 33.740 | 58.369 | 87.420 | 1.00 | 22.49 | DIC |
| ATOM | 1760 | C | ASP | 227 | 33.028 | 60.889 | 86.386 | 1.00 | 18.14 | DIC |
| ATOM | 1761 | O | ASP | 227 | 32.904 | 60.971 | 85.165 | 1.00 | 16.47 | DIC |
| ATOM | 1762 | N | PRO | 228 | 34.167 | 61.233 | 87.006 | 1.00 | 17.73 | DIC |
| ATOM | 1763 | CD | PRO | 228 | 34.480 | 61.151 | 88.443 | 1.00 | 18.17 | DIC |
| ATOM | 1764 | CA | PRO | 228 | 35.321 | 61.731 | 86.250 | 1.00 | 17.33 | DIC |
| ATOM | 1765 | CB | PRO | 228 | 36.363 | 61.995 | 87.339 | 1.00 | 18.94 | DIC |
| ATOM | 1766 | CG | PRO | 228 | 35.980 | 61.034 | 88.425 | 1.00 | 20.23 | DIC |
| ATOM | 1767 | C | PRO | 228 | 35.827 | 60.772 | 85.172 | 1.00 | 16.87 | DIC |
| ATOM | 1768 | O | PRO | 228 | 36.297 | 61.209 | 84.122 | 1.00 | 15.06 | DIC |
| ATOM | 1769 | N | LEU | 229 | 35.737 | 59.470 | 85.428 | 1.00 | 15.80 | DIC |
| ATOM | 1770 | CA | LEU | 229 | 36.188 | 58.496 | 84.446 | 1.00 | 15.66 | DIC |
| ATOM | 1771 | CB | LEU | 229 | 36.155 | 57.079 | 85.033 | 1.00 | 16.65 | DIC |
| ATOM | 1772 | CG | LEU | 229 | 36.728 | 55.969 | 84.147 | 1.00 | 17.28 | DIC |
| ATOM | 1773 | CD1 | LEU | 229 | 37.236 | 54.824 | 85.008 | 1.00 | 18.64 | DIC |
| ATOM | 1774 | CD2 | LEU | 229 | 35.661 | 55.487 | 83.172 | 1.00 | 15.76 | DIC |
| ATOM | 1775 | C | LEU | 229 | 35.281 | 58.596 | 83.224 | 1.00 | 14.21 | DIC |
| ATOM | 1776 | O | LEU | 229 | 35.759 | 58.658 | 82.092 | 1.00 | 15.12 | DIC |
| ATOM | 1777 | N | TYR | 230 | 33.972 | 58.628 | 83.457 | 1.00 | 14.01 | DIC |
| ATOM | 1778 | CA | TYR | 230 | 33.017 | 58.739 | 82.363 | 1.00 | 11.87 | DIC |
| ATOM | 1779 | CB | TYR | 230 | 31.585 | 58.846 | 82.895 | 1.00 | 14.20 | DIC |
| ATOM | 1780 | CG | TYR | 230 | 30.544 | 58.919 | 81.794 | 1.00 | 11.26 | DIC |
| ATOM | 1781 | CD1 | TYR | 230 | 30.146 | 57.774 | 81.105 | 1.00 | 13.91 | DIC |
| ATOM | 1782 | CE1 | TYR | 230 | 29.222 | 57.840 | 80.061 | 1.00 | 10.82 | DIC |
| ATOM | 1783 | CD2 | TYR | 230 | 29.991 | 60.139 | 81.414 | 1.00 | 12.75 | DIC |
| ATOM | 1784 | CE2 | TYR | 230 | 29.066 | 60.218 | 80.371 | 1.00 | 12.59 | DIC |
| ATOM | 1785 | CZ | TYR | 230 | 28.688 | 59.062 | 79.700 | 1.00 | 12.84 | DIC |
| ATOM | 1786 | OH | TYR | 230 | 27.779 | 59.132 | 78.665 | 1.00 | 11.30 | DIC |
| ATOM | 1787 | C | TYR | 230 | 33.332 | 59.983 | 81.538 | 1.00 | 11.96 | DIC |
| ATOM | 1788 | O | TYR | 230 | 33.441 | 59.913 | 80.317 | 1.00 | 9.30 | DIC |
| ATOM | 1789 | N | ARG | 231 | 33.472 | 61.119 | 82.218 | 1.00 | 11.98 | DIC |
| ATOM | 1790 | CA | ARG | 231 | 33.770 | 62.390 | 81.558 | 1.00 | 13.86 | DIC |
| ATOM | 1791 | CB | ARG | 231 | 33.823 | 63.515 | 82.598 | 1.00 | 16.38 | DIC |
| ATOM | 1792 | CG | ARG | 231 | 32.472 | 63.840 | 83.225 | 1.00 | 19.41 | DIC |
| ATOM | 1793 | CD | ARG | 231 | 32.635 | 64.503 | 84.588 | 1.00 | 24.54 | DIC |
| ATOM | 1794 | NE | ARG | 231 | 33.603 | 65.598 | 84.574 | 1.00 | 30.72 | DIC |
| ATOM | 1795 | CZ | ARG | 231 | 33.449 | 66.731 | 83.895 | 1.00 | 31.79 | DIC |
| ATOM | 1796 | NH1 | ARG | 231 | 34.390 | 67.667 | 83.945 | 1.00 | 33.17 | DIC |
| ATOM | 1797 | NH2 | ARG | 231 | 32.357 | 66.934 | 83.172 | 1.00 | 34.16 | DIC |
| ATOM | 1798 | C | ARG | 231 | 35.082 | 62.340 | 80.779 | 1.00 | 13.10 | DIC |
| ATOM | 1799 | O | ARG | 231 | 35.179 | 62.861 | 79.666 | 1.00 | 12.39 | DIC |
| ATOM | 1800 | N | ASP | 232 | 36.097 | 61.724 | 81.372 | 1.00 | 12.75 | DIC |
| ATOM | 1801 | CA | ASP | 232 | 37.393 | 61.609 | 80.716 | 1.00 | 12.99 | DIC |
| ATOM | 1802 | CB | ASP | 232 | 38.396 | 60.936 | 81.656 | 1.00 | 14.85 | DIC |
| ATOM | 1803 | CG | ASP | 232 | 39.709 | 60.610 | 80.973 | 1.00 | 16.86 | DIC |
| ATOM | 1804 | OD1 | ASP | 232 | 39.794 | 59.558 | 80.306 | 1.00 | 18.36 | DIC |
| ATOM | 1805 | OD2 | ASP | 232 | 40.656 | 61.412 | 81.094 | 1.00 | 16.54 | DIC |
| ATOM | 1806 | C | ASP | 232 | 37.302 | 60.819 | 79.406 | 1.00 | 12.82 | DIC |
| ATOM | 1807 | O | ASP | 232 | 37.864 | 61.224 | 78.387 | 1.00 | 10.29 | DIC |
| ATOM | 1808 | N | ARG | 233 | 36.584 | 59.699 | 79.435 | 1.00 | 10.29 | DIC |
| ATOM | 1809 | CA | ARG | 233 | 36.452 | 58.853 | 78.256 | 1.00 | 10.00 | DIC |
| ATOM | 1810 | CB | ARG | 233 | 35.932 | 57.470 | 78.661 | 1.00 | 9.23 | DIC |
| ATOM | 1811 | CG | ARG | 233 | 36.782 | 56.750 | 79.704 | 1.00 | 9.41 | DIC |
| ATOM | 1812 | CD | ARG | 233 | 38.153 | 56.364 | 79.163 | 1.00 | 8.38 | DIC |
| ATOM | 1813 | NE | ARG | 233 | 38.825 | 55.417 | 80.047 | 1.00 | 11.90 | DIC |
| ATOM | 1814 | CZ | ARG | 233 | 39.366 | 55.731 | 81.221 | 1.00 | 15.57 | DIC |
| ATOM | 1815 | NH1 | ARG | 233 | 39.332 | 56.983 | 81.666 | 1.00 | 14.61 | DIC |
| ATOM | 1816 | NH2 | ARG | 233 | 39.920 | 54.782 | 81.966 | 1.00 | 13.98 | DIC |
| ATOM | 1817 | C | ARG | 233 | 35.549 | 59.431 | 77.165 | 1.00 | 10.24 | DIC |
| ATOM | 1818 | O | ARG | 233 | 35.814 | 59.244 | 75.975 | 1.00 | 9.03 | DIC |
| ATOM | 1819 | N | THR | 234 | 34.490 | 60.132 | 77.564 | 1.00 | 8.98 | DIC |
| ATOM | 1820 | CA | THR | 234 | 33.559 | 60.693 | 76.592 | 1.00 | 10.49 | DIC |
| ATOM | 1821 | CB | THR | 234 | 32.141 | 60.811 | 77.183 | 1.00 | 10.70 | DIC |
| ATOM | 1822 | OG1 | THR | 234 | 32.181 | 61.585 | 78.387 | 1.00 | 9.78 | DIC |
| ATOM | 1823 | CG2 | THR | 234 | 31.587 | 59.416 | 77.488 | 1.00 | 8.75 | DIC |
| ATOM | 1824 | C | THR | 234 | 33.993 | 62.035 | 76.016 | 1.00 | 11.41 | DIC |
| ATOM | 1825 | O | THR | 234 | 33.481 | 62.459 | 74.982 | 1.00 | 11.24 | DIC |
| ATOM | 1826 | N | ASN | 235 | 34.924 | 62.710 | 76.681 | 1.00 | 9.83 | DIC |
| ATOM | 1827 | CA | ASN | 235 | 35.418 | 63.968 | 76.148 | 1.00 | 12.54 | DIC |
| ATOM | 1828 | CB | ASN | 235 | 36.045 | 64.845 | 77.232 | 1.00 | 15.96 | DIC |
| ATOM | 1829 | CG | ASN | 235 | 36.643 | 66.120 | 76.663 | 1.00 | 20.34 | DIC |
| ATOM | 1830 | OD1 | ASN | 235 | 35.940 | 66.934 | 76.063 | 1.00 | 25.44 | DIC |
| ATOM | 1831 | ND2 | ASN | 235 | 37.948 | 66.295 | 76.838 | 1.00 | 22.52 | DIC |
| ATOM | 1832 | C | ASN | 235 | 36.488 | 63.561 | 75.151 | 1.00 | 11.14 | DIC |
| ATOM | 1833 | O | ASN | 235 | 37.586 | 63.169 | 75.536 | 1.00 | 11.10 | DIC |
| ATOM | 1834 | N | THR | 236 | 36.154 | 63.638 | 73.870 | 1.00 | 11.67 | DIC |
| ATOM | 1835 | CA | THR | 236 | 37.081 | 63.260 | 72.813 | 1.00 | 11.17 | DIC |
| ATOM | 1836 | CB | THR | 236 | 36.413 | 63.396 | 71.433 | 1.00 | 11.34 | DIC |
| ATOM | 1837 | OG1 | THR | 236 | 35.373 | 62.417 | 71.318 | 1.00 | 10.79 | DIC |
| ATOM | 1838 | CG2 | THR | 236 | 37.431 | 63.197 | 70.315 | 1.00 | 9.33 | DIC |
| ATOM | 1839 | C | THR | 236 | 38.355 | 64.090 | 72.827 | 1.00 | 11.07 | DIC |
| ATOM | 1840 | O | THR | 236 | 38.307 | 65.320 | 72.919 | 1.00 | 11.47 | DIC |
| ATOM | 1841 | N | VAL | 237 | 39.494 | 63.409 | 72.754 | 1.00 | 10.07 | DIC |
| ATOM | 1842 | CA | VAL | 237 | 40.785 | 64.086 | 72.729 | 1.00 | 12.15 | DIC |
| ATOM | 1843 | CB | VAL | 237 | 41.865 | 63.310 | 73.523 | 1.00 | 12.90 | DIC |
| ATOM | 1844 | CG1 | VAL | 237 | 43.208 | 64.035 | 73.423 | 1.00 | 13.23 | DIC |
| ATOM | 1845 | CG2 | VAL | 237 | 41.448 | 63.183 | 74.980 | 1.00 | 13.18 | DIC |
| ATOM | 1846 | C | VAL | 237 | 41.210 | 64.177 | 71.268 | 1.00 | 12.16 | DIC |
| ATOM | 1847 | O | VAL | 237 | 41.583 | 63.175 | 70.654 | 1.00 | 12.69 | DIC |
| ATOM | 1848 | N | MSE | 238 | 41.133 | 65.381 | 70.714 | 1.00 | 12.20 | DIC |
| ATOM | 1849 | CA | MSE | 238 | 41.495 | 65.611 | 69.320 | 1.00 | 12.91 | DIC |
| ATOM | 1850 | CB | MSE | 238 | 41.283 | 67.081 | 68.960 | 1.00 | 15.07 | DIC |
| ATOM | 1851 | CG | MSE | 238 | 39.831 | 67.539 | 69.018 | 1.00 | 19.98 | DIC |
| ATOM | 1852 | SE | MSE | 238 | 38.678 | 66.551 | 67.809 | 1.00 | 27.67 | DIC |
| ATOM | 1853 | CE | MSE | 238 | 37.206 | 66.207 | 69.005 | 1.00 | 27.27 | DIC |
| ATOM | 1854 | C | MSE | 238 | 42.942 | 65.221 | 69.044 | 1.00 | 12.33 | DIC |
| ATOM | 1855 | O | MSE | 238 | 43.784 | 65.268 | 69.938 | 1.00 | 12.98 | DIC |
| ATOM | 1856 | N | PRO | 239 | 43.248 | 64.827 | 67.795 | 1.00 | 11.27 | DIC |
| ATOM | 1857 | CD | PRO | 239 | 44.633 | 64.625 | 67.335 | 1.00 | 13.15 | DIC |
| ATOM | 1858 | CA | PRO | 239 | 42.314 | 64.730 | 66.667 | 1.00 | 11.13 | DIC |
| ATOM | 1859 | CB | PRO | 239 | 43.239 | 64.787 | 65.455 | 1.00 | 12.20 | DIC |
| ATOM | 1860 | CG | PRO | 239 | 44.434 | 64.051 | 65.942 | 1.00 | 12.26 | DIC |
| ATOM | 1861 | C | PRO | 239 | 41.507 | 63.437 | 66.693 | 1.00 | 11.12 | DIC |
| ATOM | 1862 | O | PRO | 239 | 41.875 | 62.481 | 67.381 | 1.00 | 11.27 | DIC |
| ATOM | 1863 | N | VAL | 240 | 40.406 | 63.411 | 65.947 | 1.00 | 10.78 | DIC |
| ATOM | 1864 | CA | VAL | 240 | 39.582 | 62.212 | 65.873 | 1.00 | 8.88 | DIC |
| ATOM | 1865 | CB | VAL | 240 | 38.332 | 62.435 | 64.987 | 1.00 | 10.97 | DIC |
| ATOM | 1866 | CG1 | VAL | 240 | 37.571 | 61.120 | 64.817 | 1.00 | 11.55 | DIC |
| ATOM | 1867 | CG2 | VAL | 240 | 37.428 | 63.490 | 65.617 | 1.00 | 10.39 | DIC |
| ATOM | 1868 | C | VAL | 240 | 40.464 | 61.143 | 65.239 | 1.00 | 8.25 | DIC |
| ATOM | 1869 | O | VAL | 240 | 41.174 | 61.419 | 64.273 | 1.00 | 8.08 | DIC |
| ATOM | 1870 | N | ALA | 241 | 40.448 | 59.939 | 65.802 | 1.00 | 5.68 | DIC |
| ATOM | 1871 | CA | ALA | 241 | 41.255 | 58.844 | 65.277 | 1.00 | 5.69 | DIC |
| ATOM | 1872 | CB | ALA | 241 | 41.887 | 58.055 | 66.424 | 1.00 | 5.65 | DIC |
| ATOM | 1873 | C | ALA | 241 | 40.389 | 57.929 | 64.424 | 1.00 | 6.50 | DIC |
| ATOM | 1874 | O | ALA | 241 | 40.853 | 57.370 | 63.431 | 1.00 | 4.72 | DIC |
| ATOM | 1875 | N | VAL | 242 | 39.124 | 57.786 | 64.809 | 1.00 | 6.23 | DIC |
| ATOM | 1876 | CA | VAL | 242 | 38.204 | 56.940 | 64.058 | 1.00 | 5.56 | DIC |
| ATOM | 1877 | CB | VAL | 242 | 37.989 | 55.571 | 64.759 | 1.00 | 5.40 | DIC |
| ATOM | 1878 | CG1 | VAL | 242 | 37.029 | 54.707 | 63.939 | 1.00 | 5.43 | DIC |
| ATOM | 1879 | CG2 | VAL | 242 | 39.325 | 54.859 | 64.941 | 1.00 | 6.26 | DIC |
| ATOM | 1880 | C | VAL | 242 | 36.838 | 57.583 | 63.879 | 1.00 | 6.91 | DIC |
| ATOM | 1881 | O | VAL | 242 | 36.149 | 57.868 | 64.860 | 1.00 | 6.73 | DIC |
| ATOM | 1882 | N | SER | 243 | 36.462 | 57.823 | 62.623 | 1.00 | 6.89 | DIC |
| ATOM | 1883 | CA | SER | 243 | 35.148 | 58.370 | 62.300 | 1.00 | 7.37 | DIC |
| ATOM | 1884 | CB | SER | 243 | 35.177 | 59.164 | 60.985 | 1.00 | 9.26 | DIC |
| ATOM | 1885 | OG | SER | 243 | 35.627 | 60.492 | 61.187 | 1.00 | 12.73 | DIC |
| ATOM | 1886 | C | SER | 243 | 34.248 | 57.153 | 62.120 | 1.00 | 7.64 | DIC |
| ATOM | 1887 | O | SER | 243 | 34.542 | 56.279 | 61.309 | 1.00 | 7.80 | DIC |
| ATOM | 1888 | N | HIS | 244 | 33.164 | 57.082 | 62.882 | 1.00 | 6.24 | DIC |
| ATOM | 1889 | CA | HIS | 244 | 32.247 | 55.952 | 62.779 | 1.00 | 7.04 | DIC |
| ATOM | 1890 | CB | HIS | 244 | 32.209 | 55.183 | 64.103 | 1.00 | 5.69 | DIC |
| ATOM | 1891 | CG | HIS | 244 | 31.327 | 53.973 | 64.075 | 1.00 | 5.79 | DIC |
| ATOM | 1892 | CD2 | HIS | 244 | 30.668 | 53.373 | 63.055 | 1.00 | 5.25 | DIC |
| ATOM | 1893 | ND1 | HIS | 244 | 31.040 | 53.235 | 65.204 | 1.00 | 5.99 | DIC |
| ATOM | 1894 | CE1 | HIS | 244 | 30.242 | 52.233 | 64.880 | 1.00 | 8.18 | DIC |
| ATOM | 1895 | NE2 | HIS | 244 | 30.001 | 52.294 | 63.583 | 1.00 | 4.85 | DIC |
| ATOM | 1896 | C | HIS | 244 | 30.853 | 56.464 | 62.424 | 1.00 | 6.27 | DIC |
| ATOM | 1897 | O | HIS | 244 | 30.195 | 57.118 | 63.230 | 1.00 | 7.86 | DIC |
| ATOM | 1898 | N | TYR | 245 | 30.410 | 56.148 | 61.213 | 1.00 | 6.95 | DIC |
| ATOM | 1899 | CA | TYR | 245 | 29.112 | 56.585 | 60.719 | 1.00 | 8.61 | DIC |
| ATOM | 1900 | CB | TYR | 245 | 29.187 | 56.715 | 59.198 | 1.00 | 9.86 | DIC |
| ATOM | 1901 | CG | TYR | 245 | 30.263 | 57.699 | 58.815 | 1.00 | 10.60 | DIC |
| ATOM | 1902 | CD1 | TYR | 245 | 30.062 | 59.070 | 58.974 | 1.00 | 12.04 | DIC |
| ATOM | 1903 | CE1 | TYR | 245 | 31.090 | 59.983 | 58.752 | 1.00 | 12.10 | DIC |
| ATOM | 1904 | CD2 | TYR | 245 | 31.524 | 57.262 | 58.411 | 1.00 | 12.41 | DIC |
| ATOM | 1905 | CE2 | TYR | 245 | 32.564 | 58.169 | 58.187 | 1.00 | 12.40 | DIC |
| ATOM | 1906 | CZ | TYR | 245 | 32.339 | 59.527 | 58.364 | 1.00 | 12.62 | DIC |
| ATOM | 1907 | OH | TYR | 245 | 33.364 | 60.428 | 58.185 | 1.00 | 13.88 | DIC |
| ATOM | 1908 | C | TYR | 245 | 27.988 | 55.658 | 61.147 | 1.00 | 9.69 | DIC |
| ATOM | 1909 | O | TYR | 245 | 27.229 | 55.142 | 60.319 | 1.00 | 9.49 | DIC |
| ATOM | 1910 | N | CYS | 246 | 27.895 | 55.471 | 62.461 | 1.00 | 9.14 | DIC |
| ATOM | 1911 | CA | CYS | 246 | 26.888 | 54.618 | 63.071 | 1.00 | 11.69 | DIC |
| ATOM | 1912 | CB | CYS | 246 | 27.054 | 54.634 | 64.595 | 1.00 | 11.19 | DIC |
| ATOM | 1913 | SG | CYS | 246 | 26.047 | 53.422 | 65.477 | 1.00 | 17.18 | DIC |
| ATOM | 1914 | C | CYS | 246 | 25.511 | 55.147 | 62.689 | 1.00 | 12.03 | DIC |
| ATOM | 1915 | O | CYS | 246 | 25.273 | 56.351 | 62.722 | 1.00 | 11.82 | DIC |
| ATOM | 1916 | N | GLY | 247 | 24.607 | 54.246 | 62.323 | 1.00 | 12.35 | DIC |
| ATOM | 1917 | CA | GLY | 247 | 23.275 | 54.674 | 61.940 | 1.00 | 12.18 | DIC |
| ATOM | 1918 | C | GLY | 247 | 23.036 | 54.572 | 60.443 | 1.00 | 13.09 | DIC |
| ATOM | 1919 | O | GLY | 247 | 23.949 | 54.244 | 59.685 | 1.00 | 12.04 | DIC |
| ATOM | 1920 | N | PRO | 248 | 21.811 | 54.874 | 59.985 | 1.00 | 14.33 | DIC |
| ATOM | 1921 | CD | PRO | 248 | 20.738 | 55.471 | 60.803 | 1.00 | 14.23 | DIC |
| ATOM | 1922 | CA | PRO | 248 | 21.412 | 54.819 | 58.575 | 1.00 | 15.43 | DIC |
| ATOM | 1923 | CB | PRO | 248 | 19.940 | 55.227 | 58.625 | 1.00 | 17.10 | DIC |
| ATOM | 1924 | CG | PRO | 248 | 19.912 | 56.199 | 59.769 | 1.00 | 16.43 | DIC |
| ATOM | 1925 | C | PRO | 248 | 22.214 | 55.678 | 57.594 | 1.00 | 14.95 | DIC |
| ATOM | 1926 | O | PRO | 248 | 22.395 | 55.294 | 56.441 | 1.00 | 15.51 | DIC |
| ATOM | 1927 | N | ALA | 249 | 22.692 | 56.832 | 58.044 | 1.00 | 15.80 | DIC |
| ATOM | 1928 | CA | ALA | 249 | 23.442 | 57.737 | 57.175 | 1.00 | 16.01 | DIC |
| ATOM | 1929 | CB | ALA | 249 | 23.403 | 59.152 | 57.748 | 1.00 | 17.18 | DIC |
| ATOM | 1930 | C | ALA | 249 | 24.889 | 57.294 | 56.964 | 1.00 | 16.42 | DIC |
| ATOM | 1931 | O | ALA | 249 | 25.748 | 57.533 | 57.811 | 1.00 | 16.26 | DIC |
| ATOM | 1932 | N | LYS | 250 | 25.152 | 56.658 | 55.825 | 1.00 | 14.75 | DIC |
| ATOM | 1933 | CA | LYS | 250 | 26.490 | 56.169 | 55.506 | 1.00 | 14.92 | DIC |
| ATOM | 1934 | CB | LYS | 250 | 26.407 | 54.766 | 54.901 | 1.00 | 11.81 | DIC |
| ATOM | 1935 | CG | LYS | 250 | 25.716 | 53.736 | 55.783 | 1.00 | 11.87 | DIC |
| ATOM | 1936 | CD | LYS | 250 | 26.383 | 53.603 | 57.153 | 1.00 | 10.01 | DIC |
| ATOM | 1937 | CE | LYS | 250 | 25.710 | 52.507 | 57.983 | 1.00 | 10.47 | DIC |
| ATOM | 1938 | NZ | LYS | 250 | 26.130 | 52.549 | 59.415 | 1.00 | 8.71 | DIC |
| ATOM | 1939 | C | LYS | 250 | 27.225 | 57.093 | 54.542 | 1.00 | 15.74 | DIC |
| ATOM | 1940 | O | LYS | 250 | 26.609 | 57.737 | 53.695 | 1.00 | 16.86 | DIC |
| ATOM | 1941 | N | PRO | 251 | 28.563 | 57.152 | 54.646 | 1.00 | 16.85 | DIC |
| ATOM | 1942 | CD | PRO | 251 | 29.427 | 56.337 | 55.520 | 1.00 | 16.47 | DIC |
| ATOM | 1943 | CA | PRO | 251 | 29.369 | 58.010 | 53.771 | 1.00 | 17.98 | DIC |
| ATOM | 1944 | CB | PRO | 251 | 30.782 | 57.836 | 54.326 | 1.00 | 16.79 | DIC |
| ATOM | 1945 | CG | PRO | 251 | 30.769 | 56.421 | 54.821 | 1.00 | 18.00 | DIC |
| ATOM | 1946 | C | PRO | 251 | 29.270 | 57.654 | 52.289 | 1.00 | 19.21 | DIC |
| ATOM | 1947 | O | PRO | 251 | 29.493 | 58.504 | 51.425 | 1.00 | 20.56 | DIC |
| ATOM | 1948 | N | TRP | 252 | 28.936 | 56.401 | 51.993 | 1.00 | 20.23 | DIC |
| ATOM | 1949 | CA | TRP | 252 | 28.807 | 55.966 | 50.604 | 1.00 | 20.95 | DIC |
| ATOM | 1950 | CB | TRP | 252 | 29.134 | 54.476 | 50.483 | 1.00 | 18.75 | DIC |
| ATOM | 1951 | CG | TRP | 252 | 28.377 | 53.604 | 51.431 | 1.00 | 15.29 | DIC |
| ATOM | 1952 | CD2 | TRP | 252 | 28.895 | 52.971 | 52.607 | 1.00 | 13.13 | DIC |
| ATOM | 1953 | CE2 | TRP | 252 | 27.839 | 52.236 | 53.185 | 1.00 | 11.47 | DIC |
| ATOM | 1954 | CE3 | TRP | 252 | 30.151 | 52.952 | 53.227 | 1.00 | 11.22 | DIC |
| ATOM | 1955 | CD1 | TRP | 252 | 27.065 | 53.243 | 51.347 | 1.00 | 14.01 | DIC |
| ATOM | 1956 | NE1 | TRP | 252 | 26.733 | 52.420 | 52.397 | 1.00 | 12.89 | DIC |
| ATOM | 1957 | CZ2 | TRP | 252 | 27.998 | 51.488 | 54.357 | 1.00 | 11.76 | DIC |
| ATOM | 1958 | CZ3 | TRP | 252 | 30.311 | 52.207 | 54.395 | 1.00 | 10.17 | DIC |
| ATOM | 1959 | CH2 | TRP | 252 | 29.239 | 51.486 | 54.945 | 1.00 | 9.82 | DIC |
| ATOM | 1960 | C | TRP | 252 | 27.415 | 56.249 | 50.046 | 1.00 | 23.17 | DIC |
| ATOM | 1961 | O | TRP | 252 | 27.069 | 55.808 | 48.948 | 1.00 | 24.13 | DIC |
| ATOM | 1962 | N | HIS | 253 | 26.620 | 56.990 | 50.809 | 1.00 | 24.69 | DIC |
| ATOM | 1963 | CA | HIS | 253 | 25.273 | 57.351 | 50.388 | 1.00 | 27.61 | DIC |
| ATOM | 1964 | CB | HIS | 253 | 24.364 | 57.523 | 51.605 | 1.00 | 26.78 | DIC |
| ATOM | 1965 | CG | HIS | 253 | 23.843 | 56.233 | 52.156 | 1.00 | 27.30 | DIC |
| ATOM | 1966 | CD2 | HIS | 253 | 23.890 | 54.972 | 51.666 | 1.00 | 26.61 | DIC |
| ATOM | 1967 | ND1 | HIS | 253 | 23.147 | 56.159 | 53.343 | 1.00 | 27.44 | DIC |
| ATOM | 1968 | CE1 | HIS | 253 | 22.786 | 54.907 | 53.561 | 1.00 | 27.37 | DIC |
| ATOM | 1969 | NE2 | HIS | 253 | 23.224 | 54.167 | 52.559 | 1.00 | 27.99 | DIC |
| ATOM | 1970 | C | HIS | 253 | 25.293 | 58.645 | 49.582 | 1.00 | 29.67 | DIC |
| ATOM | 1971 | O | HIS | 253 | 24.324 | 58.968 | 48.892 | 1.00 | 14.02 | DIC |
| ATOM | 1972 | N | ALA | 258 | 27.264 | 63.867 | 56.914 | 1.00 | 29.28 | DIC |
| ATOM | 1973 | CA | ALA | 258 | 27.941 | 63.637 | 58.185 | 1.00 | 27.86 | DIC |
| ATOM | 1974 | CB | ALA | 258 | 27.889 | 62.162 | 58.545 | 1.00 | 26.95 | DIC |
| ATOM | 1975 | C | ALA | 258 | 29.388 | 64.107 | 58.096 | 1.00 | 26.15 | DIC |
| ATOM | 1976 | O | ALA | 258 | 29.995 | 64.081 | 57.026 | 1.00 | 26.60 | DIC |
| ATOM | 1977 | N | TRP | 259 | 29.933 | 64.540 | 59.228 | 1.00 | 25.33 | DIC |
| ATOM | 1978 | CA | TRP | 259 | 31.305 | 65.026 | 59.282 | 1.00 | 24.17 | DIC |
| ATOM | 1979 | CB | TRP | 259 | 31.678 | 65.406 | 60.716 | 1.00 | 24.67 | DIC |
| ATOM | 1980 | CG | TRP | 259 | 33.088 | 65.907 | 60.841 | 1.00 | 24.17 | DIC |
| ATOM | 1981 | CD2 | TRP | 259 | 34.222 | 65.166 | 61.307 | 1.00 | 23.57 | DIC |
| ATOM | 1982 | CE2 | TRP | 259 | 35.347 | 66.012 | 61.199 | 1.00 | 24.08 | DIC |
| ATOM | 1983 | CE3 | TRP | 259 | 34.398 | 63.867 | 61.803 | 1.00 | 23.76 | DIC |
| ATOM | 1984 | CD1 | TRP | 259 | 33.556 | 67.137 | 60.482 | 1.00 | 24.26 | DIC |
| ATOM | 1985 | NE1 | TRP | 259 | 34.913 | 67.209 | 60.693 | 1.00 | 24.21 | DIC |
| ATOM | 1986 | CZ2 | TRP | 259 | 36.633 | 65.602 | 61.571 | 1.00 | 24.47 | DIC |
| ATOM | 1987 | CZ3 | TRP | 259 | 35.678 | 63.459 | 62.172 | 1.00 | 21.73 | DIC |
| ATOM | 1988 | CH2 | TRP | 259 | 36.776 | 64.324 | 62.054 | 1.00 | 23.77 | DIC |
| ATOM | 1989 | C | TRP | 259 | 32.319 | 64.006 | 58.770 | 1.00 | 24.50 | DIC |
| ATOM | 1990 | O | TRP | 259 | 32.183 | 62.804 | 59.006 | 1.00 | 24.08 | DIC |
| ATOM | 1991 | N | GLY | 260 | 33.333 | 64.503 | 58.065 | 1.00 | 22.84 | DIC |
| ATOM | 1992 | CA | GLY | 260 | 34.389 | 63.651 | 57.545 | 1.00 | 22.82 | DIC |
| ATOM | 1993 | C | GLY | 260 | 34.026 | 62.699 | 56.421 | 1.00 | 22.52 | DIC |
| ATOM | 1994 | O | GLY | 260 | 34.883 | 61.959 | 55.944 | 1.00 | 22.25 | DIC |
| ATOM | 1995 | N | ALA | 261 | 32.769 | 62.713 | 55.992 | 1.00 | 23.04 | DIC |
| ATOM | 1996 | CA | ALA | 261 | 32.324 | 61.830 | 54.923 | 1.00 | 22.66 | DIC |
| ATOM | 1997 | CB | ALA | 261 | 30.849 | 62.081 | 54.623 | 1.00 | 24.10 | DIC |
| ATOM | 1998 | C | ALA | 261 | 33.156 | 61.989 | 53.648 | 1.00 | 23.16 | DIC |
| ATOM | 1999 | O | ALA | 261 | 33.369 | 61.023 | 52.915 | 1.00 | 24.06 | DIC |
| ATOM | 2000 | N | ALA | 262 | 33.628 | 63.205 | 53.388 | 1.00 | 22.44 | DIC |
| ATOM | 2001 | CA | ALA | 262 | 34.423 | 63.489 | 52.194 | 1.00 | 21.08 | DIC |
| ATOM | 2002 | CB | ALA | 262 | 34.829 | 64.960 | 52.174 | 1.00 | 22.66 | DIC |
| ATOM | 2003 | C | ALA | 262 | 35.663 | 62.616 | 52.058 | 1.00 | 20.72 | DIC |
| ATOM | 2004 | O | ALA | 262 | 36.129 | 62.368 | 50.943 | 1.00 | 18.77 | DIC |
| ATOM | 2005 | N | ARG | 263 | 36.208 | 62.163 | 53.184 | 1.00 | 18.78 | DIC |
| ATOM | 2006 | CA | ARG | 263 | 37.397 | 61.318 | 53.153 | 1.00 | 18.76 | DIC |
| ATOM | 2007 | CB | ARG | 263 | 37.924 | 61.067 | 54.570 | 1.00 | 21.15 | DIC |
| ATOM | 2008 | CG | ARG | 263 | 39.094 | 60.079 | 54.635 | 1.00 | 25.36 | DIC |
| ATOM | 2009 | CD | ARG | 263 | 39.670 | 59.996 | 56.051 | 1.00 | 30.63 | DIC |
| ATOM | 2010 | NE | ARG | 263 | 40.709 | 58.973 | 56.199 | 1.00 | 32.82 | DIC |
| ATOM | 2011 | CZ | ARG | 263 | 41.814 | 58.901 | 55.460 | 1.00 | 34.60 | DIC |
| ATOM | 2012 | NH1 | ARG | 263 | 42.696 | 57.933 | 55.681 | 1.00 | 34.80 | DIC |
| ATOM | 2013 | NH2 | ARG | 263 | 42.037 | 59.785 | 54.495 | 1.00 | 34.28 | DIC |
| ATOM | 2014 | C | ARG | 263 | 37.089 | 59.991 | 52.478 | 1.00 | 17.13 | DIC |
| ATOM | 2015 | O | ARG | 263 | 37.963 | 59.384 | 51.862 | 1.00 | 16.27 | DIC |
| ATOM | 2016 | N | PHE | 264 | 35.844 | 59.540 | 52.599 | 1.00 | 15.52 | DIC |
| ATOM | 2017 | CA | PHE | 264 | 35.437 | 58.284 | 51.983 | 1.00 | 15.68 | DIC |
| ATOM | 2018 | CB | PHE | 264 | 33.970 | 57.978 | 52.293 | 1.00 | 15.52 | DIC |
| ATOM | 2019 | CG | PHE | 264 | 33.495 | 56.678 | 51.716 | 1.00 | 14.73 | DIC |
| ATOM | 2020 | CD1 | PHE | 264 | 33.570 | 55.504 | 52.459 | 1.00 | 15.37 | DIC |
| ATOM | 2021 | CD2 | PHE | 264 | 33.009 | 56.617 | 50.412 | 1.00 | 14.26 | DIC |
| ATOM | 2022 | CE1 | PHE | 264 | 33.170 | 54.289 | 51.915 | 1.00 | 14.89 | DIC |
| ATOM | 2023 | CE2 | PHE | 264 | 32.606 | 55.405 | 49.856 | 1.00 | 13.60 | DIC |
| ATOM | 2024 | CZ | PHE | 264 | 32.687 | 54.239 | 50.609 | 1.00 | 14.32 | DIC |
| ATOM | 2025 | C | PHE | 264 | 35.611 | 58.372 | 50.469 | 1.00 | 16.23 | DIC |
| ATOM | 2026 | O | PHE | 264 | 36.271 | 57.529 | 49.858 | 1.00 | 16.76 | DIC |
| ATOM | 2027 | N | THR | 265 | 35.014 | 59.398 | 49.870 | 1.00 | 17.19 | DIC |
| ATOM | 2028 | CA | THR | 265 | 35.092 | 59.587 | 48.426 | 1.00 | 19.52 | DIC |
| ATOM | 2029 | CB | THR | 265 | 34.227 | 60.789 | 47.970 | 1.00 | 21.53 | DIC |
| ATOM | 2030 | OG1 | THR | 265 | 34.141 | 60.802 | 46.538 | 1.00 | 24.56 | DIC |
| ATOM | 2031 | CG2 | THR | 265 | 34.832 | 62.095 | 48.437 | 1.00 | 22.63 | DIC |
| ATOM | 2032 | C | THR | 265 | 36.539 | 59.788 | 47.971 | 1.00 | 19.59 | DIC |
| ATOM | 2033 | O | THR | 265 | 36.938 | 59.308 | 46.910 | 1.00 | 18.82 | DIC |
| ATOM | 2034 | N | GLU | 266 | 37.331 | 60.485 | 48.780 | 1.00 | 18.91 | DIC |
| ATOM | 2035 | CA | GLU | 266 | 38.729 | 60.710 | 48.435 | 1.00 | 19.77 | DIC |
| ATOM | 2036 | CB | GLU | 266 | 39.394 | 61.635 | 49.450 | 1.00 | 22.93 | DIC |
| ATOM | 2037 | CG | GLU | 266 | 40.890 | 61.785 | 49.227 | 1.00 | 29.46 | DIC |
| ATOM | 2038 | CD | GLU | 266 | 41.516 | 62.808 | 50.146 | 1.00 | 32.08 | DIC |
| ATOM | 2039 | OE1 | GLU | 266 | 41.433 | 62.632 | 51.380 | 1.00 | 34.07 | DIC |
| ATOM | 2040 | OE2 | GLU | 266 | 42.091 | 63.790 | 49.631 | 1.00 | 35.23 | DIC |
| ATOM | 2041 | C | GLU | 266 | 39.488 | 59.386 | 48.384 | 1.00 | 18.19 | DIC |
| ATOM | 2042 | O | GLU | 266 | 40.345 | 59.185 | 47.525 | 1.00 | 16.67 | DIC |
| ATOM | 2043 | N | LEU | 267 | 39.177 | 58.488 | 49.315 | 1.00 | 16.93 | DIC |
| ATOM | 2044 | CA | LEU | 267 | 39.823 | 57.181 | 49.362 | 1.00 | 15.79 | DIC |
| ATOM | 2045 | CB | LEU | 267 | 39.571 | 56.509 | 50.715 | 1.00 | 17.37 | DIC |
| ATOM | 2046 | CG | LEU | 267 | 40.497 | 56.943 | 51.851 | 1.00 | 16.52 | DIC |
| ATOM | 2047 | CD1 | LEU | 267 | 40.007 | 56.374 | 53.165 | 1.00 | 17.75 | DIC |
| ATOM | 2048 | CD2 | LEU | 267 | 41.911 | 56.466 | 51.549 | 1.00 | 16.20 | DIC |
| ATOM | 2049 | C | LEU | 267 | 39.320 | 56.279 | 48.243 | 1.00 | 15.71 | DIC |
| ATOM | 2050 | O | LEU | 267 | 40.096 | 55.550 | 47.623 | 1.00 | 14.15 | DIC |
| ATOM | 2051 | N | ALA | 268 | 38.018 | 56.330 | 47.984 | 1.00 | 16.10 | DIC |
| ATOM | 2052 | CA | ALA | 268 | 37.430 | 55.511 | 46.932 | 1.00 | 18.09 | DIC |
| ATOM | 2053 | CB | ALA | 268 | 35.919 | 55.683 | 46.915 | 1.00 | 18.61 | DIC |
| ATOM | 2054 | C | ALA | 268 | 38.019 | 55.901 | 45.581 | 1.00 | 19.91 | DIC |
| ATOM | 2055 | O | ALA | 268 | 38.128 | 55.072 | 44.680 | 1.00 | 20.47 | DIC |
| ATOM | 2056 | N | GLY | 269 | 38.404 | 57.165 | 45.447 | 1.00 | 20.77 | DIC |
| ATOM | 2057 | CA | GLY | 269 | 38.974 | 57.627 | 44.196 | 1.00 | 22.67 | DIC |
| ATOM | 2058 | C | GLY | 269 | 40.431 | 57.241 | 44.014 | 1.00 | 23.70 | DIC |
| ATOM | 2059 | O | GLY | 269 | 40.946 | 57.266 | 42.898 | 1.00 | 24.52 | DIC |
| ATOM | 2060 | N | SER | 270 | 41.096 | 56.879 | 45.107 | 1.00 | 24.12 | DIC |
| ATOM | 2061 | CA | SER | 270 | 42.505 | 56.498 | 45.054 | 1.00 | 24.97 | DIC |
| ATOM | 2062 | CB | SER | 270 | 43.210 | 56.903 | 46.350 | 1.00 | 25.49 | DIC |
| ATOM | 2063 | OG | SER | 270 | 42.733 | 56.143 | 47.448 | 1.00 | 27.01 | DIC |
| ATOM | 2064 | C | SER | 270 | 42.687 | 55.001 | 44.842 | 1.00 | 25.34 | DIC |
| ATOM | 2065 | O | SER | 270 | 43.813 | 54.511 | 44.769 | 1.00 | 25.69 | DIC |
| ATOM | 2066 | N | LEU | 271 | 41.578 | 54.276 | 44.742 | 1.00 | 25.34 | DIC |
| ATOM | 2067 | CA | LEU | 271 | 41.633 | 52.834 | 44.558 | 1.00 | 25.82 | DIC |
| ATOM | 2068 | CB | LEU | 271 | 40.218 | 52.247 | 44.564 | 1.00 | 24.31 | DIC |
| ATOM | 2069 | CG | LEU | 271 | 39.497 | 52.396 | 45.907 | 1.00 | 23.12 | DIC |
| ATOM | 2070 | CD1 | LEU | 271 | 38.112 | 51.772 | 45.833 | 1.00 | 23.63 | DIC |
| ATOM | 2071 | CD2 | LEU | 271 | 40.328 | 51.732 | 46.999 | 1.00 | 21.99 | DIC |
| ATOM | 2072 | C | LEU | 271 | 42.370 | 52.410 | 43.295 | 1.00 | 26.79 | DIC |
| ATOM | 2073 | O | LEU | 271 | 42.110 | 52.916 | 42.205 | 1.00 | 26.57 | DIC |
| ATOM | 2074 | N | THR | 272 | 43.297 | 51.474 | 43.469 | 1.00 | 27.68 | DIC |
| ATOM | 2075 | CA | THR | 272 | 44.099 | 50.940 | 42.378 | 1.00 | 28.62 | DIC |
| ATOM | 2076 | CB | THR | 272 | 45.090 | 49.884 | 42.902 | 1.00 | 28.86 | DIC |
| ATOM | 2077 | OG1 | THR | 272 | 45.988 | 50.496 | 43.835 | 1.00 | 31.54 | DIC |
| ATOM | 2078 | CG2 | THR | 272 | 45.882 | 49.278 | 41.757 | 1.00 | 29.83 | DIC |
| ATOM | 2079 | C | THR | 272 | 43.219 | 50.290 | 41.320 | 1.00 | 28.48 | DIC |
| ATOM | 2080 | O | THR | 272 | 43.315 | 50.615 | 40.136 | 1.00 | 27.52 | DIC |
| ATOM | 2081 | N | THR | 273 | 42.368 | 49.366 | 41.759 | 1.00 | 28.31 | DIC |
| ATOM | 2082 | CA | THR | 273 | 41.463 | 48.649 | 40.867 | 1.00 | 27.75 | DIC |
| ATOM | 2083 | CB | THR | 273 | 41.776 | 47.136 | 40.851 | 1.00 | 29.35 | DIC |
| ATOM | 2084 | OG1 | THR | 273 | 43.155 | 46.931 | 40.515 | 1.00 | 31.31 | DIC |
| ATOM | 2085 | CG2 | THR | 273 | 40.900 | 46.426 | 39.828 | 1.00 | 31.25 | DIC |
| ATOM | 2086 | C | THR | 273 | 40.018 | 48.824 | 41.324 | 1.00 | 25.84 | DIC |
| ATOM | 2087 | O | THR | 273 | 39.695 | 48.590 | 42.489 | 1.00 | 24.34 | DIC |
| ATOM | 2088 | N | VAL | 274 | 39.151 | 49.230 | 40.402 | 1.00 | 22.69 | DIC |
| ATOM | 2089 | CA | VAL | 274 | 37.745 | 49.425 | 40.725 | 1.00 | 20.88 | DIC |
| ATOM | 2090 | CB | VAL | 274 | 37.378 | 50.932 | 40.744 | 1.00 | 19.63 | DIC |
| ATOM | 2091 | CG1 | VAL | 274 | 37.700 | 51.569 | 39.405 | 1.00 | 21.82 | DIC |
| ATOM | 2092 | CG2 | VAL | 274 | 35.906 | 51.106 | 41.077 | 1.00 | 20.86 | DIC |
| ATOM | 2093 | C | VAL | 274 | 36.851 | 48.684 | 39.735 | 1.00 | 19.45 | DIC |
| ATOM | 2094 | O | VAL | 274 | 36.644 | 49.129 | 38.604 | 1.00 | 19.49 | DIC |
| ATOM | 2095 | N | PRO | 275 | 36.317 | 47.524 | 40.151 | 1.00 | 18.18 | DIC |
| ATOM | 2096 | CD | PRO | 275 | 36.580 | 46.851 | 41.433 | 1.00 | 16.49 | DIC |
| ATOM | 2097 | CA | PRO | 275 | 35.441 | 46.705 | 39.311 | 1.00 | 17.39 | DIC |
| ATOM | 2098 | CB | PRO | 275 | 35.079 | 45.540 | 40.230 | 1.00 | 17.76 | DIC |
| ATOM | 2099 | CG | PRO | 275 | 36.283 | 45.416 | 41.097 | 1.00 | 17.23 | DIC |
| ATOM | 2100 | C | PRO | 275 | 34.208 | 47.476 | 38.858 | 1.00 | 16.95 | DIC |
| ATOM | 2101 | O | PRO | 275 | 33.642 | 48.257 | 39.619 | 1.00 | 14.46 | DIC |
| ATOM | 2102 | N | GLU | 276 | 33.799 | 47.257 | 37.613 | 1.00 | 18.62 | DIC |
| ATOM | 2103 | CA | GLU | 276 | 32.620 | 47.924 | 37.070 | 1.00 | 19.68 | DIC |
| ATOM | 2104 | CB | GLU | 276 | 32.250 | 47.292 | 35.720 | 1.00 | 20.05 | DIC |
| ATOM | 2105 | CG | GLU | 276 | 30.884 | 47.680 | 35.146 | 1.00 | 23.31 | DIC |
| ATOM | 2106 | CD | GLU | 276 | 30.747 | 49.162 | 34.846 | 1.00 | 24.44 | DIC |
| ATOM | 2107 | OE1 | GLU | 276 | 31.776 | 49.825 | 34.606 | 1.00 | 25.94 | DIC |
| ATOM | 2108 | OE2 | GLU | 276 | 29.601 | 49.662 | 34.834 | 1.00 | 26.03 | DIC |
| ATOM | 2109 | C | GLU | 276 | 31.453 | 47.812 | 38.056 | 1.00 | 19.58 | DIC |
| ATOM | 2110 | O | GLU | 276 | 30.705 | 48.765 | 38.255 | 1.00 | 19.51 | DIC |
| ATOM | 2111 | N | GLU | 277 | 31.325 | 46.651 | 38.693 | 1.00 | 19.98 | DIC |
| ATOM | 2112 | CA | GLU | 277 | 30.246 | 46.413 | 39.647 | 1.00 | 21.58 | DIC |
| ATOM | 2113 | CB | GLU | 277 | 30.155 | 44.916 | 39.962 | 1.00 | 25.97 | DIC |
| ATOM | 2114 | CG | GLU | 277 | 28.727 | 44.372 | 40.027 | 1.00 | 31.33 | DIC |
| ATOM | 2115 | CD | GLU | 277 | 28.073 | 44.244 | 38.653 | 1.00 | 33.58 | DIC |
| ATOM | 2116 | OE1 | GLU | 277 | 27.923 | 45.267 | 37.948 | 1.00 | 34.50 | DIC |
| ATOM | 2117 | OE2 | GLU | 277 | 27.706 | 43.111 | 38.279 | 1.00 | 36.07 | DIC |
| ATOM | 2118 | C | GLU | 277 | 30.383 | 47.210 | 40.953 | 1.00 | 21.12 | DIC |
| ATOM | 2119 | O | GLU | 277 | 29.507 | 47.148 | 41.816 | 1.00 | 21.31 | DIC |
| ATOM | 2120 | N | TRP | 278 | 31.481 | 47.949 | 41.097 | 1.00 | 19.40 | DIC |
| ATOM | 2121 | CA | TRP | 278 | 31.719 | 48.772 | 42.286 | 1.00 | 18.57 | DIC |
| ATOM | 2122 | CB | TRP | 279 | 33.174 | 48.652 | 42.744 | 1.00 | 16.02 | DIC |
| ATOM | 2123 | CG | TRP | 278 | 33.510 | 47.418 | 43.518 | 1.00 | 13.84 | DIC |
| ATOM | 2124 | CD2 | TRP | 278 | 34.666 | 47.224 | 44.339 | 1.00 | 13.65 | DIC |
| ATOM | 2125 | CE2 | TRP | 278 | 34.617 | 45.899 | 44.823 | 1.00 | 12.87 | DIC |
| ATOM | 2126 | CE3 | TRP | 278 | 35.742 | 48.042 | 44.711 | 1.00 | 11.77 | DIC |
| ATOM | 2127 | CD1 | TRP | 278 | 32.822 | 46.240 | 43.539 | 1.00 | 13.38 | DIC |
| ATOM | 2128 | NE1 | TRP | 278 | 33.481 | 45.321 | 44.321 | 1.00 | 14.26 | DIC |
| ATOM | 2129 | CZ2 | TRP | 278 | 35.604 | 45.371 | 45.661 | 1.00 | 11.66 | DIC |
| ATOM | 2130 | CZ3 | TRP | 278 | 36.724 | 47.516 | 45.546 | 1.00 | 12.57 | DIC |
| ATOM | 2131 | CH2 | TRP | 278 | 36.646 | 46.192 | 46.010 | 1.00 | 11.95 | DIC |
| ATOM | 2132 | C | TRP | 278 | 31.443 | 50.240 | 41.977 | 1.00 | 19.09 | DIC |
| ATOM | 2133 | O | TRP | 278 | 31.266 | 51.051 | 42.885 | 1.00 | 17.92 | DIC |
| ATOM | 2134 | N | ALA | 279 | 31.422 | 50.572 | 40.690 | 1.00 | 21.02 | DIC. |
| ATOM | 2135 | CA | ALA | 279 | 31.200 | 51.943 | 40.235 | 1.00 | 23.01 | DIC |
| ATOM | 2136 | CB | ALA | 279 | 31.061 | 51.969 | 38.717 | 1.00 | 24.70 | DIC |
| ATOM | 2137 | C | ALA | 279 | 29.997 | 52.619 | 40.875 | 1.00 | 23.44 | DIC |
| ATOM | 2138 | O | ALA | 279 | 30.088 | 53.764 | 41.317 | 1.00 | 25.20 | DIC |
| ATOM | 2139 | N | ALA | 280 | 28.871 | 51.917 | 40.918 | 1.00 | 24.09 | DIC |
| ATOM | 2140 | CA | ALA | 280 | 27.656 | 52.470 | 41.504 | 1.00 | 24.89 | DIC |
| ATOM | 2141 | CB | ALA | 280 | 26.460 | 51.598 | 41.140 | 1.00 | 25.25 | DIC |
| ATOM | 2142 | C | ALA | 280 | 27.765 | 52.604 | 43.024 | 1.00 | 25.44 | DIC |
| ATOM | 2143 | O | ALA | 280 | 27.449 | 53.655 | 43.586 | 1.00 | 26.12 | DIC |
| ATOM | 2144 | N | ALA | 281 | 28.214 | 51.538 | 43.680 | 1.00 | 24.53 | DIC |
| ATOM | 2145 | CA | ALA | 281 | 28.363 | 51.529 | 45.132 | 1.00 | 24.14 | DIC |
| ATOM | 2146 | CB | ALA | 281 | 28.895 | 50.176 | 45.592 | 1.00 | 22.30 | DIC |
| ATOM | 2147 | C | ALA | 281 | 29.301 | 52.637 | 45.590 | 1.00 | 24.69 | DIC |
| ATOM | 2148 | O | ALA | 281 | 29.097 | 53.231 | 46.649 | 1.00 | 24.21 | DIC |
| ATOM | 2149 | N | ALA | 282 | 30.326 | 52.890 | 44.775 | 1.00 | 25.23 | DIC |
| ATOM | 2150 | CA | ALA | 282 | 31.354 | 53.907 | 45.008 | 1.00 | 28.24 | DIC |
| ATOM | 2151 | CB | ALA | 282 | 30.889 | 54.936 | 46.040 | 1.00 | 29.57 | DIC |
| ATOM | 2152 | C | ALA | 282 | 32.672 | 53.285 | 45.456 | 1.00 | 29.28 | DIC |
| ATOM | 2153 | OT1 | ALA | 282 | 32.749 | 52.040 | 45.554 | 1.00 | 30.11 | DIC |
| ATOM | 2154 | OT2 | ALA | 282 | 33.619 | 54.061 | 45.703 | 1.00 | 31.66 | DIC |
| ATOM | 2155 | N1 | UPG | 341 | 27.143 | 44.459 | 57.507 | 1.00 | 10.01 | |
| ATOM | 2156 | C2 | UPG | 341 | 27.525 | 43.800 | 56.322 | 1.00 | 9.52 | |
| ATOM | 2157 | N3 | UPG | 341 | 26.652 | 44.070 | 55.245 | 1.00 | 8.67 | |
| ATOM | 2158 | C4 | UPG | 341 | 25.480 | 44.911 | 55.273 | 1.00 | 8.79 | |
| ATOM | 2159 | C5 | UPG | 341 | 25.173 | 45.541 | 56.522 | 1.00 | 8.07 | |
| ATOM | 2160 | C6 | UPG | 341 | 25.965 | 45.316 | 57.564 | 1.00 | 8.80 | |
| ATOM | 2161 | O2 | UPG | 341 | 28.508 | 43.077 | 56.249 | 1.00 | 9.10 | |
| ATOM | 2162 | O4 | UPG | 341 | 24.821 | 45.047 | 54.262 | 1.00 | 10.92 | |
| ATOM | 2163 | C4* | UPG | 341 | 28.064 | 45.814 | 60.802 | 1.00 | 9.49 | |
| ATOM | 2164 | O4* | UPG | 341 | 27.241 | 44.769 | 60.117 | 1.00 | 9.77 | |
| ATOM | 2165 | C3* | UPG | 341 | 28.852 | 46.439 | 59.647 | 1.00 | 8.11 | |
| ATOM | 2166 | O3* | UPG | 341 | 29.963 | 47.181 | 60.117 | 1.00 | 10.37 | |
| ATOM | 2167 | C2* | UPG | 341 | 29.109 | 45.217 | 58.781 | 1.00 | 9.07 | |
| ATOM | 2168 | O2* | UPG | 341 | 30.227 | 44.448 | 59.212 | 1.00 | 9.05 | |
| ATOM | 2169 | C1* | UPG | 341 | 27.829 | 44.408 | 58.886 | 1.00 | 10.20 | |
| ATOM | 2170 | C5* | UPG | 341 | 26.745 | 46.233 | 61.382 | 1.00 | 8.16 | |
| ATOM | 2171 | O5* | UPG | 341 | 26.201 | 47.589 | 61.086 | 1.00 | 14.00 | |
| ATOM | 2172 | PA | UPG | 341 | 26.684 | 49.097 | 60.762 | 1.00 | 11.18 | |
| ATOM | 2173 | O1A | UPG | 341 | 28.145 | 49.287 | 60.708 | 1.00 | 12.79 | |
| ATOM | 2174 | O2A | UPG | 341 | 25.988 | 49.605 | 59.534 | 1.00 | 13.97 | |
| ATOM | 2175 | O3A | UPG | 341 | 25.956 | 49.273 | 62.186 | 1.00 | 12.14 | |
| ATOM | 2176 | PB | UPG | 341 | 25.671 | 50.458 | 63.209 | 1.00 | 14.34 | |
| ATOM | 2177 | O1B | UPG | 341 | 26.996 | 51.096 | 63.328 | 1.00 | 11.37 | |
| ATOM | 2178 | O2B | UPG | 341 | 24.689 | 51.364 | 62.606 | 1.00 | 15.46 | |
| ATOM | 2179 | O3B | UPG | 341 | 25.195 | 49.937 | 64.603 | 1.00 | 15.30 | |
| ATOM | 2180 | C1′ | UPG | 341 | 25.560 | 49.791 | 66.150 | 1.00 | 20.50 | |
| ATOM | 2181 | C2′ | UPG | 341 | 27.035 | 49.261 | 66.121 | 1.00 | 19.24 | |
| ATOM | 2182 | C3′ | UPG | 341 | 27.359 | 48.017 | 65.147 | 1.00 | 20.09 | |
| ATOM | 2183 | C4′ | UPG | 341 | 26.330 | 46.877 | 65.447 | 1.00 | 19.60 | |
| ATOM | 2184 | C5′ | UPG | 341 | 24.895 | 47.560 | 65.400 | 1.00 | 19.91 | |
| ATOM | 2185 | C6′ | UPG | 341 | 23.873 | 46.533 | 65.880 | 1.00 | 21.17 | |
| ATOM | 2186 | F2′ | UPG | 341 | 27.959 | 50.293 | 65.853 | 1.00 | 22.12 | |
| ATOM | 2187 | O3′ | UPG | 341 | 28.644 | 47.531 | 65.431 | 1.00 | 18.85 | |
| ATOM | 2188 | O4′ | UPG | 341 | 26.604 | 46.316 | 66.765 | 1.00 | 17.85 | |
| ATOM | 2189 | O5′ | UPG | 341 | 24.675 | 48.684 | 66.396 | 1.00 | 19.90 | |
| ATOM | 2190 | O6′ | UPG | 341 | 23.634 | 45.281 | 65.305 | 1.00 | 20.17 | |
| ATOM | 2191 | MN | MN | 400 | 28.972 | 50.701 | 62.315 | 1.00 | 5.95 | MN |
| ATOM | 2192 | O | HOH | 500 | 33.706 | 51.230 | 70.721 | 1.00 | 5.79 | |
| ATOM | 2193 | O | HOH | 501 | 34.343 | 56.571 | 75.304 | 1.00 | 6.75 | |
| ATOM | 2194 | O | HOH | 502 | 17.802 | 43.967 | 68.994 | 1.00 | 10.59 | |
| ATOM | 2195 | O | HOH | 503 | 30.228 | 40.611 | 62.764 | 1.00 | 5.01 | |
| ATOM | 2196 | O | HOH | 504 | 39.307 | 27.582 | 66.202 | 1.00 | 10.52 | |
| ATOM | 2197 | O | HOH | 505 | 28.063 | 58.310 | 70.871 | 1.00 | 11.73 | |
| ATOM | 2198 | O | HOH | 506 | 28.710 | 46.590 | 79.022 | 1.00 | 6.65 | |
| ATOM | 2199 | O | HOH | 507 | 52.557 | 49.070 | 68.969 | 1.00 | 12.93 | |
| ATOM | 2200 | O | HOH | 508 | 45.198 | 58.379 | 72.300 | 1.00 | 14.17 | |
| ATOM | 2201 | O | HOH | 509 | 34.051 | 65.873 | 73.044 | 1.00 | 11.35 | |
| ATOM | 2202 | O | HOH | 510 | 41.782 | 55.536 | 74.876 | 1.00 | 9.63 | |
| ATOM | 2203 | O | HOH | 511 | 34.074 | 33.978 | 70.193 | 1.00 | 11.60 | |
| ATOM | 2204 | O | HOH | 512 | 26.575 | 37.907 | 50.900 | 1.00 | 12.73 | |
| ATOM | 2205 | O | HOH | 513 | 20.508 | 53.715 | 78.759 | 1.00 | 13.23 | |
| ATOM | 2206 | O | HOH | 514 | 51.349 | 46.691 | 65.666 | 1.00 | 17.16 | |
| ATOM | 2207 | O | HOH | 515 | 46.546 | 58.564 | 67.349 | 1.00 | 8.65 | |
| ATOM | 2208 | O | HOH | 516 | 52.691 | 53.505 | 61.177 | 1.00 | 12.18 | |
| ATOM | 2209 | O | HOH | 517 | 32.821 | 62.545 | 72.466 | 1.00 | 11.60 | |
| ATOM | 2210 | O | HOH | 518 | 25.540 | 58.412 | 67.321 | 1.00 | 14.19 | |
| ATOM | 2211 | O | HOH | 519 | 43.067 | 31.129 | 75.720 | 1.00 | 15.28 | |
| ATOM | 2212 | O | HOH | 520 | 42.729 | 59.876 | 74.613 | 1.00 | 15.64 | |
| ATOM | 2213 | O | HOH | 521 | 22.179 | 51.497 | 64.101 | 1.00 | 18.51 | |
| ATOM | 2214 | O | HOH | 522 | 34.014 | 47.551 | 83.195 | 1.00 | 9.04 | |
| ATOM | 2215 | O | HOH | 523 | 31.143 | 47.689 | 82.928 | 1.00 | 12.50 | |
| ATOM | 2216 | O | HOH | 524 | 32.337 | 57.530 | 71.624 | 1.00 | 8.71 | |
| ATOM | 2217 | O | HOH | 525 | 32.452 | 44.011 | 37.817 | 1.00 | 14.80 | |
| ATOM | 2218 | O | HOH | 526 | 24.863 | 45.001 | 86.150 | 1.00 | 15.13 | |
| ATOM | 2219 | O | HOH | 527 | 30.407 | 45.125 | 46.996 | 1.00 | 21.84 | |
| ATOM | 2220 | O | HOH | 528 | 20.754 | 39.640 | 56.706 | 1.00 | 9.45 | |
| ATOM | 2221 | O | HOH | 529 | 38.854 | 52.122 | 81.581 | 1.00 | 10.97 | |
| ATOM | 2222 | O | HOH | 530 | 40.378 | 59.034 | 77.824 | 1.00 | 17.34 | |
| ATOM | 2223 | O | HOH | 531 | 45.109 | 40.792 | 78.558 | 1.00 | 18.88 | |
| ATOM | 2224 | O | HOH | 532 | 48.994 | 58.795 | 68.032 | 1.00 | 18.00 | |
| ATOM | 2225 | O | HOH | 533 | 40.483 | 67.706 | 72.634 | 1.00 | 23.92 | |
| ATOM | 2226 | O | HOH | 534 | 41.280 | 60.433 | 45.444 | 1.00 | 22.17 | |
| ATOM | 2227 | O | HOH | 535 | 26.497 | 64.111 | 54.278 | 1.00 | 24.53 | |
| ATOM | 2228 | O | HOH | 536 | 45.960 | 43.860 | 76.459 | 1.00 | 17.37 | |
| ATOM | 2229 | O | HOH | 537 | 14.734 | 48.868 | 65.365 | 1.00 | 18.59 | |
| ATOM | 2230 | O | HOH | 538 | 46.131 | 38.545 | 57.874 | 1.00 | 24.34 | |
| ATOM | 2231 | O | HOH | 539 | 28.264 | 60.048 | 87.951 | 1.00 | 24.52 | |
| ATOM | 2232 | O | HOH | 540 | 46.748 | 45.401 | 73.816 | 1.00 | 16.95 | |
| ATOM | 2233 | O | HOH | 541 | 37.416 | 49.298 | 83.116 | 1.00 | 21.65 | |
| ATOM | 2234 | O | HOH | 542 | 27.237 | 60.848 | 55.024 | 1.00 | 18.79 | |
| ATOM | 2235 | O | HOH | 543 | 19.040 | 32.149 | 71.513 | 1.00 | 19.39 | |
| ATOM | 2236 | O | HOH | 544 | 24.859 | 59.199 | 87.172 | 1.00 | 21.96 | |
| ATOM | 2237 | O | HOH | 545 | 31.727 | 30.205 | 70.890 | 1.00 | 15.96 | |
| ATOM | 2238 | O | HOH | 546 | 50.448 | 55.331 | 62.596 | 1.00 | 19.00 | |
| ATOM | 2239 | O | HOH | 547 | 44.684 | 61.061 | 73.050 | 1.00 | 18.55 | |
| ATOM | 2240 | O | HOH | 548 | 23.917 | 28.286 | 65.355 | 1.00 | 22.75 | |
| ATOM | 2241 | O | HOH | 549 | 48.030 | 45.080 | 55.012 | 1.00 | 18.04 | |
| ATOM | 2242 | O | HOH | 550 | 31.863 | 50.861 | 84.807 | 1.00 | 12.35 | |
| ATOM | 2243 | O | HOH | 551 | 47.230 | 50.648 | 79.238 | 1.00 | 20.70 | |
| ATOM | 2244 | O | HOH | 552 | 45.574 | 59.143 | 57.227 | 1.00 | 26.33 | |
| ATOM | 2245 | O | HOH | 553 | 30.141 | 30.242 | 60.911 | 1.00 | 18.49 | |
| ATOM | 2246 | O | HOH | 554 | 39.361 | 63.494 | 77.807 | 1.00 | 19.60 | |
| ATOM | 2247 | O | HOH | 555 | 47.878 | 40.139 | 68.058 | 1.00 | 19.90 | |
| ATOM | 2248 | O | HOH | 556 | 33.763 | 51.759 | 54.205 | 1.00 | 21.40 | |
| ATOM | 2249 | O | HOH | 557 | 26.346 | 51.553 | 89.495 | 1.00 | 25.04 | |
| ATOM | 2250 | O | HOH | 558 | 43.622 | 40.021 | 44.147 | 1.00 | 18.14 | |
| ATOM | 2251 | O | HOH | 559 | 22.590 | 60.674 | 51.245 | 1.00 | 20.28 | |
| ATOM | 2252 | O | HOH | 560 | 46.899 | 45.705 | 42.406 | 1.00 | 23.24 | |
| ATOM | 2253 | O | HOH | 561 | 22.785 | 41.493 | 81.141 | 1.00 | 16.19 | |
| ATOM | 2254 | O | HOH | 562 | 24.738 | 38.970 | 55.796 | 1.00 | 22.33 | |
| ATOM | 2255 | O | HOH | 563 | 44.428 | 51.002 | 46.157 | 1.00 | 14.80 | |
| ATOM | 2256 | O | HOH | 564 | 24.412 | 40.573 | 48.290 | 1.00 | 23.85 | |
| ATOM | 2257 | O | HOH | 565 | 28.192 | 56.913 | 87.234 | 1.00 | 17.82 | |
| ATOM | 2258 | O | HOH | 566 | 49.326 | 45.339 | 71.775 | 1.00 | 22.08 | |
| ATOM | 2259 | O | HOH | 567 | 13.438 | 46.500 | 69.972 | 1.00 | 21.87 | |
| ATOM | 2260 | O | HOH | 568 | 36.472 | 30.850 | 71.825 | 1.00 | 24.81 | |
| ATOM | 2261 | O | HOH | 569 | 48.009 | 36.876 | 62.329 | 1.00 | 23.86 | |
| ATOM | 2262 | O | HOH | 570 | 24.860 | 62.779 | 55.872 | 1.00 | 26.97 | |
| ATOM | 2263 | O | HOH | 571 | 33.245 | 67.145 | 56.945 | 1.00 | 20.42 | |
| ATOM | 2264 | O | HOH | 572 | 42.157 | 34.486 | 52.245 | 1.00 | 16.87 | |
| ATOM | 2265 | O | HOH | 573 | 50.717 | 47.209 | 75.899 | 1.00 | 22.49 | |
| ATOM | 2266 | O | HOH | 574 | 29.865 | 33.602 | 47.244 | 1.00 | 32.56 | |
| ATOM | 2267 | O | HOH | 575 | 22.722 | 37.430 | 81.986 | 1.00 | 23.93 | |
| ATOM | 2268 | O | HOH | 576 | 50.100 | 42.319 | 65.178 | 1.00 | 22.32 | |
| ATOM | 2269 | O | HOH | 577 | 57.571 | 51.761 | 56.046 | 1.00 | 21.27 | |
| ATOM | 2270 | O | HOH | 578 | 47.296 | 51.529 | 51.324 | 1.00 | 20.20 | |
| ATOM | 2271 | O | HOH | 579 | 29.733 | 32.008 | 53.762 | 1.00 | 26.39 | |
| ATOM | 2272 | O | HOH | 580 | 26.968 | 35.843 | 81.316 | 1.00 | 22.87 | |
| ATOM | 2273 | O | HOH | 581 | 14.080 | 40.066 | 63.324 | 1.00 | 21.92 | |
| ATOM | 2274 | O | HOH | 582 | 28.224 | 49.947 | 38.771 | 1.00 | 25.67 | |
| ATOM | 2275 | O | HOH | 583 | 31.203 | 53.364 | 87.667 | 1.00 | 15.87 | |
| ATOM | 2276 | O | HOH | 584 | 42.912 | 37.536 | 80.236 | 1.00 | 23.43 | |
| ATOM | 2277 | O | HOH | 585 | 22.186 | 39.308 | 53.880 | 1.00 | 20.38 | |
| ATOM | 2278 | O | HOH | 586 | 48.511 | 56.150 | 75.023 | 1.00 | 32.95 | |
| ATOM | 2279 | O | HOH | 587 | 24.730 | 34.535 | 78.912 | 1.00 | 18.02 | |
| ATOM | 2280 | O | HOH | 588 | 32.604 | 39.346 | 83.395 | 1.00 | 22.21 | |
| ATOM | 2281 | O | HOH | 589 | 41.240 | 63.564 | 62.618 | 1.00 | 10.36 | |
| ATOM | 2282 | O | HOH | 590 | 26.537 | 56.699 | 69.106 | 1.00 | 8.22 | |
| ATOM | 2283 | O | HOH | 591 | 52.797 | 47.471 | 74.334 | 1.00 | 9.21 | |
| ATOM | 2284 | O | HOH | 592 | 41.777 | 32.558 | 61.182 | 1.00 | 10.73 | |
| ATOM | 2285 | O | HOH | 593 | 33.437 | 44.877 | 60.209 | 1.00 | 7.34 | |
| ATOM | 2286 | O | HOH | 594 | 33.888 | 31.650 | 71.550 | 1.00 | 14.92 | |
| ATOM | 2287 | O | HOH | 595 | 25.462 | 40.367 | 50.723 | 1.00 | 11.84 | |
| ATOM | 2288 | O | HOH | 596 | 43.591 | 57.443 | 74.474 | 1.00 | 17.30 | |
| ATOM | 2289 | O | HOH | 597 | 33.115 | 35.658 | 41.851 | 1.00 | 14.12 | |
| ATOM | 2290 | O | HOH | 598 | 44.433 | 41.685 | 76.122 | 1.00 | 16.31 | |
| ATOM | 2291 | O | HOH | 599 | 29.834 | 27.545 | 68.444 | 1.00 | 22.39 | |
| ATOM | 2292 | O | HOH | 600 | 37.953 | 40.975 | 40.829 | 1.00 | 14.27 | |
| ATOM | 2293 | O | HOH | 601 | 12.155 | 45.294 | 67.909 | 1.00 | 17.65 | |
| ATOM | 2294 | O | HOH | 602 | 22.482 | 36.971 | 50.284 | 1.00 | 24.49 | |
| ATOM | 2295 | O | HOH | 603 | 23.544 | 68.425 | 75.283 | 1.00 | 28.58 | |
| ATOM | 2296 | O | HOH | 604 | 45.872 | 62.745 | 70.934 | 1.00 | 20.85 | |
| ATOM | 2297 | O | HOH | 605 | 27.938 | 49.214 | 42.120 | 1.00 | 30.40 | |
| ATOM | 2298 | O | HOH | 606 | 27.096 | 61.728 | 78.244 | 1.00 | 24.23 | |
| ATOM | 2299 | O | HOH | 607 | 31.086 | 37.382 | 40.546 | 1.00 | 19.00 | |
| ATOM | 2300 | O | HOH | 608 | 41.624 | 62.545 | 78.909 | 1.00 | 18.97 | |
| ATOM | 2301 | O | HOH | 609 | 41.687 | 56.553 | 77.779 | 1.00 | 20.00 | |
| ATOM | 2302 | O | HOH | 610 | 24.401 | 57.605 | 65.060 | 1.00 | 18.11 | |
| ATOM | 2303 | O | HOH | 611 | 22.321 | 34.679 | 80.031 | 1.00 | 20.17 | |
| ATOM | 2304 | O | HOH | 612 | 50.721 | 43.533 | 70.330 | 1.00 | 19.94 | |
| ATOM | 2305 | O | HOH | 613 | 13.420 | 52.527 | 51.343 | 1.00 | 25.95 | |
| ATOM | 2306 | O | HOH | 614 | 24.279 | 27.169 | 60.916 | 1.00 | 21.76 | |
| ATOM | 2307 | O | HOH | 615 | 34.037 | 52.439 | 85.454 | 1.00 | 17.26 | |
| ATOM | 2308 | O | HOH | 616 | 39.054 | 65.244 | 80.148 | 1.00 | 24.89 | |
| ATOM | 2309 | O | HOH | 617 | 39.054 | 64.302 | 51.723 | 1.00 | 29.39 | |
| ATOM | 2310 | O | HOH | 618 | 43.601 | 62.259 | 58.842 | 1.00 | 27.04 | |
| ATOM | 2311 | O | HOH | 619 | 22.409 | 56.766 | 90.280 | 1.00 | 33.32 | |
| ATOM | 2312 | O | HOH | 620 | 20.846 | 28.993 | 74.299 | 1.00 | 20.46 | |
| ATOM | 2313 | O | HOH | 621 | 17.253 | 46.648 | 86.012 | 1.00 | 32.49 | |
| ATOM | 2314 | O | HOH | 622 | 21.298 | 51.633 | 88.129 | 1.00 | 31.62 | |
| ATOM | 2315 | O | HOH | 623 | 28.632 | 26.574 | 61.870 | 1.00 | 27.33 | |
| ATOM | 2316 | O | HOH | 624 | 48.345 | 58.417 | 61.097 | 1.00 | 25.77 | |
| ATOM | 2317 | O | HOH | 625 | 36.305 | 67.237 | 72.635 | 1.00 | 20.04 | |
| ATOM | 2318 | O | HOH | 626 | 42.598 | 53.012 | 75.632 | 1.00 | 19.40 | |
| ATOM | 2319 | O | HOH | 627 | 20.218 | 47.782 | 86.726 | 1.00 | 22.94 | |
| ATOM | 2320 | O | HOH | 628 | 32.159 | 63.722 | 48.806 | 1.00 | 23.31 | |
| ATOM | 2321 | O | HOH | 629 | 23.413 | 63.225 | 51.020 | 1.00 | 26.53 | |
| ATOM | 2322 | O | HOH | 630 | 29.942 | 43.073 | 43.112 | 1.00 | 28.69 | |
| ATOM | 2323 | O | HOH | 631 | 26.522 | 26.069 | 67.811 | 1.00 | 29.08 | |
| ATOM | 2324 | O | HOH | 632 | 22.688 | 56.444 | 82.013 | 1.00 | 24.60 | |
| ATOM | 2325 | O | HOH | 633 | 52.189 | 43.104 | 63.856 | 1.00 | 22.68 | |
| ATOM | 2326 | O | HOH | 634 | 25.706 | 64.912 | 64.257 | 1.00 | 22.84 | |
| ATOM | 2327 | O | HOH | 635 | 36.511 | 40.834 | 83.556 | 1.00 | 23.63 | |
| ATOM | 2328 | O | HOH | 636 | 31.226 | 27.212 | 64.879 | 1.00 | 21.79 | |
| ATOM | 2329 | O | HOH | 637 | 40.945 | 34.644 | 81.702 | 1.00 | 23.90 | |
| ATOM | 2330 | O | HOH | 638 | 21.448 | 59.284 | 81.673 | 1.00 | 34.56 | |
| ATOM | 2331 | O | HOH | 639 | 49.130 | 60.035 | 70.370 | 1.00 | 30.49 | |
| ATOM | 2332 | O | HOH | 640 | 45.879 | 54.032 | 77.176 | 1.00 | 28.11 | |
| ATOM | 2333 | O | HOH | 641 | 41.217 | 55.181 | 84.434 | 1.00 | 27.45 | |
| ATOM | 2334 | O | HOH | 642 | 15.917 | 59.351 | 77.084 | 1.00 | 22.78 | |
| ATOM | 2335 | O | HOH | 643 | 26.422 | 58.999 | 59.868 | 1.00 | 26.25 | |
| ATOM | 2336 | O | HOH | 644 | 23.973 | 36.865 | 54.522 | 1.00 | 23.25 | |
| ATOM | 2337 | O | HOH | 645 | 23.524 | 48.998 | 51.566 | 1.00 | 30.36 | |
| ATOM | 2338 | O | HOH | 646 | 46.194 | 61.930 | 74.996 | 1.00 | 35.99 | |
| ATOM | 2339 | O | HOH | 647 | 9.547 | 44.920 | 75.322 | 1.00 | 30.40 | |
| ATOM | 2340 | O | HOH | 648 | 40.501 | 29.426 | 67.866 | 1.00 | 27.54 | |
| ATOM | 2341 | O | HOH | 649 | 42.953 | 47.204 | 81.751 | 1.00 | 28.53 | |
| ATOM | 2342 | O | HOH | 650 | 26.628 | 46.271 | 47.232 | 1.00 | 28.74 | |
| ATOM | 2343 | O | HOH | 651 | 41.609 | 36.542 | 46.766 | 1.00 | 26.30 | |
| ATOM | 2344 | O | HOH | 652 | 47.789 | 62.434 | 66.963 | 1.00 | 22.24 | |
| ATOM | 2345 | O | HOH | 653 | 49.409 | 58.883 | 57.890 | 1.00 | 32.71 | |
| ATOM | 2346 | O | HOH | 654 | 17.321 | 49.855 | 67.521 | 1.00 | 17.23 | |
| ATOM | 2347 | O | HOH | 655 | 51.495 | 46.932 | 72.085 | 1.00 | 20.28 | |
| ATOM | 2348 | O | HOH | 656 | 34.466 | 27.259 | 73.207 | 1.00 | 21.37 | |
| ATOM | 2349 | O | HOH | 657 | 38.002 | 69.000 | 71.442 | 1.00 | 14.96 | |
| ATOM | 2350 | O | HOH | 658 | 20.746 | 31.132 | 65.541 | 1.00 | 23.69 | |
| ATOM | 2351 | O | HOH | 659 | 58.157 | 43.951 | 58.062 | 1.00 | 20.32 | |
| ATOM | 2352 | O | HOH | 660 | 38.437 | 33.849 | 81.870 | 1.00 | 20.83 | |
| ATOM | 2353 | O | HOH | 661 | 43.258 | 34.683 | 60.615 | 1.00 | 17.70 | |
| ATOM | 2354 | O | HOH | 662 | 36.377 | 51.103 | 84.600 | 1.00 | 27.30 | |
| ATOM | 2355 | O | HOH | 663 | 25.933 | 38.583 | 81.765 | 1.00 | 24.70 | |
| ATOM | 2356 | O | HOH | 664 | 29.974 | 47.696 | 49.284 | 1.00 | 24.16 | |
| ATOM | 2357 | O | HOH | 665 | 49.678 | 57.581 | 72.793 | 1.00 | 26.38 | |
| ATOM | 2358 | O | HOH | 666 | 50.099 | 56.413 | 54.533 | 1.00 | 21.01 | |
| ATOM | 2359 | O | HOH | 667 | 52.662 | 42.022 | 61.588 | 1.00 | 28.15 | |
| ATOM | 2360 | O | HOH | 668 | 20.130 | 54.995 | 81.261 | 1.00 | 24.64 | |
| ATOM | 2361 | O | HOH | 669 | 46.100 | 40.982 | 44.473 | 1.00 | 25.43 | |
| ATOM | 2362 | O | HOH | 670 | 47.498 | 47.532 | 45.696 | 1.00 | 27.31 | |
| ATOM | 2363 | O | HOH | 671 | 32.535 | 33.305 | 43.257 | 1.00 | 27.22 | |
| ATOM | 2364 | O | HOH | 672 | 43.715 | 31.852 | 63.188 | 1.00 | 19.52 | |
| ATOM | 2365 | O | HOH | 673 | 24.492 | 65.745 | 55.161 | 1.00 | 28.73 | |
| ATOM | 2366 | O | HOH | 674 | 27.731 | 64.219 | 61.902 | 1.00 | 29.97 | |
| ATOM | 2367 | O | HOH | 675 | 44.865 | 49.755 | 80.491 | 1.00 | 26.39 | |
| ATOM | 2368 | O | HOH | 676 | 37.157 | 68.382 | 83.515 | 1.00 | 28.68 | |
| ATOM | 2369 | O | HOH | 677 | 30.154 | 55.040 | 89.681 | 1.00 | 29.35 | |
| ATOM | 2370 | O | HOH | 678 | 30.684 | 39.377 | 42.310 | 1.00 | 17.22 | |
| ATOM | 2371 | O | HOH | 679 | 18.954 | 28.710 | 77.300 | 1.00 | 34.29 | |
| ATOM | 2372 | O | HOH | 680 | 36.128 | 60.169 | 43.709 | 1.00 | 32.96 | |
| ATOM | 2373 | O | HOH | 681 | 41.469 | 52.484 | 78.099 | 1.00 | 23.04 | |
| ATOM | 2374 | O | HOH | 682 | 26.597 | 36.390 | 48.638 | 1.00 | 26.34 | |
| ATOM | 2375 | O | HOH | 683 | 50.624 | 46.042 | 56.343 | 1.00 | 27.15 | |
| ATOM | 2376 | O | HOH | 684 | 32.352 | 60.908 | 62.085 | 1.00 | 29.37 | |
| ATOM | 2377 | O | HOH | 685 | 36.543 | 42.286 | 39.079 | 1.00 | 36.28 | |
| ATOM | 2378 | O | HOH | 686 | 13.775 | 50.845 | 82.171 | 1.00 | 28.05 | |
| ATOM | 2379 | O | HOH | 687 | 47.886 | 43.395 | 52.513 | 1.00 | 28.22 | |
| ATOM | 2380 | O | HOH | 688 | 50.808 | 53.317 | 53.903 | 1.00 | 33.82 | |
| ATOM | 2381 | O | HOH | 689 | 33.470 | 54.665 | 86.847 | 1.00 | 17.58 | |
| ATOM | 2382 | O | HOH | 690 | 19.330 | 58.463 | 73.532 | 1.00 | 17.09 | |
| ATOM | 2383 | O | HOH | 691 | 51.990 | 49.111 | 66.439 | 1.00 | 25.06 | |
| ATOM | 2384 | O | HOH | 692 | 55.576 | 56.592 | 60.334 | 1.00 | 24.70 | |
| ATOM | 2385 | O | HOH | 693 | 50.867 | 41.843 | 47.444 | 1.00 | 32.36 | |
| ATOM | 2386 | O | HOH | 694 | 28.359 | 36.216 | 43.792 | 1.00 | 27.71 | |
| ATOM | 2387 | O | HOH | 695 | 30.229 | 40.375 | 82.671 | 1.00 | 15.17 | |
| ATOM | 2388 | O | HOH | 696 | 34.080 | 42.336 | 41.876 | 1.00 | 15.17 | |
| ATOM | 2389 | C | ACY | 244 | 23.704 | 53.254 | 68.309 | 1.00 | 20.17 | |
| ATOM | 2390 | O | ACY | 244 | 24.283 | 54.249 | 68.921 | 1.00 | 20.17 | |
| ATOM | 2391 | OXT | ACY | 244 | 23.988 | 52.013 | 68.413 | 1.00 | 20.17 | |
| ATOM | 2392 | CH3 | ACY | 244 | 22.577 | 53.681 | 67.378 | 1.00 | 20.17 | |

TABLE 5
REMARK coordinates from minimization refinement
REMARK refinement resolution: 20.0-2.0 A
REMARK starting r = .2565 free_r = .2833
REMARK final r = .2545 free_r = .2823
REMARK rmsd bonds = .006693 rmsd angles = 1.31286
REMARK wa = 1.49748
REMARK target = mlf cycles = 1 steps = 25
REMARK sg = P2 (1) 2 (1) 2 (1) a = 39.975 b = 76.158 c = 87.228
alpha = 90 beta = 90 gamma = 90
REMARK parameter file 1: CNS_TOPPAR: protein_rep.param
REMARK parameter file 2: ../upg.par
REMARK parameter file 3: CNS_TOPPAR: ion.param
REMARK parameter file 4: CNS_TOPPAR: water_rep.param
REMARK parameter file 5: ../lat.par
REMARK molecular structure file: generate3.mtf
REMARK input coordinates: generate3.pdb
REMARK reflection file = ../../deoxyse.cv
REMARK ncs = none
REMARK B-correction resolution: 6.0-2.0
REMARK initial B-factor correction applied to fobs:
REMARK B11 = 1.331 B22 = 2.292 B33 = −3.623
REMARK B12 = .000 B13 = .000 B23 = .000
REMARK B-factor correction applied to coordinate array B: −.107
REMARK bulk solvent: density level = .364834 e/A\hat{ }3, B-factor = 28.3776 A\hat{ }2
REMARK reflections with |Fobs|/sigma_F < 0.0 rejected
REMARK reflections with |Fobs| > 10000 * rms (Fobs) rejected

| | |
|---|---|
| REMARK theoretical total number of refl. in resol. range: | 18599 (>100.0%) |
| REMARK number of unobserved reflections (no entry or \|F\| = 0): | 374 (>2.0%) |
| REMARK number of reflections rejected: | 0 (.0%) |
| REMARK total number of reflections used: | 18225 (>98.0%) |
| REMARK number of reflections in working set: | 17339 (93.2%) |
| REMARK number of reflections in test set: | 886 (>4.8%) |

CRYST1 39.975 76.158 87.228 90.00 90.00 90.00 P 21 21 21
REMARK FILENAME = “minimize3.pdb”
REMARK DATE: 7-Jun-00 00:41:57 created by user: karina
REMARK VERSION: 1.0

| | | | | | | | | | | |
|---|---|---|---|---|---|---|---|---|---|---|
| ATOM | 1 | CB | MSE | 1 | 48.137 | 46.257 | 65.175 | 1.00 | 15.20 | DIC |
| ATOM | 2 | CG | MSE | 1 | 47.648 | 47.190 | 64.082 | 1.00 | 16.41 | DIC |
| ATOM | 3 | SE | MSE | 1 | 46.777 | 48.813 | 64.794 | 1.00 | 17.84 | DIC |
| ATOM | 4 | CE | MSE | 1 | 48.349 | 49.605 | 65.593 | 1.00 | 17.32 | DIC |
| ATOM | 5 | C | MSE | 1 | 47.761 | 44.130 | 63.891 | 1.00 | 12.29 | DIC |
| ATOM | 6 | O | MSE | 1 | 47.811 | 44.149 | 62.665 | 1.00 | 12.03 | DIC |
| ATOM | 7 | N | MSE | 1 | 50.002 | 45.161 | 63.937 | 1.00 | 12.69 | DIC |
| ATOM | 8 | CA | MSE | 1 | 48.762 | 44.924 | 64.719 | 1.00 | 12.79 | DIC |
| ATOM | 9 | N | ASP | 2 | 46.865 | 43.424 | 64.570 | 1.00 | 11.18 | DIC |
| ATOM | 10 | CA | ASP | 2 | 45.852 | 42.621 | 63.902 | 1.00 | 11.07 | DIC |
| ATOM | 11 | CB | ASP | 2 | 45.779 | 41.223 | 64.516 | 1.00 | 11.12 | DIC |
| ATOM | 12 | CG | ASP | 2 | 47.054 | 40.435 | 64.305 | 1.00 | 12.17 | DIC |
| ATOM | 13 | OD1 | ASP | 2 | 47.948 | 40.480 | 65.180 | 1.00 | 11.27 | DIC |
| ATOM | 14 | OD2 | ASP | 2 | 47.164 | 39.783 | 63.247 | 1.00 | 12.68 | DIC |
| ATOM | 15 | C | ASP | 2 | 44.494 | 43.289 | 63.985 | 1.00 | 10.71 | DIC |
| ATOM | 16 | O | ASP | 2 | 43.971 | 43.534 | 65.072 | 1.00 | 10.39 | DIC |
| ATOM | 17 | N | ILE | 3 | 43.946 | 43.590 | 62.813 | 1.00 | 9.90 | DIC |
| ATOM | 18 | CA | ILE | 3 | 42.650 | 44.245 | 62.675 | 1.00 | 9.22 | DIC |
| ATOM | 19 | CB | ILE | 3 | 42.716 | 45.381 | 61.627 | 1.00 | 9.23 | DIC |
| ATOM | 20 | CG2 | ILE | 3 | 41.353 | 46.043 | 61.480 | 1.00 | 6.69 | DIC |
| ATOM | 21 | CG1 | ILE | 3 | 43.799 | 46.392 | 62.019 | 1.00 | 8.74 | DIC |
| ATOM | 22 | CD | ILE | 3 | 43.544 | 47.108 | 63.340 | 1.00 | 10.46 | DIC |
| ATOM | 23 | C | ILE | 3 | 41.649 | 43.216 | 62.186 | 1.00 | 9.22 | DIC |
| ATOM | 24 | O | ILE | 3 | 41.974 | 42.392 | 61.330 | 1.00 | 8.87 | DIC |
| ATOM | 25 | N | VAL | 4 | 40.443 | 43.262 | 62.738 | 1.00 | 9.68 | DIC |
| ATOM | 26 | CA | VAL | 4 | 39.383 | 42.346 | 62.343 | 1.00 | 9.18 | DIC |
| ATOM | 27 | CB | VAL | 4 | 38.861 | 41.512 | 63.519 | 1.00 | 9.44 | DIC |
| ATOM | 28 | CG1 | VAL | 4 | 37.758 | 40.566 | 63.024 | 1.00 | 9.88 | DIC |
| ATOM | 29 | CG2 | VAL | 4 | 39.994 | 40.741 | 64.157 | 1.00 | 9.29 | DIC |
| ATOM | 30 | C | VAL | 4 | 38.198 | 43.127 | 61.807 | 1.00 | 9.80 | DIC |
| ATOM | 31 | O | VAL | 4 | 37.805 | 44.137 | 62.381 | 1.00 | 8.97 | DIC |
| ATOM | 32 | N | PHE | 5 | 37.653 | 42.651 | 60.693 | 1.00 | 9.50 | DIC |
| ATOM | 33 | CA | PHE | 5 | 36.477 | 43.241 | 60.060 | 1.00 | 9.57 | DIC |
| ATOM | 34 | CB | PHE | 5 | 36.823 | 43.854 | 58.694 | 1.00 | 9.78 | DIC |
| ATOM | 35 | CG | PHE | 5 | 37.479 | 45.205 | 58.763 | 1.00 | 8.54 | DIC |
| ATOM | 36 | CD1 | PHE | 5 | 36.784 | 46.308 | 59.248 | 1.00 | 7.97 | DIC |
| ATOM | 37 | CD2 | PHE | 5 | 38.772 | 45.385 | 58.283 | 1.00 | 9.19 | DIC |
| ATOM | 38 | CE1 | PHE | 5 | 37.368 | 47.577 | 59.246 | 1.00 | 8.53 | DIC |
| ATOM | 39 | CE2 | PHE | 5 | 39.366 | 46.645 | 58.275 | 1.00 | 9.07 | DIC |
| ATOM | 40 | CZ | PHE | 5 | 38.661 | 47.744 | 58.757 | 1.00 | 9.04 | DIC |
| ATOM | 41 | C | PHE | 5 | 35.544 | 42.052 | 59.818 | 1.00 | 9.75 | DIC |
| ATOM | 42 | O | PHE | 5 | 35.985 | 40.896 | 59.803 | 1.00 | 8.48 | DIC |
| ATOM | 43 | N | ALA | 6 | 34.265 | 42.338 | 59.638 | 1.00 | 8.31 | DIC |
| ATOM | 44 | CA | ALA | 6 | 33.273 | 41.316 | 59.345 | 1.00 | 9.26 | DIC |
| ATOM | 45 | CB | ALA | 6 | 32.416 | 41.019 | 60.572 | 1.00 | 9.18 | DIC |
| ATOM | 46 | C | ALA | 6 | 32.424 | 41.941 | 58.252 | 1.00 | 9.59 | DIC |
| ATOM | 47 | O | ALA | 6 | 31.983 | 43.077 | 58.388 | 1.00 | 9.30 | DIC |
| ATOM | 48 | N | ALA | 7 | 32.195 | 41.218 | 57.165 | 1.00 | 9.68 | DIC |
| ATOM | 49 | CA | ALA | 7 | 31.385 | 41.777 | 56.088 | 1.00 | 9.55 | DIC |
| ATOM | 50 | CB | ALA | 7 | 32.242 | 42.714 | 55.237 | 1.00 | 10.43 | DIC |
| ATOM | 51 | C | ALA | 7 | 30.784 | 40.703 | 55.205 | 1.00 | 10.26 | DIC |
| ATOM | 52 | O | ALA | 7 | 31.358 | 39.628 | 55.069 | 1.00 | 9.81 | DIC |
| ATOM | 53 | N | ASP | 8 | 29.614 | 40.982 | 54.630 | 1.00 | 10.79 | DIC |
| ATOM | 54 | CA | ASP | 8 | 29.008 | 40.048 | 53.693 | 1.00 | 10.98 | DIC |
| ATOM | 55 | CB | ASP | 8 | 27.481 | 39.986 | 53.822 | 1.00 | 11.13 | DIC |
| ATOM | 56 | CG | ASP | 8 | 26.852 | 41.337 | 54.092 | 1.00 | 10.25 | DIC |
| ATOM | 57 | OD1 | ASP | 8 | 27.419 | 42.365 | 53.676 | 1.00 | 8.93 | DIC |
| ATOM | 58 | OD2 | ASP | 8 | 25.768 | 41.361 | 54.719 | 1.00 | 11.96 | DIC |
| ATOM | 59 | C | ASP | 8 | 29.399 | 40.620 | 52.340 | 1.00 | 12.11 | DIC |
| ATOM | 60 | O | ASP | 8 | 30.143 | 41.596 | 52.281 | 1.00 | 11.30 | DIC |
| ATOM | 61 | N | ASP | 9 | 28.903 | 40.042 | 51.255 | 1.00 | 12.32 | DIC |
| ATOM | 62 | CA | ASP | 9 | 29.269 | 40.534 | 49.931 | 1.00 | 13.23 | DIC |
| ATOM | 63 | CB | ASP | 9 | 28.701 | 39.607 | 48.852 | 1.00 | 14.44 | DIC |
| ATOM | 64 | CG | ASP | 9 | 29.286 | 39.890 | 47.486 | 1.00 | 14.65 | DIC |
| ATOM | 65 | OD1 | ASP | 9 | 30.516 | 39.787 | 47.335 | 1.00 | 13.72 | DIC |
| ATOM | 66 | OD2 | ASP | 9 | 28.520 | 40.222 | 46.567 | 1.00 | 17.00 | DIC |
| ATOM | 67 | C | ASP | 9 | 28.817 | 41.977 | 49.674 | 1.00 | 13.55 | DIC |
| ATOM | 68 | O | ASP | 9 | 29.500 | 42.737 | 48.984 | 1.00 | 12.70 | DIC |
| ATOM | 69 | N | ASN | 10 | 27.671 | 42.349 | 50.237 | 1.00 | 13.42 | DIC |
| ATOM | 70 | CA | ASN | 10 | 27.122 | 43.689 | 50.068 | 1.00 | 13.87 | DIC |
| ATOM | 71 | CB | ASN | 10 | 25.804 | 43.814 | 50.836 | 1.00 | 14.04 | DIC |
| ATOM | 72 | CG | ASN | 10 | 25.148 | 45.171 | 50.650 | 1.00 | 15.16 | DIC |
| ATOM | 73 | OD1 | ASN | 10 | 24.977 | 45.634 | 49.523 | 1.00 | 14.38 | DIC |
| ATOM | 74 | ND2 | ASN | 10 | 24.767 | 45.810 | 51.754 | 1.00 | 13.92 | DIC |
| ATOM | 75 | C | ASN | 10 | 28.074 | 44.794 | 50.527 | 1.00 | 13.56 | DIC |
| ATOM | 76 | O | ASN | 10 | 28.076 | 45.900 | 49.965 | 1.00 | 13.16 | DIC |
| ATOM | 77 | N | TYR | 11 | 28.879 | 44.492 | 51.541 | 1.00 | 12.92 | DIC |
| ATOM | 78 | CA | TYR | 11 | 29.821 | 45.460 | 52.091 | 1.00 | 13.06 | DIC |
| ATOM | 79 | CB | TYR | 11 | 29.733 | 45.440 | 53.616 | 1.00 | 13.83 | DIC |
| ATOM | 80 | CG | TYR | 11 | 28.756 | 46.430 | 54.192 | 1.00 | 14.83 | DIC |
| ATOM | 81 | CD1 | TYR | 11 | 27.652 | 46.865 | 53.459 | 1.00 | 14.49 | DIC |
| ATOM | 82 | CE1 | TYR | 11 | 26.758 | 47.782 | 53.995 | 1.00 | 15.54 | DIC |
| ATOM | 83 | CD2 | TYR | 11 | 28.934 | 46.935 | 55.480 | 1.00 | 15.47 | DIC |
| ATOM | 84 | CE2 | TYR | 11 | 28.044 | 47.849 | 56.021 | 1.00 | 16.13 | DIC |
| ATOM | 85 | CZ | TYR | 11 | 26.962 | 48.268 | 55.277 | 1.00 | 15.85 | DIC |
| ATOM | 86 | OH | TYR | 11 | 26.082 | 49.171 | 55.822 | 1.00 | 17.10 | DIC |
| ATOM | 87 | C | TYR | 11 | 31.278 | 45.291 | 51.677 | 1.00 | 13.35 | DIC |
| ATOM | 88 | O | TYR | 11 | 32.156 | 45.945 | 52.246 | 1.00 | 12.67 | DIC |
| ATOM | 89 | N | ALA | 12 | 31.540 | 44.436 | 50.689 | 1.00 | 12.77 | DIC |
| ATOM | 90 | CA | ALA | 12 | 32.905 | 44.196 | 50.226 | 1.00 | 11.93 | DIC |
| ATOM | 91 | CB | ALA | 12 | 32.898 | 43.176 | 49.085 | 1.00 | 12.43 | DIC |
| ATOM | 92 | C | ALA | 12 | 33.646 | 45.465 | 49.786 | 1.00 | 12.10 | DIC |
| ATOM | 93 | O | ALA | 12 | 34.812 | 45.659 | 50.131 | 1.00 | 9.34 | DIC |
| ATOM | 94 | N | ALA | 13 | 32.981 | 46.321 | 49.014 | 1.00 | 11.40 | DIC |
| ATOM | 95 | CA | ALA | 13 | 33.614 | 47.552 | 48.548 | 1.00 | 11.49 | DIC |
| ATOM | 96 | CB | ALA | 13 | 32.711 | 48.259 | 47.525 | 1.00 | 12.59 | DIC |
| ATOM | 97 | C | ALA | 13 | 33.935 | 48.486 | 49.719 | 1.00 | 11.12 | DIC |
| ATOM | 98 | O | ALA | 13 | 35.028 | 49.062 | 49.786 | 1.00 | 10.82 | DIC |
| ATOM | 99 | N | TYR | 14 | 32.988 | 48.624 | 50.643 | 1.00 | 10.01 | DIC |
| ATOM | 100 | CA | TYR | 14 | 33.169 | 49.479 | 51.812 | 1.00 | 10.22 | DIC |
| ATOM | 101 | CB | TYR | 14 | 31.869 | 49.535 | 52.623 | 1.00 | 10.13 | DIC |
| ATOM | 102 | CG | TYR | 14 | 30.670 | 49.883 | 51.775 | 1.00 | 11.37 | DIC |
| ATOM | 103 | CD1 | TYR | 14 | 30.798 | 50.752 | 50.692 | 1.00 | 11.46 | DIC |
| ATOM | 104 | CE1 | TYR | 14 | 29.709 | 51.098 | 49.913 | 1.00 | 12.49 | DIC |
| ATOM | 105 | CD2 | TYR | 14 | 29.406 | 49.363 | 52.061 | 1.00 | 11.77 | DIC |
| ATOM | 106 | CE2 | TYR | 14 | 28.301 | 49.708 | 51.288 | 1.00 | 12.94 | DIC |
| ATOM | 107 | CZ | TYR | 14 | 28.464 | 50.581 | 50.211 | 1.00 | 13.37 | DIC |
| ATOM | 108 | OH | TYR | 14 | 27.387 | 50.969 | 49.445 | 1.00 | 13.68 | DIC |
| ATOM | 109 | C | TYR | 14 | 34.320 | 48.993 | 52.692 | 1.00 | 10.01 | DIC |
| ATOM | 110 | O | TYR | 14 | 35.101 | 49.796 | 53.209 | 1.00 | 10.44 | DIC |
| ATOM | 111 | N | LEU | 15 | 34.402 | 47.677 | 52.864 | 1.00 | 9.93 | DIC |
| ATOM | 112 | CA | LEU | 15 | 35.454 | 47.043 | 53.649 | 1.00 | 10.44 | DIC |
| ATOM | 113 | CB | LEU | 15 | 35.311 | 45.523 | 53.558 | 1.00 | 10.41 | DIC |
| ATOM | 114 | CG | LEU | 15 | 36.559 | 44.670 | 53.803 | 1.00 | 11.91 | DIC |
| ATOM | 115 | CD1 | LEU | 15 | 36.951 | 44.732 | 55.262 | 1.00 | 12.15 | DIC |
| ATOM | 116 | CD2 | LEU | 15 | 36.282 | 43.228 | 53.380 | 1.00 | 10.34 | DIC |
| ATOM | 117 | C | LEU | 15 | 36.822 | 47.451 | 53.097 | 1.00 | 9.78 | DIC |
| ATOM | 118 | O | LEU | 15 | 37.767 | 47.689 | 53.852 | 1.00 | 10.03 | DIC |
| ATOM | 119 | N | CYS | 16 | 36.917 | 47.505 | 51.771 | 1.00 | 9.90 | DIC |
| ATOM | 120 | CA | CYS | 16 | 38.160 | 47.878 | 51.111 | 1.00 | 9.59 | DIC |
| ATOM | 121 | CB | CYS | 16 | 38.005 | 47.761 | 49.590 | 1.00 | 9.35 | DIC |
| ATOM | 122 | SG | CYS | 16 | 39.528 | 48.155 | 48.711 | 1.00 | 10.55 | DIC |
| ATOM | 123 | C | CYS | 16 | 38.574 | 49.301 | 51.483 | 1.00 | 9.71 | DIC |
| ATOM | 124 | O | CYS | 16 | 39.737 | 49.564 | 51.793 | 1.00 | 10.85 | DIC |
| ATOM | 125 | N | VAL | 17 | 37.618 | 50.219 | 51.452 | 1.00 | 9.93 | DIC |
| ATOM | 126 | CA | VAL | 17 | 37.891 | 51.611 | 51.805 | 1.00 | 10.54 | DIC |
| ATOM | 127 | CB | VAL | 17 | 36.663 | 52.502 | 51.509 | 1.00 | 9.19 | DIC |
| ATOM | 128 | CG1 | VAL | 17 | 36.853 | 53.889 | 52.088 | 1.00 | 9.35 | DIC |
| ATOM | 129 | CG2 | VAL | 17 | 36.459 | 52.596 | 49.993 | 1.00 | 10.21 | DIC |
| ATOM | 130 | C | VAL | 17 | 38.277 | 51.730 | 53.281 | 1.00 | 11.16 | DIC |
| ATOM | 131 | O | VAL | 17 | 39.214 | 52.445 | 53.625 | 1.00 | 12.58 | DIC |
| ATOM | 132 | N | ALA | 18 | 37.562 | 51.031 | 54.154 | 1.00 | 11.08 | DIC |
| ATOM | 133 | CA | ALA | 18 | 37.877 | 51.094 | 55.580 | 1.00 | 10.23 | DIC |
| ATOM | 134 | CB | ALA | 18 | 36.838 | 50.319 | 56.390 | 1.00 | 9.79 | DIC |
| ATOM | 135 | C | ALA | 18 | 39.267 | 50.515 | 55.830 | 1.00 | 10.26 | DIC |
| ATOM | 136 | O | ALA | 18 | 40.059 | 51.075 | 56.598 | 1.00 | 9.16 | DIC |
| ATOM | 137 | N | ALA | 19 | 39.554 | 49.387 | 55.187 | 1.00 | 10.33 | DIC |
| ATOM | 138 | CA | ALA | 19 | 40.843 | 48.729 | 55.343 | 1.00 | 11.10 | DIC |
| ATOM | 139 | CB | ALA | 19 | 40.883 | 47.450 | 54.519 | 1.00 | 12.50 | DIC |
| ATOM | 140 | C | ALA | 19 | 41.955 | 49.672 | 54.910 | 1.00 | 12.35 | DIC |
| ATOM | 141 | O | ALA | 19 | 42.945 | 49.839 | 55.620 | 1.00 | 11.18 | DIC |
| ATOM | 142 | N | LYS | 20 | 41.786 | 50.307 | 53.755 | 1.00 | 12.66 | DIC |
| ATOM | 143 | CA | LYS | 20 | 42.812 | 51.227 | 53.277 | 1.00 | 13.17 | DIC |
| ATOM | 144 | CB | LYS | 20 | 42.485 | 51.723 | 51.872 | 1.00 | 14.36 | DIC |
| ATOM | 145 | CG | LYS | 20 | 43.592 | 52.576 | 51.284 | 1.00 | 18.06 | DIC |
| ATOM | 146 | CD | LYS | 20 | 43.350 | 52.878 | 49.815 | 1.00 | 21.04 | DIC |
| ATOM | 147 | CE | LYS | 20 | 44.518 | 53.666 | 49.229 | 1.00 | 22.73 | DIC |
| ATOM | 148 | NZ | LYS | 20 | 44.283 | 54.010 | 47.797 | 1.00 | 23.84 | DIC |
| ATOM | 149 | C | LYS | 20 | 42.994 | 52.410 | 54.227 | 1.00 | 12.86 | DIC |
| ATOM | 150 | O | LYS | 20 | 44.114 | 52.877 | 54.426 | 1.00 | 11.70 | DIC |
| ATOM | 151 | N | SER | 21 | 41.909 | 52.893 | 54.830 | 1.00 | 12.92 | DIC |
| ATOM | 152 | CA | SER | 21 | 42.049 | 54.014 | 55.759 | 1.00 | 13.27 | DIC |
| ATOM | 153 | CB | SER | 21 | 40.684 | 54.476 | 56.298 | 1.00 | 13.74 | DIC |
| ATOM | 154 | OG | SER | 21 | 40.217 | 53.672 | 57.370 | 1.00 | 13.06 | DIC |
| ATOM | 155 | C | SER | 21 | 42.964 | 53.580 | 56.904 | 1.00 | 12.88 | DIC |
| ATOM | 156 | O | SER | 21 | 43.782 | 54.360 | 57.383 | 1.00 | 13.01 | DIC |
| ATOM | 157 | N | VAL | 22 | 42.845 | 52.327 | 57.332 | 1.00 | 13.22 | DIC |
| ATOM | 158 | CA | VAL | 22 | 43.698 | 51.825 | 58.410 | 1.00 | 12.36 | DIC |
| ATOM | 159 | CB | VAL | 22 | 43.308 | 50.398 | 58.836 | 1.00 | 12.30 | DIC |
| ATOM | 160 | CG1 | VAL | 22 | 44.288 | 49.892 | 59.872 | 1.00 | 11.72 | DIC |
| ATOM | 161 | CG2 | VAL | 22 | 41.887 | 50.383 | 59.389 | 1.00 | 11.48 | DIC |
| ATOM | 162 | C | VAL | 22 | 45.164 | 51.799 | 57.979 | 1.00 | 12.37 | DIC |
| ATOM | 163 | O | VAL | 22 | 46.059 | 52.159 | 58.749 | 1.00 | 11.98 | DIC |
| ATOM | 164 | N | GLU | 23 | 45.420 | 51.364 | 56.751 | 1.00 | 12.92 | DIC |
| ATOM | 165 | CA | GLU | 23 | 46.800 | 51.317 | 56.276 | 1.00 | 13.21 | DIC |
| ATOM | 166 | CB | GLU | 23 | 46.890 | 50.655 | 54.897 | 1.00 | 13.48 | DIC |
| ATOM | 167 | CG | GLU | 23 | 46.489 | 49.186 | 54.863 | 1.00 | 14.09 | DIC |
| ATOM | 168 | CD | GLU | 23 | 46.831 | 48.546 | 53.536 | 1.00 | 14.75 | DIC |
| ATOM | 169 | OE1 | GLU | 23 | 46.676 | 49.224 | 52.503 | 1.00 | 14.77 | DIC |
| ATOM | 170 | OE2 | GLU | 23 | 47.247 | 47.369 | 53.519 | 1.00 | 14.64 | DIC |
| ATOM | 171 | C | GLU | 23 | 47.376 | 52.724 | 56.193 | 1.00 | 13.14 | DIC |
| ATOM | 172 | O | GLU | 23 | 48.504 | 52.961 | 56.615 | 1.00 | 13.52 | DIC |
| ATOM | 173 | N | ALA | 24 | 46.591 | 53.653 | 55.649 | 1.00 | 11.95 | DIC |
| ATOM | 174 | CA | ALA | 24 | 47.030 | 55.032 | 55.501 | 1.00 | 12.18 | DIC |
| ATOM | 175 | CB | ALA | 24 | 45.916 | 55.881 | 54.857 | 1.00 | 13.20 | DIC |
| ATOM | 176 | C | ALA | 24 | 47.433 | 55.636 | 56.833 | 1.00 | 11.58 | DIC |
| ATOM | 177 | O | ALA | 24 | 48.382 | 56.422 | 56.905 | 1.00 | 10.67 | DIC |
| ATOM | 178 | N | ALA | 25 | 46.722 | 55.259 | 57.891 | 1.00 | 11.32 | DIC |
| ATOM | 179 | CA | ALA | 25 | 46.994 | 55.784 | 59.225 | 1.00 | 11.21 | DIC |
| ATOM | 180 | CB | ALA | 25 | 45.745 | 55.648 | 60.096 | 1.00 | 10.62 | DIC |
| ATOM | 181 | C | ALA | 25 | 48.199 | 55.160 | 59.939 | 1.00 | 11.67 | DIC |
| ATOM | 182 | O | ALA | 25 | 48.613 | 55.651 | 60.985 | 1.00 | 11.24 | DIC |
| ATOM | 183 | N | HIS | 26 | 48.763 | 54.088 | 59.393 | 1.00 | 11.30 | DIC |
| ATOM | 184 | CA | HIS | 26 | 49.926 | 53.459 | 60.027 | 1.00 | 12.66 | DIC |
| ATOM | 185 | CB | HIS | 26 | 49.529 | 52.171 | 60.761 | 1.00 | 12.73 | DIC |
| ATOM | 186 | CG | HIS | 26 | 48.360 | 52.331 | 61.682 | 1.00 | 14.25 | DIC |
| ATOM | 187 | CD2 | HIS | 26 | 48.296 | 52.451 | 63.030 | 1.00 | 13.73 | DIC |
| ATOM | 188 | ND1 | HIS | 26 | 47.058 | 52.385 | 61.229 | 1.00 | 13.15 | DIC |
| ATOM | 189 | CE1 | HIS | 26 | 46.244 | 52.529 | 62.258 | 1.00 | 13.73 | DIC |
| ATOM | 190 | NE2 | HIS | 26 | 46.970 | 52.571 | 63.362 | 1.00 | 14.21 | DIC |
| ATOM | 191 | C | HIS | 26 | 50.968 | 53.126 | 58.965 | 1.00 | 12.72 | DIC |
| ATOM | 192 | O | HIS | 26 | 51.201 | 51.956 | 58.654 | 1.00 | 12.84 | DIC |
| ATOM | 193 | N | PRO | 27 | 51.616 | 54.155 | 58.402 | 1.00 | 13.48 | DIC |
| ATOM | 194 | CD | PRO | 27 | 51.482 | 55.586 | 58.732 | 1.00 | 14.47 | DIC |
| ATOM | 195 | CA | PRO | 27 | 52.628 | 53.949 | 57.369 | 1.00 | 14.03 | DIC |
| ATOM | 196 | CB | PRO | 27 | 53.064 | 55.377 | 57.015 | 1.00 | 14.64 | DIC |
| ATOM | 197 | CG | PRO | 27 | 52.802 | 56.148 | 58.271 | 1.00 | 15.02 | DIC |
| ATOM | 198 | C | PRO | 27 | 53.800 | 53.040 | 57.730 | 1.00 | 13.73 | DIC |
| ATOM | 199 | O | PRO | 27 | 54.418 | 52.447 | 56.842 | 1.00 | 14.16 | DIC |
| ATOM | 200 | N | ASP | 28 | 54.119 | 52.912 | 59.013 | 1.00 | 13.19 | DIC |
| ATOM | 201 | CA | ASP | 28 | 55.240 | 52.044 | 59.368 | 1.00 | 13.54 | DIC |
| ATOM | 202 | CB | ASP | 28 | 56.407 | 52.855 | 59.934 | 1.00 | 14.01 | DIC |
| ATOM | 203 | CG | ASP | 28 | 57.687 | 52.033 | 60.027 | 1.00 | 12.49 | DIC |
| ATOM | 204 | OD1 | ASP | 28 | 57.987 | 51.294 | 59.066 | 1.00 | 13.79 | DIC |
| ATOM | 205 | OD2 | ASP | 28 | 58.390 | 52.123 | 61.047 | 1.00 | 11.67 | DIC |
| ATOM | 206 | C | ASP | 28 | 54.883 | 50.930 | 60.335 | 1.00 | 14.24 | DIC |
| ATOM | 207 | O | ASP | 28 | 55.706 | 50.504 | 61.146 | 1.00 | 13.51 | DIC |
| ATOM | 208 | N | THR | 29 | 53.651 | 50.450 | 60.242 | 1.00 | 14.30 | DIC |
| ATOM | 209 | CA | THR | 29 | 53.219 | 49.367 | 61.102 | 1.00 | 15.03 | DIC |
| ATOM | 210 | CB | THR | 29 | 52.156 | 49.818 | 62.104 | 1.00 | 13.26 | DIC |
| ATOM | 211 | OG1 | THR | 29 | 52.632 | 50.956 | 62.828 | 1.00 | 12.77 | DIC |
| ATOM | 212 | CG2 | THR | 29 | 51.865 | 48.694 | 63.090 | 1.00 | 13.03 | DIC |
| ATOM | 213 | C | THR | 29 | 52.636 | 48.266 | 60.243 | 1.00 | 15.57 | DIC |
| ATOM | 214 | O | THR | 29 | 51.882 | 48.528 | 59.305 | 1.00 | 14.98 | DIC |
| ATOM | 215 | N | GLU | 30 | 53.012 | 47.034 | 60.554 | 1.00 | 16.16 | DIC |
| ATOM | 216 | CA | GLU | 30 | 52.520 | 45.874 | 59.825 | 1.00 | 17.32 | DIC |
| ATOM | 217 | CB | GLU | 30 | 53.287 | 44.630 | 60.281 | 1.00 | 19.02 | DIC |
| ATOM | 218 | CG | GLU | 30 | 53.694 | 43.659 | 59.178 | 1.00 | 23.03 | DIC |
| ATOM | 219 | CD | GLU | 30 | 54.316 | 44.344 | 57.969 | 1.00 | 23.93 | DIC |
| ATOM | 220 | OE1 | GLU | 30 | 53.564 | 44.698 | 57.039 | 1.00 | 24.55 | DIC |
| ATOM | 221 | OE2 | GLU | 30 | 55.549 | 44.537 | 57.947 | 1.00 | 24.76 | DIC |
| ATOM | 222 | C | GLU | 30 | 51.046 | 45.750 | 60.185 | 1.00 | 16.48 | DIC |
| ATOM | 223 | O | GLU | 30 | 50.701 | 45.675 | 61.368 | 1.00 | 16.29 | DIC |
| ATOM | 224 | N | ILE | 31 | 50.174 | 45.760 | 59.181 | 1.00 | 15.30 | DIC |
| ATOM | 225 | CA | ILE | 31 | 48.743 | 45.628 | 59.436 | 1.00 | 15.17 | DIC |
| ATOM | 226 | CB | ILE | 31 | 47.905 | 46.770 | 58.778 | 1.00 | 14.27 | DIC |
| ATOM | 227 | CG2 | ILE | 31 | 46.420 | 46.573 | 59.106 | 1.00 | 14.26 | DIC |
| ATOM | 228 | CG1 | ILE | 31 | 48.368 | 48.140 | 59.277 | 1.00 | 13.70 | DIC |
| ATOM | 229 | CD | ILE | 31 | 48.085 | 48.397 | 60.740 | 1.00 | 12.11 | DIC |
| ATOM | 230 | C | ILE | 31 | 48.245 | 44.305 | 58.868 | 1.00 | 14.65 | DIC |
| ATOM | 231 | O | ILE | 31 | 48.185 | 44.127 | 57.650 | 1.00 | 14.91 | DIC |
| ATOM | 232 | N | ARG | 32 | 47.891 | 43.384 | 59.757 | 1.00 | 13.99 | DIC |
| ATOM | 233 | CA | ARG | 32 | 47.384 | 42.087 | 59.349 | 1.00 | 13.79 | DIC |
| ATOM | 234 | CB | ARG | 32 | 47.983 | 40.982 | 60.225 | 1.00 | 15.26 | DIC |
| ATOM | 235 | CG | ARG | 32 | 49.510 | 40.934 | 60.177 | 1.00 | 18.70 | DIC |
| ATOM | 236 | CD | ARG | 32 | 50.067 | 39.639 | 60.752 | 1.00 | 21.47 | DIC |
| ATOM | 237 | NE | ARG | 32 | 49.766 | 39.471 | 62.173 | 1.00 | 24.86 | DIC |
| ATOM | 238 | CZ | ARG | 32 | 50.180 | 38.439 | 62.905 | 1.00 | 27.54 | DIC |
| ATOM | 239 | NH1 | ARG | 32 | 50.916 | 37.483 | 62.346 | 1.00 | 28.88 | DIC |
| ATOM | 240 | NH2 | ARG | 32 | 49.861 | 38.360 | 64.196 | 1.00 | 27.69 | DIC |
| ATOM | 241 | C | ARG | 32 | 45.867 | 42.107 | 59.478 | 1.00 | 13.24 | DIC |
| ATOM | 242 | O | ARG | 32 | 45.330 | 42.234 | 60.578 | 1.00 | 11.50 | DIC |
| ATOM | 243 | N | PHE | 33 | 45.185 | 41.997 | 58.341 | 1.00 | 12.52 | DIC |
| ATOM | 244 | CA | PHE | 33 | 43.730 | 42.009 | 58.301 | 1.00 | 11.78 | DIC |
| ATOM | 245 | CB | PHE | 33 | 43.246 | 42.686 | 57.010 | 1.00 | 10.75 | DIC |
| ATOM | 246 | CG | PHE | 33 | 43.502 | 44.171 | 56.950 | 1.00 | 9.65 | DIC |
| ATOM | 247 | CD1 | PHE | 33 | 42.777 | 45.054 | 57.748 | 1.00 | 9.25 | DIC |
| ATOM | 248 | CD2 | PHE | 33 | 44.414 | 44.693 | 56.038 | 1.00 | 10.53 | DIC |
| ATOM | 249 | CE1 | PHE | 33 | 42.953 | 46.436 | 57.627 | 1.00 | 9.15 | DIC |
| ATOM | 250 | CE2 | PHE | 33 | 44.600 | 46.077 | 55.908 | 1.00 | 9.49 | DIC |
| ATOM | 251 | CZ | PHE | 33 | 43.864 | 46.944 | 56.703 | 1.00 | 9.68 | DIC |
| ATOM | 252 | C | PHE | 33 | 43.134 | 40.597 | 58.365 | 1.00 | 11.96 | DIC |
| ATOM | 253 | O | PHE | 33 | 43.565 | 39.697 | 57.639 | 1.00 | 12.74 | DIC |
| ATOM | 254 | N | HIS | 34 | 42.145 | 40.420 | 59.238 | 1.00 | 11.46 | DIC |
| ATOM | 255 | CA | HIS | 34 | 41.431 | 39.149 | 59.403 | 1.00 | 11.17 | DIC |
| ATOM | 256 | CB | HIS | 34 | 41.523 | 38.659 | 60.850 | 1.00 | 11.57 | DIC |
| ATOM | 257 | CG | HIS | 34 | 42.919 | 38.593 | 61.384 | 1.00 | 12.66 | DIC |
| ATOM | 258 | CD2 | HIS | 34 | 43.704 | 39.544 | 61.942 | 1.00 | 13.37 | DIC |
| ATOM | 259 | ND1 | HIS | 34 | 43.667 | 37.437 | 61.370 | 1.00 | 13.22 | DIC |
| ATOM | 260 | CE1 | HIS | 34 | 44.854 | 37.677 | 61.899 | 1.00 | 13.27 | DIC |
| ATOM | 261 | NE2 | HIS | 34 | 44.903 | 38.947 | 62.255 | 1.00 | 14.11 | DIC |
| ATOM | 262 | C | HIS | 34 | 39.971 | 39.480 | 59.091 | 1.00 | 11.20 | DIC |
| ATOM | 263 | O | HIS | 34 | 39.336 | 40.217 | 59.830 | 1.00 | 9.79 | DIC |
| ATOM | 264 | N | VAL | 35 | 39.428 | 38.944 | 58.010 | 1.00 | 10.16 | DIC |
| ATOM | 265 | CA | VAL | 35 | 38.050 | 39.266 | 57.679 | 1.00 | 10.38 | DIC |
| ATOM | 266 | CB | VAL | 35 | 37.945 | 39.733 | 56.208 | 1.00 | 11.51 | DIC |
| ATOM | 267 | CG1 | VAL | 35 | 36.512 | 40.077 | 55.867 | 1.00 | 11.42 | DIC |
| ATOM | 268 | CG2 | VAL | 35 | 38.847 | 40.945 | 55.985 | 1.00 | 12.17 | DIC |
| ATOM | 269 | C | VAL | 35 | 37.072 | 38.126 | 57.929 | 1.00 | 10.51 | DIC |
| ATOM | 270 | O | VAL | 35 | 37.240 | 37.017 | 57.408 | 1.00 | 9.02 | DIC |
| ATOM | 271 | N | LEU | 36 | 36.060 | 38.397 | 58.754 | 1.00 | 10.05 | DIC |
| ATOM | 272 | CA | LEU | 36 | 35.027 | 37.412 | 59.044 | 1.00 | 10.98 | DIC |
| ATOM | 273 | CB | LEU | 36 | 34.282 | 37.766 | 60.341 | 1.00 | 11.52 | DIC |
| ATOM | 274 | CG | LEU | 36 | 35.176 | 37.835 | 61.592 | 1.00 | 12.34 | DIC |
| ATOM | 275 | CD1 | LEU | 36 | 34.319 | 37.965 | 62.858 | 1.00 | 13.53 | DIC |
| ATOM | 276 | CD2 | LEU | 36 | 36.035 | 36.574 | 61.679 | 1.00 | 13.20 | DIC |
| ATOM | 277 | C | LEU | 36 | 34.123 | 37.527 | 57.821 | 1.00 | 11.61 | DIC |
| ATOM | 278 | O | LEU | 36 | 33.275 | 38.409 | 57.728 | 1.00 | 10.93 | DIC |
| ATOM | 279 | N | ASP | 37 | 34.352 | 36.620 | 56.881 | 1.00 | 12.50 | DIC |
| ATOM | 280 | CA | ASP | 37 | 33.678 | 36.580 | 55.591 | 1.00 | 13.19 | DIC |
| ATOM | 281 | CB | ASP | 37 | 34.660 | 35.981 | 54.580 | 1.00 | 13.28 | DIC |
| ATOM | 282 | CG | ASP | 37 | 34.090 | 35.878 | 53.189 | 1.00 | 13.48 | DIC |
| ATOM | 283 | OD1 | ASP | 37 | 32.903 | 36.212 | 52.997 | 1.00 | 14.14 | DIC |
| ATOM | 284 | OD2 | ASP | 37 | 34.843 | 35.456 | 52.290 | 1.00 | 13.50 | DIC |
| ATOM | 285 | C | ASP | 37 | 32.369 | 35.803 | 55.588 | 1.00 | 14.68 | DIC |
| ATOM | 286 | O | ASP | 37 | 32.363 | 34.569 | 55.634 | 1.00 | 14.18 | DIC |
| ATOM | 287 | N | ALA | 38 | 31.258 | 36.526 | 55.511 | 1.00 | 14.79 | DIC |
| ATOM | 288 | CA | ALA | 38 | 29.954 | 35.884 | 55.521 | 1.00 | 15.72 | DIC |
| ATOM | 289 | CB | ALA | 38 | 28.983 | 36.689 | 56.390 | 1.00 | 16.90 | DIC |
| ATOM | 290 | C | ALA | 38 | 29.362 | 35.669 | 54.132 | 1.00 | 15.90 | DIC |
| ATOM | 291 | O | ALA | 38 | 28.143 | 35.600 | 53.978 | 1.00 | 17.34 | DIC |
| ATOM | 292 | N | GLY | 39 | 30.213 | 35.557 | 53.120 | 1.00 | 15.36 | DIC |
| ATOM | 293 | CA | GLY | 39 | 29.698 | 35.321 | 51.786 | 1.00 | 13.98 | DIC |
| ATOM | 294 | C | GLY | 39 | 30.208 | 36.249 | 50.706 | 1.00 | 12.66 | DIC |
| ATOM | 295 | O | GLY | 39 | 29.534 | 36.454 | 49.701 | 1.00 | 13.26 | DIC |
| ATOM | 296 | N | ILE | 40 | 31.395 | 36.805 | 50.901 | 1.00 | 11.81 | DIC |
| ATOM | 297 | CA | ILE | 40 | 31.985 | 37.695 | 49.909 | 1.00 | 12.00 | DIC |
| ATOM | 298 | CB | ILE | 40 | 33.220 | 38.417 | 50.493 | 1.00 | 11.76 | DIC |
| ATOM | 299 | CG2 | ILE | 40 | 33.764 | 39.419 | 49.488 | 1.00 | 12.77 | DIC |
| ATOM | 300 | CG1 | ILE | 40 | 32.815 | 39.178 | 51.761 | 1.00 | 10.15 | DIC |
| ATOM | 301 | CD | ILE | 40 | 33.981 | 39.741 | 52.549 | 1.00 | 10.52 | DIC |
| ATOM | 302 | C | ILE | 40 | 32.389 | 36.857 | 48.688 | 1.00 | 12.84 | DIC |
| ATOM | 303 | O | ILE | 40 | 32.958 | 35.772 | 48.828 | 1.00 | 12.10 | DIC |
| ATOM | 304 | N | SER | 41 | 32.078 | 37.350 | 47.491 | 1.00 | 13.34 | DIC |
| ATOM | 305 | CA | SER | 41 | 32.410 | 36.629 | 46.265 | 1.00 | 13.44 | DIC |
| ATOM | 306 | CB | SER | 41 | 31.778 | 37.311 | 45.043 | 1.00 | 12.76 | DIC |
| ATOM | 307 | OG | SER | 41 | 32.427 | 38.536 | 44.747 | 1.00 | 12.89 | DIC |
| ATOM | 308 | C | SER | 41 | 33.909 | 36.552 | 46.055 | 1.00 | 14.12 | DIC |
| ATOM | 309 | O | SER | 41 | 34.662 | 37.369 | 46.583 | 1.00 | 14.41 | DIC |
| ATOM | 310 | N | GLU | 42 | 34.336 | 35.570 | 45.267 | 1.00 | 15.57 | DIC |
| ATOM | 311 | CA | GLU | 42 | 35.749 | 35.401 | 44.968 | 1.00 | 16.08 | DIC |
| ATOM | 312 | CB | GLU | 42 | 35.967 | 34.183 | 44.060 | 1.00 | 17.55 | DIC |
| ATOM | 313 | CG | GLU | 42 | 37.424 | 33.922 | 43.704 | 1.00 | 19.96 | DIC |
| ATOM | 314 | CD | GLU | 42 | 37.961 | 34.853 | 42.631 | 1.00 | 21.69 | DIC |
| ATOM | 315 | OE1 | GLU | 42 | 39.199 | 35.047 | 42.572 | 1.00 | 23.34 | DIC |
| ATOM | 316 | OE2 | GLU | 42 | 37.153 | 35.382 | 41.835 | 1.00 | 22.79 | DIC |
| ATOM | 317 | C | GLU | 42 | 36.229 | 36.660 | 44.265 | 1.00 | 15.85 | DIC |
| ATOM | 318 | O | GLU | 42 | 37.332 | 37.137 | 44.511 | 1.00 | 15.67 | DIC |
| ATOM | 319 | N | ALA | 43 | 35.385 | 37.198 | 43.393 | 1.00 | 14.84 | DIC |
| ATOM | 320 | CA | ALA | 43 | 35.725 | 38.399 | 42.654 | 1.00 | 15.14 | DIC |
| ATOM | 321 | CB | ALA | 43 | 34.635 | 38.710 | 41.622 | 1.00 | 15.42 | DIC |
| ATOM | 322 | C | ALA | 43 | 35.907 | 39.579 | 43.600 | 1.00 | 14.89 | DIC |
| ATOM | 323 | O | ALA | 43 | 36.821 | 40.381 | 43.430 | 1.00 | 14.31 | DIC |
| ATOM | 324 | N | ASN | 44 | 35.048 | 39.683 | 44.610 | 1.00 | 15.08 | DIC |
| ATOM | 325 | CA | ASN | 44 | 35.163 | 40.794 | 45.550 | 1.00 | 14.76 | DIC |
| ATOM | 326 | CB | ASN | 44 | 33.847 | 41.008 | 46.304 | 1.00 | 14.30 | DIC |
| ATOM | 327 | CG | ASN | 44 | 32.817 | 41.746 | 45.464 | 1.00 | 15.33 | DIC |
| ATOM | 328 | OD1 | ASN | 44 | 33.167 | 42.625 | 44.674 | 1.00 | 14.17 | DIC |
| ATOM | 329 | ND2 | ASN | 44 | 31.543 | 41.406 | 45.637 | 1.00 | 15.84 | DIC |
| ATOM | 330 | C | ASN | 44 | 36.324 | 40.661 | 46.529 | 1.00 | 14.57 | DIC |
| ATOM | 331 | O | ASN | 44 | 36.852 | 41.666 | 47.005 | 1.00 | 14.31 | DIC |
| ATOM | 332 | N | ARG | 45 | 36.732 | 39.431 | 46.828 | 1.00 | 14.77 | DIC |
| ATOM | 333 | CA | ARG | 45 | 37.853 | 39.229 | 47.739 | 1.00 | 15.32 | DIC |
| ATOM | 334 | CB | ARG | 45 | 37.926 | 37.771 | 48.205 | 1.00 | 16.87 | DIC |
| ATOM | 335 | CG | ARG | 45 | 36.589 | 37.242 | 48.689 | 1.00 | 20.54 | DIC |
| ATOM | 336 | CD | ARG | 45 | 36.710 | 36.377 | 49.924 | 1.00 | 24.39 | DIC |
| ATOM | 337 | NE | ARG | 45 | 37.571 | 35.215 | 49.725 | 1.00 | 27.75 | DIC |
| ATOM | 338 | CZ | ARG | 45 | 37.704 | 34.225 | 50.608 | 1.00 | 29.60 | DIC |
| ATOM | 339 | NH1 | ARG | 45 | 37.028 | 34.247 | 51.752 | 1.00 | 30.56 | DIC |
| ATOM | 340 | NH2 | ARG | 45 | 38.526 | 33.216 | 50.353 | 1.00 | 30.76 | DIC |
| ATOM | 341 | C | ARG | 45 | 39.141 | 39.608 | 47.020 | 1.00 | 15.22 | DIC |
| ATOM | 342 | O | ARG | 45 | 40.002 | 40.277 | 47.590 | 1.00 | 14.16 | DIC |
| ATOM | 343 | N | ALA | 46 | 39.265 | 39.194 | 45.761 | 1.00 | 14.15 | DIC |
| ATOM | 344 | CA | ALA | 46 | 40.458 | 39.515 | 44.983 | 1.00 | 14.85 | DIC |
| ATOM | 345 | CB | ALA | 46 | 40.395 | 38.840 | 43.610 | 1.00 | 15.30 | DIC |
| ATOM | 346 | C | ALA | 46 | 40.545 | 41.029 | 44.823 | 1.00 | 14.64 | DIC |
| ATOM | 347 | O | ALA | 46 | 41.623 | 41.616 | 44.910 | 1.00 | 15.26 | DIC |
| ATOM | 348 | N | ALA | 47 | 39.397 | 41.662 | 44.600 | 1.00 | 14.80 | DIC |
| ATOM | 349 | CA | ALA | 47 | 39.348 | 43.112 | 44.430 | 1.00 | 14.02 | DIC |
| ATOM | 350 | CB | ALA | 47 | 37.938 | 43.536 | 44.039 | 1.00 | 15.12 | DIC |
| ATOM | 351 | C | ALA | 47 | 39.795 | 43.857 | 45.693 | 1.00 | 13.42 | DIC |
| ATOM | 352 | O | ALA | 47 | 40.562 | 44.823 | 45.618 | 1.00 | 12.41 | DIC |
| ATOM | 353 | N | VAL | 48 | 39.316 | 43.415 | 46.853 | 1.00 | 13.25 | DIC |
| ATOM | 354 | CA | VAL | 48 | 39.694 | 44.052 | 48.111 | 1.00 | 12.62 | DIC |
| ATOM | 355 | CB | VAL | 48 | 38.922 | 43.445 | 49.305 | 1.00 | 11.98 | DIC |
| ATOM | 356 | CG1 | VAL | 48 | 39.458 | 43.992 | 50.609 | 1.00 | 10.86 | DIC |
| ATOM | 357 | CG2 | VAL | 48 | 37.460 | 43.786 | 49.193 | 1.00 | 11.68 | DIC |
| ATOM | 358 | C | VAL | 48 | 41.192 | 43.891 | 48.350 | 1.00 | 12.98 | DIC |
| ATOM | 359 | O | VAL | 48 | 41.884 | 44.855 | 48.682 | 1.00 | 12.57 | DIC |
| ATOM | 360 | N | ALA | 49 | 41.692 | 42.672 | 48.168 | 1.00 | 13.13 | DIC |
| ATOM | 361 | CA | ALA | 49 | 43.102 | 42.386 | 48.374 | 1.00 | 14.18 | DIC |
| ATOM | 362 | CB | ALA | 49 | 43.355 | 40.892 | 48.245 | 1.00 | 13.51 | DIC |
| ATOM | 363 | C | ALA | 49 | 43.983 | 43.145 | 47.397 | 1.00 | 14.36 | DIC |
| ATOM | 364 | O | ALA | 49 | 45.072 | 43.601 | 47.752 | 1.00 | 15.03 | DIC |
| ATOM | 365 | N | ALA | 50 | 43.519 | 43.274 | 46.162 | 1.00 | 15.04 | DIC |
| ATOM | 366 | CA | ALA | 50 | 44.290 | 43.979 | 45.142 | 1.00 | 15.89 | DIC |
| ATOM | 367 | CB | ALA | 50 | 43.582 | 43.882 | 43.792 | 1.00 | 16.15 | DIC |
| ATOM | 368 | C | ALA | 50 | 44.530 | 45.443 | 45.492 | 1.00 | 16.46 | DIC |
| ATOM | 369 | O | ALA | 50 | 45.515 | 46.039 | 45.052 | 1.00 | 17.84 | DIC |
| ATOM | 370 | N | ASN | 51 | 43.631 | 46.029 | 46.277 | 1.00 | 16.90 | DIC |
| ATOM | 371 | CA | ASN | 51 | 43.758 | 47.432 | 46.661 | 1.00 | 17.24 | DIC |
| ATOM | 372 | CB | ASN | 51 | 42.379 | 48.060 | 46.826 | 1.00 | 17.14 | DIC |
| ATOM | 373 | CG | ASN | 51 | 41.709 | 48.355 | 45.507 | 1.00 | 16.69 | DIC |
| ATOM | 374 | OD1 | ASN | 51 | 42.190 | 49.172 | 44.729 | 1.00 | 18.27 | DIC |
| ATOM | 375 | ND2 | ASN | 51 | 40.584 | 47.700 | 45.253 | 1.00 | 16.79 | DIC |
| ATOM | 376 | C | ASN | 51 | 44.545 | 47.667 | 47.942 | 1.00 | 18.59 | DIC |
| ATOM | 377 | O | ASN | 51 | 44.784 | 48.815 | 48.324 | 1.00 | 17.91 | DIC |
| ATOM | 378 | N | LEU | 52 | 44.946 | 46.595 | 48.613 | 1.00 | 19.74 | DIC |
| ATOM | 379 | CA | LEU | 52 | 45.689 | 46.750 | 49.860 | 1.00 | 21.37 | DIC |
| ATOM | 380 | CB | LEU | 52 | 45.316 | 45.636 | 50.834 | 1.00 | 20.31 | DIC |
| ATOM | 381 | CG | LEU | 52 | 43.808 | 45.589 | 51.097 | 1.00 | 19.14 | DIC |
| ATOM | 382 | CD1 | LEU | 52 | 43.488 | 44.439 | 52.019 | 1.00 | 19.58 | DIC |
| ATOM | 383 | CD2 | LEU | 52 | 43.344 | 46.896 | 51.694 | 1.00 | 19.21 | DIC |
| ATOM | 384 | C | LEU | 52 | 47.195 | 46.798 | 49.645 | 1.00 | 23.35 | DIC |
| ATOM | 385 | O | LEU | 52 | 47.703 | 46.358 | 48.611 | 1.00 | 23.99 | DIC |
| ATOM | 386 | N | ARG | 53 | 47.896 | 47.337 | 50.639 | 1.00 | 25.01 | DIC |
| ATOM | 387 | CA | ARG | 53 | 49.346 | 47.509 | 50.589 | 1.00 | 27.42 | DIC |
| ATOM | 388 | CB | ARG | 53 | 49.894 | 47.695 | 52.012 | 1.00 | 26.42 | DIC |
| ATOM | 389 | CG | ARG | 53 | 50.983 | 48.739 | 52.099 | 1.00 | 25.31 | DIC |
| ATOM | 390 | CD | ARG | 53 | 50.808 | 49.649 | 53.310 | 1.00 | 24.31 | DIC |
| ATOM | 391 | NE | ARG | 53 | 50.903 | 48.918 | 54.567 | 1.00 | 22.35 | DIC |
| ATOM | 392 | CZ | ARG | 53 | 50.893 | 49.492 | 55.766 | 1.00 | 21.59 | DIC |
| ATOM | 393 | NH1 | ARG | 53 | 50.791 | 50.811 | 55.878 | 1.00 | 20.50 | DIC |
| ATOM | 394 | NH2 | ARG | 53 | 50.987 | 48.741 | 56.854 | 1.00 | 20.22 | DIC |
| ATOM | 395 | C | ARG | 53 | 50.089 | 46.390 | 49.875 | 1.00 | 28.67 | DIC |
| ATOM | 396 | O | ARG | 53 | 50.483 | 46.536 | 48.717 | 1.00 | 29.49 | DIC |
| ATOM | 397 | N | GLY | 55 | 50.292 | 45.278 | 50.563 | 1.00 | 30.50 | DIC |
| ATOM | 398 | CA | GLY | 55 | 50.987 | 44.173 | 49.937 | 1.00 | 32.29 | DIC |
| ATOM | 399 | C | GLY | 55 | 49.974 | 43.171 | 49.431 | 1.00 | 33.53 | DIC |
| ATOM | 400 | O | GLY | 55 | 50.312 | 42.264 | 48.663 | 1.00 | 34.18 | DIC |
| ATOM | 401 | N | GLY | 56 | 48.724 | 43.351 | 49.857 | 1.00 | 33.60 | DIC |
| ATOM | 402 | CA | GLY | 56 | 47.659 | 42.445 | 49.462 | 1.00 | 33.15 | DIC |
| ATOM | 403 | C | GLY | 56 | 47.872 | 41.107 | 50.146 | 1.00 | 33.10 | DIC |
| ATOM | 404 | O | GLY | 56 | 46.962 | 40.273 | 50.215 | 1.00 | 33.20 | DIC |
| ATOM | 405 | N | GLY | 57 | 49.088 | 40.914 | 50.656 | 1.00 | 32.04 | DIC |
| ATOM | 406 | CA | GLY | 57 | 49.447 | 39.682 | 51.333 | 1.00 | 30.83 | DIC |
| ATOM | 407 | C | GLY | 57 | 49.086 | 39.682 | 52.804 | 1.00 | 29.47 | DIC |
| ATOM | 408 | O | GLY | 57 | 49.138 | 38.640 | 53.457 | 1.00 | 30.37 | DIC |
| ATOM | 409 | N | ASN | 58 | 48.726 | 40.843 | 53.341 | 1.00 | 28.08 | DIC |
| ATOM | 410 | CA | ASN | 58 | 48.353 | 40.905 | 54.747 | 1.00 | 26.03 | DIC |
| ATOM | 411 | CB | ASN | 58 | 48.987 | 42.120 | 55.427 | 1.00 | 27.85 | DIC |
| ATOM | 412 | CG | ASN | 58 | 50.322 | 41.789 | 56.080 | 1.00 | 29.56 | DIC |
| ATOM | 413 | OD1 | ASN | 58 | 50.447 | 40.787 | 56.791 | 1.00 | 30.26 | DIC |
| ATOM | 414 | ND2 | ASN | 58 | 51.325 | 42.635 | 55.852 | 1.00 | 30.45 | DIC |
| ATOM | 415 | C | ASN | 58 | 46.845 | 40.893 | 54.995 | 1.00 | 24.13 | DIC |
| ATOM | 416 | O | ASN | 58 | 46.347 | 41.597 | 55.872 | 1.00 | 23.13 | DIC |
| ATOM | 417 | N | ILE | 59 | 46.114 | 40.099 | 54.217 | 1.00 | 20.72 | DIC |
| ATOM | 418 | CA | ILE | 59 | 44.678 | 39.992 | 54.423 | 1.00 | 19.23 | DIC |
| ATOM | 419 | CB | ILE | 59 | 43.873 | 40.915 | 53.471 | 1.00 | 19.07 | DIC |
| ATOM | 420 | CG2 | ILE | 59 | 44.100 | 40.511 | 52.016 | 1.00 | 18.83 | DIC |
| ATOM | 421 | CG1 | ILE | 59 | 42.387 | 40.848 | 53.838 | 1.00 | 18.98 | DIC |
| ATOM | 422 | CD | ILE | 59 | 41.510 | 41.865 | 53.110 | 1.00 | 19.26 | DIC |
| ATOM | 423 | C | ILE | 59 | 44.213 | 38.551 | 54.259 | 1.00 | 18.23 | DIC |
| ATOM | 424 | O | ILE | 59 | 44.446 | 37.916 | 53.233 | 1.00 | 17.66 | DIC |
| ATOM | 425 | N | ARG | 60 | 43.561 | 38.035 | 55.289 | 1.00 | 17.86 | DIC |
| ATOM | 426 | CA | ARG | 60 | 43.065 | 36.667 | 55.267 | 1.00 | 17.16 | DIC |
| ATOM | 427 | CB | ARG | 60 | 43.768 | 35.845 | 56.355 | 1.00 | 18.42 | DIC |
| ATOM | 428 | CG | ARG | 60 | 43.308 | 34.401 | 56.461 | 1.00 | 21.63 | DIC |
| ATOM | 429 | CD | ARG | 60 | 44.434 | 33.455 | 56.908 | 1.00 | 25.56 | DIC |
| ATOM | 430 | NE | ARG | 60 | 44.956 | 33.771 | 58.234 | 1.00 | 29.10 | DIC |
| ATOM | 431 | CZ | ARG | 60 | 45.878 | 34.698 | 58.483 | 1.00 | 31.08 | DIC |
| ATOM | 432 | NH1 | ARG | 60 | 46.397 | 35.411 | 57.489 | 1.00 | 32.55 | DIC |
| ATOM | 433 | NH2 | ARG | 60 | 46.277 | 34.922 | 59.730 | 1.00 | 31.29 | DIC |
| ATOM | 434 | C | ARG | 60 | 41.564 | 36.668 | 55.491 | 1.00 | 16.93 | DIC |
| ATOM | 435 | O | ARG | 60 | 41.067 | 37.309 | 56.422 | 1.00 | 16.37 | DIC |
| ATOM | 436 | N | PHE | 61 | 40.834 | 35.967 | 54.629 | 1.00 | 14.83 | DIC |
| ATOM | 437 | CA | PHE | 61 | 39.391 | 35.895 | 54.777 | 1.00 | 15.90 | DIC |
| ATOM | 438 | CB | PHE | 61 | 38.705 | 35.931 | 53.413 | 1.00 | 14.84 | DIC |
| ATOM | 439 | CG | PHE | 61 | 38.919 | 37.215 | 52.670 | 1.00 | 13.10 | DIC |
| ATOM | 440 | CD1 | PHE | 61 | 40.034 | 37.389 | 51.862 | 1.00 | 14.08 | DIC |
| ATOM | 441 | CD2 | PHE | 61 | 38.010 | 38.258 | 52.793 | 1.00 | 12.89 | DIC |
| ATOM | 442 | CE1 | PHE | 61 | 40.241 | 38.585 | 51.185 | 1.00 | 13.50 | DIC |
| ATOM | 443 | CE2 | PHE | 61 | 38.206 | 39.457 | 52.121 | 1.00 | 12.60 | DIC |
| ATOM | 444 | CZ | PHE | 61 | 39.322 | 39.623 | 51.316 | 1.00 | 13.60 | DIC |
| ATOM | 445 | C | PHE | 61 | 39.022 | 34.624 | 55.520 | 1.00 | 15.95 | DIC |
| ATOM | 446 | O | PHE | 61 | 39.541 | 33.555 | 55.221 | 1.00 | 15.87 | DIC |
| ATOM | 447 | N | ILE | 62 | 38.131 | 34.750 | 56.498 | 1.00 | 16.48 | DIC |
| ATOM | 448 | CA | ILE | 62 | 37.709 | 33.611 | 57.304 | 1.00 | 16.08 | DIC |
| ATOM | 449 | CB | ILE | 62 | 37.979 | 33.864 | 58.801 | 1.00 | 15.26 | DIC |
| ATOM | 450 | CG2 | ILE | 62 | 37.698 | 32.604 | 59.611 | 1.00 | 13.49 | DIC |
| ATOM | 451 | CG1 | ILE | 62 | 39.428 | 34.292 | 59.002 | 1.00 | 14.70 | DIC |
| ATOM | 452 | CD | ILE | 62 | 39.692 | 34.939 | 60.347 | 1.00 | 14.47 | DIC |
| ATOM | 453 | C | ILE | 62 | 36.220 | 33.360 | 57.123 | 1.00 | 17.10 | DIC |
| ATOM | 454 | O | ILE | 62 | 35.385 | 34.154 | 57.558 | 1.00 | 16.19 | DIC |
| ATOM | 455 | N | ASP | 63 | 35.895 | 32.242 | 56.489 | 1.00 | 18.05 | DIC |
| ATOM | 456 | CA | ASP | 63 | 34.511 | 31.883 | 56.245 | 1.00 | 18.87 | DIC |
| ATOM | 457 | CB | ASP | 63 | 34.451 | 30.589 | 55.420 | 1.00 | 21.18 | DIC |
| ATOM | 458 | CG | ASP | 63 | 34.653 | 30.834 | 53.932 | 1.00 | 22.82 | DIC |
| ATOM | 459 | OD1 | ASP | 63 | 35.479 | 31.697 | 53.565 | 1.00 | 23.93 | DIC |
| ATOM | 460 | OD2 | ASP | 63 | 33.986 | 30.157 | 53.127 | 1.00 | 23.20 | DIC |
| ATOM | 461 | C | ASP | 63 | 33.712 | 31.708 | 57.530 | 1.00 | 18.75 | DIC |
| ATOM | 462 | O | ASP | 63 | 34.127 | 31.004 | 58.448 | 1.00 | 18.31 | DIC |
| ATOM | 463 | N | VAL | 64 | 32.570 | 32.377 | 57.595 | 1.00 | 18.18 | DIC |
| ATOM | 464 | CA | VAL | 64 | 31.681 | 32.258 | 58.739 | 1.00 | 17.99 | DIC |
| ATOM | 465 | CB | VAL | 64 | 31.571 | 33.582 | 59.544 | 1.00 | 17.92 | DIC |
| ATOM | 466 | CG1 | VAL | 64 | 32.939 | 33.989 | 60.053 | 1.00 | 17.77 | DIC |
| ATOM | 467 | CG2 | VAL | 64 | 30.960 | 34.681 | 58.694 | 1.00 | 16.70 | DIC |
| ATOM | 468 | C | VAL | 64 | 30.325 | 31.889 | 58.161 | 1.00 | 17.88 | DIC |
| ATOM | 469 | O | VAL | 64 | 29.990 | 32.298 | 57.049 | 1.00 | 18.31 | DIC |
| ATOM | 470 | N | ASN | 65 | 29.562 | 31.085 | 58.891 | 1.00 | 17.42 | DIC |
| ATOM | 471 | CA | ASN | 65 | 28.241 | 30.685 | 58.430 | 1.00 | 17.62 | DIC |
| ATOM | 472 | CB | ASN | 65 | 27.924 | 29.261 | 58.885 | 1.00 | 18.50 | DIC |
| ATOM | 473 | CG | ASN | 65 | 26.616 | 28.743 | 58.306 | 1.00 | 20.54 | DIC |
| ATOM | 474 | OD1 | ASN | 65 | 25.714 | 29.521 | 57.968 | 1.00 | 20.86 | DIC |
| ATOM | 475 | ND2 | ASN | 65 | 26.499 | 27.420 | 58.200 | 1.00 | 19.60 | DIC |
| ATOM | 476 | C | ASN | 65 | 27.227 | 31.652 | 59.035 | 1.00 | 17.64 | DIC |
| ATOM | 477 | O | ASN | 65 | 26.932 | 31.585 | 60.230 | 1.00 | 16.65 | DIC |
| ATOM | 478 | N | PRO | 66 | 26.675 | 32.565 | 58.219 | 1.00 | 17.39 | DIC |
| ATOM | 479 | CD | PRO | 66 | 26.861 | 32.781 | 56.774 | 1.00 | 17.24 | DIC |
| ATOM | 480 | CA | PRO | 66 | 25.703 | 33.510 | 58.769 | 1.00 | 18.54 | DIC |
| ATOM | 481 | CB | PRO | 66 | 25.296 | 34.347 | 57.551 | 1.00 | 18.56 | DIC |
| ATOM | 482 | CG | PRO | 66 | 25.566 | 33.444 | 56.388 | 1.00 | 17.52 | DIC |
| ATOM | 483 | C | PRO | 66 | 24.516 | 32.866 | 59.480 | 1.00 | 19.19 | DIC |
| ATOM | 484 | O | PRO | 66 | 24.040 | 33.396 | 60.479 | 1.00 | 19.74 | DIC |
| ATOM | 485 | N | ALA | 67 | 24.058 | 31.717 | 58.984 | 1.00 | 19.24 | DIC |
| ATOM | 486 | CA | ALA | 67 | 22.913 | 31.029 | 59.583 | 1.00 | 18.98 | DIC |
| ATOM | 487 | CB | ALA | 67 | 22.603 | 29.740 | 58.805 | 1.00 | 19.76 | DIC |
| ATOM | 488 | C | ALA | 67 | 23.117 | 30.710 | 61.062 | 1.00 | 18.67 | DIC |
| ATOM | 489 | O | ALA | 67 | 22.154 | 30.496 | 61.797 | 1.00 | 18.27 | DIC |
| ATOM | 490 | N | ASP | 68 | 24.370 | 30.678 | 61.497 | 1.00 | 18.15 | DIC |
| ATOM | 491 | CA | ASP | 68 | 24.672 | 30.382 | 62.892 | 1.00 | 17.77 | DIC |
| ATOM | 492 | CB | ASP | 68 | 26.176 | 30.488 | 63.148 | 1.00 | 18.64 | DIC |
| ATOM | 493 | CG | ASP | 68 | 26.963 | 29.362 | 62.507 | 1.00 | 19.18 | DIC |
| ATOM | 494 | OD1 | ASP | 68 | 28.206 | 29.374 | 62.630 | 1.00 | 19.68 | DIC |
| ATOM | 495 | OD2 | ASP | 68 | 26.349 | 28.466 | 61.889 | 1.00 | 19.92 | DIC |
| ATOM | 496 | C | ASP | 68 | 23.948 | 31.307 | 63.868 | 1.00 | 17.23 | DIC |
| ATOM | 497 | O | ASP | 68 | 23.621 | 30.908 | 64.986 | 1.00 | 14.77 | DIC |
| ATOM | 498 | N | PHE | 69 | 23.705 | 32.544 | 63.452 | 1.00 | 17.46 | DIC |
| ATOM | 499 | CA | PHE | 69 | 23.061 | 33.513 | 64.332 | 1.00 | 18.56 | DIC |
| ATOM | 500 | CB | PHE | 69 | 23.895 | 34.801 | 64.374 | 1.00 | 19.00 | DIC |
| ATOM | 501 | CG | PHE | 69 | 25.382 | 34.558 | 64.338 | 1.00 | 18.44 | DIC |
| ATOM | 502 | CD1 | PHE | 69 | 26.069 | 34.550 | 63.126 | 1.00 | 18.34 | DIC |
| ATOM | 503 | CD2 | PHE | 69 | 26.080 | 34.274 | 65.506 | 1.00 | 17.81 | DIC |
| ATOM | 504 | CE1 | PHE | 69 | 27.435 | 34.256 | 63.081 | 1.00 | 17.68 | DIC |
| ATOM | 505 | CE2 | PHE | 69 | 27.441 | 33.980 | 65.477 | 1.00 | 17.08 | DIC |
| ATOM | 506 | CZ | PHE | 69 | 28.122 | 33.968 | 64.264 | 1.00 | 17.08 | DIC |
| ATOM | 507 | C | PHE | 69 | 21.642 | 33.825 | 63.889 | 1.00 | 19.64 | DIC |
| ATOM | 508 | O | PHE | 69 | 21.075 | 34.854 | 64.259 | 1.00 | 18.70 | DIC |
| ATOM | 509 | N | ALA | 70 | 21.067 | 32.919 | 63.109 | 1.00 | 20.08 | DIC |
| ATOM | 510 | CA | ALA | 70 | 19.718 | 33.094 | 62.593 | 1.00 | 21.26 | DIC |
| ATOM | 511 | CB | ALA | 70 | 19.302 | 31.846 | 61.818 | 1.00 | 22.16 | DIC |
| ATOM | 512 | C | ALA | 70 | 18.664 | 33.419 | 63.654 | 1.00 | 21.75 | DIC |
| ATOM | 513 | O | ALA | 70 | 17.783 | 34.246 | 63.421 | 1.00 | 22.84 | DIC |
| ATOM | 514 | N | GLY | 71 | 18.753 | 32.775 | 64.813 | 1.00 | 20.77 | DIC |
| ATOM | 515 | CA | GLY | 71 | 17.773 | 33.010 | 65.861 | 1.00 | 20.62 | DIC |
| ATOM | 516 | C | GLY | 71 | 17.817 | 34.344 | 66.599 | 1.00 | 20.26 | DIC |
| ATOM | 517 | O | GLY | 71 | 16.882 | 34.670 | 67.342 | 1.00 | 19.68 | DIC |
| ATOM | 518 | N | PHE | 72 | 18.878 | 35.120 | 66.392 | 1.00 | 18.49 | DIC |
| ATOM | 519 | CA | PHE | 72 | 19.042 | 36.412 | 67.063 | 1.00 | 17.73 | DIC |
| ATOM | 520 | CB | PHE | 72 | 20.536 | 36.763 | 67.145 | 1.00 | 18.72 | DIC |
| ATOM | 521 | CG | PHE | 72 | 21.348 | 35.802 | 67.980 | 1.00 | 20.16 | DIC |
| ATOM | 522 | CD1 | PHE | 72 | 22.724 | 35.960 | 68.097 | 1.00 | 20.37 | DIC |
| ATOM | 523 | CD2 | PHE | 72 | 20.737 | 34.760 | 68.668 | 1.00 | 20.44 | DIC |
| ATOM | 524 | CE1 | PHE | 72 | 23.477 | 35.100 | 68.888 | 1.00 | 21.46 | DIC |
| ATOM | 525 | CE2 | PHE | 72 | 21.484 | 33.892 | 69.462 | 1.00 | 21.52 | DIC |
| ATOM | 526 | CZ | PHE | 72 | 22.858 | 34.065 | 69.572 | 1.00 | 20.60 | DIC |
| ATOM | 527 | C | PHE | 72 | 18.274 | 37.551 | 66.386 | 1.00 | 16.90 | DIC |
| ATOM | 528 | O | PHE | 72 | 18.171 | 37.603 | 65.163 | 1.00 | 16.76 | DIC |
| ATOM | 529 | N | PRO | 73 | 17.738 | 38.492 | 67.176 | 1.00 | 16.37 | DIC |
| ATOM | 530 | CD | PRO | 73 | 17.908 | 38.675 | 68.626 | 1.00 | 16.89 | DIC |
| ATOM | 531 | CA | PRO | 73 | 16.984 | 39.609 | 66.600 | 1.00 | 16.59 | DIC |
| ATOM | 532 | CB | PRO | 73 | 16.616 | 40.447 | 67.825 | 1.00 | 16.80 | DIC |
| ATOM | 533 | CG | PRO | 73 | 17.705 | 40.164 | 68.773 | 1.00 | 17.34 | DIC |
| ATOM | 534 | C | PRO | 73 | 17.720 | 40.419 | 65.537 | 1.00 | 16.70 | DIC |
| ATOM | 535 | O | PRO | 73 | 18.924 | 40.645 | 65.629 | 1.00 | 15.30 | DIC |
| ATOM | 536 | N | LEU | 74 | 16.980 | 40.839 | 64.517 | 1.00 | 16.98 | DIC |
| ATOM | 537 | CA | LEU | 74 | 17.548 | 41.634 | 63.434 | 1.00 | 17.87 | DIC |
| ATOM | 538 | CB | LEU | 74 | 17.843 | 40.730 | 62.234 | 1.00 | 17.95 | DIC |
| ATOM | 539 | CG | LEU | 74 | 18.954 | 41.199 | 61.293 | 1.00 | 17.88 | DIC |
| ATOM | 540 | CD1 | LEU | 74 | 20.295 | 41.203 | 62.038 | 1.00 | 18.31 | DIC |
| ATOM | 541 | CD2 | LEU | 74 | 19.023 | 40.272 | 60.092 | 1.00 | 19.47 | DIC |
| ATOM | 542 | C | LEU | 74 | 16.520 | 42.713 | 63.068 | 1.00 | 18.09 | DIC |
| ATOM | 543 | O | LEU | 74 | 15.955 | 42.709 | 61.972 | 1.00 | 19.62 | DIC |
| ATOM | 544 | N | ASN | 75 | 16.290 | 43.630 | 64.005 | 1.00 | 17.72 | DIC |
| ATOM | 545 | CA | ASN | 75 | 15.322 | 44.715 | 63.846 | 1.00 | 17.11 | DIC |
| ATOM | 546 | CB | ASN | 75 | 14.735 | 45.110 | 65.229 | 1.00 | 17.62 | DIC |
| ATOM | 547 | CG | ASN | 75 | 15.817 | 45.329 | 66.338 | 1.00 | 20.20 | DIC |
| ATOM | 548 | OD1 | ASN | 75 | 16.525 | 44.396 | 66.744 | 1.00 | 19.66 | DIC |
| ATOM | 549 | ND2 | ASN | 75 | 15.911 | 46.566 | 66.844 | 1.00 | 20.31 | DIC |
| ATOM | 550 | C | ASN | 75 | 15.830 | 45.971 | 63.124 | 1.00 | 16.07 | DIC |
| ATOM | 551 | O | ASN | 75 | 15.053 | 46.683 | 62.477 | 1.00 | 15.53 | DIC |
| ATOM | 552 | N | ILE | 76 | 17.123 | 46.244 | 63.238 | 1.00 | 13.71 | DIC |
| ATOM | 553 | CA | ILE | 76 | 17.706 | 47.421 | 62.606 | 1.00 | 11.78 | DIC |
| ATOM | 554 | CB | ILE | 76 | 19.043 | 47.771 | 63.295 | 1.00 | 10.80 | DIC |
| ATOM | 555 | CG2 | ILE | 76 | 19.621 | 49.050 | 62.717 | 1.00 | 10.89 | DIC |
| ATOM | 556 | CG1 | ILE | 76 | 18.784 | 47.965 | 64.795 | 1.00 | 11.97 | DIC |
| ATOM | 557 | CD | ILE | 76 | 20.028 | 48.230 | 65.615 | 1.00 | 11.60 | DIC |
| ATOM | 558 | C | ILE | 76 | 17.880 | 47.165 | 61.112 | 1.00 | 10.59 | DIC |
| ATOM | 559 | O | ILE | 76 | 18.735 | 46.393 | 60.692 | 1.00 | 9.86 | DIC |
| ATOM | 560 | N | ARG | 77 | 17.047 | 47.823 | 60.312 | 1.00 | 10.58 | DIC |
| ATOM | 561 | CA | ARG | 77 | 17.046 | 47.629 | 58.865 | 1.00 | 9.71 | DIC |
| ATOM | 562 | CB | ARG | 77 | 16.017 | 48.560 | 58.224 | 1.00 | 10.51 | DIC |
| ATOM | 563 | CG | ARG | 77 | 15.694 | 48.227 | 56.770 | 1.00 | 12.15 | DIC |
| ATOM | 564 | CD | ARG | 77 | 14.702 | 49.229 | 56.194 | 1.00 | 14.05 | DIC |
| ATOM | 565 | NE | ARG | 77 | 14.405 | 48.956 | 54.792 | 1.00 | 15.41 | DIC |
| ATOM | 566 | CZ | ARG | 77 | 14.025 | 49.879 | 53.915 | 1.00 | 15.57 | DIC |
| ATOM | 567 | NH1 | ARG | 77 | 13.894 | 51.145 | 54.291 | 1.00 | 16.47 | DIC |
| ATOM | 568 | NH2 | ARG | 77 | 13.783 | 49.537 | 52.660 | 1.00 | 16.39 | DIC |
| ATOM | 569 | C | ARG | 77 | 18.370 | 47.769 | 58.125 | 1.00 | 9.25 | DIC |
| ATOM | 570 | O | ARG | 77 | 18.662 | 46.979 | 57.228 | 1.00 | 8.24 | DIC |
| ATOM | 571 | N | HIS | 78 | 19.175 | 48.765 | 58.475 | 1.00 | 9.54 | DIC |
| ATOM | 572 | CA | HIS | 78 | 20.435 | 48.953 | 57.763 | 1.00 | 9.73 | DIC |
| ATOM | 573 | CB | HIS | 78 | 20.945 | 50.392 | 57.948 | 1.00 | 9.99 | DIC |
| ATOM | 574 | CG | HIS | 78 | 21.495 | 50.677 | 59.313 | 1.00 | 9.83 | DIC |
| ATOM | 575 | CD2 | HIS | 78 | 20.910 | 51.193 | 60.417 | 1.00 | 9.26 | DIC |
| ATOM | 576 | ND1 | HIS | 78 | 22.801 | 50.399 | 59.664 | 1.00 | 12.11 | DIC |
| ATOM | 577 | CE1 | HIS | 78 | 22.992 | 50.731 | 60.927 | 1.00 | 10.27 | DIC |
| ATOM | 578 | NE2 | HIS | 78 | 21.860 | 51.213 | 61.407 | 1.00 | 10.71 | DIC |
| ATOM | 579 | C | HIS | 78 | 21.518 | 47.956 | 58.160 | 1.00 | 9.77 | DIC |
| ATOM | 580 | O | HIS | 78 | 22.634 | 48.014 | 57.643 | 1.00 | 9.16 | DIC |
| ATOM | 581 | N | ILE | 79 | 21.186 | 47.018 | 59.046 | 1.00 | 8.59 | DIC |
| ATOM | 582 | CA | ILE | 79 | 22.176 | 46.041 | 59.499 | 1.00 | 9.47 | DIC |
| ATOM | 583 | CB | ILE | 79 | 22.352 | 46.115 | 61.036 | 1.00 | 8.92 | DIC |
| ATOM | 584 | CG2 | ILE | 79 | 23.362 | 45.071 | 61.497 | 1.00 | 9.10 | DIC |
| ATOM | 585 | CG1 | ILE | 79 | 22.817 | 47.519 | 61.444 | 1.00 | 8.56 | DIC |
| ATOM | 586 | CD | ILE | 79 | 22.873 | 47.729 | 62.947 | 1.00 | 7.54 | DIC |
| ATOM | 587 | C | ILE | 79 | 21.855 | 44.597 | 59.129 | 1.00 | 9.95 | DIC |
| ATOM | 588 | O | ILE | 79 | 20.745 | 44.118 | 59.370 | 1.00 | 9.89 | DIC |
| ATOM | 589 | N | SER | 80 | 22.834 | 43.897 | 58.558 | 1.00 | 10.77 | DIC |
| ATOM | 590 | CA | SER | 80 | 22.644 | 42.489 | 58.185 | 1.00 | 10.59 | DIC |
| ATOM | 591 | CB | SER | 80 | 23.405 | 42.148 | 56.896 | 1.00 | 11.09 | DIC |
| ATOM | 592 | OG | SER | 80 | 24.799 | 42.152 | 57.115 | 1.00 | 11.31 | DIC |
| ATOM | 593 | C | SER | 80 | 23.102 | 41.551 | 59.304 | 1.00 | 10.27 | DIC |
| ATOM | 594 | O | SER | 80 | 23.784 | 41.967 | 60.250 | 1.00 | 9.68 | DIC |
| ATOM | 595 | N | ILE | 81 | 22.719 | 40.282 | 59.181 | 1.00 | 9.94 | DIC |
| ATOM | 596 | CA | ILE | 81 | 23.035 | 39.260 | 60.167 | 1.00 | 10.92 | DIC |
| ATOM | 597 | CB | ILE | 81 | 22.450 | 37.880 | 59.731 | 1.00 | 10.93 | DIC |
| ATOM | 598 | CG2 | ILE | 81 | 23.161 | 37.384 | 58.480 | 1.00 | 10.14 | DIC |
| ATOM | 599 | CG1 | ILE | 81 | 22.602 | 36.851 | 60.856 | 1.00 | 11.94 | DIC |
| ATOM | 600 | CD | ILE | 81 | 21.957 | 37.263 | 62.171 | 1.00 | 14.84 | DIC |
| ATOM | 601 | C | ILE | 81 | 24.530 | 39.131 | 60.452 | 1.00 | 10.21 | DIC |
| ATOM | 602 | O | ILE | 81 | 24.914 | 38.697 | 61.527 | 1.00 | 10.54 | DIC |
| ATOM | 603 | N | THR | 82 | 25.370 | 39.530 | 59.504 | 1.00 | 10.02 | DIC |
| ATOM | 604 | CA | THR | 82 | 26.814 | 39.440 | 59.690 | 1.00 | 11.39 | DIC |
| ATOM | 605 | CB | THR | 82 | 27.562 | 39.940 | 58.441 | 1.00 | 11.65 | DIC |
| ATOM | 606 | OG1 | THR | 82 | 27.204 | 39.125 | 57.325 | 1.00 | 14.61 | DIC |
| ATOM | 607 | CG2 | THR | 82 | 29.065 | 39.859 | 58.646 | 1.00 | 12.44 | DIC |
| ATOM | 608 | C | THR | 82 | 27.294 | 40.228 | 60.909 | 1.00 | 10.13 | DIC |
| ATOM | 609 | O | THR | 82 | 28.376 | 39.972 | 61.438 | 1.00 | 11.18 | DIC |
| ATOM | 610 | N | THR | 83 | 26.490 | 41.184 | 61.352 | 1.00 | 9.24 | DIC |
| ATOM | 611 | CA | THR | 83 | 26.839 | 41.991 | 62.518 | 1.00 | 8.73 | DIC |
| ATOM | 612 | CB | THR | 83 | 25.721 | 43.017 | 62.837 | 1.00 | 8.11 | DIC |
| ATOM | 613 | OG1 | THR | 83 | 26.189 | 43.930 | 63.833 | 1.00 | 6.94 | DIC |
| ATOM | 614 | CG2 | THR | 83 | 24.463 | 42.316 | 63.354 | 1.00 | 5.65 | DIC |
| ATOM | 615 | C | THR | 83 | 27.095 | 41.143 | 63.774 | 1.00 | 8.48 | DIC |
| ATOM | 616 | O | THR | 83 | 27.764 | 41.597 | 64.714 | 1.00 | 7.40 | DIC |
| ATOM | 617 | N | TYR | 84 | 26.571 | 39.917 | 63.796 | 1.00 | 7.85 | DIC |
| ATOM | 618 | CA | TYR | 84 | 26.750 | 39.036 | 64.961 | 1.00 | 7.48 | DIC |
| ATOM | 619 | CB | TYR | 84 | 25.541 | 38.118 | 65.149 | 1.00 | 8.12 | DIC |
| ATOM | 620 | CG | TYR | 84 | 24.309 | 38.765 | 65.730 | 1.00 | 8.79 | DIC |
| ATOM | 621 | CD1 | TYR | 84 | 23.223 | 39.089 | 64.920 | 1.00 | 8.55 | DIC |
| ATOM | 622 | CE1 | TYR | 84 | 22.061 | 39.639 | 65.456 | 1.00 | 8.25 | DIC |
| ATOM | 623 | CD2 | TYR | 84 | 24.208 | 39.014 | 67.100 | 1.00 | 9.35 | DIC |
| ATOM | 624 | CE2 | TYR | 84 | 23.047 | 39.569 | 67.647 | 1.00 | 8.56 | DIC |
| ATOM | 625 | CZ | TYR | 84 | 21.977 | 39.873 | 66.813 | 1.00 | 8.60 | DIC |
| ATOM | 626 | OH | TYR | 84 | 20.811 | 40.386 | 67.340 | 1.00 | 10.62 | DIC |
| ATOM | 627 | C | TYR | 84 | 27.988 | 38.142 | 64.917 | 1.00 | 7.79 | DIC |
| ATOM | 628 | O | TYR | 84 | 28.356 | 37.544 | 65.934 | 1.00 | 7.52 | DIC |
| ATOM | 629 | N | ALA | 85 | 28.631 | 38.046 | 63.756 | 1.00 | 7.28 | DIC |
| ATOM | 630 | CA | ALA | 85 | 29.802 | 37.172 | 63.608 | 1.00 | 8.87 | DIC |
| ATOM | 631 | CB | ALA | 85 | 30.381 | 37.297 | 62.200 | 1.00 | 8.53 | DIC |
| ATOM | 632 | C | ALA | 85 | 30.905 | 37.410 | 64.627 | 1.00 | 9.13 | DIC |
| ATOM | 633 | O | ALA | 85 | 31.639 | 36.483 | 64.993 | 1.00 | 8.63 | DIC |
| ATOM | 634 | N | ARG | 86 | 31.024 | 38.649 | 65.082 | 1.00 | 9.00 | DIC |
| ATOM | 635 | CA | ARG | 86 | 32.065 | 38.996 | 66.026 | 1.00 | 9.80 | DIC |
| ATOM | 636 | CB | ARG | 86 | 32.022 | 40.502 | 66.310 | 1.00 | 9.41 | DIC |
| ATOM | 637 | CG | ARG | 86 | 30.870 | 40.984 | 67.172 | 1.00 | 9.46 | DIC |
| ATOM | 638 | CD | ARG | 86 | 30.719 | 42.501 | 67.076 | 1.00 | 8.59 | DIC |
| ATOM | 639 | NE | ARG | 86 | 29.920 | 42.881 | 65.919 | 1.00 | 7.42 | DIC |
| ATOM | 640 | CZ | ARG | 86 | 29.754 | 44.128 | 65.491 | 1.00 | 8.59 | DIC |
| ATOM | 641 | NH1 | ARG | 86 | 30.342 | 45.144 | 66.115 | 1.00 | 7.18 | DIC |
| ATOM | 642 | NH2 | ARG | 86 | 28.974 | 44.361 | 64.449 | 1.00 | 9.43 | DIC |
| ATOM | 643 | C | ARG | 86 | 31.979 | 38.185 | 67.313 | 1.00 | 9.79 | DIC |
| ATOM | 644 | O | ARG | 86 | 32.978 | 38.004 | 68.000 | 1.00 | 10.73 | DIC |
| ATOM | 645 | N | LEU | 87 | 30.788 | 37.680 | 67.624 | 1.00 | 10.39 | DIC |
| ATOM | 646 | CA | LEU | 87 | 30.584 | 36.880 | 68.836 | 1.00 | 10.81 | DIC |
| ATOM | 647 | CB | LEU | 87 | 29.092 | 36.574 | 69.016 | 1.00 | 10.20 | DIC |
| ATOM | 648 | CG | LEU | 87 | 28.168 | 37.787 | 69.233 | 1.00 | 10.48 | DIC |
| ATOM | 649 | CD1 | LEU | 87 | 26.731 | 37.315 | 69.344 | 1.00 | 9.48 | DIC |
| ATOM | 650 | CD2 | LEU | 87 | 28.579 | 38.549 | 70.491 | 1.00 | 9.82 | DIC |
| ATOM | 651 | C | LEU | 87 | 31.394 | 35.574 | 68.884 | 1.00 | 10.74 | DIC |
| ATOM | 652 | O | LEU | 87 | 31.635 | 35.036 | 69.957 | 1.00 | 9.80 | DIC |
| ATOM | 653 | N | LYS | 88 | 31.811 | 35.067 | 67.725 | 1.00 | 11.78 | DIC |
| ATOM | 654 | CA | LYS | 88 | 32.599 | 33.831 | 67.665 | 1.00 | 11.01 | DIC |
| ATOM | 655 | CB | LYS | 88 | 32.009 | 32.882 | 66.612 | 1.00 | 11.35 | DIC |
| ATOM | 656 | CG | LYS | 88 | 30.771 | 32.128 | 67.071 | 1.00 | 9.90 | DIC |
| ATOM | 657 | CD | LYS | 88 | 30.341 | 31.114 | 66.016 | 1.00 | 11.02 | DIC |
| ATOM | 658 | CE | LYS | 88 | 29.384 | 30.086 | 66.593 | 1.00 | 10.58 | DIC |
| ATOM | 659 | NZ | LYS | 88 | 29.046 | 29.040 | 65.576 | 1.00 | 11.61 | DIC |
| ATOM | 660 | C | LYS | 88 | 34.071 | 34.097 | 67.332 | 1.00 | 11.82 | DIC |
| ATOM | 661 | O | LYS | 88 | 34.782 | 33.215 | 66.852 | 1.00 | 12.29 | DIC |
| ATOM | 662 | N | LEU | 89 | 34.531 | 35.307 | 67.611 | 1.00 | 12.36 | DIC |
| ATOM | 663 | CA | LEU | 89 | 35.902 | 35.686 | 67.303 | 1.00 | 13.57 | DIC |
| ATOM | 664 | CB | LEU | 89 | 36.144 | 37.121 | 67.772 | 1.00 | 15.15 | DIC |
| ATOM | 665 | CG | LEU | 89 | 36.980 | 38.051 | 66.888 | 1.00 | 16.39 | DIC |
| ATOM | 666 | CD1 | LEU | 89 | 36.680 | 37.846 | 65.418 | 1.00 | 16.80 | DIC |
| ATOM | 667 | CD2 | LEU | 89 | 36.684 | 39.481 | 67.294 | 1.00 | 17.50 | DIC |
| ATOM | 668 | C | LEU | 89 | 36.919 | 34.725 | 67.918 | 1.00 | 13.79 | DIC |
| ATOM | 669 | O | LEU | 89 | 37.951 | 34.420 | 67.310 | 1.00 | 12.41 | DIC |
| ATOM | 670 | N | GLY | 90 | 36.615 | 34.241 | 69.116 | 1.00 | 12.82 | DIC |
| ATOM | 671 | CA | GLY | 90 | 37.496 | 33.303 | 69.785 | 1.00 | 13.31 | DIC |
| ATOM | 672 | C | GLY | 90 | 37.608 | 31.968 | 69.057 | 1.00 | 13.77 | DIC |
| ATOM | 673 | O | GLY | 90 | 38.568 | 31.225 | 69.270 | 1.00 | 12.09 | DIC |
| ATOM | 674 | N | GLU | 91 | 36.625 | 31.662 | 68.210 | 1.00 | 13.93 | DIC |
| ATOM | 675 | CA | GLU | 91 | 36.608 | 30.423 | 67.431 | 1.00 | 14.80 | DIC |
| ATOM | 676 | CB | GLU | 91 | 35.172 | 29.930 | 67.221 | 1.00 | 15.97 | DIC |
| ATOM | 677 | CG | GLU | 91 | 34.422 | 29.493 | 68.475 | 1.00 | 18.54 | DIC |
| ATOM | 678 | CD | GLU | 91 | 33.035 | 28.944 | 68.145 | 1.00 | 20.57 | DIC |
| ATOM | 679 | OE1 | GLU | 91 | 32.929 | 28.123 | 67.211 | 1.00 | 21.70 | DIC |
| ATOM | 680 | OE2 | GLU | 91 | 32.057 | 29.326 | 68.816 | 1.00 | 20.80 | DIC |
| ATOM | 681 | C | GLU | 91 | 37.232 | 30.632 | 66.050 | 1.00 | 14.18 | DIC |
| ATOM | 682 | O | GLU | 91 | 37.833 | 29.717 | 65.478 | 1.00 | 13.91 | DIC |
| ATOM | 683 | N | TYR | 92 | 37.076 | 31.838 | 65.518 | 1.00 | 14.10 | DIC |
| ATOM | 684 | CA | TYR | 92 | 37.591 | 32.171 | 64.193 | 1.00 | 14.43 | DIC |
| ATOM | 685 | CB | TYR | 92 | 36.791 | 33.330 | 63.595 | 1.00 | 13.92 | DIC |
| ATOM | 686 | CG | TYR | 92 | 35.306 | 33.081 | 63.464 | 1.00 | 14.30 | DIC |
| ATOM | 687 | CD1 | TYR | 92 | 34.816 | 31.844 | 63.032 | 1.00 | 14.28 | DIC |
| ATOM | 688 | CE1 | TYR | 92 | 33.448 | 31.630 | 62.854 | 1.00 | 14.75 | DIC |
| ATOM | 689 | CD2 | TYR | 92 | 34.392 | 34.099 | 63.717 | 1.00 | 13.77 | DIC |
| ATOM | 690 | CE2 | TYR | 92 | 33.027 | 33.900 | 63.539 | 1.00 | 15.62 | DIC |
| ATOM | 691 | CZ | TYR | 92 | 32.560 | 32.663 | 63.105 | 1.00 | 14.94 | DIC |
| ATOM | 692 | OH | TYR | 92 | 31.213 | 32.477 | 62.906 | 1.00 | 15.54 | DIC |
| ATOM | 693 | C | TYR | 92 | 39.073 | 32.531 | 64.108 | 1.00 | 14.98 | DIC |
| ATOM | 694 | O | TYR | 92 | 39.701 | 32.328 | 63.072 | 1.00 | 14.45 | DIC |
| ATOM | 695 | N | ILE | 93 | 39.629 | 33.078 | 65.181 | 1.00 | 16.91 | DIC |
| ATOM | 696 | CA | ILE | 93 | 41.028 | 33.486 | 65.177 | 1.00 | 17.72 | DIC |
| ATOM | 697 | CB | ILE | 93 | 41.133 | 35.016 | 65.393 | 1.00 | 18.50 | DIC |
| ATOM | 698 | CG2 | ILE | 93 | 42.591 | 35.432 | 65.555 | 1.00 | 17.36 | DIC |
| ATOM | 699 | CG1 | ILE | 93 | 40.487 | 35.734 | 64.201 | 1.00 | 18.54 | DIC |
| ATOM | 700 | CD | ILE | 93 | 40.412 | 37.226 | 64.340 | 1.00 | 19.24 | DIC |
| ATOM | 701 | C | ILE | 93 | 41.858 | 32.751 | 66.220 | 1.00 | 18.60 | DIC |
| ATOM | 702 | O | ILE | 93 | 41.575 | 32.824 | 67.419 | 1.00 | 19.10 | DIC |
| ATOM | 703 | N | ALA | 94 | 42.885 | 32.043 | 65.758 | 1.00 | 18.48 | DIC |
| ATOM | 704 | CA | ALA | 94 | 43.748 | 31.279 | 66.651 | 1.00 | 20.07 | DIC |
| ATOM | 705 | CB | ALA | 94 | 43.905 | 29.870 | 66.122 | 1.00 | 19.66 | DIC |
| ATOM | 706 | C | ALA | 94 | 45.123 | 31.896 | 66.856 | 1.00 | 20.84 | DIC |
| ATOM | 707 | O | ALA | 94 | 45.715 | 31.768 | 67.931 | 1.00 | 20.49 | DIC |
| ATOM | 708 | N | ASP | 95 | 45.625 | 32.568 | 65.825 | 1.00 | 22.76 | DIC |
| ATOM | 709 | CA | ASP | 95 | 46.953 | 33.177 | 65.862 | 1.00 | 25.64 | DIC |
| ATOM | 710 | CB | ASP | 95 | 47.476 | 33.365 | 64.430 | 1.00 | 27.52 | DIC |
| ATOM | 711 | CG | ASP | 95 | 46.420 | 33.918 | 63.484 | 1.00 | 29.58 | DIC |
| ATOM | 712 | OD1 | ASP | 95 | 46.777 | 34.365 | 62.374 | 1.00 | 31.56 | DIC |
| ATOM | 713 | OD2 | ASP | 95 | 45.226 | 33.898 | 63.838 | 1.00 | 31.61 | DIC |
| ATOM | 714 | C | ASP | 95 | 47.129 | 34.494 | 66.625 | 1.00 | 25.59 | DIC |
| ATOM | 715 | O | ASP | 95 | 48.215 | 35.072 | 66.591 | 1.00 | 27.43 | DIC |
| ATOM | 716 | N | CYS | 96 | 46.102 | 34.967 | 67.325 | 1.00 | 24.71 | DIC |
| ATOM | 717 | CA | CYS | 96 | 46.232 | 36.235 | 68.043 | 1.00 | 23.67 | DIC |
| ATOM | 718 | CB | CYS | 96 | 45.513 | 37.347 | 67.274 | 1.00 | 23.87 | DIC |
| ATOM | 719 | SG | CYS | 96 | 46.038 | 37.496 | 65.578 | 1.00 | 26.37 | DIC |
| ATOM | 720 | C | CYS | 96 | 45.728 | 36.252 | 69.478 | 1.00 | 22.46 | DIC |
| ATOM | 721 | O | CYS | 96 | 44.646 | 35.741 | 69.776 | 1.00 | 22.34 | DIC |
| ATOM | 722 | N | ASP | 97 | 46.515 | 36.858 | 70.364 | 1.00 | 20.39 | DIC |
| ATOM | 723 | CA | ASP | 97 | 46.124 | 36.987 | 71.762 | 1.00 | 20.32 | DIC |
| ATOM | 724 | CB | ASP | 97 | 47.354 | 37.031 | 72.667 | 1.00 | 20.67 | DIC |
| ATOM | 725 | CG | ASP | 97 | 47.976 | 35.667 | 72.871 | 1.00 | 21.98 | DIC |
| ATOM | 726 | OD1 | ASP | 97 | 49.035 | 35.593 | 73.525 | 1.00 | 23.35 | DIC |
| ATOM | 727 | OD2 | ASP | 97 | 47.405 | 34.666 | 72.386 | 1.00 | 23.09 | DIC |
| ATOM | 728 | C | ASP | 97 | 45.323 | 38.279 | 71.925 | 1.00 | 18.84 | DIC |
| ATOM | 729 | O | ASP | 97 | 44.574 | 38.438 | 72.887 | 1.00 | 19.35 | DIC |
| ATOM | 730 | N | LYS | 98 | 45.503 | 39.198 | 70.978 | 1.00 | 16.98 | DIC |
| ATOM | 731 | CA | LYS | 98 | 44.814 | 40.488 | 70.975 | 1.00 | 15.16 | DIC |
| ATOM | 732 | CB | LYS | 98 | 45.704 | 41.580 | 71.585 | 1.00 | 15.88 | DIC |
| ATOM | 733 | CG | LYS | 98 | 45.142 | 42.998 | 71.424 | 1.00 | 16.96 | DIC |
| ATOM | 734 | CD | LYS | 98 | 46.102 | 44.099 | 71.908 | 1.00 | 16.73 | DIC |
| ATOM | 735 | CE | LYS | 98 | 46.335 | 44.046 | 73.413 | 1.00 | 16.78 | DIC |
| ATOM | 736 | NZ | LYS | 98 | 47.070 | 45.241 | 73.933 | 1.00 | 13.95 | DIC |
| ATOM | 737 | C | LYS | 98 | 44.479 | 40.890 | 69.540 | 1.00 | 14.03 | DIC |
| ATOM | 738 | O | LYS | 98 | 45.287 | 40.695 | 68.637 | 1.00 | 13.30 | DIC |
| ATOM | 739 | N | VAL | 99 | 43.281 | 41.431 | 69.332 | 1.00 | 11.95 | DIC |
| ATOM | 740 | CA | VAL | 99 | 42.868 | 41.900 | 68.012 | 1.00 | 11.37 | DIC |
| ATOM | 741 | CB | VAL | 99 | 42.024 | 40.852 | 67.236 | 1.00 | 11.54 | DIC |
| ATOM | 742 | CG1 | VAL | 99 | 42.877 | 39.639 | 66.882 | 1.00 | 11.02 | DIC |
| ATOM | 743 | CG2 | VAL | 99 | 40.794 | 40.453 | 68.058 | 1.00 | 10.46 | DIC |
| ATOM | 744 | C | VAL | 99 | 42.021 | 43.156 | 68.171 | 1.00 | 11.18 | DIC |
| ATOM | 745 | O | VAL | 99 | 41.327 | 43.322 | 69.171 | 1.00 | 10.93 | DIC |
| ATOM | 746 | N | LEU | 100 | 42.095 | 44.044 | 67.187 | 1.00 | 10.87 | DIC |
| ATOM | 747 | CA | LEU | 100 | 41.314 | 45.271 | 67.212 | 1.00 | 10.79 | DIC |
| ATOM | 748 | CB | LEU | 100 | 42.197 | 46.484 | 66.880 | 1.00 | 10.94 | DIC |
| ATOM | 749 | CG | LEU | 100 | 41.529 | 47.869 | 66.831 | 1.00 | 12.58 | DIC |
| ATOM | 750 | CD1 | LEU | 100 | 40.633 | 48.068 | 68.043 | 1.00 | 11.52 | DIC |
| ATOM | 751 | CD2 | LEU | 100 | 42.601 | 48.949 | 66.788 | 1.00 | 13.16 | DIC |
| ATOM | 752 | C | LEU | 100 | 40.219 | 45.108 | 66.169 | 1.00 | 10.87 | DIC |
| ATOM | 753 | O | LEU | 100 | 40.499 | 45.034 | 64.964 | 1.00 | 10.41 | DIC |
| ATOM | 754 | N | TYR | 101 | 38.981 | 45.026 | 66.651 | 1.00 | 10.75 | DIC |
| ATOM | 755 | CA | TYR | 101 | 37.811 | 44.861 | 65.806 | 1.00 | 10.15 | DIC |
| ATOM | 756 | CB | TYR | 101 | 36.714 | 44.096 | 66.553 | 1.00 | 11.92 | DIC |
| ATOM | 757 | CG | TYR | 101 | 35.519 | 43.787 | 65.679 | 1.00 | 12.22 | DIC |
| ATOM | 758 | CD1 | TYR | 101 | 35.489 | 42.629 | 64.912 | 1.00 | 13.77 | DIC |
| ATOM | 759 | CE1 | TYR | 101 | 34.442 | 42.355 | 64.055 | 1.00 | 14.00 | DIC |
| ATOM | 760 | CD2 | TYR | 101 | 34.452 | 44.677 | 65.566 | 1.00 | 13.42 | DIC |
| ATOM | 761 | CE2 | TYR | 101 | 33.377 | 44.409 | 64.690 | 1.00 | 13.71 | DIC |
| ATOM | 762 | CZ | TYR | 101 | 33.390 | 43.240 | 63.944 | 1.00 | 14.55 | DIC |
| ATOM | 763 | OH | TYR | 101 | 32.356 | 42.920 | 63.085 | 1.00 | 16.39 | DIC |
| ATOM | 764 | C | TYR | 101 | 37.251 | 46.216 | 65.411 | 1.00 | 10.17 | DIC |
| ATOM | 765 | O | TYR | 101 | 37.062 | 47.088 | 66.267 | 1.00 | 8.43 | DIC |
| ATOM | 766 | N | LEU | 102 | 36.972 | 46.385 | 64.121 | 1.00 | 8.96 | DIC |
| ATOM | 767 | CA | LEU | 102 | 36.409 | 47.629 | 63.617 | 1.00 | 9.15 | DIC |
| ATOM | 768 | CB | LEU | 102 | 37.429 | 48.370 | 62.757 | 1.00 | 9.36 | DIC |
| ATOM | 769 | CG | LEU | 102 | 38.760 | 48.796 | 63.379 | 1.00 | 10.19 | DIC |
| ATOM | 770 | CD1 | LEU | 102 | 39.640 | 49.399 | 62.292 | 1.00 | 9.39 | DIC |
| ATOM | 771 | CD2 | LEU | 102 | 38.507 | 49.791 | 64.514 | 1.00 | 9.64 | DIC |
| ATOM | 772 | C | LEU | 102 | 35.182 | 47.341 | 62.757 | 1.00 | 9.25 | DIC |
| ATOM | 773 | O | LEU | 102 | 35.178 | 46.379 | 61.988 | 1.00 | 9.46 | DIC |
| ATOM | 774 | N | ASP | 103 | 34.141 | 48.160 | 62.902 | 1.00 | 7.67 | DIC |
| ATOM | 775 | CA | ASP | 103 | 32.942 | 48.022 | 62.074 | 1.00 | 6.79 | DIC |
| ATOM | 776 | CB | ASP | 103 | 31.854 | 49.000 | 62.505 | 1.00 | 6.14 | DIC |
| ATOM | 777 | CG | ASP | 103 | 30.853 | 48.392 | 63.452 | 1.00 | 6.30 | DIC |
| ATOM | 778 | OD1 | ASP | 103 | 29.900 | 49.112 | 63.821 | 1.00 | 6.96 | DIC |
| ATOM | 779 | OD2 | ASP | 103 | 31.013 | 47.212 | 63.831 | 1.00 | 6.76 | DIC |
| ATOM | 780 | C | ASP | 103 | 33.407 | 48.438 | 60.689 | 1.00 | 6.85 | DIC |
| ATOM | 781 | O | ASP | 103 | 34.500 | 48.972 | 60.536 | 1.00 | 6.31 | DIC |
| ATOM | 782 | N | ILE | 104 | 32.564 | 48.234 | 59.690 | 1.00 | 8.06 | DIC |
| ATOM | 783 | CA | ILE | 104 | 32.914 | 48.593 | 58.318 | 1.00 | 7.88 | DIC |
| ATOM | 784 | CB | ILE | 104 | 32.057 | 47.796 | 57.315 | 1.00 | 9.07 | DIC |
| ATOM | 785 | CG2 | ILE | 104 | 32.357 | 48.246 | 55.891 | 1.00 | 9.25 | DIC |
| ATOM | 786 | CG1 | ILE | 104 | 32.297 | 46.290 | 57.504 | 1.00 | 8.98 | DIC |
| ATOM | 787 | CD | ILE | 104 | 33.728 | 45.819 | 57.279 | 1.00 | 9.78 | DIC |
| ATOM | 788 | C | ILE | 104 | 32.697 | 50.077 | 58.058 | 1.00 | 8.07 | DIC |
| ATOM | 789 | O | ILE | 104 | 33.375 | 50.675 | 57.223 | 1.00 | 7.27 | DIC |
| ATOM | 790 | N | ASP | 105 | 31.747 | 50.663 | 58.778 | 1.00 | 7.66 | DIC |
| ATOM | 791 | CA | ASP | 105 | 31.412 | 52.075 | 58.630 | 1.00 | 8.00 | DIC |
| ATOM | 792 | CB | ASP | 105 | 29.941 | 52.304 | 59.001 | 1.00 | 6.68 | DIC |
| ATOM | 793 | CG | ASP | 105 | 29.618 | 51.884 | 60.426 | 1.00 | 5.32 | DIC |
| ATOM | 794 | OD1 | ASP | 105 | 30.399 | 51.116 | 61.034 | 1.00 | 5.29 | DIC |
| ATOM | 795 | OD2 | ASP | 105 | 28.555 | 52.303 | 60.942 | 1.00 | 5.88 | DIC |
| ATOM | 796 | C | ASP | 105 | 32.303 | 52.991 | 59.469 | 1.00 | 8.38 | DIC |
| ATOM | 797 | O | ASP | 105 | 31.811 | 53.904 | 60.140 | 1.00 | 8.29 | DIC |
| ATOM | 798 | N | VAL | 106 | 33.609 | 52.733 | 59.437 | 1.00 | 9.42 | DIC |
| ATOM | 799 | CA | VAL | 106 | 34.565 | 53.552 | 60.170 | 1.00 | 10.80 | DIC |
| ATOM | 800 | CB | VAL | 106 | 35.344 | 52.758 | 61.252 | 1.00 | 11.19 | DIC |
| ATOM | 801 | CG1 | VAL | 106 | 34.380 | 52.108 | 62.219 | 1.00 | 10.51 | DIC |
| ATOM | 802 | CG2 | VAL | 106 | 36.258 | 51.732 | 60.595 | 1.00 | 11.56 | DIC |
| ATOM | 803 | C | VAL | 106 | 35.585 | 54.115 | 59.201 | 1.00 | 11.38 | DIC |
| ATOM | 804 | O | VAL | 106 | 35.790 | 53.579 | 58.112 | 1.00 | 10.35 | DIC |
| ATOM | 805 | N | LEU | 107 | 36.227 | 55.194 | 59.623 | 1.00 | 12.57 | DIC |
| ATOM | 806 | CA | LEU | 107 | 37.249 | 55.860 | 58.833 | 1.00 | 13.47 | DIC |
| ATOM | 807 | CB | LEU | 107 | 36.648 | 57.092 | 58.159 | 1.00 | 13.56 | DIC |
| ATOM | 808 | CG | LEU | 107 | 36.761 | 57.246 | 56.639 | 1.00 | 15.06 | DIC |
| ATOM | 809 | CD1 | LEU | 107 | 36.504 | 55.925 | 55.932 | 1.00 | 14.78 | DIC |
| ATOM | 810 | CD2 | LEU | 107 | 35.761 | 58.311 | 56.183 | 1.00 | 15.25 | DIC |
| ATOM | 811 | C | LEU | 107 | 38.346 | 56.262 | 59.820 | 1.00 | 13.50 | DIC |
| ATOM | 812 | O | LEU | 107 | 38.173 | 57.193 | 60.602 | 1.00 | 13.59 | DIC |
| ATOM | 813 | N | VAL | 108 | 39.457 | 55.532 | 59.798 | 1.00 | 13.84 | DIC |
| ATOM | 814 | CA | VAL | 108 | 40.581 | 55.799 | 60.689 | 1.00 | 14.29 | DIC |
| ATOM | 815 | CB | VAL | 108 | 41.566 | 54.613 | 60.695 | 1.00 | 13.17 | DIC |
| ATOM | 816 | CG1 | VAL | 108 | 42.776 | 54.931 | 61.572 | 1.00 | 11.37 | DIC |
| ATOM | 817 | CG2 | VAL | 108 | 40.866 | 53.378 | 61.199 | 1.00 | 11.78 | DIC |
| ATOM | 818 | C | VAL | 108 | 41.303 | 57.050 | 60.223 | 1.00 | 15.14 | DIC |
| ATOM | 819 | O | VAL | 108 | 41.829 | 57.080 | 59.111 | 1.00 | 15.87 | DIC |
| ATOM | 820 | N | ARG | 109 | 41.336 | 58.077 | 61.072 | 1.00 | 16.17 | DIC |
| ATOM | 821 | CA | ARG | 109 | 41.975 | 59.341 | 60.711 | 1.00 | 16.20 | DIC |
| ATOM | 822 | CB | ARG | 109 | 41.024 | 60.505 | 60.978 | 1.00 | 18.37 | DIC |
| ATOM | 823 | CG | ARG | 109 | 39.558 | 60.117 | 61.037 | 1.00 | 21.36 | DIC |
| ATOM | 824 | CD | ARG | 109 | 38.713 | 61.006 | 60.157 | 1.00 | 23.86 | DIC |
| ATOM | 825 | NE | ARG | 109 | 39.024 | 62.420 | 60.326 | 1.00 | 26.81 | DIC |
| ATOM | 826 | CZ | ARG | 109 | 38.490 | 63.389 | 59.584 | 1.00 | 27.63 | DIC |
| ATOM | 827 | NH1 | ARG | 109 | 38.828 | 64.653 | 59.792 | 1.00 | 28.71 | DIC |
| ATOM | 828 | NH2 | ARG | 109 | 37.614 | 63.089 | 58.635 | 1.00 | 27.97 | DIC |
| ATOM | 829 | C | ARG | 109 | 43.284 | 59.610 | 61.444 | 1.00 | 15.84 | DIC |
| ATOM | 830 | O | ARG | 109 | 43.962 | 60.596 | 61.159 | 1.00 | 15.77 | DIC |
| ATOM | 831 | N | ASP | 110 | 43.623 | 58.758 | 62.401 | 1.00 | 14.29 | DIC |
| ATOM | 832 | CA | ASP | 110 | 44.854 | 58.930 | 63.156 | 1.00 | 14.80 | DIC |
| ATOM | 833 | CB | ASP | 110 | 44.643 | 59.920 | 64.309 | 1.00 | 15.65 | DIC |
| ATOM | 834 | CG | ASP | 110 | 45.953 | 60.434 | 64.894 | 1.00 | 17.51 | DIC |
| ATOM | 835 | OD1 | ASP | 110 | 46.918 | 60.646 | 64.127 | 1.00 | 18.17 | DIC |
| ATOM | 836 | OD2 | ASP | 110 | 46.019 | 60.651 | 66.121 | 1.00 | 17.67 | DIC |
| ATOM | 837 | C | ASP | 110 | 45.302 | 57.577 | 63.683 | 1.00 | 15.01 | DIC |
| ATOM | 838 | O | ASP | 110 | 44.514 | 56.635 | 63.761 | 1.00 | 13.14 | DIC |
| ATOM | 839 | N | ARG | 111 | 46.580 | 57.493 | 64.026 | 1.00 | 15.41 | DIC |
| ATOM | 840 | CA | ARG | 111 | 47.185 | 56.274 | 64.532 | 1.00 | 15.96 | DIC |
| ATOM | 841 | CB | ARG | 111 | 48.589 | 56.587 | 65.040 | 1.00 | 18.93 | DIC |
| ATOM | 842 | CG | ARG | 111 | 49.292 | 55.406 | 65.638 | 1.00 | 21.10 | DIC |
| ATOM | 843 | CD | ARG | 111 | 49.885 | 55.785 | 66.979 | 1.00 | 26.48 | DIC |
| ATOM | 844 | NE | ARG | 111 | 50.917 | 56.811 | 66.896 | 1.00 | 29.16 | DIC |
| ATOM | 845 | CZ | ARG | 111 | 51.589 | 57.267 | 67.946 | 1.00 | 29.73 | DIC |
| ATOM | 846 | NH1 | ARG | 111 | 51.332 | 56.778 | 69.155 | 1.00 | 30.77 | DIC |
| ATOM | 847 | NH2 | ARG | 111 | 52.514 | 58.211 | 67.789 | 1.00 | 30.14 | DIC |
| ATOM | 848 | C | ARG | 111 | 46.381 | 55.601 | 65.643 | 1.00 | 15.25 | DIC |
| ATOM | 849 | O | ARG | 111 | 45.870 | 56.266 | 66.543 | 1.00 | 13.85 | DIC |
| ATOM | 850 | N | LEU | 112 | 46.294 | 54.275 | 65.572 | 1.00 | 13.25 | DIC |
| ATOM | 851 | CA | LEU | 112 | 45.569 | 53.467 | 66.555 | 1.00 | 13.07 | DIC |
| ATOM | 852 | CB | LEU | 112 | 44.820 | 52.336 | 65.845 | 1.00 | 12.62 | DIC |
| ATOM | 853 | CG | LEU | 112 | 43.346 | 52.448 | 65.422 | 1.00 | 13.47 | DIC |
| ATOM | 854 | CD1 | LEU | 112 | 42.780 | 53.836 | 65.654 | 1.00 | 11.94 | DIC |
| ATOM | 855 | CD2 | LEU | 112 | 43.243 | 52.037 | 63.970 | 1.00 | 13.50 | DIC |
| ATOM | 856 | C | LEU | 112 | 46.508 | 52.850 | 67.593 | 1.00 | 12.50 | DIC |
| ATOM | 857 | O | LEU | 112 | 46.067 | 52.151 | 68.497 | 1.00 | 11.71 | DIC |
| ATOM | 858 | N | THR | 113 | 47.802 | 53.107 | 67.462 | 1.00 | 11.56 | DIC |
| ATOM | 859 | CA | THR | 113 | 48.783 | 52.539 | 68.380 | 1.00 | 12.01 | DIC |
| ATOM | 860 | CB | THR | 113 | 50.184 | 53.060 | 68.041 | 1.00 | 11.89 | DIC |
| ATOM | 861 | OG1 | THR | 113 | 50.408 | 52.885 | 66.634 | 1.00 | 11.98 | DIC |
| ATOM | 862 | CG2 | THR | 113 | 51.261 | 52.293 | 68.820 | 1.00 | 13.32 | DIC |
| ATOM | 863 | C | THR | 113 | 48.475 | 52.770 | 69.864 | 1.00 | 12.09 | DIC |
| ATOM | 864 | O | THR | 113 | 48.600 | 51.851 | 70.672 | 1.00 | 13.00 | DIC |
| ATOM | 865 | N | PRO | 114 | 48.071 | 53.997 | 70.246 | 1.00 | 11.73 | DIC |
| ATOM | 866 | CD | PRO | 114 | 48.006 | 55.257 | 69.488 | 1.00 | 11.33 | DIC |
| ATOM | 867 | CA | PRO | 114 | 47.764 | 54.227 | 71.662 | 1.00 | 12.05 | DIC |
| ATOM | 868 | CB | PRO | 114 | 47.313 | 55.685 | 71.685 | 1.00 | 11.93 | DIC |
| ATOM | 869 | CG | PRO | 114 | 48.143 | 56.295 | 70.585 | 1.00 | 12.09 | DIC |
| ATOM | 870 | C | PRO | 114 | 46.674 | 53.271 | 72.150 | 1.00 | 11.56 | DIC |
| ATOM | 871 | O | PRO | 114 | 46.743 | 52.757 | 73.266 | 1.00 | 11.37 | DIC |
| ATOM | 872 | N | LEU | 115 | 45.664 | 53.037 | 71.314 | 1.00 | 10.81 | DIC |
| ATOM | 873 | CA | LEU | 115 | 44.590 | 52.120 | 71.683 | 1.00 | 10.71 | DIC |
| ATOM | 874 | CB | LEU | 115 | 43.423 | 52.211 | 70.689 | 1.00 | 10.70 | DIC |
| ATOM | 875 | CG | LEU | 115 | 42.277 | 51.209 | 70.886 | 1.00 | 10.89 | DIC |
| ATOM | 876 | CD1 | LEU | 115 | 41.718 | 51.306 | 72.293 | 1.00 | 11.32 | DIC |
| ATOM | 877 | CD2 | LEU | 115 | 41.184 | 51.495 | 69.857 | 1.00 | 10.96 | DIC |
| ATOM | 878 | C | LEU | 115 | 45.150 | 50.703 | 71.693 | 1.00 | 9.83 | DIC |
| ATOM | 879 | O | LEU | 115 | 44.966 | 49.967 | 72.648 | 1.00 | 11.08 | DIC |
| ATOM | 880 | N | TRP | 116 | 45.850 | 50.338 | 70.628 | 1.00 | 9.48 | DIC |
| ATOM | 881 | CA | TRP | 116 | 46.436 | 49.007 | 70.511 | 1.00 | 10.20 | DIC |
| ATOM | 882 | CB | TRP | 116 | 47.167 | 48.873 | 69.164 | 1.00 | 9.44 | DIC |
| ATOM | 883 | CG | TRP | 116 | 47.899 | 47.551 | 68.983 | 1.00 | 10.50 | DIC |
| ATOM | 884 | CD2 | TRP | 116 | 47.327 | 46.288 | 68.606 | 1.00 | 10.45 | DIC |
| ATOM | 885 | CE2 | TRP | 116 | 48.374 | 45.337 | 68.603 | 1.00 | 11.26 | DIC |
| ATOM | 886 | CE3 | TRP | 116 | 46.033 | 45.869 | 68.276 | 1.00 | 11.37 | DIC |
| ATOM | 887 | CD1 | TRP | 116 | 49.228 | 47.320 | 69.185 | 1.00 | 10.48 | DIC |
| ATOM | 888 | NE1 | TRP | 116 | 49.522 | 45.992 | 68.956 | 1.00 | 11.48 | DIC |
| ATOM | 889 | CZ2 | TRP | 116 | 48.166 | 43.988 | 68.276 | 1.00 | 10.98 | DIC |
| ATOM | 890 | CZ3 | TRP | 116 | 45.827 | 44.529 | 67.951 | 1.00 | 10.53 | DIC |
| ATOM | 891 | CH2 | TRP | 116 | 46.892 | 43.605 | 67.954 | 1.00 | 9.77 | DIC |
| ATOM | 892 | C | TRP | 116 | 47.388 | 48.674 | 71.662 | 1.00 | 11.01 | DIC |
| ATOM | 893 | O | TRP | 116 | 47.439 | 47.533 | 72.117 | 1.00 | 11.69 | DIC |
| ATOM | 894 | N | ASP | 117 | 48.133 | 49.667 | 72.138 | 1.00 | 11.59 | DIC |
| ATOM | 895 | CA | ASP | 117 | 49.084 | 49.442 | 73.223 | 1.00 | 12.22 | DIC |
| ATOM | 896 | CB | ASP | 117 | 50.162 | 50.534 | 73.238 | 1.00 | 12.26 | DIC |
| ATOM | 897 | CG | ASP | 117 | 51.120 | 50.425 | 72.077 | 1.00 | 13.59 | DIC |
| ATOM | 898 | OD1 | ASP | 117 | 51.257 | 49.321 | 71.515 | 1.00 | 12.25 | DIC |
| ATOM | 899 | OD2 | ASP | 117 | 51.752 | 51.449 | 71.745 | 1.00 | 14.88 | DIC |
| ATOM | 900 | C | ASP | 117 | 48.438 | 49.390 | 74.599 | 1.00 | 13.46 | DIC |
| ATOM | 901 | O | ASP | 117 | 49.125 | 49.168 | 75.598 | 1.00 | 13.16 | DIC |
| ATOM | 902 | N | THR | 118 | 47.127 | 49.606 | 74.660 | 1.00 | 14.71 | DIC |
| ATOM | 903 | CA | THR | 118 | 46.425 | 49.575 | 75.939 | 1.00 | 16.37 | DIC |
| ATOM | 904 | CB | THR | 118 | 44.938 | 49.978 | 75.779 | 1.00 | 16.43 | DIC |
| ATOM | 905 | OG1 | THR | 118 | 44.862 | 51.305 | 75.244 | 1.00 | 16.77 | DIC |
| ATOM | 906 | CG2 | THR | 118 | 44.220 | 49.948 | 77.129 | 1.00 | 17.74 | DIC |
| ATOM | 907 | C | THR | 118 | 46.494 | 48.183 | 76.555 | 1.00 | 17.33 | DIC |
| ATOM | 908 | O | THR | 118 | 46.284 | 47.184 | 75.871 | 1.00 | 16.96 | DIC |
| ATOM | 909 | N | ASP | 119 | 46.792 | 48.123 | 77.849 | 1.00 | 18.74 | DIC |
| ATOM | 910 | CA | ASP | 119 | 46.871 | 46.843 | 78.552 | 1.00 | 20.28 | DIC |
| ATOM | 911 | CB | ASP | 119 | 47.930 | 46.919 | 79.656 | 1.00 | 22.23 | DIC |
| ATOM | 912 | CG | ASP | 119 | 47.948 | 45.685 | 80.536 | 1.00 | 24.82 | DIC |
| ATOM | 913 | OD1 | ASP | 119 | 47.718 | 44.570 | 80.020 | 1.00 | 25.22 | DIC |
| ATOM | 914 | OD2 | ASP | 119 | 48.211 | 45.832 | 81.750 | 1.00 | 27.28 | DIC |
| ATOM | 915 | C | ASP | 119 | 45.496 | 46.519 | 79.137 | 1.00 | 20.16 | DIC |
| ATOM | 916 | O | ASP | 119 | 45.038 | 47.173 | 80.076 | 1.00 | 19.67 | DIC |
| ATOM | 917 | N | LEU | 120 | 44.844 | 45.511 | 78.565 | 1.00 | 19.65 | DIC |
| ATOM | 918 | CA | LEU | 120 | 43.505 | 45.102 | 78.985 | 1.00 | 19.29 | DIC |
| ATOM | 919 | CB | LEU | 120 | 42.851 | 44.242 | 77.892 | 1.00 | 18.66 | DIC |
| ATOM | 920 | CG | LEU | 120 | 42.554 | 44.848 | 76.517 | 1.00 | 18.25 | DIC |
| ATOM | 921 | CD1 | LEU | 120 | 43.830 | 45.349 | 75.890 | 1.00 | 19.33 | DIC |
| ATOM | 922 | CD2 | LEU | 120 | 41.924 | 43.796 | 75.627 | 1.00 | 17.41 | DIC |
| ATOM | 923 | C | LEU | 120 | 43.457 | 44.333 | 80.303 | 1.00 | 19.39 | DIC |
| ATOM | 924 | O | LEU | 120 | 42.377 | 44.121 | 80.857 | 1.00 | 19.64 | DIC |
| ATOM | 925 | N | GLY | 121 | 44.611 | 43.908 | 80.804 | 1.00 | 19.06 | DIC |
| ATOM | 926 | CA | GLY | 121 | 44.618 | 43.151 | 82.044 | 1.00 | 19.45 | DIC |
| ATOM | 927 | C | GLY | 121 | 43.724 | 41.933 | 81.890 | 1.00 | 19.65 | DIC |
| ATOM | 928 | O | GLY | 121 | 43.805 | 41.225 | 80.884 | 1.00 | 19.83 | DIC |
| ATOM | 929 | N | ASN | 122 | 42.863 | 41.672 | 82.866 | 1.00 | 19.86 | DIC |
| ATOM | 930 | CA | ASN | 122 | 41.982 | 40.518 | 82.747 | 1.00 | 20.32 | DIC |
| ATOM | 931 | CB | ASN | 122 | 41.870 | 39.765 | 84.079 | 1.00 | 22.30 | DIC |
| ATOM | 932 | CG | ASN | 122 | 41.270 | 38.374 | 83.910 | 1.00 | 23.86 | DIC |
| ATOM | 933 | OD1 | ASN | 122 | 41.773 | 37.559 | 83.132 | 1.00 | 24.87 | DIC |
| ATOM | 934 | ND2 | ASN | 122 | 40.193 | 38.099 | 84.637 | 1.00 | 24.89 | DIC |
| ATOM | 935 | C | ASN | 122 | 40.597 | 40.944 | 82.271 | 1.00 | 19.00 | DIC |
| ATOM | 936 | O | ASN | 122 | 39.629 | 40.201 | 82.424 | 1.00 | 18.30 | DIC |
| ATOM | 937 | N | ASN | 123 | 40.507 | 42.149 | 81.707 | 1.00 | 17.37 | DIC |
| ATOM | 938 | CA | ASN | 123 | 39.238 | 42.651 | 81.186 | 1.00 | 16.29 | DIC |
| ATOM | 939 | CB | ASN | 123 | 39.313 | 44.147 | 80.858 | 1.00 | 15.93 | DIC |
| ATOM | 940 | CG | ASN | 123 | 39.347 | 45.014 | 82.090 | 1.00 | 16.95 | DIC |
| ATOM | 941 | OD1 | ASN | 123 | 40.344 | 45.684 | 82.366 | 1.00 | 17.08 | DIC |
| ATOM | 942 | ND2 | ASN | 123 | 38.257 | 45.013 | 82.839 | 1.00 | 16.13 | DIC |
| ATOM | 943 | C | ASN | 123 | 38.935 | 41.895 | 79.908 | 1.00 | 15.30 | DIC |
| ATOM | 944 | O | ASN | 123 | 39.842 | 41.387 | 79.253 | 1.00 | 15.35 | DIC |
| ATOM | 945 | N | TRP | 124 | 37.656 | 41.834 | 79.551 | 1.00 | 14.51 | DIC |
| ATOM | 946 | CA | TRP | 124 | 37.238 | 41.137 | 78.350 | 1.00 | 12.04 | DIC |
| ATOM | 947 | CB | TRP | 124 | 35.727 | 40.935 | 78.354 | 1.00 | 12.24 | DIC |
| ATOM | 948 | CG | TRP | 124 | 35.220 | 39.973 | 79.384 | 1.00 | 12.53 | DIC |
| ATOM | 949 | CD2 | TRP | 124 | 35.162 | 38.547 | 79.264 | 1.00 | 12.85 | DIC |
| ATOM | 950 | CE2 | TRP | 124 | 34.518 | 38.054 | 80.422 | 1.00 | 12.47 | DIC |
| ATOM | 951 | CE3 | TRP | 124 | 35.587 | 37.635 | 78.285 | 1.00 | 12.98 | DIC |
| ATOM | 952 | CD1 | TRP | 124 | 34.635 | 40.282 | 80.583 | 1.00 | 11.72 | DIC |
| ATOM | 953 | NE1 | TRP | 124 | 34.206 | 39.131 | 81.210 | 1.00 | 12.73 | DIC |
| ATOM | 954 | CZ2 | TRP | 124 | 34.285 | 36.689 | 80.627 | 1.00 | 13.82 | DIC |
| ATOM | 955 | CZ3 | TRP | 124 | 35.354 | 36.277 | 78.491 | 1.00 | 14.04 | DIC |
| ATOM | 956 | CH2 | TRP | 124 | 34.709 | 35.821 | 79.652 | 1.00 | 13.35 | DIC |
| ATOM | 957 | C | TRP | 124 | 37.628 | 41.904 | 77.094 | 1.00 | 11.46 | DIC |
| ATOM | 958 | O | TRP | 124 | 37.992 | 41.308 | 76.088 | 1.00 | 10.17 | DIC |
| ATOM | 959 | N | LEU | 125 | 37.558 | 43.227 | 77.158 | 1.00 | 10.45 | DIC |
| ATOM | 960 | CA | LEU | 125 | 37.898 | 44.035 | 76.000 | 1.00 | 11.28 | DIC |
| ATOM | 961 | CB | LEU | 125 | 36.841 | 43.844 | 74.903 | 1.00 | 11.69 | DIC |
| ATOM | 962 | CG | LEU | 125 | 35.439 | 44.448 | 75.080 | 1.00 | 12.44 | DIC |
| ATOM | 963 | CD1 | LEU | 125 | 34.499 | 43.821 | 74.070 | 1.00 | 12.81 | DIC |
| ATOM | 964 | CD2 | LEU | 125 | 34.910 | 44.193 | 76.465 | 1.00 | 13.63 | DIC |
| ATOM | 965 | C | LEU | 125 | 37.995 | 45.505 | 76.366 | 1.00 | 11.38 | DIC |
| ATOM | 966 | O | LEU | 125 | 37.713 | 45.897 | 77.500 | 1.00 | 11.51 | DIC |
| ATOM | 967 | N | GLY | 126 | 38.415 | 46.305 | 75.397 | 1.00 | 10.75 | DIC |
| ATOM | 968 | CA | GLY | 126 | 38.526 | 47.735 | 75.592 | 1.00 | 11.81 | DIC |
| ATOM | 969 | C | GLY | 126 | 37.590 | 48.359 | 74.577 | 1.00 | 11.37 | DIC |
| ATOM | 970 | O | GLY | 126 | 37.579 | 47.939 | 73.418 | 1.00 | 11.54 | DIC |
| ATOM | 971 | N | ALA | 127 | 36.804 | 49.345 | 74.998 | 1.00 | 10.41 | DIC |
| ATOM | 972 | CA | ALA | 127 | 35.860 | 50.006 | 74.095 | 1.00 | 8.96 | DIC |
| ATOM | 973 | CB | ALA | 127 | 34.577 | 49.190 | 74.008 | 1.00 | 8.65 | DIC |
| ATOM | 974 | C | ALA | 127 | 35.536 | 51.431 | 74.535 | 1.00 | 9.67 | DIC |
| ATOM | 975 | O | ALA | 127 | 35.735 | 51.795 | 75.701 | 1.00 | 7.21 | DIC |
| ATOM | 976 | N | SER | 128 | 35.039 | 52.240 | 73.599 | 1.00 | 9.58 | DIC |
| ATOM | 977 | CA | SER | 128 | 34.675 | 53.617 | 73.916 | 1.00 | 10.71 | DIC |
| ATOM | 978 | CB | SER | 128 | 34.895 | 54.538 | 72.711 | 1.00 | 10.39 | DIC |
| ATOM | 979 | OG | SER | 128 | 36.250 | 54.510 | 72.286 | 1.00 | 10.31 | DIC |
| ATOM | 980 | C | SER | 128 | 33.206 | 53.644 | 74.321 | 1.00 | 11.60 | DIC |
| ATOM | 981 | O | SER | 128 | 32.408 | 52.818 | 73.857 | 1.00 | 10.65 | DIC |
| ATOM | 982 | N | ILE | 129 | 32.859 | 54.597 | 75.179 | 1.00 | 11.17 | DIC |
| ATOM | 983 | CA | ILE | 129 | 31.496 | 54.744 | 75.675 | 1.00 | 11.89 | DIC |
| ATOM | 984 | CB | ILE | 129 | 31.484 | 55.693 | 76.909 | 1.00 | 12.55 | DIC |
| ATOM | 985 | CG2 | ILE | 129 | 30.059 | 55.997 | 77.348 | 1.00 | 11.82 | DIC |
| ATOM | 986 | CG1 | ILE | 129 | 32.269 | 55.040 | 78.049 | 1.00 | 13.27 | DIC |
| ATOM | 987 | CD | ILE | 129 | 32.567 | 55.968 | 79.199 | 1.00 | 14.30 | DIC |
| ATOM | 988 | C | ILE | 129 | 30.538 | 55.257 | 74.600 | 1.00 | 13.23 | DIC |
| ATOM | 989 | O | ILE | 129 | 30.913 | 56.068 | 73.746 | 1.00 | 13.13 | DIC |
| ATOM | 990 | N | ASP | 130 | 29.300 | 54.771 | 74.647 | 1.00 | 12.89 | DIC |
| ATOM | 991 | CA | ASP | 130 | 28.280 | 55.167 | 73.693 | 1.00 | 13.14 | DIC |
| ATOM | 992 | CB | ASP | 130 | 27.447 | 53.944 | 73.284 | 1.00 | 12.35 | DIC |
| ATOM | 993 | CG | ASP | 130 | 26.610 | 54.195 | 72.039 | 1.00 | 12.78 | DIC |
| ATOM | 994 | OD1 | ASP | 130 | 25.776 | 55.130 | 72.033 | 1.00 | 14.26 | DIC |
| ATOM | 995 | OD2 | ASP | 130 | 26.784 | 53.454 | 71.060 | 1.00 | 13.48 | DIC |
| ATOM | 996 | C | ASP | 130 | 27.379 | 56.215 | 74.340 | 1.00 | 13.29 | DIC |
| ATOM | 997 | O | ASP | 130 | 26.483 | 55.878 | 75.112 | 1.00 | 13.88 | DIC |
| ATOM | 998 | N | LEU | 131 | 27.626 | 57.483 | 74.027 | 1.00 | 14.55 | DIC |
| ATOM | 999 | CA | LEU | 131 | 26.843 | 58.597 | 74.574 | 1.00 | 14.53 | DIC |
| ATOM | 1000 | CB | LEU | 131 | 27.401 | 59.933 | 74.071 | 1.00 | 15.55 | DIC |
| ATOM | 1001 | CG | LEU | 131 | 28.630 | 60.539 | 74.752 | 1.00 | 16.42 | DIC |
| ATOM | 1002 | CD1 | LEU | 131 | 29.698 | 59.495 | 75.006 | 1.00 | 17.97 | DIC |
| ATOM | 1003 | CD2 | LEU | 131 | 29.161 | 61.654 | 73.872 | 1.00 | 16.87 | DIC |
| ATOM | 1004 | C | LEU | 131 | 25.364 | 58.516 | 74.215 | 1.00 | 14.67 | DIC |
| ATOM | 1005 | O | LEU | 131 | 24.496 | 58.874 | 75.016 | 1.00 | 14.54 | DIC |
| ATOM | 1006 | N | PHE | 132 | 25.083 | 58.059 | 73.002 | 1.00 | 13.83 | DIC |
| ATOM | 1007 | CA | PHE | 132 | 23.714 | 57.935 | 72.528 | 1.00 | 14.23 | DIC |
| ATOM | 1008 | CB | PHE | 132 | 23.722 | 57.462 | 71.070 | 1.00 | 14.97 | DIC |
| ATOM | 1009 | CG | PHE | 132 | 22.354 | 57.288 | 70.481 | 1.00 | 16.38 | DIC |
| ATOM | 1010 | CD1 | PHE | 132 | 21.728 | 56.044 | 70.494 | 1.00 | 17.39 | DIC |
| ATOM | 1011 | CD2 | PHE | 132 | 21.685 | 58.368 | 69.924 | 1.00 | 17.06 | DIC |
| ATOM | 1012 | CE1 | PHE | 132 | 20.456 | 55.877 | 69.961 | 1.00 | 17.85 | DIC |
| ATOM | 1013 | CE2 | PHE | 132 | 20.405 | 58.215 | 69.385 | 1.00 | 19.05 | DIC |
| ATOM | 1014 | CZ | PHE | 132 | 19.790 | 56.962 | 69.404 | 1.00 | 19.20 | DIC |
| ATOM | 1015 | C | PHE | 132 | 22.896 | 56.974 | 73.398 | 1.00 | 14.47 | DIC |
| ATOM | 1016 | O | PHE | 132 | 21.769 | 57.290 | 73.793 | 1.00 | 14.19 | DIC |
| ATOM | 1017 | N | VAL | 133 | 23.464 | 55.807 | 73.703 | 1.00 | 12.67 | DIC |
| ATOM | 1018 | CA | VAL | 133 | 22.757 | 54.829 | 74.523 | 1.00 | 13.81 | DIC |
| ATOM | 1019 | CB | VAL | 133 | 23.396 | 53.433 | 74.433 | 1.00 | 12.45 | DIC |
| ATOM | 1020 | CG1 | VAL | 133 | 22.658 | 52.469 | 75.359 | 1.00 | 13.71 | DIC |
| ATOM | 1021 | CG2 | VAL | 133 | 23.330 | 52.924 | 73.002 | 1.00 | 12.09 | DIC |
| ATOM | 1022 | C | VAL | 133 | 22.734 | 55.248 | 75.983 | 1.00 | 14.81 | DIC |
| ATOM | 1023 | O | VAL | 133 | 21.710 | 55.125 | 76.659 | 1.00 | 15.65 | DIC |
| ATOM | 1024 | N | GLU | 134 | 23.864 | 55.753 | 76.462 | 1.00 | 16.17 | DIC |
| ATOM | 1025 | CA | GLU | 134 | 23.987 | 56.184 | 77.845 | 1.00 | 17.03 | DIC |
| ATOM | 1026 | CB | GLU | 134 | 25.408 | 56.700 | 78.089 | 1.00 | 17.49 | DIC |
| ATOM | 1027 | CG | GLU | 134 | 25.763 | 56.880 | 79.553 | 1.00 | 20.47 | DIC |
| ATOM | 1028 | CD | GLU | 134 | 25.781 | 55.575 | 80.329 | 1.00 | 18.73 | DIC |
| ATOM | 1029 | OE1 | GLU | 134 | 25.626 | 55.635 | 81.556 | 1.00 | 20.44 | DIC |
| ATOM | 1030 | OE2 | GLU | 134 | 25.959 | 54.496 | 79.729 | 1.00 | 20.43 | DIC |
| ATOM | 1031 | C | GLU | 134 | 22.955 | 57.252 | 78.230 | 1.00 | 18.44 | DIC |
| ATOM | 1032 | O | GLU | 134 | 22.529 | 57.321 | 79.378 | 1.00 | 18.74 | DIC |
| ATOM | 1033 | N | ARG | 135 | 22.540 | 58.082 | 77.281 | 1.00 | 18.60 | DIC |
| ATOM | 1034 | CA | ARG | 135 | 21.560 | 59.115 | 77.594 | 1.00 | 20.63 | DIC |
| ATOM | 1035 | CB | ARG | 135 | 21.897 | 60.419 | 76.867 | 1.00 | 22.18 | DIC |
| ATOM | 1036 | CG | ARG | 135 | 21.810 | 60.353 | 75.362 | 1.00 | 25.33 | DIC |
| ATOM | 1037 | CD | ARG | 135 | 22.541 | 61.540 | 74.759 | 1.00 | 28.60 | DIC |
| ATOM | 1038 | NE | ARG | 135 | 21.996 | 62.820 | 75.204 | 1.00 | 30.75 | DIC |
| ATOM | 1039 | CZ | ARG | 135 | 20.972 | 63.444 | 74.626 | 1.00 | 31.82 | DIC |
| ATOM | 1040 | NH1 | ARG | 135 | 20.547 | 64.606 | 75.110 | 1.00 | 32.80 | DIC |
| ATOM | 1041 | NH2 | ARG | 135 | 20.379 | 62.917 | 73.560 | 1.00 | 32.31 | DIC |
| ATOM | 1042 | C | ARG | 135 | 20.140 | 58.682 | 77.261 | 1.00 | 20.30 | DIC |
| ATOM | 1043 | O | ARG | 135 | 19.215 | 59.488 | 77.293 | 1.00 | 20.01 | DIC |
| ATOM | 1044 | N | GLN | 136 | 19.975 | 57.408 | 76.928 | 1.00 | 20.15 | DIC |
| ATOM | 1045 | CA | GLN | 136 | 18.660 | 56.864 | 76.631 | 1.00 | 20.32 | DIC |
| ATOM | 1046 | CB | GLN | 136 | 18.789 | 55.671 | 75.688 | 1.00 | 20.65 | DIC |
| ATOM | 1047 | CG | GLN | 136 | 17.480 | 55.152 | 75.143 | 1.00 | 21.22 | DIC |
| ATOM | 1048 | CD | GLN | 136 | 17.681 | 53.987 | 74.193 | 1.00 | 22.55 | DIC |
| ATOM | 1049 | OE1 | GLN | 136 | 18.653 | 53.953 | 73.436 | 1.00 | 22.60 | DIC |
| ATOM | 1050 | NE2 | GLN | 136 | 16.757 | 53.031 | 74.218 | 1.00 | 22.18 | DIC |
| ATOM | 1051 | C | GLN | 136 | 18.143 | 56.411 | 77.993 | 1.00 | 21.14 | DIC |
| ATOM | 1052 | O | GLN | 136 | 18.358 | 55.271 | 78.397 | 1.00 | 19.68 | DIC |
| ATOM | 1053 | N | GLU | 137 | 17.480 | 57.326 | 78.698 | 1.00 | 22.64 | DIC |
| ATOM | 1054 | CA | GLU | 137 | 16.955 | 57.086 | 80.045 | 1.00 | 24.24 | DIC |
| ATOM | 1055 | CB | GLU | 137 | 15.856 | 58.113 | 80.369 | 1.00 | 27.33 | DIC |
| ATOM | 1056 | CG | GLU | 137 | 15.631 | 58.389 | 81.868 | 1.00 | 32.13 | DIC |
| ATOM | 1057 | CD | GLU | 137 | 15.115 | 57.184 | 82.658 | 1.00 | 34.75 | DIC |
| ATOM | 1058 | OE1 | GLU | 137 | 14.132 | 56.543 | 82.217 | 1.00 | 36.51 | DIC |
| ATOM | 1059 | OE2 | GLU | 137 | 15.684 | 56.884 | 83.735 | 1.00 | 35.95 | DIC |
| ATOM | 1060 | C | GLU | 137 | 16.428 | 55.688 | 80.342 | 1.00 | 22.27 | DIC |
| ATOM | 1061 | O | GLU | 137 | 15.508 | 55.194 | 79.687 | 1.00 | 23.05 | DIC |
| ATOM | 1062 | N | GLY | 138 | 17.029 | 55.058 | 81.346 | 1.00 | 21.87 | DIC |
| ATOM | 1063 | CA | GLY | 138 | 16.604 | 53.738 | 81.774 | 1.00 | 20.02 | DIC |
| ATOM | 1064 | C | GLY | 138 | 16.855 | 52.556 | 80.859 | 1.00 | 19.14 | DIC |
| ATOM | 1065 | O | GLY | 138 | 16.587 | 51.426 | 81.253 | 1.00 | 19.29 | DIC |
| ATOM | 1066 | N | TYR | 139 | 17.371 | 52.779 | 79.654 | 1.00 | 18.23 | DIC |
| ATOM | 1067 | CA | TYR | 139 | 17.605 | 51.643 | 78.762 | 1.00 | 17.46 | DIC |
| ATOM | 1068 | CB | TYR | 139 | 18.111 | 52.080 | 77.389 | 1.00 | 15.54 | DIC |
| ATOM | 1069 | CG | TYR | 139 | 18.398 | 50.877 | 76.513 | 1.00 | 14.37 | DIC |
| ATOM | 1070 | CD1 | TYR | 139 | 17.358 | 50.087 | 76.029 | 1.00 | 14.45 | DIC |
| ATOM | 1071 | CE1 | TYR | 139 | 17.604 | 48.932 | 75.298 | 1.00 | 14.12 | DIC |
| ATOM | 1072 | CD2 | TYR | 139 | 19.708 | 50.480 | 76.237 | 1.00 | 14.19 | DIC |
| ATOM | 1073 | CE2 | TYR | 139 | 19.969 | 49.313 | 75.503 | 1.00 | 13.62 | DIC |
| ATOM | 1074 | CZ | TYR | 139 | 18.910 | 48.547 | 75.039 | 1.00 | 13.67 | DIC |
| ATOM | 1075 | OH | TYR | 139 | 19.142 | 47.398 | 74.316 | 1.00 | 12.91 | DIC |
| ATOM | 1076 | C | TYR | 139 | 18.621 | 50.664 | 79.326 | 1.00 | 16.80 | DIC |
| ATOM | 1077 | O | TYR | 139 | 18.353 | 49.474 | 79.442 | 1.00 | 18.25 | DIC |
| ATOM | 1078 | N | LYS | 140 | 19.798 | 51.183 | 79.638 | 1.00 | 15.97 | DIC |
| ATOM | 1079 | CA | LYS | 140 | 20.905 | 50.398 | 80.166 | 1.00 | 16.47 | DIC |
| ATOM | 1080 | CB | LYS | 140 | 21.957 | 51.356 | 80.726 | 1.00 | 17.96 | DIC |
| ATOM | 1081 | CG | LYS | 140 | 23.344 | 50.799 | 80.841 | 1.00 | 18.12 | DIC |
| ATOM | 1082 | CD | LYS | 140 | 24.340 | 51.897 | 81.191 | 1.00 | 17.26 | DIC |
| ATOM | 1083 | CE | LYS | 140 | 24.014 | 52.584 | 82.505 | 1.00 | 18.38 | DIC |
| ATOM | 1084 | NZ | LYS | 140 | 25.094 | 53.524 | 82.900 | 1.00 | 18.19 | DIC |
| ATOM | 1085 | C | LYS | 140 | 20.443 | 49.427 | 81.244 | 1.00 | 17.24 | DIC |
| ATOM | 1086 | O | LYS | 140 | 20.853 | 48.264 | 81.264 | 1.00 | 16.57 | DIC |
| ATOM | 1087 | N | GLN | 141 | 19.579 | 49.907 | 82.134 | 1.00 | 16.44 | DIC |
| ATOM | 1088 | CA | GLN | 141 | 19.064 | 49.086 | 83.219 | 1.00 | 17.66 | DIC |
| ATOM | 1089 | CB | GLN | 141 | 18.298 | 49.958 | 84.220 | 1.00 | 17.58 | DIC |
| ATOM | 1090 | CG | GLN | 141 | 19.177 | 50.921 | 85.010 | 1.00 | 16.44 | DIC |
| ATOM | 1091 | CD | GLN | 141 | 19.667 | 52.100 | 84.183 | 1.00 | 18.00 | DIC |
| ATOM | 1092 | OE1 | GLN | 141 | 19.272 | 52.274 | 83.033 | 1.00 | 17.29 | DIC |
| ATOM | 1093 | NE2 | GLN | 141 | 20.525 | 52.920 | 84.772 | 1.00 | 17.45 | DIC |
| ATOM | 1094 | C | GLN | 141 | 18.176 | 47.941 | 82.730 | 1.00 | 17.85 | DIC |
| ATOM | 1095 | O | GLN | 141 | 18.076 | 46.902 | 83.384 | 1.00 | 18.70 | DIC |
| ATOM | 1096 | N | LYS | 142 | 17.534 | 48.130 | 81.582 | 1.00 | 18.33 | DIC |
| ATOM | 1097 | CA | LYS | 142 | 16.678 | 47.094 | 81.007 | 1.00 | 18.53 | DIC |
| ATOM | 1098 | CB | LYS | 142 | 15.989 | 47.600 | 79.736 | 1.00 | 20.13 | DIC |
| ATOM | 1099 | CG | LYS | 142 | 14.819 | 48.553 | 79.963 | 1.00 | 23.26 | DIC |
| ATOM | 1100 | CD | LYS | 142 | 14.382 | 49.182 | 78.639 | 1.00 | 24.62 | DIC |
| ATOM | 1101 | CE | LYS | 142 | 13.112 | 50.012 | 78.779 | 1.00 | 27.12 | DIC |
| ATOM | 1102 | NZ | LYS | 142 | 11.922 | 49.174 | 79.116 | 1.00 | 27.95 | DIC |
| ATOM | 1103 | C | LYS | 142 | 17.477 | 45.839 | 80.662 | 1.00 | 17.52 | DIC |
| ATOM | 1104 | O | LYS | 142 | 16.921 | 44.737 | 80.628 | 1.00 | 17.09 | DIC |
| ATOM | 1105 | N | ILE | 143 | 18.771 | 45.987 | 80.384 | 1.00 | 15.77 | DIC |
| ATOM | 1106 | CA | ILE | 143 | 19.561 | 44.802 | 80.054 | 1.00 | 13.94 | DIC |
| ATOM | 1107 | CB | ILE | 143 | 20.391 | 44.979 | 78.754 | 1.00 | 13.64 | DIC |
| ATOM | 1108 | CG2 | ILE | 143 | 19.467 | 45.307 | 77.584 | 1.00 | 13.27 | DIC |
| ATOM | 1109 | CG1 | ILE | 143 | 21.434 | 46.079 | 78.919 | 1.00 | 13.02 | DIC |
| ATOM | 1110 | CD | ILE | 143 | 22.390 | 46.147 | 77.731 | 1.00 | 13.03 | DIC |
| ATOM | 1111 | C | ILE | 143 | 20.472 | 44.349 | 81.193 | 1.00 | 13.33 | DIC |
| ATOM | 1112 | O | ILE | 143 | 21.480 | 43.679 | 80.974 | 1.00 | 12.63 | DIC |
| ATOM | 1113 | N | GLY | 144 | 20.099 | 44.722 | 82.415 | 1.00 | 13.50 | DIC |
| ATOM | 1114 | CA | GLY | 144 | 20.849 | 44.304 | 83.585 | 1.00 | 12.47 | DIC |
| ATOM | 1115 | C | GLY | 144 | 22.073 | 45.084 | 84.013 | 1.00 | 13.88 | DIC |
| ATOM | 1116 | O | GLY | 144 | 22.794 | 44.632 | 84.903 | 1.00 | 12.20 | DIC |
| ATOM | 1117 | N | MSE | 145 | 22.320 | 46.242 | 83.410 | 1.00 | 14.02 | DIC |
| ATOM | 1118 | CA | MSE | 145 | 23.487 | 47.028 | 83.789 | 1.00 | 15.34 | DIC |
| ATOM | 1119 | CB | MSE | 145 | 24.054 | 47.746 | 82.568 | 1.00 | 14.88 | DIC |
| ATOM | 1120 | CG | MSE | 145 | 24.544 | 46.810 | 81.471 | 1.00 | 14.27 | DIC |
| ATOM | 1121 | SE | MSE | 145 | 25.198 | 47.793 | 79.939 | 1.00 | 16.53 | DIC |
| ATOM | 1122 | CE | MSE | 145 | 26.784 | 48.563 | 80.704 | 1.00 | 12.51 | DIC |
| ATOM | 1123 | C | MSE | 145 | 23.166 | 48.045 | 84.882 | 1.00 | 16.35 | DIC |
| ATOM | 1124 | O | MSE | 145 | 22.033 | 48.516 | 84.996 | 1.00 | 16.93 | DIC |
| ATOM | 1125 | N | ALA | 146 | 24.168 | 48.379 | 85.687 | 1.00 | 17.18 | DIC |
| ATOM | 1126 | CA | ALA | 146 | 23.979 | 49.357 | 86.760 | 1.00 | 17.93 | DIC |
| ATOM | 1127 | CB | ALA | 146 | 24.876 | 49.016 | 87.947 | 1.00 | 19.51 | DIC |
| ATOM | 1128 | C | ALA | 146 | 24.304 | 50.753 | 86.235 | 1.00 | 18.51 | DIC |
| ATOM | 1129 | O | ALA | 146 | 24.880 | 50.892 | 85.156 | 1.00 | 16.64 | DIC |
| ATOM | 1130 | N | ASP | 147 | 23.929 | 51.782 | 86.995 | 1.00 | 18.52 | DIC |
| ATOM | 1131 | CA | ASP | 147 | 24.181 | 53.166 | 86.594 | 1.00 | 19.15 | DIC |
| ATOM | 1132 | CB | ASP | 147 | 23.589 | 54.143 | 87.622 | 1.00 | 22.16 | DIC |
| ATOM | 1133 | CG | ASP | 147 | 23.911 | 55.600 | 87.299 | 1.00 | 24.53 | DIC |
| ATOM | 1134 | OD1 | ASP | 147 | 23.468 | 56.101 | 86.245 | 1.00 | 27.39 | DIC |
| ATOM | 1135 | OD2 | ASP | 147 | 24.614 | 56.250 | 88.096 | 1.00 | 26.51 | DIC |
| ATOM | 1136 | C | ASP | 147 | 25.668 | 53.456 | 86.427 | 1.00 | 18.18 | DIC |
| ATOM | 1137 | O | ASP | 147 | 26.062 | 54.243 | 85.558 | 1.00 | 18.44 | DIC |
| ATOM | 1138 | N | GLY | 148 | 26.485 | 52.813 | 87.258 | 1.00 | 15.95 | DIC |
| ATOM | 1139 | CA | GLY | 148 | 27.920 | 53.015 | 87.207 | 1.00 | 14.91 | DIC |
| ATOM | 1140 | C | GLY | 148 | 28.645 | 52.201 | 86.152 | 1.00 | 14.80 | DIC |
| ATOM | 1141 | O | GLY | 148 | 29.858 | 52.324 | 85.996 | 1.00 | 14.63 | DIC |
| ATOM | 1142 | N | GLU | 149 | 27.912 | 51.360 | 85.430 | 1.00 | 13.75 | DIC |
| ATOM | 1143 | CA | GLU | 149 | 28.510 | 50.548 | 84.379 | 1.00 | 14.04 | DIC |
| ATOM | 1144 | CB | GLU | 149 | 27.953 | 49.121 | 84.424 | 1.00 | 13.92 | DIC |
| ATOM | 1145 | CG | GLU | 149 | 28.202 | 48.416 | 85.755 | 1.00 | 14.93 | DIC |
| ATOM | 1146 | CD | GLU | 149 | 27.689 | 46.989 | 85.769 | 1.00 | 14.83 | DIC |
| ATOM | 1147 | OE1 | GLU | 149 | 26.585 | 46.749 | 85.238 | 1.00 | 15.36 | DIC |
| ATOM | 1148 | OE2 | GLU | 149 | 28.383 | 46.109 | 86.320 | 1.00 | 14.29 | DIC |
| ATOM | 1149 | C | GLU | 149 | 28.150 | 51.237 | 83.067 | 1.00 | 13.46 | DIC |
| ATOM | 1150 | O | GLU | 149 | 26.980 | 51.361 | 82.716 | 1.00 | 13.23 | DIC |
| ATOM | 1151 | N | TYR | 150 | 29.165 | 51.695 | 82.349 | 1.00 | 12.32 | DIC |
| ATOM | 1152 | CA | TYR | 150 | 28.928 | 52.419 | 81.116 | 1.00 | 12.18 | DIC |
| ATOM | 1153 | CB | TYR | 150 | 30.023 | 53.470 | 80.961 | 1.00 | 12.48 | DIC |
| ATOM | 1154 | CG | TYR | 150 | 30.127 | 54.320 | 82.211 | 1.00 | 13.07 | DIC |
| ATOM | 1155 | CD1 | TYR | 150 | 31.351 | 54.526 | 82.833 | 1.00 | 12.79 | DIC |
| ATOM | 1156 | CE1 | TYR | 150 | 31.442 | 55.235 | 84.016 | 1.00 | 13.05 | DIC |
| ATOM | 1157 | CD2 | TYR | 150 | 28.984 | 54.860 | 82.807 | 1.00 | 12.92 | DIC |
| ATOM | 1158 | CE2 | TYR | 150 | 29.063 | 55.577 | 83.999 | 1.00 | 13.33 | DIC |
| ATOM | 1159 | CZ | TYR | 150 | 30.302 | 55.756 | 84.595 | 1.00 | 14.14 | DIC |
| ATOM | 1160 | OH | TYR | 150 | 30.417 | 56.439 | 85.775 | 1.00 | 13.21 | DIC |
| ATOM | 1161 | C | TYR | 150 | 28.786 | 51.562 | 79.870 | 1.00 | 10.46 | DIC |
| ATOM | 1162 | O | TYR | 150 | 29.604 | 50.681 | 79.603 | 1.00 | 9.61 | DIC |
| ATOM | 1163 | N | TYR | 151 | 27.720 | 51.839 | 79.126 | 1.00 | 9.17 | DIC |
| ATOM | 1164 | CA | TYR | 151 | 27.381 | 51.124 | 77.901 | 1.00 | 8.84 | DIC |
| ATOM | 1165 | CB | TYR | 151 | 25.949 | 51.469 | 77.502 | 1.00 | 8.39 | DIC |
| ATOM | 1166 | CG | TYR | 151 | 25.359 | 50.630 | 76.391 | 1.00 | 9.04 | DIC |
| ATOM | 1167 | CD1 | TYR | 151 | 25.744 | 50.817 | 75.065 | 1.00 | 8.73 | DIC |
| ATOM | 1168 | CE1 | TYR | 151 | 25.175 | 50.062 | 74.039 | 1.00 | 8.50 | DIC |
| ATOM | 1169 | CD2 | TYR | 151 | 24.387 | 49.666 | 76.668 | 1.00 | 8.78 | DIC |
| ATOM | 1170 | CE2 | TYR | 151 | 23.813 | 48.910 | 75.649 | 1.00 | 8.51 | DIC |
| ATOM | 1171 | CZ | TYR | 151 | 24.212 | 49.113 | 74.339 | 1.00 | 8.79 | DIC |
| ATOM | 1172 | OH | TYR | 151 | 23.650 | 48.364 | 73.331 | 1.00 | 6.56 | DIC |
| ATOM | 1173 | C | TYR | 151 | 28.346 | 51.519 | 76.797 | 1.00 | 7.98 | DIC |
| ATOM | 1174 | O | TYR | 151 | 28.461 | 52.697 | 76.456 | 1.00 | 8.75 | DIC |
| ATOM | 1175 | N | PHE | 152 | 29.048 | 50.538 | 76.239 | 1.00 | 7.59 | DIC |
| ATOM | 1176 | CA | PHE | 152 | 30.008 | 50.839 | 75.192 | 1.00 | 8.73 | DIC |
| ATOM | 1177 | CB | PHE | 152 | 31.315 | 50.076 | 75.426 | 1.00 | 9.12 | DIC |
| ATOM | 1178 | CG | PHE | 152 | 31.165 | 48.581 | 75.416 | 1.00 | 9.52 | DIC |
| ATOM | 1179 | CD1 | PHE | 152 | 31.025 | 47.870 | 76.605 | 1.00 | 10.37 | DIC |
| ATOM | 1180 | CD2 | PHE | 152 | 31.206 | 47.875 | 74.219 | 1.00 | 9.21 | DIC |
| ATOM | 1181 | CE1 | PHE | 152 | 30.933 | 46.470 | 76.601 | 1.00 | 10.52 | DIC |
| ATOM | 1182 | CE2 | PHE | 152 | 31.115 | 46.474 | 74.208 | 1.00 | 10.71 | DIC |
| ATOM | 1183 | CZ | PHE | 152 | 30.980 | 45.775 | 75.397 | 1.00 | 10.62 | DIC |
| ATOM | 1184 | C | PHE | 152 | 29.468 | 50.530 | 73.810 | 1.00 | 9.25 | DIC |
| ATOM | 1185 | O | PHE | 152 | 28.521 | 49.753 | 73.659 | 1.00 | 9.63 | DIC |
| ATOM | 1186 | N | ASN | 153 | 30.060 | 51.169 | 72.804 | 1.00 | 9.40 | DIC |
| ATOM | 1187 | CA | ASN | 153 | 29.661 | 50.955 | 71.422 | 1.00 | 8.94 | DIC |
| ATOM | 1188 | CB | ASN | 153 | 29.916 | 52.211 | 70.590 | 1.00 | 10.41 | DIC |
| ATOM | 1189 | CG | ASN | 153 | 29.541 | 52.025 | 69.136 | 1.00 | 10.47 | DIC |
| ATOM | 1190 | OD1 | ASN | 153 | 30.337 | 51.537 | 68.334 | 1.00 | 11.66 | DIC |
| ATOM | 1191 | ND2 | ASN | 153 | 28.312 | 52.391 | 68.794 | 1.00 | 11.08 | DIC |
| ATOM | 1192 | C | ASN | 153 | 30.492 | 49.795 | 70.910 | 1.00 | 8.26 | DIC |
| ATOM | 1193 | O | ASN | 153 | 31.691 | 49.724 | 71.174 | 1.00 | 6.77 | DIC |
| ATOM | 1194 | N | ALA | 154 | 29.856 | 48.885 | 70.180 | 1.00 | 7.02 | DIC |
| ATOM | 1195 | CA | ALA | 154 | 30.547 | 47.702 | 69.676 | 1.00 | 7.92 | DIC |
| ATOM | 1196 | CB | ALA | 154 | 29.551 | 46.551 | 69.529 | 1.00 | 6.96 | DIC |
| ATOM | 1197 | C | ALA | 154 | 31.300 | 47.902 | 68.365 | 1.00 | 7.01 | DIC |
| ATOM | 1198 | O | ALA | 154 | 31.819 | 46.938 | 67.795 | 1.00 | 7.48 | DIC |
| ATOM | 1199 | N | GLY | 155 | 31.376 | 49.143 | 67.901 | 1.00 | 6.72 | DIC |
| ATOM | 1200 | CA | GLY | 155 | 32.053 | 49.426 | 66.647 | 1.00 | 7.21 | DIC |
| ATOM | 1201 | C | GLY | 155 | 33.573 | 49.409 | 66.635 | 1.00 | 7.94 | DIC |
| ATOM | 1202 | O | GLY | 155 | 34.185 | 49.242 | 65.575 | 1.00 | 7.30 | DIC |
| ATOM | 1203 | N | VAL | 156 | 34.183 | 49.606 | 67.800 | 1.00 | 7.67 | DIC |
| ATOM | 1204 | CA | VAL | 156 | 35.634 | 49.604 | 67.930 | 1.00 | 6.73 | DIC |
| ATOM | 1205 | CB | VAL | 156 | 36.183 | 51.053 | 68.035 | 1.00 | 8.07 | DIC |
| ATOM | 1206 | CG1 | VAL | 156 | 37.705 | 51.044 | 68.190 | 1.00 | 8.55 | DIC |
| ATOM | 1207 | CG2 | VAL | 156 | 35.794 | 51.846 | 66.784 | 1.00 | 6.66 | DIC |
| ATOM | 1208 | C | VAL | 156 | 35.928 | 48.834 | 69.210 | 1.00 | 6.86 | DIC |
| ATOM | 1209 | O | VAL | 156 | 35.639 | 49.306 | 70.304 | 1.00 | 5.93 | DIC |
| ATOM | 1210 | N | LEU | 157 | 36.493 | 47.640 | 69.066 | 1.00 | 5.63 | DIC |
| ATOM | 1211 | CA | LEU | 157 | 36.789 | 46.791 | 70.215 | 1.00 | 6.45 | DIC |
| ATOM | 1212 | CB | LEU | 157 | 35.818 | 45.607 | 70.246 | 1.00 | 5.76 | DIC |
| ATOM | 1213 | CG | LEU | 157 | 34.328 | 45.916 | 70.075 | 1.00 | 5.74 | DIC |
| ATOM | 1214 | CD1 | LEU | 157 | 33.585 | 44.625 | 69.840 | 1.00 | 4.70 | DIC |
| ATOM | 1215 | CD2 | LEU | 157 | 33.783 | 46.662 | 71.291 | 1.00 | 3.22 | DIC |
| ATOM | 1216 | C | LEU | 157 | 38.195 | 46.231 | 70.244 | 1.00 | 7.54 | DIC |
| ATOM | 1217 | O | LEU | 157 | 38.614 | 45.559 | 69.301 | 1.00 | 8.36 | DIC |
| ATOM | 1218 | N | LEU | 158 | 38.927 | 46.509 | 71.320 | 1.00 | 8.42 | DIC |
| ATOM | 1219 | CA | LEU | 158 | 40.264 | 45.947 | 71.486 | 1.00 | 9.61 | DIC |
| ATOM | 1220 | CB | LEU | 158 | 41.177 | 46.886 | 72.285 | 1.00 | 10.49 | DIC |
| ATOM | 1221 | CG | LEU | 158 | 42.640 | 46.441 | 72.402 | 1.00 | 10.54 | DIC |
| ATOM | 1222 | CD1 | LEU | 158 | 43.281 | 46.378 | 71.022 | 1.00 | 11.43 | DIC |
| ATOM | 1223 | CD2 | LEU | 158 | 43.394 | 47.419 | 73.282 | 1.00 | 12.79 | DIC |
| ATOM | 1224 | C | LEU | 158 | 39.940 | 44.695 | 72.291 | 1.00 | 10.50 | DIC |
| ATOM | 1225 | O | LEU | 158 | 39.581 | 44.772 | 73.466 | 1.00 | 10.19 | DIC |
| ATOM | 1226 | N | ILE | 159 | 40.074 | 43.538 | 71.656 | 1.00 | 11.09 | DIC |
| ATOM | 1227 | CA | ILE | 159 | 39.703 | 42.284 | 72.287 | 1.00 | 11.44 | DIC |
| ATOM | 1228 | CB | ILE | 159 | 38.855 | 41.459 | 71.277 | 1.00 | 12.05 | DIC |
| ATOM | 1229 | CG2 | ILE | 159 | 38.540 | 40.069 | 71.821 | 1.00 | 11.71 | DIC |
| ATOM | 1230 | CG1 | ILE | 159 | 37.575 | 42.247 | 70.961 | 1.00 | 12.04 | DIC |
| ATOM | 1231 | CD | ILE | 159 | 36.693 | 41.637 | 69.916 | 1.00 | 13.91 | DIC |
| ATOM | 1232 | C | ILE | 159 | 40.817 | 41.422 | 72.877 | 1.00 | 12.11 | DIC |
| ATOM | 1233 | O | ILE | 159 | 41.846 | 41.179 | 72.253 | 1.00 | 10.85 | DIC |
| ATOM | 1234 | N | ASN | 160 | 40.594 | 40.974 | 74.108 | 1.00 | 12.74 | DIC |
| ATOM | 1235 | CA | ASN | 160 | 41.542 | 40.110 | 74.800 | 1.00 | 12.51 | DIC |
| ATOM | 1236 | CB | ASN | 160 | 41.325 | 40.243 | 76.309 | 1.00 | 12.70 | DIC |
| ATOM | 1237 | CG | ASN | 160 | 42.282 | 39.404 | 77.119 | 1.00 | 12.50 | DIC |
| ATOM | 1238 | OD1 | ASN | 160 | 42.920 | 38.492 | 76.600 | 1.00 | 12.15 | DIC |
| ATOM | 1239 | ND2 | ASN | 160 | 42.378 | 39.703 | 78.405 | 1.00 | 10.49 | DIC |
| ATOM | 1240 | C | ASN | 160 | 41.178 | 38.701 | 74.325 | 1.00 | 13.36 | DIC |
| ATOM | 1241 | O | ASN | 160 | 40.512 | 37.956 | 75.041 | 1.00 | 13.18 | DIC |
| ATOM | 1242 | N | LEU | 161 | 41.601 | 38.342 | 73.112 | 1.00 | 14.44 | DIC |
| ATOM | 1243 | CA | LEU | 161 | 41.266 | 37.032 | 72.552 | 1.00 | 15.68 | DIC |
| ATOM | 1244 | CB | LEU | 161 | 41.846 | 36.856 | 71.142 | 1.00 | 15.57 | DIC |
| ATOM | 1245 | CG | LEU | 161 | 40.891 | 37.070 | 69.962 | 1.00 | 17.77 | DIC |
| ATOM | 1246 | CD1 | LEU | 161 | 41.565 | 36.578 | 68.692 | 1.00 | 16.72 | DIC |
| ATOM | 1247 | CD2 | LEU | 161 | 39.576 | 36.321 | 70.180 | 1.00 | 16.73 | DIC |
| ATOM | 1248 | C | LEU | 161 | 41.696 | 35.858 | 73.407 | 1.00 | 16.42 | DIC |
| ATOM | 1249 | O | LEU | 161 | 40.977 | 34.865 | 73.500 | 1.00 | 15.59 | DIC |
| ATOM | 1250 | N | LYS | 162 | 42.877 | 35.955 | 74.007 | 1.00 | 17.58 | DIC |
| ATOM | 1251 | CA | LYS | 162 | 43.365 | 34.883 | 74.865 | 1.00 | 18.16 | DIC |
| ATOM | 1252 | CB | LYS | 162 | 44.683 | 35.297 | 75.528 | 1.00 | 20.45 | DIC |
| ATOM | 1253 | CG | LYS | 162 | 45.123 | 34.368 | 76.643 | 1.00 | 22.89 | DIC |
| ATOM | 1254 | CD | LYS | 162 | 46.599 | 34.511 | 76.971 | 1.00 | 24.41 | DIC |
| ATOM | 1255 | CE | LYS | 162 | 47.470 | 33.764 | 75.961 | 1.00 | 25.88 | DIC |
| ATOM | 1256 | NZ | LYS | 162 | 48.892 | 33.703 | 76.422 | 1.00 | 26.93 | DIC |
| ATOM | 1257 | C | LYS | 162 | 42.303 | 34.568 | 75.927 | 1.00 | 17.16 | DIC |
| ATOM | 1258 | O | LYS | 162 | 42.020 | 33.406 | 76.210 | 1.00 | 16.94 | DIC |
| ATOM | 1259 | N | LYS | 163 | 41.705 | 35.608 | 76.506 | 1.00 | 16.20 | DIC |
| ATOM | 1260 | CA | LYS | 163 | 40.670 | 35.407 | 77.514 | 1.00 | 13.83 | DIC |
| ATOM | 1261 | CB | LYS | 163 | 40.291 | 36.728 | 78.189 | 1.00 | 13.03 | DIC |
| ATOM | 1262 | CG | LYS | 163 | 39.242 | 36.563 | 79.297 | 1.00 | 13.52 | DIC |
| ATOM | 1263 | CD | LYS | 163 | 39.031 | 37.864 | 80.064 | 1.00 | 13.40 | DIC |
| ATOM | 1264 | CE | LYS | 163 | 37.998 | 37.714 | 81.172 | 1.00 | 13.29 | DIC |
| ATOM | 1265 | NZ | LYS | 163 | 38.407 | 36.647 | 82.124 | 1.00 | 16.98 | DIC |
| ATOM | 1266 | C | LYS | 163 | 39.428 | 34.792 | 76.877 | 1.00 | 13.30 | DIC |
| ATOM | 1267 | O | LYS | 163 | 38.870 | 33.826 | 77.394 | 1.00 | 13.39 | DIC |
| ATOM | 1268 | N | TRP | 164 | 38.995 | 35.352 | 75.751 | 1.00 | 12.69 | DIC |
| ATOM | 1269 | CA | TRP | 164 | 37.817 | 34.832 | 75.056 | 1.00 | 12.44 | DIC |
| ATOM | 1270 | CB | TRP | 164 | 37.608 | 35.582 | 73.743 | 1.00 | 11.58 | DIC |
| ATOM | 1271 | CG | TRP | 164 | 37.001 | 36.958 | 73.891 | 1.00 | 10.67 | DIC |
| ATOM | 1272 | CD2 | TRP | 164 | 36.032 | 37.560 | 73.025 | 1.00 | 10.33 | DIC |
| ATOM | 1273 | CE2 | TRP | 164 | 35.796 | 38.867 | 73.502 | 1.00 | 10.22 | DIC |
| ATOM | 1274 | CE3 | TRP | 164 | 35.341 | 37.119 | 71.886 | 1.00 | 10.89 | DIC |
| ATOM | 1275 | CD1 | TRP | 164 | 37.304 | 37.895 | 74.833 | 1.00 | 10.43 | DIC |
| ATOM | 1276 | NE1 | TRP | 164 | 36.584 | 39.048 | 74.607 | 1.00 | 9.98 | DIC |
| ATOM | 1277 | CZ2 | TRP | 164 | 34.896 | 39.743 | 72.880 | 1.00 | 10.17 | DIC |
| ATOM | 1278 | CZ3 | TRP | 164 | 34.446 | 37.989 | 71.270 | 1.00 | 9.91 | DIC |
| ATOM | 1279 | CH2 | TRP | 164 | 34.234 | 39.286 | 71.769 | 1.00 | 9.87 | DIC |
| ATOM | 1280 | C | TRP | 164 | 37.923 | 33.329 | 74.766 | 1.00 | 13.17 | DIC |
| ATOM | 1281 | O | TRP | 164 | 36.961 | 32.580 | 74.959 | 1.00 | 10.76 | DIC |
| ATOM | 1282 | N | ARG | 165 | 39.090 | 32.882 | 74.311 | 1.00 | 13.97 | DIC |
| ATOM | 1283 | CA | ARG | 165 | 39.248 | 31.473 | 73.984 | 1.00 | 15.74 | DIC |
| ATOM | 1284 | CB | ARG | 165 | 40.576 | 31.242 | 73.250 | 1.00 | 16.55 | DIC |
| ATOM | 1285 | CG | ARG | 165 | 40.579 | 31.829 | 71.833 | 1.00 | 17.08 | DIC |
| ATOM | 1286 | CD | ARG | 165 | 41.687 | 31.245 | 70.956 | 1.00 | 16.86 | DIC |
| ATOM | 1287 | NE | ARG | 165 | 43.006 | 31.584 | 71.473 | 1.00 | 17.35 | DIC |
| ATOM | 1288 | CZ | ARG | 165 | 43.679 | 32.678 | 71.144 | 1.00 | 16.72 | DIC |
| ATOM | 1289 | NH1 | ARG | 165 | 44.865 | 32.907 | 71.678 | 1.00 | 17.09 | DIC |
| ATOM | 1290 | NH2 | ARG | 165 | 43.177 | 33.528 | 70.259 | 1.00 | 17.44 | DIC |
| ATOM | 1291 | C | ARG | 165 | 39.099 | 30.534 | 75.180 | 1.00 | 16.13 | DIC |
| ATOM | 1292 | O | ARG | 165 | 38.938 | 29.332 | 75.008 | 1.00 | 16.33 | DIC |
| ATOM | 1293 | N | ARG | 166 | 39.124 | 31.086 | 76.389 | 1.00 | 16.52 | DIC |
| ATOM | 1294 | CA | ARG | 166 | 38.959 | 30.282 | 77.597 | 1.00 | 17.40 | DIC |
| ATOM | 1295 | CB | ARG | 166 | 39.581 | 30.988 | 78.803 | 1.00 | 18.34 | DIC |
| ATOM | 1296 | CG | ARG | 166 | 41.089 | 31.074 | 78.767 | 1.00 | 20.01 | DIC |
| ATOM | 1297 | CD | ARG | 166 | 41.598 | 31.993 | 79.856 | 1.00 | 21.20 | DIC |
| ATOM | 1298 | NE | ARG | 166 | 43.045 | 32.146 | 79.773 | 1.00 | 23.88 | DIC |
| ATOM | 1299 | CZ | ARG | 166 | 43.688 | 33.305 | 79.868 | 1.00 | 24.44 | DIC |
| ATOM | 1300 | NH1 | ARG | 166 | 43.015 | 34.436 | 80.051 | 1.00 | 24.80 | DIC |
| ATOM | 1301 | NH2 | ARG | 166 | 45.010 | 33.334 | 79.770 | 1.00 | 24.70 | DIC |
| ATOM | 1302 | C | ARG | 166 | 37.480 | 30.033 | 77.889 | 1.00 | 17.20 | DIC |
| ATOM | 1303 | O | ARG | 166 | 37.145 | 29.220 | 78.745 | 1.00 | 16.99 | DIC |
| ATOM | 1304 | N | HIS | 167 | 36.595 | 30.726 | 77.177 | 1.00 | 16.51 | DIC |
| ATOM | 1305 | CA | HIS | 167 | 35.164 | 30.572 | 77.403 | 1.00 | 15.83 | DIC |
| ATOM | 1306 | CB | HIS | 167 | 34.609 | 31.848 | 78.055 | 1.00 | 15.92 | DIC |
| ATOM | 1307 | CG | HIS | 167 | 35.323 | 32.253 | 79.311 | 1.00 | 16.65 | DIC |
| ATOM | 1308 | CD2 | HIS | 167 | 36.463 | 32.960 | 79.499 | 1.00 | 16.23 | DIC |
| ATOM | 1309 | ND1 | HIS | 167 | 34.866 | 31.924 | 80.570 | 1.00 | 15.62 | DIC |
| ATOM | 1310 | CE1 | HIS | 167 | 35.693 | 32.412 | 81.478 | 1.00 | 16.49 | DIC |
| ATOM | 1311 | NE2 | HIS | 167 | 36.670 | 33.046 | 80.854 | 1.00 | 15.49 | DIC |
| ATOM | 1312 | C | HIS | 167 | 34.397 | 30.288 | 76.106 | 1.00 | 15.86 | DIC |
| ATOM | 1313 | O | HIS | 167 | 34.953 | 30.360 | 75.005 | 1.00 | 16.09 | DIC |
| ATOM | 1314 | N | ASP | 168 | 33.118 | 29.956 | 76.248 | 1.00 | 14.42 | DIC |
| ATOM | 1315 | CA | ASP | 168 | 32.261 | 29.687 | 75.097 | 1.00 | 14.22 | DIC |
| ATOM | 1316 | CB | ASP | 168 | 31.425 | 28.416 | 75.310 | 1.00 | 13.99 | DIC |
| ATOM | 1317 | CG | ASP | 168 | 30.692 | 27.981 | 74.049 | 1.00 | 13.84 | DIC |
| ATOM | 1318 | OD1 | ASP | 168 | 30.323 | 26.791 | 73.944 | 1.00 | 14.98 | DIC |
| ATOM | 1319 | OD2 | ASP | 168 | 30.475 | 28.829 | 73.159 | 1.00 | 12.73 | DIC |
| ATOM | 1320 | C | ASP | 168 | 31.352 | 30.896 | 74.951 | 1.00 | 13.64 | DIC |
| ATOM | 1321 | O | ASP | 168 | 30.215 | 30.897 | 75.423 | 1.00 | 13.29 | DIC |
| ATOM | 1322 | N | ILE | 169 | 31.882 | 31.922 | 74.292 | 1.00 | 12.99 | DIC |
| ATOM | 1323 | CA | ILE | 169 | 31.187 | 33.188 | 74.070 | 1.00 | 12.79 | DIC |
| ATOM | 1324 | CB | ILE | 169 | 32.093 | 34.169 | 73.286 | 1.00 | 12.63 | DIC |
| ATOM | 1325 | CG2 | ILE | 169 | 31.461 | 35.565 | 73.260 | 1.00 | 12.96 | DIC |
| ATOM | 1326 | CG1 | ILE | 169 | 33.471 | 34.242 | 73.952 | 1.00 | 12.18 | DIC |
| ATOM | 1327 | CD | ILE | 169 | 33.437 | 34.760 | 75.384 | 1.00 | 12.46 | DIC |
| ATOM | 1328 | C | ILE | 169 | 29.848 | 33.051 | 73.342 | 1.00 | 12.16 | DIC |
| ATOM | 1329 | O | ILE | 169 | 28.882 | 33.745 | 73.671 | 1.00 | 11.43 | DIC |
| ATOM | 1330 | N | PHE | 170 | 29.785 | 32.157 | 72.361 | 1.00 | 12.91 | DIC |
| ATOM | 1331 | CA | PHE | 170 | 28.545 | 31.953 | 71.618 | 1.00 | 13.05 | DIC |
| ATOM | 1332 | CB | PHE | 170 | 28.789 | 31.044 | 70.407 | 1.00 | 13.59 | DIC |
| ATOM | 1333 | CG | PHE | 170 | 27.558 | 30.804 | 69.569 | 1.00 | 13.99 | DIC |
| ATOM | 1334 | CD1 | PHE | 170 | 26.952 | 29.549 | 69.536 | 1.00 | 15.41 | DIC |
| ATOM | 1335 | CD2 | PHE | 170 | 26.987 | 31.839 | 68.838 | 1.00 | 14.92 | DIC |
| ATOM | 1336 | CE1 | PHE | 170 | 25.782 | 29.327 | 68.779 | 1.00 | 16.57 | DIC |
| ATOM | 1337 | CE2 | PHE | 170 | 25.822 | 31.633 | 68.081 | 1.00 | 15.52 | DIC |
| ATOM | 1338 | CZ | PHE | 170 | 25.218 | 30.375 | 68.052 | 1.00 | 15.31 | DIC |
| ATOM | 1339 | C | PHE | 170 | 27.473 | 31.350 | 72.528 | 1.00 | 13.66 | DIC |
| ATOM | 1340 | O | PHE | 170 | 26.303 | 31.756 | 72.491 | 1.00 | 13.17 | DIC |
| ATOM | 1341 | N | LYS | 171 | 27.871 | 30.392 | 73.358 | 1.00 | 13.51 | DIC |
| ATOM | 1342 | CA | LYS | 171 | 26.927 | 29.761 | 74.267 | 1.00 | 14.35 | DIC |
| ATOM | 1343 | CB | LYS | 171 | 27.601 | 28.601 | 75.009 | 1.00 | 16.69 | DIC |
| ATOM | 1344 | CG | LYS | 171 | 26.639 | 27.736 | 75.820 | 1.00 | 19.33 | DIC |
| ATOM | 1345 | CD | LYS | 171 | 27.224 | 26.351 | 76.092 | 1.00 | 22.68 | DIC |
| ATOM | 1346 | CE | LYS | 171 | 27.426 | 25.569 | 74.799 | 1.00 | 24.24 | DIC |
| ATOM | 1347 | NZ | LYS | 171 | 28.095 | 24.259 | 75.028 | 1.00 | 27.40 | DIC |
| ATOM | 1348 | C | LYS | 171 | 26.422 | 30.826 | 75.243 | 1.00 | 13.97 | DIC |
| ATOM | 1349 | O | LYS | 171 | 25.219 | 30.987 | 75.429 | 1.00 | 13.72 | DIC |
| ATOM | 1350 | N | MSE | 172 | 27.346 | 31.565 | 75.846 | 1.00 | 12.85 | DIC |
| ATOM | 1351 | CA | MSE | 172 | 26.983 | 32.632 | 76.770 | 1.00 | 13.48 | DIC |
| ATOM | 1352 | CB | MSE | 172 | 28.248 | 33.312 | 77.300 | 1.00 | 14.61 | DIC |
| ATOM | 1353 | CG | MSE | 172 | 29.085 | 32.439 | 78.237 | 1.00 | 15.80 | DIC |
| ATOM | 1354 | SE | MSE | 172 | 30.883 | 33.124 | 78.407 | 1.00 | 17.35 | DIC |
| ATOM | 1355 | CE | MSE | 172 | 30.480 | 34.735 | 79.370 | 1.00 | 14.60 | DIC |
| ATOM | 1356 | C | MSE | 172 | 26.089 | 33.671 | 76.076 | 1.00 | 13.42 | DIC |
| ATOM | 1357 | O | MSE | 172 | 25.210 | 34.261 | 76.708 | 1.00 | 13.11 | DIC |
| ATOM | 1358 | N | SER | 173 | 26.317 | 33.890 | 74.781 | 1.00 | 11.96 | DIC |
| ATOM | 1359 | CA | SER | 173 | 25.529 | 34.862 | 74.013 | 1.00 | 12.42 | DIC |
| ATOM | 1360 | CB | SER | 173 | 26.161 | 35.115 | 72.641 | 1.00 | 12.29 | DIC |
| ATOM | 1361 | OG | SER | 173 | 27.392 | 35.813 | 72.743 | 1.00 | 11.94 | DIC |
| ATOM | 1362 | C | SER | 173 | 24.097 | 34.376 | 73.813 | 1.00 | 12.80 | DIC |
| ATOM | 1363 | O | SER | 173 | 23.137 | 35.135 | 73.994 | 1.00 | 12.03 | DIC |
| ATOM | 1364 | N | SER | 174 | 23.966 | 33.109 | 73.429 | 1.00 | 12.50 | DIC |
| ATOM | 1365 | CA | SER | 174 | 22.663 | 32.504 | 73.200 | 1.00 | 12.95 | DIC |
| ATOM | 1366 | CB | SER | 174 | 22.816 | 31.058 | 72.726 | 1.00 | 12.51 | DIC |
| ATOM | 1367 | OG | SER | 174 | 23.397 | 31.000 | 71.439 | 1.00 | 14.05 | DIC |
| ATOM | 1368 | C | SER | 174 | 21.825 | 32.531 | 74.467 | 1.00 | 13.04 | DIC |
| ATOM | 1369 | O | SER | 174 | 20.634 | 32.843 | 74.422 | 1.00 | 11.55 | DIC |
| ATOM | 1370 | N | GLU | 175 | 22.444 | 32.187 | 75.593 | 1.00 | 13.58 | DIC |
| ATOM | 1371 | CA | GLU | 175 | 21.734 | 32.178 | 76.862 | 1.00 | 13.73 | DIC |
| ATOM | 1372 | CB | GLU | 175 | 22.609 | 31.558 | 77.957 | 1.00 | 15.98 | DIC |
| ATOM | 1373 | CG | GLU | 175 | 23.177 | 30.204 | 77.547 | 1.00 | 18.50 | DIC |
| ATOM | 1374 | CD | GLU | 175 | 23.988 | 29.525 | 78.638 | 1.00 | 20.33 | DIC |
| ATOM | 1375 | OE1 | GLU | 175 | 24.647 | 30.230 | 79.434 | 1.00 | 20.34 | DIC |
| ATOM | 1376 | OE2 | GLU | 175 | 23.977 | 28.272 | 78.681 | 1.00 | 21.97 | DIC |
| ATOM | 1377 | C | GLU | 175 | 21.332 | 33.598 | 77.238 | 1.00 | 12.97 | DIC |
| ATOM | 1378 | O | GLU | 175 | 20.244 | 33.818 | 77.762 | 1.00 | 12.29 | DIC |
| ATOM | 1379 | N | TRP | 176 | 22.199 | 34.568 | 76.954 | 1.00 | 12.77 | DIC |
| ATOM | 1380 | CA | TRP | 176 | 21.880 | 35.955 | 77.276 | 1.00 | 13.08 | DIC |
| ATOM | 1381 | CB | TRP | 176 | 23.105 | 36.853 | 77.085 | 1.00 | 13.64 | DIC |
| ATOM | 1382 | CG | TRP | 176 | 22.948 | 38.199 | 77.725 | 1.00 | 12.77 | DIC |
| ATOM | 1383 | CD2 | TRP | 176 | 22.404 | 39.376 | 77.125 | 1.00 | 12.50 | DIC |
| ATOM | 1384 | CE2 | TRP | 176 | 22.415 | 40.390 | 78.107 | 1.00 | 13.44 | DIC |
| ATOM | 1385 | CE3 | TRP | 176 | 21.904 | 39.674 | 75.852 | 1.00 | 12.57 | DIC |
| ATOM | 1386 | CD1 | TRP | 176 | 23.261 | 38.537 | 79.011 | 1.00 | 14.02 | DIC |
| ATOM | 1387 | NE1 | TRP | 176 | 22.945 | 39.849 | 79.249 | 1.00 | 13.70 | DIC |
| ATOM | 1388 | CZ2 | TRP | 176 | 21.942 | 41.685 | 77.855 | 1.00 | 13.81 | DIC |
| ATOM | 1389 | CZ3 | TRP | 176 | 21.432 | 40.962 | 75.601 | 1.00 | 11.62 | DIC |
| ATOM | 1390 | CH2 | TRP | 176 | 21.456 | 41.949 | 76.598 | 1.00 | 13.55 | DIC |
| ATOM | 1391 | C | TRP | 176 | 20.732 | 36.450 | 76.394 | 1.00 | 13.25 | DIC |
| ATOM | 1392 | O | TRP | 176 | 19.836 | 37.154 | 76.866 | 1.00 | 13.29 | DIC |
| ATOM | 1393 | N | VAL | 177 | 20.761 | 36.085 | 75.114 | 1.00 | 13.68 | DIC |
| ATOM | 1394 | CA | VAL | 177 | 19.708 | 36.493 | 74.188 | 1.00 | 13.26 | DIC |
| ATOM | 1395 | CB | VAL | 177 | 20.012 | 36.021 | 72.750 | 1.00 | 12.93 | DIC |
| ATOM | 1396 | CG1 | VAL | 177 | 18.760 | 36.146 | 71.874 | 1.00 | 12.53 | DIC |
| ATOM | 1397 | CG2 | VAL | 177 | 21.128 | 36.862 | 72.155 | 1.00 | 12.01 | DIC |
| ATOM | 1398 | C | VAL | 177 | 18.378 | 35.906 | 74.649 | 1.00 | 15.29 | DIC |
| ATOM | 1399 | O | VAL | 177 | 17.327 | 36.554 | 74.580 | 1.00 | 14.53 | DIC |
| ATOM | 1400 | N | GLU | 178 | 18.428 | 34.676 | 75.139 | 1.00 | 16.30 | DIC |
| ATOM | 1401 | CA | GLU | 178 | 17.221 | 34.021 | 75.607 | 1.00 | 19.15 | DIC |
| ATOM | 1402 | CB | GLU | 178 | 17.537 | 32.573 | 76.001 | 1.00 | 21.26 | DIC |
| ATOM | 1403 | CG | GLU | 178 | 16.313 | 31.704 | 76.275 | 1.00 | 25.37 | DIC |
| ATOM | 1404 | CD | GLU | 178 | 15.292 | 31.740 | 75.144 | 1.00 | 26.83 | DIC |
| ATOM | 1405 | OE1 | GLU | 178 | 15.687 | 31.622 | 73.960 | 1.00 | 28.31 | DIC |
| ATOM | 1406 | OE2 | GLU | 178 | 14.087 | 31.883 | 75.445 | 1.00 | 28.46 | DIC |
| ATOM | 1407 | C | GLU | 178 | 16.656 | 34.792 | 76.798 | 1.00 | 18.93 | DIC |
| ATOM | 1408 | O | GLU | 178 | 15.442 | 34.896 | 76.964 | 1.00 | 19.05 | DIC |
| ATOM | 1409 | N | GLN | 179 | 17.544 | 35.360 | 77.605 | 1.00 | 18.25 | DIC |
| ATOM | 1410 | CA | GLN | 179 | 17.141 | 36.105 | 78.790 | 1.00 | 20.06 | DIC |
| ATOM | 1411 | CB | GLN | 179 | 18.308 | 36.134 | 79.784 | 1.00 | 21.74 | DIC |
| ATOM | 1412 | CG | GLN | 179 | 18.013 | 36.801 | 81.119 | 1.00 | 25.00 | DIC |
| ATOM | 1413 | CD | GLN | 179 | 19.274 | 37.041 | 81.937 | 1.00 | 27.00 | DIC |
| ATOM | 1414 | OE1 | GLN | 179 | 19.221 | 37.576 | 83.050 | 1.00 | 29.16 | DIC |
| ATOM | 1415 | NE2 | GLN | 179 | 20.416 | 36.651 | 81.386 | 1.00 | 27.67 | DIC |
| ATOM | 1416 | C | GLN | 179 | 16.655 | 37.536 | 78.538 | 1.00 | 19.68 | DIC |
| ATOM | 1417 | O | GLN | 179 | 15.815 | 38.044 | 79.279 | 1.00 | 19.99 | DIC |
| ATOM | 1418 | N | TYR | 180 | 17.178 | 38.189 | 77.503 | 1.00 | 19.03 | DIC |
| ATOM | 1419 | CA | TYR | 180 | 16.799 | 39.570 | 77.217 | 1.00 | 18.49 | DIC |
| ATOM | 1420 | CB | TYR | 180 | 18.011 | 40.483 | 77.409 | 1.00 | 17.86 | DIC |
| ATOM | 1421 | CG | TYR | 180 | 18.475 | 40.591 | 78.840 | 1.00 | 16.21 | DIC |
| ATOM | 1422 | CD1 | TYR | 180 | 19.408 | 39.703 | 79.362 | 1.00 | 16.33 | DIC |
| ATOM | 1423 | CE1 | TYR | 180 | 19.840 | 39.811 | 80.679 | 1.00 | 16.04 | DIC |
| ATOM | 1424 | CD2 | TYR | 180 | 17.979 | 41.586 | 79.672 | 1.00 | 15.98 | DIC |
| ATOM | 1425 | CE2 | TYR | 180 | 18.400 | 41.698 | 80.988 | 1.00 | 15.72 | DIC |
| ATOM | 1426 | CZ | TYR | 180 | 19.330 | 40.812 | 81.485 | 1.00 | 15.22 | DIC |
| ATOM | 1427 | OH | TYR | 180 | 19.755 | 40.931 | 82.789 | 1.00 | 16.65 | DIC |
| ATOM | 1428 | C | TYR | 180 | 16.215 | 39.824 | 75.831 | 1.00 | 19.02 | DIC |
| ATOM | 1429 | O | TYR | 180 | 16.109 | 40.972 | 75.409 | 1.00 | 18.33 | DIC |
| ATOM | 1430 | N | LYS | 181 | 15.837 | 38.760 | 75.132 | 1.00 | 19.32 | DIC |
| ATOM | 1431 | CA | LYS | 181 | 15.291 | 38.879 | 73.785 | 1.00 | 20.45 | DIC |
| ATOM | 1432 | CB | LYS | 181 | 14.848 | 37.496 | 73.299 | 1.00 | 21.34 | DIC |
| ATOM | 1433 | CG | LYS | 181 | 14.196 | 37.475 | 71.922 | 1.00 | 24.95 | DIC |
| ATOM | 1434 | CD | LYS | 181 | 15.149 | 37.926 | 70.818 | 1.00 | 26.37 | DIC |
| ATOM | 1435 | CE | LYS | 181 | 14.466 | 37.879 | 69.456 | 1.00 | 27.47 | DIC |
| ATOM | 1436 | NZ | LYS | 181 | 14.072 | 36.492 | 69.067 | 1.00 | 29.43 | DIC |
| ATOM | 1437 | C | LYS | 181 | 14.133 | 39.870 | 73.619 | 1.00 | 20.56 | DIC |
| ATOM | 1438 | O | LYS | 181 | 14.045 | 40.560 | 72.608 | 1.00 | 21.00 | DIC |
| ATOM | 1439 | N | ASP | 182 | 13.253 | 39.947 | 74.610 | 1.00 | 20.10 | DIC |
| ATOM | 1440 | CA | ASP | 182 | 12.088 | 40.823 | 74.519 | 1.00 | 20.96 | DIC |
| ATOM | 1441 | CB | ASP | 182 | 10.964 | 40.275 | 75.402 | 1.00 | 21.98 | DIC |
| ATOM | 1442 | CG | ASP | 182 | 10.374 | 38.968 | 74.873 | 1.00 | 23.22 | DIC |
| ATOM | 1443 | OD1 | ASP | 182 | 9.574 | 38.346 | 75.602 | 1.00 | 24.34 | DIC |
| ATOM | 1444 | OD2 | ASP | 182 | 10.691 | 38.560 | 73.734 | 1.00 | 24.33 | DIC |
| ATOM | 1445 | C | ASP | 182 | 12.334 | 42.288 | 74.881 | 1.00 | 20.33 | DIC |
| ATOM | 1446 | O | ASP | 182 | 11.398 | 43.086 | 74.906 | 1.00 | 20.87 | DIC |
| ATOM | 1447 | N | VAL | 183 | 13.580 | 42.652 | 75.151 | 1.00 | 18.31 | DIC |
| ATOM | 1448 | CA | VAL | 183 | 13.866 | 44.023 | 75.532 | 1.00 | 17.11 | DIC |
| ATOM | 1449 | CB | VAL | 183 | 14.052 | 44.116 | 77.064 | 1.00 | 17.36 | DIC |
| ATOM | 1450 | CG1 | VAL | 183 | 15.351 | 43.427 | 77.482 | 1.00 | 15.90 | DIC |
| ATOM | 1451 | CG2 | VAL | 183 | 14.031 | 45.558 | 77.501 | 1.00 | 18.60 | DIC |
| ATOM | 1452 | C | VAL | 183 | 15.091 | 44.619 | 74.843 | 1.00 | 16.46 | DIC |
| ATOM | 1453 | O | VAL | 183 | 15.213 | 45.836 | 74.743 | 1.00 | 15.97 | DIC |
| ATOM | 1454 | N | MSE | 184 | 16.002 | 43.775 | 74.370 | 1.00 | 15.66 | DIC |
| ATOM | 1455 | CA | MSE | 184 | 17.195 | 44.292 | 73.714 | 1.00 | 14.63 | DIC |
| ATOM | 1456 | CB | MSE | 184 | 18.193 | 43.153 | 73.422 | 1.00 | 15.84 | DIC |
| ATOM | 1457 | CG | MSE | 184 | 17.673 | 41.995 | 72.601 | 1.00 | 15.47 | DIC |
| ATOM | 1458 | SE | MSE | 184 | 18.875 | 40.472 | 72.749 | 1.00 | 16.06 | DIC |
| ATOM | 1459 | CE | MSE | 184 | 20.077 | 40.875 | 71.295 | 1.00 | 14.11 | DIC |
| ATOM | 1460 | C | MSE | 184 | 16.816 | 45.053 | 72.448 | 1.00 | 14.45 | DIC |
| ATOM | 1461 | O | MSE | 184 | 15.944 | 44.623 | 71.683 | 1.00 | 13.76 | DIC |
| ATOM | 1462 | N | GLN | 185 | 17.456 | 46.203 | 72.248 | 1.00 | 12.74 | DIC |
| ATOM | 1463 | CA | GLN | 185 | 17.183 | 47.050 | 71.094 | 1.00 | 11.64 | DIC |
| ATOM | 1464 | CB | GLN | 185 | 16.728 | 48.438 | 71.565 | 1.00 | 13.80 | DIC |
| ATOM | 1465 | CG | GLN | 185 | 15.491 | 48.436 | 72.469 | 1.00 | 12.21 | DIC |
| ATOM | 1466 | CD | GLN | 185 | 15.125 | 49.825 | 72.981 | 1.00 | 13.45 | DIC |
| ATOM | 1467 | OE1 | GLN | 185 | 15.701 | 50.831 | 72.565 | 1.00 | 12.43 | DIC |
| ATOM | 1468 | NE2 | GLN | 185 | 14.159 | 49.881 | 73.888 | 1.00 | 12.29 | DIC |
| ATOM | 1469 | C | GLN | 185 | 18.406 | 47.211 | 70.198 | 1.00 | 12.26 | DIC |
| ATOM | 1470 | O | GLN | 185 | 18.299 | 47.714 | 69.072 | 1.00 | 10.28 | DIC |
| ATOM | 1471 | N | TYR | 186 | 19.571 | 46.784 | 70.680 | 1.00 | 10.35 | DIC |
| ATOM | 1472 | CA | TYR | 186 | 20.773 | 46.965 | 69.884 | 1.00 | 11.71 | DIC |
| ATOM | 1473 | CB | TYR | 186 | 21.709 | 47.965 | 70.579 | 1.00 | 11.81 | DIC |
| ATOM | 1474 | CG | TYR | 186 | 21.052 | 49.319 | 70.763 | 1.00 | 13.33 | DIC |
| ATOM | 1475 | CD1 | TYR | 186 | 20.566 | 49.720 | 72.007 | 1.00 | 13.33 | DIC |
| ATOM | 1476 | CE1 | TYR | 186 | 19.883 | 50.927 | 72.160 | 1.00 | 13.45 | DIC |
| ATOM | 1477 | CD2 | TYR | 186 | 20.844 | 50.165 | 69.669 | 1.00 | 14.56 | DIC |
| ATOM | 1478 | CE2 | TYR | 186 | 20.161 | 51.371 | 69.809 | 1.00 | 14.44 | DIC |
| ATOM | 1479 | CZ | TYR | 186 | 19.683 | 51.745 | 71.059 | 1.00 | 14.43 | DIC |
| ATOM | 1480 | OH | TYR | 186 | 19.010 | 52.937 | 71.204 | 1.00 | 12.04 | DIC |
| ATOM | 1481 | C | TYR | 186 | 21.535 | 45.725 | 69.441 | 1.00 | 10.15 | DIC |
| ATOM | 1482 | O | TYR | 186 | 22.730 | 45.791 | 69.167 | 1.00 | 10.75 | DIC |
| ATOM | 1483 | N | GLN | 187 | 20.829 | 44.603 | 69.376 | 1.00 | 8.94 | DIC |
| ATOM | 1484 | CA | GLN | 187 | 21.379 | 43.349 | 68.878 | 1.00 | 8.88 | DIC |
| ATOM | 1485 | CB | GLN | 187 | 21.399 | 43.447 | 67.342 | 1.00 | 9.31 | DIC |
| ATOM | 1486 | CG | GLN | 187 | 19.987 | 43.785 | 66.826 | 1.00 | 10.51 | DIC |
| ATOM | 1487 | CD | GLN | 187 | 19.891 | 44.176 | 65.360 | 1.00 | 10.67 | DIC |
| ATOM | 1488 | OE1 | GLN | 187 | 18.788 | 44.374 | 64.849 | 1.00 | 12.93 | DIC |
| ATOM | 1489 | NE2 | GLN | 187 | 21.021 | 44.303 | 64.684 | 1.00 | 10.73 | DIC |
| ATOM | 1490 | C | GLN | 187 | 22.726 | 42.898 | 69.458 | 1.00 | 8.99 | DIC |
| ATOM | 1491 | O | GLN | 187 | 22.833 | 42.742 | 70.671 | 1.00 | 9.41 | DIC |
| ATOM | 1492 | N | ASP | 188 | 23.742 | 42.675 | 68.618 | 1.00 | 8.46 | DIC |
| ATOM | 1493 | CA | ASP | 188 | 25.040 | 42.207 | 69.112 | 1.00 | 9.37 | DIC |
| ATOM | 1494 | CB | ASP | 188 | 26.017 | 41.980 | 67.951 | 1.00 | 10.94 | DIC |
| ATOM | 1495 | CG | ASP | 188 | 26.258 | 43.231 | 67.147 | 1.00 | 11.67 | DIC |
| ATOM | 1496 | OD1 | ASP | 188 | 25.359 | 43.619 | 66.370 | 1.00 | 12.99 | DIC |
| ATOM | 1497 | OD2 | ASP | 188 | 27.340 | 43.832 | 67.298 | 1.00 | 12.63 | DIC |
| ATOM | 1498 | C | ASP | 188 | 25.692 | 43.140 | 70.131 | 1.00 | 9.34 | DIC |
| ATOM | 1499 | O | ASP | 188 | 26.444 | 42.707 | 71.006 | 1.00 | 8.86 | DIC |
| ATOM | 1500 | N | GLN | 189 | 25.393 | 44.422 | 70.020 | 1.00 | 8.62 | DIC |
| ATOM | 1501 | CA | GLN | 189 | 25.955 | 45.401 | 70.928 | 1.00 | 9.00 | DIC |
| ATOM | 1502 | CB | GLN | 189 | 25.620 | 46.803 | 70.419 | 1.00 | 8.38 | DIC |
| ATOM | 1503 | CG | GLN | 189 | 26.246 | 47.941 | 71.193 | 1.00 | 8.88 | DIC |
| ATOM | 1504 | CD | GLN | 189 | 26.110 | 49.255 | 70.445 | 1.00 | 9.01 | DIC |
| ATOM | 1505 | OE1 | GLN | 189 | 27.025 | 49.673 | 69.730 | 1.00 | 10.27 | DIC |
| ATOM | 1506 | NE2 | GLN | 189 | 24.953 | 49.898 | 70.583 | 1.00 | 7.31 | DIC |
| ATOM | 1507 | C | GLN | 189 | 25.406 | 45.165 | 72.337 | 1.00 | 9.05 | DIC |
| ATOM | 1508 | O | GLN | 189 | 26.139 | 45.269 | 73.318 | 1.00 | 8.94 | DIC |
| ATOM | 1509 | N | ASP | 190 | 24.118 | 44.841 | 72.438 | 1.00 | 9.37 | DIC |
| ATOM | 1510 | CA | ASP | 190 | 23.527 | 44.569 | 73.739 | 1.00 | 9.91 | DIC |
| ATOM | 1511 | CB | ASP | 190 | 22.029 | 44.301 | 73.623 | 1.00 | 9.65 | DIC |
| ATOM | 1512 | CG | ASP | 190 | 21.222 | 45.563 | 73.412 | 1.00 | 10.87 | DIC |
| ATOM | 1513 | OD1 | ASP | 190 | 20.179 | 45.475 | 72.740 | 1.00 | 8.70 | DIC |
| ATOM | 1514 | OD2 | ASP | 190 | 21.618 | 46.632 | 73.928 | 1.00 | 10.04 | DIC |
| ATOM | 1515 | C | ASP | 190 | 24.196 | 43.346 | 74.341 | 1.00 | 9.18 | DIC |
| ATOM | 1516 | O | ASP | 190 | 24.581 | 43.365 | 75.497 | 1.00 | 10.40 | DIC |
| ATOM | 1517 | N | ILE | 191 | 24.331 | 42.284 | 73.554 | 1.00 | 9.00 | DIC |
| ATOM | 1518 | CA | ILE | 191 | 24.951 | 41.056 | 74.042 | 1.00 | 8.87 | DIC |
| ATOM | 1519 | CB | ILE | 191 | 25.086 | 40.023 | 72.917 | 1.00 | 8.34 | DIC |
| ATOM | 1520 | CG2 | ILE | 191 | 25.767 | 38.761 | 73.448 | 1.00 | 7.88 | DIC |
| ATOM | 1521 | CG1 | ILE | 191 | 23.699 | 39.689 | 72.363 | 1.00 | 8.24 | DIC |
| ATOM | 1522 | CD | ILE | 191 | 23.732 | 38.844 | 71.104 | 1.00 | 7.49 | DIC |
| ATOM | 1523 | C | ILE | 191 | 26.327 | 41.283 | 74.662 | 1.00 | 9.65 | DIC |
| ATOM | 1524 | O | ILE | 191 | 26.589 | 40.839 | 75.783 | 1.00 | 9.14 | DIC |
| ATOM | 1525 | N | LEU | 192 | 27.201 | 41.979 | 73.936 | 1.00 | 9.83 | DIC |
| ATOM | 1526 | CA | LEU | 192 | 28.550 | 42.260 | 74.413 | 1.00 | 9.59 | DIC |
| ATOM | 1527 | CB | LEU | 192 | 29.331 | 43.055 | 73.362 | 1.00 | 9.02 | DIC |
| ATOM | 1528 | CG | LEU | 192 | 29.555 | 42.376 | 72.012 | 1.00 | 8.81 | DIC |
| ATOM | 1529 | CD1 | LEU | 192 | 30.078 | 43.409 | 71.009 | 1.00 | 7.76 | DIC |
| ATOM | 1530 | CD2 | LEU | 192 | 30.530 | 41.225 | 72.167 | 1.00 | 9.35 | DIC |
| ATOM | 1531 | C | LEU | 192 | 28.538 | 43.030 | 75.733 | 1.00 | 9.85 | DIC |
| ATOM | 1532 | O | LEU | 192 | 29.276 | 42.695 | 76.656 | 1.00 | 9.13 | DIC |
| ATOM | 1533 | N | ASN | 193 | 27.706 | 44.062 | 75.823 | 1.00 | 9.48 | DIC |
| ATOM | 1534 | CA | ASN | 193 | 27.612 | 44.850 | 77.052 | 1.00 | 9.51 | DIC |
| ATOM | 1535 | CB | ASN | 193 | 26.676 | 46.045 | 76.849 | 1.00 | 9.70 | DIC |
| ATOM | 1536 | CG | ASN | 193 | 27.394 | 47.256 | 76.290 | 1.00 | 10.74 | DIC |
| ATOM | 1537 | OD1 | ASN | 193 | 28.041 | 47.996 | 77.033 | 1.00 | 8.96 | DIC |
| ATOM | 1538 | ND2 | ASN | 193 | 27.297 | 47.461 | 74.972 | 1.00 | 10.60 | DIC |
| ATOM | 1539 | C | ASN | 193 | 27.102 | 44.011 | 78.221 | 1.00 | 9.96 | DIC |
| ATOM | 1540 | O | ASN | 193 | 27.620 | 44.109 | 79.334 | 1.00 | 9.61 | DIC |
| ATOM | 1541 | N | GLY | 194 | 26.079 | 43.199 | 77.963 | 1.00 | 10.54 | DIC |
| ATOM | 1542 | CA | GLY | 194 | 25.513 | 42.369 | 79.011 | 1.00 | 11.12 | DIC |
| ATOM | 1543 | C | GLY | 194 | 26.479 | 41.326 | 79.532 | 1.00 | 11.80 | DIC |
| ATOM | 1544 | O | GLY | 194 | 26.612 | 41.139 | 80.746 | 1.00 | 11.37 | DIC |
| ATOM | 1545 | N | LEU | 195 | 27.162 | 40.643 | 78.618 | 1.00 | 11.19 | DIC |
| ATOM | 1546 | CA | LEU | 195 | 28.111 | 39.605 | 79.004 | 1.00 | 10.80 | DIC |
| ATOM | 1547 | CB | LEU | 195 | 28.557 | 38.788 | 77.785 | 1.00 | 7.99 | DIC |
| ATOM | 1548 | CG | LEU | 195 | 27.589 | 37.864 | 77.048 | 1.00 | 7.39 | DIC |
| ATOM | 1549 | CD1 | LEU | 195 | 28.334 | 37.170 | 75.910 | 1.00 | 6.05 | DIC |
| ATOM | 1550 | CD2 | LEU | 195 | 27.021 | 36.821 | 78.012 | 1.00 | 6.98 | DIC |
| ATOM | 1551 | C | LEU | 195 | 29.370 | 40.111 | 79.702 | 1.00 | 11.37 | DIC |
| ATOM | 1552 | O | LEU | 195 | 29.864 | 39.464 | 80.623 | 1.00 | 11.30 | DIC |
| ATOM | 1553 | N | PHE | 196 | 29.889 | 41.259 | 79.271 | 1.00 | 11.32 | DIC |
| ATOM | 1554 | CA | PHE | 196 | 31.143 | 41.763 | 79.831 | 1.00 | 11.00 | DIC |
| ATOM | 1555 | CB | PHE | 196 | 32.106 | 42.123 | 78.690 | 1.00 | 10.77 | DIC |
| ATOM | 1556 | CG | PHE | 196 | 32.297 | 41.017 | 77.689 | 1.00 | 11.74 | DIC |
| ATOM | 1557 | CD1 | PHE | 196 | 32.353 | 39.690 | 78.102 | 1.00 | 10.22 | DIC |
| ATOM | 1558 | CD2 | PHE | 196 | 32.445 | 41.302 | 76.339 | 1.00 | 10.79 | DIC |
| ATOM | 1559 | CE1 | PHE | 196 | 32.560 | 38.658 | 77.184 | 1.00 | 11.95 | DIC |
| ATOM | 1560 | CE2 | PHE | 196 | 32.649 | 40.278 | 75.411 | 1.00 | 11.65 | DIC |
| ATOM | 1561 | CZ | PHE | 196 | 32.708 | 38.955 | 75.836 | 1.00 | 11.34 | DIC |
| ATOM | 1562 | C | PHE | 196 | 31.103 | 42.923 | 80.812 | 1.00 | 11.21 | DIC |
| ATOM | 1563 | O | PHE | 196 | 32.154 | 43.356 | 81.280 | 1.00 | 10.49 | DIC |
| ATOM | 1564 | N | LYS | 197 | 29.912 | 43.428 | 81.119 | 1.00 | 11.58 | DIC |
| ATOM | 1565 | CA | LYS | 197 | 29.773 | 44.544 | 82.050 | 1.00 | 11.43 | DIC |
| ATOM | 1566 | CB | LYS | 197 | 28.291 | 44.737 | 82.417 | 1.00 | 11.39 | DIC |
| ATOM | 1567 | CG | LYS | 197 | 27.594 | 43.539 | 83.091 | 1.00 | 12.71 | DIC |
| ATOM | 1568 | CD | LYS | 197 | 26.097 | 43.828 | 83.262 | 1.00 | 11.95 | DIC |
| ATOM | 1569 | CE | LYS | 197 | 25.351 | 42.749 | 84.053 | 1.00 | 12.18 | DIC |
| ATOM | 1570 | NZ | LYS | 197 | 25.288 | 41.442 | 83.336 | 1.00 | 10.84 | DIC |
| ATOM | 1571 | C | LYS | 197 | 30.612 | 44.304 | 83.305 | 1.00 | 12.30 | DIC |
| ATOM | 1572 | O | LYS | 197 | 30.632 | 43.199 | 83.846 | 1.00 | 11.29 | DIC |
| ATOM | 1573 | N | GLY | 198 | 31.321 | 45.333 | 83.758 | 1.00 | 12.29 | DIC |
| ATOM | 1574 | CA | GLY | 198 | 32.153 | 45.182 | 84.942 | 1.00 | 13.17 | DIC |
| ATOM | 1575 | C | GLY | 198 | 33.546 | 44.680 | 84.614 | 1.00 | 13.23 | DIC |
| ATOM | 1576 | O | GLY | 198 | 34.450 | 44.733 | 85.453 | 1.00 | 12.63 | DIC |
| ATOM | 1577 | N | GLY | 199 | 33.718 | 44.184 | 83.390 | 1.00 | 13.28 | DIC |
| ATOM | 1578 | CA | GLY | 199 | 35.011 | 43.688 | 82.952 | 1.00 | 13.74 | DIC |
| ATOM | 1579 | C | GLY | 199 | 35.387 | 44.341 | 81.628 | 1.00 | 14.37 | DIC |
| ATOM | 1580 | O | GLY | 199 | 35.845 | 43.680 | 80.696 | 1.00 | 13.49 | DIC |
| ATOM | 1581 | N | VAL | 200 | 35.176 | 45.651 | 81.560 | 1.00 | 13.59 | DIC |
| ATOM | 1582 | CA | VAL | 200 | 35.468 | 46.446 | 80.374 | 1.00 | 14.48 | DIC |
| ATOM | 1583 | CB | VAL | 200 | 34.172 | 47.103 | 79.823 | 1.00 | 14.24 | DIC |
| ATOM | 1584 | CG1 | VAL | 200 | 34.506 | 48.063 | 78.700 | 1.00 | 16.87 | DIC |
| ATOM | 1585 | CG2 | VAL | 200 | 33.206 | 46.040 | 79.332 | 1.00 | 15.73 | DIC |
| ATOM | 1586 | C | VAL | 200 | 36.455 | 47.578 | 80.678 | 1.00 | 14.42 | DIC |
| ATOM | 1587 | O | VAL | 200 | 36.314 | 48.282 | 81.673 | 1.00 | 14.37 | DIC |
| ATOM | 1588 | N | CYS | 201 | 37.451 | 47.737 | 79.813 | 1.00 | 14.62 | DIC |
| ATOM | 1589 | CA | CYS | 201 | 38.426 | 48.814 | 79.935 | 1.00 | 15.36 | DIC |
| ATOM | 1590 | CB | CYS | 201 | 39.797 | 48.346 | 79.445 | 1.00 | 17.87 | DIC |
| ATOM | 1591 | SG | CYS | 201 | 41.042 | 49.652 | 79.343 | 1.00 | 24.36 | DIC |
| ATOM | 1592 | C | CYS | 201 | 37.877 | 49.890 | 79.005 | 1.00 | 14.95 | DIC |
| ATOM | 1593 | O | CYS | 201 | 37.561 | 49.599 | 77.856 | 1.00 | 14.31 | DIC |
| ATOM | 1594 | N | TYR | 202 | 37.729 | 51.122 | 79.479 | 1.00 | 14.76 | DIC |
| ATOM | 1595 | CA | TYR | 202 | 37.184 | 52.146 | 78.598 | 1.00 | 14.81 | DIC |
| ATOM | 1596 | CB | TYR | 202 | 36.228 | 53.076 | 79.346 | 1.00 | 16.13 | DIC |
| ATOM | 1597 | CG | TYR | 202 | 35.108 | 52.377 | 80.083 | 1.00 | 16.70 | DIC |
| ATOM | 1598 | CD1 | TYR | 202 | 35.199 | 52.148 | 81.455 | 1.00 | 16.70 | DIC |
| ATOM | 1599 | CE1 | TYR | 202 | 34.166 | 51.540 | 82.156 | 1.00 | 17.89 | DIC |
| ATOM | 1600 | CD2 | TYR | 202 | 33.948 | 51.967 | 79.418 | 1.00 | 17.16 | DIC |
| ATOM | 1601 | CE2 | TYR | 202 | 32.900 | 51.347 | 80.113 | 1.00 | 17.82 | DIC |
| ATOM | 1602 | CZ | TYR | 202 | 33.020 | 51.143 | 81.486 | 1.00 | 18.36 | DIC |
| ATOM | 1603 | OH | TYR | 202 | 31.997 | 50.566 | 82.208 | 1.00 | 18.52 | DIC |
| ATOM | 1604 | C | TYR | 202 | 38.264 | 52.974 | 77.923 | 1.00 | 14.97 | DIC |
| ATOM | 1605 | O | TYR | 202 | 39.193 | 53.447 | 78.571 | 1.00 | 15.35 | DIC |
| ATOM | 1606 | N | ALA | 203 | 38.128 | 53.132 | 76.609 | 1.00 | 13.97 | DIC |
| ATOM | 1607 | CA | ALA | 203 | 39.068 | 53.902 | 75.801 | 1.00 | 13.45 | DIC |
| ATOM | 1608 | CB | ALA | 203 | 39.284 | 53.210 | 74.463 | 1.00 | 14.46 | DIC |
| ATOM | 1609 | C | ALA | 203 | 38.490 | 55.293 | 75.579 | 1.00 | 12.66 | DIC |
| ATOM | 1610 | O | ALA | 203 | 37.290 | 55.495 | 75.748 | 1.00 | 12.27 | DIC |
| ATOM | 1611 | N | ASN | 204 | 39.331 | 56.252 | 75.201 | 1.00 | 11.38 | DIC |
| ATOM | 1612 | CA | ASN | 204 | 38.856 | 57.612 | 74.964 | 1.00 | 10.72 | DIC |
| ATOM | 1613 | CB | ASN | 204 | 40.036 | 58.556 | 74.754 | 1.00 | 11.13 | DIC |
| ATOM | 1614 | CG | ASN | 204 | 39.620 | 59.996 | 74.762 | 1.00 | 10.71 | DIC |
| ATOM | 1615 | OD1 | ASN | 204 | 39.380 | 60.578 | 75.827 | 1.00 | 12.74 | DIC |
| ATOM | 1616 | ND2 | ASN | 204 | 39.503 | 60.584 | 73.578 | 1.00 | 10.78 | DIC |
| ATOM | 1617 | C | ASN | 204 | 37.945 | 57.645 | 73.734 | 1.00 | 10.83 | DIC |
| ATOM | 1618 | O | ASN | 204 | 38.100 | 56.833 | 72.815 | 1.00 | 9.54 | DIC |
| ATOM | 1619 | N | SER | 205 | 37.006 | 58.587 | 73.701 | 1.00 | 9.57 | DIC |
| ATOM | 1620 | CA | SER | 205 | 36.088 | 58.668 | 72.569 | 1.00 | 10.79 | DIC |
| ATOM | 1621 | CB | SER | 205 | 34.931 | 59.621 | 72.891 | 1.00 | 10.23 | DIC |
| ATOM | 1622 | OG | SER | 205 | 34.046 | 59.008 | 73.826 | 1.00 | 7.16 | DIC |
| ATOM | 1623 | C | SER | 205 | 36.740 | 59.052 | 71.251 | 1.00 | 10.81 | DIC |
| ATOM | 1624 | O | SER | 205 | 36.094 | 59.018 | 70.197 | 1.00 | 11.42 | DIC |
| ATOM | 1625 | N | ARG | 206 | 38.022 | 59.400 | 71.293 | 1.00 | 10.42 | DIC |
| ATOM | 1626 | CA | ARG | 206 | 38.728 | 59.759 | 70.064 | 1.00 | 10.38 | DIC |
| ATOM | 1627 | CB | ARG | 206 | 40.122 | 60.324 | 70.392 | 1.00 | 10.54 | DIC |
| ATOM | 1628 | CG | ARG | 206 | 41.103 | 59.329 | 70.992 | 1.00 | 11.01 | DIC |
| ATOM | 1629 | CD | ARG | 206 | 42.438 | 60.012 | 71.352 | 1.00 | 10.35 | DIC |
| ATOM | 1630 | NE | ARG | 206 | 42.972 | 60.735 | 70.200 | 1.00 | 11.14 | DIC |
| ATOM | 1631 | CZ | ARG | 206 | 44.020 | 60.350 | 69.477 | 1.00 | 9.85 | DIC |
| ATOM | 1632 | NH1 | ARG | 206 | 44.684 | 59.240 | 69.783 | 1.00 | 9.03 | DIC |
| ATOM | 1633 | NH2 | ARG | 206 | 44.389 | 61.069 | 68.426 | 1.00 | 7.77 | DIC |
| ATOM | 1634 | C | ARG | 206 | 38.854 | 58.508 | 69.188 | 1.00 | 9.72 | DIC |
| ATOM | 1635 | O | ARG | 206 | 39.059 | 58.593 | 67.973 | 1.00 | 8.91 | DIC |
| ATOM | 1636 | N | PHE | 207 | 38.719 | 57.341 | 69.812 | 1.00 | 9.74 | DIC |
| ATOM | 1637 | CA | PHE | 207 | 38.834 | 56.079 | 69.090 | 1.00 | 10.15 | DIC |
| ATOM | 1638 | CB | PHE | 207 | 39.583 | 55.053 | 69.947 | 1.00 | 10.78 | DIC |
| ATOM | 1639 | CG | PHE | 207 | 41.017 | 55.423 | 70.194 | 1.00 | 10.31 | DIC |
| ATOM | 1640 | CD1 | PHE | 207 | 41.452 | 55.792 | 71.465 | 1.00 | 10.66 | DIC |
| ATOM | 1641 | CD2 | PHE | 207 | 41.923 | 55.472 | 69.137 | 1.00 | 10.73 | DIC |
| ATOM | 1642 | CE1 | PHE | 207 | 42.771 | 56.215 | 71.674 | 1.00 | 10.88 | DIC |
| ATOM | 1643 | CE2 | PHE | 207 | 43.234 | 55.888 | 69.331 | 1.00 | 10.86 | DIC |
| ATOM | 1644 | CZ | PHE | 207 | 43.661 | 56.263 | 70.605 | 1.00 | 11.25 | DIC |
| ATOM | 1645 | C | PHE | 207 | 37.494 | 55.518 | 68.624 | 1.00 | 9.68 | DIC |
| ATOM | 1646 | O | PHE | 207 | 37.411 | 54.387 | 68.144 | 1.00 | 10.10 | DIC |
| ATOM | 1647 | N | ASN | 208 | 36.445 | 56.315 | 68.783 | 1.00 | 8.88 | DIC |
| ATOM | 1648 | CA | ASN | 208 | 35.114 | 55.936 | 68.335 | 1.00 | 9.98 | DIC |
| ATOM | 1649 | CB | ASN | 208 | 34.513 | 54.832 | 69.205 | 1.00 | 8.57 | DIC |
| ATOM | 1650 | CG | ASN | 208 | 33.371 | 54.116 | 68.507 | 1.00 | 7.87 | DIC |
| ATOM | 1651 | OD1 | ASN | 208 | 32.827 | 54.625 | 67.530 | 1.00 | 7.69 | DIC |
| ATOM | 1652 | ND2 | ASN | 208 | 33.002 | 52.939 | 69.000 | 1.00 | 6.25 | DIC |
| ATOM | 1653 | C | ASN | 208 | 34.253 | 57.188 | 68.397 | 1.00 | 10.42 | DIC |
| ATOM | 1654 | O | ASN | 208 | 33.368 | 57.316 | 69.240 | 1.00 | 11.59 | DIC |
| ATOM | 1655 | N | PHE | 209 | 34.532 | 58.109 | 67.482 | 1.00 | 10.12 | DIC |
| ATOM | 1656 | CA | PHE | 209 | 33.841 | 59.385 | 67.405 | 1.00 | 10.68 | DIC |
| ATOM | 1657 | CB | PHE | 209 | 34.788 | 60.421 | 66.784 | 1.00 | 10.56 | DIC |
| ATOM | 1658 | CG | PHE | 209 | 34.392 | 61.853 | 67.036 | 1.00 | 10.98 | DIC |
| ATOM | 1659 | CD1 | PHE | 209 | 34.475 | 62.401 | 68.311 | 1.00 | 12.28 | DIC |
| ATOM | 1660 | CD2 | PHE | 209 | 33.969 | 62.662 | 65.990 | 1.00 | 12.06 | DIC |
| ATOM | 1661 | CE1 | PHE | 209 | 34.141 | 63.742 | 68.545 | 1.00 | 12.45 | DIC |
| ATOM | 1662 | CE2 | PHE | 209 | 33.633 | 63.996 | 66.206 | 1.00 | 11.37 | DIC |
| ATOM | 1663 | CZ | PHE | 209 | 33.720 | 64.540 | 67.488 | 1.00 | 12.90 | DIC |
| ATOM | 1664 | C | PHE | 209 | 32.596 | 59.212 | 66.540 | 1.00 | 10.70 | DIC |
| ATOM | 1665 | O | PHE | 209 | 32.687 | 59.137 | 65.323 | 1.00 | 10.09 | DIC |
| ATOM | 1666 | N | MSE | 210 | 31.433 | 59.156 | 67.174 | 1.00 | 11.63 | DIC |
| ATOM | 1667 | CA | MSE | 210 | 30.178 | 58.963 | 66.446 | 1.00 | 12.51 | DIC |
| ATOM | 1668 | CB | MSE | 210 | 29.380 | 57.850 | 67.136 | 1.00 | 13.04 | DIC |
| ATOM | 1669 | CG | MSE | 210 | 30.140 | 56.526 | 67.190 | 1.00 | 12.66 | DIC |
| ATOM | 1670 | SE | MSE | 210 | 29.418 | 55.246 | 68.442 | 1.00 | 12.85 | DIC |
| ATOM | 1671 | CE | MSE | 210 | 30.266 | 55.875 | 70.045 | 1.00 | 11.55 | DIC |
| ATOM | 1672 | C | MSE | 210 | 29.364 | 60.253 | 66.363 | 1.00 | 12.05 | DIC |
| ATOM | 1673 | O | MSE | 210 | 29.735 | 61.265 | 66.958 | 1.00 | 12.49 | DIC |
| ATOM | 1674 | N | PRO | 211 | 28.245 | 60.240 | 65.613 | 1.00 | 12.41 | DIC |
| ATOM | 1675 | CD | PRO | 211 | 27.699 | 59.141 | 64.800 | 1.00 | 11.47 | DIC |
| ATOM | 1676 | CA | PRO | 211 | 27.408 | 61.439 | 65.482 | 1.00 | 11.06 | DIC |
| ATOM | 1677 | CB | PRO | 211 | 26.163 | 60.907 | 64.784 | 1.00 | 11.44 | DIC |
| ATOM | 1678 | CG | PRO | 211 | 26.732 | 59.865 | 63.882 | 1.00 | 11.64 | DIC |
| ATOM | 1679 | C | PRO | 211 | 27.074 | 62.121 | 66.817 | 1.00 | 11.44 | DIC |
| ATOM | 1680 | O | PRO | 211 | 27.073 | 63.355 | 66.915 | 1.00 | 11.07 | DIC |
| ATOM | 1681 | N | THR | 212 | 26.792 | 61.328 | 67.844 | 1.00 | 9.80 | DIC |
| ATOM | 1682 | CA | THR | 212 | 26.457 | 61.907 | 69.140 | 1.00 | 10.60 | DIC |
| ATOM | 1683 | CB | THR | 212 | 25.852 | 60.838 | 70.097 | 1.00 | 10.93 | DIC |
| ATOM | 1684 | OG1 | THR | 212 | 24.707 | 60.240 | 69.476 | 1.00 | 10.74 | DIC |
| ATOM | 1685 | CG2 | THR | 212 | 25.379 | 61.488 | 71.403 | 1.00 | 11.50 | DIC |
| ATOM | 1686 | C | THR | 212 | 27.656 | 62.609 | 69.798 | 1.00 | 11.02 | DIC |
| ATOM | 1687 | O | THR | 212 | 27.480 | 63.613 | 70.499 | 1.00 | 10.65 | DIC |
| ATOM | 1688 | N | ASN | 213 | 28.869 | 62.100 | 69.574 | 1.00 | 10.18 | DIC |
| ATOM | 1689 | CA | ASN | 213 | 30.064 | 62.725 | 70.148 | 1.00 | 10.69 | DIC |
| ATOM | 1690 | CB | ASN | 213 | 31.316 | 61.878 | 69.884 | 1.00 | 9.50 | DIC |
| ATOM | 1691 | CG | ASN | 213 | 31.212 | 60.462 | 70.459 | 1.00 | 8.75 | DIC |
| ATOM | 1692 | OD1 | ASN | 213 | 31.624 | 60.196 | 71.595 | 1.00 | 9.99 | DIC |
| ATOM | 1693 | ND2 | ASN | 213 | 30.651 | 59.556 | 69.677 | 1.00 | 5.77 | DIC |
| ATOM | 1694 | C | ASN | 213 | 30.229 | 64.083 | 69.482 | 1.00 | 12.35 | DIC |
| ATOM | 1695 | O | ASN | 213 | 30.591 | 65.068 | 70.130 | 1.00 | 12.36 | DIC |
| ATOM | 1696 | N | TYR | 214 | 29.961 | 64.128 | 68.180 | 1.00 | 12.87 | DIC |
| ATOM | 1697 | CA | TYR | 214 | 30.075 | 65.365 | 67.429 | 1.00 | 15.87 | DIC |
| ATOM | 1698 | CB | TYR | 214 | 29.837 | 65.117 | 65.940 | 1.00 | 17.04 | DIC |
| ATOM | 1699 | CG | TYR | 214 | 29.964 | 66.370 | 65.106 | 1.00 | 19.24 | DIC |
| ATOM | 1700 | CD1 | TYR | 214 | 31.212 | 66.888 | 64.777 | 1.00 | 19.84 | DIC |
| ATOM | 1701 | CE1 | TYR | 214 | 31.335 | 68.067 | 64.043 | 1.00 | 21.17 | DIC |
| ATOM | 1702 | CD2 | TYR | 214 | 28.834 | 67.062 | 64.683 | 1.00 | 20.61 | DIC |
| ATOM | 1703 | CE2 | TYR | 214 | 28.943 | 68.243 | 63.954 | 1.00 | 22.48 | DIC |
| ATOM | 1704 | CZ | TYR | 214 | 30.196 | 68.740 | 63.637 | 1.00 | 22.15 | DIC |
| ATOM | 1705 | OH | TYR | 214 | 30.304 | 69.912 | 62.914 | 1.00 | 23.53 | DIC |
| ATOM | 1706 | C | TYR | 214 | 29.045 | 66.363 | 67.939 | 1.00 | 16.93 | DIC |
| ATOM | 1707 | O | TYR | 214 | 29.375 | 67.506 | 68.254 | 1.00 | 16.76 | DIC |
| ATOM | 1708 | N | ALA | 215 | 27.792 | 65.929 | 68.011 | 1.00 | 18.68 | DIC |
| ATOM | 1709 | CA | ALA | 215 | 26.725 | 66.800 | 68.489 | 1.00 | 20.37 | DIC |
| ATOM | 1710 | CB | ALA | 215 | 25.399 | 66.039 | 68.505 | 1.00 | 20.09 | DIC |
| ATOM | 1711 | C | ALA | 215 | 27.064 | 67.326 | 69.888 | 1.00 | 22.19 | DIC |
| ATOM | 1712 | O | ALA | 215 | 26.742 | 68.465 | 70.229 | 1.00 | 22.00 | DIC |
| ATOM | 1713 | N | ALA | 216 | 27.734 | 66.501 | 70.686 | 1.00 | 23.55 | DIC |
| ATOM | 1714 | CA | ALA | 216 | 28.111 | 66.894 | 72.040 | 1.00 | 26.87 | DIC |
| ATOM | 1715 | CB | ALA | 216 | 28.597 | 65.676 | 72.822 | 1.00 | 26.18 | DIC |
| ATOM | 1716 | C | ALA | 216 | 29.176 | 67.995 | 72.060 | 1.00 | 29.18 | DIC |
| ATOM | 1717 | O | ALA | 216 | 29.416 | 68.606 | 73.097 | 1.00 | 29.39 | DIC |
| ATOM | 1718 | N | MSE | 217 | 29.814 | 68.244 | 70.919 | 1.00 | 31.87 | DIC |
| ATOM | 1719 | CA | MSE | 217 | 30.840 | 69.284 | 70.822 | 1.00 | 34.52 | DIC |
| ATOM | 1720 | CB | MSE | 217 | 31.525 | 69.245 | 69.453 | 1.00 | 37.00 | DIC |
| ATOM | 1721 | CG | MSE | 217 | 32.334 | 67.995 | 69.149 | 1.00 | 40.08 | DIC |
| ATOM | 1722 | SE | MSE | 217 | 33.910 | 67.778 | 70.249 | 1.00 | 46.50 | DIC |
| ATOM | 1723 | CE | MSE | 217 | 34.937 | 69.288 | 69.631 | 1.00 | 45.06 | DIC |
| ATOM | 1724 | C | MSE | 217 | 30.203 | 70.658 | 71.002 | 1.00 | 35.07 | DIC |
| ATOM | 1725 | OT1 | MSE | 217 | 30.688 | 71.459 | 71.837 | 1.00 | 15.87 | DIC |
| ATOM | 1726 | OT2 | MSE | 217 | 29.223 | 70.927 | 70.278 | 1.00 | 15.87 | DIC |
| ATOM | 1727 | CB | PRO | 222 | 24.458 | 67.558 | 78.759 | 1.00 | 28.83 | DIC |
| ATOM | 1728 | CG | PRO | 222 | 25.198 | 67.311 | 77.451 | 1.00 | 28.89 | DIC |
| ATOM | 1729 | C | PRO | 222 | 26.376 | 67.968 | 80.306 | 1.00 | 28.22 | DIC |
| ATOM | 1730 | O | PRO | 222 | 27.322 | 68.622 | 80.771 | 1.00 | 29.45 | DIC |
| ATOM | 1731 | N | PRO | 222 | 25.993 | 69.355 | 78.294 | 1.00 | 28.60 | DIC |
| ATOM | 1732 | CD | PRO | 222 | 25.675 | 68.707 | 77.009 | 1.00 | 29.20 | DIC |
| ATOM | 1733 | CA | PRO | 222 | 25.342 | 68.618 | 79.398 | 1.00 | 28.68 | DIC |
| ATOM | 1734 | N | ALA | 223 | 26.184 | 66.671 | 80.525 | 1.00 | 28.20 | DIC |
| ATOM | 1735 | CA | ALA | 223 | 27.038 | 65.838 | 81.366 | 1.00 | 27.95 | DIC |
| ATOM | 1736 | CB | ALA | 223 | 27.638 | 66.659 | 82.509 | 1.00 | 27.92 | DIC |
| ATOM | 1737 | C | ALA | 223 | 26.116 | 64.764 | 81.925 | 1.00 | 27.33 | DIC |
| ATOM | 1738 | O | ALA | 223 | 25.370 | 65.009 | 82.871 | 1.00 | 27.98 | DIC |
| ATOM | 1739 | N | THR | 224 | 26.158 | 63.578 | 81.335 | 1.00 | 27.53 | DIC |
| ATOM | 1740 | CA | THR | 224 | 25.294 | 62.494 | 81.787 | 1.00 | 27.74 | DIC |
| ATOM | 1741 | CB | THR | 224 | 25.194 | 61.402 | 80.692 | 1.00 | 27.45 | DIC |
| ATOM | 1742 | OG1 | THR | 224 | 25.090 | 60.106 | 81.294 | 1.00 | 28.28 | DIC |
| ATOM | 1743 | CG2 | THR | 224 | 26.408 | 61.470 | 79.761 | 1.00 | 29.04 | DIC |
| ATOM | 1744 | C | THR | 224 | 25.700 | 61.883 | 83.136 | 1.00 | 27.43 | DIC |
| ATOM | 1745 | O | THR | 224 | 24.856 | 61.344 | 83.856 | 1.00 | 28.04 | DIC |
| ATOM | 1746 | N | HIS | 225 | 26.981 | 61.983 | 83.480 | 1.00 | 26.82 | DIC |
| ATOM | 1747 | CA | HIS | 225 | 27.491 | 61.455 | 84.746 | 1.00 | 25.60 | DIC |
| ATOM | 1748 | CB | HIS | 225 | 28.100 | 60.070 | 84.541 | 1.00 | 25.60 | DIC |
| ATOM | 1749 | CG | HIS | 225 | 27.095 | 59.000 | 84.263 | 1.00 | 25.36 | DIC |
| ATOM | 1750 | CD2 | HIS | 225 | 26.818 | 58.309 | 83.132 | 1.00 | 24.64 | DIC |
| ATOM | 1751 | ND1 | HIS | 225 | 26.231 | 58.525 | 85.225 | 1.00 | 25.09 | DIC |
| ATOM | 1752 | CE1 | HIS | 225 | 25.466 | 57.585 | 84.700 | 1.00 | 24.96 | DIC |
| ATOM | 1753 | NE2 | HIS | 225 | 25.801 | 57.435 | 83.431 | 1.00 | 25.41 | DIC |
| ATOM | 1754 | C | HIS | 225 | 28.565 | 62.391 | 85.289 | 1.00 | 25.02 | DIC |
| ATOM | 1755 | O | HIS | 225 | 29.326 | 62.967 | 84.516 | 1.00 | 25.07 | DIC |
| ATOM | 1756 | N | THR | 226 | 28.634 | 62.535 | 86.613 | 1.00 | 24.57 | DIC |
| ATOM | 1757 | CA | THR | 226 | 29.633 | 63.410 | 87.231 | 1.00 | 23.66 | DIC |
| ATOM | 1758 | CB | THR | 226 | 29.152 | 63.957 | 88.600 | 1.00 | 23.68 | DIC |
| ATOM | 1759 | OG1 | THR | 226 | 28.822 | 62.867 | 89.470 | 1.00 | 23.32 | DIC |
| ATOM | 1760 | CG2 | THR | 226 | 27.930 | 64.841 | 88.419 | 1.00 | 24.26 | DIC |
| ATOM | 1761 | C | THR | 226 | 30.958 | 62.676 | 87.430 | 1.00 | 22.94 | DIC |
| ATOM | 1762 | O | THR | 226 | 32.015 | 63.300 | 87.558 | 1.00 | 22.50 | DIC |
| ATOM | 1763 | N | ASP | 227 | 30.889 | 61.348 | 87.454 | 1.00 | 22.21 | DIC |
| ATOM | 1764 | CA | ASP | 227 | 32.071 | 60.509 | 87.616 | 1.00 | 21.20 | DIC |
| ATOM | 1765 | CB | ASP | 227 | 31.749 | 59.079 | 87.179 | 1.00 | 22.48 | DIC |
| ATOM | 1766 | CG | ASP | 227 | 32.969 | 58.177 | 87.182 | 1.00 | 22.78 | DIC |
| ATOM | 1767 | OD1 | ASP | 227 | 32.906 | 57.089 | 86.574 | 1.00 | 23.40 | DIC |
| ATOM | 1768 | OD2 | ASP | 227 | 33.988 | 58.551 | 87.795 | 1.00 | 23.59 | DIC |
| ATOM | 1769 | C | ASP | 227 | 33.226 | 61.044 | 86.767 | 1.00 | 21.18 | DIC |
| ATOM | 1770 | O | ASP | 227 | 33.110 | 61.149 | 85.551 | 1.00 | 20.24 | DIC |
| ATOM | 1771 | N | PRO | 228 | 34.358 | 61.381 | 87.401 | 1.00 | 20.52 | DIC |
| ATOM | 1772 | CD | PRO | 228 | 34.630 | 61.341 | 88.848 | 1.00 | 20.97 | DIC |
| ATOM | 1773 | CA | PRO | 228 | 35.514 | 61.901 | 86.662 | 1.00 | 19.86 | DIC |
| ATOM | 1774 | CB | PRO | 228 | 36.526 | 62.205 | 87.768 | 1.00 | 21.04 | DIC |
| ATOM | 1775 | CG | PRO | 228 | 36.130 | 61.267 | 88.878 | 1.00 | 21.72 | DIC |
| ATOM | 1776 | C | PRO | 228 | 36.061 | 60.959 | 85.589 | 1.00 | 19.43 | DIC |
| ATOM | 1777 | O | PRO | 228 | 36.535 | 61.411 | 84.548 | 1.00 | 18.48 | DIC |
| ATOM | 1778 | N | LEU | 229 | 35.996 | 59.653 | 85.827 | 1.00 | 18.78 | DIC |
| ATOM | 1779 | CA | LEU | 229 | 36.489 | 58.703 | 84.832 | 1.00 | 18.33 | DIC |
| ATOM | 1780 | CB | LEU | 229 | 36.450 | 57.279 | 85.382 | 1.00 | 18.37 | DIC |
| ATOM | 1781 | CG | LEU | 229 | 36.958 | 56.164 | 84.464 | 1.00 | 19.20 | DIC |
| ATOM | 1782 | CD1 | LEU | 229 | 37.498 | 55.030 | 85.319 | 1.00 | 19.93 | DIC |
| ATOM | 1783 | CD2 | LEU | 229 | 35.842 | 55.677 | 83.540 | 1.00 | 19.78 | DIC |
| ATOM | 1784 | C | LEU | 229 | 35.618 | 58.800 | 83.588 | 1.00 | 17.34 | DIC |
| ATOM | 1785 | O | LEU | 229 | 36.119 | 58.932 | 82.467 | 1.00 | 17.32 | DIC |
| ATOM | 1786 | N | TYR | 230 | 34.308 | 58.742 | 83.802 | 1.00 | 17.15 | DIC |
| ATOM | 1787 | CA | TYR | 230 | 33.335 | 58.832 | 82.724 | 1.00 | 15.74 | DIC |
| ATOM | 1788 | CB | TYR | 230 | 31.919 | 58.923 | 83.292 | 1.00 | 16.08 | DIC |
| ATOM | 1789 | CG | TYR | 230 | 30.850 | 58.982 | 82.225 | 1.00 | 14.76 | DIC |
| ATOM | 1790 | CD1 | TYR | 230 | 30.422 | 57.827 | 81.580 | 1.00 | 14.83 | DIC |
| ATOM | 1791 | CE1 | TYR | 230 | 29.469 | 57.877 | 80.569 | 1.00 | 14.30 | DIC |
| ATOM | 1792 | CD2 | TYR | 230 | 30.296 | 60.199 | 81.835 | 1.00 | 14.34 | DIC |
| ATOM | 1793 | CE2 | TYR | 230 | 29.345 | 60.261 | 80.827 | 1.00 | 15.23 | DIC |
| ATOM | 1794 | CZ | TYR | 230 | 28.935 | 59.093 | 80.196 | 1.00 | 14.70 | DIC |
| ATOM | 1795 | OH | TYR | 230 | 27.993 | 59.146 | 79.190 | 1.00 | 14.53 | DIC |
| ATOM | 1796 | C | TYR | 230 | 33.601 | 60.075 | 81.892 | 1.00 | 16.16 | DIC |
| ATOM | 1797 | O | TYR | 230 | 33.603 | 60.027 | 80.665 | 1.00 | 15.11 | DIC |
| ATOM | 1798 | N | ARG | 231 | 33.809 | 61.195 | 82.574 | 1.00 | 16.82 | DIC |
| ATOM | 1799 | CA | ARG | 231 | 34.068 | 62.460 | 81.896 | 1.00 | 17.72 | DIC |
| ATOM | 1800 | CB | ARG | 231 | 34.045 | 63.606 | 82.915 | 1.00 | 20.64 | DIC |
| ATOM | 1801 | CG | ARG | 231 | 32.640 | 63.909 | 83.459 | 1.00 | 24.03 | DIC |
| ATOM | 1802 | CD | ARG | 231 | 32.702 | 64.690 | 84.769 | 1.00 | 27.73 | DIC |
| ATOM | 1803 | NE | ARG | 231 | 33.599 | 65.839 | 84.672 | 1.00 | 31.57 | DIC |
| ATOM | 1804 | CZ | ARG | 231 | 33.335 | 66.937 | 83.971 | 1.00 | 32.63 | DIC |
| ATOM | 1805 | NH1 | ARG | 231 | 34.216 | 67.930 | 83.933 | 1.00 | 33.24 | DIC |
| ATOM | 1806 | NH2 | ARG | 231 | 32.182 | 67.048 | 83.321 | 1.00 | 33.69 | DIC |
| ATOM | 1807 | C | ARG | 231 | 35.394 | 62.429 | 81.143 | 1.00 | 17.46 | DIC |
| ATOM | 1808 | O | ARG | 231 | 35.499 | 62.956 | 80.036 | 1.00 | 16.04 | DIC |
| ATOM | 1809 | N | ASP | 232 | 36.402 | 61.798 | 81.736 | 1.00 | 16.62 | DIC |
| ATOM | 1810 | CA | ASP | 232 | 37.708 | 61.710 | 81.097 | 1.00 | 17.02 | DIC |
| ATOM | 1811 | CB | ASP | 232 | 38.698 | 61.039 | 82.055 | 1.00 | 16.63 | DIC |
| ATOM | 1812 | CG | ASP | 232 | 40.058 | 60.792 | 81.425 | 1.00 | 18.06 | DIC |
| ATOM | 1813 | OD1 | ASP | 232 | 40.216 | 59.786 | 80.698 | 1.00 | 18.50 | DIC |
| ATOM | 1814 | OD2 | ASP | 232 | 40.976 | 61.602 | 81.662 | 1.00 | 17.41 | DIC |
| ATOM | 1815 | C | ASP | 232 | 37.625 | 60.939 | 79.771 | 1.00 | 16.91 | DIC |
| ATOM | 1816 | O | ASP | 232 | 38.189 | 61.362 | 78.755 | 1.00 | 16.81 | DIC |
| ATOM | 1817 | N | ARG | 233 | 36.897 | 59.825 | 79.781 | 1.00 | 16.06 | DIC |
| ATOM | 1818 | CA | ARG | 233 | 36.748 | 58.985 | 78.598 | 1.00 | 14.55 | DIC |
| ATOM | 1819 | CB | ARG | 233 | 36.210 | 57.605 | 78.993 | 1.00 | 14.82 | DIC |
| ATOM | 1820 | CG | ARG | 233 | 37.033 | 56.881 | 80.036 | 1.00 | 13.79 | DIC |
| ATOM | 1821 | CD | ARG | 233 | 38.393 | 56.443 | 79.515 | 1.00 | 13.23 | DIC |
| ATOM | 1822 | NE | ARG | 233 | 39.008 | 55.517 | 80.460 | 1.00 | 13.51 | DIC |
| ATOM | 1823 | CZ | ARG | 233 | 39.560 | 55.875 | 81.618 | 1.00 | 14.87 | DIC |
| ATOM | 1824 | NH1 | ARG | 233 | 39.596 | 57.151 | 81.982 | 1.00 | 13.99 | DIC |
| ATOM | 1825 | NH2 | ARG | 233 | 40.042 | 54.947 | 82.435 | 1.00 | 13.15 | DIC |
| ATOM | 1826 | C | ARG | 233 | 35.830 | 59.572 | 77.533 | 1.00 | 14.38 | DIC |
| ATOM | 1827 | O | ARG | 233 | 36.076 | 59.396 | 76.341 | 1.00 | 14.52 | DIC |
| ATOM | 1828 | N | THR | 234 | 34.774 | 60.262 | 77.955 | 1.00 | 13.26 | DIC |
| ATOM | 1829 | CA | THR | 234 | 33.830 | 60.831 | 77.000 | 1.00 | 12.89 | DIC |
| ATOM | 1830 | CB | THR | 234 | 32.419 | 60.981 | 77.622 | 1.00 | 11.52 | DIC |
| ATOM | 1831 | OG1 | THR | 234 | 32.512 | 61.637 | 78.893 | 1.00 | 9.01 | DIC |
| ATOM | 1832 | CG2 | THR | 234 | 31.782 | 59.605 | 77.807 | 1.00 | 10.76 | DIC |
| ATOM | 1833 | C | THR | 234 | 34.283 | 62.153 | 76.399 | 1.00 | 13.61 | DIC |
| ATOM | 1834 | O | THR | 234 | 33.754 | 62.577 | 75.384 | 1.00 | 13.22 | DIC |
| ATOM | 1835 | N | ASN | 235 | 35.258 | 62.802 | 77.028 | 1.00 | 14.53 | DIC |
| ATOM | 1836 | CA | ASN | 235 | 35.802 | 64.048 | 76.501 | 1.00 | 15.83 | DIC |
| ATOM | 1837 | CB | ASN | 235 | 36.519 | 64.854 | 77.590 | 1.00 | 18.71 | DIC |
| ATOM | 1838 | CG | ASN | 235 | 37.384 | 65.976 | 77.009 | 1.00 | 20.95 | DIC |
| ATOM | 1839 | OD1 | ASN | 235 | 36.880 | 67.030 | 76.616 | 1.00 | 22.71 | DIC |
| ATOM | 1840 | ND2 | ASN | 235 | 38.693 | 65.739 | 76.935 | 1.00 | 23.01 | DIC |
| ATOM | 1841 | C | ASN | 235 | 36.822 | 63.618 | 75.457 | 1.00 | 15.52 | DIC |
| ATOM | 1842 | O | ASN | 235 | 37.901 | 63.125 | 75.794 | 1.00 | 13.95 | DIC |
| ATOM | 1843 | N | THR | 236 | 36.485 | 63.795 | 74.188 | 1.00 | 14.36 | DIC |
| ATOM | 1844 | CA | THR | 236 | 37.396 | 63.397 | 73.135 | 1.00 | 14.17 | DIC |
| ATOM | 1845 | CB | THR | 236 | 36.711 | 63.494 | 71.754 | 1.00 | 14.16 | DIC |
| ATOM | 1846 | OG1 | THR | 236 | 35.656 | 62.523 | 71.690 | 1.00 | 12.60 | DIC |
| ATOM | 1847 | CG2 | THR | 236 | 37.716 | 63.234 | 70.622 | 1.00 | 13.57 | DIC |
| ATOM | 1848 | C | THR | 236 | 38.667 | 64.228 | 73.142 | 1.00 | 14.15 | DIC |
| ATOM | 1849 | O | THR | 236 | 38.619 | 65.460 | 73.213 | 1.00 | 15.30 | DIC |
| ATOM | 1850 | N | VAL | 237 | 39.808 | 63.546 | 73.105 | 1.00 | 13.96 | DIC |
| ATOM | 1851 | CA | VAL | 237 | 41.094 | 64.227 | 73.070 | 1.00 | 14.29 | DIC |
| ATOM | 1852 | CB | VAL | 237 | 42.190 | 63.454 | 73.866 | 1.00 | 14.32 | DIC |
| ATOM | 1853 | CG1 | VAL | 237 | 43.530 | 64.173 | 73.749 | 1.00 | 14.22 | DIC |
| ATOM | 1854 | CG2 | VAL | 237 | 41.794 | 63.349 | 75.335 | 1.00 | 13.45 | DIC |
| ATOM | 1855 | C | VAL | 237 | 41.471 | 64.268 | 71.594 | 1.00 | 14.08 | DIC |
| ATOM | 1856 | O | VAL | 237 | 41.763 | 63.231 | 70.996 | 1.00 | 13.74 | DIC |
| ATOM | 1857 | N | MSE | 238 | 41.423 | 65.461 | 71.004 | 1.00 | 14.13 | DIC |
| ATOM | 1858 | CA | MSE | 238 | 41.753 | 65.636 | 69.593 | 1.00 | 14.57 | DIC |
| ATOM | 1859 | CB | MSE | 238 | 41.525 | 67.095 | 69.170 | 1.00 | 15.84 | DIC |
| ATOM | 1860 | CG | MSE | 238 | 40.078 | 67.574 | 69.271 | 1.00 | 18.43 | DIC |
| ATOM | 1861 | SE | MSE | 238 | 38.879 | 66.542 | 68.173 | 1.00 | 23.28 | DIC |
| ATOM | 1862 | CE | MSE | 238 | 37.208 | 66.992 | 69.030 | 1.00 | 22.22 | DIC |
| ATOM | 1863 | C | MSE | 238 | 43.205 | 65.251 | 69.313 | 1.00 | 13.92 | DIC |
| ATOM | 1864 | O | MSE | 238 | 44.050 | 65.297 | 70.208 | 1.00 | 13.64 | DIC |
| ATOM | 1865 | N | PRO | 239 | 43.508 | 64.845 | 68.066 | 1.00 | 13.35 | DIC |
| ATOM | 1866 | CD | PRO | 239 | 44.889 | 64.645 | 67.591 | 1.00 | 14.11 | DIC |
| ATOM | 1867 | CA | PRO | 239 | 42.566 | 64.743 | 66.949 | 1.00 | 13.23 | DIC |
| ATOM | 1868 | CB | PRO | 239 | 43.477 | 64.777 | 65.728 | 1.00 | 14.00 | DIC |
| ATOM | 1869 | CG | PRO | 239 | 44.675 | 64.036 | 66.220 | 1.00 | 14.44 | DIC |
| ATOM | 1870 | C | PRO | 239 | 41.761 | 63.452 | 67.009 | 1.00 | 13.04 | DIC |
| ATOM | 1871 | O | PRO | 239 | 42.125 | 62.512 | 67.721 | 1.00 | 12.63 | DIC |
| ATOM | 1872 | N | VAL | 240 | 40.668 | 63.417 | 66.253 | 1.00 | 12.39 | DIC |
| ATOM | 1873 | CA | VAL | 240 | 39.824 | 62.240 | 66.199 | 1.00 | 11.18 | DIC |
| ATOM | 1874 | CB | VAL | 240 | 38.567 | 62.506 | 65.352 | 1.00 | 11.67 | DIC |
| ATOM | 1875 | CG1 | VAL | 240 | 37.728 | 61.238 | 65.244 | 1.00 | 11.57 | DIC |
| ATOM | 1876 | CG2 | VAL | 240 | 37.755 | 63.641 | 65.980 | 1.00 | 11.98 | DIC |
| ATOM | 1877 | C | VAL | 240 | 40.660 | 61.150 | 65.550 | 1.00 | 10.08 | DIC |
| ATOM | 1878 | O | VAL | 240 | 41.329 | 61.403 | 64.553 | 1.00 | 9.26 | DIC |
| ATOM | 1879 | N | ALA | 241 | 40.645 | 59.951 | 66.125 | 1.00 | 8.61 | DIC |
| ATOM | 1880 | CA | ALA | 241 | 41.423 | 58.834 | 65.582 | 1.00 | 8.11 | DIC |
| ATOM | 1881 | CB | ALA | 241 | 42.072 | 58.040 | 66.716 | 1.00 | 8.10 | DIC |
| ATOM | 1882 | C | ALA | 241 | 40.545 | 57.916 | 64.739 | 1.00 | 7.76 | DIC |
| ATOM | 1883 | O | ALA | 241 | 41.000 | 57.341 | 63.749 | 1.00 | 7.78 | DIC |
| ATOM | 1884 | N | VAL | 242 | 39.282 | 57.788 | 65.133 | 1.00 | 7.63 | DIC |
| ATOM | 1885 | CA | VAL | 242 | 38.334 | 56.948 | 64.410 | 1.00 | 7.88 | DIC |
| ATOM | 1886 | CB | VAL | 242 | 38.101 | 55.605 | 65.152 | 1.00 | 6.91 | DIC |
| ATOM | 1887 | CG1 | VAL | 242 | 37.159 | 54.716 | 64.334 | 1.00 | 7.05 | DIC |
| ATOM | 1888 | CG2 | VAL | 242 | 39.435 | 54.894 | 65.400 | 1.00 | 5.82 | DIC |
| ATOM | 1889 | C | VAL | 242 | 36.965 | 57.599 | 64.228 | 1.00 | 9.17 | DIC |
| ATOM | 1890 | O | VAL | 242 | 36.296 | 57.908 | 65.207 | 1.00 | 9.42 | DIC |
| ATOM | 1891 | N | SER | 243 | 36.560 | 57.818 | 62.979 | 1.00 | 10.01 | DIC |
| ATOM | 1892 | CA | SER | 243 | 35.235 | 58.359 | 62.692 | 1.00 | 10.35 | DIC |
| ATOM | 1893 | CB | SER | 243 | 35.219 | 59.157 | 61.379 | 1.00 | 11.55 | DIC |
| ATOM | 1894 | OG | SER | 243 | 35.705 | 60.470 | 61.551 | 1.00 | 13.46 | DIC |
| ATOM | 1895 | C | SER | 243 | 34.356 | 57.133 | 62.530 | 1.00 | 10.90 | DIC |
| ATOM | 1896 | O | SER | 243 | 34.697 | 56.221 | 61.774 | 1.00 | 12.28 | DIC |
| ATOM | 1897 | N | HIS | 244 | 33.248 | 57.087 | 63.260 | 1.00 | 10.40 | DIC |
| ATOM | 1898 | CA | HIS | 244 | 32.335 | 55.955 | 63.157 | 1.00 | 9.48 | DIC |
| ATOM | 1899 | CB | HIS | 244 | 32.258 | 55.211 | 64.491 | 1.00 | 8.63 | DIC |
| ATOM | 1900 | CG | HIS | 244 | 31.406 | 53.969 | 64.457 | 1.00 | 7.07 | DIC |
| ATOM | 1901 | CD2 | HIS | 244 | 30.800 | 53.324 | 63.430 | 1.00 | 6.40 | DIC |
| ATOM | 1902 | ND1 | HIS | 244 | 31.109 | 53.242 | 65.593 | 1.00 | 5.32 | DIC |
| ATOM | 1903 | CE1 | HIS | 244 | 30.358 | 52.203 | 65.268 | 1.00 | 6.23 | DIC |
| ATOM | 1904 | NE2 | HIS | 244 | 30.155 | 52.228 | 63.961 | 1.00 | 5.08 | DIC |
| ATOM | 1905 | C | HIS | 244 | 30.962 | 56.518 | 62.778 | 1.00 | 9.32 | DIC |
| ATOM | 1906 | O | HIS | 244 | 30.357 | 57.262 | 63.518 | 1.00 | 9.29 | DIC |
| ATOM | 1907 | N | TYR | 245 | 30.491 | 56.106 | 61.614 | 1.00 | 10.23 | DIC |
| ATOM | 1908 | CA | TYR | 245 | 29.229 | 56.554 | 61.093 | 1.00 | 11.52 | DIC |
| ATOM | 1909 | CB | TYR | 245 | 29.385 | 56.723 | 59.580 | 1.00 | 12.42 | DIC |
| ATOM | 1910 | CG | TYR | 245 | 30.457 | 57.727 | 59.231 | 1.00 | 13.35 | DIC |
| ATOM | 1911 | CD1 | TYR | 245 | 30.201 | 59.088 | 59.354 | 1.00 | 14.28 | DIC |
| ATOM | 1912 | CE1 | TYR | 245 | 31.183 | 60.030 | 59.167 | 1.00 | 14.76 | DIC |
| ATOM | 1913 | CD2 | TYR | 245 | 31.750 | 57.323 | 58.888 | 1.00 | 13.50 | DIC |
| ATOM | 1914 | CE2 | TYR | 245 | 32.760 | 58.273 | 58.692 | 1.00 | 14.51 | DIC |
| ATOM | 1915 | CZ | TYR | 245 | 32.455 | 59.626 | 58.845 | 1.00 | 14.66 | DIC |
| ATOM | 1916 | OH | TYR | 245 | 33.392 | 60.623 | 58.735 | 1.00 | 16.02 | DIC |
| ATOM | 1917 | C | TYR | 245 | 28.023 | 55.657 | 61.442 | 1.00 | 12.60 | DIC |
| ATOM | 1918 | O | TYR | 245 | 27.217 | 55.324 | 60.565 | 1.00 | 10.16 | DIC |
| ATOM | 1919 | N | CYS | 246 | 27.868 | 55.278 | 62.716 | 1.00 | 14.89 | DIC |
| ATOM | 1920 | CA | CYS | 246 | 26.747 | 54.389 | 63.105 | 1.00 | 14.89 | DIC |
| ATOM | 1921 | CB | CYS | 246 | 26.837 | 54.053 | 64.586 | 1.00 | 14.89 | DIC |
| ATOM | 1922 | SG | CYS | 246 | 26.840 | 55.482 | 65.652 | 1.00 | 14.89 | DIC |
| ATOM | 1923 | C | CYS | 246 | 25.358 | 54.973 | 62.774 | 1.00 | 14.89 | DIC |
| ATOM | 1924 | O | CYS | 246 | 25.166 | 56.192 | 62.672 | 1.00 | 17.31 | DIC |
| ATOM | 1925 | N | GLY | 247 | 24.386 | 54.085 | 62.600 | 1.00 | 17.22 | DIC |
| ATOM | 1926 | CA | GLY | 247 | 23.050 | 54.509 | 62.224 | 1.00 | 15.49 | DIC |
| ATOM | 1927 | C | GLY | 247 | 22.836 | 54.344 | 60.719 | 1.00 | 15.79 | DIC |
| ATOM | 1928 | O | GLY | 247 | 23.737 | 53.891 | 60.024 | 1.00 | 14.94 | DIC |
| ATOM | 1929 | N | PRO | 248 | 21.638 | 54.668 | 60.197 | 1.00 | 15.38 | DIC |
| ATOM | 1930 | CD | PRO | 248 | 20.497 | 55.170 | 60.986 | 1.00 | 15.79 | DIC |
| ATOM | 1931 | CA | PRO | 248 | 21.275 | 54.566 | 58.776 | 1.00 | 15.64 | DIC |
| ATOM | 1932 | CB | PRO | 248 | 19.790 | 54.933 | 58.781 | 1.00 | 16.59 | DIC |
| ATOM | 1933 | CG | PRO | 248 | 19.691 | 55.890 | 59.926 | 1.00 | 16.76 | DIC |
| ATOM | 1934 | C | PRO | 248 | 22.078 | 55.449 | 57.812 | 1.00 | 15.27 | DIC |
| ATOM | 1935 | O | PRO | 248 | 22.254 | 55.102 | 56.643 | 1.00 | 14.87 | DIC |
| ATOM | 1936 | N | ALA | 249 | 22.558 | 56.590 | 58.301 | 1.00 | 15.42 | DIC |
| ATOM | 1937 | CA | ALA | 249 | 23.315 | 57.528 | 57.468 | 1.00 | 15.19 | DIC |
| ATOM | 1938 | CB | ALA | 249 | 23.165 | 58.938 | 58.029 | 1.00 | 15.27 | DIC |
| ATOM | 1939 | C | ALA | 249 | 24.793 | 57.173 | 57.328 | 1.00 | 15.38 | DIC |
| ATOM | 1940 | O | ALA | 249 | 25.592 | 57.446 | 58.220 | 1.00 | 15.05 | DIC |
| ATOM | 1941 | N | LYS | 250 | 25.152 | 56.584 | 56.188 | 1.00 | 14.72 | DIC |
| ATOM | 1942 | CA | LYS | 250 | 26.531 | 56.180 | 55.938 | 1.00 | 15.73 | DIC |
| ATOM | 1943 | CB | LYS | 250 | 26.566 | 54.754 | 55.382 | 1.00 | 13.72 | DIC |
| ATOM | 1944 | CG | LYS | 250 | 25.816 | 53.722 | 56.234 | 1.00 | 12.46 | DIC |
| ATOM | 1945 | CD | LYS | 250 | 26.365 | 53.637 | 57.655 | 1.00 | 11.65 | DIC |
| ATOM | 1946 | CE | LYS | 250 | 25.760 | 52.454 | 58.410 | 1.00 | 10.42 | DIC |
| ATOM | 1947 | NZ | LYS | 250 | 26.120 | 52.501 | 59.856 | 1.00 | 8.32 | DIC |
| ATOM | 1948 | C | LYS | 250 | 27.238 | 57.129 | 54.968 | 1.00 | 16.91 | DIC |
| ATOM | 1949 | O | LYS | 250 | 26.590 | 57.809 | 54.180 | 1.00 | 17.59 | DIC |
| ATOM | 1950 | N | PRO | 251 | 28.582 | 57.170 | 55.006 | 1.00 | 18.44 | DIC |
| ATOM | 1951 | CD | PRO | 251 | 29.488 | 56.380 | 55.861 | 1.00 | 18.27 | DIC |
| ATOM | 1952 | CA | PRO | 251 | 29.348 | 58.049 | 54.118 | 1.00 | 19.06 | DIC |
| ATOM | 1953 | CB | PRO | 251 | 30.767 | 57.936 | 54.663 | 1.00 | 18.51 | DIC |
| ATOM | 1954 | CG | PRO | 251 | 30.820 | 56.527 | 55.144 | 1.00 | 19.13 | DIC |
| ATOM | 1955 | C | PRO | 251 | 29.261 | 57.682 | 52.641 | 1.00 | 20.28 | DIC |
| ATOM | 1956 | O | PRO | 251 | 29.504 | 58.531 | 51.781 | 1.00 | 20.27 | DIC |
| ATOM | 1957 | N | TRP | 252 | 28.919 | 56.429 | 52.341 | 1.00 | 20.96 | DIC |
| ATOM | 1958 | CA | TRP | 252 | 28.799 | 55.997 | 50.945 | 1.00 | 21.97 | DIC |
| ATOM | 1959 | CB | TRP | 252 | 29.103 | 54.497 | 50.803 | 1.00 | 19.99 | DIC |
| ATOM | 1960 | CG | TRP | 252 | 28.408 | 53.601 | 51.785 | 1.00 | 17.82 | DIC |
| ATOM | 1961 | CD2 | TRP | 252 | 28.983 | 53.017 | 52.960 | 1.00 | 17.40 | DIC |
| ATOM | 1962 | CE2 | TRP | 252 | 27.976 | 52.253 | 53.581 | 1.00 | 16.50 | DIC |
| ATOM | 1963 | CE3 | TRP | 252 | 30.254 | 53.069 | 53.548 | 1.00 | 15.92 | DIC |
| ATOM | 1964 | CD1 | TRP | 252 | 27.113 | 53.180 | 51.743 | 1.00 | 17.54 | DIC |
| ATOM | 1965 | NE1 | TRP | 252 | 26.846 | 52.370 | 52.819 | 1.00 | 16.77 | DIC |
| ATOM | 1966 | CZ2 | TRP | 252 | 28.198 | 51.545 | 54.760 | 1.00 | 16.16 | DIC |
| ATOM | 1967 | CZ3 | TRP | 252 | 30.474 | 52.363 | 54.723 | 1.00 | 15.63 | DIC |
| ATOM | 1968 | CH2 | TRP | 252 | 29.451 | 51.612 | 55.316 | 1.00 | 16.00 | DIC |
| ATOM | 1969 | C | TRP | 252 | 27.420 | 56.320 | 50.375 | 1.00 | 23.39 | DIC |
| ATOM | 1970 | O | TRP | 252 | 27.060 | 55.873 | 49.284 | 1.00 | 23.70 | DIC |
| ATOM | 1971 | N | HIS | 253 | 26.666 | 57.117 | 51.125 | 1.00 | 25.29 | DIC |
| ATOM | 1972 | CA | HIS | 253 | 25.328 | 57.546 | 50.735 | 1.00 | 27.91 | DIC |
| ATOM | 1973 | CB | HIS | 253 | 24.389 | 57.480 | 51.941 | 1.00 | 28.69 | DIC |
| ATOM | 1974 | CG | HIS | 253 | 23.900 | 56.102 | 52.247 | 1.00 | 29.56 | DIC |
| ATOM | 1975 | CD2 | HIS | 253 | 23.892 | 54.973 | 51.499 | 1.00 | 29.18 | DIC |
| ATOM | 1976 | ND1 | HIS | 253 | 23.314 | 55.769 | 53.451 | 1.00 | 29.91 | DIC |
| ATOM | 1977 | CE1 | HIS | 253 | 22.969 | 54.494 | 53.431 | 1.00 | 30.98 | DIC |
| ATOM | 1978 | NE2 | HIS | 253 | 23.310 | 53.988 | 52.258 | 1.00 | 30.37 | DIC |
| ATOM | 1979 | C | HIS | 253 | 25.353 | 58.972 | 50.187 | 1.00 | 28.54 | DIC |
| ATOM | 1980 | OT1 | HIS | 253 | 26.453 | 59.565 | 50.159 | 1.00 | 15.87 | DIC |
| ATOM | 1981 | OT2 | HIS | 253 | 24.275 | 59.480 | 49.790 | 1.00 | 15.87 | DIC |
| ATOM | 1982 | CB | ALA | 258 | 28.094 | 61.904 | 58.825 | 1.00 | 28.44 | DIC |
| ATOM | 1983 | C | ALA | 258 | 29.433 | 64.012 | 58.929 | 1.00 | 27.58 | DIC |
| ATOM | 1984 | O | ALA | 258 | 29.918 | 64.149 | 57.808 | 1.00 | 27.40 | DIC |
| ATOM | 1985 | N | ALA | 258 | 27.094 | 64.069 | 58.174 | 1.00 | 28.06 | DIC |
| ATOM | 1986 | CA | ALA | 258 | 28.048 | 63.405 | 59.105 | 1.00 | 27.73 | DIC |
| ATOM | 1987 | N | TRP | 259 | 30.068 | 64.366 | 60.042 | 1.00 | 27.44 | DIC |
| ATOM | 1988 | CA | TRP | 259 | 31.396 | 64.976 | 60.013 | 1.00 | 26.88 | DIC |
| ATOM | 1989 | CB | TRP | 259 | 31.824 | 65.387 | 61.426 | 1.00 | 27.00 | DIC |
| ATOM | 1990 | CG | TRP | 259 | 33.185 | 66.030 | 61.464 | 1.00 | 26.63 | DIC |
| ATOM | 1991 | CD2 | TRP | 259 | 34.417 | 65.401 | 61.831 | 1.00 | 26.39 | DIC |
| ATOM | 1992 | CE2 | TRP | 259 | 35.440 | 66.363 | 61.676 | 1.00 | 26.47 | DIC |
| ATOM | 1993 | CE3 | TRP | 259 | 34.758 | 64.117 | 62.276 | 1.00 | 26.52 | DIC |
| ATOM | 1994 | CD1 | TRP | 259 | 33.499 | 67.312 | 61.111 | 1.00 | 26.17 | DIC |
| ATOM | 1995 | NE1 | TRP | 259 | 34.853 | 67.520 | 61.236 | 1.00 | 26.25 | DIC |
| ATOM | 1996 | CZ2 | TRP | 259 | 36.779 | 66.080 | 61.948 | 1.00 | 26.25 | DIC |
| ATOM | 1997 | CZ3 | TRP | 259 | 36.090 | 63.837 | 62.546 | 1.00 | 25.96 | DIC |
| ATOM | 1998 | CH2 | TRP | 259 | 37.083 | 64.815 | 62.381 | 1.00 | 26.60 | DIC |
| ATOM | 1999 | C | TRP | 259 | 32.465 | 64.060 | 59.419 | 1.00 | 26.25 | DIC |
| ATOM | 2000 | O | TRP | 259 | 32.511 | 62.869 | 59.723 | 1.00 | 26.62 | DIC |
| ATOM | 2001 | N | GLY | 260 | 33.314 | 64.632 | 58.570 | 1.00 | 25.54 | DIC |
| ATOM | 2002 | CA | GLY | 260 | 34.400 | 63.889 | 57.949 | 1.00 | 24.64 | DIC |
| ATOM | 2003 | C | GLY | 260 | 34.002 | 62.871 | 56.898 | 1.00 | 24.46 | DIC |
| ATOM | 2004 | O | GLY | 260 | 34.833 | 62.072 | 56.462 | 1.00 | 25.49 | DIC |
| ATOM | 2005 | N | ALA | 261 | 32.742 | 62.899 | 56.479 | 1.00 | 24.06 | DIC |
| ATOM | 2006 | CA | ALA | 261 | 32.247 | 61.958 | 55.484 | 1.00 | 23.45 | DIC |
| ATOM | 2007 | CB | ALA | 261 | 30.744 | 62.142 | 55.310 | 1.00 | 23.85 | DIC |
| ATOM | 2008 | C | ALA | 261 | 32.946 | 62.072 | 54.131 | 1.00 | 23.44 | DIC |
| ATOM | 2009 | O | ALA | 261 | 32.868 | 61.161 | 53.315 | 1.00 | 24.03 | DIC |
| ATOM | 2010 | N | ALA | 262 | 33.631 | 63.185 | 53.889 | 1.00 | 23.25 | DIC |
| ATOM | 2011 | CA | ALA | 262 | 34.318 | 63.387 | 52.615 | 1.00 | 23.00 | DIC |
| ATOM | 2012 | CB | ALA | 262 | 34.643 | 64.869 | 52.415 | 1.00 | 23.30 | DIC |
| ATOM | 2013 | C | ALA | 262 | 35.589 | 62.560 | 52.484 | 1.00 | 22.59 | DIC |
| ATOM | 2014 | O | ALA | 262 | 36.061 | 62.312 | 51.374 | 1.00 | 22.28 | DIC |
| ATOM | 2015 | N | ARG | 263 | 36.159 | 62.144 | 53.607 | 1.00 | 21.93 | DIC |
| ATOM | 2016 | CA | ARG | 263 | 37.370 | 61.347 | 53.550 | 1.00 | 21.92 | DIC |
| ATOM | 2017 | CB | ARG | 263 | 37.959 | 61.155 | 54.950 | 1.00 | 24.12 | DIC |
| ATOM | 2018 | CG | ARG | 263 | 39.231 | 60.298 | 54.987 | 1.00 | 27.56 | DIC |
| ATOM | 2019 | CD | ARG | 263 | 39.921 | 60.412 | 56.350 | 1.00 | 30.65 | DIC |
| ATOM | 2020 | NE | ARG | 263 | 40.887 | 59.343 | 56.622 | 1.00 | 33.18 | DIC |
| ATOM | 2021 | CZ | ARG | 263 | 41.943 | 59.055 | 55.862 | 1.00 | 34.55 | DIC |
| ATOM | 2022 | NH1 | ARG | 263 | 42.757 | 58.064 | 56.210 | 1.00 | 35.10 | DIC |
| ATOM | 2023 | NH2 | ARG | 263 | 42.183 | 59.748 | 54.751 | 1.00 | 35.66 | DIC |
| ATOM | 2024 | C | ARG | 263 | 37.062 | 59.998 | 52.906 | 1.00 | 20.84 | DIC |
| ATOM | 2025 | O | ARG | 263 | 37.951 | 59.357 | 52.359 | 1.00 | 19.59 | DIC |
| ATOM | 2026 | N | PHE | 264 | 35.803 | 59.569 | 52.971 | 1.00 | 20.19 | DIC |
| ATOM | 2027 | CA | PHE | 264 | 35.403 | 58.300 | 52.360 | 1.00 | 19.21 | DIC |
| ATOM | 2028 | CB | PHE | 264 | 33.941 | 57.972 | 52.676 | 1.00 | 19.33 | DIC |
| ATOM | 2029 | CG | PHE | 264 | 33.437 | 56.740 | 51.971 | 1.00 | 18.65 | DIC |
| ATOM | 2030 | CD1 | PHE | 264 | 33.600 | 55.480 | 52.536 | 1.00 | 17.83 | DIC |
| ATOM | 2031 | CD2 | PHE | 264 | 32.844 | 56.835 | 50.712 | 1.00 | 17.86 | DIC |
| ATOM | 2032 | CE1 | PHE | 264 | 33.182 | 54.329 | 51.857 | 1.00 | 17.13 | DIC |
| ATOM | 2033 | CE2 | PHE | 264 | 32.427 | 55.694 | 50.031 | 1.00 | 18.25 | DIC |
| ATOM | 2034 | CZ | PHE | 264 | 32.596 | 54.440 | 50.604 | 1.00 | 16.55 | DIC |
| ATOM | 2035 | C | PHE | 264 | 35.560 | 58.371 | 50.844 | 1.00 | 19.58 | DIC |
| ATOM | 2036 | O | PHE | 264 | 36.246 | 57.545 | 50.235 | 1.00 | 18.31 | DIC |
| ATOM | 2037 | N | THR | 265 | 34.909 | 59.357 | 50.233 | 1.00 | 20.22 | DIC |
| ATOM | 2038 | CA | THR | 265 | 34.976 | 59.523 | 48.783 | 1.00 | 21.42 | DIC |
| ATOM | 2039 | CB | THR | 265 | 34.038 | 60.674 | 48.309 | 1.00 | 22.32 | DIC |
| ATOM | 2040 | OG1 | THR | 265 | 34.270 | 60.945 | 46.920 | 1.00 | 23.37 | DIC |
| ATOM | 2041 | CG2 | THR | 265 | 34.268 | 61.927 | 49.111 | 1.00 | 23.03 | DIC |
| ATOM | 2042 | C | THR | 265 | 36.419 | 59.780 | 48.342 | 1.00 | 21.65 | DIC |
| ATOM | 2043 | O | THR | 265 | 36.855 | 59.319 | 47.287 | 1.00 | 20.87 | DIC |
| ATOM | 2044 | N | GLU | 266 | 37.167 | 60.485 | 49.180 | 1.00 | 21.89 | DIC |
| ATOM | 2045 | CA | GLU | 266 | 38.560 | 60.794 | 48.899 | 1.00 | 22.68 | DIC |
| ATOM | 2046 | CB | GLU | 266 | 39.086 | 61.722 | 49.989 | 1.00 | 24.83 | DIC |
| ATOM | 2047 | CG | GLU | 266 | 40.501 | 62.213 | 49.792 | 1.00 | 29.43 | DIC |
| ATOM | 2048 | CD | GLU | 266 | 40.812 | 63.387 | 50.704 | 1.00 | 31.88 | DIC |
| ATOM | 2049 | OE1 | GLU | 266 | 40.454 | 63.314 | 51.903 | 1.00 | 32.56 | DIC |
| ATOM | 2050 | OE2 | GLU | 266 | 41.410 | 64.380 | 50.223 | 1.00 | 33.53 | DIC |
| ATOM | 2051 | C | GLU | 266 | 39.394 | 59.504 | 48.824 | 1.00 | 21.84 | DIC |
| ATOM | 2052 | O | GLU | 266 | 40.229 | 59.348 | 47.935 | 1.00 | 20.75 | DIC |
| ATOM | 2053 | N | LEU | 267 | 39.162 | 58.584 | 49.760 | 1.00 | 19.85 | DIC |
| ATOM | 2054 | CA | LEU | 267 | 39.874 | 57.308 | 49.780 | 1.00 | 19.01 | DIC |
| ATOM | 2055 | CB | LEU | 267 | 39.643 | 56.581 | 51.105 | 1.00 | 18.80 | DIC |
| ATOM | 2056 | CG | LEU | 267 | 40.471 | 57.095 | 52.276 | 1.00 | 18.91 | DIC |
| ATOM | 2057 | CD1 | LEU | 267 | 39.894 | 56.591 | 53.588 | 1.00 | 18.99 | DIC |
| ATOM | 2058 | CD2 | LEU | 267 | 41.914 | 56.654 | 52.082 | 1.00 | 19.67 | DIC |
| ATOM | 2059 | C | LEU | 267 | 39.406 | 56.425 | 48.639 | 1.00 | 18.45 | DIC |
| ATOM | 2060 | O | LEU | 267 | 40.215 | 55.764 | 47.992 | 1.00 | 16.88 | DIC |
| ATOM | 2061 | N | ALA | 268 | 38.095 | 56.408 | 48.398 | 1.00 | 18.86 | DIC |
| ATOM | 2062 | CA | ALA | 268 | 37.532 | 55.607 | 47.318 | 1.00 | 20.48 | DIC |
| ATOM | 2063 | CB | ALA | 268 | 36.018 | 55.792 | 47.254 | 1.00 | 21.06 | DIC |
| ATOM | 2064 | C | ALA | 268 | 38.169 | 56.024 | 45.996 | 1.00 | 21.58 | DIC |
| ATOM | 2065 | O | ALA | 268 | 38.481 | 55.185 | 45.157 | 1.00 | 20.74 | DIC |
| ATOM | 2066 | N | GLY | 269 | 38.376 | 57.324 | 45.820 | 1.00 | 22.51 | DIC |
| ATOM | 2067 | CA | GLY | 269 | 38.979 | 57.805 | 44.590 | 1.00 | 24.41 | DIC |
| ATOM | 2068 | C | GLY | 269 | 40.444 | 57.429 | 44.473 | 1.00 | 25.41 | DIC |
| ATOM | 2069 | O | GLY | 269 | 41.007 | 57.421 | 43.379 | 1.00 | 25.74 | DIC |
| ATOM | 2070 | N | SER | 270 | 41.069 | 57.114 | 45.600 | 1.00 | 25.49 | DIC |
| ATOM | 2071 | CA | SER | 270 | 42.479 | 56.748 | 45.598 | 1.00 | 26.14 | DIC |
| ATOM | 2072 | CB | SER | 270 | 43.117 | 57.107 | 46.939 | 1.00 | 25.67 | DIC |
| ATOM | 2073 | OG | SER | 270 | 42.648 | 56.249 | 47.965 | 1.00 | 25.26 | DIC |
| ATOM | 2074 | C | SER | 270 | 42.689 | 55.263 | 45.329 | 1.00 | 26.42 | DIC |
| ATOM | 2075 | O | SER | 270 | 43.821 | 54.815 | 45.155 | 1.00 | 26.83 | DIC |
| ATOM | 2076 | N | LEU | 271 | 41.603 | 54.498 | 45.298 | 1.00 | 26.90 | DIC |
| ATOM | 2077 | CA | LEU | 271 | 41.708 | 53.063 | 45.065 | 1.00 | 27.83 | DIC |
| ATOM | 2078 | CB | LEU | 271 | 40.319 | 52.417 | 45.034 | 1.00 | 27.48 | DIC |
| ATOM | 2079 | CG | LEU | 271 | 39.575 | 52.392 | 46.367 | 1.00 | 27.23 | DIC |
| ATOM | 2080 | CD1 | LEU | 271 | 38.288 | 51.595 | 46.216 | 1.00 | 26.49 | DIC |
| ATOM | 2081 | CD2 | LEU | 271 | 40.470 | 51.778 | 47.435 | 1.00 | 26.56 | DIC |
| ATOM | 2082 | C | LEU | 271 | 42.460 | 52.696 | 43.793 | 1.00 | 28.34 | DIC |
| ATOM | 2083 | O | LEU | 271 | 42.286 | 53.314 | 42.744 | 1.00 | 28.17 | DIC |
| ATOM | 2084 | N | THR | 272 | 43.289 | 51.665 | 43.907 | 1.00 | 29.25 | DIC |
| ATOM | 2085 | CA | THR | 272 | 44.088 | 51.166 | 42.799 | 1.00 | 29.35 | DIC |
| ATOM | 2086 | CB | THR | 272 | 45.106 | 50.129 | 43.293 | 1.00 | 29.61 | DIC |
| ATOM | 2087 | OG1 | THR | 272 | 45.930 | 50.712 | 44.312 | 1.00 | 30.29 | DIC |
| ATOM | 2088 | CG2 | THR | 272 | 45.974 | 49.654 | 42.142 | 1.00 | 29.87 | DIC |
| ATOM | 2089 | C | THR | 272 | 43.229 | 50.500 | 41.728 | 1.00 | 29.37 | DIC |
| ATOM | 2090 | O | THR | 272 | 43.324 | 50.838 | 40.548 | 1.00 | 29.51 | DIC |
| ATOM | 2091 | N | THR | 273 | 42.409 | 49.541 | 42.151 | 1.00 | 28.69 | DIC |
| ATOM | 2092 | CA | THR | 273 | 41.533 | 48.798 | 41.248 | 1.00 | 28.59 | DIC |
| ATOM | 2093 | CB | THR | 273 | 41.892 | 47.297 | 41.218 | 1.00 | 30.32 | DIC |
| ATOM | 2094 | OG1 | THR | 273 | 43.302 | 47.137 | 41.014 | 1.00 | 32.39 | DIC |
| ATOM | 2095 | CG2 | THR | 273 | 41.140 | 46.602 | 40.096 | 1.00 | 31.41 | DIC |
| ATOM | 2096 | C | THR | 273 | 40.077 | 48.896 | 41.690 | 1.00 | 27.15 | DIC |
| ATOM | 2097 | O | THR | 273 | 39.746 | 48.572 | 42.831 | 1.00 | 26.61 | DIC |
| ATOM | 2098 | N | VAL | 274 | 39.204 | 49.334 | 40.789 | 1.00 | 24.82 | DIC |
| ATOM | 2099 | CA | VAL | 274 | 37.792 | 49.445 | 41.128 | 1.00 | 23.71 | DIC |
| ATOM | 2100 | CB | VAL | 274 | 37.361 | 50.924 | 41.261 | 1.00 | 24.47 | DIC |
| ATOM | 2101 | CG1 | VAL | 274 | 37.742 | 51.692 | 40.014 | 1.00 | 24.70 | DIC |
| ATOM | 2102 | CG2 | VAL | 274 | 35.862 | 51.005 | 41.509 | 1.00 | 24.49 | DIC |
| ATOM | 2103 | C | VAL | 274 | 36.917 | 48.739 | 40.095 | 1.00 | 22.44 | DIC |
| ATOM | 2104 | O | VAL | 274 | 36.769 | 49.202 | 38.964 | 1.00 | 21.71 | DIC |
| ATOM | 2105 | N | PRO | 275 | 36.341 | 47.587 | 40.477 | 1.00 | 21.18 | DIC |
| ATOM | 2106 | CD | PRO | 275 | 36.537 | 46.929 | 41.779 | 1.00 | 20.08 | DIC |
| ATOM | 2107 | CA | PRO | 275 | 35.472 | 46.778 | 39.619 | 1.00 | 20.90 | DIC |
| ATOM | 2108 | CB | PRO | 275 | 35.053 | 45.630 | 40.534 | 1.00 | 20.65 | DIC |
| ATOM | 2109 | CG | PRO | 275 | 36.203 | 45.498 | 41.464 | 1.00 | 20.01 | DIC |
| ATOM | 2110 | C | PRO | 275 | 34.267 | 47.558 | 39.120 | 1.00 | 20.92 | DIC |
| ATOM | 2111 | O | PRO | 275 | 33.714 | 48.397 | 39.835 | 1.00 | 20.31 | DIC |
| ATOM | 2112 | N | GLU | 276 | 33.859 | 47.273 | 37.890 | 1.00 | 22.03 | DIC |
| ATOM | 2113 | CA | GLU | 276 | 32.701 | 47.938 | 37.312 | 1.00 | 22.81 | DIC |
| ATOM | 2114 | CB | GLU | 276 | 32.317 | 47.269 | 35.987 | 1.00 | 23.16 | DIC |
| ATOM | 2115 | CG | GLU | 276 | 30.944 | 47.665 | 35.449 | 1.00 | 23.75 | DIC |
| ATOM | 2116 | CD | GLU | 276 | 30.859 | 49.123 | 35.047 | 1.00 | 23.43 | DIC |
| ATOM | 2117 | OE1 | GLU | 276 | 31.868 | 49.846 | 35.176 | 1.00 | 24.18 | DIC |
| ATOM | 2118 | OE2 | GLU | 276 | 29.774 | 49.547 | 34.598 | 1.00 | 24.32 | DIC |
| ATOM | 2119 | C | GLU | 276 | 31.540 | 47.843 | 38.293 | 1.00 | 23.40 | DIC |
| ATOM | 2120 | O | GLU | 276 | 30.810 | 48.811 | 38.496 | 1.00 | 24.06 | DIC |
| ATOM | 2121 | N | GLU | 277 | 31.395 | 46.683 | 38.928 | 1.00 | 23.55 | DIC |
| ATOM | 2122 | CA | GLU | 277 | 30.303 | 46.472 | 39.865 | 1.00 | 24.83 | DIC |
| ATOM | 2123 | CB | GLU | 277 | 30.148 | 44.976 | 40.157 | 1.00 | 27.38 | DIC |
| ATOM | 2124 | CG | GLU | 277 | 28.685 | 44.509 | 40.248 | 1.00 | 31.14 | DIC |
| ATOM | 2125 | CD | GLU | 277 | 28.029 | 44.257 | 38.882 | 1.00 | 33.24 | DIC |
| ATOM | 2126 | OE1 | GLU | 277 | 28.023 | 45.164 | 38.017 | 1.00 | 34.02 | DIC |
| ATOM | 2127 | OE2 | GLU | 277 | 27.508 | 43.136 | 38.679 | 1.00 | 34.95 | DIC |
| ATOM | 2128 | C | GLU | 277 | 30.419 | 47.255 | 41.180 | 1.00 | 23.83 | DIC |
| ATOM | 2129 | O | GLU | 277 | 29.507 | 47.209 | 42.009 | 1.00 | 24.63 | DIC |
| ATOM | 2130 | N | TRP | 278 | 31.532 | 47.964 | 41.373 | 1.00 | 22.09 | DIC |
| ATOM | 2131 | CA | TRP | 278 | 31.735 | 48.775 | 42.580 | 1.00 | 20.45 | DIC |
| ATOM | 2132 | CB | TRP | 278 | 33.183 | 48.680 | 43.078 | 1.00 | 17.96 | DIC |
| ATOM | 2133 | CG | TRP | 278 | 33.544 | 47.431 | 43.835 | 1.00 | 16.02 | DIC |
| ATOM | 2134 | CD2 | TRP | 278 | 34.693 | 47.245 | 44.677 | 1.00 | 16.17 | DIC |
| ATOM | 2135 | CE2 | TRP | 278 | 34.676 | 45.906 | 45.117 | 1.00 | 16.04 | DIC |
| ATOM | 2136 | CE3 | TRP | 278 | 35.738 | 48.084 | 45.101 | 1.00 | 16.30 | DIC |
| ATOM | 2137 | CD1 | TRP | 278 | 32.897 | 46.235 | 43.804 | 1.00 | 15.72 | DIC |
| ATOM | 2138 | NE1 | TRP | 278 | 33.569 | 45.312 | 44.568 | 1.00 | 15.84 | DIC |
| ATOM | 2139 | CZ2 | TRP | 278 | 35.665 | 45.378 | 45.959 | 1.00 | 15.03 | DIC |
| ATOM | 2140 | CZ3 | TRP | 278 | 36.728 | 47.557 | 45.941 | 1.00 | 15.75 | DIC |
| ATOM | 2141 | CH2 | TRP | 278 | 36.679 | 46.216 | 46.360 | 1.00 | 16.12 | DIC |
| ATOM | 2142 | C | TRP | 278 | 31.453 | 50.236 | 42.248 | 1.00 | 20.70 | DIC |
| ATOM | 2143 | O | TRP | 278 | 31.281 | 51.066 | 43.141 | 1.00 | 20.71 | DIC |
| ATOM | 2144 | N | ALA | 279 | 31.416 | 50.542 | 40.955 | 1.00 | 21.39 | DIC |
| ATOM | 2145 | CA | ALA | 279 | 31.190 | 51.904 | 40.479 | 1.00 | 22.71 | DIC |
| ATOM | 2146 | CB | ALA | 279 | 31.084 | 51.910 | 38.954 | 1.00 | 23.51 | DIC |
| ATOM | 2147 | C | ALA | 279 | 29.965 | 52.563 | 41.087 | 1.00 | 23.55 | DIC |
| ATOM | 2148 | O | ALA | 279 | 29.959 | 53.771 | 41.323 | 1.00 | 24.28 | DIC |
| ATOM | 2149 | N | GLY | 280 | 28.929 | 51.768 | 41.333 | 1.00 | 24.37 | DIC |
| ATOM | 2150 | CA | GLY | 280 | 27.705 | 52.294 | 41.913 | 1.00 | 25.98 | DIC |
| ATOM | 2151 | C | GLY | 280 | 27.849 | 52.651 | 43.383 | 1.00 | 26.65 | DIC |
| ATOM | 2152 | O | GLY | 280 | 27.674 | 53.808 | 43.770 | 1.00 | 27.02 | DIC |
| ATOM | 2153 | N | ALA | 281 | 28.165 | 51.657 | 44.208 | 1.00 | 27.42 | DIC |
| ATOM | 2154 | CA | ALA | 281 | 28.337 | 51.874 | 45.639 | 1.00 | 28.02 | DIC |
| ATOM | 2155 | CB | ALA | 281 | 28.645 | 50.550 | 46.334 | 1.00 | 27.29 | DIC |
| ATOM | 2156 | C | ALA | 281 | 29.477 | 52.860 | 45.857 | 1.00 | 28.66 | DIC |
| ATOM | 2157 | O | ALA | 281 | 29.529 | 53.550 | 46.874 | 1.00 | 29.25 | DIC |
| ATOM | 2158 | N | ALA | 282 | 30.377 | 52.912 | 44.879 | 1.00 | 29.42 | DIC |
| ATOM | 2159 | CA | ALA | 282 | 31.552 | 53.784 | 44.887 | 1.00 | 30.55 | DIC |
| ATOM | 2160 | CB | ALA | 282 | 31.121 | 55.261 | 44.884 | 1.00 | 30.21 | DIC |
| ATOM | 2161 | C | ALA | 282 | 32.458 | 53.501 | 46.078 | 1.00 | 31.20 | DIC |
| ATOM | 2162 | OT1 | ALA | 282 | 33.390 | 52.669 | 45.938 | 1.00 | 32.04 | DIC |
| ATOM | 2163 | OT2 | ALA | 282 | 32.207 | 54.100 | 47.145 | 1.00 | 31.25 | DIC |
| ATOM | 2164 | N1 | UPG | 341 | 27.240 | 44.499 | 57.825 | 1.00 | 10.18 | |
| ATOM | 2165 | C2 | UPG | 341 | 27.623 | 43.818 | 56.650 | 1.00 | 9.69 | |
| ATOM | 2166 | N3 | UPG | 341 | 26.746 | 44.059 | 55.575 | 1.00 | 8.84 | |
| ATOM | 2167 | C4 | UPG | 341 | 25.566 | 44.894 | 55.582 | 1.00 | 8.96 | |
| ATOM | 2168 | C5 | UPG | 341 | 25.252 | 45.558 | 56.815 | 1.00 | 8.24 | |
| ATOM | 2169 | C6 | UPG | 341 | 26.056 | 45.357 | 57.869 | 1.00 | 8.97 | |
| ATOM | 2170 | O2 | UPG | 341 | 28.602 | 43.097 | 56.579 | 1.00 | 9.27 | |
| ATOM | 2171 | O4 | UPG | 341 | 24.905 | 44.993 | 54.563 | 1.00 | 11.09 | |
| ATOM | 2172 | C4* | UPG | 341 | 28.140 | 45.874 | 61.110 | 1.00 | 9.66 | |
| ATOM | 2173 | O4* | UPG | 341 | 27.321 | 44.837 | 60.426 | 1.00 | 9.94 | |
| ATOM | 2174 | C3* | UPG | 341 | 28.902 | 46.508 | 59.951 | 1.00 | 8.28 | |
| ATOM | 2175 | O3* | UPG | 341 | 29.996 | 47.267 | 60.429 | 1.00 | 10.54 | |
| ATOM | 2176 | C2* | UPG | 341 | 29.173 | 45.283 | 59.098 | 1.00 | 9.24 | |
| ATOM | 2177 | O2* | UPG | 341 | 30.290 | 44.505 | 59.538 | 1.00 | 9.22 | |
| ATOM | 2178 | C1* | UPG | 341 | 27.913 | 44.478 | 59.203 | 1.00 | 10.37 | |
| ATOM | 2179 | C5* | UPG | 341 | 26.834 | 46.274 | 61.715 | 1.00 | 8.33 | |
| ATOM | 2180 | O5* | UPG | 341 | 26.278 | 47.636 | 61.455 | 1.00 | 14.17 | |
| ATOM | 2181 | PA | UPG | 341 | 26.757 | 49.151 | 61.231 | 1.00 | 11.35 | |
| ATOM | 2182 | O1A | UPG | 341 | 28.224 | 49.345 | 61.258 | 1.00 | 12.96 | |
| ATOM | 2183 | O2A | UPG | 341 | 26.106 | 49.722 | 60.020 | 1.00 | 14.14 | |
| ATOM | 2184 | O3A | UPG | 341 | 25.990 | 49.305 | 62.626 | 1.00 | 12.31 | |
| ATOM | 2185 | PB | UPG | 341 | 25.718 | 50.499 | 63.635 | 1.00 | 14.51 | |
| ATOM | 2186 | O1B | UPG | 341 | 27.055 | 51.134 | 63.729 | 1.00 | 11.54 | |
| ATOM | 2187 | O2B | UPG | 341 | 24.736 | 51.411 | 63.036 | 1.00 | 15.63 | |
| ATOM | 2188 | O3B | UPG | 341 | 25.289 | 49.955 | 65.021 | 1.00 | 15.47 | |
| ATOM | 2189 | C1′ | UPG | 341 | 25.701 | 49.765 | 66.543 | 1.00 | 20.67 | |
| ATOM | 2190 | C2′ | UPG | 341 | 27.182 | 49.323 | 66.490 | 1.00 | 19.41 | |
| ATOM | 2191 | C3′ | UPG | 341 | 27.600 | 48.129 | 65.515 | 1.00 | 20.26 | |
| ATOM | 2192 | C4′ | UPG | 341 | 26.609 | 46.923 | 65.726 | 1.00 | 19.77 | |
| ATOM | 2193 | C5′ | UPG | 341 | 25.121 | 47.545 | 65.719 | 1.00 | 20.08 | |
| ATOM | 2194 | C6′ | UPG | 341 | 24.111 | 46.486 | 66.179 | 1.00 | 21.34 | |
| ATOM | 2195 | F2′ | UPG | 341 | 28.054 | 50.401 | 66.263 | 1.00 | 22.29 | |
| ATOM | 2196 | O3′ | UPG | 341 | 28.884 | 47.722 | 65.901 | 1.00 | 19.02 | |
| ATOM | 2197 | O4′ | UPG | 341 | 26.927 | 46.281 | 66.991 | 1.00 | 18.02 | |
| ATOM | 2198 | O5′ | UPG | 341 | 24.861 | 48.621 | 66.750 | 1.00 | 20.07 | |
| ATOM | 2199 | O6′ | UPG | 341 | 23.853 | 45.237 | 65.594 | 1.00 | 20.34 | |
| ATOM | 2200 | MN | MN | 400 | 29.038 | 50.750 | 62.751 | 1.00 | 6.12 | MN |
| ATOM | 2201 | C1 | LAT | 1347 | 21.862 | 53.689 | 65.707 | 1.00 | 20.18 | |
| ATOM | 2202 | C2 | LAT | 1347 | 22.118 | 52.340 | 65.066 | 1.00 | 20.18 | |
| ATOM | 2203 | C3 | LAT | 1347 | 22.177 | 51.325 | 66.179 | 1.00 | 20.18 | |
| ATOM | 2204 | C4 | LAT | 1347 | 23.332 | 51.731 | 67.165 | 1.00 | 20.18 | |
| ATOM | 2205 | C5 | LAT | 1347 | 23.165 | 53.016 | 67.800 | 1.00 | 20.18 | |
| ATOM | 2206 | C6 | LAT | 1347 | 24.129 | 53.435 | 68.634 | 1.00 | 20.18 | |
| ATOM | 2207 | O1 | LAT | 1347 | 21.580 | 54.987 | 65.275 | 1.00 | 19.89 | |
| ATOM | 2208 | O2 | LAT | 1347 | 21.058 | 51.980 | 64.134 | 1.00 | 20.18 | |
| ATOM | 2209 | O3 | LAT | 1347 | 22.433 | 50.083 | 65.582 | 1.00 | 20.18 | |
| ATOM | 2210 | O5 | LAT | 1347 | 22.941 | 54.020 | 66.702 | 1.00 | 20.18 | |
| ATOM | 2211 | O6 | LAT | 1347 | 24.638 | 52.525 | 69.648 | 1.00 | 20.18 | |
| ATOM | 2212 | C1′ | LAT | 1347 | 20.888 | 59.061 | 65.028 | 1.00 | 19.89 | |
| ATOM | 2213 | C2′ | LAT | 1347 | 22.121 | 58.647 | 65.845 | 1.00 | 19.89 | |
| ATOM | 2214 | C3′ | LAT | 1347 | 22.505 | 57.214 | 65.448 | 1.00 | 19.89 | |
| ATOM | 2215 | C4′ | LAT | 1347 | 21.315 | 56.296 | 65.729 | 1.00 | 19.89 | |
| ATOM | 2216 | C5′ | LAT | 1347 | 20.008 | 56.750 | 65.020 | 1.00 | 19.89 | |
| ATOM | 2217 | C6′ | LAT | 1347 | 18.753 | 55.952 | 65.464 | 1.00 | 19.89 | |
| ATOM | 2218 | O1′ | LAT | 1347 | 20.572 | 60.386 | 65.395 | 1.00 | 19.89 | |
| ATOM | 2219 | O2′ | LAT | 1347 | 23.194 | 59.542 | 65.568 | 1.00 | 19.89 | |
| ATOM | 2220 | O3′ | LAT | 1347 | 23.637 | 56.854 | 66.255 | 1.00 | 19.89 | |
| ATOM | 2221 | O5′ | LAT | 1347 | 19.798 | 58.147 | 65.342 | 1.00 | 19.89 | |
| ATOM | 2222 | O6′ | LAT | 1347 | 18.292 | 55.063 | 64.439 | 1.00 | 19.89 | |

TABLE 6
REMARK coordinates from minimization refinement
REMARK refinement resolution: 20.0-2.0 A
REMARK starting r = .1986 free_r = .2234
REMARK final r = .1949 free_r = .2236
REMARK rmsd bonds = .006530 rmsd angles = 1.33814
REMARK wa = 1.2
REMARK target = mlf cycles = 1 steps = 20
REMARK sg = P2(1)2(1)2(1) a = 39.953 b = 76.126 c = 87.163
alpha = 90 beta = 90 gamma = 90
REMARK parameter file 1: CNS_TOPPAR:protein_rep.param
REMARK parameter file 2: ../rnd5/upg.par
REMARK parameter file 3: CNS_TOPPAR:ion.param
REMARK parameter file 4: CNS_TOPPAR:water_rep.param
REMARK parameter file 5: ../lat.par
REMARK molecular structure file: generate8_2.mtf
REMARK input coordinates: generate8_2.pdb
REMARK reflection file = ../lac1.cv
REMARK ncs = none
REMARK B-correction resolution: 6.0-2.0
REMARK initial B-factor correction applied to fobs:
REMARK B11 = 2.769 B22 = 1.768 B33 = −4.536
REMARK B12 = .000 B13 = .000 B23 = .000
REMARK B-factor correction applied to coordinate array B: .038
REMARK bulk solvent: density level = .333397 e/A 3, B-factor = 34.3204 A 2
REMARK reflections with |Fobs|/sigma_F <0.0 rejected
REMARK reflections with |Fobs| > 10000 * rms (Fobs) rejected

| | |
|---|---|
| REMARK theoretical total number of refl. in resol. range: | 18567 ( 100.0% ) |
| REMARK number of unobserved reflections (no entry or \|F\| = 0): | 337 ( 1.8% ) |
| REMARK number of reflections rejected: | 0 ( .0% ) |
| REMARK total number of reflections used: | 18230 ( 98.2% ) |
| REMARK number of reflections in working set: | 17343 ( 93.4% ) |
| REMARK number of reflections in test set: | 887 ( 4.8% ) |

CRYST1 39.953 76.126 87.163 90.00 90.00 90.00 P 21 21 21
REMARK FILENAME = “minimize8_2.pdb”
REMARK DATE: 8-Nov-00 23:09:42 created by user: karina
REMARK VERSION: 1.0

| | | | | | | | | | | |
|---|---|---|---|---|---|---|---|---|---|---|
| ATOM | 1 | CB | MSE | 1 | 48.127 | 46.242 | 65.128 | 1.00 | 17.58 | DIC |
| ATOM | 2 | CG | MSE | 1 | 47.640 | 47.179 | 64.037 | 1.00 | 20.74 | DIC |
| ATOM | 3 | SE | MSE | 1 | 46.763 | 48.808 | 64.758 | 1.00 | 18.31 | DIC |
| ATOM | 4 | CE | MSE | 1 | 48.354 | 49.664 | 65.452 | 1.00 | 19.80 | DIC |
| ATOM | 5 | C | MSE | 1 | 47.777 | 44.093 | 63.871 | 1.00 | 13.39 | DIC |
| ATOM | 6 | O | MSE | 1 | 47.840 | 44.063 | 62.643 | 1.00 | 13.12 | DIC |
| ATOM | 7 | N | MSE | 1 | 49.999 | 45.167 | 63.882 | 1.00 | 14.29 | DIC |
| ATOM | 8 | CA | MSE | 1 | 48.769 | 44.914 | 64.676 | 1.00 | 14.05 | DIC |
| ATOM | 9 | N | ASP | 2 | 46.876 | 43.408 | 64.566 | 1.00 | 13.55 | DIC |
| ATOM | 10 | CA | ASP | 2 | 45.870 | 42.590 | 63.901 | 1.00 | 13.67 | DIC |
| ATOM | 11 | CB | ASP | 2 | 45.770 | 41.211 | 64.547 | 1.00 | 14.41 | DIC |
| ATOM | 12 | CG | ASP | 2 | 47.030 | 40.392 | 64.352 | 1.00 | 15.71 | DIC |
| ATOM | 13 | OD1 | ASP | 2 | 47.932 | 40.477 | 65.207 | 1.00 | 14.07 | DIC |
| ATOM | 14 | OD2 | ASP | 2 | 47.121 | 39.682 | 63.329 | 1.00 | 16.48 | DIC |
| ATOM | 15 | C | ASP | 2 | 44.519 | 43.273 | 63.942 | 1.00 | 14.09 | DIC |
| ATOM | 16 | O | ASP | 2 | 43.974 | 43.544 | 65.012 | 1.00 | 13.05 | DIC |
| ATOM | 17 | N | ILE | 3 | 43.994 | 43.562 | 62.758 | 1.00 | 12.65 | DIC |
| ATOM | 18 | CA | ILE | 3 | 42.707 | 44.217 | 62.624 | 1.00 | 11.17 | DIC |
| ATOM | 19 | CB | ILE | 3 | 42.748 | 45.331 | 61.560 | 1.00 | 10.15 | DIC |
| ATOM | 20 | CG2 | ILE | 3 | 41.355 | 45.938 | 61.402 | 1.00 | 8.26 | DIC |
| ATOM | 21 | CG1 | ILE | 3 | 43.816 | 46.371 | 61.921 | 1.00 | 9.18 | DIC |
| ATOM | 22 | CD | ILE | 3 | 43.553 | 47.140 | 63.221 | 1.00 | 10.15 | DIC |
| ATOM | 23 | C | ILE | 3 | 41.699 | 43.186 | 62.169 | 1.00 | 11.33 | DIC |
| ATOM | 24 | O | ILE | 3 | 42.021 | 42.313 | 61.364 | 1.00 | 12.76 | DIC |
| ATOM | 25 | N | VAL | 4 | 40.481 | 43.293 | 62.682 | 1.00 | 11.00 | DIC |
| ATOM | 26 | CA | VAL | 4 | 39.420 | 42.375 | 62.305 | 1.00 | 11.29 | DIC |
| ATOM | 27 | CB | VAL | 4 | 38.923 | 41.545 | 63.501 | 1.00 | 12.03 | DIC |
| ATOM | 28 | CG1 | VAL | 4 | 37.776 | 40.629 | 63.046 | 1.00 | 13.87 | DIC |
| ATOM | 29 | CG2 | VAL | 4 | 40.068 | 40.719 | 64.083 | 1.00 | 12.49 | DIC |
| ATOM | 30 | C | VAL | 4 | 38.226 | 43.144 | 61.758 | 1.00 | 10.90 | DIC |
| ATOM | 31 | O | VAL | 4 | 37.808 | 44.146 | 62.332 | 1.00 | 12.17 | DIC |
| ATOM | 32 | N | PHE | 5 | 37.704 | 42.676 | 60.630 | 1.00 | 11.30 | DIC |
| ATOM | 33 | CA | PHE | 5 | 36.524 | 43.259 | 59.998 | 1.00 | 9.47 | DIC |
| ATOM | 34 | CB | PHE | 5 | 36.862 | 43.876 | 58.635 | 1.00 | 11.11 | DIC |
| ATOM | 35 | CG | PHE | 5 | 37.513 | 45.229 | 58.702 | 1.00 | 9.37 | DIC |
| ATOM | 36 | CD1 | PHE | 5 | 36.818 | 46.332 | 59.186 | 1.00 | 10.57 | DIC |
| ATOM | 37 | CD2 | PHE | 5 | 38.799 | 45.412 | 58.204 | 1.00 | 10.04 | DIC |
| ATOM | 38 | CE1 | PHE | 5 | 37.398 | 47.610 | 59.164 | 1.00 | 11.62 | DIC |
| ATOM | 39 | CE2 | PHE | 5 | 39.386 | 46.674 | 58.178 | 1.00 | 11.49 | DIC |
| ATOM | 40 | CZ | PHE | 5 | 38.683 | 47.776 | 58.657 | 1.00 | 12.84 | DIC |
| ATOM | 41 | C | PHE | 5 | 35.589 | 42.074 | 59.748 | 1.00 | 10.75 | DIC |
| ATOM | 42 | O | PHE | 5 | 36.034 | 40.922 | 59.713 | 1.00 | 9.51 | DIC |
| ATOM | 43 | N | ALA | 6 | 34.305 | 42.364 | 59.581 | 1.00 | 8.88 | DIC |
| ATOM | 44 | CA | ALA | 6 | 33.296 | 41.353 | 59.281 | 1.00 | 9.05 | DIC |
| ATOM | 45 | CB | ALA | 6 | 32.408 | 41.090 | 60.493 | 1.00 | 7.20 | DIC |
| ATOM | 46 | C | ALA | 6 | 32.461 | 41.947 | 58.154 | 1.00 | 10.16 | DIC |
| ATOM | 47 | O | ALA | 6 | 32.012 | 43.093 | 58.247 | 1.00 | 9.64 | DIC |
| ATOM | 48 | N | ALA | 7 | 32.244 | 41.184 | 57.093 | 1.00 | 9.74 | DIC |
| ATOM | 49 | CA | ALA | 7 | 31.451 | 41.699 | 55.987 | 1.00 | 11.40 | DIC |
| ATOM | 50 | CB | ALA | 7 | 32.306 | 42.623 | 55.126 | 1.00 | 10.99 | DIC |
| ATOM | 51 | C | ALA | 7 | 30.852 | 40.616 | 55.113 | 1.00 | 11.73 | DIC |
| ATOM | 52 | O | ALA | 7 | 31.415 | 39.528 | 54.987 | 1.00 | 11.58 | DIC |
| ATOM | 53 | N | ASP | 8 | 29.686 | 40.914 | 54.542 | 1.00 | 11.95 | DIC |
| ATOM | 54 | CA | ASP | 8 | 29.040 | 40.005 | 53.609 | 1.00 | 10.87 | DIC |
| ATOM | 55 | CB | ASP | 8 | 27.516 | 39.942 | 53.812 | 1.00 | 11.16 | DIC |
| ATOM | 56 | CG | ASP | 8 | 26.892 | 41.297 | 54.079 | 1.00 | 11.92 | DIC |
| ATOM | 57 | OD1 | ASP | 8 | 27.415 | 42.320 | 53.585 | 1.00 | 9.02 | DIC |
| ATOM | 58 | OD2 | ASP | 8 | 25.855 | 41.326 | 54.779 | 1.00 | 12.31 | DIC |
| ATOM | 59 | C | ASP | 8 | 29.377 | 40.605 | 52.249 | 1.00 | 12.11 | DIC |
| ATOM | 60 | O | ASP | 8 | 30.072 | 41.621 | 52.176 | 1.00 | 12.03 | DIC |
| ATOM | 61 | N | ASP | 9 | 28.895 | 40.006 | 51.170 | 1.00 | 12.52 | DIC |
| ATOM | 62 | CA | ASP | 9 | 29.232 | 40.523 | 49.849 | 1.00 | 13.97 | DIC |
| ATOM | 63 | CB | ASP | 9 | 28.640 | 39.630 | 48.759 | 1.00 | 15.00 | DIC |
| ATOM | 64 | CG | ASP | 9 | 29.274 | 39.883 | 47.412 | 1.00 | 15.24 | DIC |
| ATOM | 65 | OD1 | ASP | 9 | 30.490 | 39.628 | 47.269 | 1.00 | 16.40 | DIC |
| ATOM | 66 | OD2 | ASP | 9 | 28.567 | 40.349 | 46.502 | 1.00 | 19.30 | DIC |
| ATOM | 67 | C | ASP | 9 | 28.808 | 41.970 | 49.613 | 1.00 | 14.35 | DIC |
| ATOM | 68 | O | ASP | 9 | 29.514 | 42.727 | 48.948 | 1.00 | 14.40 | DIC |
| ATOM | 69 | N | ASN | 10 | 27.660 | 42.352 | 50.164 | 1.00 | 14.55 | DIC |
| ATOM | 70 | CA | ASN | 10 | 27.135 | 43.704 | 50.010 | 1.00 | 15.08 | DIC |
| ATOM | 71 | CB | ASN | 10 | 25.792 | 43.817 | 50.738 | 1.00 | 15.92 | DIC |
| ATOM | 72 | CG | ASN | 10 | 25.151 | 45.191 | 50.593 | 1.00 | 16.37 | DIC |
| ATOM | 73 | OD1 | ASN | 10 | 24.987 | 45.697 | 49.486 | 1.00 | 15.05 | DIC |
| ATOM | 74 | ND2 | ASN | 10 | 24.772 | 45.791 | 51.719 | 1.00 | 15.34 | DIC |
| ATOM | 75 | C | ASN | 10 | 28.084 | 44.796 | 50.513 | 1.00 | 15.42 | DIC |
| ATOM | 76 | O | ASN | 10 | 28.065 | 45.922 | 50.007 | 1.00 | 14.99 | DIC |
| ATOM | 77 | N | TYR | 11 | 28.918 | 44.468 | 51.496 | 1.00 | 14.68 | DIC, |
| ATOM | 78 | CA | TYR | 11 | 29.846 | 45.453 | 52.063 | 1.00 | 13.78 | DIC |
| ATOM | 79 | CB | TYR | 11 | 29.746 | 45.436 | 53.592 | 1.00 | 13.14 | DIC |
| ATOM | 80 | CG | TYR | 11 | 28.744 | 46.423 | 54.138 | 1.00 | 15.36 | DIC |
| ATOM | 81 | CD1 | TYR | 11 | 27.647 | 46.820 | 53.376 | 1.00 | 15.46 | DIC |
| ATOM | 82 | CE1 | TYR | 11 | 26.735 | 47.745 | 53.859 | 1.00 | 16.70 | DIC |
| ATOM | 83 | CD2 | TYR | 11 | 28.900 | 46.976 | 55.409 | 1.00 | 15.54 | DIC |
| ATOM | 84 | CE2 | TYR | 11 | 27.988 | 47.902 | 55.904 | 1.00 | 18.09 | DIC |
| ATOM | 85 | CZ | TYR | 11 | 26.909 | 48.281 | 55.120 | 1.00 | 17.62 | DIC |
| ATOM | 86 | OH | TYR | 11 | 26.005 | 49.200 | 55.585 | 1.00 | 19.94 | DIC |
| ATOM | 87 | C | TYR | 11 | 31.300 | 45.295 | 51.647 | 1.00 | 13.79 | DIC |
| ATOM | 88 | O | TYR | 11 | 32.183 | 45.965 | 52.193 | 1.00 | 13.61 | DIC |
| ATOM | 89 | N | ALA | 12 | 31.553 | 44.428 | 50.672 | 1.00 | 13.51 | DIC |
| ATOM | 90 | CA | ALA | 12 | 32.917 | 44.197 | 50.211 | 1.00 | 13.12 | DIC |
| ATOM | 91 | CB | ALA | 12 | 32.929 | 43.146 | 49.089 | 1.00 | 13.34 | DIC |
| ATOM | 92 | C | ALA | 12 | 33.604 | 45.484 | 49.736 | 1.00 | 11.96 | DIC |
| ATOM | 93 | O | ALA | 12 | 34.774 | 45.713 | 50.038 | 1.00 | 9.55 | DIC |
| ATOM | 94 | N | ALA | 13 | 32.888 | 46.325 | 48.995 | 1.00 | 11.10 | DIC |
| ATOM | 95 | CA | ALA | 13 | 33.491 | 47.565 | 48.499 | 1.00 | 11.93 | DIC |
| ATOM | 96 | CB | ALA | 13 | 32.550 | 48.251 | 47.499 | 1.00 | 10.97 | DIC |
| ATOM | 97 | C | ALA | 13 | 33.832 | 48.507 | 49.660 | 1.00 | 11.31 | DIC |
| ATOM | 98 | O | ALA | 13 | 34.914 | 49.097 | 49.700 | 1.00 | 12.37 | DIC |
| ATOM | 99 | N | TYR | 14 | 32.915 | 48.625 | 50.613 | 1.00 | 12.09 | DIC |
| ATOM | 100 | CA | TYR | 14 | 33.114 | 49.479 | 51.782 | 1.00 | 10.69 | DIC |
| ATOM | 101 | CB | TYR | 14 | 31.813 | 49.571 | 52.590 | 1.00 | 10.74 | DIC |
| ATOM | 102 | CG | TYR | 14 | 30.615 | 49.952 | 51.743 | 1.00 | 13.01 | DIC |
| ATOM | 103 | CD1 | TYR | 14 | 30.753 | 50.842 | 50.677 | 1.00 | 12.55 | DIC |
| ATOM | 104 | CE1 | TYR | 14 | 29.669 | 51.210 | 49.896 | 1.00 | 13.46 | DIC |
| ATOM | 105 | CD2 | TYR | 14 | 29.347 | 49.434 | 52.010 | 1.00 | 13.02 | DIC |
| ATOM | 106 | CE2 | TYR | 14 | 28.245 | 49.799 | 51.232 | 1.00 | 16.28 | DIC |
| ATOM | 107 | CZ | TYR | 14 | 28.424 | 50.693 | 50.176 | 1.00 | 16.05 | DIC |
| ATOM | 108 | OH | TYR | 14 | 27.358 | 51.101 | 49.416 | 1.00 | 19.24 | DIC |
| ATOM | 109 | C | TYR | 14 | 34.246 | 48.951 | 52.659 | 1.00 | 10.96 | DIC |
| ATOM | 110 | O | TYR | 14 | 35.013 | 49.730 | 53.237 | 1.00 | 10.92 | DIC |
| ATOM | 111 | N | LEU | 15 | 34.339 | 47.626 | 52.770 | 1.00 | 10.75 | DIC |
| ATOM | 112 | CA | LEU | 15 | 35.396 | 46.994 | 53.550 | 1.00 | 10.52 | DIC |
| ATOM | 113 | CB | LEU | 15 | 35.303 | 45.466 | 53.434 | 1.00 | 11.98 | DIC |
| ATOM | 114 | CG | LEU | 15 | 36.572 | 44.675 | 53.789 | 1.00 | 14.65 | DIC |
| ATOM | 115 | CD1 | LEU | 15 | 36.856 | 44.790 | 55.290 | 1.00 | 15.28 | DIC |
| ATOM | 116 | CD2 | LEU | 15 | 36.396 | 43.209 | 53.395 | 1.00 | 13.71 | DIC |
| ATOM | 117 | C | LEU | 15 | 36.751 | 47.458 | 53.010 | 1.00 | 10.20 | DIC |
| ATOM | 118 | O | LEU | 15 | 37.645 | 47.823 | 53.771 | 1.00 | 10.38 | DIC |
| ATOM | 119 | N | CYS | 16 | 36.896 | 47.424 | 51.686 | 1.00 | 12.12 | DIC |
| ATOM | 120 | CA | CYS | 16 | 38.136 | 47.838 | 51.038 | 1.00 | 11.09 | DIC |
| ATOM | 121 | CB | CYS | 16 | 37.997 | 47.725 | 49.515 | 1.00 | 12.05 | DIC |
| ATOM | 122 | SG | CYS | 16 | 39.517 | 48.138 | 48.641 | 1.00 | 12.15 | DIC |
| ATOM | 123 | C | CYS | 16 | 38.519 | 49.274 | 51.415 | 1.00 | 10.93 | DIC |
| ATOM | 124 | O | CYS | 16 | 39.671 | 49.550 | 51.757 | 1.00 | 11.30 | DIC |
| ATOM | 125 | N | VAL | 17 | 37.553 | 50.185 | 51.353 | 1.00 | 10.79 | DIC |
| ATOM | 126 | CA | VAL | 17 | 37.805 | 51.585 | 51.695 | 1.00 | 12.24 | DIC |
| ATOM | 127 | CB | VAL | 17 | 36.549 | 52.448 | 51.438 | 1.00 | 12.01 | DIC |
| ATOM | 128 | CG1 | VAL | 17 | 36.734 | 53.840 | 52.022 | 1.00 | 11.58 | DIC |
| ATOM | 129 | CG2 | VAL | 17 | 36.282 | 52.531 | 49.929 | 1.00 | 12.39 | DIC |
| ATOM | 130 | C | VAL | 17 | 38.231 | 51.720 | 53.159 | 1.00 | 12.59 | DIC |
| ATOM | 131 | O | VAL | 17 | 39.192 | 52.422 | 53.478 | 1.00 | 14.20 | DIC |
| ATOM | 132 | N | ALA | 18 | 37.517 | 51.038 | 54.045 | 1.00 | 12.29 | DIC |
| ATOM | 133 | CA | ALA | 18 | 37.831 | 51.084 | 55.470 | 1.00 | 11.27 | DIC |
| ATOM | 134 | CB | ALA | 18 | 36.816 | 50.262 | 56.247 | 1.00 | 11.41 | DIC |
| ATOM | 135 | C | ALA | 18 | 39.237 | 50.539 | 55.723 | 1.00 | 12.12 | DIC |
| ATOM | 136 | O | ALA | 18 | 40.018 | 51.123 | 56.479 | 1.00 | 9.33 | DIC |
| ATOM | 137 | N | ALA | 19 | 39.545 | 49.416 | 55.081 | 1.00 | 11.56 | DIC |
| ATOM | 138 | CA | ALA | 19 | 40.840 | 48.772 | 55.234 | 1.00 | 12.42 | DIC |
| ATOM | 139 | CB | ALA | 19 | 40.883 | 47.481 | 54.418 | 1.00 | 12.78 | DIC |
| ATOM | 140 | C | ALA | 19 | 41.966 | 49.705 | 54.806 | 1.00 | 13.64 | DIC |
| ATOM | 141 | O | ALA | 19 | 42.970 | 49.834 | 55.507 | 1.00 | 12.89 | DIC |
| ATOM | 142 | N | LYS | 20 | 41.805 | 50.362 | 53.661 | 1.00 | 14.08 | DIC |
| ATOM | 143 | CA | LYS | 20 | 42.836 | 51.279 | 53.202 | 1.00 | 14.66 | DIC |
| ATOM | 144 | CB | LYS | 20 | 42.532 | 51.789 | 51.793 | 1.00 | 15.85 | DIC |
| ATOM | 145 | CG | LYS | 20 | 43.694 | 52.575 | 51.192 | 1.00 | 20.09 | DIC |
| ATOM | 146 | CD | LYS | 20 | 43.502 | 52.849 | 49.706 | 1.00 | 22.70 | DIC |
| ATOM | 147 | CE | LYS | 20 | 44.681 | 53.638 | 49.161 | 1.00 | 22.43 | DIC |
| ATOM | 148 | NZ | LYS | 20 | 44.540 | 53.943 | 47.712 | 1.00 | 24.73 | DIC |
| ATOM | 149 | C | LYS | 20 | 42.993 | 52.458 | 54.162 | 1.00 | 14.30 | DIC |
| ATOM | 150 | O | LYS | 20 | 44.098 | 52.980 | 54.329 | 1.00 | 13.09 | DIC |
| ATOM | 151 | N | SER | 21 | 41.906 | 52.883 | 54.809 | 1.00 | 13.25 | DIC |
| ATOM | 152 | CA | SER | 21 | 42.033 | 54.003 | 55.741 | 1.00 | 12.72 | DIC |
| ATOM | 153 | CB | SER | 21 | 40.660 | 54.440 | 56.283 | 1.00 | 12.54 | DIC |
| ATOM | 154 | OG | SER | 21 | 40.244 | 53.667 | 57.395 | 1.00 | 12.59 | DIC |
| ATOM | 155 | C | SER | 21 | 42.962 | 53.586 | 56.886 | 1.00 | 12.00 | DIC |
| ATOM | 156 | O | SER | 21 | 43.748 | 54.387 | 57.392 | 1.00 | 13.07 | DIC |
| ATOM | 157 | N | VAL | 22 | 42.890 | 52.319 | 57.279 | 1.00 | 11.25 | DIC |
| ATOM | 158 | CA | VAL | 22 | 43.747 | 51.816 | 58.348 | 1.00 | 11.30 | DIC |
| ATOM | 159 | CB | VAL | 22 | 43.360 | 50.374 | 58.752 | 1.00 | 11.76 | DIC |
| ATOM | 160 | CG1 | VAL | 22 | 44.298 | 49.870 | 59.821 | 1.00 | 11.30 | DIC |
| ATOM | 161 | CG2 | VAL | 22 | 41.921 | 50.339 | 59.241 | 1.00 | 11.48 | DIC |
| ATOM | 162 | C | VAL | 22 | 45.212 | 51.814 | 57.912 | 1.00 | 11.89 | DIC |
| ATOM | 163 | O | VAL | 22 | 46.095 | 52.246 | 58.662 | 1.00 | 12.69 | DIC |
| ATOM | 164 | N | GLU | 23 | 45.478 | 51.328 | 56.704 | 1.00 | 10.93 | DIC |
| ATOM | 165 | CA | GLU | 23 | 46.854 | 51.295 | 56.219 | 1.00 | 13.03 | DIC |
| ATOM | 166 | CB | GLU | 23 | 46.952 | 50.615 | 54.849 | 1.00 | 14.30 | DIC |
| ATOM | 167 | CG | GLU | 23 | 46.544 | 49.160 | 54.823 | 1.00 | 14.62 | DIC |
| ATOM | 168 | CD | GLU | 23 | 46.836 | 48.512 | 53.480 | 1.00 | 16.61 | DIC |
| ATOM | 169 | OE1 | GLU | 23 | 46.678 | 49.196 | 52.449 | 1.00 | 17.40 | DIC |
| ATOM | 170 | OE2 | GLU | 23 | 47.209 | 47.317 | 53.453 | 1.00 | 16.65 | DIC |
| ATOM | 171 | C | GLU | 23 | 47.407 | 52.707 | 56.104 | 1.00 | 12.51 | DIC |
| ATOM | 172 | O | GLU | 23 | 48.538 | 52.969 | 56.502 | 1.00 | 14.38 | DIC |
| ATOM | 173 | N | ALA | 24 | 46.603 | 53.620 | 55.572 | 1.00 | 11.76 | DIC |
| ATOM | 174 | CA | ALA | 24 | 47.046 | 54.997 | 55.397 | 1.00 | 12.06 | DIC |
| ATOM | 175 | CB | ALA | 24 | 45.945 | 55.827 | 54.716 | 1.00 | 11.87 | DIC |
| ATOM | 176 | C | ALA | 24 | 47.445 | 55.635 | 56.722 | 1.00 | 11.80 | DIC |
| ATOM | 177 | O | ALA | 24 | 48.393 | 56.425 | 56.775 | 1.00 | 11.54 | DIC |
| ATOM | 178 | N | ALA | 25 | 46.734 | 55.279 | 57.789 | 1.00 | 11.43 | DIC |
| ATOM | 179 | CA | ALA | 25 | 47.005 | 55.825 | 59.123 | 1.00 | 11.35 | DIC |
| ATOM | 180 | CB | ALA | 25 | 45.757 | 55.698 | 59.998 | 1.00 | 11.07 | DIC |
| ATOM | 181 | C | ALA | 25 | 48.194 | 55.180 | 59.836 | 1.00 | 11.54 | DIC |
| ATOM | 182 | O | ALA | 25 | 48.596 | 55.630 | 60.904 | 1.00 | 10.97 | DIC |
| ATOM | 183 | N | HIS | 26 | 48.753 | 54.123 | 59.264 | 1.00 | 11.65 | DIC |
| ATOM | 184 | CA | HIS | 26 | 49.899 | 53.464 | 59.893 | 1.00 | 13.56 | DIC |
| ATOM | 185 | CB | HIS | 26 | 49.452 | 52.198 | 60.628 | 1.00 | 13.72 | DIC |
| ATOM | 186 | CG | HIS | 26 | 48.350 | 52.425 | 61.617 | 1.00 | 14.14 | DIC |
| ATOM | 187 | CD2 | HIS | 26 | 48.381 | 52.626 | 62.958 | 1.00 | 13.28 | DIC |
| ATOM | 188 | ND1 | HIS | 26 | 47.020 | 52.455 | 61.255 | 1.00 | 11.89 | DIC |
| ATOM | 189 | CE1 | HIS | 26 | 46.277 | 52.660 | 62.330 | 1.00 | 12.65 | DIC |
| ATOM | 190 | NE2 | HIS | 26 | 47.078 | 52.768 | 63.375 | 1.00 | 14.11 | DIC |
| ATOM | 191 | C | HIS | 26 | 50.940 | 53.111 | 58.833 | 1.00 | 13.66 | DIC |
| ATOM | 192 | O | HIS | 26 | 51.151 | 51.941 | 58.512 | 1.00 | 13.39 | DIC |
| ATOM | 193 | N | PRO | 27 | 51.615 | 54.132 | 58.284 | 1.00 | 14.55 | DIC |
| ATOM | 194 | CD | PRO | 27 | 51.529 | 55.536 | 58.728 | 1.00 | 15.30 | DIC |
| ATOM | 195 | CA | PRO | 27 | 52.640 | 53.966 | 57.247 | 1.00 | 14.85 | DIC |
| ATOM | 196 | CB | PRO | 27 | 53.078 | 55.408 | 56.957 | 1.00 | 16.08 | DIC |
| ATOM | 197 | CG | PRO | 27 | 52.856 | 56.104 | 58.270 | 1.00 | 17.10 | DIC |
| ATOM | 198 | C | PRO | 27 | 53.810 | 53.046 | 57.592 | 1.00 | 15.06 | DIC |
| ATOM | 199 | O | PRO | 27 | 54.397 | 52.425 | 56.696 | 1.00 | 15.60 | DIC |
| ATOM | 200 | N | ASP | 28 | 54.149 | 52.936 | 58.874 | 1.00 | 13.42 | DIC |
| ATOM | 201 | CA | ASP | 28 | 55.265 | 52.074 | 59.251 | 1.00 | 13.91 | DIC |
| ATOM | 202 | CB | ASP | 28 | 56.401 | 52.896 | 59.867 | 1.00 | 12.65 | DIC |
| ATOM | 203 | CG | ASP | 28 | 57.698 | 52.099 | 59.970 | 1.00 | 13.98 | DIC |
| ATOM | 204 | OD1 | ASP | 28 | 58.008 | 51.345 | 59.017 | 1.00 | 14.45 | DIC |
| ATOM | 205 | OD2 | ASP | 28 | 58.405 | 52.229 | 60.988 | 1.00 | 11.69 | DIC |
| ATOM | 206 | C | ASP | 28 | 54.879 | 50.946 | 60.197 | 1.00 | 13.78 | DIC |
| ATOM | 207 | O | ASP | 28 | 55.708 | 50.445 | 60.950 | 1.00 | 13.88 | DIC |
| ATOM | 208 | N | THR | 29 | 53.617 | 50.546 | 60.160 | 1.00 | 14.27 | DIC |
| ATOM | 209 | CA | THR | 29 | 53.166 | 49.457 | 61.011 | 1.00 | 15.21 | DIC |
| ATOM | 210 | CB | THR | 29 | 52.082 | 49.917 | 62.001 | 1.00 | 15.30 | DIC |
| ATOM | 211 | OG1 | THR | 29 | 52.567 | 51.025 | 62.772 | 1.00 | 15.73 | DIC |
| ATOM | 212 | CG2 | THR | 29 | 51.732 | 48.782 | 62.948 | 1.00 | 17.03 | DIC |
| ATOM | 213 | C | THR | 29 | 52.594 | 48.339 | 60.149 | 1.00 | 15.61 | DIC |
| ATOM | 214 | O | THR | 29 | 51.884 | 48.596 | 59.176 | 1.00 | 14.30 | DIC |
| ATOM | 215 | N | GLU | 30 | 52.927 | 47.101 | 60.499 | 1.00 | 16.81 | DIC |
| ATOM | 216 | CA | GLU | 30 | 52.427 | 45.936 | 59.779 | 1.00 | 17.79 | DIC |
| ATOM | 217 | CB | GLU | 30 | 53.187 | 44.680 | 60.215 | 1.00 | 21.66 | DIC |
| ATOM | 218 | CG | GLU | 30 | 53.405 | 43.645 | 59.115 | 1.00 | 28.54 | DIC |
| ATOM | 219 | CD | GLU | 30 | 54.135 | 44.217 | 57.900 | 1.00 | 29.87 | DIC |
| ATOM | 220 | OE1 | GLU | 30 | 53.472 | 44.811 | 57.024 | 1.00 | 31.57 | DIC |
| ATOM | 221 | OE2 | GLU | 30 | 55.375 | 44.084 | 57.826 | 1.00 | 33.06 | DIC |
| ATOM | 222 | C | GLU | 30 | 50.957 | 45.790 | 60.147 | 1.00 | 16.87 | DIC |
| ATOM | 223 | O | GLU | 30 | 50.617 | 45.687 | 61.325 | 1.00 | 17.37 | DIC |
| ATOM | 224 | N | ILE | 31 | 50.082 | 45.800 | 59.151 | 1.00 | 15.55 | DIC |
| ATOM | 225 | CA | ILE | 31 | 48.658 | 45.652 | 59.418 | 1.00 | 15.24 | DIC |
| ATOM | 226 | CB | ILE | 31 | 47.821 | 46.802 | 58.782 | 1.00 | 14.65 | DIC |
| ATOM | 227 | CG2 | ILE | 31 | 46.341 | 46.638 | 59.150 | 1.00 | 15.64 | DIC |
| ATOM | 228 | CG1 | ILE | 31 | 48.323 | 48.163 | 59.278 | 1.00 | 14.25 | DIC |
| ATOM | 229 | CD | ILE | 31 | 48.159 | 48.389 | 60.765 | 1.00 | 15.14 | DIC |
| ATOM | 230 | C | ILE | 31 | 48.188 | 44.319 | 58.849 | 1.00 | 14.29 | DIC |
| ATOM | 231 | O | ILE | 31 | 48.129 | 44.132 | 57.634 | 1.00 | 14.29 | DIC |
| ATOM | 232 | N | ARG | 32 | 47.877 | 43.386 | 59.739 | 1.00 | 13.00 | DIC |
| ATOM | 233 | CA | ARG | 32 | 47.398 | 42.081 | 59.328 | 1.00 | 12.73 | DIC |
| ATOM | 234 | CB | ARG | 32 | 48.045 | 40.984 | 60.182 | 1.00 | 15.39 | DIC |
| ATOM | 235 | CG | ARG | 32 | 49.580 | 40.988 | 60.101 | 1.00 | 21.06 | DIC |
| ATOM | 236 | CD | ARG | 32 | 50.221 | 39.798 | 60.809 | 1.00 | 25.85 | DIC |
| ATOM | 237 | NE | ARG | 32 | 49.830 | 39.700 | 62.216 | 1.00 | 31.82 | DIC |
| ATOM | 238 | CZ | ARG | 32 | 50.327 | 38.808 | 63.069 | 1.00 | 35.29 | DIC |
| ATOM | 239 | NH1 | ARG | 32 | 51.246 | 37.938 | 62.657 | 1.00 | 36.77 | DIC |
| ATOM | 240 | NH2 | ARG | 32 | 49.897 | 38.772 | 64.329 | 1.00 | 34.04 | DIC |
| ATOM | 241 | C | ARG | 32 | 45.884 | 42.079 | 59.487 | 1.00 | 11.59 | DIC |
| ATOM | 242 | O | ARG | 32 | 45.365 | 42.130 | 60.604 | 1.00 | 10.59 | DIC |
| ATOM | 243 | N | PHE | 33 | 45.190 | 42.061 | 58.353 | 1.00 | 9.57 | DIC |
| ATOM | 244 | CA | PHE | 33 | 43.733 | 42.059 | 58.314 | 1.00 | 11.07 | DIC |
| ATOM | 245 | CB | PHE | 33 | 43.233 | 42.709 | 57.021 | 1.00 | 9.65 | DIC |
| ATOM | 246 | CG | PHE | 33 | 43.473 | 44.187 | 56.942 | 1.00 | 9.60 | DIC |
| ATOM | 247 | CD1 | PHE | 33 | 42.751 | 45.069 | 57.741 | 1.00 | 8.83 | DIC |
| ATOM | 248 | CD2 | PHE | 33 | 44.394 | 44.703 | 56.034 | 1.00 | 10.43 | DIC |
| ATOM | 249 | CE1 | PHE | 33 | 42.939 | 46.449 | 57.634 | 1.00 | 9.43 | DIC. |
| ATOM | 250 | CE2 | PHE | 33 | 44.590 | 46.078 | 55.918 | 1.00 | 9.57 | DIC |
| ATOM | 251 | CZ | PHE | 33 | 43.861 | 46.953 | 56.718 | 1.00 | 9.32 | DIC |
| ATOM | 252 | C | PHE | 33 | 43.142 | 40.652 | 58.391 | 1.00 | 11.46 | DIC |
| ATOM | 253 | O | PHE | 33 | 43.577 | 39.741 | 57.678 | 1.00 | 12.46 | DIC |
| ATOM | 254 | N | HIS | 34 | 42.138 | 40.495 | 59.246 | 1.00 | 10.60 | DIC |
| ATOM | 255 | CA | HIS | 34 | 41.437 | 39.222 | 59.414 | 1.00 | 12.40 | DIC |
| ATOM | 256 | CB | HIS | 34 | 41.567 | 38.728 | 60.859 | 1.00 | 11.10 | DIC |
| ATOM | 257 | CG | HIS | 34 | 42.983 | 38.591 | 61.328 | 1.00 | 12.97 | DIC |
| ATOM | 258 | CD2 | HIS | 34 | 43.844 | 39.503 | 61.841 | 1.00 | 12.88 | DIC |
| ATOM | 259 | ND1 | HIS | 34 | 43.667 | 37.394 | 61.300 | 1.00 | 14.47 | DIC |
| ATOM | 260 | CE1 | HIS | 34 | 44.887 | 37.574 | 61.776 | 1.00 | 14.13 | DIC |
| ATOM | 261 | NE2 | HIS | 34 | 45.019 | 38.845 | 62.113 | 1.00 | 14.30 | DIC |
| ATOM | 262 | C | HIS | 34 | 39.978 | 39.545 | 59.103 | 1.00 | 12.39 | DIC |
| ATOM | 263 | O | HIS | 34 | 39.337 | 40.294 | 59.840 | 1.00 | 12.46 | DIC |
| ATOM | 264 | N | VAL | 35 | 39.449 | 38.987 | 58.019 | 1.00 | 10.81 | DIC |
| ATOM | 265 | CA | VAL | 35 | 38.073 | 39.280 | 57.643 | 1.00 | 10.52 | DIC |
| ATOM | 266 | CB | VAL | 35 | 37.991 | 39.696 | 56.150 | 1.00 | 11.67 | DIC |
| ATOM | 267 | CG1 | VAL | 35 | 36.571 | 40.115 | 55.793 | 1.00 | 12.07 | DIC |
| ATOM | 268 | CG2 | VAL | 35 | 38.970 | 40.838 | 55.875 | 1.00 | 11.00 | DIC |
| ATOM | 269 | C | VAL | 35 | 37.088 | 38.141 | 57.878 | 1.00 | 11.06 | DIC |
| ATOM | 270 | O | VAL | 35 | 37.229 | 37.055 | 57.307 | 1.00 | 10.52 | DIC |
| ATOM | 271 | N | LEU | 36 | 36.096 | 38.391 | 58.730 | 1.00 | 10.89 | DIC |
| ATOM | 272 | CA | LEU | 36 | 35.057 | 37.401 | 58.994 | 1.00 | 11.46 | DIC |
| ATOM | 273 | CB | LEU | 36 | 34.272 | 37.769 | 60.262 | 1.00 | 11.18 | DIC |
| ATOM | 274 | CG | LEU | 36 | 35.134 | 37.949 | 61.527 | 1.00 | 11.27 | DIC |
| ATOM | 275 | CD1 | LEU | 36 | 34.224 | 38.089 | 62.748 | 1.00 | 10.72 | DIC |
| ATOM | 276 | CD2 | LEU | 36 | 36.061 | 36.750 | 61.721 | 1.00 | 9.09 | DIC |
| ATOM | 277 | C | LEU | 36 | 34.201 | 37.528 | 57.732 | 1.00 | 12.89 | DIC |
| ATOM | 278 | O | LEU | 36 | 33.465 | 38.501 | 57.556 | 1.00 | 11.93 | DIC |
| ATOM | 279 | N | ASP | 37 | 34.351 | 36.547 | 56.845 | 1.00 | 14.29 | DIC |
| ATOM | 280 | CA | ASP | 37 | 33.694 | 36.513 | 55.538 | 1.00 | 14.87 | DIC |
| ATOM | 281 | CB | ASP | 37 | 34.654 | 35.854 | 54.538 | 1.00 | 17.19 | DIC |
| ATOM | 282 | CG | ASP | 37 | 34.122 | 35.845 | 53.120 | 1.00 | 15.95 | DIC |
| ATOM | 283 | OD1 | ASP | 37 | 32.917 | 36.093 | 52.917 | 1.00 | 16.13 | DIC |
| ATOM | 284 | OD2 | ASP | 37 | 34.920 | 35.571 | 52.207 | 1.00 | 19.63 | DIC |
| ATOM | 285 | C | ASP | 37 | 32.362 | 35.774 | 55.545 | 1.00 | 16.83 | DIC |
| ATOM | 286 | O | ASP | 37 | 32.320 | 34.539 | 55.625 | 1.00 | 14.89 | DIC |
| ATOM | 287 | N | ALA | 38 | 31.275 | 36.531 | 55.429 | 1.00 | 15.59 | DIC |
| ATOM | 288 | CA | ALA | 38 | 29.947 | 35.941 | 55.447 | 1.00 | 18.57 | DIC |
| ATOM | 289 | CB | ALA | 38 | 29.010 | 36.795 | 56.307 | 1.00 | 19.77 | DIC |
| ATOM | 290 | C | ALA | 38 | 29.330 | 35.719 | 54.069 | 1.00 | 19.75 | DIC |
| ATOM | 291 | O | ALA | 38 | 28.111 | 35.689 | 53.937 | 1.00 | 23.40 | DIC |
| ATOM | 292 | N | GLY | 39 | 30.161 | 35.560 | 53.045 | 1.00 | 19.13 | DIC |
| ATOM | 293 | CA | GLY | 39 | 29.627 | 35.321 | 51.717 | 1.00 | 17.91 | DIC |
| ATOM | 294 | C | GLY | 39 | 30.163 | 36.247 | 50.641 | 1.00 | 16.51 | DIC |
| ATOM | 295 | O | GLY | 39 | 29.486 | 36.494 | 49.649 | 1.00 | 18.04 | DIC |
| ATOM | 296 | N | ILE | 40 | 31.371 | 36.768 | 50.832 | 1.00 | 14.45 | DIC |
| ATOM | 297 | CA | ILE | 40 | 31.975 | 37.662 | 49.848 | 1.00 | 14.34 | DIC |
| ATOM | 298 | CB | ILE | 40 | 33.195 | 38.400 | 50.450 | 1.00 | 13.99 | DIC |
| ATOM | 299 | CG2 | ILE | 40 | 33.718 | 39.424 | 49.460 | 1.00 | 13.81 | DIC |
| ATOM | 300 | CG1 | ILE | 40 | 32.788 | 39.096 | 51.760 | 1.00 | 13.39 | DIC |
| ATOM | 301 | CD | ILE | 40 | 33.946 | 39.762 | 52.517 | 1.00 | 13.39 | DIC |
| ATOM | 302 | C | ILE | 40 | 32.407 | 36.846 | 48.621 | 1.00 | 14.46 | DIC |
| ATOM | 303 | O | ILE | 40 | 33.064 | 35.817 | 48.751 | 1.00 | 14.76 | DIC |
| ATOM | 304 | N | SER | 41 | 32.029 | 37.299 | 47.429 | 1.00 | 14.97 | DIC |
| ATOM | 305 | CA | SER | 41 | 32.376 | 36.573 | 46.207 | 1.00 | 15.36 | DIC |
| ATOM | 306 | CB | SER | 41 | 31.711 | 37.221 | 44.987 | 1.00 | 15.16 | DIC |
| ATOM | 307 | OG | SER | 41 | 32.356 | 38.436 | 44.642 | 1.00 | 14.14 | DIC |
| ATOM | 308 | C | SER | 41 | 33.880 | 36.531 | 45.988 | 1.00 | 16.18 | DIC |
| ATOM | 309 | O | SER | 41 | 34.615 | 37.374 | 46.504 | 1.00 | 17.61 | DIC |
| ATOM | 310 | N | GLU | 42 | 34.334 | 35.546 | 45.219 | 1.00 | 16.89 | DIC |
| ATOM | 311 | CA | GLU | 42 | 35.754 | 35.406 | 44.922 | 1.00 | 16.48 | DIC |
| ATOM | 312 | CB | GLU | 42 | 36.008 | 34.191 | 44.018 | 1.00 | 18.56 | DIC |
| ATOM | 313 | CG | GLU | 42 | 37.468 | 34.005 | 43.605 | 1.00 | 20.32 | DIC |
| ATOM | 314 | CD | GLU | 42 | 37.919 | 34.923 | 42.467 | 1.00 | 24.54 | DIC |
| ATOM | 315 | OE1 | GLU | 42 | 39.149 | 35.117 | 42.326 | 1.00 | 27.22 | DIC |
| ATOM | 316 | OE2 | GLU | 42 | 37.064 | 35.440 | 41.707 | 1.00 | 24.68 | DIC |
| ATOM | 317 | C | GLU | 42 | 36.249 | 36.654 | 44.217 | 1.00 | 15.88 | DIC |
| ATOM | 318 | O | GLU | 42 | 37.381 | 37.083 | 44.423 | 1.00 | 14.39 | DIC |
| ATOM | 319 | N | ALA | 43 | 35.399 | 37.234 | 43.378 | 1.00 | 17.03 | DIC |
| ATOM | 320 | CA | ALA | 43 | 35.792 | 38.427 | 42.640 | 1.00 | 17.82 | DIC |
| ATOM | 321 | CB | ALA | 43 | 34.752 | 38.758 | 41.562 | 1.00 | 18.48 | DIC |
| ATOM | 322 | C | ALA | 43 | 35.966 | 39.605 | 43.587 | 1.00 | 16.49 | DIC |
| ATOM | 323 | O | ALA | 43 | 36.898 | 40.396 | 43.440 | 1.00 | 15.18 | DIC |
| ATOM | 324 | N | ASN | 44 | 35.086 | 39.717 | 44.574 | 1.00 | 15.20 | DIC |
| ATOM | 325 | CA | ASN | 44 | 35.200 | 40.836 | 45.500 | 1.00 | 16.12 | DIC |
| ATOM | 326 | CB | ASN | 44 | 33.880 | 41.055 | 46.248 | 1.00 | 15.18 | DIC |
| ATOM | 327 | CG | ASN | 44 | 32.838 | 41.754 | 45.385 | 1.00 | 19.28 | DIC |
| ATOM | 328 | OD1 | ASN | 44 | 33.171 | 42.636 | 44.586 | 1.00 | 19.46 | DIC |
| ATOM | 329 | ND2 | ASN | 44 | 31.571 | 41.378 | 45.551 | 1.00 | 18.90 | DIC |
| ATOM | 330 | C | ASN | 44 | 36.372 | 40.684 | 46.470 | 1.00 | 15.26 | DIC |
| ATOM | 331 | O | ASN | 44 | 36.961 | 41.673 | 46.888 | 1.00 | 15.20 | DIC |
| ATOM | 332 | N | ARG | 45 | 36.724 | 39.449 | 46.817 | 1.00 | 16.07 | DIC |
| ATOM | 333 | CA | ARG | 45 | 37.851 | 39.234 | 47.712 | 1.00 | 16.99 | DIC |
| ATOM | 334 | CB | ARG | 45 | 37.909 | 37.769 | 48.167 | 1.00 | 19.65 | DIC |
| ATOM | 335 | CG | ARG | 45 | 36.581 | 37.274 | 48.728 | 1.00 | 24.95 | DIC |
| ATOM | 336 | CD | ARG | 45 | 36.731 | 36.329 | 49.909 | 1.00 | 30.41 | DIC |
| ATOM | 337 | NE | ARG | 45 | 37.494 | 35.125 | 49.592 | 1.00 | 34.93 | DIC |
| ATOM | 338 | CZ | ARG | 45 | 37.562 | 34.059 | 50.387 | 1.00 | 38.05 | DIC |
| ATOM | 339 | NH1 | ARG | 45 | 36.907 | 34.046 | 51.542 | 1.00 | 39.62 | DIC |
| ATOM | 340 | NH2 | ARG | 45 | 38.294 | 33.007 | 50.037 | 1.00 | 39.66 | DIC |
| ATOM | 341 | C | ARG | 45 | 39.128 | 39.620 | 46.963 | 1.00 | 16.64 | DIC |
| ATOM | 342 | O | ARG | 45 | 40.009 | 40.267 | 47.522 | 1.00 | 15.53 | DIC |
| ATOM | 343 | N | ALA | 46 | 39.218 | 39.242 | 45.690 | 1.00 | 14.76 | DIC |
| ATOM | 344 | CA | ALA | 46 | 40.396 | 39.582 | 44.901 | 1.00 | 15.52 | DIC |
| ATOM | 345 | CB | ALA | 46 | 40.321 | 38.933 | 43.514 | 1.00 | 16.34 | DIC |
| ATOM | 346 | C | ALA | 46 | 40.499 | 41.099 | 44.763 | 1.00 | 15.93 | DIC |
| ATOM | 347 | O | ALA | 46 | 41.581 | 41.669 | 44.890 | 1.00 | 16.56 | DIC |
| ATOM | 348 | N | ALA | 47 | 39.367 | 41.753 | 44.514 | 1.00 | 15.64 | DIC |
| ATOM | 349 | CA | ALA | 47 | 39.358 | 43.204 | 44.355 | 1.00 | 15.26 | DIC |
| ATOM | 350 | CB | ALA | 47 | 37.985 | 43.671 | 43.903 | 1.00 | 14.76 | DIC |
| ATOM | 351 | C | ALA | 47 | 39.762 | 43.918 | 45.643 | 1.00 | 14.97 | DIC |
| ATOM | 352 | O | ALA | 47 | 40.498 | 44.897 | 45.612 | 1.00 | 15.32 | DIC |
| ATOM | 353 | N | VAL | 48 | 39.274 | 43.435 | 46.781 | 1.00 | 14.13 | DIC |
| ATOM | 354 | CA | VAL | 48 | 39.632 | 44.054 | 48.048 | 1.00 | 12.62 | DIC |
| ATOM | 355 | CB | VAL | 48 | 38.887 | 43.393 | 49.229 | 1.00 | 11.63 | DIC |
| ATOM | 356 | CG1 | VAL | 48 | 39.427 | 43.922 | 50.543 | 1.00 | 10.65 | DIC |
| ATOM | 357 | CG2 | VAL | 48 | 37.391 | 43.675 | 49.127 | 1.00 | 10.00 | DIC |
| ATOM | 358 | C | VAL | 48 | 41.142 | 43.927 | 48.271 | 1.00 | 13.29 | DIC |
| ATOM | 359 | O | VAL | 48 | 41.810 | 44.899 | 48.612 | 1.00 | 12.89 | DIC |
| ATOM | 360 | N | ALA | 49 | 41.676 | 42.726 | 48.062 | 1.00 | 13.20 | DIC |
| ATOM | 361 | CA | ALA | 49 | 43.099 | 42.476 | 48.257 | 1.00 | 15.72 | DIC |
| ATOM | 362 | CB | ALA | 49 | 43.389 | 40.974 | 48.140 | 1.00 | 14.62 | DIC |
| ATOM | 363 | C | ALA | 49 | 43.977 | 43.255 | 47.282 | 1.00 | 17.17 | DIC |
| ATOM | 364 | O | ALA | 49 | 45.025 | 43.776 | 47.666 | 1.00 | 18.51 | DIC |
| ATOM | 365 | N | ALA | 50 | 43.549 | 43.342 | 46.026 | 1.00 | 17.97 | DIC |
| ATOM | 366 | CA | ALA | 50 | 44.321 | 44.057 | 45.012 | 1.00 | 19.51 | DIC |
| ATOM | 367 | CB | ALA | 50 | 43.651 | 43.915 | 43.643 | 1.00 | 19.91 | DIC |
| ATOM | 368 | C | ALA | 50 | 44.514 | 45.539 | 45.335 | 1.00 | 19.92 | DIC |
| ATOM | 369 | O | ALA | 50 | 45.414 | 46.178 | 44.796 | 1.00 | 20.21 | DIC |
| ATOM | 370 | N | ASN | 51 | 43.666 | 46.089 | 46.200 | 1.00 | 19.48 | DIC |
| ATOM | 371 | CA | ASN | 51 | 43.770 | 47.505 | 46.565 | 1.00 | 18.23 | DIC |
| ATOM | 372 | CB | ASN | 51 | 42.379 | 48.125 | 46.722 | 1.00 | 16.61 | DIC |
| ATOM | 373 | CG | ASN | 51 | 41.690 | 48.369 | 45.391 | 1.00 | 17.51 | DIC |
| ATOM | 374 | OD1 | ASN | 51 | 42.160 | 49.161 | 44.577 | 1.00 | 18.25 | DIC |
| ATOM | 375 | ND2 | ASN | 51 | 40.570 | 47.689 | 45.165 | 1.00 | 15.70 | DIC |
| ATOM | 376 | C | ASN | 51 | 44.551 | 47.733 | 47.855 | 1.00 | 18.98 | DIC |
| ATOM | 377 | O | ASN | 51 | 44.802 | 48.877 | 48.241 | 1.00 | 18.73 | DIC |
| ATOM | 378 | N | LEU | 52 | 44.929 | 46.657 | 48.533 | 1.00 | 18.72 | DIC |
| ATOM | 379 | CA | LEU | 52 | 45.671 | 46.812 | 49.778 | 1.00 | 20.64 | DIC |
| ATOM | 380 | CB | LEU | 52 | 45.208 | 45.778 | 50.805 | 1.00 | 18.56 | DIC |
| ATOM | 381 | CG | LEU | 52 | 43.701 | 45.819 | 51.069 | 1.00 | 18.19 | DIC |
| ATOM | 382 | CD1 | LEU | 52 | 43.336 | 44.772 | 52.106 | 1.00 | 19.78 | DIC |
| ATOM | 383 | CD2 | LEU | 52 | 43.292 | 47.202 | 51.529 | 1.00 | 18.99 | DIC |
| ATOM | 384 | C | LEU | 52 | 47.171 | 46.702 | 49.554 | 1.00 | 22.97 | DIC |
| ATOM | 385 | O | LEU | 52 | 47.626 | 46.310 | 48.476 | 1.00 | 21.94 | DIC |
| ATOM | 386 | N | ARG | 53 | 47.931 | 47.057 | 50.583 | 1.00 | 24.82 | DIC |
| ATOM | 387 | CA | ARG | 53 | 49.387 | 47.024 | 50.523 | 1.00 | 28.21 | DIC |
| ATOM | 388 | CB | ARG | 53 | 49.966 | 47.479 | 51.878 | 1.00 | 27.92 | DIC |
| ATOM | 389 | CG | ARG | 53 | 50.738 | 48.796 | 51.811 | 1.00 | 26.01 | DIC |
| ATOM | 390 | CD | ARG | 53 | 50.572 | 49.669 | 53.069 | 1.00 | 26.38 | DIC |
| ATOM | 391 | NE | ARG | 53 | 50.831 | 48.945 | 54.310 | 1.00 | 21.46 | DIC |
| ATOM | 392 | CZ | ARG | 53 | 50.875 | 49.507 | 55.516 | 1.00 | 22.17 | DIC |
| ATOM | 393 | NH1 | ARG | 53 | 50.682 | 50.814 | 55.662 | 1.00 | 22.29 | DIC |
| ATOM | 394 | NH2 | ARG | 53 | 51.098 | 48.757 | 56.585 | 1.00 | 18.63 | DIC |
| ATOM | 395 | C | ARG | 53 | 49.931 | 45.650 | 50.136 | 1.00 | 29.98 | DIC |
| ATOM | 396 | O | ARG | 53 | 49.397 | 44.613 | 50.534 | 1.00 | 30.96 | DIC |
| ATOM | 397 | N | GLY | 54 | 50.989 | 45.655 | 49.333 | 1.00 | 32.74 | DIC |
| ATOM | 398 | CA | GLY | 54 | 51.606 | 44.414 | 48.903 | 1.00 | 34.79 | DIC |
| ATOM | 399 | C | GLY | 54 | 50.684 | 43.486 | 48.136 | 1.00 | 37.38 | DIC |
| ATOM | 400 | O | GLY | 54 | 50.848 | 42.263 | 48.190 | 1.00 | 37.61 | DIC |
| ATOM | 401 | N | GLY | 55 | 49.718 | 44.061 | 47.421 | 1.00 | 37.91 | DIC |
| ATOM | 402 | CA | GLY | 55 | 48.782 | 43.259 | 46.654 | 1.00 | 38.40 | DIC |
| ATOM | 403 | C | GLY | 55 | 47.822 | 42.468 | 47.526 | 1.00 | 38.63 | DIC |
| ATOM | 404 | O | GLY | 55 | 47.220 | 41.492 | 47.077 | 1.00 | 38.78 | DIC |
| ATOM | 405 | N | GLY | 56 | 47.678 | 42.887 | 48.779 | 1.00 | 38.50 | DIC |
| ATOM | 406 | CA | GLY | 56 | 46.780 | 42.198 | 49.685 | 1.00 | 38.47 | DIC |
| ATOM | 407 | C | GLY | 56 | 47.417 | 40.973 | 50.309 | 1.00 | 38.75 | DIC |
| ATOM | 408 | O | GLY | 56 | 46.747 | 39.964 | 50.534 | 1.00 | 40.02 | DIC |
| ATOM | 409 | N | GLY | 57 | 48.716 | 41.061 | 50.586 | 1.00 | 37.85 | DIC |
| ATOM | 410 | CA | GLY | 57 | 49.424 | 39.948 | 51.193 | 1.00 | 35.96 | DIC |
| ATOM | 411 | C | GLY | 57 | 49.144 | 39.855 | 52.682 | 1.00 | 34.60 | DIC |
| ATOM | 412 | O | GLY | 57 | 49.441 | 38.845 | 53.321 | 1.00 | 34.23 | DIC |
| ATOM | 413 | N | ASN | 58 | 48.568 | 40.914 | 53.241 | 1.00 | 33.21 | DIC |
| ATOM | 414 | CA | ASN | 58 | 48.250 | 40.933 | 54.664 | 1.00 | 31.01 | DIC |
| ATOM | 415 | CB | ASN | 58 | 48.877 | 42.154 | 55.328 | 1.00 | 33.87 | DIC |
| ATOM | 416 | CG | ASN | 58 | 50.322 | 41.922 | 55.706 | 1.00 | 37.81 | DIC |
| ATOM | 417 | OD1 | ASN | 58 | 50.618 | 41.127 | 56.604 | 1.00 | 38.68 | DIC |
| ATOM | 418 | ND2 | ASN | 58 | 51.234 | 42.603 | 55.016 | 1.00 | 39.42 | DIC |
| ATOM | 419 | C | ASN | 58 | 46.763 | 40.889 | 54.965 | 1.00 | 28.44 | DIC |
| ATOM | 420 | O | ASN | 58 | 46.279 | 41.588 | 55.861 | 1.00 | 25.75 | DIC |
| ATOM | 421 | N | ILE | 59 | 46.035 | 40.070 | 54.211 | 1.00 | 24.88 | DIC |
| ATOM | 422 | CA | ILE | 59 | 44.607 | 39.927 | 54.445 | 1.00 | 23.41 | DIC |
| ATOM | 423 | CB | ILE | 59 | 43.763 | 40.859 | 53.526 | 1.00 | 23.71 | DIC |
| ATOM | 424 | CG2 | ILE | 59 | 43.989 | 40.514 | 52.055 | 1.00 | 22.32 | DIC |
| ATOM | 425 | CG1 | ILE | 59 | 42.282 | 40.740 | 53.909 | 1.00 | 21.89 | DIC |
| ATOM | 426 | CD | ILE | 59 | 41.398 | 41.852 | 53.361 | 1.00 | 22.80 | DIC |
| ATOM | 427 | C | ILE | 59 | 44.167 | 38.482 | 54.264 | 1.00 | 22.27 | DIC |
| ATOM | 428 | O | ILE | 59 | 44.400 | 37.863 | 53.221 | 1.00 | 21.93 | DIC |
| ATOM | 429 | N | ARG | 60 | 43.550 | 37.945 | 55.308 | 1.00 | 20.40 | DIC |
| ATOM | 430 | CA | ARG | 60 | 43.062 | 36.574 | 55.300 | 1.00 | 19.40 | DIC |
| ATOM | 431 | CB | ARG | 60 | 43.748 | 35.769 | 56.417 | 1.00 | 20.70 | DIC |
| ATOM | 432 | CG | ARG | 60 | 43.338 | 34.300 | 56.513 | 1.00 | 25.16 | DIC |
| ATOM | 433 | CD | ARG | 60 | 44.461 | 33.403 | 57.069 | 1.00 | 29.20 | DIC |
| ATOM | 434 | NE | ARG | 60 | 44.896 | 33.763 | 58.418 | 1.00 | 33.94 | DIC |
| ATOM | 435 | CZ | ARG | 60 | 45.736 | 34.757 | 58.707 | 1.00 | 36.79 | DIC |
| ATOM | 436 | NH1 | ARG | 60 | 46.250 | 35.507 | 57.740 | 1.00 | 38.29 | DIC |
| ATOM | 437 | NH2 | ARG | 60 | 46.068 | 35.000 | 59.971 | 1.00 | 36.32 | DIC |
| ATOM | 438 | C | ARG | 60 | 41.554 | 36.613 | 55.508 | 1.00 | 18.67 | DIC |
| ATOM | 439 | O | ARG | 60 | 41.062 | 37.256 | 56.441 | 1.00 | 19.14 | DIC |
| ATOM | 440 | N | PHE | 61 | 40.820 | 35.959 | 54.615 | 1.00 | 15.54 | DIC |
| ATOM | 441 | CA | PHE | 61 | 39.369 | 35.904 | 54.722 | 1.00 | 16.16 | DIC |
| ATOM | 442 | CB | PHE | 61 | 38.713 | 35.924 | 53.333 | 1.00 | 14.93 | DIC |
| ATOM | 443 | CG | PHE | 61 | 38.885 | 37.230 | 52.601 | 1.00 | 12.73 | DIC |
| ATOM | 444 | CD1 | PHE | 61 | 39.989 | 37.448 | 51.786 | 1.00 | 13.77 | DIC |
| ATOM | 445 | CD2 | PHE | 61 | 37.958 | 38.258 | 52.763 | 1.00 | 11.24 | DIC |
| ATOM | 446 | CE1 | PHE | 61 | 40.170 | 38.677 | 51.143 | 1.00 | 14.58 | DIC |
| ATOM | 447 | CE2 | PHE | 61 | 38.128 | 39.482 | 52.131 | 1.00 | 12.41 | DIC |
| ATOM | 448 | CZ | PHE | 61 | 39.236 | 39.696 | 51.319 | 1.00 | 11.76 | DIC |
| ATOM | 449 | C | PHE | 61 | 39.026 | 34.613 | 55.448 | 1.00 | 16.95 | DIC |
| ATOM | 450 | O | PHE | 61 | 39.542 | 33.554 | 55.105 | 1.00 | 18.27 | DIC |
| ATOM | 451 | N | ILE | 62 | 38.176 | 34.714 | 56.467 | 1.00 | 17.58 | DIC |
| ATOM | 452 | CA | ILE | 62 | 37.767 | 33.560 | 57.264 | 1.00 | 16.19 | DIC |
| ATOM | 453 | CB | ILE | 62 | 38.022 | 33.809 | 58.770 | 1.00 | 16.81 | DIC |
| ATOM | 454 | CG2 | ILE | 62 | 37.808 | 32.521 | 59.560 | 1.00 | 15.43 | DIC |
| ATOM | 455 | CG1 | ILE | 62 | 39.443 | 34.321 | 58.987 | 1.00 | 17.10 | DIC |
| ATOM | 456 | CD | ILE | 62 | 39.684 | 34.852 | 60.390 | 1.00 | 19.03 | DIC |
| ATOM | 457 | C | ILE | 62 | 36.271 | 33.328 | 57.075 | 1.00 | 16.68 | DIC |
| ATOM | 458 | O | ILE | 62 | 35.456 | 34.150 | 57.495 | 1.00 | 13.95 | DIC |
| ATOM | 459 | N | ASP | 63 | 35.911 | 32.207 | 56.457 | 1.00 | 17.73 | DIC |
| ATOM | 460 | CA | ASP | 63 | 34.507 | 31.906 | 56.219 | 1.00 | 20.50 | DIC |
| ATOM | 461 | CB | ASP | 63 | 34.354 | 30.648 | 55.352 | 1.00 | 24.57 | DIC |
| ATOM | 462 | CG | ASP | 63 | 34.848 | 30.853 | 53.926 | 1.00 | 28.34 | DIC |
| ATOM | 463 | OD1 | ASP | 63 | 34.508 | 31.890 | 53.317 | 1.00 | 31.73 | DIC |
| ATOM | 464 | OD2 | ASP | 63 | 35.566 | 29.971 | 53.412 | 1.00 | 32.70 | DIC |
| ATOM | 465 | C | ASP | 63 | 33.716 | 31.723 | 57.504 | 1.00 | 20.66 | DIC |
| ATOM | 466 | O | ASP | 63 | 34.147 | 31.036 | 58.436 | 1.00 | 20.12 | DIC |
| ATOM | 467 | N | VAL | 64 | 32.557 | 32.363 | 57.549 | 1.00 | 20.64 | DIC |
| ATOM | 468 | CA | VAL | 64 | 31.662 | 32.254 | 58.686 | 1.00 | 20.45 | DIC |
| ATOM | 469 | CB | VAL | 64 | 31.601 | 33.569 | 59.498 | 1.00 | 22.06 | DIC |
| ATOM | 470 | CG1 | VAL | 64 | 32.985 | 33.917 | 60.013 | 1.00 | 22.97 | DIC |
| ATOM | 471 | CG2 | VAL | 64 | 31.056 | 34.696 | 58.645 | 1.00 | 22.72 | DIC |
| ATOM | 472 | C | VAL | 64 | 30.290 | 31.924 | 58.116 | 1.00 | 20.57 | DIC |
| ATOM | 473 | O | VAL | 64 | 29.936 | 32.377 | 57.024 | 1.00 | 20.54 | DIC |
| ATOM | 474 | N | ASN | 65 | 29.536 | 31.107 | 58.840 | 1.00 | 19.87 | DIC |
| ATOM | 475 | CA | ASN | 65 | 28.203 | 30.709 | 58.406 | 1.00 | 20.85 | DIC |
| ATOM | 476 | CB | ASN | 65 | 27.898 | 29.282 | 58.869 | 1.00 | 21.23 | DIC |
| ATOM | 477 | CG | ASN | 65 | 26.615 | 28.732 | 58.264 | 1.00 | 24.15 | DIC |
| ATOM | 478 | OD1 | ASN | 65 | 25.670 | 29.478 | 57.988 | 1.00 | 24.03 | DIC |
| ATOM | 479 | ND2 | ASN | 65 | 26.572 | 27.418 | 58.067 | 1.00 | 22.28 | DIC |
| ATOM | 480 | C | ASN | 65 | 27.195 | 31.666 | 59.034 | 1.00 | 21.32 | DIC |
| ATOM | 481 | O | ASN | 65 | 26.916 | 31.586 | 60.229 | 1.00 | 20.43 | DIC |
| ATOM | 482 | N | PRO | 66 | 26.625 | 32.575 | 58.231 | 1.00 | 21.57 | DIC |
| ATOM | 483 | CD | PRO | 66 | 26.755 | 32.695 | 56.768 | 1.00 | 22.50 | DIC |
| ATOM | 484 | CA | PRO | 66 | 25.649 | 33.535 | 58.754 | 1.00 | 22.52 | DIC |
| ATOM | 485 | CB | PRO | 66 | 25.280 | 34.359 | 57.519 | 1.00 | 23.12 | DIC |
| ATOM | 486 | CG | PRO | 66 | 25.471 | 33.391 | 56.392 | 1.00 | 22.95 | DIC |
| ATOM | 487 | C | PRO | 66 | 24.440 | 32.880 | 59.421 | 1.00 | 21.35 | DIC |
| ATOM | 488 | O | PRO | 66 | 23.817 | 33.467 | 60.306 | 1.00 | 20.47 | DIC |
| ATOM | 489 | N | ALA | 67 | 24.124 | 31.657 | 59.009 | 1.00 | 20.26 | DIC |
| ATOM | 490 | CA | ALA | 67 | 22.992 | 30.937 | 59.577 | 1.00 | 20.00 | DIC |
| ATOM | 491 | CB | ALA | 67 | 22.771 | 29.619 | 58.823 | 1.00 | 21.70 | DIC |
| ATOM | 492 | C | ALA | 67 | 23.180 | 30.662 | 61.069 | 1.00 | 20.28 | DIC |
| ATOM | 493 | O | ALA | 67 | 22.202 | 30.487 | 61.797 | 1.00 | 19.39 | DIC |
| ATOM | 494 | N | ASP | 68 | 24.430 | 30.624 | 61.527 | 1.00 | 18.25 | DIC |
| ATOM | 495 | CA | ASP | 68 | 24.702 | 30.366 | 62.937 | 1.00 | 17.98 | DIC |
| ATOM | 496 | CB | ASP | 68 | 26.196 | 30.512 | 63.249 | 1.00 | 18.00 | DIC |
| ATOM | 497 | CG | ASP | 68 | 27.037 | 29.385 | 62.680 | 1.00 | 19.22 | DIC |
| ATOM | 498 | OD1 | ASP | 68 | 28.264 | 29.391 | 62.924 | 1.00 | 20.73 | DIC |
| ATOM | 499 | OD2 | ASP | 68 | 26.488 | 28.500 | 61.994 | 1.00 | 21.66 | DIC |
| ATOM | 500 | C | ASP | 68 | 23.941 | 31.323 | 63.853 | 1.00 | 17.42 | DIC |
| ATOM | 501 | O | ASP | 68 | 23.596 | 30.971 | 64.981 | 1.00 | 16.39 | DIC |
| ATOM | 502 | N | PHE | 69 | 23.679 | 32.531 | 63.371 | 1.00 | 16.56 | DIC |
| ATOM | 503 | CA | PHE | 69 | 23.005 | 33.527 | 64.192 | 1.00 | 18.20 | DIC |
| ATOM | 504 | CB | PHE | 69 | 23.823 | 34.821 | 64.160 | 1.00 | 18.04 | DIC |
| ATOM | 505 | CG | PHE | 69 | 25.314 | 34.583 | 64.118 | 1.00 | 18.37 | DIC |
| ATOM | 506 | CD1 | PHE | 69 | 25.983 | 34.473 | 62.898 | 1.00 | 18.98 | DIC |
| ATOM | 507 | CD2 | PHE | 69 | 26.039 | 34.431 | 65.293 | 1.00 | 16.47 | DIC |
| ATOM | 508 | CE1 | PHE | 69 | 27.354 | 34.215 | 62.852 | 1.00 | 18.40 | DIC |
| ATOM | 509 | CE2 | PHE | 69 | 27.410 | 34.172 | 65.264 | 1.00 | 16.90 | DIC |
| ATOM | 510 | CZ | PHE | 69 | 28.071 | 34.063 | 64.039 | 1.00 | 18.55 | DIC |
| ATOM | 511 | C | PHE | 69 | 21.565 | 33.796 | 63.778 | 1.00 | 20.05 | DIC |
| ATOM | 512 | O | PHE | 69 | 20.965 | 34.796 | 64.181 | 1.00 | 19.59 | DIC |
| ATOM | 513 | N | ALA | 70 | 21.004 | 32.884 | 62.995 | 1.00 | 21.70 | DIC |
| ATOM | 514 | CA | ALA | 70 | 19.638 | 33.030 | 62.509 | 1.00 | 23.13 | DIC |
| ATOM | 515 | CB | ALA | 70 | 19.253 | 31.812 | 61.678 | 1.00 | 24.32 | DIC |
| ATOM | 516 | C | ALA | 70 | 18.597 | 33.257 | 63.603 | 1.00 | 24.21 | DIC |
| ATOM | 517 | O | ALA | 70 | 17.553 | 33.857 | 63.348 | 1.00 | 25.81 | DIC |
| ATOM | 518 | N | GLY | 71 | 18.867 | 32.782 | 64.814 | 1.00 | 23.19 | DIC |
| ATOM | 519 | CA | GLY | 71 | 17.901 | 32.964 | 65.883 | 1.00 | 23.35 | DIC |
| ATOM | 520 | C | GLY | 71 | 17.929 | 34.315 | 66.583 | 1.00 | 22.92 | DIC |
| ATOM | 521 | O | GLY | 71 | 17.009 | 34.642 | 67.338 | 1.00 | 21.65 | DIC |
| ATOM | 522 | N | PHE | 72 | 18.966 | 35.109 | 66.331 | 1.00 | 21.05 | DIC |
| ATOM | 523 | CA | PHE | 72 | 19.105 | 36.415 | 66.976 | 1.00 | 20.69 | DIC |
| ATOM | 524 | CB | PHE | 72 | 20.592 | 36.786 | 67.069 | 1.00 | 20.82 | DIC |
| ATOM | 525 | CG | PHE | 72 | 21.414 | 35.823 | 67.901 | 1.00 | 22.40 | DIC |
| ATOM | 526 | CD1 | PHE | 72 | 22.787 | 36.003 | 68.041 | 1.00 | 21.53 | DIC |
| ATOM | 527 | CD2 | PHE | 72 | 20.817 | 34.737 | 68.540 | 1.00 | 22.26 | DIC |
| ATOM | 528 | CE1 | PHE | 72 | 23.554 | 35.116 | 68.803 | 1.00 | 23.86 | DIC |
| ATOM | 529 | CE2 | PHE | 72 | 21.573 | 33.843 | 69.305 | 1.00 | 23.97 | DIC |
| ATOM | 530 | CZ | PHE | 72 | 22.946 | 34.033 | 69.437 | 1.00 | 23.46 | DIC |
| ATOM | 531 | C | PHE | 72 | 18.316 | 37.539 | 66.296 | 1.00 | 20.09 | DIC |
| ATOM | 532 | O | PHE | 72 | 18.196 | 37.579 | 65.074 | 1.00 | 21.80 | DIC |
| ATOM | 533 | N | PRO | 73 | 17.783 | 38.480 | 67.090 | 1.00 | 19.37 | DIC |
| ATOM | 534 | CD | PRO | 73 | 18.031 | 38.670 | 68.529 | 1.00 | 19.40 | DIC |
| ATOM | 535 | CA | PRO | 73 | 17.002 | 39.599 | 66.558 | 1.00 | 18.84 | DIC |
| ATOM | 536 | CB | PRO | 73 | 16.675 | 40.418 | 67.807 | 1.00 | 19.67 | DIC |
| ATOM | 537 | CG | PRO | 73 | 17.826 | 40.159 | 68.687 | 1.00 | 19.66 | DIC |
| ATOM | 538 | C | PRO | 73 | 17.711 | 40.418 | 65.495 | 1.00 | 18.90 | DIC |
| ATOM | 539 | O | PRO | 73 | 18.905 | 40.705 | 65.596 | 1.00 | 16.76 | DIC |
| ATOM | 540 | N | LEU | 74 | 16.956 | 40.790 | 64.470 | 1.00 | 18.84 | DIC |
| ATOM | 541 | CA | LEU | 74 | 17.493 | 41.583 | 63.378 | 1.00 | 21.06 | DIC |
| ATOM | 542 | CB | LEU | 74 | 17.769 | 40.673 | 62.176 | 1.00 | 22.16 | DIC |
| ATOM | 543 | CG | LEU | 74 | 18.916 | 41.043 | 61.231 | 1.00 | 22.74 | DIC |
| ATOM | 544 | CD1 | LEU | 74 | 20.240 | 41.080 | 61.989 | 1.00 | 23.10 | DIC |
| ATOM | 545 | CD2 | LEU | 74 | 18.983 | 40.026 | 60.114 | 1.00 | 23.73 | DIC |
| ATOM | 546 | C | LEU | 74 | 16.438 | 42.637 | 63.040 | 1.00 | 21.30 | DIC |
| ATOM | 547 | O | LEU | 74 | 15.758 | 42.540 | 62.021 | 1.00 | 22.75 | DIC |
| ATOM | 548 | N | ASN | 75 | 16.308 | 43.634 | 63.915 | 1.00 | 21.53 | DIC |
| ATOM | 549 | CA | ASN | 75 | 15.329 | 44.708 | 63.749 | 1.00 | 20.41 | DIC |
| ATOM | 550 | CB | ASN | 75 | 14.716 | 45.101 | 65.125 | 1.00 | 22.53 | DIC |
| ATOM | 551 | CG | ASN | 75 | 15.767 | 45.292 | 66.259 | 1.00 | 25.80 | DIC |
| ATOM | 552 | OD1 | ASN | 75 | 16.458 | 44.349 | 66.665 | 1.00 | 23.29 | DIC |
| ATOM | 553 | ND2 | ASN | 75 | 15.854 | 46.520 | 66.791 | 1.00 | 26.49 | DIC |
| ATOM | 554 | C | ASN | 75 | 15.822 | 45.968 | 63.012 | 1.00 | 20.20 | DIC |
| ATOM | 555 | O | ASN | 75 | 15.036 | 46.654 | 62.355 | 1.00 | 18.24 | DIC |
| ATOM | 556 | N | ILE | 76 | 17.116 | 46.265 | 63.111 | 1.00 | 16.30 | DIC |
| ATOM | 557 | CA | ILE | 76 | 17.676 | 47.450 | 62.467 | 1.00 | 14.30 | DIC |
| ATOM | 558 | CB | ILE | 76 | 18.998 | 47.836 | 63.155 | 1.00 | 15.53 | DIC |
| ATOM | 559 | CG2 | ILE | 76 | 19.496 | 49.183 | 62.643 | 1.00 | 14.83 | DIC |
| ATOM | 560 | CG1 | ILE | 76 | 18.753 | 47.925 | 64.668 | 1.00 | 15.63 | DIC |
| ATOM | 561 | CD | ILE | 76 | 20.000 | 48.191 | 65.496 | 1.00 | 16.08 | DIC |
| ATOM | 562 | C | ILE | 76 | 17.869 | 47.172 | 60.976 | 1.00 | 13.06 | DIC |
| ATOM | 563 | O | ILE | 76 | 18.732 | 46.398 | 60.578 | 1.00 | 11.46 | DIC |
| ATOM | 564 | N | ARG | 77 | 17.036 | 47.813 | 60.161 | 1.00 | 12.41 | DIC |
| ATOM | 565 | CA | ARG | 77 | 17.033 | 47.606 | 58.719 | 1.00 | 11.27 | DIC |
| ATOM | 566 | CB | ARG | 77 | 16.016 | 48.544 | 58.071 | 1.00 | 11.91 | DIC |
| ATOM | 567 | CG | ARG | 77 | 15.774 | 48.285 | 56.589 | 1.00 | 14.32 | DIC |
| ATOM | 568 | CD | ARG | 77 | 14.766 | 49.291 | 56.023 | 1.00 | 17.97 | DIC |
| ATOM | 569 | NE | ARG | 77 | 14.418 | 48.999 | 54.637 | 1.00 | 19.40 | DIC |
| ATOM | 570 | CZ | ARG | 77 | 14.017 | 49.909 | 53.755 | 1.00 | 20.80 | DIC |
| ATOM | 571 | NH1 | ARG | 77 | 13.910 | 51.182 | 54.108 | 1.00 | 20.49 | DIC |
| ATOM | 572 | NH2 | ARG | 77 | 13.730 | 49.543 | 52.512 | 1.00 | 20.14 | DIC |
| ATOM | 573 | C | ARG | 77 | 18.363 | 47.718 | 57.980 | 1.00 | 10.68 | DIC |
| ATOM | 574 | O | ARG | 77 | 18.642 | 46.905 | 57.102 | 1.00 | 9.93 | DIC |
| ATOM | 575 | N | HIS | 78 | 19.184 | 48.709 | 58.318 | 1.00 | 9.86 | DIC |
| ATOM | 576 | CA | HIS | 78 | 20.455 | 48.876 | 57.613 | 1.00 | 11.66 | DIC |
| ATOM | 577 | CB | HIS | 78 | 20.975 | 50.316 | 57.782 | 1.00 | 12.15 | DIC |
| ATOM | 578 | CG | HIS | 78 | 21.469 | 50.635 | 59.160 | 1.00 | 11.23 | DIC |
| ATOM | 579 | CD2 | HIS | 78 | 20.830 | 51.149 | 60.238 | 1.00 | 9.90 | DIC |
| ATOM | 580 | ND1 | HIS | 78 | 22.779 | 50.447 | 59.545 | 1.00 | 14.19 | DIC |
| ATOM | 581 | CE1 | HIS | 78 | 22.927 | 50.834 | 60.801 | 1.00 | 11.70 | DIC |
| ATOM | 582 | NE2 | HIS | 78 | 21.758 | 51.264 | 61.244 | 1.00 | 13.16 | DIC |
| ATOM | 583 | C | HIS | 78 | 21.537 | 47.879 | 58.022 | 1.00 | 11.09 | DIC |
| ATOM | 584 | O | HIS | 78 | 22.637 | 47.898 | 57.474 | 1.00 | 10.55 | DIC |
| ATOM | 585 | N | ILE | 79 | 21.219 | 46.989 | 58.961 | 1.00 | 9.64 | DIC |
| ATOM | 586 | CA | ILE | 79 | 22.199 | 46.016 | 59.438 | 1.00 | 8.79 | DIC |
| ATOM | 587 | CB | ILE | 79 | 22.327 | 46.110 | 60.980 | 1.00 | 8.64 | DIC |
| ATOM | 588 | CG2 | ILE | 79 | 23.316 | 45.068 | 61.493 | 1.00 | 7.07 | DIC |
| ATOM | 589 | CG1 | ILE | 79 | 22.762 | 47.529 | 61.374 | 1.00 | 8.17 | DIC |
| ATOM | 590 | CD | ILE | 79 | 22.723 | 47.807 | 62.875 | 1.00 | 9.48 | DIC |
| ATOM | 591 | C | ILE | 79 | 21.874 | 44.568 | 59.053 | 1.00 | 10.57 | DIC |
| ATOM | 592 | O | ILE | 79 | 20.769 | 44.088 | 59.298 | 1.00 | 10.81 | DIC |
| ATOM | 593 | N | SER | 80 | 22.838 | 43.871 | 58.453 | 1.00 | 11.32 | DIC |
| ATOM | 594 | CA | SER | 80 | 22.632 | 42.473 | 58.065 | 1.00 | 10.85 | DIC |
| ATOM | 595 | CB | SER | 80 | 23.416 | 42.135 | 56.800 | 1.00 | 11.37 | DIC |
| ATOM | 596 | OG | SER | 80 | 24.804 | 42.239 | 57.045 | 1.00 | 11.89 | DIC |
| ATOM | 597 | C | SER | 80 | 23.098 | 41.556 | 59.193 | 1.00 | 11.08 | DIC |
| ATOM | 598 | O | SER | 80 | 23.788 | 41.999 | 60.117 | 1.00 | 11.39 | DIC |
| ATOM | 599 | N | ILE | 81 | 22.722 | 40.281 | 59.094 | 1.00 | 10.23 | DIC |
| ATOM | 600 | CA | ILE | 81 | 23.038 | 39.251 | 60.087 | 1.00 | 12.43 | DIC |
| ATOM | 601 | CB | ILE | 81 | 22.449 | 37.868 | 59.640 | 1.00 | 13.92 | DIC |
| ATOM | 602 | CG2 | ILE | 81 | 23.114 | 37.404 | 58.354 | 1.00 | 14.49 | DIC |
| ATOM | 603 | CG1 | ILE | 81 | 22.637 | 36.816 | 60.738 | 1.00 | 16.79 | DIC |
| ATOM | 604 | CD | ILE | 81 | 21.858 | 37.101 | 62.008 | 1.00 | 21.00 | DIC |
| ATOM | 605 | C | ILE | 81 | 24.533 | 39.113 | 60.388 | 1.00 | 11.38 | DIC |
| ATOM | 606 | O | ILE | 81 | 24.921 | 38.716 | 61.483 | 1.00 | 11.02 | DIC |
| ATOM | 607 | N | THR | 82 | 25.368 | 39.460 | 59.421 | 1.00 | 11.66 | DIC |
| ATOM | 608 | CA | THR | 82 | 26.816 | 39.382 | 59.590 | 1.00 | 13.55 | DIC |
| ATOM | 609 | CB | THR | 82 | 27.525 | 39.881 | 58.309 | 1.00 | 12.95 | DIC |
| ATOM | 610 | OG1 | THR | 82 | 27.238 | 38.981 | 57.235 | 1.00 | 15.78 | DIC |
| ATOM | 611 | OG2 | THR | 82 | 29.027 | 39.953 | 58.505 | 1.00 | 13.95 | DIC |
| ATOM | 612 | C | THR | 82 | 27.305 | 40.193 | 60.801 | 1.00 | 13.04 | DIC |
| ATOM | 613 | O | THR | 82 | 28.423 | 40.001 | 61.283 | 1.00 | 14.43 | DIC |
| ATOM | 614 | N | THR | 83 | 26.468 | 41.100 | 61.293 | 1.00 | 12.94 | DIC |
| ATOM | 615 | CA | THR | 83 | 26.843 | 41.925 | 62.444 | 1.00 | 10.16 | DIC |
| ATOM | 616 | CB | THR | 83 | 25.762 | 42.993 | 62.736 | 1.00 | 8.84 | DIC |
| ATOM | 617 | OG1 | THR | 83 | 26.255 | 43.900 | 63.729 | 1.00 | 8.21 | DIC |
| ATOM | 618 | CG2 | THR | 83 | 24.473 | 42.345 | 63.242 | 1.00 | 7.30 | DIC |
| ATOM | 619 | C | THR | 83 | 27.079 | 41.088 | 63.712 | 1.00 | 9.87 | DIC |
| ATOM | 620 | O | THR | 83 | 27.744 | 41.536 | 64.654 | 1.00 | 8.73 | DIC |
| ATOM | 621 | N | TYR | 84 | 26.545 | 39.869 | 63.732 | 1.00 | 8.10 | DIC |
| ATOM | 622 | CA | TYR | 84 | 26.719 | 38.990 | 64.889 | 1.00 | 9.50 | DIC |
| ATOM | 623 | CB | TYR | 84 | 25.508 | 38.073 | 65.061 | 1.00 | 8.94 | DIC |
| ATOM | 624 | CG | TYR | 84 | 24.278 | 38.731 | 65.627 | 1.00 | 10.26 | DIC |
| ATOM | 625 | CD1 | TYR | 84 | 23.194 | 39.044 | 64.805 | 1.00 | 10.22 | DIC |
| ATOM | 626 | CE1 | TYR | 84 | 22.036 | 39.613 | 65.327 | 1.00 | 10.08 | DIC |
| ATOM | 627 | CD2 | TYR | 84 | 24.179 | 39.011 | 66.994 | 1.00 | 10.47 | DIC |
| ATOM | 628 | CE2 | TYR | 84 | 23.028 | 39.581 | 67.527 | 1.00 | 9.14 | DIC |
| ATOM | 629 | CZ | TYR | 84 | 21.959 | 39.875 | 66.685 | 1.00 | 10.95 | DIC |
| ATOM | 630 | OH | TYR | 84 | 20.806 | 40.406 | 67.205 | 1.00 | 13.06 | DIC |
| ATOM | 631 | C | TYR | 84 | 27.965 | 38.104 | 64.835 | 1.00 | 9.18 | DIC |
| ATOM | 632 | O | TYR | 84 | 28.314 | 37.471 | 65.832 | 1.00 | 10.15 | DIC |
| ATOM | 633 | N | ALA | 85 | 28.641 | 38.054 | 63.689 | 1.00 | 9.06 | DIC |
| ATOM | 634 | CA | ALA | 85 | 29.814 | 37.185 | 63.554 | 1.00 | 8.72 | DIC |
| ATOM | 635 | CB | ALA | 85 | 30.394 | 37.294 | 62.145 | 1.00 | 9.77 | DIC |
| ATOM | 636 | C | ALA | 85 | 30.912 | 37.423 | 64.581 | 1.00 | 9.19 | DIC |
| ATOM | 637 | O | ALA | 85 | 31.599 | 36.486 | 64.993 | 1.00 | 8.82 | DIC |
| ATOM | 638 | N | ARG | 86 | 31.074 | 38.669 | 65.003 | 1.00 | 9.47 | DIC |
| ATOM | 639 | CA | ARG | 86 | 32.111 | 39.003 | 65.966 | 1.00 | 9.96 | DIC |
| ATOM | 640 | CB | ARG | 86 | 32.051 | 40.502 | 66.294 | 1.00 | 9.71 | DIC |
| ATOM | 641 | CG | ARG | 86 | 30.811 | 40.949 | 67.048 | 1.00 | 9.90 | DIC |
| ATOM | 642 | CD | ARG | 86 | 30.656 | 42.466 | 67.001 | 1.00 | 8.17 | DIC |
| ATOM | 643 | NE | ARG | 86 | 29.919 | 42.902 | 65.815 | 1.00 | 7.34 | DIC |
| ATOM | 644 | CZ | ARG | 86 | 29.791 | 44.169 | 65.430 | 1.00 | 8.06 | DIC |
| ATOM | 645 | NH1 | ARG | 86 | 30.357 | 45.142 | 66.133 | 1.00 | 6.00 | DIC |
| ATOM | 646 | NH2 | ARG | 86 | 29.079 | 44.464 | 64.347 | 1.00 | 8.41 | DIC |
| ATOM | 647 | C | ARG | 86 | 32.016 | 38.165 | 67.243 | 1.00 | 9.15 | DIC |
| ATOM | 648 | O | ARG | 86 | 33.030 | 37.911 | 67.896 | 1.00 | 9.85 | DIC |
| ATOM | 649 | N | LEU | 87 | 30.809 | 37.724 | 67.593 | 1.00 | 10.06 | DIC |
| ATOM | 650 | CA | LEU | 87 | 30.607 | 36.909 | 68.799 | 1.00 | 10.59 | DIC |
| ATOM | 651 | CB | LEU | 87 | 29.124 | 36.563 | 68.957 | 1.00 | 11.67 | DIC |
| ATOM | 652 | CG | LEU | 87 | 28.147 | 37.736 | 69.102 | 1.00 | 12.74 | DIC |
| ATOM | 653 | CD1 | LEU | 87 | 26.726 | 37.198 | 69.137 | 1.00 | 13.49 | DIC |
| ATOM | 654 | CD2 | LEU | 87 | 28.456 | 38.527 | 70.368 | 1.00 | 11.69 | DIC |
| ATOM | 655 | C | LEU | 87 | 31.435 | 35.613 | 68.832 | 1.00 | 11.32 | DIC |
| ATOM | 656 | O | LEU | 87 | 31.747 | 35.096 | 69.907 | 1.00 | 9.34 | DIC |
| ATOM | 657 | N | LYS | 88 | 31.781 | 35.085 | 67.658 | 1.00 | 12.29 | DIC |
| ATOM | 658 | CA | LYS | 88 | 32.575 | 33.849 | 67.575 | 1.00 | 12.77 | DIC |
| ATOM | 659 | CB | LYS | 88 | 31.995 | 32.914 | 66.506 | 1.00 | 13.12 | DIC |
| ATOM | 660 | CG | LYS | 88 | 30.724 | 32.181 | 66.926 | 1.00 | 14.09 | DIC |
| ATOM | 661 | CD | LYS | 88 | 30.390 | 31.098 | 65.907 | 1.00 | 14.18 | DIC |
| ATOM | 662 | CE | LYS | 88 | 29.287 | 30.182 | 66.387 | 1.00 | 16.96 | DIC |
| ATOM | 663 | NZ | LYS | 88 | 29.049 | 29.101 | 65.386 | 1.00 | 15.57 | DIC |
| ATOM | 664 | C | LYS | 88 | 34.043 | 34.120 | 67.252 | 1.00 | 12.47 | DIC |
| ATOM | 665 | O | LYS | 88 | 34.755 | 33.247 | 66.754 | 1.00 | 13.10 | DIC |
| ATOM | 666 | N | LEU | 89 | 34.495 | 35.332 | 67.540 | 1.00 | 11.63 | DIC |
| ATOM | 667 | CA | LEU | 89 | 35.874 | 35.710 | 67.263 | 1.00 | 13.26 | DIC |
| ATOM | 668 | CB | LEU | 89 | 36.117 | 37.141 | 67.760 | 1.00 | 14.71 | DIC |
| ATOM | 669 | CG | LEU | 89 | 36.901 | 38.093 | 66.853 | 1.00 | 18.28 | DIC |
| ATOM | 670 | CD1 | LEU | 89 | 36.517 | 37.918 | 65.401 | 1.00 | 18.01 | DIC |
| ATOM | 671 | CD2 | LEU | 89 | 36.632 | 39.520 | 67.302 | 1.00 | 19.23 | DIC |
| ATOM | 672 | C | LEU | 89 | 36.854 | 34.725 | 67.906 | 1.00 | 13.22 | DIC |
| ATOM | 673 | O | LEU | 89 | 37.878 | 34.378 | 67.314 | 1.00 | 14.28 | DIC |
| ATOM | 674 | N | GLY | 90 | 36.528 | 34.262 | 69.109 | 1.00 | 12.35 | DIC |
| ATOM | 675 | CA | GLY | 90 | 37.381 | 33.310 | 69.797 | 1.00 | 12.11 | DIC |
| ATOM | 676 | C | GLY | 90 | 37.553 | 32.001 | 69.041 | 1.00 | 14.28 | DIC |
| ATOM | 677 | O | GLY | 90 | 38.561 | 31.310 | 69.213 | 1.00 | 12.59 | DIC |
| ATOM | 678 | N | GLU | 91 | 36.577 | 31.667 | 68.197 | 1.00 | 14.29 | DIC |
| ATOM | 679 | CA | GLU | 91 | 36.612 | 30.440 | 67.395 | 1.00 | 15.06 | DIC |
| ATOM | 680 | CB | GLU | 91 | 35.194 | 29.912 | 67.159 | 1.00 | 16.79 | DIC |
| ATOM | 681 | CG | GLU | 91 | 34.447 | 29.384 | 68.383 | 1.00 | 19.76 | DIC |
| ATOM | 682 | CD | GLU | 91 | 33.104 | 28.774 | 67.992 | 1.00 | 23.18 | DIC |
| ATOM | 683 | OE1 | GLU | 91 | 33.086 | 27.933 | 67.064 | 1.00 | 22.19 | DIC |
| ATOM | 684 | OE2 | GLU | 91 | 32.071 | 29.132 | 68.606 | 1.00 | 23.20 | DIC |
| ATOM | 685 | C | GLU | 91 | 37.259 | 30.635 | 66.016 | 1.00 | 14.78 | DIC |
| ATOM | 686 | O | GLU | 91 | 37.855 | 29.705 | 65.465 | 1.00 | 14.56 | DIC |
| ATOM | 687 | N | TYR | 92 | 37.119 | 31.839 | 65.467 | 1.00 | 14.84 | DIC |
| ATOM | 688 | CA | TYR | 92 | 37.636 | 32.175 | 64.140 | 1.00 | 16.06 | DIC |
| ATOM | 689 | CB | TYR | 92 | 36.850 | 33.354 | 63.549 | 1.00 | 14.49 | DIC |
| ATOM | 690 | CG | TYR | 92 | 35.356 | 33.128 | 63.399 | 1.00 | 15.73 | DIC |
| ATOM | 691 | CD1 | TYR | 92 | 34.856 | 31.930 | 62.889 | 1.00 | 15.13 | DIC |
| ATOM | 692 | CE1 | TYR | 92 | 33.483 | 31.741 | 62.698 | 1.00 | 16.39 | DIC |
| ATOM | 693 | CD2 | TYR | 92 | 34.445 | 34.139 | 63.720 | 1.00 | 15.08 | DIC |
| ATOM | 694 | CE2 | TYR | 92 | 33.073 | 33.962 | 63.532 | 1.00 | 16.55 | DIC |
| ATOM | 695 | CZ | TYR | 92 | 32.601 | 32.762 | 63.019 | 1.00 | 15.33 | DIC |
| ATOM | 696 | OH | TYR | 92 | 31.254 | 32.591 | 62.813 | 1.00 | 15.89 | DIC |
| ATOM | 697 | C | TYR | 92 | 39.122 | 32.516 | 64.050 | 1.00 | 16.64 | DIC |
| ATOM | 698 | O | TYR | 92 | 39.749 | 32.286 | 63.025 | 1.00 | 18.19 | DIC |
| ATOM | 699 | N | ILE | 93 | 39.680 | 33.084 | 65.109 | 1.00 | 18.45 | DIC |
| ATOM | 700 | CA | ILE | 93 | 41.085 | 33.468 | 65.089 | 1.00 | 19.66 | DIC |
| ATOM | 701 | CB | ILE | 93 | 41.221 | 35.001 | 65.239 | 1.00 | 20.11 | DIC |
| ATOM | 702 | CG2 | ILE | 93 | 42.690 | 35.389 | 65.380 | 1.00 | 18.82 | DIC |
| ATOM | 703 | CG1 | ILE | 93 | 40.562 | 35.684 | 64.031 | 1.00 | 19.68 | DIC |
| ATOM | 704 | CD | ILE | 93 | 40.589 | 37.195 | 64.072 | 1.00 | 20.89 | DIC |
| ATOM | 705 | C | ILE | 93 | 41.880 | 32.761 | 66.175 | 1.00 | 20.25 | DIC |
| ATOM | 706 | O | ILE | 93 | 41.553 | 32.845 | 67.361 | 1.00 | 19.56 | DIC |
| ATOM | 707 | N | ALA | 94 | 42.934 | 32.070 | 65.763 | 1.00 | 20.40 | DIC |
| ATOM | 708 | CA | ALA | 94 | 43.753 | 31.323 | 66.705 | 1.00 | 23.20 | DIC |
| ATOM | 709 | CB | ALA | 94 | 43.888 | 29.890 | 66.226 | 1.00 | 23.20 | DIC |
| ATOM | 710 | C | ALA | 94 | 45.136 | 31.923 | 66.921 | 1.00 | 24.59 | DIC |
| ATOM | 711 | O | ALA | 94 | 45.707 | 31.804 | 68.009 | 1.00 | 24.30 | DIC |
| ATOM | 712 | N | ASP | 95 | 45.660 | 32.574 | 65.888 | 1.00 | 26.69 | DIC |
| ATOM | 713 | CA | ASP | 95 | 46.995 | 33.163 | 65.930 | 1.00 | 29.50 | DIC |
| ATOM | 714 | CB | ASP | 95 | 47.572 | 33.219 | 64.509 | 1.00 | 31.58 | DIC |
| ATOM | 715 | CG | ASP | 95 | 46.640 | 33.908 | 63.524 | 1.00 | 35.84 | DIC |
| ATOM | 716 | OD1 | ASP | 95 | 47.039 | 34.104 | 62.355 | 1.00 | 38.60 | DIC |
| ATOM | 717 | OD2 | ASP | 95 | 45.504 | 34.252 | 63.913 | 1.00 | 38.61 | DIC |
| ATOM | 718 | C | ASP | 95 | 47.147 | 34.542 | 66.580 | 1.00 | 29.37 | DIC |
| ATOM | 719 | O | ASP | 95 | 48.141 | 35.224 | 66.333 | 1.00 | 31.82 | DIC |
| ATOM | 720 | N | GYS | 96 | 46.193 | 34.958 | 67.408 | 1.00 | 26.90 | DIC |
| ATOM | 721 | CA | GYS | 96 | 46.298 | 36.268 | 68.052 | 1.00 | 25.49 | DIC |
| ATOM | 722 | CB | GYS | 96 | 45.516 | 37.325 | 67.260 | 1.00 | 25.52 | DIC |
| ATOM | 723 | SG | GYS | 96 | 46.072 | 37.567 | 65.572 | 1.00 | 24.86 | DIC |
| ATOM | 724 | C | GYS | 96 | 45.793 | 36.288 | 69.484 | 1.00 | 24.23 | DIC |
| ATOM | 725 | O | GYS | 96 | 44.733 | 35.738 | 69.785 | 1.00 | 25.00 | DIC |
| ATOM | 726 | N | ASP | 97 | 46.549 | 36.927 | 70.368 | 1.00 | 22.41 | DIC |
| ATOM | 727 | CA | ASP | 97 | 46.122 | 37.044 | 71.756 | 1.00 | 22.45 | DIC |
| ATOM | 728 | CB | ASP | 97 | 47.326 | 37.111 | 72.689 | 1.00 | 22.93 | DIC |
| ATOM | 729 | CG | ASP | 97 | 47.968 | 35.760 | 72.901 | 1.00 | 24.22 | DIC |
| ATOM | 730 | OD1 | ASP | 97 | 49.053 | 35.713 | 73.516 | 1.00 | 26.27 | DIC |
| ATOM | 731 | OD2 | ASP | 97 | 47.384 | 34.748 | 72.457 | 1.00 | 25.38 | DIC |
| ATOM | 732 | C | ASP | 97 | 45.297 | 38.317 | 71.891 | 1.00 | 21.90 | DIC |
| ATOM | 733 | O | ASP | 97 | 44.517 | 38.471 | 72.830 | 1.00 | 23.11 | DIC |
| ATOM | 734 | N | LYS | 98 | 45.473 | 39.226 | 70.939 | 1.00 | 19.23 | DIC |
| ATOM | 735 | CA | LYS | 98 | 44.749 | 40.494 | 70.948 | 1.00 | 18.34 | DIC |
| ATOM | 736 | CB | LYS | 98 | 45.573 | 41.576 | 71.657 | 1.00 | 20.35 | DIC |
| ATOM | 737 | CG | LYS | 98 | 44.979 | 42.983 | 71.560 | 1.00 | 22.09 | DIC |
| ATOM | 738 | CD | LYS | 98 | 45.971 | 44.075 | 71.993 | 1.00 | 22.26 | DIC |
| ATOM | 739 | CE | LYS | 98 | 46.314 | 43.997 | 73.472 | 1.00 | 22.69 | DIC |
| ATOM | 740 | NZ | LYS | 98 | 46.981 | 45.238 | 73.980 | 1.00 | 20.82 | DIC |
| ATOM | 741 | C | LYS | 98 | 44.471 | 40.933 | 69.519 | 1.00 | 16.18 | DIC |
| ATOM | 742 | O | LYS | 98 | 45.324 | 40.795 | 68.642 | 1.00 | 13.58 | DIC |
| ATOM | 743 | N | VAL | 99 | 43.264 | 41.434 | 69.284 | 1.00 | 14.00 | DIC |
| ATOM | 744 | CA | VAL | 99 | 42.891 | 41.917 | 67.963 | 1.00 | 13.34 | DIC |
| ATOM | 745 | CB | VAL | 99 | 42.064 | 40.881 | 67.170 | 1.00 | 12.63 | DIC |
| ATOM | 746 | CG1 | VAL | 99 | 42.891 | 39.626 | 66.923 | 1.00 | 11.07 | DIC |
| ATOM | 747 | CG2 | VAL | 99 | 40.782 | 40.546 | 67.922 | 1.00 | 13.64 | DIC |
| ATOM | 748 | C | VAL | 99 | 42.058 | 43.169 | 68.141 | 1.00 | 13.18 | DIC |
| ATOM | 749 | O | VAL | 99 | 41.428 | 43.361 | 69.180 | 1.00 | 12.75 | DIC |
| ATOM | 750 | N | LEU | 100 | 42.081 | 44.033 | 67.136 | 1.00 | 11.89 | DIC |
| ATOM | 751 | CA | LEU | 100 | 41.304 | 45.257 | 67.181 | 1.00 | 12.48 | DIC |
| ATOM | 752 | CB | LEU | 100 | 42.183 | 46.473 | 66.870 | 1.00 | 13.07 | DIC |
| ATOM | 753 | CG | LEU | 100 | 41.498 | 47.849 | 66.824 | 1.00 | 16.10 | DIC |
| ATOM | 754 | CD1 | LEU | 100 | 40.525 | 47.996 | 67.984 | 1.00 | 15.54 | DIC |
| ATOM | 755 | CD2 | LEU | 100 | 42.558 | 48.940 | 66.881 | 1.00 | 15.51 | DIC |
| ATOM | 756 | C | LEU | 100 | 40.200 | 45.122 | 66.153 | 1.00 | 12.39 | DIC |
| ATOM | 757 | O | LEU | 100 | 40.458 | 45.100 | 64.941 | 1.00 | 12.90 | DIC |
| ATOM | 758 | N | TYR | 101 | 38.972 | 45.014 | 66.651 | 1.00 | 11.19 | DIC |
| ATOM | 759 | CA | TYR | 101 | 37.802 | 44.862 | 65.804 | 1.00 | 11.19 | DIC |
| ATOM | 760 | CB | TYR | 101 | 36.703 | 44.095 | 66.539 | 1.00 | 13.00 | DIC |
| ATOM | 761 | CG | TYR | 101 | 35.517 | 43.788 | 65.652 | 1.00 | 15.87 | DIC |
| ATOM | 762 | CD1 | TYR | 101 | 35.497 | 42.628 | 64.881 | 1.00 | 18.26 | DIC |
| ATOM | 763 | CE1 | TYR | 101 | 34.440 | 42.343 | 64.035 | 1.00 | 21.70 | DIC |
| ATOM | 764 | CD2 | TYR | 101 | 34.438 | 44.667 | 65.549 | 1.00 | 18.95 | DIC |
| ATOM | 765 | CE2 | TYR | 101 | 33.360 | 44.389 | 64.690 | 1.00 | 21.15 | DIC |
| ATOM | 766 | CZ | TYR | 101 | 33.376 | 43.219 | 63.942 | 1.00 | 21.56 | DIC |
| ATOM | 767 | OH | TYR | 101 | 32.328 | 42.882 | 63.115 | 1.00 | 26.24 | DIC |
| ATOM | 768 | C | TYR | 101 | 37.236 | 46.214 | 65.392 | 1.00 | 10.61 | DIC |
| ATOM | 769 | O | TYR | 101 | 37.076 | 47.108 | 66.228 | 1.00 | 9.84 | DIC |
| ATOM | 770 | N | LEU | 102 | 36.922 | 46.354 | 64.108 | 1.00 | 9.31 | DIC |
| ATOM | 771 | CA | LEU | 102 | 36.345 | 47.588 | 63.601 | 1.00 | 9.32 | DIC |
| ATOM | 772 | CB | LEU | 102 | 37.359 | 48.365 | 62.762 | 1.00 | 10.13 | DIC |
| ATOM | 773 | CG | LEU | 102 | 38.698 | 48.757 | 63.390 | 1.00 | 11.91 | DIC |
| ATOM | 774 | CD1 | LEU | 102 | 39.612 | 49.271 | 62.304 | 1.00 | 8.61 | DIC |
| ATOM | 775 | CD2 | LEU | 102 | 38.488 | 49.797 | 64.484 | 1.00 | 9.84 | DIC |
| ATOM | 776 | C | LEU | 102 | 35.135 | 47.298 | 62.725 | 1.00 | 9.76 | DIC |
| ATOM | 777 | O | LEU | 102 | 35.123 | 46.319 | 61.970 | 1.00 | 10.61 | DIC |
| ATOM | 778 | N | ASP | 103 | 34.119 | 48.149 | 62.834 | 1.00 | 8.81 | DIC |
| ATOM | 779 | CA | ASP | 103 | 32.938 | 48.028 | 61.993 | 1.00 | 10.01 | DIC |
| ATOM | 780 | CB | ASP | 103 | 31.848 | 48.969 | 62.449 | 1.00 | 8.25 | DIC |
| ATOM | 781 | CG | ASP | 103 | 30.891 | 48.371 | 63.428 | 1.00 | 9.78 | DIC |
| ATOM | 782 | OD1 | ASP | 103 | 29.964 | 49.092 | 63.841 | 1.00 | 8.38 | DIC |
| ATOM | 783 | OD2 | ASP | 103 | 31.050 | 47.196 | 63.787 | 1.00 | 8.21 | DIC |
| ATOM | 784 | C | ASP | 103 | 33.408 | 48.465 | 60.607 | 1.00 | 9.24 | DIC |
| ATOM | 785 | O | ASP | 103 | 34.485 | 49.041 | 60.463 | 1.00 | 7.13 | DIC |
| ATOM | 786 | N | ILE | 104 | 32.591 | 48.218 | 59.594 | 1.00 | 11.58 | DIC |
| ATOM | 787 | CA | ILE | 104 | 32.946 | 48.594 | 58.226 | 1.00 | 10.90 | DIC |
| ATOM | 788 | CB | ILE | 104 | 32.103 | 47.800 | 57.203 | 1.00 | 11.09 | DIC |
| ATOM | 789 | CG2 | ILE | 104 | 32.432 | 48.255 | 55.785 | 1.00 | 10.13 | DIC |
| ATOM | 790 | CG1 | ILE | 104 | 32.361 | 46.295 | 57.365 | 1.00 | 9.33 | DIC |
| ATOM | 791 | CD | ILE | 104 | 33.764 | 45.856 | 57.023 | 1.00 | 8.77 | DIC |
| ATOM | 792 | C | ILE | 104 | 32.732 | 50.085 | 57.980 | 1.00 | 11.23 | DIC |
| ATOM | 793 | O | ILE | 104 | 33.430 | 50.691 | 57.164 | 1.00 | 10.66 | DIC |
| ATOM | 794 | N | ASP | 105 | 31.764 | 50.667 | 58.687 | 1.00 | 10.09 | DIC |
| ATOM | 795 | CA | ASP | 105 | 31.439 | 52.085 | 58.545 | 1.00 | 10.37 | DIC |
| ATOM | 796 | CB | ASP | 105 | 29.959 | 52.325 | 58.896 | 1.00 | 9.81 | DIC |
| ATOM | 797 | CG | ASP | 105 | 29.608 | 51.900 | 60.318 | 1.00 | 7.95 | DIC |
| ATOM | 798 | OD1 | ASP | 105 | 30.413 | 51.181 | 60.955 | 1.00 | 10.48 | DIC |
| ATOM | 799 | OD2 | ASP | 105 | 28.510 | 52.275 | 60.799 | 1.00 | 8.90 | DIC |
| ATOM | 800 | C | ASP | 105 | 32.328 | 52.996 | 59.397 | 1.00 | 11.29 | DIC |
| ATOM | 801 | O | ASP | 105 | 31.838 | 53.871 | 60.121 | 1.00 | 10.81 | DIC |
| ATOM | 802 | N | VAL | 106 | 33.637 | 52.774 | 59.323 | 1.00 | 12.07 | DIC |
| ATOM | 803 | CA | VAL | 106 | 34.582 | 53.600 | 60.061 | 1.00 | 12.81 | DIC |
| ATOM | 804 | CB | VAL | 106 | 35.307 | 52.818 | 61.191 | 1.00 | 14.09 | DIC |
| ATOM | 805 | CG1 | VAL | 106 | 34.290 | 52.149 | 62.097 | 1.00 | 9.82 | DIC |
| ATOM | 806 | CG2 | VAL | 106 | 36.278 | 51.805 | 60.588 | 1.00 | 13.66 | DIC |
| ATOM | 807 | C | VAL | 106 | 35.641 | 54.127 | 59.106 | 1.00 | 13.02 | DIC |
| ATOM | 808 | O | VAL | 106 | 35.901 | 53.545 | 58.050 | 1.00 | 12.85 | DIC |
| ATOM | 809 | N | LEU | 107 | 36.241 | 55.242 | 59.490 | 1.00 | 14.12 | DIC |
| ATOM | 810 | CA | LEU | 107 | 37.288 | 55.875 | 58.710 | 1.00 | 14.17 | DIC |
| ATOM | 811 | CB | LEU | 107 | 36.743 | 57.141 | 58.050 | 1.00 | 15.58 | DIC |
| ATOM | 812 | CG | LEU | 107 | 36.665 | 57.238 | 56.522 | 1.00 | 18.69 | DIC |
| ATOM | 813 | CD1 | LEU | 107 | 36.107 | 55.963 | 55.914 | 1.00 | 19.47 | DIC |
| ATOM | 814 | CD2 | LEU | 107 | 35.800 | 58.438 | 56.155 | 1.00 | 18.85 | DIC |
| ATOM | 815 | C | LEU | 107 | 38.364 | 56.220 | 59.734 | 1.00 | 13.89 | DIC |
| ATOM | 816 | O | LEU | 107 | 38.165 | 57.089 | 60.581 | 1.00 | 13.18 | DIC |
| ATOM | 817 | N | VAL | 108 | 39.485 | 55.511 | 59.686 | 1.00 | 14.30 | DIC |
| ATOM | 818 | CA | VAL | 106 | 40.575 | 55.766 | 60.625 | 1.00 | 14.74 | DIC |
| ATOM | 819 | CB | VAL | 108 | 41.566 | 54.581 | 60.654 | 1.00 | 14.99 | DIC |
| ATOM | 820 | CG1 | VAL | 108 | 42.703 | 54.861 | 61.648 | 1.00 | 12.98 | DIC |
| ATOM | 821 | CG2 | VAL | 108 | 40.825 | 53.316 | 61.039 | 1.00 | 14.00 | DIC |
| ATOM | 822 | C | VAL | 108 | 41.281 | 57.027 | 60.159 | 1.00 | 15.87 | DIC |
| ATOM | 823 | O | VAL | 108 | 41.740 | 57.093 | 59.015 | 1.00 | 17.65 | DIC |
| ATOM | 824 | N | ARG | 109 | 41.350 | 58.034 | 61.029 | 1.00 | 17.02 | DIC |
| ATOM | 825 | CA | ARG | 109 | 41.981 | 59.305 | 60.668 | 1.00 | 17.83 | DIC |
| ATOM | 826 | CB | ARG | 109 | 41.011 | 60.467 | 60.903 | 1.00 | 20.81 | DIC |
| ATOM | 827 | CG | ARG | 109 | 39.553 | 60.066 | 60.959 | 1.00 | 25.33 | DIC |
| ATOM | 828 | CD | ARG | 109 | 38.684 | 61.042 | 60.198 | 1.00 | 27.87 | DIC |
| ATOM | 829 | NE | ARG | 109 | 39.003 | 62.443 | 60.463 | 1.00 | 30.93 | DIC |
| ATOM | 830 | CZ | ARG | 109 | 38.574 | 63.450 | 59.702 | 1.00 | 31.43 | DIC |
| ATOM | 831 | NH1 | ARG | 109 | 38.902 | 64.702 | 59.994 | 1.00 | 29.96 | DIC |
| ATOM | 832 | NH2 | ARG | 109 | 37.817 | 63.198 | 58.640 | 1.00 | 29.66 | DIC |
| ATOM | 833 | C | ARG | 109 | 43.277 | 59.603 | 61.413 | 1.00 | 18.31 | DIC |
| ATOM | 834 | O | ARG | 109 | 43.945 | 60.597 | 61.127 | 1.00 | 18.37 | DIC |
| ATOM | 835 | N | ASP | 110 | 43.614 | 58.767 | 62.388 | 1.00 | 15.42 | DIC |
| ATOM | 836 | CA | ASP | 110 | 44.846 | 58.952 | 63.143 | 1.00 | 16.34 | DIC |
| ATOM | 837 | CB | ASP | 110 | 44.627 | 59.926 | 64.312 | 1.00 | 18.08 | DIC |
| ATOM | 838 | CG | ASP | 110 | 45.936 | 60.528 | 64.830 | 1.00 | 22.13 | DIC |
| ATOM | 839 | OD1 | ASP | 110 | 46.857 | 60.743 | 64.015 | 1.00 | 21.78 | DIC |
| ATOM | 840 | OD2 | ASP | 110 | 46.040 | 60.802 | 66.045 | 1.00 | 23.13 | DIC |
| ATOM | 841 | C | ASP | 110 | 45.303 | 57.590 | 63.653 | 1.00 | 14.85 | DIC |
| ATOM | 842 | O | ASP | 110 | 44.523 | 56.645 | 63.698 | 1.00 | 13.20 | DIC |
| ATOM | 843 | N | ARG | 111 | 46.571 | 57.499 | 64.028 | 1.00 | 15.06 | DIC |
| ATOM | 844 | CA | ARG | 111 | 47.146 | 56.255 | 64.510 | 1.00 | 15.16 | DIC |
| ATOM | 845 | CB | ARG | 111 | 48.553 | 56.524 | 65.030 | 1.00 | 18.81 | DIC |
| ATOM | 846 | CG | ARG | 111 | 49.217 | 55.331 | 65.622 | 1.00 | 18.92 | DIC |
| ATOM | 847 | CD | ARG | 111 | 49.846 | 55.728 | 66.938 | 1.00 | 31.56 | DIC |
| ATOM | 848 | NE | ARG | 111 | 50.989 | 56.627 | 66.821 | 1.00 | 33.85 | DIC |
| ATOM | 849 | CZ | ARG | 111 | 51.668 | 57.080 | 67.869 | 1.00 | 36.04 | DIC |
| ATOM | 850 | NH1 | ARG | 111 | 51.307 | 56.709 | 69.092 | 1.00 | 34.31 | DIC |
| ATOM | 851 | NH2 | ARG | 111 | 52.706 | 57.897 | 67.699 | 1.00 | 37.36 | DIC |
| ATOM | 852 | C | ARG | 111 | 46.315 | 55.573 | 65.596 | 1.00 | 13.47 | DIC |
| ATOM | 853 | O | ARG | 111 | 45.751 | 56.226 | 66.473 | 1.00 | 10.45 | DIC |
| ATOM | 854 | N | LEU | 112 | 46.268 | 54.248 | 65.534 | 1.00 | 11.22 | DIC |
| ATOM | 855 | CA | LEU | 112 | 45.520 | 53.445 | 66.493 | 1.00 | 12.34 | DIC |
| ATOM | 856 | CB | LEU | 112 | 44.797 | 52.318 | 65.747 | 1.00 | 13.02 | DIC |
| ATOM | 857 | CG | LEU | 112 | 43.282 | 52.409 | 65.491 | 1.00 | 17.78 | DIC |
| ATOM | 858 | CD1 | LEU | 112 | 42.810 | 53.847 | 65.418 | 1.00 | 15.27 | DIC |
| ATOM | 859 | CD2 | LEU | 112 | 42.951 | 51.643 | 64.218 | 1.00 | 14.01 | DIC |
| ATOM | 860 | C | LEU | 112 | 46.455 | 52.853 | 67.545 | 1.00 | 11.08 | DIC |
| ATOM | 861 | O | LEU | 112 | 46.013 | 52.187 | 68.481 | 1.00 | 10.55 | DIC |
| ATOM | 862 | N | THR | 113 | 47.749 | 53.105 | 67.397 | 1.00 | 10.99 | DIC |
| ATOM | 863 | CA | THR | 113 | 48.735 | 52.555 | 68.322 | 1.00 | 13.17 | DIC |
| ATOM | 864 | CB | THR | 113 | 50.146 | 53.084 | 67.987 | 1.00 | 14.67 | DIC |
| ATOM | 865 | OG1 | THR | 113 | 50.429 | 52.810 | 66.607 | 1.00 | 15.24 | DIC |
| ATOM | 866 | CG2 | THR | 113 | 51.203 | 52.421 | 68.863 | 1.00 | 14.83 | DIC |
| ATOM | 867 | C | THR | 113 | 48.423 | 52.788 | 69.802 | 1.00 | 13.22 | DIC |
| ATOM | 868 | O | THR | 113 | 48.550 | 51.869 | 70.616 | 1.00 | 13.60 | DIC |
| ATOM | 869 | N | PRO | 114 | 48.009 | 54.013 | 70.177 | 1.00 | 13.02 | DIC |
| ATOM | 870 | CD | PRO | 114 | 47.875 | 55.254 | 69.398 | 1.00 | 12.48 | DIC |
| ATOM | 871 | CA | PRO | 114 | 47.701 | 54.250 | 71.593 | 1.00 | 13.64 | DIC |
| ATOM | 872 | CB | PRO | 114 | 47.182 | 55.686 | 71.600 | 1.00 | 14.06 | DIC |
| ATOM | 873 | CG | PRO | 114 | 47.955 | 56.321 | 70.468 | 1.00 | 14.40 | DIC |
| ATOM | 874 | C | PRO | 114 | 46.651 | 53.268 | 72.098 | 1.00 | 12.02 | DIC |
| ATOM | 875 | O | PRO | 114 | 46.751 | 52.759 | 73.211 | 1.00 | 13.45 | DIC |
| ATOM | 876 | N | LEU | 115 | 45.643 | 53.003 | 71.272 | 1.00 | 11.75 | DIC |
| ATOM | 877 | CA | LEU | 115 | 44.582 | 52.076 | 71.659 | 1.00 | 11.54 | DIC |
| ATOM | 878 | CB | LEU | 115 | 43.389 | 52.193 | 70.696 | 1.00 | 10.01 | DIC |
| ATOM | 879 | CG | LEU | 115 | 42.236 | 51.214 | 70.936 | 1.00 | 11.01 | DIC |
| ATOM | 880 | CD1 | LEU | 115 | 41.718 | 51.381 | 72.358 | 1.00 | 10.94 | DIC |
| ATOM | 881 | CD2 | LEU | 115 | 41.116 | 51.460 | 69.921 | 1.00 | 11.28 | DIC |
| ATOM | 882 | C | LEU | 115 | 45.131 | 50.656 | 71.656 | 1.00 | 10.96 | DIC |
| ATOM | 883 | O | LEU | 115 | 44.949 | 49.905 | 72.613 | 1.00 | 12.79 | DIC |
| ATOM | 884 | N | TRP | 116 | 45.817 | 50.301 | 70.578 | 1.00 | 11.17 | DIC |
| ATOM | 885 | CA | TRP | 116 | 46.410 | 48.977 | 70.443 | 1.00 | 12.32 | DIC |
| ATOM | 886 | CB | TRP | 116 | 47.161 | 48.880 | 69.113 | 1.00 | 11.64 | DIC |
| ATOM | 887 | CG | TRP | 116 | 47.885 | 47.579 | 68.933 | 1.00 | 14.42 | DIC |
| ATOM | 888 | CD2 | TRP | 116 | 47.304 | 46.314 | 68.595 | 1.00 | 12.78 | DIC |
| ATOM | 889 | CE2 | TRP | 116 | 48.352 | 45.365 | 68.570 | 1.00 | 13.04 | DIC |
| ATOM | 890 | CE3 | TRP | 116 | 45.996 | 45.887 | 68.312 | 1.00 | 13.68 | DIC |
| ATOM | 891 | CD1 | TRP | 116 | 49.222 | 47.355 | 69.096 | 1.00 | 14.44 | DIC |
| ATOM | 892 | NE1 | TRP | 116 | 49.511 | 46.027 | 68.878 | 1.00 | 14.14 | DIC |
| ATOM | 893 | CZ2 | TRP | 116 | 48.137 | 44.014 | 68.272 | 1.00 | 12.47 | DIC |
| ATOM | 894 | CZ3 | TRP | 116 | 45.782 | 44.539 | 68.014 | 1.00 | 13.84 | DIC |
| ATOM | 895 | CH2 | TRP | 116 | 46.851 | 43.621 | 67.997 | 1.00 | 13.37 | DIC |
| ATOM | 896 | C | TRP | 116 | 47.356 | 48.645 | 71.599 | 1.00 | 12.95 | DIC |
| ATOM | 897 | O | TRP | 116 | 47.404 | 47.504 | 72.056 | 1.00 | 13.17 | DIC |
| ATOM | 898 | N | ASP | 117 | 48.092 | 49.641 | 72.082 | 1.00 | 14.18 | DIC |
| ATOM | 899 | CA | ASP | 117 | 49.038 | 49.416 | 73.173 | 1.00 | 13.65 | DIC |
| ATOM | 900 | CB | ASP | 117 | 50.122 | 50.502 | 73.180 | 1.00 | 12.84 | DIC |
| ATOM | 901 | CG | ASP | 117 | 51.066 | 50.395 | 71.998 | 1.00 | 15.20 | DIC |
| ATOM | 902 | OD1 | ASP | 117 | 51.218 | 49.285 | 71.446 | 1.00 | 11.63 | DIC |
| ATOM | 903 | OD2 | ASP | 117 | 51.668 | 51.423 | 71.631 | 1.00 | 16.23 | DIC |
| ATOM | 904 | C | ASP | 117 | 48.403 | 49.352 | 74.554 | 1.00 | 14.05 | DIC |
| ATOM | 905 | O | ASP | 117 | 49.089 | 49.088 | 75.546 | 1.00 | 13.05 | DIC |
| ATOM | 906 | N | THR | 118 | 47.099 | 49.593 | 74.624 | 1.00 | 14.89 | DIC |
| ATOM | 907 | CA | THR | 118 | 46.401 | 49.572 | 75.903 | 1.00 | 15.50 | DIC |
| ATOM | 908 | CB | THR | 118 | 44.935 | 50.017 | 75.736 | 1.00 | 16.17 | DIC |
| ATOM | 909 | OG1 | THR | 118 | 44.902 | 51.379 | 75.272 | 1.00 | 16.94 | DIC |
| ATOM | 910 | CG2 | THR | 118 | 44.197 | 49.922 | 77.066 | 1.00 | 16.68 | DIC |
| ATOM | 911 | C | THR | 118 | 46.440 | 48.189 | 76.542 | 1.00 | 16.76 | DIC |
| ATOM | 912 | O | THR | 118 | 46.188 | 47.181 | 75.885 | 1.00 | 16.13 | DIC |
| ATOM | 913 | N | ASP | 119 | 46.769 | 48.140 | 77.827 | 1.00 | 17.70 | DIC |
| ATOM | 914 | CA | ASP | 119 | 46.816 | 46.861 | 78.531 | 1.00 | 19.90 | DIC |
| ATOM | 915 | CB | ASP | 119 | 47.841 | 46.926 | 79.669 | 1.00 | 22.37 | DIC |
| ATOM | 916 | CG | ASP | 119 | 47.885 | 45.653 | 80.501 | 1.00 | 24.15 | DIC |
| ATOM | 917 | OD1 | ASP | 119 | 47.539 | 44.567 | 79.989 | 1.00 | 22.37 | DIC |
| ATOM | 918 | OD2 | ASP | 119 | 48.289 | 45.744 | 81.678 | 1.00 | 26.50 | DIC |
| ATOM | 919 | C | ASP | 119 | 45.422 | 46.543 | 79.066 | 1.00 | 19.45 | DIC |
| ATOM | 920 | O | ASP | 119 | 44.899 | 47.250 | 79.931 | 1.00 | 20.25 | DIC |
| ATOM | 921 | N | LEU | 120 | 44.823 | 45.484 | 78.528 | 1.00 | 19.08 | DIC |
| ATOM | 922 | CA | LEU | 120 | 43.480 | 45.051 | 78.916 | 1.00 | 19.39 | DIC |
| ATOM | 923 | CB | LEU | 120 | 42.906 | 44.117 | 77.846 | 1.00 | 16.25 | DIC |
| ATOM | 924 | CG | LEU | 120 | 42.192 | 44.700 | 76.623 | 1.00 | 18.43 | DIC |
| ATOM | 925 | CD1 | LEU | 120 | 42.797 | 46.005 | 76.207 | 1.00 | 19.56 | DIC |
| ATOM | 926 | CD2 | LEU | 120 | 42.246 | 43.680 | 75.502 | 1.00 | 14.56 | DIC |
| ATOM | 927 | C | LEU | 120 | 43.413 | 44.342 | 80.264 | 1.00 | 19.79 | DIC |
| ATOM | 928 | O | LEU | 120 | 42.337 | 44.230 | 80.852 | 1.00 | 21.22 | DIC |
| ATOM | 929 | N | GLY | 121 | 44.552 | 43.861 | 80.754 | 1.00 | 20.41 | DIC |
| ATOM | 930 | CA | GLY | 121 | 44.539 | 43.156 | 82.024 | 1.00 | 20.26 | DIC |
| ATOM | 931 | C | GLY | 121 | 43.648 | 41.934 | 81.880 | 1.00 | 20.32 | DIC |
| ATOM | 932 | O | GLY | 121 | 43.738 | 41.213 | 80.880 | 1.00 | 18.77 | DIC |
| ATOM | 933 | N | ASN | 122 | 42.778 | 41.695 | 82.855 | 1.00 | 20.30 | DIC |
| ATOM | 934 | CA | ASN | 122 | 41.891 | 40.540 | 82.772 | 1.00 | 21.26 | DIC |
| ATOM | 935 | CB | ASN | 122 | 41.778 | 39.852 | 84.135 | 1.00 | 23.94 | DIC |
| ATOM | 936 | CG | ASN | 122 | 41.067 | 38.510 | 84.054 | 1.00 | 27.72 | DIC |
| ATOM | 937 | OD1 | ASN | 122 | 41.427 | 37.643 | 83.247 | 1.00 | 29.43 | DIC |
| ATOM | 938 | ND2 | ASN | 122 | 40.056 | 38.329 | 84.893 | 1.00 | 29.68 | DIC |
| ATOM | 939 | C | ASN | 122 | 40.508 | 40.940 | 82.254 | 1.00 | 20.06 | DIC |
| ATOM | 940 | O | ASN | 122 | 39.545 | 40.173 | 82.356 | 1.00 | 19.14 | DIC |
| ATOM | 941 | N | ASN | 123 | 40.421 | 42.150 | 81.704 | 1.00 | 18.17 | DIC |
| ATOM | 942 | CA | ASN | 123 | 39.173 | 42.656 | 81.137 | 1.00 | 17.70 | DIC |
| ATOM | 943 | CB | ASN | 123 | 39.278 | 44.158 | 80.810 | 1.00 | 19.01 | DIC |
| ATOM | 944 | CG | ASN | 123 | 39.293 | 45.042 | 82.053 | 1.00 | 20.14 | DIC |
| ATOM | 945 | OD1 | ASN | 123 | 40.257 | 45.769 | 82.303 | 1.00 | 19.33 | DIC |
| ATOM | 946 | ND2 | ASN | 123 | 38.218 | 44.990 | 82.829 | 1.00 | 17.24 | DIC |
| ATOM | 947 | C | ASN | 123 | 38.894 | 41.891 | 79.842 | 1.00 | 17.42 | DIC |
| ATOM | 948 | O | ASN | 123 | 39.818 | 41.387 | 79.194 | 1.00 | 16.53 | DIC |
| ATOM | 949 | N | TRP | 124 | 37.619 | 41.823 | 79.469 | 1.00 | 16.21 | DIC |
| ATOM | 950 | CA | TRP | 124 | 37.191 | 41.136 | 78.260 | 1.00 | 13.67 | DIC |
| ATOM | 951 | CB | TRP | 124 | 35.675 | 40.980 | 78.246 | 1.00 | 13.09 | DIC |
| ATOM | 952 | CG | TRP | 124 | 35.140 | 40.061 | 79.278 | 1.00 | 14.06 | DIC |
| ATOM | 953 | CD2 | TRP | 124 | 35.114 | 38.631 | 79.212 | 1.00 | 13.39 | DIC |
| ATOM | 954 | CE2 | TRP | 124 | 34.475 | 38.169 | 80.389 | 1.00 | 14.28 | DIC |
| ATOM | 955 | CE3 | TRP | 124 | 35.566 | 37.694 | 78.273 | 1.00 | 14.36 | DIC |
| ATOM | 956 | CD1 | TRP | 124 | 34.535 | 40.406 | 80.461 | 1.00 | 11.88 | DIC |
| ATOM | 957 | NE1 | TRP | 124 | 34.131 | 39.272 | 81.132 | 1.00 | 12.38 | DIC |
| ATOM | 958 | CZ2 | TRP | 124 | 34.278 | 36.808 | 80.649 | 1.00 | 13.77 | DIC |
| ATOM | 959 | CZ3 | TRP | 124 | 35.370 | 36.338 | 78.534 | 1.00 | 15.53 | DIC |
| ATOM | 960 | CH2 | TRP | 124 | 34.730 | 35.911 | 79.714 | 1.00 | 15.41 | DIC |
| ATOM | 961 | C | TRP | 124 | 37.591 | 41.898 | 77.006 | 1.00 | 13.43 | DIC |
| ATOM | 962 | O | TRP | 124 | 37.899 | 41.303 | 75.979 | 1.00 | 12.54 | DIC |
| ATOM | 963 | N | LEU | 125 | 37.570 | 43.221 | 77.102 | 1.00 | 11.84 | DIC |
| ATOM | 964 | CA | LEU | 125 | 37.897 | 44.062 | 75.970 | 1.00 | 11.77 | DIC |
| ATOM | 965 | CB | LEU | 125 | 36.845 | 43.874 | 74.861 | 1.00 | 10.08 | DIC |
| ATOM | 966 | CG | LEU | 125 | 35.361 | 43.957 | 75.259 | 1.00 | 10.79 | DIC |
| ATOM | 967 | CD1 | LEU | 125 | 35.006 | 45.378 | 75.672 | 1.00 | 14.40 | DIC |
| ATOM | 968 | CD2 | LEU | 125 | 34.486 | 43.530 | 74.086 | 1.00 | 11.76 | DIC |
| ATOM | 969 | C | LEU | 125 | 37.958 | 45.524 | 76.368 | 1.00 | 11.36 | DIC |
| ATOM | 970 | O | LEU | 125 | 37.614 | 45.904 | 77.488 | 1.00 | 12.15 | DIC |
| ATOM | 971 | N | GLY | 126 | 38.419 | 46.336 | 75.432 | 1.00 | 11.25 | DIC |
| ATOM | 972 | CA | GLY | 126 | 38.492 | 47.763 | 75.651 | 1.00 | 11.38 | DIC |
| ATOM | 973 | C | GLY | 126 | 37.584 | 48.350 | 74.588 | 1.00 | 11.99 | DIC |
| ATOM | 974 | O | GLY | 126 | 37.600 | 47.893 | 73.435 | 1.00 | 11.61 | DIC |
| ATOM | 975 | N | ALA | 127 | 36.779 | 49.338 | 74.962 | 1.00 | 10.89 | DIC |
| ATOM | 976 | CA | ALA | 127 | 35.868 | 49.967 | 74.013 | 1.00 | 10.27 | DIC |
| ATOM | 977 | CB | ALA | 127 | 34.608 | 49.121 | 73.871 | 1.00 | 9.81 | DIC |
| ATOM | 978 | C | ALA | 127 | 35.499 | 51.383 | 74.444 | 1.00 | 11.20 | DIC |
| ATOM | 979 | O | ALA | 127 | 35.663 | 51.749 | 75.612 | 1.00 | 8.75 | DIC |
| ATOM | 980 | N | SER | 128 | 35.017 | 52.185 | 73.496 | 1.00 | 10.96 | DIC |
| ATOM | 981 | CA | SER | 128 | 34.622 | 53.551 | 73.811 | 1.00 | 12.41 | DIC |
| ATOM | 982 | CB | SER | 128 | 34.844 | 54.480 | 72.610 | 1.00 | 11.48 | DIC |
| ATOM | 983 | OG | SER | 128 | 36.228 | 54.600 | 72.303 | 1.00 | 12.36 | DIC |
| ATOM | 984 | C | SER | 128 | 33.153 | 53.563 | 74.220 | 1.00 | 13.04 | DIC |
| ATOM | 985 | O | SER | 128 | 32.362 | 52.717 | 73.783 | 1.00 | 11.02 | DIC |
| ATOM | 986 | N | ILE | 129 | 32.800 | 54.535 | 75.055 | 1.00 | 12.60 | DIC |
| ATOM | 987 | CA | ILE | 129 | 31.441 | 54.678 | 75.568 | 1.00 | 14.09 | DIC |
| ATOM | 988 | CB | ILE | 129 | 31.433 | 55.609 | 76.816 | 1.00 | 14.78 | DIC |
| ATOM | 989 | CG2 | ILE | 129 | 30.009 | 55.993 | 77.195 | 1.00 | 15.61 | DIC |
| ATOM | 990 | CG1 | ILE | 129 | 32.154 | 54.916 | 77.973 | 1.00 | 16.10 | DIC |
| ATOM | 991 | CD | ILE | 129 | 32.334 | 55.785 | 79.199 | 1.00 | 16.05 | DIC |
| ATOM | 992 | C | ILE | 129 | 30.484 | 55.232 | 74.519 | 1.00 | 14.40 | DIC |
| ATOM | 993 | O | ILE | 129 | 30.841 | 56.112 | 73.736 | 1.00 | 12.33 | DIC |
| ATOM | 994 | N | ASP | 130 | 29.263 | 54.708 | 74.513 | 1.00 | 14.40 | DIC |
| ATOM | 995 | CA | ASP | 130 | 28.253 | 55.155 | 73.571 | 1.00 | 14.93 | DIC |
| ATOM | 996 | CB | ASP | 130 | 27.396 | 53.972 | 73.127 | 1.00 | 13.65 | DIC |
| ATOM | 997 | CG | ASP | 130 | 26.533 | 54.305 | 71.936 | 1.00 | 12.84 | DIC |
| ATOM | 998 | OD1 | ASP | 130 | 25.787 | 55.315 | 71.985 | 1.00 | 15.16 | DIC |
| ATOM | 999 | OD2 | ASP | 130 | 26.605 | 53.556 | 70.953 | 1.00 | 12.26 | DIC |
| ATOM | 1000 | C | ASP | 130 | 27.359 | 56.209 | 74.228 | 1.00 | 14.81 | DIC |
| ATOM | 1001 | O | ASP | 130 | 26.458 | 55.868 | 74.991 | 1.00 | 15.34 | DIC |
| ATOM | 1002 | N | LEU | 131 | 27.605 | 57.482 | 73.932 | 1.00 | 15.23 | DIC |
| ATOM | 1003 | CA | LEU | 131 | 26.811 | 58.576 | 74.509 | 1.00 | 15.08 | DIC |
| ATOM | 1004 | CB | LEU | 131 | 27.344 | 59.936 | 74.049 | 1.00 | 15.03 | DIC |
| ATOM | 1005 | CG | LEU | 131 | 28.638 | 60.445 | 74.680 | 1.00 | 18.24 | DIC |
| ATOM | 1006 | CD1 | LEU | 131 | 29.710 | 59.364 | 74.623 | 1.00 | 20.00 | DIC |
| ATOM | 1007 | CD2 | LEU | 131 | 29.090 | 61.700 | 73.947 | 1.00 | 16.98 | DIC |
| ATOM | 1008 | C | LEU | 131 | 25.339 | 58.497 | 74.139 | 1.00 | 14.84 | DIC |
| ATOM | 1009 | O | LEU | 131 | 24.466 | 58.863 | 74.933 | 1.00 | 15.29 | DIC |
| ATOM | 1010 | N | PHE | 132 | 25.068 | 58.041 | 72.922 | 1.00 | 13.40 | DIC |
| ATOM | 1011 | CA | PHE | 132 | 23.700 | 57.930 | 72.440 | 1.00 | 15.19 | DIC |
| ATOM | 1012 | CB | PHE | 132 | 23.698 | 57.464 | 70.976 | 1.00 | 16.45 | DIC |
| ATOM | 1013 | CG | PHE | 132 | 22.325 | 57.299 | 70.391 | 1.00 | 18.23 | DIC |
| ATOM | 1014 | CD1 | PHE | 132 | 21.657 | 56.079 | 70.480 | 1.00 | 19.03 | DIC |
| ATOM | 1015 | CD2 | PHE | 132 | 21.680 | 58.371 | 69.793 | 1.00 | 19.73 | DIC |
| ATOM | 1016 | CE1 | PHE | 132 | 20.365 | 55.931 | 69.984 | 1.00 | 18.68 | DIC |
| ATOM | 1017 | CE2 | PHE | 132 | 20.381 | 58.235 | 69.292 | 1.00 | 21.22 | DIC |
| ATOM | 1018 | CZ | PHE | 132 | 19.723 | 57.010 | 69.389 | 1.00 | 19.92 | DIC |
| ATOM | 1019 | C | PHE | 132 | 22.898 | 56.971 | 73.320 | 1.00 | 15.39 | DIC |
| ATOM | 1020 | O | PHE | 132 | 21.812 | 57.316 | 73.791 | 1.00 | 17.07 | DIC |
| ATOM | 1021 | N | VAL | 133 | 23.446 | 55.781 | 73.553 | 1.00 | 12.91 | DIC |
| ATOM | 1022 | CA | VAL | 133 | 22.771 | 54.783 | 74.375 | 1.00 | 14.30 | DIC |
| ATOM | 1023 | CB | VAL | 133 | 23.439 | 53.399 | 74.241 | 1.00 | 13.72 | DIC |
| ATOM | 1024 | CG1 | VAL | 133 | 22.727 | 52.386 | 75.138 | 1.00 | 14.53 | DIC |
| ATOM | 1025 | CG2 | VAL | 133 | 23.375 | 52.939 | 72.794 | 1.00 | 13.31 | DIC |
| ATOM | 1026 | C | VAL | 133 | 22.741 | 55.184 | 75.845 | 1.00 | 15.10 | DIC |
| ATOM | 1027 | O | VAL | 133 | 21.710 | 55.049 | 76.503 | 1.00 | 15.38 | DIC |
| ATOM | 1028 | N | GLU | 134 | 23.867 | 55.682 | 76.352 | 1.00 | 16.89 | DIC |
| ATOM | 1029 | CA | GLU | 134 | 23.951 | 56.121 | 77.746 | 1.00 | 18.11 | DIC |
| ATOM | 1030 | CB | GLU | 134 | 25.334 | 56.708 | 78.058 | 1.00 | 17.79 | DIC |
| ATOM | 1031 | CG | GLU | 134 | 26.358 | 55.714 | 78.580 | 1.00 | 18.79 | DIC |
| ATOM | 1032 | CD | GLU | 134 | 25.943 | 55.071 | 79.893 | 1.00 | 15.84 | DIC |
| ATOM | 1033 | OE1 | GLU | 134 | 25.534 | 55.790 | 80.828 | 1.00 | 18.93 | DIC |
| ATOM | 1034 | OE2 | GLU | 134 | 26.035 | 53.838 | 79.993 | 1.00 | 16.91 | DIC |
| ATOM | 1035 | C | GLU | 134 | 22.904 | 57.169 | 78.092 | 1.00 | 18.74 | DIC |
| ATOM | 1036 | O | GLU | 134 | 22.397 | 57.196 | 79.207 | 1.00 | 19.62 | DIC |
| ATOM | 1037 | N | ARG | 135 | 22.583 | 58.054 | 77.163 | 1.00 | 20.18 | DIC |
| ATOM | 1038 | CA | ARG | 135 | 21.592 | 59.061 | 77.501 | 1.00 | 21.49 | DIC |
| ATOM | 1039 | CB | ARG | 135 | 21.898 | 60.402 | 76.820 | 1.00 | 22.17 | DIC |
| ATOM | 1040 | CG | ARG | 135 | 21.852 | 60.441 | 75.309 | 1.00 | 26.54 | DIC |
| ATOM | 1041 | CD | ARG | 135 | 22.447 | 61.769 | 74.834 | 1.00 | 30.72 | DIC |
| ATOM | 1042 | NE | ARG | 135 | 21.803 | 62.903 | 75.492 | 1.00 | 33.69 | DIC |
| ATOM | 1043 | CZ | ARG | 135 | 20.912 | 63.708 | 74.916 | 1.00 | 35.46 | DIC |
| ATOM | 1044 | NH1 | ARG | 135 | 20.380 | 64.705 | 75.612 | 1.00 | 36.28 | DIC |
| ATOM | 1045 | NH2 | ARG | 135 | 20.568 | 63.535 | 73.646 | 1.00 | 33.44 | DIC |
| ATOM | 1046 | C | ARG | 135 | 20.176 | 58.599 | 77.196 | 1.00 | 21.50 | DIC |
| ATOM | 1047 | O | ARG | 135 | 19.231 | 59.380 | 77.266 | 1.00 | 21.59 | DIC |
| ATOM | 1048 | N | GLN | 136 | 20.021 | 57.321 | 76.867 | 1.00 | 20.53 | DIC |
| ATOM | 1049 | CA | GLN | 136 | 18.689 | 56.790 | 76.600 | 1.00 | 21.69 | DIC |
| ATOM | 1050 | CB | GLN | 136 | 18.767 | 55.590 | 75.653 | 1.00 | 22.18 | DIC |
| ATOM | 1051 | CG | GLN | 136 | 17.408 | 55.118 | 75.147 | 1.00 | 24.69 | DIC |
| ATOM | 1052 | CD | GLN | 136 | 17.487 | 53.951 | 74.173 | 1.00 | 27.02 | DIC |
| ATOM | 1053 | OE1 | GLN | 136 | 18.419 | 53.858 | 73.384 | 1.00 | 26.51 | DIC |
| ATOM | 1054 | NE2 | GLN | 136 | 16.491 | 53.067 | 74.216 | 1.00 | 26.50 | DIC |
| ATOM | 1055 | C | GLN | 136 | 18.067 | 56.361 | 77.938 | 1.00 | 23.33 | DIC |
| ATOM | 1056 | O | GLN | 136 | 18.334 | 55.271 | 78.431 | 1.00 | 21.46 | DIC |
| ATOM | 1057 | N | GLU | 137 | 17.241 | 57.237 | 78.510 | 1.00 | 23.90 | DIC |
| ATOM | 1058 | CA | GLU | 137 | 16.549 | 57.028 | 79.789 | 1.00 | 26.86 | DIC |
| ATOM | 1059 | CB | GLU | 137 | 15.233 | 57.832 | 79.846 | 1.00 | 30.23 | DIC |
| ATOM | 1060 | CG | GLU | 137 | 15.271 | 59.120 | 80.663 | 1.00 | 35.53 | DIC |
| ATOM | 1061 | CD | GLU | 137 | 16.227 | 59.028 | 81.844 | 1.00 | 38.87 | DIC |
| ATOM | 1062 | OE1 | GLU | 137 | 16.443 | 57.933 | 82.435 | 1.00 | 40.95 | DIC |
| ATOM | 1063 | OE2 | GLU | 137 | 16.785 | 60.090 | 82.175 | 1.00 | 41.61 | DIC |
| ATOM | 1064 | C | GLU | 137 | 16.185 | 55.598 | 80.087 | 1.00 | 24.31 | DIC |
| ATOM | 1065 | O | GLU | 137 | 15.407 | 54.984 | 79.365 | 1.00 | 25.87 | DIC |
| ATOM | 1066 | N | GLY | 138 | 16.766 | 55.070 | 81.149 | 1.00 | 23.93 | DIC |
| ATOM | 1067 | CA | GLY | 138 | 16.451 | 53.729 | 81.594 | 1.00 | 22.33 | DIC |
| ATOM | 1068 | C | GLY | 138 | 16.756 | 52.535 | 80.724 | 1.00 | 21.12 | DIC |
| ATOM | 1069 | O | GLY | 138 | 16.509 | 51.410 | 81.164 | 1.00 | 22.81 | DIC |
| ATOM | 1070 | N | TYR | 139 | 17.289 | 52.734 | 79.526 | 1.00 | 18.91 | DIC |
| ATOM | 1071 | CA | TYR | 139 | 17.555 | 51.583 | 78.676 | 1.00 | 17.98 | DIC |
| ATOM | 1072 | CB | TYR | 139 | 18.002 | 52.012 | 77.282 | 1.00 | 17.34 | DIC |
| ATOM | 1073 | CG | TYR | 139 | 18.291 | 50.799 | 76.400 | 1.00 | 17.33 | DIC |
| ATOM | 1074 | CD1 | TYR | 139 | 17.254 | 49.968 | 75.965 | 1.00 | 18.26 | DIC |
| ATOM | 1075 | CE1 | TYR | 139 | 17.510 | 48.815 | 75.222 | 1.00 | 16.56 | DIC |
| ATOM | 1076 | CD2 | TYR | 139 | 19.602 | 50.442 | 76.062 | 1.00 | 15.79 | DIC |
| ATOM | 1077 | CE2 | TYR | 139 | 19.866 | 49.284 | 75.312 | 1.00 | 15.54 | DIC |
| ATOM | 1078 | CZ | TYR | 139 | 18.813 | 48.480 | 74.901 | 1.00 | 16.42 | DIC |
| ATOM | 1079 | OH | TYR | 139 | 19.045 | 47.340 | 74.165 | 1.00 | 15.74 | DIC |
| ATOM | 1080 | C | TYR | 139 | 18.584 | 50.596 | 79.212 | 1.00 | 17.11 | DIC |
| ATOM | 1081 | O | TYR | 139 | 18.348 | 49.386 | 79.242 | 1.00 | 17.36 | DIC |
| ATOM | 1082 | N | LYS | 140 | 19.744 | 51.130 | 79.571 | 1.00 | 15.75 | DIC |
| ATOM | 1083 | CA | LYS | 140 | 20.865 | 50.365 | 80.083 | 1.00 | 16.81 | DIC |
| ATOM | 1084 | CB | LYS | 140 | 21.894 | 51.354 | 80.635 | 1.00 | 19.79 | DIC |
| ATOM | 1085 | CG | LYS | 140 | 23.223 | 50.771 | 80.995 | 1.00 | 23.22 | DIC |
| ATOM | 1086 | CD | LYS | 140 | 24.253 | 51.863 | 81.267 | 1.00 | 21.44 | DIC |
| ATOM | 1087 | CE | LYS | 140 | 23.904 | 52.707 | 82.480 | 1.00 | 23.50 | DIC |
| ATOM | 1088 | NZ | LYS | 140 | 25.043 | 53.584 | 82.849 | 1.00 | 22.17 | DIC |
| ATOM | 1089 | C | LYS | 140 | 20.404 | 49.388 | 81.162 | 1.00 | 17.13 | DIC |
| ATOM | 1090 | O | LYS | 140 | 20.812 | 48.226 | 81.180 | 1.00 | 14.56 | DIC |
| ATOM | 1091 | N | GLN | 141 | 19.540 | 49.862 | 82.053 | 1.00 | 16.81 | DIC |
| ATOM | 1092 | CA | GLN | 141 | 19.027 | 49.029 | 83.130 | 1.00 | 18.98 | DIC |
| ATOM | 1093 | CB | GLN | 141 | 18.259 | 49.896 | 84.137 | 1.00 | 19.80 | DIC |
| ATOM | 1094 | CG | GLN | 141 | 19.152 | 50.846 | 84.948 | 1.00 | 18.45 | DIC |
| ATOM | 1095 | CD | GLN | 141 | 19.602 | 52.091 | 84.178 | 1.00 | 20.81 | DIC |
| ATOM | 1096 | OE1 | GLN | 141 | 19.304 | 52.258 | 82.994 | 1.00 | 19.23 | DIC |
| ATOM | 1097 | NE2 | GLN | 141 | 20.327 | 52.972 | 84.860 | 1.00 | 18.11 | DIC |
| ATOM | 1098 | C | GLN | 141 | 18.147 | 47.878 | 82.624 | 1.00 | 19.56 | DIC |
| ATOM | 1099 | O | GLN | 141 | 18.071 | 46.822 | 83.258 | 1.00 | 20.21 | DIC |
| ATOM | 1100 | N | LYS | 142 | 17.495 | 48.070 | 81.479 | 1.00 | 18.99 | DIC |
| ATOM | 1101 | CA | LYS | 142 | 16.653 | 47.019 | 80.915 | 1.00 | 18.42 | DIC |
| ATOM | 1102 | CB | LYS | 142 | 15.947 | 47.503 | 79.644 | 1.00 | 20.49 | DIC |
| ATOM | 1103 | CG | LYS | 142 | 14.882 | 48.562 | 79.871 | 1.00 | 23.32 | DIC |
| ATOM | 1104 | CD | LYS | 142 | 14.220 | 48.950 | 78.552 | 1.00 | 24.72 | DIC |
| ATOM | 1105 | CE | LYS | 142 | 13.110 | 49.968 | 78.761 | 1.00 | 26.12 | DIC |
| ATOM | 1106 | NZ | LYS | 142 | 12.037 | 49.424 | 79.644 | 1.00 | 26.72 | DIC |
| ATOM | 1107 | C | LYS | 142 | 17.477 | 45.778 | 80.580 | 1.00 | 18.39 | DIC |
| ATOM | 1108 | O | LYS | 142 | 16.951 | 44.661 | 80.557 | 1.00 | 17.06 | DIC |
| ATOM | 1109 | N | ILE | 143 | 18.764 | 45.957 | 80.294 | 1.00 | 16.69 | DIC |
| ATOM | 1110 | CA | ILE | 143 | 19.569 | 44.785 | 79.985 | 1.00 | 15.20 | DIC |
| ATOM | 1111 | CB | ILE | 143 | 20.389 | 44.964 | 78.680 | 1.00 | 14.48 | DIC |
| ATOM | 1112 | CG2 | ILE | 143 | 19.438 | 45.218 | 77.519 | 1.00 | 15.42 | DIC |
| ATOM | 1113 | CG1 | ILE | 143 | 21.384 | 46.112 | 78.811 | 1.00 | 14.17 | DIC |
| ATOM | 1114 | CD | ILE | 143 | 22.379 | 46.166 | 77.652 | 1.00 | 14.63 | DIC |
| ATOM | 1115 | C | ILE | 143 | 20.471 | 44.362 | 81.143 | 1.00 | 14.14 | DIC |
| ATOM | 1116 | O | ILE | 143 | 21.477 | 43.685 | 80.943 | 1.00 | 14.10 | DIC |
| ATOM | 1117 | N | GLY | 144 | 20.089 | 44.767 | 82.355 | 1.00 | 14.85 | DIC |
| ATOM | 1118 | CA | GLY | 144 | 20.816 | 44.381 | 83.557 | 1.00 | 13.42 | DIC |
| ATOM | 1119 | C | GLY | 144 | 22.048 | 45.154 | 83.984 | 1.00 | 15.34 | DIC |
| ATOM | 1120 | O | GLY | 144 | 22.775 | 44.721 | 84.884 | 1.00 | 14.31 | DIC |
| ATOM | 1121 | N | MSE | 145 | 22.295 | 46.298 | 83.361 | 1.00 | 14.04 | DIC |
| ATOM | 1122 | CA | MSE | 145 | 23.458 | 47.091 | 83.720 | 1.00 | 15.25 | DIC |
| ATOM | 1123 | CB | MSE | 145 | 23.997 | 47.815 | 82.485 | 1.00 | 14.45 | DIC |
| ATOM | 1124 | CG | MSE | 145 | 24.524 | 46.865 | 81.415 | 1.00 | 15.86 | DIC |
| ATOM | 1125 | SE | MSE | 145 | 25.126 | 47.799 | 79.831 | 1.00 | 16.27 | DIC |
| ATOM | 1126 | CE | MSE | 145 | 26.753 | 48.566 | 80.498 | 1.00 | 14.18 | DIC |
| ATOM | 1127 | C | MSE | 145 | 23.115 | 48.099 | 84.809 | 1.00 | 16.09 | DIC |
| ATOM | 1128 | O | MSE | 145 | 21.984 | 48.585 | 84.882 | 1.00 | 14.76 | DIC |
| ATOM | 1129 | N | ALA | 146 | 24.096 | 48.400 | 85.655 | 1.00 | 17.00 | DIC |
| ATOM | 1130 | CA | ALA | 146 | 23.903 | 49.366 | 86.728 | 1.00 | 18.41 | DIC |
| ATOM | 1131 | CB | ALA | 146 | 24.802 | 49.033 | 87.906 | 1.00 | 18.96 | DIC |
| ATOM | 1132 | C | ALA | 146 | 24.238 | 50.752 | 86.192 | 1.00 | 19.79 | DIC |
| ATOM | 1133 | O | ALA | 146 | 24.793 | 50.885 | 85.098 | 1.00 | 18.39 | DIC |
| ATOM | 1134 | N | ASP | 147 | 23.906 | 51.778 | 86.971 | 1.00 | 19.70 | DIC |
| ATOM | 1135 | CA | ASP | 147 | 24.168 | 53.155 | 86.576 | 1.00 | 20.75 | DIC |
| ATOM | 1136 | CB | ASP | 147 | 23.657 | 54.136 | 87.632 | 1.00 | 24.89 | DIC |
| ATOM | 1137 | CG | ASP | 147 | 23.905 | 55.584 | 87.242 | 1.00 | 28.79 | DIC |
| ATOM | 1138 | OD1 | ASP | 147 | 23.171 | 56.108 | 86.380 | 1.00 | 32.14 | DIC |
| ATOM | 1139 | OD2 | ASP | 147 | 24.845 | 56.199 | 87.787 | 1.00 | 33.35 | DIC |
| ATOM | 1140 | C | ASP | 147 | 25.643 | 53.421 | 86.355 | 1.00 | 18.89 | DIC |
| ATOM | 1141 | O | ASP | 147 | 26.007 | 54.205 | 85.472 | 1.00 | 19.38 | DIC |
| ATOM | 1142 | N | GLY | 148 | 26.483 | 52.771 | 87.158 | 1.00 | 16.62 | DIC |
| ATOM | 1143 | CA | GLY | 148 | 27.924 | 52.959 | 87.058 | 1.00 | 15.83 | DIC |
| ATOM | 1144 | C | GLY | 148 | 28.640 | 52.121 | 86.013 | 1.00 | 14.92 | DIC |
| ATOM | 1145 | O | GLY | 148 | 29.852 | 52.238 | 85.842 | 1.00 | 15.77 | DIC |
| ATOM | 1146 | N | GLU | 149 | 27.908 | 51.257 | 85.320 | 1.00 | 14.62 | DIC |
| ATOM | 1147 | CA | GLU | 149 | 28.512 | 50.434 | 84.280 | 1.00 | 13.91 | DIC |
| ATOM | 1148 | CB | GLU | 149 | 27.948 | 49.015 | 84.331 | 1.00 | 13.40 | DIC |
| ATOM | 1149 | CG | GLU | 149 | 28.174 | 48.342 | 85.680 | 1.00 | 16.25 | DIC |
| ATOM | 1150 | CD | GLU | 149 | 27.669 | 46.912 | 85.717 | 1.00 | 17.21 | DIC |
| ATOM | 1151 | OE1 | GLU | 149 | 26.540 | 46.662 | 85.246 | 1.00 | 17.01 | DIC |
| ATOM | 1152 | OE2 | GLU | 149 | 28.403 | 46.041 | 86.228 | 1.00 | 18.86 | DIC |
| ATOM | 1153 | C | GLU | 149 | 28.133 | 51.128 | 82.984 | 1.00 | 13.92 | DIC |
| ATOM | 1154 | O | GLU | 149 | 26.958 | 51.207 | 82.635 | 1.00 | 14.24 | DIC |
| ATOM | 1155 | N | TYR | 150 | 29.131 | 51.641 | 82.280 | 1.00 | 13.00 | DIC |
| ATOM | 1156 | CA | TYR | 150 | 28.880 | 52.386 | 81.056 | 1.00 | 14.46 | DIC |
| ATOM | 1157 | CB | TYR | 150 | 29.968 | 53.449 | 80.904 | 1.00 | 14.10 | DIC |
| ATOM | 1158 | CG | TYR | 150 | 30.069 | 54.308 | 82.151 | 1.00 | 14.23 | DIC |
| ATOM | 1159 | CD1 | TYR | 150 | 31.304 | 54.579 | 82.734 | 1.00 | 14.50 | DIC |
| ATOM | 1160 | CE1 | TYR | 150 | 31.401 | 55.298 | 83.924 | 1.00 | 15.54 | DIC |
| ATOM | 1161 | CD2 | TYR | 150 | 28.920 | 54.787 | 82.789 | 1.00 | 14.65 | DIC |
| ATOM | 1162 | CE2 | TYR | 150 | 29.006 | 55.510 | 83.987 | 1.00 | 15.14 | DIC |
| ATOM | 1163 | CZ | TYR | 150 | 30.256 | 55.756 | 84.545 | 1.00 | 16.39 | DIC |
| ATOM | 1164 | OH | TYR | 150 | 30.373 | 56.440 | 85.728 | 1.00 | 15.21 | DIC |
| ATOM | 1165 | C | TYR | 150 | 28.724 | 51.555 | 79.789 | 1.00 | 13.06 | DIC |
| ATOM | 1166 | O | TYR | 150 | 29.524 | 50.665 | 79.495 | 1.00 | 12.78 | DIC |
| ATOM | 1167 | N | TYR | 151 | 27.663 | 51.858 | 79.049 | 1.00 | 11.90 | DIC |
| ATOM | 1168 | CA | TYR | 151 | 27.351 | 51.143 | 77.819 | 1.00 | 12.53 | DIC |
| ATOM | 1169 | CB | TYR | 151 | 25.931 | 51.509 | 77.374 | 1.00 | 12.20 | DIC |
| ATOM | 1170 | CG | TYR | 151 | 25.352 | 50.644 | 76.274 | 1.00 | 11.21 | DIC |
| ATOM | 1171 | CD1 | TYR | 151 | 25.752 | 50.802 | 74.951 | 1.00 | 9.91 | DIC |
| ATOM | 1172 | CE1 | TYR | 151 | 25.195 | 50.034 | 73.930 | 1.00 | 10.32 | DIC |
| ATOM | 1173 | CD2 | TYR | 151 | 24.373 | 49.687 | 76.557 | 1.00 | 11.48 | DIC |
| ATOM | 1174 | CE2 | TYR | 151 | 23.810 | 48.911 | 75.546 | 1.00 | 11.82 | DIC |
| ATOM | 1175 | CZ | TYR | 151 | 24.225 | 49.090 | 74.230 | 1.00 | 12.60 | DIC |
| ATOM | 1176 | OH | TYR | 151 | 23.668 | 48.330 | 73.218 | 1.00 | 10.81 | DIC |
| ATOM | 1177 | C | TYR | 151 | 28.372 | 51.512 | 76.751 | 1.00 | 11.38 | DIC |
| ATOM | 1178 | O | TYR | 151 | 28.577 | 52.689 | 76.458 | 1.00 | 11.36 | DIC |
| ATOM | 1179 | N | PHE | 152 | 29.041 | 50.513 | 76.190 | 1.00 | 10.71 | DIC |
| ATOM | 1180 | CA | PHE | 152 | 30.030 | 50.812 | 75.168 | 1.00 | 10.99 | DIC |
| ATOM | 1181 | CB | PHE | 152 | 31.337 | 50.034 | 75.416 | 1.00 | 10.34 | DIC |
| ATOM | 1182 | CG | PHE | 152 | 31.193 | 48.533 | 75.372 | 1.00 | 10.77 | DIC |
| ATOM | 1183 | CD1 | PHE | 152 | 30.968 | 47.802 | 76.539 | 1.00 | 9.58 | DIC |
| ATOM | 1184 | CD2 | PHE | 152 | 31.324 | 47.844 | 74.166 | 1.00 | 10.11 | DIC |
| ATOM | 1185 | CE1 | PHE | 152 | 30.879 | 46.399 | 76.507 | 1.00 | 12.23 | DIC |
| ATOM | 1186 | CE2 | PHE | 152 | 31.236 | 46.445 | 74.122 | 1.00 | 11.41 | DIC |
| ATOM | 1187 | CZ | PHE | 152 | 31.015 | 45.720 | 75.291 | 1.00 | 10.68 | DIC |
| ATOM | 1188 | C | PHE | 152 | 29.502 | 50.541 | 73.768 | 1.00 | 11.13 | DIC |
| ATOM | 1189 | O | PHE | 152 | 28.519 | 49.813 | 73.597 | 1.00 | 12.61 | DIC |
| ATOM | 1190 | N | ASN | 153 | 30.131 | 51.161 | 72.772 | 1.00 | 10.37 | DIC |
| ATOM | 1191 | CA | ASN | 153 | 29.732 | 50.962 | 71.385 | 1.00 | 8.75 | DIC |
| ATOM | 1192 | CB | ASN | 153 | 29.969 | 52.231 | 70.553 | 1.00 | 10.03 | DIC |
| ATOM | 1193 | CG | ASN | 153 | 29.579 | 52.052 | 69.092 | 1.00 | 8.59 | DIC |
| ATOM | 1194 | OD1 | ASN | 153 | 30.396 | 51.662 | 68.260 | 1.00 | 9.43 | DIC |
| ATOM | 1195 | ND2 | ASN | 153 | 28.319 | 52.314 | 68.785 | 1.00 | 10.09 | DIC |
| ATOM | 1196 | C | ASN | 153 | 30.560 | 49.802 | 70.854 | 1.00 | 8.80 | DIC |
| ATOM | 1197 | O | ASN | 153 | 31.768 | 49.738 | 71.073 | 1.00 | 9.14 | DIC |
| ATOM | 1198 | N | ALA | 154 | 29.914 | 48.879 | 70.154 | 1.00 | 9.45 | DIC |
| ATOM | 1199 | CA | ALA | 154 | 30.617 | 47.702 | 69.651 | 1.00 | 10.62 | DIC |
| ATOM | 1200 | CB | ALA | 154 | 29.634 | 46.546 | 69.518 | 1.00 | 11.77 | DIC |
| ATOM | 1201 | C | ALA | 154 | 31.368 | 47.895 | 68.339 | 1.00 | 9.97 | DIC |
| ATOM | 1202 | O | ALA | 154 | 31.940 | 46.944 | 67.814 | 1.00 | 10.61 | DIC |
| ATOM | 1203 | N | GLY | 155 | 31.381 | 49.119 | 67.818 | 1.00 | 8.84 | DIC |
| ATOM | 1204 | CA | GLY | 155 | 32.054 | 49.377 | 66.553 | 1.00 | 9.49 | DIC |
| ATOM | 1205 | C | GLY | 155 | 33.575 | 49.396 | 66.545 | 1.00 | 9.26 | DIC |
| ATOM | 1206 | O | GLY | 155 | 34.193 | 49.248 | 65.485 | 1.00 | 9.60 | DIC |
| ATOM | 1207 | N | VAL | 156 | 34.183 | 49.589 | 67.715 | 1.00 | 8.00 | DIC |
| ATOM | 1208 | CA | VAL | 156 | 35.640 | 49.631 | 67.849 | 1.00 | 6.16 | DIC |
| ATOM | 1209 | CB | VAL | 156 | 36.152 | 51.095 | 67.977 | 1.00 | 8.60 | DIC |
| ATOM | 1210 | CG1 | VAL | 156 | 37.667 | 51.111 | 68.200 | 1.00 | 8.18 | DIC |
| ATOM | 1211 | CG2 | VAL | 156 | 35.788 | 51.887 | 66.708 | 1.00 | 4.42 | DIC |
| ATOM | 1212 | C | VAL | 156 | 35.968 | 48.869 | 69.120 | 1.00 | 7.98 | DIC |
| ATOM | 1213 | O | VAL | 156 | 35.711 | 49.351 | 70.225 | 1.00 | 7.64 | DIC |
| ATOM | 1214 | N | LEU | 157 | 36.540 | 47.679 | 68.973 | 1.00 | 7.93 | DIC |
| ATOM | 1215 | CA | LEU | 157 | 36.826 | 46.861 | 70.140 | 1.00 | 8.32 | DIC |
| ATOM | 1216 | CB | LEU | 157 | 35.812 | 45.714 | 70.216 | 1.00 | 8.82 | DIC |
| ATOM | 1217 | CG | LEU | 157 | 34.317 | 46.025 | 70.104 | 1.00 | 6.30 | DIC |
| ATOM | 1218 | CD1 | LEU | 157 | 33.579 | 44.733 | 69.815 | 1.00 | 3.81 | DIC |
| ATOM | 1219 | CD2 | LEU | 157 | 33.804 | 46.675 | 71.373 | 1.00 | 6.09 | DIC |
| ATOM | 1220 | C | LEU | 157 | 38.218 | 46.259 | 70.190 | 1.00 | 10.43 | DIC |
| ATOM | 1221 | O | LEU | 157 | 38.623 | 45.544 | 69.270 | 1.00 | 10.91 | DIC |
| ATOM | 1222 | N | LEU | 158 | 38.948 | 46.546 | 71.265 | 1.00 | 10.32 | DIC |
| ATOM | 1223 | CA | LEU | 158 | 40.270 | 45.967 | 71.444 | 1.00 | 11.68 | DIC |
| ATOM | 1224 | CB | LEU | 158 | 41.172 | 46.887 | 72.266 | 1.00 | 13.62 | DIC |
| ATOM | 1225 | CG | LEU | 158 | 42.617 | 46.400 | 72.369 | 1.00 | 14.82 | DIC |
| ATOM | 1226 | CD1 | LEU | 158 | 43.239 | 46.368 | 70.981 | 1.00 | 14.39 | DIC |
| ATOM | 1227 | CD2 | LEU | 158 | 43.409 | 47.323 | 73.283 | 1.00 | 16.11 | DIC |
| ATOM | 1228 | C | LEU | 158 | 39.929 | 44.705 | 72.227 | 1.00 | 12.27 | DIC |
| ATOM | 1229 | O | LEU | 158 | 39.531 | 44.767 | 73.396 | 1.00 | 13.47 | DIC |
| ATOM | 1230 | N | ILE | 159 | 40.082 | 43.557 | 71.586 | 1.00 | 12.60 | DIC |
| ATOM | 1231 | CA | ILE | 159 | 39.707 | 42.300 | 72.216 | 1.00 | 12.69 | DIC |
| ATOM | 1232 | CB | ILE | 159 | 38.874 | 41.471 | 71.212 | 1.00 | 12.19 | DIC |
| ATOM | 1233 | CG2 | ILE | 159 | 38.528 | 40.093 | 71.792 | 1.00 | 9.38 | DIC |
| ATOM | 1234 | CG1 | ILE | 159 | 37.630 | 42.286 | 70.843 | 1.00 | 9.30 | DIC |
| ATOM | 1235 | CD | ILE | 159 | 36.700 | 41.635 | 69.869 | 1.00 | 13.02 | DIC |
| ATOM | 1236 | C | ILE | 159 | 40.811 | 41.440 | 72.816 | 1.00 | 13.96 | DIC |
| ATOM | 1237 | O | ILE | 159 | 41.850 | 41.185 | 72.200 | 1.00 | 12.79 | DIC |
| ATOM | 1238 | N | ASN | 160 | 40.570 | 41.009 | 74.048 | 1.00 | 14.07 | DIC |
| ATOM | 1239 | CA | ASN | 160 | 41.498 | 40.142 | 74.757 | 1.00 | 13.56 | DIC |
| ATOM | 1240 | CB | ASN | 160 | 41.284 | 40.294 | 76.260 | 1.00 | 14.31 | DIC |
| ATOM | 1241 | CG | ASN | 160 | 42.227 | 39.443 | 77.074 | 1.00 | 13.83 | DIC |
| ATOM | 1242 | OD1 | ASN | 160 | 42.806 | 38.490 | 76.571 | 1.00 | 13.36 | DIC |
| ATOM | 1243 | ND2 | ASN | 160 | 42.374 | 39.778 | 78.347 | 1.00 | 12.66 | DIC |
| ATOM | 1244 | C | ASN | 160 | 41.120 | 38.730 | 74.301 | 1.00 | 13.20 | DIC |
| ATOM | 1245 | O | ASN | 160 | 40.485 | 37.980 | 75.041 | 1.00 | 12.35 | DIC |
| ATOM | 1246 | N | LEU | 161 | 41.497 | 38.380 | 73.073 | 1.00 | 14.48 | DIC |
| ATOM | 1247 | CA | LEU | 161 | 41.173 | 37.071 | 72.506 | 1.00 | 14.41 | DIC |
| ATOM | 1248 | CB | LEU | 161 | 41.772 | 36.927 | 71.103 | 1.00 | 15.27 | DIC |
| ATOM | 1249 | CG | LEU | 161 | 40.849 | 37.142 | 69.902 | 1.00 | 19.60 | DIC |
| ATOM | 1250 | CD1 | LEU | 161 | 41.625 | 36.854 | 68.629 | 1.00 | 17.54 | DIC |
| ATOM | 1251 | CD2 | LEU | 161 | 39.625 | 36.239 | 69.998 | 1.00 | 18.80 | DIC |
| ATOM | 1252 | C | LEU | 161 | 41.601 | 35.873 | 73.345 | 1.00 | 14.82 | DIC |
| ATOM | 1253 | O | LEU | 161 | 40.904 | 34.857 | 73.383 | 1.00 | 12.20 | DIC |
| ATOM | 1254 | N | LYS | 162 | 42.762 | 35.973 | 73.982 | 1.00 | 16.27 | DIC |
| ATOM | 1255 | CA | LYS | 162 | 43.248 | 34.885 | 74.819 | 1.00 | 18.17 | DIC |
| ATOM | 1256 | CB | LYS | 162 | 44.579 | 35.269 | 75.472 | 1.00 | 23.17 | DIC |
| ATOM | 1257 | CG | LYS | 162 | 45.039 | 34.285 | 76.535 | 1.00 | 28.53 | DIC |
| ATOM | 1258 | CD | LYS | 162 | 46.494 | 34.495 | 76.924 | 1.00 | 32.40 | DIC |
| ATOM | 1259 | CE | LYS | 162 | 47.429 | 33.990 | 75.829 | 1.00 | 36.43 | DIC |
| ATOM | 1260 | NZ | LYS | 162 | 48.845 | 33.936 | 76.294 | 1.00 | 38.35 | DIC |
| ATOM | 1261 | C | LYS | 162 | 42.204 | 34.572 | 75.891 | 1.00 | 18.73 | DIC |
| ATOM | 1262 | O | LYS | 162 | 41.927 | 33.406 | 76.178 | 1.00 | 17.49 | DIC |
| ATOM | 1263 | N | LYS | 163 | 41.622 | 35.618 | 76.481 | 1.00 | 16.83 | DIC |
| ATOM | 1264 | CA | LYS | 163 | 40.603 | 35.431 | 77.508 | 1.00 | 15.63 | DIC |
| ATOM | 1265 | CB | LYS | 163 | 40.269 | 36.762 | 78.188 | 1.00 | 14.80 | DIC |
| ATOM | 1266 | CG | LYS | 163 | 39.303 | 36.629 | 79.363 | 1.00 | 16.56 | DIC |
| ATOM | 1267 | CD | LYS | 163 | 39.014 | 37.984 | 79.999 | 1.00 | 17.80 | DIC |
| ATOM | 1268 | CE | LYS | 163 | 38.043 | 37.863 | 81.172 | 1.00 | 19.19 | DIC |
| ATOM | 1269 | NZ | LYS | 163 | 38.560 | 36.924 | 82.210 | 1.00 | 24.46 | DIC |
| ATOM | 1270 | C | LYS | 163 | 39.334 | 34.830 | 76.891 | 1.00 | 15.48 | DIC |
| ATOM | 1271 | O | LYS | 163 | 38.760 | 33.888 | 77.436 | 1.00 | 15.56 | DIC |
| ATOM | 1272 | N | TRP | 164 | 38.896 | 35.376 | 75.758 | 1.00 | 13.99 | DIC |
| ATOM | 1273 | CA | TRP | 164 | 37.703 | 34.857 | 75.088 | 1.00 | 14.31 | DIC |
| ATOM | 1274 | CB | TRP | 164 | 37.453 | 35.598 | 73.773 | 1.00 | 12.76 | DIC |
| ATOM | 1275 | CG | TRP | 164 | 36.885 | 36.979 | 73.909 | 1.00 | 12.61 | DIC |
| ATOM | 1276 | CD2 | TRP | 164 | 35.948 | 37.604 | 73.024 | 1.00 | 11.45 | DIC |
| ATOM | 1277 | CE2 | TRP | 164 | 35.742 | 38.923 | 73.493 | 1.00 | 10.90 | DIC |
| ATOM | 1278 | CE3 | TRP | 164 | 35.265 | 37.179 | 71.876 | 1.00 | 11.56 | DIC |
| ATOM | 1279 | CD1 | TRP | 164 | 37.200 | 37.914 | 74.855 | 1.00 | 12.23 | DIC |
| ATOM | 1280 | NE1 | TRP | 164 | 36.518 | 39.084 | 74.612 | 1.00 | 12.19 | DIC |
| ATOM | 1281 | CZ2 | TRP | 164 | 34.880 | 39.823 | 72.852 | 1.00 | 10.67 | DIC |
| ATOM | 1282 | CZ3 | TRP | 164 | 34.406 | 38.076 | 71.238 | 1.00 | 10.59 | DIC |
| ATOM | 1283 | CH2 | TRP | 164 | 34.224 | 39.382 | 71.729 | 1.00 | 11.25 | DIC |
| ATOM | 1284 | C | TRP | 164 | 37.828 | 33.359 | 74.782 | 1.00 | 14.32 | DIC |
| ATOM | 1285 | O | TRP | 164 | 36.862 | 32.607 | 74.925 | 1.00 | 12.37 | DIC |
| ATOM | 1286 | N | ARG | 165 | 39.009 | 32.927 | 74.349 | 1.00 | 14.03 | DIC |
| ATOM | 1287 | CA | ARG | 165 | 39.191 | 31.519 | 74.008 | 1.00 | 15.65 | DIC |
| ATOM | 1288 | CB | ARG | 165 | 40.515 | 31.307 | 73.260 | 1.00 | 17.22 | DIC |
| ATOM | 1289 | CG | ARG | 165 | 40.498 | 31.881 | 71.833 | 1.00 | 18.70 | DIC |
| ATOM | 1290 | CD | ARG | 165 | 41.599 | 31.285 | 70.945 | 1.00 | 20.78 | DIC |
| ATOM | 1291 | NE | ARG | 165 | 42.933 | 31.672 | 71.394 | 1.00 | 20.86 | DIC |
| ATOM | 1292 | CZ | ARG | 165 | 43.595 | 32.737 | 70.954 | 1.00 | 22.03 | DIC |
| ATOM | 1293 | NH1 | ARG | 165 | 44.802 | 33.012 | 71.433 | 1.00 | 22.53 | DIC |
| ATOM | 1294 | NH2 | ARG | 165 | 43.064 | 33.513 | 70.018 | 1.00 | 23.03 | DIC |
| ATOM | 1295 | C | ARG | 165 | 39.084 | 30.589 | 75.213 | 1.00 | 16.07 | DIC |
| ATOM | 1296 | O | ARG | 165 | 38.959 | 29.377 | 75.061 | 1.00 | 15.48 | DIC |
| ATOM | 1297 | N | ARG | 166 | 39.105 | 31.159 | 76.411 | 1.00 | 17.98 | DIC |
| ATOM | 1298 | CA | ARG | 166 | 38.971 | 30.364 | 77.623 | 1.00 | 18.39 | DIC |
| ATOM | 1299 | CB | ARG | 166 | 39.585 | 31.090 | 78.822 | 1.00 | 21.23 | DIC |
| ATOM | 1300 | CG | ARG | 166 | 41.090 | 31.236 | 78.787 | 1.00 | 23.66 | DIC |
| ATOM | 1301 | CD | ARG | 166 | 41.585 | 31.922 | 80.051 | 1.00 | 24.40 | DIC |
| ATOM | 1302 | NE | ARG | 166 | 43.031 | 32.101 | 80.017 | 1.00 | 28.45 | DIC |
| ATOM | 1303 | CZ | ARG | 166 | 43.644 | 33.276 | 79.929 | 1.00 | 28.36 | DIC |
| ATOM | 1304 | NH1 | ARG | 166 | 42.940 | 34.403 | 79.870 | 1.00 | 28.09 | DIC |
| ATOM | 1305 | NH2 | ARG | 166 | 44.968 | 33.321 | 79.885 | 1.00 | 30.30 | DIC |
| ATOM | 1306 | C | ARG | 166 | 37.489 | 30.141 | 77.906 | 1.00 | 18.07 | DIC |
| ATOM | 1307 | O | ARG | 166 | 37.133 | 29.484 | 78.879 | 1.00 | 16.59 | DIC |
| ATOM | 1308 | N | HIS | 167 | 36.625 | 30.688 | 77.056 | 1.00 | 16.94 | DIC |
| ATOM | 1309 | CA | HIS | 167 | 35.188 | 30.563 | 77.273 | 1.00 | 16.99 | DIC |
| ATOM | 1310 | CB | HIS | 167 | 34.652 | 31.848 | 77.920 | 1.00 | 17.50 | DIC |
| ATOM | 1311 | CG | HIS | 167 | 35.333 | 32.218 | 79.201 | 1.00 | 17.81 | DIC |
| ATOM | 1312 | CD2 | HIS | 167 | 36.422 | 32.989 | 79.441 | 1.00 | 16.88 | DIC |
| ATOM | 1313 | ND1 | HIS | 167 | 34.896 | 31.780 | 80.434 | 1.00 | 19.03 | DIC |
| ATOM | 1314 | CE1 | HIS | 167 | 35.683 | 32.266 | 81.378 | 1.00 | 19.16 | DIC |
| ATOM | 1315 | NE2 | HIS | 167 | 36.617 | 33.004 | 80.802 | 1.00 | 18.25 | DIC |
| ATOM | 1316 | C | HIS | 167 | 34.413 | 30.311 | 75.987 | 1.00 | 17.81 | DIC |
| ATOM | 1317 | O | HIS | 167 | 34.939 | 30.471 | 74.884 | 1.00 | 18.91 | DIC |
| ATOM | 1318 | N | ASP | 168 | 33.154 | 29.916 | 76.141 | 1.00 | 16.56 | DIC |
| ATOM | 1319 | CA | ASP | 168 | 32.287 | 29.682 | 74.997 | 1.00 | 15.52 | DIC |
| ATOM | 1320 | CB | ASP | 168 | 31.461 | 28.406 | 75.187 | 1.00 | 14.57 | DIC |
| ATOM | 1321 | CG | ASP | 168 | 30.727 | 28.001 | 73.921 | 1.00 | 16.35 | DIC |
| ATOM | 1322 | OD1 | ASP | 168 | 30.399 | 26.806 | 73.768 | 1.00 | 16.74 | DIC |
| ATOM | 1323 | OD2 | ASP | 168 | 30.473 | 28.886 | 73.075 | 1.00 | 16.42 | DIC |
| ATOM | 1324 | C | ASP | 168 | 31.377 | 30.900 | 74.878 | 1.00 | 14.56 | DIC |
| ATOM | 1325 | O | ASP | 168 | 30.244 | 30.905 | 75.361 | 1.00 | 15.15 | DIC |
| ATOM | 1326 | N | ILE | 169 | 31.893 | 31.934 | 74.221 | 1.00 | 14.02 | DIC |
| ATOM | 1327 | CA | ILE | 169 | 31.169 | 33.186 | 74.040 | 1.00 | 14.18 | DIC |
| ATOM | 1328 | CB | ILE | 169 | 32.044 | 34.220 | 73.308 | 1.00 | 13.81 | DIC |
| ATOM | 1329 | CG2 | ILE | 169 | 31.311 | 35.554 | 73.224 | 1.00 | 12.37 | DIC |
| ATOM | 1330 | CG1 | ILE | 169 | 33.388 | 34.373 | 74.032 | 1.00 | 14.52 | DIC |
| ATOM | 1331 | CD | ILE | 169 | 33.270 | 34.779 | 75.498 | 1.00 | 15.00 | DIC |
| ATOM | 1332 | C | ILE | 169 | 29.849 | 33.038 | 73.281 | 1.00 | 13.80 | DIC |
| ATOM | 1333 | O | ILE | 169 | 28.873 | 33.729 | 73.584 | 1.00 | 12.98 | DIC |
| ATOM | 1334 | N | PHE | 170 | 29.809 | 32.145 | 72.300 | 1.00 | 14.34 | DIC |
| ATOM | 1335 | CA | PHE | 170 | 28.576 | 31.957 | 71.539 | 1.00 | 14.12 | DIC |
| ATOM | 1336 | CB | PHE | 170 | 28.818 | 31.075 | 70.317 | 1.00 | 15.04 | DIC |
| ATOM | 1337 | CG | PHE | 170 | 27.591 | 30.866 | 69.482 | 1.00 | 16.16 | DIC |
| ATOM | 1338 | CD1 | PHE | 170 | 26.936 | 29.636 | 69.476 | 1.00 | 18.23 | DIC |
| ATOM | 1339 | CD2 | PHE | 170 | 27.067 | 31.909 | 68.732 | 1.00 | 17.64 | DIC |
| ATOM | 1340 | CE1 | PHE | 170 | 25.770 | 29.449 | 68.733 | 1.00 | 19.07 | DIC |
| ATOM | 1341 | CE2 | PHE | 170 | 25.903 | 31.738 | 67.987 | 1.00 | 18.59 | DIC |
| ATOM | 1342 | CZ | PHE | 170 | 25.250 | 30.502 | 67.987 | 1.00 | 19.29 | DIC |
| ATOM | 1343 | C | PHE | 170 | 27.485 | 31.343 | 72.412 | 1.00 | 14.67 | DIC |
| ATOM | 1344 | O | PHE | 170 | 26.321 | 31.738 | 72.342 | 1.00 | 14.40 | DIC |
| ATOM | 1345 | N | LYS | 171 | 27.860 | 30.380 | 73.244 | 1.00 | 14.30 | DIC |
| ATOM | 1346 | CA | LYS | 171 | 26.887 | 29.755 | 74.120 | 1.00 | 15.30 | DIC |
| ATOM | 1347 | CB | LYS | 171 | 27.521 | 28.566 | 74.841 | 1.00 | 17.59 | DIC |
| ATOM | 1348 | CG | LYS | 171 | 26.511 | 27.636 | 75.474 | 1.00 | 22.27 | DIC |
| ATOM | 1349 | CD | LYS | 171 | 27.160 | 26.339 | 75.928 | 1.00 | 24.47 | DIC |
| ATOM | 1350 | CE | LYS | 171 | 27.627 | 25.502 | 74.744 | 1.00 | 27.47 | DIC |
| ATOM | 1351 | NZ | LYS | 171 | 28.299 | 24.238 | 75.192 | 1.00 | 31.51 | DIC |
| ATOM | 1352 | C | LYS | 171 | 26.396 | 30.802 | 75.121 | 1.00 | 14.40 | DIC |
| ATOM | 1353 | O | LYS | 171 | 25.197 | 30.933 | 75.355 | 1.00 | 15.04 | DIC |
| ATOM | 1354 | N | MSE | 172 | 27.327 | 31.553 | 75.702 | 1.00 | 12.53 | DIC |
| ATOM | 1355 | CA | MSE | 172 | 26.969 | 32.599 | 76.656 | 1.00 | 12.42 | DIC |
| ATOM | 1356 | CB | MSE | 172 | 28.232 | 33.280 | 77.203 | 1.00 | 12.59 | DIC |
| ATOM | 1357 | CG | MSE | 172 | 29.087 | 32.407 | 78.120 | 1.00 | 14.48 | DIC |
| ATOM | 1358 | SE | MSE | 172 | 30.875 | 33.131 | 78.357 | 1.00 | 19.27 | DIC |
| ATOM | 1359 | CE | MSE | 172 | 30.437 | 34.845 | 79.143 | 1.00 | 17.17 | DIC |
| ATOM | 1360 | C | MSE | 172 | 26.082 | 33.640 | 75.963 | 1.00 | 12.96 | DIC |
| ATOM | 1361 | O | MSE | 172 | 25.175 | 34.199 | 76.583 | 1.00 | 12.64 | DIC |
| ATOM | 1362 | N | SER | 173 | 26.347 | 33.892 | 74.679 | 1.00 | 11.38 | DIC |
| ATOM | 1363 | CA | SER | 173 | 25.566 | 34.863 | 73.914 | 1.00 | 11.90 | DIC |
| ATOM | 1364 | CB | SER | 173 | 26.205 | 35.118 | 72.542 | 1.00 | 12.42 | DIC |
| ATOM | 1365 | OG | SER | 173 | 27.411 | 35.867 | 72.657 | 1.00 | 11.32 | DIC |
| ATOM | 1366 | C | SER | 173 | 24.141 | 34.356 | 73.724 | 1.00 | 12.64 | DIC |
| ATOM | 1367 | O | SER | 173 | 23.172 | 35.079 | 73.988 | 1.00 | 9.99 | DIC |
| ATOM | 1368 | N | SER | 174 | 24.026 | 33.107 | 73.275 | 1.00 | 12.51 | DIC |
| ATOM | 1369 | CA | SER | 174 | 22.730 | 32.489 | 73.040 | 1.00 | 13.32 | DIC |
| ATOM | 1370 | CB | SER | 174 | 22.921 | 31.065 | 72.510 | 1.00 | 13.27 | DIC |
| ATOM | 1371 | OG | SER | 174 | 23.581 | 31.100 | 71.259 | 1.00 | 14.11 | DIC |
| ATOM | 1372 | C | SER | 174 | 21.861 | 32.472 | 74.292 | 1.00 | 13.57 | DIC |
| ATOM | 1373 | O | SER | 174 | 20.647 | 32.711 | 74.221 | 1.00 | 13.23 | DIC |
| ATOM | 1374 | N | GLU | 175 | 22.480 | 32.187 | 75.434 | 1.00 | 13.02 | DIC |
| ATOM | 1375 | CA | GLU | 175 | 21.758 | 32.151 | 76.698 | 1.00 | 14.86 | DIC |
| ATOM | 1376 | CB | GLU | 175 | 22.638 | 31.542 | 77.797 | 1.00 | 17.57 | DIC |
| ATOM | 1377 | CG | GLU | 175 | 23.168 | 30.152 | 77.451 | 1.00 | 22.53 | DIC |
| ATOM | 1378 | CD | GLU | 175 | 23.838 | 29.457 | 78.626 | 1.00 | 25.32 | DIC |
| ATOM | 1379 | OE1 | GLU | 175 | 24.535 | 30.136 | 79.413 | 1.00 | 26.06 | DIC |
| ATOM | 1380 | OE2 | GLU | 175 | 23.673 | 28.222 | 78.753 | 1.00 | 28.49 | DIC |
| ATOM | 1381 | C | GLU | 175 | 21.311 | 33.557 | 77.109 | 1.00 | 14.41 | DIC |
| ATOM | 1382 | O | GLU | 175 | 20.216 | 33.737 | 77.648 | 1.00 | 15.15 | DIC |
| ATOM | 1383 | N | TRP | 176 | 22.158 | 34.550 | 76.851 | 1.00 | 14.00 | DIC |
| ATOM | 1384 | CA | TRP | 176 | 21.832 | 35.932 | 77.198 | 1.00 | 13.86 | DIC |
| ATOM | 1385 | CB | TRP | 176 | 23.041 | 36.841 | 76.957 | 1.00 | 14.76 | DIC |
| ATOM | 1386 | CG | TRP | 176 | 22.907 | 38.186 | 77.599 | 1.00 | 14.73 | DIC |
| ATOM | 1387 | CD2 | TRP | 176 | 22.383 | 39.375 | 76.999 | 1.00 | 15.10 | DIC |
| ATOM | 1388 | CE2 | TRP | 176 | 22.400 | 40.383 | 77.987 | 1.00 | 15.08 | DIC |
| ATOM | 1389 | CE3 | TRP | 176 | 21.896 | 39.685 | 75.725 | 1.00 | 14.14 | DIC |
| ATOM | 1390 | CD1 | TRP | 176 | 23.213 | 38.511 | 78.884 | 1.00 | 15.31 | DIC |
| ATOM | 1391 | NE1 | TRP | 176 | 22.912 | 39.829 | 79.128 | 1.00 | 14.86 | DIC |
| ATOM | 1392 | CZ2 | TRP | 176 | 21.949 | 41.684 | 77.738 | 1.00 | 15.02 | DIC |
| ATOM | 1393 | CZ3 | TRP | 176 | 21.446 | 40.979 | 75.479 | 1.00 | 13.42 | DIC |
| ATOM | 1394 | CH2 | TRP | 176 | 21.476 | 41.960 | 76.480 | 1.00 | 14.16 | DIC |
| ATOM | 1395 | C | TRP | 176 | 20.653 | 36.396 | 76.342 | 1.00 | 14.32 | DIC |
| ATOM | 1396 | O | TRP | 176 | 19.709 | 37.024 | 76.842 | 1.00 | 14.65 | DIC |
| ATOM | 1397 | N | VAL | 177 | 20.707 | 36.082 | 75.050 | 1.00 | 13.88 | DIC |
| ATOM | 1398 | CA | VAL | 177 | 19.633 | 36.463 | 74.144 | 1.00 | 13.78 | DIC |
| ATOM | 1399 | CB | VAL | 177 | 19.917 | 36.021 | 72.692 | 1.00 | 13.94 | DIC |
| ATOM | 1400 | CG1 | VAL | 177 | 18.673 | 36.235 | 71.837 | 1.00 | 14.54 | DIC |
| ATOM | 1401 | CG2 | VAL | 177 | 21.072 | 36.828 | 72.112 | 1.00 | 11.49 | DIC |
| ATOM | 1402 | C | VAL | 177 | 18.320 | 35.839 | 74.601 | 1.00 | 15.27 | DIC |
| ATOM | 1403 | O | VAL | 177 | 17.269 | 36.476 | 74.560 | 1.00 | 13.41 | DIC |
| ATOM | 1404 | N | GLU | 178 | 18.373 | 34.591 | 75.043 | 1.00 | 17.23 | DIC |
| ATOM | 1405 | CA | GLU | 178 | 17.157 | 33.933 | 75.491 | 1.00 | 20.02 | DIC |
| ATOM | 1406 | CB | GLU | 178 | 17.469 | 32.505 | 75.952 | 1.00 | 22.23 | DIC |
| ATOM | 1407 | CG | GLU | 178 | 16.236 | 31.648 | 76.208 | 1.00 | 27.72 | DIC |
| ATOM | 1408 | CD | GLU | 178 | 15.214 | 31.727 | 75.078 | 1.00 | 29.81 | DIC |
| ATOM | 1409 | OE1 | GLU | 178 | 15.590 | 31.535 | 73.898 | 1.00 | 31.17 | DIC |
| ATOM | 1410 | OE2 | GLU | 178 | 14.027 | 31.979 | 75.376 | 1.00 | 32.50 | DIC |
| ATOM | 1411 | C | GLU | 178 | 16.549 | 34.735 | 76.636 | 1.00 | 20.30 | DIC |
| ATOM | 1412 | O | GLU | 178 | 15.328 | 34.848 | 76.758 | 1.00 | 20.62 | DIC |
| ATOM | 1413 | N | GLN | 179 | 17.416 | 35.320 | 77.453 | 1.00 | 19.19 | DIC |
| ATOM | 1414 | CA | GLN | 179 | 16.988 | 36.093 | 78.609 | 1.00 | 21.07 | DIC |
| ATOM | 1415 | CB | GLN | 179 | 18.136 | 36.120 | 79.625 | 1.00 | 23.36 | DIC |
| ATOM | 1416 | CG | GLN | 179 | 17.829 | 36.793 | 80.943 | 1.00 | 29.17 | DIC |
| ATOM | 1417 | CD | GLN | 179 | 19.050 | 36.865 | 81.852 | 1.00 | 33.38 | DIC |
| ATOM | 1418 | OE1 | GLN | 179 | 18.995 | 37.432 | 82.947 | 1.00 | 35.19 | DIC |
| ATOM | 1419 | NE2 | GLN | 179 | 20.163 | 36.290 | 81.396 | 1.00 | 34.26 | DIC |
| ATOM | 1420 | C | GLN | 179 | 16.516 | 37.524 | 78.313 | 1.00 | 19.81 | DIC |
| ATOM | 1421 | O | GLN | 179 | 15.593 | 38.018 | 78.956 | 1.00 | 18.51 | DIC |
| ATOM | 1422 | N | TYR | 180 | 17.125 | 38.183 | 77.333 | 1.00 | 18.69 | DIC |
| ATOM | 1423 | CA | TYR | 180 | 16.758 | 39.561 | 77.038 | 1.00 | 17.26 | DIC |
| ATOM | 1424 | CB | TYR | 180 | 17.962 | 40.469 | 77.277 | 1.00 | 17.36 | DIC |
| ATOM | 1425 | CG | TYR | 180 | 18.421 | 40.529 | 78.710 | 1.00 | 18.17 | DIC |
| ATOM | 1426 | CD1 | TYR | 180 | 19.325 | 39.598 | 79.214 | 1.00 | 18.19 | DIC |
| ATOM | 1427 | CE1 | TYR | 180 | 19.773 | 39.675 | 80.528 | 1.00 | 19.56 | DIC |
| ATOM | 1428 | CD2 | TYR | 180 | 17.966 | 41.535 | 79.560 | 1.00 | 19.48 | DIC |
| ATOM | 1429 | CE2 | TYR | 180 | 18.404 | 41.618 | 80.873 | 1.00 | 19.98 | DIC |
| ATOM | 1430 | CZ | TYR | 180 | 19.310 | 40.685 | 81.351 | 1.00 | 19.10 | DIC |
| ATOM | 1431 | OH | TYR | 180 | 19.760 | 40.772 | 82.650 | 1.00 | 21.76 | DIC |
| ATOM | 1432 | C | TYR | 180 | 16.206 | 39.861 | 75.651 | 1.00 | 17.56 | DIC |
| ATOM | 1433 | O | TYR | 180 | 16.068 | 41.030 | 75.288 | 1.00 | 16.75 | DIC |
| ATOM | 1434 | N | LYS | 181 | 15.886 | 38.826 | 74.884 | 1.00 | 17.25 | DIC |
| ATOM | 1435 | CA | LYS | 181 | 15.382 | 39.004 | 73.523 | 1.00 | 19.55 | DIC |
| ATOM | 1436 | CB | LYS | 181 | 15.086 | 37.636 | 72.898 | 1.00 | 22.09 | DIC |
| ATOM | 1437 | CG | LYS | 181 | 13.950 | 36.875 | 73.566 | 1.00 | 24.42 | DIC |
| ATOM | 1438 | CD | LYS | 181 | 13.778 | 35.491 | 72.946 | 1.00 | 28.02 | DIC |
| ATOM | 1439 | CE | LYS | 181 | 12.475 | 34.847 | 73.395 | 1.00 | 28.32 | DIC |
| ATOM | 1440 | NZ | LYS | 181 | 12.358 | 34.833 | 74.878 | 1.00 | 31.53 | DIC |
| ATOM | 1441 | C | LYS | 181 | 14.150 | 39.902 | 73.367 | 1.00 | 20.33 | DIC |
| ATOM | 1442 | O | LYS | 181 | 13.940 | 40.489 | 72.304 | 1.00 | 19.96 | DIC |
| ATOM | 1443 | N | ASP | 182 | 13.336 | 40.012 | 74.410 | 1.00 | 19.39 | DIC |
| ATOM | 1444 | CA | ASP | 182 | 12.132 | 40.830 | 74.320 | 1.00 | 21.97 | DIC |
| ATOM | 1445 | CB | ASP | 182 | 11.053 | 40.288 | 75.258 | 1.00 | 23.67 | DIC |
| ATOM | 1446 | CG | ASP | 182 | 10.476 | 38.965 | 74.785 | 1.00 | 26.84 | DIC |
| ATOM | 1447 | OD1 | ASP | 182 | 9.781 | 38.308 | 75.587 | 1.00 | 29.25 | DIC |
| ATOM | 1448 | OD2 | ASP | 182 | 10.703 | 38.585 | 73.614 | 1.00 | 27.07 | DIC |
| ATOM | 1449 | C | ASP | 182 | 12.368 | 42.298 | 74.634 | 1.00 | 21.11 | DIC |
| ATOM | 1450 | O | ASP | 182 | 11.472 | 43.121 | 74.460 | 1.00 | 21.52 | DIC |
| ATOM | 1451 | N | VAL | 183 | 13.568 | 42.632 | 75.096 | 1.00 | 19.54 | DIC |
| ATOM | 1452 | CA | VAL | 183 | 13.861 | 44.015 | 75.435 | 1.00 | 18.64 | DIC |
| ATOM | 1453 | CB | VAL | 183 | 14.093 | 44.160 | 76.968 | 1.00 | 20.70 | DIC |
| ATOM | 1454 | CG1 | VAL | 183 | 15.332 | 43.388 | 77.396 | 1.00 | 19.44 | DIC |
| ATOM | 1455 | CG2 | VAL | 183 | 14.214 | 45.617 | 77.341 | 1.00 | 24.74 | DIC |
| ATOM | 1456 | C | VAL | 183 | 15.050 | 44.606 | 74.682 | 1.00 | 18.43 | DIC |
| ATOM | 1457 | O | VAL | 183 | 15.104 | 45.820 | 74.462 | 1.00 | 18.40 | DIC |
| ATOM | 1458 | N | MSE | 184 | 15.998 | 43.766 | 74.276 | 1.00 | 16.59 | DIC |
| ATOM | 1459 | CA | MSE | 184 | 17.173 | 44.276 | 73.573 | 1.00 | 15.96 | DIC |
| ATOM | 1460 | CB | MSE | 184 | 18.173 | 43.135 | 73.278 | 1.00 | 17.24 | DIC |
| ATOM | 1461 | CG | MSE | 184 | 17.620 | 41.917 | 72.564 | 1.00 | 18.77 | DIC |
| ATOM | 1462 | SE | MSE | 184 | 18.898 | 40.440 | 72.634 | 1.00 | 17.57 | DIC |
| ATOM | 1463 | CE | MSE | 184 | 20.063 | 40.984 | 71.194 | 1.00 | 13.79 | DIC |
| ATOM | 1464 | C | MSE | 184 | 16.804 | 45.050 | 72.305 | 1.00 | 15.73 | DIC |
| ATOM | 1465 | O | MSE | 184 | 15.940 | 44.637 | 71.525 | 1.00 | 13.50 | DIC |
| ATOM | 1466 | N | GLN | 185 | 17.460 | 46.195 | 72.125 | 1.00 | 13.82 | DIC |
| ATOM | 1467 | CA | GLN | 185 | 17.207 | 47.074 | 70.989 | 1.00 | 13.45 | DIC |
| ATOM | 1468 | CB | GLN | 185 | 16.781 | 48.460 | 71.496 | 1.00 | 15.92 | DIC |
| ATOM | 1469 | CG | GLN | 185 | 15.485 | 48.468 | 72.306 | 1.00 | 15.22 | DIC |
| ATOM | 1470 | CD | GLN | 185 | 15.126 | 49.847 | 72.864 | 1.00 | 18.20 | DIC |
| ATOM | 1471 | OE1 | GLN | 185 | 15.648 | 50.875 | 72.417 | 1.00 | 16.73 | DIC |
| ATOM | 1472 | NE2 | GLN | 185 | 14.215 | 49.870 | 73.836 | 1.00 | 15.67 | DIC |
| ATOM | 1473 | C | GLN | 185 | 18.426 | 47.231 | 70.084 | 1.00 | 13.75 | DIC |
| ATOM | 1474 | O | GLN | 185 | 18.301 | 47.694 | 68.945 | 1.00 | 12.35 | DIC |
| ATOM | 1475 | N | TYR | 186 | 19.602 | 46.849 | 70.580 | 1.00 | 10.80 | DIC |
| ATOM | 1476 | CA | TYR | 186 | 20.809 | 47.014 | 69.785 | 1.00 | 12.98 | DIC |
| ATOM | 1477 | CB | TYR | 186 | 21.742 | 48.010 | 70.479 | 1.00 | 14.55 | DIC |
| ATOM | 1478 | CG | TYR | 186 | 21.067 | 49.351 | 70.690 | 1.00 | 15.13 | DIC |
| ATOM | 1479 | CD1 | TYR | 186 | 20.569 | 49.720 | 71.941 | 1.00 | 13.69 | DIC |
| ATOM | 1480 | CE1 | TYR | 186 | 19.868 | 50.918 | 72.116 | 1.00 | 14.58 | DIC |
| ATOM | 1481 | CD2 | TYR | 186 | 20.854 | 50.218 | 69.616 | 1.00 | 17.25 | DIC |
| ATOM | 1482 | CE2 | TYR | 186 | 20.152 | 51.414 | 69.778 | 1.00 | 17.83 | DIC |
| ATOM | 1483 | CZ | TYR | 186 | 19.662 | 51.756 | 71.030 | 1.00 | 16.39 | DIC |
| ATOM | 1484 | OH | TYR | 186 | 18.958 | 52.928 | 71.182 | 1.00 | 17.08 | DIC |
| ATOM | 1485 | C | TYR | 186 | 21.564 | 45.764 | 69.345 | 1.00 | 11.95 | DIC |
| ATOM | 1486 | O | TYR | 186 | 22.758 | 45.813 | 69.056 | 1.00 | 10.67 | DIC |
| ATOM | 1487 | N | GLN | 187 | 20.853 | 44.644 | 69.313 | 1.00 | 10.94 | DIC |
| ATOM | 1488 | CA | GLN | 187 | 21.400 | 43.393 | 68.813 | 1.00 | 10.45 | DIC |
| ATOM | 1489 | CB | GLN | 187 | 21.435 | 43.521 | 67.281 | 1.00 | 11.05 | DIC |
| ATOM | 1490 | CG | GLN | 187 | 20.042 | 43.956 | 66.773 | 1.00 | 12.55 | DIC |
| ATOM | 1491 | CD | GLN | 187 | 19.927 | 44.215 | 65.279 | 1.00 | 13.86 | DIC |
| ATOM | 1492 | OE1 | GLN | 187 | 18.821 | 44.351 | 64.766 | 1.00 | 16.40 | DIC |
| ATOM | 1493 | NE2 | GLN | 187 | 21.048 | 44.293 | 64.582 | 1.00 | 12.57 | DIC |
| ATOM | 1494 | C | GLN | 187 | 22.735 | 42.929 | 69.407 | 1.00 | 9.77 | DIC |
| ATOM | 1495 | O | GLN | 187 | 22.850 | 42.793 | 70.627 | 1.00 | 9.45 | DIC |
| ATOM | 1496 | N | ASP | 188 | 23.741 | 42.683 | 68.568 | 1.00 | 8.20 | DIC |
| ATOM | 1497 | CA | ASP | 188 | 25.030 | 42.205 | 69.071 | 1.00 | 8.35 | DIC |
| ATOM | 1498 | CB | ASP | 188 | 25.999 | 41.978 | 67.916 | 1.00 | 9.13 | DIC |
| ATOM | 1499 | CG | ASP | 188 | 26.220 | 43.224 | 67.111 | 1.00 | 12.26 | DIC |
| ATOM | 1500 | OD1 | ASP | 188 | 25.312 | 43.586 | 66.337 | 1.00 | 13.14 | DIC |
| ATOM | 1501 | OD2 | ASP | 188 | 27.286 | 43.846 | 67.269 | 1.00 | 10.65 | DIC |
| ATOM | 1502 | C | ASP | 188 | 25.674 | 43.146 | 70.089 | 1.00 | 9.52 | DIC |
| ATOM | 1503 | O | ASP | 188 | 26.453 | 42.720 | 70.950 | 1.00 | 8.45 | DIC |
| ATOM | 1504 | N | GLN | 189 | 25.354 | 44.428 | 69.992 | 1.00 | 9.27 | DIC |
| ATOM | 1505 | CA | GLN | 189 | 25.910 | 45.409 | 70.915 | 1.00 | 9.53 | DIC |
| ATOM | 1506 | CB | GLN | 189 | 25.548 | 46.816 | 70.433 | 1.00 | 11.21 | DIC |
| ATOM | 1507 | CG | GLN | 189 | 26.252 | 47.950 | 71.140 | 1.00 | 13.73 | DIC |
| ATOM | 1508 | CD | GLN | 189 | 26.138 | 49.248 | 70.346 | 1.00 | 15.81 | DIC |
| ATOM | 1509 | OE1 | GLN | 189 | 27.060 | 49.627 | 69.622 | 1.00 | 13.26 | DIC |
| ATOM | 1510 | NE2 | GLN | 189 | 24.989 | 49.919 | 70.461 | 1.00 | 13.80 | DIC |
| ATOM | 1511 | C | GLN | 189 | 25.386 | 45.156 | 72.333 | 1.00 | 9.28 | DIC |
| ATOM | 1512 | O | GLN | 189 | 26.136 | 45.251 | 73.300 | 1.00 | 7.78 | DIC |
| ATOM | 1513 | N | ASP | 190 | 24.100 | 44.828 | 72.457 | 1.00 | 9.50 | DIC |
| ATOM | 1514 | CA | ASP | 190 | 23.521 | 44.542 | 73.771 | 1.00 | 10.89 | DIC |
| ATOM | 1515 | CB | ASP | 190 | 22.021 | 44.259 | 73.665 | 1.00 | 9.92 | DIC |
| ATOM | 1516 | CG | ASP | 190 | 21.208 | 45.499 | 73.357 | 1.00 | 11.74 | DIC |
| ATOM | 1517 | OD1 | ASP | 190 | 20.197 | 45.360 | 72.639 | 1.00 | 10.56 | DIC |
| ATOM | 1518 | OD2 | ASP | 190 | 21.570 | 46.600 | 73.841 | 1.00 | 9.10 | DIC |
| ATOM | 1519 | C | ASP | 190 | 24.186 | 43.310 | 74.362 | 1.00 | 9.43 | DIC |
| ATOM | 1520 | O | ASP | 190 | 24.483 | 43.263 | 75.553 | 1.00 | 10.61 | DIC |
| ATOM | 1521 | N | ILE | 191 | 24.389 | 42.302 | 73.524 | 1.00 | 8.72 | DIC |
| ATOM | 1522 | CA | ILE | 191 | 24.998 | 41.061 | 73.970 | 1.00 | 8.21 | DIC |
| ATOM | 1523 | CB | ILE | 191 | 25.125 | 40.056 | 72.811 | 1.00 | 8.37 | DIC |
| ATOM | 1524 | CG2 | ILE | 191 | 25.797 | 38.766 | 73.316 | 1.00 | 6.32 | DIC |
| ATOM | 1525 | CG1 | ILE | 191 | 23.737 | 39.736 | 72.252 | 1.00 | 6.91 | DIC |
| ATOM | 1526 | CD | ILE | 191 | 23.765 | 38.864 | 71.004 | 1.00 | 6.73 | DIC |
| ATOM | 1527 | C | ILE | 191 | 26.368 | 41.277 | 74.590 | 1.00 | 9.22 | DIC |
| ATOM | 1528 | O | ILE | 191 | 26.640 | 40.794 | 75.695 | 1.00 | 9.62 | DIC |
| ATOM | 1529 | N | LEU | 192 | 27.229 | 42.011 | 73.889 | 1.00 | 9.09 | DIC |
| ATOM | 1530 | CA | LEU | 192 | 28.574 | 42.276 | 74.391 | 1.00 | 9.40 | DIC |
| ATOM | 1531 | CB | LEU | 192 | 29.376 | 43.060 | 73.343 | 1.00 | 9.83 | DIC |
| ATOM | 1532 | CG | LEU | 192 | 29.588 | 42.326 | 72.011 | 1.00 | 11.31 | DIC |
| ATOM | 1533 | CD1 | LEU | 192 | 29.942 | 43.307 | 70.901 | 1.00 | 12.97 | DIC |
| ATOM | 1534 | CD2 | LEU | 192 | 30.678 | 41.286 | 72.188 | 1.00 | 12.52 | DIC |
| ATOM | 1535 | C | LEU | 192 | 28.529 | 43.047 | 75.715 | 1.00 | 11.04 | DIC |
| ATOM | 1536 | O | LEU | 192 | 29.272 | 42.734 | 76.652 | 1.00 | 9.18 | DIC |
| ATOM | 1537 | N | ASN | 193 | 27.662 | 44.053 | 75.797 | 1.00 | 10.74 | DIC |
| ATOM | 1538 | CA | ASN | 193 | 27.547 | 44.847 | 77.024 | 1.00 | 12.24 | DIC |
| ATOM | 1539 | CB | ASN | 193 | 26.619 | 46.054 | 76.808 | 1.00 | 10.61 | DIC |
| ATOM | 1540 | CG | ASN | 193 | 27.362 | 47.284 | 76.280 | 1.00 | 13.95 | DIC |
| ATOM | 1541 | OD1 | ASN | 193 | 28.031 | 47.991 | 77.039 | 1.00 | 12.61 | DIC |
| ATOM | 1542 | ND2 | ASN | 193 | 27.251 | 47.536 | 74.974 | 1.00 | 10.56 | DIC |
| ATOM | 1543 | C | ASN | 193 | 27.010 | 43.986 | 78.162 | 1.00 | 12.42 | DIC |
| ATOM | 1544 | O | ASN | 193 | 27.515 | 44.039 | 79.284 | 1.00 | 14.38 | DIC |
| ATOM | 1545 | N | GLY | 194 | 25.981 | 43.198 | 77.872 | 1.00 | 12.71 | DIC |
| ATOM | 1546 | CA | GLY | 194 | 25.407 | 42.348 | 78.898 | 1.00 | 13.05 | DIC |
| ATOM | 1547 | C | GLY | 194 | 26.403 | 41.342 | 79.443 | 1.00 | 13.73 | DIC |
| ATOM | 1548 | O | GLY | 194 | 26.541 | 41.172 | 80.656 | 1.00 | 12.77 | DIC |
| ATOM | 1549 | N | LEU | 195 | 27.122 | 40.679 | 78.547 | 1.00 | 12.99 | DIC |
| ATOM | 1550 | CA | LEU | 195 | 28.086 | 39.674 | 78.969 | 1.00 | 13.81 | DIC |
| ATOM | 1551 | CB | LEU | 195 | 28.533 | 38.826 | 77.769 | 1.00 | 11.07 | DIC |
| ATOM | 1552 | CG | LEU | 195 | 27.562 | 37.911 | 77.016 | 1.00 | 11.26 | DIC |
| ATOM | 1553 | CD1 | LEU | 195 | 28.303 | 37.265 | 75.841 | 1.00 | 8.90 | DIC |
| ATOM | 1554 | CD2 | LEU | 195 | 27.024 | 36.820 | 77.938 | 1.00 | 11.82 | DIC |
| ATOM | 1555 | C | LEU | 195 | 29.339 | 40.199 | 79.675 | 1.00 | 13.59 | DIC |
| ATOM | 1556 | O | LEU | 195 | 29.822 | 39.580 | 80.620 | 1.00 | 12.85 | DIC |
| ATOM | 1557 | N | PHE | 196 | 29.860 | 41.341 | 79.235 | 1.00 | 12.78 | DIC |
| ATOM | 1558 | CA | PHE | 196 | 31.110 | 41.842 | 79.797 | 1.00 | 12.08 | DIC |
| ATOM | 1559 | CB | PHE | 196 | 32.064 | 42.204 | 78.652 | 1.00 | 13.05 | DIC |
| ATOM | 1560 | CG | PHE | 196 | 32.214 | 41.119 | 77.624 | 1.00 | 13.16 | DIC |
| ATOM | 1561 | CD1 | PHE | 196 | 32.244 | 39.780 | 78.004 | 1.00 | 10.72 | DIC |
| ATOM | 1562 | CD2 | PHE | 196 | 32.359 | 41.436 | 76.275 | 1.00 | 13.59 | DIC |
| ATOM | 1563 | CE1 | PHE | 196 | 32.419 | 38.766 | 77.058 | 1.00 | 13.82 | DIC |
| ATOM | 1564 | CE2 | PHE | 196 | 32.534 | 40.430 | 75.318 | 1.00 | 12.70 | DIC |
| ATOM | 1565 | CZ | PHE | 196 | 32.565 | 39.095 | 75.709 | 1.00 | 11.81 | DIC |
| ATOM | 1566 | C | PHE | 196 | 31.071 | 42.993 | 80.786 | 1.00 | 13.04 | DIC |
| ATOM | 1567 | O | PHE | 196 | 32.125 | 43.434 | 81.257 | 1.00 | 11.71 | DIC |
| ATOM | 1568 | N | LYS | 197 | 29.875 | 43.480 | 81.095 | 1.00 | 11.55 | DIC |
| ATOM | 1569 | CA | LYS | 197 | 29.731 | 44.577 | 82.032 | 1.00 | 13.00 | DIC |
| ATOM | 1570 | CB | LYS | 197 | 28.250 | 44.784 | 82.381 | 1.00 | 12.60 | DIC |
| ATOM | 1571 | CG | LYS | 197 | 27.540 | 43.582 | 83.016 | 1.00 | 13.14 | DIC |
| ATOM | 1572 | CD | LYS | 197 | 26.063 | 43.899 | 83.223 | 1.00 | 11.03 | DIC |
| ATOM | 1573 | CE | LYS | 197 | 25.316 | 42.798 | 83.972 | 1.00 | 14.35 | DIC |
| ATOM | 1574 | NZ | LYS | 197 | 25.265 | 41.519 | 83.214 | 1.00 | 14.09 | DIC |
| ATOM | 1575 | C | LYS | 197 | 30.545 | 44.286 | 83.287 | 1.00 | 14.20 | DIC |
| ATOM | 1576 | O | LYS | 197 | 30.494 | 43.182 | 83.826 | 1.00 | 14.43 | DIC |
| ATOM | 1577 | N | GLY | 198 | 31.319 | 45.272 | 83.731 | 1.00 | 15.83 | DIC |
| ATOM | 1578 | CA | GLY | 198 | 32.146 | 45.094 | 84.914 | 1.00 | 16.30 | DIC |
| ATOM | 1579 | C | GLY | 198 | 33.546 | 44.622 | 84.566 | 1.00 | 16.73 | DIC |
| ATOM | 1580 | O | GLY | 198 | 34.457 | 44.678 | 85.395 | 1.00 | 15.89 | DIC |
| ATOM | 1581 | N | GLY | 199 | 33.718 | 44.158 | 83.330 | 1.00 | 15.83 | DIC |
| ATOM | 1582 | CA | GLY | 199 | 35.014 | 43.676 | 82.886 | 1.00 | 15.74 | DIC |
| ATOM | 1583 | C | GLY | 199 | 35.395 | 44.315 | 81.563 | 1.00 | 17.97 | DIC |
| ATOM | 1584 | O | GLY | 199 | 35.854 | 43.644 | 80.641 | 1.00 | 16.51 | DIC |
| ATOM | 1585 | N | VAL | 200 | 35.191 | 45.625 | 81.484 | 1.00 | 16.42 | DIC |
| ATOM | 1586 | CA | VAL | 200 | 35.498 | 46.397 | 80.292 | 1.00 | 17.21 | DIC |
| ATOM | 1587 | CB | VAL | 200 | 34.219 | 47.034 | 79.696 | 1.00 | 16.55 | DIC |
| ATOM | 1588 | CG1 | VAL | 200 | 34.586 | 47.977 | 78.547 | 1.00 | 19.60 | DIC |
| ATOM | 1589 | CG2 | VAL | 200 | 33.275 | 45.951 | 79.204 | 1.00 | 17.01 | DIC |
| ATOM | 1590 | C | VAL | 200 | 36.471 | 47.533 | 80.596 | 1.00 | 16.86 | DIC |
| ATOM | 1591 | O | VAL | 200 | 36.337 | 48.228 | 81.601 | 1.00 | 16.58 | DIC |
| ATOM | 1592 | N | CYS | 201 | 37.454 | 47.708 | 79.720 | 1.00 | 16.59 | DIC |
| ATOM | 1593 | CA | CYS | 201 | 38.412 | 48.793 | 79.853 | 1.00 | 17.05 | DIC |
| ATOM | 1594 | CB | CYS | 201 | 39.802 | 48.345 | 79.391 | 1.00 | 19.85 | DIC |
| ATOM | 1595 | SG | CYS | 201 | 41.031 | 49.677 | 79.319 | 1.00 | 29.46 | DIC |
| ATOM | 1596 | C | CYS | 201 | 37.847 | 49.848 | 78.912 | 1.00 | 15.68 | DIC |
| ATOM | 1597 | O | CYS | 201 | 37.519 | 49.543 | 77.765 | 1.00 | 15.05 | DIC |
| ATOM | 1598 | N | TYR | 202 | 37.697 | 51.079 | 79.386 | 1.00 | 15.85 | DIC |
| ATOM | 1599 | CA | TYR | 202 | 37.132 | 52.114 | 78.529 | 1.00 | 14.50 | DIC |
| ATOM | 1600 | CB | TYR | 202 | 36.236 | 53.054 | 79.333 | 1.00 | 15.59 | DIC |
| ATOM | 1601 | CG | TYR | 202 | 35.102 | 52.377 | 80.061 | 1.00 | 17.50 | DIC |
| ATOM | 1602 | CD1 | TYR | 202 | 35.173 | 52.162 | 81.434 | 1.00 | 17.93 | DIC |
| ATOM | 1603 | CE1 | TYR | 202 | 34.125 | 51.583 | 82.124 | 1.00 | 19.81 | DIC |
| ATOM | 1604 | CD2 | TYR | 202 | 33.944 | 51.984 | 79.388 | 1.00 | 18.20 | DIC |
| ATOM | 1605 | CE2 | TYR | 202 | 32.882 | 51.396 | 80.072 | 1.00 | 19.70 | DIC |
| ATOM | 1606 | CZ | TYR | 202 | 32.983 | 51.205 | 81.447 | 1.00 | 21.08 | DIC |
| ATOM | 1607 | OH | TYR | 202 | 31.941 | 50.670 | 82.163 | 1.00 | 20.73 | DIC |
| ATOM | 1608 | C | TYR | 202 | 38.206 | 52.933 | 77.831 | 1.00 | 14.93 | DIC |
| ATOM | 1609 | O | TYR | 202 | 39.161 | 53.372 | 78.462 | 1.00 | 15.02 | DIC |
| ATOM | 1610 | N | ALA | 203 | 38.038 | 53.130 | 76.525 | 1.00 | 13.78 | DIC |
| ATOM | 1611 | CA | ALA | 203 | 38.980 | 53.913 | 75.725 | 1.00 | 12.83 | DIC |
| ATOM | 1612 | CB | ALA | 203 | 39.210 | 53.243 | 74.377 | 1.00 | 11.34 | DIC |
| ATOM | 1613 | C | ALA | 203 | 38.402 | 55.306 | 75.514 | 1.00 | 13.01 | DIC |
| ATOM | 1614 | O | ALA | 203 | 37.196 | 55.506 | 75.643 | 1.00 | 12.43 | DIC |
| ATOM | 1615 | N | ASN | 204 | 39.257 | 56.272 | 75.195 | 1.00 | 12.21 | DIC |
| ATOM | 1616 | CA | ASN | 204 | 38.774 | 57.628 | 74.962 | 1.00 | 11.59 | DIC |
| ATOM | 1617 | CB | ASN | 204 | 39.936 | 58.587 | 74.745 | 1.00 | 11.55 | DIC |
| ATOM | 1618 | CG | ASN | 204 | 39.488 | 60.025 | 74.710 | 1.00 | 12.55 | DIC |
| ATOM | 1619 | OD1 | ASN | 204 | 39.185 | 60.613 | 75.752 | 1.00 | 15.02 | DIC |
| ATOM | 1620 | ND2 | ASN | 204 | 39.418 | 60.598 | 73.512 | 1.00 | 8.71 | DIC |
| ATOM | 1621 | C | ASN | 204 | 37.887 | 57.647 | 73.722 | 1.00 | 11.96 | DIC |
| ATOM | 1622 | O | ASN | 204 | 38.075 | 56.844 | 72.803 | 1.00 | 10.62 | DIC |
| ATOM | 1623 | N | SER | 205 | 36.927 | 58.566 | 73.690 | 1.00 | 9.54 | DIC |
| ATOM | 1624 | CA | SER | 205 | 36.031 | 58.653 | 72.550 | 1.00 | 11.60 | DIC |
| ATOM | 1625 | CB | SER | 205 | 34.888 | 59.634 | 72.853 | 1.00 | 11.61 | DIC |
| ATOM | 1626 | OG | SER | 205 | 33.979 | 59.055 | 73.784 | 1.00 | 10.36 | DIC |
| ATOM | 1627 | C | SER | 205 | 36.718 | 59.013 | 71.227 | 1.00 | 11.44 | DIC |
| ATOM | 1628 | O | SER | 205 | 36.099 | 58.920 | 70.166 | 1.00 | 11.98 | DIC |
| ATOM | 1629 | N | ARG | 206 | 37.994 | 59.396 | 71.270 | 1.00 | 10.73 | DIC |
| ATOM | 1630 | CA | ARG | 206 | 38.686 | 59.724 | 70.028 | 1.00 | 10.60 | DIC |
| ATOM | 1631 | CB | ARG | 206 | 40.089 | 60.297 | 70.305 | 1.00 | 10.09 | DIC |
| ATOM | 1632 | CG | ARG | 206 | 41.089 | 59.315 | 70.924 | 1.00 | 11.89 | DIC |
| ATOM | 1633 | CD | ARG | 206 | 42.374 | 60.043 | 71.336 | 1.00 | 12.09 | DIC |
| ATOM | 1634 | NE | ARG | 206 | 42.939 | 60.770 | 70.203 | 1.00 | 12.43 | DIC |
| ATOM | 1635 | CZ | ARG | 206 | 43.984 | 60.367 | 69.484 | 1.00 | 10.79 | DIC |
| ATOM | 1636 | NH1 | ARG | 206 | 44.610 | 59.240 | 69.782 | 1.00 | 11.45 | DIC |
| ATOM | 1637 | NH2 | ARG | 206 | 44.378 | 61.078 | 68.438 | 1.00 | 9.92 | DIC |
| ATOM | 1638 | C | ARG | 206 | 38.789 | 58.473 | 69.149 | 1.00 | 9.80 | DIC |
| ATOM | 1639 | O | ARG | 206 | 38.932 | 58.573 | 67.937 | 1.00 | 9.66 | DIC |
| ATOM | 1640 | N | PHE | 207 | 38.700 | 57.295 | 69.757 | 1.00 | 9.55 | DIC |
| ATOM | 1641 | CA | PHE | 207 | 38.801 | 56.056 | 68.989 | 1.00 | 9.03 | DIC |
| ATOM | 1642 | CB | PHE | 207 | 39.554 | 55.009 | 69.802 | 1.00 | 8.47 | DIC |
| ATOM | 1643 | CG | PHE | 207 | 40.980 | 55.386 | 70.082 | 1.00 | 7.54 | DIC |
| ATOM | 1644 | CD1 | PHE | 207 | 41.388 | 55.703 | 71.368 | 1.00 | 7.40 | DIC |
| ATOM | 1645 | CD2 | PHE | 207 | 41.906 | 55.455 | 69.048 | 1.00 | 7.60 | DIC |
| ATOM | 1646 | CE1 | PHE | 207 | 42.701 | 56.087 | 71.626 | 1.00 | 9.11 | DIC |
| ATOM | 1647 | CE2 | PHE | 207 | 43.217 | 55.837 | 69.290 | 1.00 | 10.35 | DIC |
| ATOM | 1648 | CZ | PHE | 207 | 43.616 | 56.156 | 70.588 | 1.00 | 9.62 | DIC |
| ATOM | 1649 | C | PHE | 207 | 37.472 | 55.488 | 68.495 | 1.00 | 9.66 | DIC |
| ATOM | 1650 | O | PHE | 207 | 37.416 | 54.376 | 67.966 | 1.00 | 10.94 | DIC |
| ATOM | 1651 | N | ASN | 208 | 36.404 | 56.254 | 68.683 | 1.00 | 9.27 | DIC |
| ATOM | 1652 | CA | ASN | 208 | 35.079 | 55.866 | 68.229 | 1.00 | 10.30 | DIC |
| ATOM | 1653 | CB | ASN | 208 | 34.475 | 54.792 | 69.140 | 1.00 | 9.11 | DIC |
| ATOM | 1654 | CG | ASN | 208 | 33.285 | 54.091 | 68.497 | 1.00 | 9.58 | DIC |
| ATOM | 1655 | OD1 | ASN | 208 | 32.668 | 54.626 | 67.573 | 1.00 | 9.75 | DIC |
| ATOM | 1656 | ND2 | ASN | 208 | 32.956 | 52.896 | 68.981 | 1.00 | 5.82 | DIC |
| ATOM | 1657 | C | ASN | 208 | 34.249 | 57.144 | 68.293 | 1.00 | 11.23 | DIC |
| ATOM | 1658 | O | ASN | 208 | 33.407 | 57.303 | 69.172 | 1.00 | 11.29 | DIC |
| ATOM | 1659 | N | PHE | 209 | 34.514 | 58.060 | 67.359 | 1.00 | 11.31 | DIC |
| ATOM | 1660 | CA | PHE | 209 | 33.835 | 59.357 | 67.300 | 1.00 | 11.35 | DIC |
| ATOM | 1661 | CB | PHE | 209 | 34.800 | 60.407 | 66.725 | 1.00 | 10.07 | DIC |
| ATOM | 1662 | CG | PHE | 209 | 34.402 | 61.833 | 67.007 | 1.00 | 10.65 | DIC |
| ATOM | 1663 | CD1 | PHE | 209 | 34.457 | 62.348 | 68.300 | 1.00 | 12.23 | DIC |
| ATOM | 1664 | CD2 | PHE | 209 | 33.991 | 62.669 | 65.972 | 1.00 | 12.03 | DIC |
| ATOM | 1665 | CE1 | PHE | 209 | 34.110 | 63.682 | 68.558 | 1.00 | 14.04 | DIC |
| ATOM | 1666 | CE2 | PHE | 209 | 33.646 | 63.991 | 66.213 | 1.00 | 12.22 | DIC |
| ATOM | 1667 | CZ | PHE | 209 | 33.705 | 64.504 | 67.512 | 1.00 | 13.59 | DIC |
| ATOM | 1668 | C | PHE | 209 | 32.585 | 59.241 | 66.422 | 1.00 | 11.70 | DIC |
| ATOM | 1669 | O | PHE | 209 | 32.676 | 59.193 | 65.198 | 1.00 | 10.06 | DIC |
| ATOM | 1670 | N | MSE | 210 | 31.419 | 59.214 | 67.057 | 1.00 | 11.79 | DIC |
| ATOM | 1671 | CA | MSE | 210 | 30.156 | 59.050 | 66.339 | 1.00 | 13.38 | DIC |
| ATOM | 1672 | CB | MSE | 210 | 29.343 | 57.935 | 67.022 | 1.00 | 13.32 | DIC |
| ATOM | 1673 | CG | MSE | 210 | 30.087 | 56.594 | 67.064 | 1.00 | 14.56 | DIC |
| ATOM | 1674 | SE | MSE | 210 | 29.331 | 55.241 | 68.266 | 1.00 | 14.26 | DIC |
| ATOM | 1675 | CE | MSE | 210 | 30.349 | 55.687 | 69.839 | 1.00 | 11.50 | DIC |
| ATOM | 1676 | C | MSE | 210 | 29.349 | 60.344 | 66.251 | 1.00 | 12.91 | DIC |
| ATOM | 1677 | O | MSE | 210 | 29.739 | 61.369 | 66.815 | 1.00 | 11.87 | DIC |
| ATOM | 1678 | N | PRO | 211 | 28.225 | 60.323 | 65.512 | 1.00 | 14.20 | DIC |
| ATOM | 1679 | CD | PRO | 211 | 27.659 | 59.232 | 64.699 | 1.00 | 12.16 | DIC |
| ATOM | 1680 | CA | PRO | 211 | 27.406 | 61.533 | 65.386 | 1.00 | 14.02 | DIC |
| ATOM | 1681 | CB | PRO | 211 | 26.159 | 61.028 | 64.667 | 1.00 | 13.37 | DIC |
| ATOM | 1682 | CG | PRO | 211 | 26.719 | 59.976 | 63.771 | 1.00 | 14.79 | DIC |
| ATOM | 1683 | C | PRO | 211 | 27.078 | 62.175 | 66.732 | 1.00 | 14.59 | DIC |
| ATOM | 1684 | O | PRO | 211 | 27.129 | 63.401 | 66.873 | 1.00 | 14.82 | DIC |
| ATOM | 1685 | N | THR | 212 | 26.747 | 61.356 | 67.724 | 1.00 | 12.84 | DIC |
| ATOM | 1686 | CA | THR | 212 | 26.415 | 61.915 | 69.026 | 1.00 | 12.38 | DIC |
| ATOM | 1687 | CB | THR | 212 | 25.861 | 60.847 | 69.985 | 1.00 | 11.66 | DIC |
| ATOM | 1688 | OG1 | THR | 212 | 24.754 | 60.185 | 69.367 | 1.00 | 10.96 | DIC |
| ATOM | 1689 | CG2 | THR | 212 | 25.354 | 61.505 | 71.274 | 1.00 | 12.45 | DIC |
| ATOM | 1690 | C | THR | 212 | 27.614 | 62.618 | 69.670 | 1.00 | 13.42 | DIC |
| ATOM | 1691 | O | THR | 212 | 27.448 | 63.662 | 70.307 | 1.00 | 12.42 | DIC |
| ATOM | 1692 | N | ASN | 213 | 28.813 | 62.056 | 69.511 | 1.00 | 11.99 | DIC |
| ATOM | 1693 | CA | ASN | 213 | 30.016 | 62.680 | 70.070 | 1.00 | 11.39 | DIC |
| ATOM | 1694 | CB | ASN | 213 | 31.268 | 61.848 | 69.759 | 1.00 | 10.41 | DIC |
| ATOM | 1695 | CG | ASN | 213 | 31.210 | 60.442 | 70.348 | 1.00 | 9.67 | DIC |
| ATOM | 1696 | OD1 | ASN | 213 | 31.672 | 60.196 | 71.466 | 1.00 | 10.66 | DIC |
| ATOM | 1697 | ND2 | ASN | 213 | 30.637 | 59.515 | 69.595 | 1.00 | 7.33 | DIC |
| ATOM | 1698 | C | ASN | 213 | 30.155 | 64.050 | 69.412 | 1.00 | 13.60 | DIC |
| ATOM | 1699 | O | ASN | 213 | 30.443 | 65.045 | 70.076 | 1.00 | 13.49 | DIC |
| ATOM | 1700 | N | TYR | 214 | 29.948 | 64.093 | 68.097 | 1.00 | 13.25 | DIC |
| ATOM | 1701 | CA | TYR | 214 | 30.042 | 65.345 | 67.358 | 1.00 | 16.71 | DIC |
| ATOM | 1702 | CB | TYR | 214 | 29.790 | 65.116 | 65.863 | 1.00 | 17.70 | DIC |
| ATOM | 1703 | CG | TYR | 214 | 29.918 | 66.380 | 65.041 | 1.00 | 21.47 | DIC |
| ATOM | 1704 | CD1 | TYR | 214 | 31.165 | 66.831 | 64.609 | 1.00 | 23.78 | DIC |
| ATOM | 1705 | CE1 | TYR | 214 | 31.297 | 68.018 | 63.888 | 1.00 | 24.82 | DIC |
| ATOM | 1706 | CD2 | TYP | 214 | 28.798 | 67.147 | 64.732 | 1.00 | 24.77 | DIC |
| ATOM | 1707 | CE2 | TYR | 214 | 28.915 | 68.339 | 64.013 | 1.00 | 26.67 | DIC |
| ATOM | 1708 | CZ | TYR | 214 | 30.168 | 68.768 | 63.594 | 1.00 | 27.64 | DIC |
| ATOM | 1709 | OH | TYR | 214 | 30.291 | 69.949 | 62.885 | 1.00 | 28.93 | DIC |
| ATOM | 1710 | C | TYR | 214 | 29.014 | 66.340 | 67.894 | 1.00 | 18.21 | DIC |
| ATOM | 1711 | O | TYR | 214 | 29.348 | 67.483 | 68.209 | 1.00 | 16.24 | DIC |
| ATOM | 1712 | N | ALA | 215 | 27.759 | 65.906 | 67.985 | 1.00 | 18.99 | DIC |
| ATOM | 1713 | CA | ALA | 215 | 26.700 | 66.774 | 68.488 | 1.00 | 21.66 | DIC |
| ATOM | 1714 | CB | ALA | 215 | 25.379 | 66.021 | 68.547 | 1.00 | 19.99 | DIC |
| ATOM | 1715 | C | ALA | 215 | 27.096 | 67.252 | 69.877 | 1.00 | 24.02 | DIC |
| ATOM | 1716 | O | ALA | 215 | 26.776 | 68.374 | 70.277 | 1.00 | 23.13 | DIC |
| ATOM | 1717 | N | PHE | 216 | 27.802 | 66.397 | 70.609 | 1.00 | 26.12 | DIC |
| ATOM | 1718 | CA | PHE | 216 | 28.240 | 66.755 | 71.945 | 1.00 | 31.30 | DIC |
| ATOM | 1719 | CB | PHE | 216 | 28.883 | 65.562 | 72.648 | 1.00 | 32.05 | DIC |
| ATOM | 1720 | CG | PHE | 216 | 29.073 | 65.769 | 74.123 | 1.00 | 34.39 | DIC |
| ATOM | 1721 | CD1 | PHE | 216 | 27.973 | 65.938 | 74.960 | 1.00 | 34.65 | DIC |
| ATOM | 1722 | CD2 | PHE | 216 | 30.350 | 65.808 | 74.677 | 1.00 | 33.95 | DIC |
| ATOM | 1723 | CE1 | PHE | 216 | 28.141 | 66.146 | 76.328 | 1.00 | 34.98 | DIC |
| ATOM | 1724 | CE2 | PHE | 216 | 30.528 | 66.016 | 76.046 | 1.00 | 35.12 | DIC |
| ATOM | 1725 | CZ | PHE | 216 | 29.422 | 66.186 | 76.871 | 1.00 | 34.75 | DIC |
| ATOM | 1726 | C | PHE | 216 | 29.237 | 67.905 | 71.839 | 1.00 | 34.43 | DIC |
| ATOM | 1727 | O | PHE | 216 | 29.444 | 68.639 | 72.802 | 1.00 | 34.68 | DIC |
| ATOM | 1728 | N | MSE | 217 | 29.862 | 68.048 | 70.670 | 1.00 | 37.59 | DIC |
| ATOM | 1729 | CA | MSE | 217 | 30.794 | 69.152 | 70.445 | 1.00 | 40.37 | DIC |
| ATOM | 1730 | CB | MSE | 217 | 31.572 | 68.981 | 69.134 | 1.00 | 42.90 | DIC |
| ATOM | 1731 | CG | MSE | 217 | 32.503 | 67.782 | 69.051 | 1.00 | 46.79 | DIC |
| ATOM | 1732 | SE | MSE | 217 | 33.962 | 67.801 | 70.312 | 1.00 | 54.87 | DIC |
| ATOM | 1733 | CE | MSE | 217 | 34.812 | 69.461 | 69.818 | 1.00 | 51.10 | DIC |
| ATOM | 1734 | C | MSE | 217 | 29.898 | 70.381 | 70.329 | 1.00 | 40.97 | DIC |
| ATOM | 1735 | O | MSE | 217 | 29.756 | 70.961 | 69.249 | 1.00 | 41.56 | DIC |
| ATOM | 1736 | N | ALA | 218 | 29.278 | 70.748 | 71.446 | 1.00 | 41.05 | DIC |
| ATOM | 1737 | CA | ALA | 218 | 28.374 | 71.890 | 71.526 | 1.00 | 40.94 | DIC |
| ATOM | 1738 | CB | ALA | 218 | 27.354 | 71.852 | 70.384 | 1.00 | 40.11 | DIC |
| ATOM | 1739 | C | ALA | 218 | 27.662 | 71.818 | 72.877 | 1.00 | 40.36 | DIC |
| ATOM | 1740 | OT1 | ALA | 218 | 26.415 | 71.718 | 72.922 | 1.00 | 18.66 | DIC |
| ATOM | 1741 | OT2 | ALA | 218 | 28.375 | 71.852 | 73.894 | 1.00 | 18.66 | DIC |
| ATOM | 1742 | CB | ALA | 222 | 25.959 | 69.432 | 79.985 | 1.00 | 40.17 | DIC |
| ATOM | 1743 | C | ALA | 222 | 25.843 | 67.067 | 79.228 | 1.00 | 40.29 | DIC |
| ATOM | 1744 | O | ALA | 222 | 24.922 | 66.318 | 78.894 | 1.00 | 42.05 | DIC |
| ATOM | 1745 | N | ALA | 222 | 24.590 | 68.826 | 78.056 | 1.00 | 38.20 | DIC |
| ATOM | 1746 | CA | ALA | 222 | 25.844 | 68.531 | 78.798 | 1.00 | 39.59 | DIC |
| ATOM | 1747 | N | SER | 223 | 26.884 | 66.669 | 79.959 | 1.00 | 39.44 | DIC |
| ATOM | 1748 | CA | SER | 223 | 27.149 | 65.301 | 80.439 | 1.00 | 38.81 | DIC |
| ATOM | 1749 | CB | SER | 223 | 28.411 | 65.284 | 81.274 | 1.00 | 39.72 | DIC |
| ATOM | 1750 | OG | SER | 223 | 28.906 | 63.978 | 81.237 | 1.00 | 41.10 | DIC |
| ATOM | 1751 | C | SER | 223 | 26.135 | 64.549 | 81.278 | 1.00 | 37.39 | DIC |
| ATOM | 1752 | O | SER | 223 | 25.489 | 65.136 | 82.124 | 1.00 | 36.31 | DIC |
| ATOM | 1753 | N | ARG | 224 | 26.103 | 63.225 | 81.092 | 1.00 | 35.52 | DIC |
| ATOM | 1754 | CA | ARG | 224 | 25.222 | 62.245 | 81.777 | 1.00 | 34.47 | DIC |
| ATOM | 1755 | CB | ARG | 224 | 25.124 | 60.971 | 80.896 | 1.00 | 33.95 | DIC |
| ATOM | 1756 | CG | ARG | 224 | 23.749 | 60.549 | 80.430 | 1.00 | 35.88 | DIC |
| ATOM | 1757 | CD | ARG | 224 | 23.144 | 59.698 | 81.482 | 1.00 | 36.44 | DIC |
| ATOM | 1758 | NE | ARG | 224 | 21.699 | 59.611 | 81.371 | 1.00 | 37.64 | DIC |
| ATOM | 1759 | CZ | ARG | 224 | 20.913 | 59.130 | 82.315 | 1.00 | 38.47 | DIC |
| ATOM | 1760 | NH1 | ARG | 224 | 19.608 | 59.097 | 82.127 | 1.00 | 38.82 | DIC |
| ATOM | 1761 | NH2 | ARG | 224 | 21.445 | 58.665 | 83.422 | 1.00 | 39.33 | DIC |
| ATOM | 1762 | C | ARG | 224 | 25.652 | 61.827 | 83.196 | 1.00 | 32.76 | DIC |
| ATOM | 1763 | O | ARG | 224 | 24.831 | 61.475 | 84.013 | 1.00 | 33.35 | DIC |
| ATOM | 1764 | N | HIS | 225 | 26.950 | 61.845 | 83.467 | 1.00 | 31.17 | DIC |
| ATOM | 1765 | CA | HIS | 225 | 27.465 | 61.469 | 84.792 | 1.00 | 28.74 | DIC |
| ATOM | 1766 | CB | HIS | 225 | 28.059 | 60.055 | 84.761 | 1.00 | 28.53 | DIC |
| ATOM | 1767 | CG | HIS | 225 | 27.099 | 59.000 | 84.295 | 1.00 | 28.65 | DIC |
| ATOM | 1768 | CD2 | HIS | 225 | 26.931 | 58.416 | 83.083 | 1.00 | 28.14 | DIC |
| ATOM | 1769 | ND1 | HIS | 225 | 26.140 | 58.451 | 85.118 | 1.00 | 26.79 | DIC |
| ATOM | 1770 | CE1 | HIS | 225 | 25.422 | 57.576 | 84.435 | 1.00 | 27.52 | DIC |
| ATOM | 1771 | NE2 | HIS | 225 | 25.881 | 57.537 | 83.197 | 1.00 | 27.77 | DIC |
| ATOM | 1772 | C | HIS | 225 | 28.535 | 62.441 | 85.283 | 1.00 | 28.61 | DIC |
| ATOM | 1773 | O | HIS | 225 | 29.209 | 63.061 | 84.468 | 1.00 | 28.11 | DIC |
| ATOM | 1774 | N | THR | 226 | 28.700 | 62.567 | 86.600 | 1.00 | 26.51 | DIC |
| ATOM | 1775 | CA | THR | 226 | 29.713 | 63.466 | 87.150 | 1.00 | 24.55 | DIC |
| ATOM | 1776 | CB | THR | 226 | 29.241 | 64.076 | 88.488 | 1.00 | 24.86 | DIC |
| ATOM | 1777 | OG1 | THR | 226 | 28.907 | 63.025 | 89.402 | 1.00 | 25.94 | DIC |
| ATOM | 1778 | CG2 | THR | 226 | 28.010 | 64.942 | 88.268 | 1.00 | 24.89 | DIC |
| ATOM | 1779 | C | THR | 226 | 31.007 | 62.678 | 87.360 | 1.00 | 23.42 | DIC |
| ATOM | 1780 | O | THR | 226 | 32.091 | 63.247 | 87.525 | 1.00 | 22.81 | DIC |
| ATOM | 1781 | N | ASP | 227 | 30.875 | 61.353 | 87.352 | 1.00 | 21.74 | DIC |
| ATOM | 1782 | CA | ASP | 227 | 32.014 | 60.456 | 87.515 | 1.00 | 20.74 | DIC |
| ATOM | 1783 | CB | ASP | 227 | 31.634 | 59.036 | 87.073 | 1.00 | 20.56 | DIC |
| ATOM | 1784 | CG | ASP | 227 | 32.817 | 58.091 | 87.067 | 1.00 | 20.53 | DIC |
| ATOM | 1785 | OD1 | ASP | 227 | 32.691 | 56.986 | 86.495 | 1.00 | 22.53 | DIC |
| ATOM | 1786 | OD2 | ASP | 227 | 33.872 | 58.442 | 87.633 | 1.00 | 21.73 | DIC |
| ATOM | 1787 | C | ASP | 227 | 33.188 | 60.957 | 86.672 | 1.00 | 20.76 | DIC |
| ATOM | 1788 | O | ASP | 227 | 33.118 | 60.985 | 85.440 | 1.00 | 17.27 | DIC |
| ATOM | 1789 | N | PRO | 228 | 34.288 | 61.351 | 87.331 | 1.00 | 21.29 | DIC |
| ATOM | 1790 | CD | PRO | 228 | 34.547 | 61.210 | 88.776 | 1.00 | 22.76 | DIC |
| ATOM | 1791 | CA | PRO | 228 | 35.476 | 61.853 | 86.634 | 1.00 | 21.24 | DIC |
| ATOM | 1792 | CB | PRO | 228 | 36.476 | 62.076 | 87.772 | 1.00 | 23.32 | DIC |
| ATOM | 1793 | CG | PRO | 228 | 36.047 | 61.074 | 88.809 | 1.00 | 24.69 | DIC |
| ATOM | 1794 | C | PRO | 228 | 36.010 | 60.921 | 85.549 | 1.00 | 21.24 | DIC |
| ATOM | 1795 | O | PRO | 228 | 36.503 | 61.383 | 84.518 | 1.00 | 20.14 | DIC |
| ATOM | 1796 | N | LEU | 229 | 35.913 | 59.612 | 85.766 | 1.00 | 20.48 | DIC |
| ATOM | 1797 | CA | LEU | 229 | 36.397 | 58.677 | 84.758 | 1.00 | 19.34 | DIC |
| ATOM | 1798 | CB | LEU | 229 | 36.439 | 57.257 | 85.318 | 1.00 | 20.42 | DIC |
| ATOM | 1799 | CG | LEU | 229 | 36.953 | 56.172 | 84.371 | 1.00 | 22.18 | DIC |
| ATOM | 1800 | CD1 | LEU | 229 | 37.630 | 55.069 | 85.167 | 1.00 | 22.60 | DIC |
| ATOM | 1801 | CD2 | LEU | 229 | 35.792 | 55.624 | 83.545 | 1.00 | 20.97 | DIC |
| ATOM | 1802 | C | LEU | 229 | 35.502 | 58.739 | 83.526 | 1.00 | 17.99 | DIC |
| ATOM | 1803 | O | LEU | 229 | 35.990 | 58.809 | 82.397 | 1.00 | 17.37 | DIC |
| ATOM | 1804 | N | TYR | 230 | 34.192 | 58.723 | 83.746 | 1.00 | 18.30 | DIC |
| ATOM | 1805 | CA | TYR | 230 | 33.234 | 58.796 | 82.647 | 1.00 | 17.44 | DIC |
| ATOM | 1806 | CB | TYR | 230 | 31.799 | 58.862 | 83.189 | 1.00 | 18.25 | DIC |
| ATOM | 1807 | CG | TYR | 230 | 30.741 | 58.971 | 82.109 | 1.00 | 16.68 | DIC |
| ATOM | 1808 | CD1 | TYR | 230 | 30.333 | 57.850 | 81.385 | 1.00 | 18.08 | DIC |
| ATOM | 1809 | CE1 | TYR | 230 | 29.388 | 57.953 | 80.370 | 1.00 | 16.04 | DIC |
| ATOM | 1810 | CD2 | TYR | 230 | 30.173 | 60.202 | 81.788 | 1.00 | 16.80 | DIC |
| ATOM | 1811 | CE2 | TYR | 230 | 29.232 | 60.320 | 80.777 | 1.00 | 16.57 | DIC |
| ATOM | 1812 | CZ | TYR | 230 | 28.842 | 59.192 | 80.070 | 1.00 | 17.59 | DIC |
| ATOM | 1813 | OH | TYR | 230 | 27.908 | 59.314 | 79.067 | 1.00 | 13.29 | DIC |
| ATOM | 1814 | C | TYR | 230 | 33.520 | 60.049 | 81.818 | 1.00 | 18.05 | DIC |
| ATOM | 1815 | O | TYR | 230 | 33.575 | 59.996 | 80.588 | 1.00 | 17.20 | DIC |
| ATOM | 1816 | N | ARG | 231 | 33.704 | 61.174 | 82.504 | 1.00 | 17.40 | DIC |
| ATOM | 1817 | CA | ARG | 231 | 33.979 | 62.438 | 81.829 | 1.00 | 18.57 | DIC |
| ATOM | 1818 | CB | ARG | 231 | 33.948 | 63.585 | 82.841 | 1.00 | 21.41 | DIC |
| ATOM | 1819 | CG | ARG | 231 | 32.535 | 63.903 | 83.336 | 1.00 | 27.36 | DIC |
| ATOM | 1820 | CD | ARG | 231 | 32.562 | 64.595 | 84.685 | 1.00 | 32.89 | DIC |
| ATOM | 1821 | NE | ARG | 231 | 33.446 | 65.755 | 84.670 | 1.00 | 39.04 | DIC |
| ATOM | 1822 | CZ | ARG | 231 | 33.229 | 66.848 | 83.946 | 1.00 | 41.47 | DIC |
| ATOM | 1823 | NH1 | ARG | 231 | 34.092 | 67.858 | 83.988 | 1.00 | 42.86 | DIC |
| ATOM | 1824 | NH2 | ARG | 231 | 32.142 | 66.936 | 83.187 | 1.00 | 43.95 | DIC |
| ATOM | 1825 | C | ARG | 231 | 35.307 | 62.420 | 81.079 | 1.00 | 18.13 | DIC |
| ATOM | 1826 | O | ARG | 231 | 35.407 | 62.943 | 79.972 | 1.00 | 16.25 | DIC |
| ATOM | 1827 | N | ASP | 232 | 36.328 | 61.821 | 81.678 | 1.00 | 16.04 | DIC |
| ATOM | 1828 | CA | ASP | 232 | 37.620 | 61.749 | 81.021 | 1.00 | 17.65 | DIC |
| ATOM | 1829 | CB | ASP | 232 | 38.646 | 61.092 | 81.948 | 1.00 | 19.41 | DIC |
| ATOM | 1830 | CG | ASP | 232 | 39.972 | 60.839 | 81.258 | 1.00 | 22.20 | DIC |
| ATOM | 1831 | OD1 | ASP | 232 | 40.137 | 59.760 | 80.648 | 1.00 | 24.13 | DIC |
| ATOM | 1832 | OD2 | ASP | 232 | 40.845 | 61.729 | 81.314 | 1.00 | 24.43 | DIC |
| ATOM | 1833 | C | ASP | 232 | 37.522 | 60.957 | 79.713 | 1.00 | 17.38 | DIC |
| ATOM | 1834 | O | ASP | 232 | 38.078 | 61.359 | 78.690 | 1.00 | 16.38 | DIC |
| ATOM | 1835 | N | ARG | 233 | 36.789 | 59.844 | 79.746 | 1.00 | 15.90 | DIC |
| ATOM | 1836 | CA | ARG | 233 | 36.647 | 58.992 | 78.574 | 1.00 | 14.22 | DIC |
| ATOM | 1837 | CB | ARG | 233 | 36.134 | 57.609 | 78.993 | 1.00 | 13.65 | DIC |
| ATOM | 1838 | CG | ARG | 233 | 36.992 | 56.898 | 80.042 | 1.00 | 12.06 | DIC |
| ATOM | 1839 | CD | ARG | 233 | 38.359 | 56.471 | 79.507 | 1.00 | 11.93 | DIC |
| ATOM | 1840 | NE | ARG | 233 | 38.994 | 55.502 | 80.393 | 1.00 | 12.99 | DIC |
| ATOM | 1841 | CZ | ARG | 233 | 39.587 | 55.800 | 81.549 | 1.00 | 18.67 | DIC |
| ATOM | 1842 | NH1 | ARG | 233 | 39.647 | 57.056 | 81.976 | 1.00 | 15.62 | DIC |
| ATOM | 1843 | NH2 | ARG | 233 | 40.096 | 54.829 | 82.301 | 1.00 | 19.37 | DIC |
| ATOM | 1844 | C | ARG | 233 | 35.745 | 59.560 | 77.477 | 1.00 | 14.94 | DIC |
| ATOM | 1845 | O | ARG | 233 | 36.042 | 59.407 | 76.291 | 1.00 | 13.66 | DIC |
| ATOM | 1846 | N | THR | 234 | 34.656 | 60.222 | 77.867 | 1.00 | 14.04 | DIC |
| ATOM | 1847 | CA | THR | 234 | 33.716 | 60.772 | 76.895 | 1.00 | 14.66 | DIC |
| ATOM | 1848 | CB | THR | 234 | 32.298 | 60.886 | 77.513 | 1.00 | 15.37 | DIC |
| ATOM | 1849 | OG1 | TSP | 234 | 32.365 | 61.614 | 78.745 | 1.00 | 12.97 | DIC |
| ATOM | 1850 | CG2 | THR | 234 | 31.725 | 59.474 | 77.789 | 1.00 | 14.09 | DIC |
| ATOM | 1851 | C | THR | 234 | 34.149 | 62.114 | 76.298 | 1.00 | 16.57 | DIC |
| ATOM | 1852 | O | THR | 234 | 33.618 | 62.555 | 75.282 | 1.00 | 15.27 | DIC |
| ATOM | 1853 | N | ASN | 235 | 35.115 | 62.767 | 76.926 | 1.00 | 16.09 | DIC |
| ATOM | 1854 | CA | ASN | 235 | 35.594 | 64.017 | 76.386 | 1.00 | 18.20 | DIC |
| ATOM | 1855 | CB | ASN | 235 | 36.034 | 64.941 | 77.519 | 1.00 | 22.89 | DIC |
| ATOM | 1856 | CG | ASN | 235 | 34.843 | 65.640 | 78.171 | 1.00 | 28.06 | DIC |
| ATOM | 1857 | OD1 | ASN | 235 | 34.410 | 66.700 | 77.716 | 1.00 | 32.63 | DIC |
| ATOM | 1858 | ND2 | ASN | 235 | 34.282 | 65.029 | 79.210 | 1.00 | 30.92 | DIC |
| ATOM | 1859 | C | ASN | 235 | 36.708 | 63.648 | 75.423 | 1.00 | 17.06 | DIC |
| ATOM | 1860 | O | ASN | 235 | 37.793 | 63.214 | 75.810 | 1.00 | 14.88 | DIC |
| ATOM | 1861 | N | THR | 236 | 36.389 | 63.780 | 74.142 | 1.00 | 16.31 | DIC |
| ATOM | 1862 | CA | THR | 236 | 37.308 | 63.421 | 73.084 | 1.00 | 14.87 | DIC |
| ATOM | 1863 | CB | THR | 236 | 36.623 | 63.547 | 71.714 | 1.00 | 15.58 | DIC |
| ATOM | 1864 | OG1 | THR | 236 | 35.566 | 62.578 | 71.629 | 1.00 | 14.42 | DIC |
| ATOM | 1865 | CG2 | THR | 236 | 37.619 | 63.309 | 70.588 | 1.00 | 13.74 | DIC |
| ATOM | 1866 | C | THR | 236 | 38.587 | 64.219 | 73.088 | 1.00 | 15.17 | DIC |
| ATOM | 1867 | O | THR | 236 | 38.567 | 65.454 | 73.120 | 1.00 | 15.92 | DIC |
| ATOM | 1868 | N | VAL | 237 | 39.706 | 63.503 | 73.086 | 1.00 | 13.08 | DIC |
| ATOM | 1869 | CA | VAL | 237 | 41.006 | 64.150 | 73.049 | 1.00 | 14.84 | DIC |
| ATOM | 1870 | CB | VAL | 237 | 42.080 | 63.350 | 73.818 | 1.00 | 16.34 | DIC |
| ATOM | 1871 | CG1 | VAL | 237 | 43.444 | 64.022 | 73.635 | 1.00 | 16.24 | DIC |
| ATOM | 1872 | CG2 | VAL | 237 | 41.723 | 63.279 | 75.308 | 1.00 | 16.15 | DIC |
| ATOM | 1873 | C | VAL | 237 | 41.388 | 64.205 | 71.574 | 1.00 | 14.29 | DIC |
| ATOM | 1874 | O | VAL | 237 | 41.667 | 63.174 | 70.954 | 1.00 | 13.91 | DIC |
| ATOM | 1875 | N | MSE | 238 | 41.380 | 65.408 | 71.015 | 1.00 | 14.48 | DIC |
| ATOM | 1876 | CA | MSE | 238 | 41.710 | 65.608 | 69.603 | 1.00 | 15.10 | DIC |
| ATOM | 1877 | CB | MSE | 238 | 41.462 | 67.065 | 69.223 | 1.00 | 16.28 | DIC |
| ATOM | 1878 | CG | MSE | 238 | 40.012 | 67.495 | 69.341 | 1.00 | 20.30 | DIC |
| ATOM | 1879 | SE | MSE | 238 | 38.859 | 66.527 | 68.131 | 1.00 | 22.47 | DIC |
| ATOM | 1880 | CE | MSE | 238 | 37.180 | 66.780 | 69.061 | 1.00 | 25.99 | DIC |
| ATOM | 1881 | C | MSE | 238 | 43.160 | 65.234 | 69.288 | 1.00 | 13.91 | DIC |
| ATOM | 1882 | O | MSE | 238 | 44.021 | 65.289 | 70.163 | 1.00 | 14.24 | DIC |
| ATOM | 1883 | N | PRO | 239 | 43.451 | 64.872 | 68.022 | 1.00 | 14.10 | DIC |
| ATOM | 1884 | CD | PRO | 239 | 44.832 | 64.616 | 67.570 | 1.00 | 14.78 | DIC |
| ATOM | 1885 | CA | PRO | 239 | 42.515 | 64.788 | 66.895 | 1.00 | 13.37 | DIC |
| ATOM | 1886 | CB | PRO | 239 | 43.438 | 64.848 | 65.682 | 1.00 | 14.16 | DIC |
| ATOM | 1887 | CG | PRO | 239 | 44.630 | 64.088 | 66.154 | 1.00 | 15.87 | DIC |
| ATOM | 1888 | C | PRO | 239 | 41.696 | 63.501 | 66.914 | 1.00 | 13.85 | DIC |
| ATOM | 1889 | O | PRO | 239 | 42.065 | 62.530 | 67.582 | 1.00 | 12.56 | DIC |
| ATOM | 1890 | N | VAL | 240 | 40.588 | 63.490 | 66.179 | 1.00 | 12.87 | DIC |
| ATOM | 1891 | CA | VAL | 240 | 39.758 | 62.296 | 66.118 | 1.00 | 12.68 | DIC |
| ATOM | 1892 | CB | VAL | 240 | 38.485 | 62.538 | 65.260 | 1.00 | 13.59 | DIC |
| ATOM | 1893 | CG1 | VAL | 240 | 37.735 | 61.231 | 65.054 | 1.00 | 15.84 | DIC |
| ATOM | 1894 | CG2 | VAL | 240 | 37.574 | 63.557 | 65.962 | 1.00 | 13.32 | DIC |
| ATOM | 1895 | C | VAL | 240 | 40.615 | 61.210 | 65.494 | 1.00 | 11.91 | DIC |
| ATOM | 1896 | O | VAL | 240 | 41.331 | 61.470 | 64.526 | 1.00 | 12.36 | DIC |
| ATOM | 1897 | N | ALA | 241 | 40.566 | 60.003 | 66.054 | 1.00 | 10.60 | DIC |
| ATOM | 1898 | CA | ALA | 241 | 41.366 | 58.887 | 65.529 | 1.00 | 9.24 | DIC |
| ATOM | 1899 | CB | ALA | 241 | 41.989 | 58.107 | 66.688 | 1.00 | 8.43 | DIC |
| ATOM | 1900 | C | ALA | 241 | 40.515 | 57.960 | 64.663 | 1.00 | 9.78 | DIC |
| ATOM | 1901 | O | ALA | 241 | 40.990 | 57.400 | 63.673 | 1.00 | 9.54 | DIC |
| ATOM | 1902 | N | VAL | 242 | 39.250 | 57.804 | 65.042 | 1.00 | 8.78 | DIC |
| ATOM | 1903 | CA | VAL | 242 | 38.327 | 56.961 | 64.296 | 1.00 | 8.01 | DIC |
| ATOM | 1904 | CB | VAL | 242 | 38.120 | 55.590 | 64.998 | 1.00 | 8.78 | DIC |
| ATOM | 1905 | CG1 | VAL | 242 | 37.181 | 54.717 | 64.170 | 1.00 | 7.09 | DIC |
| ATOM | 1906 | CG2 | VAL | 242 | 39.458 | 54.896 | 65.195 | 1.00 | 7.39 | DIC |
| ATOM | 1907 | C | VAL | 242 | 36.955 | 57.604 | 64.163 | 1.00 | 9.79 | DIC |
| ATOM | 1908 | O | VAL | 242 | 36.311 | 57.906 | 65.170 | 1.00 | 9.84 | DIC |
| ATOM | 1909 | N | SER | 243 | 36.521 | 57.830 | 62.924 | 1.00 | 10.04 | DIC |
| ATOM | 1910 | CA | SER | 243 | 35.194 | 58.371 | 62.658 | 1.00 | 11.61 | DIC |
| ATOM | 1911 | CB | SER | 243 | 35.153 | 59.147 | 61.331 | 1.00 | 12.32 | DIC |
| ATOM | 1912 | OG | SER | 243 | 35.555 | 60.495 | 61.486 | 1.00 | 16.91 | DIC |
| ATOM | 1913 | C | SER | 243 | 34.344 | 57.115 | 62.509 | 1.00 | 12.31 | DIC |
| ATOM | 1914 | O | SER | 243 | 34.747 | 56.183 | 61.820 | 1.00 | 13.73 | DIC |
| ATOM | 1915 | N | HIS | 244 | 33.189 | 57.078 | 63.165 | 1.00 | 11.25 | DIC |
| ATOM | 1916 | CA | HIS | 244 | 32.297 | 55.925 | 63.079 | 1.00 | 9.29 | DIC |
| ATOM | 1917 | CB | HIS | 244 | 32.266 | 55.187 | 64.429 | 1.00 | 7.53 | DIC |
| ATOM | 1918 | CG | HIS | 244 | 31.407 | 53.958 | 64.434 | 1.00 | 8.43 | DIC |
| ATOM | 1919 | CD2 | HIS | 244 | 30.813 | 53.278 | 63.424 | 1.00 | 7.51 | DIC |
| ATOM | 1920 | ND1 | HIS | 244 | 31.055 | 53.302 | 65.598 | 1.00 | 6.41 | DIC |
| ATOM | 1921 | CE1 | HIS | 244 | 30.277 | 52.274 | 65.300 | 1.00 | 8.12 | DIC |
| ATOM | 1922 | NE2 | HIS | 244 | 30.114 | 52.238 | 63.990 | 1.00 | 6.81 | DIC |
| ATOM | 1923 | C | HIS | 244 | 30.903 | 56.446 | 62.710 | 1.00 | 9.33 | DIC |
| ATOM | 1924 | O | HIS | 244 | 30.278 | 57.173 | 63.477 | 1.00 | 9.05 | DIC |
| ATOM | 1925 | N | TYR | 245 | 30.423 | 56.073 | 61.530 | 1.00 | 9.10 | DIC |
| ATOM | 1926 | CA | TYR | 245 | 29.120 | 56.524 | 61.051 | 1.00 | 10.38 | DIC |
| ATOM | 1927 | CB | TYR | 245 | 29.213 | 56.762 | 59.545 | 1.00 | 11.80 | DIC |
| ATOM | 1928 | CG | TYR | 245 | 30.308 | 57.747 | 59.216 | 1.00 | 13.82 | DIC |
| ATOM | 1929 | CD1 | TYR | 245 | 30.092 | 59.118 | 59.344 | 1.00 | 14.45 | DIC |
| ATOM | 1930 | CE1 | TYR | 245 | 31.127 | 60.035 | 59.150 | 1.00 | 16.51 | DIC |
| ATOM | 1931 | CD2 | TYR | 245 | 31.591 | 57.308 | 58.875 | 1.00 | 14.05 | DIC |
| ATOM | 1932 | CE2 | TYR | 245 | 32.636 | 58.217 | 58.679 | 1.00 | 15.80 | DIC |
| ATOM | 1933 | CZ | TYR | 245 | 32.396 | 59.579 | 58.822 | 1.00 | 16.33 | DIC |
| ATOM | 1934 | OH | TYR | 245 | 33.422 | 60.487 | 58.666 | 1.00 | 15.53 | DIC |
| ATOM | 1935 | C | TYR | 245 | 27.991 | 55.552 | 61.380 | 1.00 | 11.30 | DIC |
| ATOM | 1936 | O | TYR | 245 | 27.281 | 55.062 | 60.490 | 1.00 | 10.36 | DIC |
| ATOM | 1937 | N | CYS | 246 | 27.818 | 55.284 | 62.670 | 1.00 | 11.34 | DIC |
| ATOM | 1938 | CA | CYS | 246 | 26.781 | 54.363 | 63.101 | 1.00 | 13.80 | DIC |
| ATOM | 1939 | CB | CYS | 246 | 26.886 | 54.103 | 64.605 | 1.00 | 15.97 | DIC |
| ATOM | 1940 | SG | CYS | 246 | 26.808 | 55.590 | 65.610 | 1.00 | 19.68 | DIC |
| ATOM | 1941 | C | CYS | 246 | 25.422 | 54.947 | 62.763 | 1.00 | 15.08 | DIC |
| ATOM | 1942 | O | CYS | 246 | 25.254 | 56.167 | 62.704 | 1.00 | 14.66 | DIC |
| ATOM | 1943 | N | GLY | 247 | 24.453 | 54.071 | 62.529 | 1.00 | 14.33 | DIC |
| ATOM | 1944 | CA | GLY | 247 | 23.127 | 54.539 | 62.191 | 1.00 | 14.42 | DIC |
| ATOM | 1945 | C | GLY | 247 | 22.893 | 54.396 | 60.703 | 1.00 | 16.40 | DIC |
| ATOM | 1946 | O | GLY | 247 | 23.789 | 53.977 | 59.974 | 1.00 | 14.60 | DIC |
| ATOM | 1947 | N | PRO | 248 | 21.693 | 54.754 | 60.218 | 1.00 | 17.44 | DIC |
| ATOM | 1948 | CD | PRO | 248 | 20.586 | 55.331 | 61.001 | 1.00 | 17.20 | DIC |
| ATOM | 1949 | CA | PRO | 248 | 21.334 | 54.660 | 58.800 | 1.00 | 17.61 | DIC |
| ATOM | 1950 | CB | PRO | 248 | 19.845 | 55.009 | 58.809 | 1.00 | 19.08 | DIC |
| ATOM | 1951 | CG | PRO | 248 | 19.750 | 56.000 | 59.933 | 1.00 | 18.65 | DIC |
| ATOM | 1952 | C | PRO | 248 | 22.132 | 55.545 | 57.833 | 1.00 | 17.78 | DIC |
| ATOM | 1953 | O | PRO | 248 | 22.311 | 55.187 | 56.675 | 1.00 | 18.06 | DIC |
| ATOM | 1954 | N | ALA | 249 | 22.617 | 56.690 | 58.305 | 1.00 | 18.95 | DIC |
| ATOM | 1955 | CA | ALA | 249 | 23.362 | 57.612 | 57.444 | 1.00 | 18.77 | DIC |
| ATOM | 1956 | CB | ALA | 249 | 23.276 | 59.038 | 58.008 | 1.00 | 18.55 | DIC |
| ATOM | 1957 | C | ALA | 249 | 24.823 | 57.206 | 57.266 | 1.00 | 18.31 | DIC |
| ATOM | 1958 | O | ALA | 249 | 25.635 | 57.356 | 58.178 | 1.00 | 19.11 | DIC |
| ATOM | 1959 | N | LYS | 250 | 25.156 | 56.705 | 56.082 | 1.00 | 16.06 | DIC |
| ATOM | 1960 | CA | LYS | 250 | 26.515 | 56.269 | 55.808 | 1.00 | 16.76 | DIC |
| ATOM | 1961 | CB | LYS | 250 | 26.498 | 54.854 | 55.226 | 1.00 | 14.84 | DIC |
| ATOM | 1962 | CG | LYS | 250 | 25.771 | 53.836 | 56.093 | 1.00 | 14.31 | DIC |
| ATOM | 1963 | CD | LYS | 250 | 26.384 | 53.731 | 57.487 | 1.00 | 14.00 | DIC |
| ATOM | 1964 | CE | LYS | 250 | 25.797 | 52.551 | 58.272 | 1.00 | 11.62 | DIC |
| ATOM | 1965 | NZ | LYS | 250 | 26.190 | 52.604 | 59.721 | 1.00 | 9.99 | DIC |
| ATOM | 1966 | C | LYS | 250 | 27.246 | 57.213 | 54.857 | 1.00 | 17.98 | DIC |
| ATOM | 1967 | O | LYS | 250 | 26.621 | 57.890 | 54.036 | 1.00 | 17.45 | DIC |
| ATOM | 1968 | N | PRO | 251 | 28.588 | 57.251 | 54.943 | 1.00 | 18.59 | DIC |
| ATOM | 1969 | CD | PRO | 251 | 29.466 | 56.428 | 55.797 | 1.00 | 19.31 | DIC |
| ATOM | 1970 | CA | PRO | 251 | 29.379 | 58.126 | 54.076 | 1.00 | 19.82 | DIC |
| ATOM | 1971 | CB | PRO | 251 | 30.792 | 57.983 | 54.640 | 1.00 | 18.90 | DIC |
| ATOM | 1972 | CG | PRO | 251 | 30.815 | 56.562 | 55.106 | 1.00 | 19.40 | DIC |
| ATOM | 1973 | C | PRO | 251 | 29.278 | 57.761 | 52.594 | 1.00 | 21.72 | DIC |
| ATOM | 1974 | O | PRO | 251 | 29.544 | 58.602 | 51.731 | 1.00 | 22.19 | DIC |
| ATOM | 1975 | N | TRP | 252 | 28.885 | 56.521 | 52.298 | 1.00 | 22.68 | DIC |
| ATOM | 1976 | CA | TRP | 252 | 28.732 | 56.090 | 50.904 | 1.00 | 24.72 | DIC |
| ATOM | 1977 | CB | TRP | 252 | 29.053 | 54.599 | 50.751 | 1.00 | 22.37 | DIC |
| ATOM | 1978 | CG | TRP | 252 | 28.332 | 53.709 | 51.710 | 1.00 | 17.98 | DIC |
| ATOM | 1979 | CD2 | TRP | 252 | 28.886 | 53.095 | 52.875 | 1.00 | 16.74 | DIC |
| ATOM | 1980 | CE2 | TRP | 252 | 27.856 | 52.345 | 53.485 | 1.00 | 15.49 | DIC |
| ATOM | 1981 | CE3 | TRP | 252 | 30.156 | 53.105 | 53.466 | 1.00 | 14.56 | DIC |
| ATOM | 1982 | CD1 | TRP | 252 | 27.022 | 53.321 | 51.658 | 1.00 | 18.51 | DIC |
| ATOM | 1983 | NE1 | TRP | 252 | 26.728 | 52.500 | 52.722 | 1.00 | 15.35 | DIC |
| ATOM | 1984 | CZ2 | TRP | 252 | 28.058 | 51.613 | 54.657 | 1.00 | 15.42 | DIC |
| ATOM | 1985 | CZ3 | TRP | 252 | 30.356 | 52.378 | 54.630 | 1.00 | 14.20 | DIC |
| ATOM | 1986 | CH2 | TRP | 252 | 29.312 | 51.642 | 55.212 | 1.00 | 14.24 | DIC |
| ATOM | 1987 | C | TRP | 252 | 27.318 | 56.372 | 50.397 | 1.00 | 27.39 | DIC |
| ATOM | 1988 | O | TRP | 252 | 26.969 | 56.026 | 49.266 | 1.00 | 28.26 | DIC |
| ATOM | 1989 | N | HIS | 253 | 26.512 | 56.996 | 51.249 | 1.00 | 30.21 | DIC |
| ATOM | 1990 | CA | HIS | 253 | 25.140 | 57.358 | 50.909 | 1.00 | 34.07 | DIC |
| ATOM | 1991 | CB | HIS | 253 | 24.228 | 57.315 | 52.138 | 1.00 | 34.17 | DIC |
| ATOM | 1992 | CG | HIS | 253 | 23.745 | 55.947 | 52.498 | 1.00 | 34.88 | DIC |
| ATOM | 1993 | CD2 | HIS | 253 | 23.210 | 55.469 | 53.644 | 1.00 | 34.49 | DIC |
| ATOM | 1994 | ND1 | HIS | 253 | 23.731 | 54.903 | 51.598 | 1.00 | 33.80 | DIC |
| ATOM | 1995 | CE1 | HIS | 253 | 23.206 | 53.837 | 52.178 | 1.00 | 35.02 | DIC |
| ATOM | 1996 | NE2 | HIS | 253 | 22.881 | 54.152 | 53.419 | 1.00 | 35.95 | DIC |
| ATOM | 1997 | C | HIS | 253 | 25.102 | 58.778 | 50.364 | 1.00 | 36.66 | DIC |
| ATOM | 1998 | O | HIS | 253 | 26.079 | 59.525 | 50.464 | 1.00 | 37.76 | DIC |
| ATOM | 1999 | N | ARG | 254 | 23.954 | 59.144 | 49.803 | 1.00 | 38.76 | DIC |
| ATOM | 2000 | CA | ARG | 254 | 23.751 | 60.476 | 49.253 | 1.00 | 40.22 | DIC |
| ATOM | 2001 | CB | ARG | 254 | 22.605 | 60.449 | 48.238 | 1.00 | 41.70 | DIC |
| ATOM | 2002 | CG | ARG | 254 | 22.678 | 59.273 | 47.266 | 1.00 | 43.79 | DIC |
| ATOM | 2003 | CD | ARG | 254 | 21.435 | 59.178 | 46.392 | 1.00 | 44.39 | DIC |
| ATOM | 2004 | NE | ARG | 254 | 20.205 | 59.068 | 47.174 | 1.00 | 44.17 | DIC |
| ATOM | 2005 | CZ | ARG | 254 | 18.991 | 58.970 | 46.642 | 1.00 | 43.46 | DIC |
| ATOM | 2006 | NH1 | ARG | 254 | 18.844 | 58.969 | 45.323 | 1.00 | 43.96 | DIC |
| ATOM | 2007 | NH2 | ARG | 254 | 17.925 | 58.873 | 47.425 | 1.00 | 42.76 | DIC |
| ATOM | 2008 | C | ARG | 254 | 23.392 | 61.401 | 50.411 | 1.00 | 40.54 | DIC |
| ATOM | 2009 | O | ARG | 254 | 22.796 | 60.969 | 51.401 | 1.00 | 40.03 | DIC |
| ATOM | 2010 | N | ASP | 255 | 23.772 | 62.667 | 50.291 | 1.00 | 40.92 | DIC |
| ATOM | 2011 | CA | ASP | 255 | 23.477 | 63.659 | 51.315 | 1.00 | 41.47 | DIC |
| ATOM | 2012 | CB | ASP | 255 | 21.981 | 63.995 | 51.280 | 1.00 | 42.69 | DIC |
| ATOM | 2013 | CG | ASP | 255 | 21.555 | 64.612 | 49.953 | 1.00 | 43.75 | DIC |
| ATOM | 2014 | OD1 | ASP | 255 | 21.950 | 65.766 | 49.678 | 1.00 | 43.58 | DIC |
| ATOM | 2015 | OD2 | ASP | 255 | 20.835 | 63.939 | 49.181 | 1.00 | 44.62 | DIC |
| ATOM | 2016 | C | ASP | 255 | 23.894 | 63.273 | 52.738 | 1.00 | 41.91 | DIC |
| ATOM | 2017 | O | ASP | 255 | 23.144 | 63.491 | 53.694 | 1.00 | 41.85 | DIC |
| ATOM | 2018 | N | CYS | 256 | 25.086 | 62.696 | 52.882 | 1.00 | 41.35 | DIC |
| ATOM | 2019 | CA | CYS | 256 | 25.574 | 62.338 | 54.211 | 1.00 | 39.96 | DIC |
| ATOM | 2020 | CB | CYS | 256 | 26.664 | 61.266 | 54.133 | 1.00 | 39.72 | DIC |
| ATOM | 2021 | SG | CYS | 256 | 27.207 | 60.674 | 55.763 | 1.00 | 40.45 | DIC |
| ATOM | 2022 | C | CYS | 256 | 26.150 | 63.623 | 54.800 | 1.00 | 38.56 | DIC |
| ATOM | 2023 | O | CYS | 256 | 27.114 | 64.177 | 54.273 | 1.00 | 37.54 | DIC |
| ATOM | 2024 | N | THR | 257 | 25.547 | 64.103 | 55.883 | 1.00 | 38.47 | DIC |
| ATOM | 2025 | CA | THR | 257 | 25.996 | 65.339 | 56.515 | 1.00 | 36.95 | DIC |
| ATOM | 2026 | CB | THR | 257 | 24.795 | 66.183 | 56.963 | 1.00 | 37.26 | DIC |
| ATOM | 2027 | OG1 | THR | 257 | 24.065 | 65.475 | 57.974 | 1.00 | 37.57 | DIC |
| ATOM | 2028 | CG2 | THR | 257 | 23.875 | 66.459 | 55.780 | 1.00 | 36.79 | DIC |
| ATOM | 2029 | C | THR | 257 | 26.900 | 65.089 | 57.717 | 1.00 | 35.26 | DIC |
| ATOM | 2030 | O | THR | 257 | 27.333 | 66.027 | 58.384 | 1.00 | 35.00 | DIC |
| ATOM | 2031 | N | ALA | 258 | 27.178 | 63.820 | 57.994 | 1.00 | 33.98 | DIC |
| ATOM | 2032 | CA | ALA | 258 | 28.038 | 63.457 | 59.111 | 1.00 | 32.05 | DIC |
| ATOM | 2033 | CB | ALA | 258 | 28.131 | 61.943 | 59.231 | 1.00 | 31.18 | DIC |
| ATOM | 2034 | C | ALA | 258 | 29.421 | 64.052 | 58.889 | 1.00 | 31.39 | DIC |
| ATOM | 2035 | O | ALA | 258 | 29.867 | 64.220 | 57.751 | 1.00 | 30.13 | DIC |
| ATOM | 2036 | N | TRP | 259 | 30.098 | 64.362 | 59.986 | 1.00 | 31.29 | DIC |
| ATOM | 2037 | CA | TRP | 259 | 31.429 | 64.955 | 59.937 | 1.00 | 29.92 | DIC |
| ATOM | 2038 | CB | TRP | 259 | 31.868 | 65.341 | 61.351 | 1.00 | 31.14 | DIC |
| ATOM | 2039 | CG | TRP | 259 | 33.230 | 65.947 | 61.400 | 1.00 | 31.00 | DIC |
| ATOM | 2040 | CD2 | TRP | 259 | 34.439 | 65.294 | 61.798 | 1.00 | 30.73 | DIC |
| ATOM | 2041 | CE2 | TRP | 259 | 35.484 | 66.235 | 61.668 | 1.00 | 30.83 | DIC |
| ATOM | 2042 | CE3 | TRP | 259 | 34.741 | 64.004 | 62.253 | 1.00 | 29.98 | DIC |
| ATOM | 2043 | CD1 | TRP | 259 | 33.576 | 67.221 | 61.053 | 1.00 | 30.91 | DIC |
| ATOM | 2044 | NE1 | TRP | 259 | 34.930 | 67.402 | 61.211 | 1.00 | 31.95 | DIC |
| ATOM | 2045 | CZ2 | TRP | 259 | 36.811 | 65.928 | 61.978 | 1.00 | 30.08 | DIC |
| ATOM | 2046 | CZ3 | TRP | 259 | 36.058 | 63.698 | 62.560 | 1.00 | 29.09 | DIC |
| ATOM | 2047 | CH2 | TRP | 259 | 37.077 | 64.657 | 62.422 | 1.00 | 30.00 | DIC |
| ATOM | 2048 | C | TRP | 259 | 32.486 | 64.043 | 59.313 | 1.00 | 29.26 | DIC |
| ATOM | 2049 | O | TRP | 259 | 32.509 | 62.831 | 59.558 | 1.00 | 27.60 | DIC |
| ATOM | 2050 | N | GLY | 260 | 33.360 | 64.645 | 58.508 | 1.00 | 27.51 | DIC |
| ATOM | 2051 | CA | GLY | 260 | 34.436 | 63.912 | 57.861 | 1.00 | 26.27 | DIC |
| ATOM | 2052 | C | GLY | 260 | 34.050 | 62.911 | 56.788 | 1.00 | 26.05 | DIC |
| ATOM | 2053 | O | GLY | 260 | 34.909 | 62.189 | 56.281 | 1.00 | 25.46 | DIC |
| ATOM | 2054 | N | ALA | 261 | 32.772 | 62.862 | 56.431 | 1.00 | 25.27 | DIC |
| ATOM | 2055 | CA | ALA | 261 | 32.307 | 61.922 | 55.417 | 1.00 | 25.33 | DIC |
| ATOM | 2056 | CB | ALA | 261 | 30.805 | 62.112 | 55.182 | 1.00 | 25.36 | DIC |
| ATOM | 2057 | C | ALA | 261 | 33.066 | 62.078 | 54.101 | 1.00 | 25.94 | DIC |
| ATOM | 2058 | O | ALA | 261 | 33.265 | 61.106 | 53.373 | 1.00 | 26.13 | DIC |
| ATOM | 2059 | N | ALA | 262 | 33.497 | 63.300 | 53.804 | 1.00 | 25.58 | DIC |
| ATOM | 2060 | CA | ALA | 262 | 34.215 | 63.585 | 52.564 | 1.00 | 26.27 | DIC |
| ATOM | 2061 | CB | ALA | 262 | 34.544 | 65.078 | 52.476 | 1.00 | 26.51 | DIC |
| ATOM | 2062 | C | ALA | 262 | 35.482 | 62.762 | 52.350 | 1.00 | 26.63 | DIC |
| ATOM | 2063 | O | ALA | 262 | 35.900 | 62.566 | 51.205 | 1.00 | 26.59 | DIC |
| ATOM | 2064 | N | ARG | 263 | 36.109 | 62.275 | 53.418 | 1.00 | 25.79 | DIC |
| ATOM | 2065 | CA | ARG | 263 | 37.310 | 61.487 | 53.194 | 1.00 | 25.44 | DIC |
| ATOM | 2066 | CB | ARG | 263 | 38.201 | 61.364 | 54.435 | 1.00 | 28.92 | DIC |
| ATOM | 2067 | CG | ARG | 263 | 39.579 | 60.821 | 54.018 | 1.00 | 33.60 | DIC |
| ATOM | 2068 | CD | ARG | 263 | 40.483 | 60.390 | 55.155 | 1.00 | 37.93 | DIC |
| ATOM | 2069 | NE | ARG | 263 | 41.350 | 61.455 | 55.663 | 1.00 | 40.10 | DIC |
| ATOM | 2070 | CZ | ARG | 263 | 40.995 | 62.342 | 56.588 | 1.00 | 41.46 | DIC |
| ATOM | 2071 | NH1 | ARG | 263 | 41.856 | 63.267 | 56.987 | 1.00 | 41.19 | DIC |
| ATOM | 2072 | NH2 | ARG | 263 | 39.780 | 62.304 | 57.116 | 1.00 | 43.96 | DIC |
| ATOM | 2073 | C | ARG | 263 | 36.988 | 60.092 | 52.687 | 1.00 | 22.70 | DIC |
| ATOM | 2074 | O | ARG | 263 | 37.883 | 59.374 | 52.248 | 1.00 | 20.54 | DIC |
| ATOM | 2075 | N | PHE | 264 | 35.722 | 59.691 | 52.757 | 1.00 | 20.27 | DIC |
| ATOM | 2076 | CA | PHE | 264 | 35.354 | 58.373 | 52.245 | 1.00 | 19.95 | DIC |
| ATOM | 2077 | CB | PHE | 264 | 33.894 | 58.031 | 52.559 | 1.00 | 19.65 | DIC |
| ATOM | 2078 | CG | PHE | 264 | 33.433 | 56.738 | 51.934 | 1.00 | 18.28 | DIC |
| ATOM | 2079 | CD1 | PHE | 264 | 33.634 | 55.522 | 52.582 | 1.00 | 17.29 | DIC |
| ATOM | 2080 | CD2 | PHE | 264 | 32.835 | 56.734 | 50.674 | 1.00 | 18.28 | DIC |
| ATOM | 2081 | CE1 | PHE | 264 | 33.248 | 54.321 | 51.986 | 1.00 | 18.07 | DIC |
| ATOM | 2082 | CE2 | PHE | 264 | 32.446 | 55.534 | 50.069 | 1.00 | 17.91 | DIC |
| ATOM | 2083 | CZ | PHE | 264 | 32.654 | 54.328 | 50.729 | 1.00 | 17.29 | DIC |
| ATOM | 2084 | C | PHE | 264 | 35.526 | 58.425 | 50.728 | 1.00 | 19.57 | DIC |
| ATOM | 2085 | O | PHE | 264 | 36.140 | 57.548 | 50.126 | 1.00 | 18.73 | DIC |
| ATOM | 2086 | N | THR | 265 | 34.976 | 59.474 | 50.124 | 1.00 | 20.91 | DIC |
| ATOM | 2087 | CA | THR | 265 | 35.045 | 59.665 | 48.680 | 1.00 | 22.89 | DIC |
| ATOM | 2088 | CB | THR | 265 | 34.289 | 60.932 | 48.252 | 1.00 | 24.07 | DIC |
| ATOM | 2089 | OG1 | THR | 265 | 32.974 | 60.922 | 48.820 | 1.00 | 27.93 | DIC |
| ATOM | 2090 | CG2 | THR | 265 | 34.179 | 60.989 | 46.734 | 1.00 | 25.93 | DIC |
| ATOM | 2091 | C | THR | 265 | 36.496 | 59.810 | 48.252 | 1.00 | 23.30 | DIC |
| ATOM | 2092 | O | THR | 265 | 36.932 | 59.238 | 47.251 | 1.00 | 23.08 | DIC |
| ATOM | 2093 | N | GLU | 266 | 37.243 | 60.583 | 49.026 | 1.00 | 23.53 | DIC |
| ATOM | 2094 | CA | GLU | 266 | 38.646 | 60.804 | 48.733 | 1.00 | 25.20 | DIC |
| ATOM | 2095 | CB | GLU | 266 | 39.256 | 61.723 | 49.784 | 1.00 | 28.98 | DIC |
| ATOM | 2096 | CG | GLU | 266 | 40.736 | 61.936 | 49.607 | 1.00 | 36.19 | DIC |
| ATOM | 2097 | CD | GLU | 266 | 41.240 | 63.096 | 50.427 | 1.00 | 39.75 | DIC |
| ATOM | 2098 | OE1 | GLU | 266 | 40.976 | 63.113 | 51.650 | 1.00 | 41.33 | DIC |
| ATOM | 2099 | OE2 | GLU | 266 | 41.896 | 63.991 | 49.846 | 1.00 | 42.17 | DIC |
| ATOM | 2100 | C | GLU | 266 | 39.408 | 59.482 | 48.687 | 1.00 | 23.71 | DIC |
| ATOM | 2101 | O | GLU | 266 | 40.195 | 59.251 | 47.778 | 1.00 | 22.46 | DIC |
| ATOM | 2102 | N | LEU | 267 | 39.173 | 58.616 | 49.670 | 1.00 | 22.23 | DIC |
| ATOM | 2103 | CA | LEU | 267 | 39.842 | 57.320 | 49.701 | 1.00 | 21.24 | DIC |
| ATOM | 2104 | CB | LEU | 267 | 39.601 | 56.617 | 51.044 | 1.00 | 21.75 | DIC |
| ATOM | 2105 | CG | LEU | 267 | 40.509 | 57.054 | 52.195 | 1.00 | 22.58 | DIC |
| ATOM | 2106 | CD1 | LEU | 267 | 39.979 | 56.545 | 53.520 | 1.00 | 23.20 | DIC |
| ATOM | 2107 | CD2 | LEU | 267 | 41.918 | 56.531 | 51.938 | 1.00 | 24.01 | DIC |
| ATOM | 2108 | C | LEU | 267 | 39.346 | 56.443 | 48.562 | 1.00 | 19.60 | DIC |
| ATOM | 2109 | O | LEU | 267 | 40.137 | 55.786 | 47.888 | 1.00 | 19.14 | DIC |
| ATOM | 2110 | N | ALA | 268 | 38.035 | 56.436 | 48.340 | 1.00 | 19.54 | DIC |
| ATOM | 2111 | CA | ALA | 268 | 37.458 | 55.630 | 47.271 | 1.00 | 21.17 | DIC |
| ATOM | 2112 | CB | ALA | 268 | 35.949 | 55.838 | 47.209 | 1.00 | 22.40 | DIC |
| ATOM | 2113 | C | ALA | 266 | 38.091 | 55.999 | 45.933 | 1.00 | 23.39 | DIC |
| ATOM | 2114 | O | ALA | 268 | 38.382 | 55.127 | 45.108 | 1.00 | 23.18 | DIC |
| ATOM | 2115 | N | GLY | 269 | 38.306 | 57.295 | 45.727 | 1.00 | 23.42 | DIC |
| ATOM | 2116 | CA | GLY | 269 | 38.896 | 57.759 | 44.486 | 1.00 | 25.69 | DIC |
| ATOM | 2117 | C | GLY | 269 | 40.364 | 57.408 | 44.346 | 1.00 | 27.18 | DIC |
| ATOM | 2118 | O | GLY | 269 | 40.919 | 57.479 | 43.249 | 1.00 | 27.61 | DIC |
| ATOM | 2119 | N | SER | 270 | 40.995 | 57.029 | 45.452 | 1.00 | 27.80 | DIC |
| ATOM | 2120 | CA | SER | 270 | 42.409 | 56.670 | 45.438 | 1.00 | 28.87 | DIC |
| ATOM | 2121 | CB | SER | 270 | 43.065 | 57.070 | 46.757 | 1.00 | 29.06 | DIC |
| ATOM | 2122 | OG | SER | 270 | 42.560 | 56.279 | 47.818 | 1.00 | 31.44 | DIC |
| ATOM | 2123 | C | SER | 270 | 42.616 | 55.174 | 45.221 | 1.00 | 28.53 | DIC |
| ATOM | 2124 | O | SER | 270 | 43.750 | 54.704 | 45.156 | 1.00 | 28.86 | DIC |
| ATOM | 2125 | N | LEU | 271 | 41.525 | 54.425 | 45.105 | 1.00 | 27.87 | DIC |
| ATOM | 2126 | CA | LEU | 271 | 41.633 | 52.981 | 44.925 | 1.00 | 28.38 | DIC |
| ATOM | 2127 | CB | LEU | 271 | 40.240 | 52.337 | 44.928 | 1.00 | 26.77 | DIC |
| ATOM | 2128 | CG | LEU | 271 | 39.478 | 52.415 | 46.256 | 1.00 | 25.46 | DIC |
| ATOM | 2129 | CD1 | LEU | 271 | 38.123 | 51.727 | 46.125 | 1.00 | 24.92 | DIC |
| ATOM | 2130 | CD2 | LEU | 271 | 40.310 | 51.757 | 47.348 | 1.00 | 24.78 | DIC |
| ATOM | 2131 | C | LEU | 271 | 42.383 | 52.579 | 43.658 | 1.00 | 29.08 | DIC |
| ATOM | 2132 | O | LEU | 271 | 42.146 | 53.122 | 42.583 | 1.00 | 27.21 | DIC |
| ATOM | 2133 | N | THR | 272 | 43.288 | 51.616 | 43.804 | 1.00 | 29.70 | DIC |
| ATOM | 2134 | CA | THR | 272 | 44.075 | 51.115 | 42.687 | 1.00 | 30.05 | DIC |
| ATOM | 2135 | CB | THR | 272 | 45.091 | 50.055 | 43.158 | 1.00 | 30.32 | DIC |
| ATOM | 2136 | OG1 | THR | 272 | 45.943 | 50.617 | 44.164 | 1.00 | 32.13 | DIC |
| ATOM | 2137 | CG2 | THR | 272 | 45.935 | 49.578 | 41.996 | 1.00 | 32.54 | DIC |
| ATOM | 2138 | C | THR | 272 | 43.159 | 50.471 | 41.649 | 1.00 | 29.64 | DIC |
| ATOM | 2139 | O | THR | 272 | 43.195 | 50.830 | 40.471 | 1.00 | 29.15 | DIC |
| ATOM | 2140 | N | THR | 273 | 42.344 | 49.520 | 42.103 | 1.00 | 28.68 | DIC |
| ATOM | 2141 | CA | THR | 273 | 41.418 | 48.803 | 41.236 | 1.00 | 28.77 | DIC |
| ATOM | 2142 | CB | THR | 273 | 41.707 | 47.281 | 41.225 | 1.00 | 31.01 | DIC |
| ATOM | 2143 | OG1 | THR | 273 | 43.095 | 47.050 | 40.955 | 1.00 | 33.16 | DIC |
| ATOM | 2144 | CG2 | THR | 273 | 40.868 | 46.593 | 40.153 | 1.00 | 33.04 | DIC |
| ATOM | 2145 | C | THR | 273 | 39.967 | 48.984 | 41.675 | 1.00 | 26.88 | DIC |
| ATOM | 2146 | O | THR | 273 | 39.642 | 48.858 | 42.854 | 1.00 | 25.29 | DIC |
| ATOM | 2147 | N | VAL | 274 | 39.095 | 49.273 | 40.717 | 1.00 | 24.92 | DIC |
| ATOM | 2148 | CA | VAL | 274 | 37.683 | 49.449 | 41.025 | 1.00 | 23.86 | DIC |
| ATOM | 2149 | CB | VAL | 274 | 37.276 | 50.932 | 41.006 | 1.00 | 23.31 | DIC |
| ATOM | 2150 | CG1 | VAL | 274 | 35.839 | 51.070 | 41.469 | 1.00 | 23.87 | DIC |
| ATOM | 2151 | CG2 | VAL | 274 | 38.191 | 51.737 | 41.906 | 1.00 | 24.43 | DIC |
| ATOM | 2152 | C | VAL | 274 | 36.819 | 48.700 | 40.019 | 1.00 | 23.07 | DIC |
| ATOM | 2153 | O | VAL | 274 | 36.593 | 49.176 | 38.902 | 1.00 | 23.08 | DIC |
| ATOM | 2154 | N | PRO | 275 | 36.330 | 47.510 | 40.400 | 1.00 | 21.69 | DIC |
| ATOM | 2155 | CD | PRO | 275 | 36.566 | 46.776 | 41.654 | 1.00 | 20.50 | DIC |
| ATOM | 2156 | CA | PRO | 275 | 35.488 | 46.726 | 39.497 | 1.00 | 21.63 | DIC |
| ATOM | 2157 | CB | PRO | 275 | 35.088 | 45.524 | 40.352 | 1.00 | 21.49 | DIC |
| ATOM | 2158 | CG | PRO | 275 | 36.254 | 45.356 | 41.253 | 1.00 | 21.48 | DIC |
| ATOM | 2159 | C | PRO | 275 | 34.273 | 47.522 | 39.048 | 1.00 | 21.99 | DIC |
| ATOM | 2160 | O | PRO | 275 | 33.736 | 48.338 | 39.805 | 1.00 | 21.78 | DIC |
| ATOM | 2161 | N | GLU | 276 | 33.852 | 47.284 | 37.810 | 1.00 | 23.31 | DIC |
| ATOM | 2162 | CA | GLU | 276 | 32.683 | 47.943 | 37.249 | 1.00 | 23.31 | DIC |
| ATOM | 2163 | CB | GLU | 276 | 32.297 | 47.256 | 35.934 | 1.00 | 23.72 | DIC |
| ATOM | 2164 | CG | GLU | 276 | 30.950 | 47.667 | 35.362 | 1.00 | 26.81 | DIC |
| ATOM | 2165 | CD | GLU | 276 | 30.875 | 49.144 | 35.067 | 1.00 | 27.36 | DIC |
| ATOM | 2166 | OE1 | GLU | 276 | 31.941 | 49.794 | 35.028 | 1.00 | 31.05 | DIC |
| ATOM | 2167 | OE2 | GLU | 276 | 29.755 | 49.656 | 34.868 | 1.00 | 29.72 | DIC |
| ATOM | 2168 | C | GLU | 276 | 31.525 | 47.846 | 38.244 | 1.00 | 23.02 | DIC |
| ATOM | 2169 | O | GLU | 276 | 30.797 | 48.811 | 38.473 | 1.00 | 23.54 | DIC |
| ATOM | 2170 | N | GLU | 277 | 31.376 | 46.678 | 38.854 | 1.00 | 23.75 | DIC |
| ATOM | 2171 | CA | GLU | 277 | 30.294 | 46.464 | 39.804 | 1.00 | 25.40 | DIC |
| ATOM | 2172 | CB | GLU | 277 | 30.168 | 44.970 | 40.120 | 1.00 | 29.16 | DIC |
| ATOM | 2173 | CG | GLU | 277 | 28.715 | 44.493 | 40.230 | 1.00 | 35.80 | DIC |
| ATOM | 2174 | CD | GLU | 277 | 28.022 | 44.308 | 38.873 | 1.00 | 37.33 | DIC |
| ATOM | 2175 | OE1 | GLU | 277 | 28.104 | 45.210 | 38.005 | 1.00 | 38.08 | DIC |
| ATOM | 2176 | OE2 | GLU | 277 | 27.378 | 43.251 | 38.685 | 1.00 | 39.35 | DIC |
| ATOM | 2177 | C | GLU | 277 | 30.407 | 47.275 | 41.104 | 1.00 | 24.54 | DIC |
| ATOM | 2178 | O | GLU | 277 | 29.484 | 47.275 | 41.914 | 1.00 | 23.39 | DIC |
| ATOM | 2179 | N | TRP | 278 | 31.531 | 47.962 | 41.300 | 1.00 | 23.06 | DIC |
| ATOM | 2180 | CA | TRP | 278 | 31.732 | 48.795 | 42.489 | 1.00 | 20.91 | DIC |
| ATOM | 2181 | CB | TRP | 278 | 33.181 | 48.700 | 42.986 | 1.00 | 18.69 | DIC |
| ATOM | 2182 | CG | TRP | 278 | 33.521 | 47.467 | 43.781 | 1.00 | 17.97 | DIC |
| ATOM | 2183 | CD2 | TRP | 278 | 34.676 | 47.279 | 44.613 | 1.00 | 16.79 | DIC |
| ATOM | 2184 | CE2 | TRP | 278 | 34.610 | 45.970 | 45.130 | 1.00 | 15.02 | DIC |
| ATOM | 2185 | CE3 | TRP | 278 | 35.763 | 48.095 | 44.970 | 1.00 | 16.53 | DIC |
| ATOM | 2186 | CD1 | TRP | 278 | 32.822 | 46.294 | 43.828 | 1.00 | 16.18 | DIC |
| ATOM | 2187 | NE1 | TRP | 278 | 33.470 | 45.390 | 44.636 | 1.00 | 16.36 | DIC |
| ATOM | 2188 | CZ2 | TRP | 278 | 35.589 | 45.450 | 45.989 | 1.00 | 14.14 | DIC |
| ATOM | 2189 | CZ3 | TRP | 278 | 36.742 | 47.577 | 45.828 | 1.00 | 14.30 | DIC |
| ATOM | 2190 | CH2 | TRP | 278 | 36.644 | 46.268 | 46.324 | 1.00 | 13.79 | DIC |
| ATOM | 2191 | C | TRP | 278 | 31.441 | 50.258 | 42.152 | 1.00 | 21.28 | DIC |
| ATOM | 2192 | O | TRP | 278 | 31.315 | 51.093 | 43.046 | 1.00 | 20.17 | DIC |
| ATOM | 2193 | N | ALA | 279 | 31.344 | 50.563 | 40.860 | 1.00 | 22.70 | DIC |
| ATOM | 2194 | CA | ALA | 279 | 31.102 | 51.935 | 40.412 | 1.00 | 24.93 | DIC |
| ATOM | 2195 | CB | ALA | 279 | 30.915 | 51.972 | 38.899 | 1.00 | 27.07 | DIC |
| ATOM | 2196 | C | ALA | 279 | 29.903 | 52.563 | 41.098 | 1.00 | 25.99 | DIC |
| ATOM | 2197 | O | ALA | 279 | 29.976 | 53.690 | 41.590 | 1.00 | 25.88 | DIC |
| ATOM | 2198 | N | GLY | 280 | 28.795 | 51.834 | 41.134 | 1.00 | 27.56 | DIC |
| ATOM | 2199 | CA | GLY | 280 | 27.608 | 52.372 | 41.772 | 1.00 | 30.18 | DIC |
| ATOM | 2200 | C | GLY | 280 | 27.801 | 52.629 | 43.257 | 1.00 | 31.52 | DIC |
| ATOM | 2201 | O | GLY | 280 | 27.427 | 53.680 | 43.776 | 1.00 | 32.79 | DIC |
| ATOM | 2202 | N | LYS | 281 | 28.413 | 51.668 | 43.937 | 1.00 | 31.54 | DIC |
| ATOM | 2203 | CA | LYS | 281 | 28.656 | 51.766 | 45.370 | 1.00 | 32.16 | DIC |
| ATOM | 2204 | CB | LYS | 281 | 29.079 | 50.396 | 45.911 | 1.00 | 31.67 | DIC |
| ATOM | 2205 | CG | LYS | 281 | 27.951 | 49.386 | 46.055 | 1.00 | 33.55 | DIC |
| ATOM | 2206 | CD | LYS | 281 | 28.490 | 48.084 | 46.637 | 1.00 | 34.90 | DIC |
| ATOM | 2207 | CE | LYS | 281 | 27.450 | 47.351 | 47.477 | 1.00 | 36.99 | DIC |
| ATOM | 2208 | NZ | LYS | 281 | 26.693 | 46.315 | 46.724 | 1.00 | 38.19 | DIC |
| ATOM | 2209 | C | LYS | 281 | 29.664 | 52.815 | 45.826 | 1.00 | 32.48 | DIC |
| ATOM | 2210 | O | LYS | 281 | 29.484 | 53.440 | 46.867 | 1.00 | 32.45 | DIC |
| ATOM | 2211 | N | LEU | 282 | 30.717 | 53.007 | 45.044 | 1.00 | 31.92 | DIC |
| ATOM | 2212 | CA | LEU | 282 | 31.758 | 53.953 | 45.403 | 1.00 | 33.81 | DIC |
| ATOM | 2213 | CB | LEU | 282 | 33.121 | 53.402 | 44.968 | 1.00 | 32.92 | DIC |
| ATOM | 2214 | CG | LEU | 282 | 34.051 | 52.818 | 46.044 | 1.00 | 34.56 | DIC |
| ATOM | 2215 | CD1 | LEU | 282 | 33.283 | 52.458 | 47.313 | 1.00 | 32.48 | DIC |
| ATOM | 2216 | CD2 | LEU | 282 | 34.752 | 51.605 | 45.466 | 1.00 | 32.62 | DIC |
| ATOM | 2217 | C | LEU | 282 | 31.599 | 55.383 | 44.893 | 1.00 | 35.67 | DIC |
| ATOM | 2218 | O | LEU | 282 | 32.284 | 56.281 | 45.373 | 1.00 | 37.01 | DIC |
| ATOM | 2219 | N | ALA | 283 | 30.711 | 55.605 | 43.932 | 1.00 | 37.96 | DIC |
| ATOM | 2220 | CA | ALA | 283 | 30.501 | 56.944 | 43.381 | 1.00 | 40.04 | DIC |
| ATOM | 2221 | CB | ALA | 283 | 30.102 | 57.925 | 44.489 | 1.00 | 39.26 | DIC |
| ATOM | 2222 | C | ALA | 283 | 31.718 | 57.476 | 42.611 | 1.00 | 41.65 | DIC |
| ATOM | 2223 | OT1 | ALA | 283 | 31.501 | 57.913 | 41.458 | 1.00 | 43.46 | DIC |
| ATOM | 2224 | OT2 | ALA | 283 | 32.858 | 57.460 | 43.139 | 1.00 | 41.61 | DIC |
| ATOM | 2225 | MN | MN | 400 | 29.002 | 50.705 | 62.676 | 1.00 | 11.41 | |
| ATOM | 2226 | C1 | LAT | 1347 | 21.881 | 53.893 | 65.661 | 1.00 | 28.17 | |
| ATOM | 2227 | C2 | LAT | 1347 | 22.143 | 52.528 | 65.080 | 1.00 | 26.76 | |
| ATOM | 2228 | C3 | LAT | 1347 | 22.196 | 51.524 | 66.241 | 1.00 | 26.19 | |
| ATOM | 2229 | C4 | LAT | 1347 | 23.382 | 51.940 | 67.197 | 1.00 | 25.62 | |
| ATOM | 2230 | C5 | LAT | 1347 | 23.232 | 53.243 | 67.815 | 1.00 | 25.09 | |
| ATOM | 2231 | C6 | LAT | 1347 | 24.216 | 53.701 | 68.651 | 1.00 | 23.05 | |
| ATOM | 2232 | O1 | LAT | 1347 | 21.713 | 55.161 | 65.005 | 1.00 | 29.10 | |
| ATOM | 2233 | O2 | LAT | 1347 | 21.103 | 52.228 | 64.162 | 1.00 | 26.57 | |
| ATOM | 2234 | O3 | LAT | 1347 | 22.437 | 50.258 | 65.682 | 1.00 | 23.28 | |
| ATOM | 2235 | O4 | LAT | 1347 | 24.642 | 52.079 | 66.577 | 1.00 | 24.26 | |
| ATOM | 2236 | O5 | LAT | 1347 | 22.951 | 54.232 | 66.677 | 1.00 | 26.25 | |
| ATOM | 2237 | O6 | LAT | 1347 | 24.608 | 52.886 | 69.776 | 1.00 | 26.54 | |
| ATOM | 2238 | C1′ | LAT | 1347 | 20.966 | 59.301 | 65.099 | 1.00 | 32.51 | |
| ATOM | 2239 | C2′ | LAT | 1347 | 22.189 | 58.806 | 65.964 | 1.00 | 31.92 | |
| ATOM | 2240 | C3′ | LAT | 1347 | 22.654 | 57.435 | 65.390 | 1.00 | 31.34 | |
| ATOM | 2241 | C4′ | LAT | 1347 | 21.441 | 56.495 | 65.526 | 1.00 | 31.39 | |
| ATOM | 2242 | C5′ | LAT | 1347 | 20.188 | 56.938 | 64.702 | 1.00 | 32.37 | |
| ATOM | 2243 | C6′ | LAT | 1347 | 18.954 | 56.016 | 64.828 | 1.00 | 33.23 | |
| ATOM | 2244 | O1′ | LAT | 1347 | 20.519 | 60.547 | 65.593 | 1.00 | 33.36 | |
| ATOM | 2245 | O2′ | LAT | 1347 | 23.244 | 59.759 | 65.873 | 1.00 | 29.82 | |
| ATOM | 2246 | O3′ | LAT | 1347 | 23.772 | 56.970 | 66.183 | 1.00 | 32.03 | |
| ATOM | 2247 | O5′ | LAT | 1347 | 19.866 | 58.294 | 65.174 | 1.00 | 31.84 | |
| ATOM | 2248 | O6′ | LAT | 1347 | 18.076 | 56.261 | 63.749 | 1.00 | 37.37 | |
| ATOM | 2249 | N1 | UPG | 341 | 27.322 | 44.456 | 57.775 | 1.00 | 12.21 | |
| ATOM | 2250 | C2 | UPG | 341 | 27.705 | 43.789 | 56.548 | 1.00 | 12.58 | |
| ATOM | 2251 | N3 | UPG | 341 | 26.796 | 44.078 | 55.491 | 1.00 | 13.15 | |
| ATOM | 2252 | C4 | UPG | 341 | 25.580 | 44.942 | 55.533 | 1.00 | 11.78 | |
| ATOM | 2253 | C5 | UPG | 341 | 25.244 | 45.587 | 56.772 | 1.00 | 12.67 | |
| ATOM | 2254 | C6 | UPG | 341 | 26.069 | 45.358 | 57.842 | 1.00 | 12.46 | |
| ATOM | 2255 | O2 | UPG | 341 | 28.671 | 43.078 | 56.445 | 1.00 | 11.36 | |
| ATOM | 2256 | O4 | UPG | 341 | 24.924 | 45.060 | 54.495 | 1.00 | 13.21 | |
| ATOM | 2257 | C4* | UPG | 341 | 28.139 | 45.820 | 61.018 | 1.00 | 13.58 | |
| ATOM | 2258 | O4* | UPG | 341 | 27.383 | 44.722 | 60.301 | 1.00 | 11.69 | |
| ATOM | 2259 | C3* | UPG | 341 | 28.971 | 46.440 | 59.908 | 1.00 | 10.52 | |
| ATOM | 2260 | O3* | UPG | 341 | 30.005 | 47.173 | 60.471 | 1.00 | 11.62 | |
| ATOM | 2261 | C2* | UPG | 341 | 29.272 | 45.220 | 59.097 | 1.00 | 11.03 | |
| ATOM | 2262 | O2* | UPG | 341 | 30.400 | 44.483 | 59.548 | 1.00 | 9.93 | |
| ATOM | 2263 | C1* | UPG | 341 | 28.051 | 44.379 | 59.135 | 1.00 | 12.83 | |
| ATOM | 2264 | C5* | UPG | 341 | 26.814 | 46.217 | 61.487 | 1.00 | 13.32 | |
| ATOM | 2265 | O5* | UPG | 341 | 26.265 | 47.573 | 61.238 | 1.00 | 16.42 | |
| ATOM | 2266 | PA | UPG | 341 | 26.748 | 49.072 | 61.108 | 1.00 | 14.28 | |
| ATOM | 2267 | O1A | UPG | 341 | 28.187 | 49.241 | 61.244 | 1.00 | 14.57 | |
| ATOM | 2268 | O2A | UPG | 341 | 26.213 | 49.674 | 59.838 | 1.00 | 16.15 | |
| ATOM | 2269 | O3A | UPG | 341 | 25.983 | 49.214 | 62.459 | 1.00 | 14.41 | |
| ATOM | 2270 | PB | UPG | 341 | 25.800 | 50.467 | 63.365 | 1.00 | 14.57 | |
| ATOM | 2271 | O1B | UPG | 341 | 27.120 | 51.044 | 63.507 | 1.00 | 12.20 | |
| ATOM | 2272 | O2B | UPG | 341 | 24.933 | 51.389 | 62.659 | 1.00 | 14.49 | |
| ATOM | 2273 | O3B | UPG | 341 | 24.918 | 50.295 | 64.666 | 1.00 | 17.88 | |
| ATOM | 2274 | C1′ | UPG | 341 | 25.388 | 49.351 | 65.853 | 1.00 | 24.44 | |
| ATOM | 2275 | C2′ | UPG | 341 | 26.854 | 49.185 | 66.369 | 1.00 | 27.13 | |
| ATOM | 2276 | C3′ | UPG | 341 | 27.779 | 48.150 | 65.490 | 1.00 | 26.87 | |
| ATOM | 2277 | C4′ | UPG | 341 | 27.069 | 46.698 | 65.546 | 1.00 | 27.54 | |
| ATOM | 2278 | C5′ | UPG | 341 | 25.562 | 47.005 | 64.928 | 1.00 | 27.76 | |
| ATOM | 2279 | C6′ | UPG | 341 | 24.646 | 45.802 | 64.906 | 1.00 | 29.63 | |
| ATOM | 2280 | F2′ | UPG | 341 | 27.412 | 50.500 | 66.415 | 1.00 | 26.09 | |
| ATOM | 2281 | O3′ | UPG | 341 | 29.067 | 48.093 | 66.053 | 1.00 | 26.84 | |
| ATOM | 2282 | O4′ | UPG | 341 | 26.972 | 46.296 | 66.918 | 1.00 | 26.57 | |
| ATOM | 2283 | O5′ | UPG | 341 | 24.748 | 48.031 | 65.729 | 1.00 | 26.37 | |
| ATOM | 2284 | O6′ | UPG | 341 | 23.367 | 45.385 | 65.355 | 1.00 | 28.54 | |
| ATOM | 2285 | O | HOH | 512 | 54.673 | 48.356 | 65.523 | 1.00 | 25.15 | |
| ATOM | 2286 | O | HOH | 513 | 57.867 | 51.960 | 56.478 | 1.00 | 18.24 | |
| ATOM | 2287 | O | HOH | 514 | 25.903 | 36.241 | 53.371 | 1.00 | 23.04 | |
| ATOM | 2288 | O | HOH | 515 | 43.782 | 40.049 | 44.203 | 1.00 | 19.20 | |
| ATOM | 2289 | O | HOH | 516 | 25.269 | 48.689 | 57.913 | 1.00 | 20.10 | |
| ATOM | 2290 | O | HOH | 517 | 33.923 | 31.711 | 71.928 | 1.00 | 18.55 | |
| ATOM | 2291 | O | HOH | 518 | 30.388 | 30.310 | 61.293 | 1.00 | 22.80 | |
| ATOM | 2292 | O | HOH | 519 | 43.430 | 56.956 | 57.195 | 1.00 | 15.05 | |
| ATOM | 2293 | O | HOH | 520 | 49.150 | 58.809 | 68.184 | 1.00 | 22.34 | |
| ATOM | 2294 | O | HOH | 521 | 46.243 | 43.905 | 76.655 | 1.00 | 17.00 | |
| ATOM | 2295 | O | HOH | 522 | 26.676 | 56.544 | 69.515 | 1.00 | 16.47 | |
| ATOM | 2296 | O | HOH | 523 | 30.000 | 48.381 | 81.295 | 1.00 | 22.90 | |
| ATOM | 2297 | O | HOH | 524 | 44.731 | 41.738 | 76.380 | 1.00 | 28.37 | |
| ATOM | 2298 | O | HOH | 525 | 43.888 | 30.365 | 73.504 | 1.00 | 30.03 | |
| ATOM | 2299 | O | HOH | 526 | 38.608 | 33.989 | 82.252 | 1.00 | 31.63 | |
| ATOM | 2300 | O | HOH | 527 | 45.486 | 58.476 | 72.471 | 1.00 | 17.02 | |
| ATOM | 2301 | O | HOH | 528 | 36.439 | 60.989 | 58.566 | 1.00 | 21.83 | |
| ATOM | 2302 | O | HOH | 529 | 22.558 | 57.605 | 60.909 | 1.00 | 22.34 | |
| ATOM | 2303 | O | HOH | 510 | 22.910 | 48.263 | 67.559 | 1.00 | 27.02 | |
| ATOM | 2304 | O | HOH | 342 | 33.837 | 51.323 | 71.040 | 1.00 | 10.52 | |
| ATOM | 2305 | O | HOH | 343 | 33.436 | 45.012 | 60.346 | 1.00 | 11.03 | |
| ATOM | 2306 | O | HOH | 344 | 41.858 | 32.593 | 61.292 | 1.00 | 14.48 | |
| ATOM | 2307 | O | HOH | 345 | 30.314 | 40.780 | 63.189 | 1.00 | 10.46 | |
| ATOM | 2308 | O | HOH | 346 | 26.633 | 38.049 | 51.120 | 1.00 | 13.67 | |
| ATOM | 2309 | O | HOH | 347 | 25.661 | 58.468 | 67.699 | 1.00 | 17.94 | |
| ATOM | 2310 | O | HOH | 348 | 26.776 | 40.465 | 85.536 | 1.00 | 21.27 | |
| ATOM | 2311 | O | HOH | 349 | 34.269 | 66.072 | 73.361 | 1.00 | 16.12 | |
| ATOM | 2312 | O | HOH | 350 | 42.055 | 55.697 | 75.118 | 1.00 | 15.08 | |
| ATOM | 2313 | O | HOH | 351 | 24.932 | 44.993 | 86.462 | 1.00 | 15.36 | |
| ATOM | 2314 | O | HOH | 352 | 32.634 | 57.485 | 71.956 | 1.00 | 7.12 | |
| ATOM | 2315 | O | HOH | 353 | 41.424 | 63.579 | 62.874 | 1.00 | 10.82 | |
| ATOM | 2316 | O | HOH | 354 | 34.311 | 34.079 | 70.575 | 1.00 | 12.06 | |
| ATOM | 2317 | O | HOH | 355 | 31.944 | 30.308 | 71.197 | 1.00 | 16.77 | |
| ATOM | 2318 | O | HOH | 356 | 31.522 | 46.216 | 65.787 | 1.00 | 15.52 | |
| ATOM | 2319 | O | HOH | 357 | 34.596 | 56.735 | 75.449 | 1.00 | 12.10 | |
| ATOM | 2320 | O | HOH | 358 | 20.938 | 53.671 | 79.071 | 1.00 | 14.85 | |
| ATOM | 2321 | O | HOH | 359 | 42.846 | 60.054 | 74.844 | 1.00 | 14.85 | |
| ATOM | 2322 | O | HOH | 360 | 33.043 | 62.643 | 72.666 | 1.00 | 13.35 | |
| ATOM | 2323 | O | HOH | 361 | 31.465 | 47.811 | 83.263 | 1.00 | 19.48 | |
| ATOM | 2324 | O | HOH | 362 | 17.810 | 43.979 | 69.451 | 1.00 | 13.43 | |
| ATOM | 2325 | O | HOH | 363 | 44.880 | 61.186 | 73.189 | 1.00 | 13.38 | |
| ATOM | 2326 | O | HOH | 364 | 52.815 | 48.765 | 69.102 | 1.00 | 13.97 | |
| ATOM | 2327 | O | HOH | 365 | 20.792 | 39.749 | 57.158 | 1.00 | 11.23 | |
| ATOM | 2328 | O | HOH | 366 | 38.954 | 52.211 | 81.847 | 1.00 | 16.78 | |
| ATOM | 2329 | O | HOH | 367 | 22.941 | 41.574 | 81.754 | 1.00 | 17.53 | |
| ATOM | 2330 | O | HOH | 368 | 39.526 | 27.671 | 66.454 | 1.00 | 19.67 | |
| ATOM | 2331 | O | HOH | 369 | 13.108 | 47.479 | 74.594 | 1.00 | 15.51 | |
| ATOM | 2332 | O | HOH | 370 | 19.447 | 44.093 | 61.807 | 1.00 | 16.50 | |
| ATOM | 2333 | O | HOH | 371 | 30.399 | 40.374 | 82.936 | 1.00 | 9.51 | |
| ATOM | 2334 | O | HOH | 372 | 19.157 | 32.432 | 71.731 | 1.00 | 21.71 | |
| ATOM | 2335 | O | HOH | 374 | 40.516 | 58.977 | 78.099 | 1.00 | 16.53 | |
| ATOM | 2336 | O | HOH | 375 | 25.173 | 34.444 | 79.349 | 1.00 | 15.37 | |
| ATOM | 2337 | O | HOH | 376 | 46.578 | 58.693 | 67.532 | 1.00 | 13.15 | |
| ATOM | 2338 | O | HOH | 377 | 14.471 | 52.264 | 70.498 | 1.00 | 18.62 | |
| ATOM | 2339 | O | HOH | 378 | 39.660 | 63.544 | 78.200 | 1.00 | 16.08 | |
| ATOM | 2340 | O | HOH | 379 | 55.295 | 50.486 | 64.035 | 1.00 | 16.60 | |
| ATOM | 2341 | O | HOH | 381 | 48.181 | 45.311 | 54.971 | 1.00 | 17.29 | |
| ATOM | 2342 | O | HOH | 382 | 43.505 | 31.236 | 76.092 | 1.00 | 19.33 | |
| ATOM | 2343 | O | HOH | 383 | 32.590 | 44.068 | 37.933 | 1.00 | 27.84 | |
| ATOM | 2344 | O | HOH | 384 | 37.969 | 40.917 | 41.166 | 1.00 | 21.67 | |
| ATOM | 2345 | O | HOH | 385 | 31.948 | 51.058 | 85.079 | 1.00 | 18.57 | |
| ATOM | 2346 | O | HOH | 386 | 28.224 | 60.122 | 88.185 | 1.00 | 23.70 | |
| ATOM | 2347 | O | HOH | 387 | 45.297 | 40.751 | 78.746 | 1.00 | 19.73 | |
| ATOM | 2348 | O | HOH | 389 | 18.713 | 51.902 | 66.002 | 1.00 | 16.80 | |
| ATOM | 2349 | O | HOH | 390 | 24.809 | 39.213 | 56.261 | 1.00 | 19.74 | |
| ATOM | 2350 | O | HOH | 391 | 38.859 | 65.613 | 79.857 | 1.00 | 21.35 | |
| ATOM | 2351 | O | HOH | 392 | 23.682 | 28.524 | 65.843 | 1.00 | 25.37 | |
| ATOM | 2352 | O | HOH | 393 | 30.619 | 45.293 | 47.694 | 1.00 | 19.83 | |
| ATOM | 2353 | O | HOH | 395 | 16.069 | 54.647 | 63.765 | 1.00 | 21.81 | |
| ATOM | 2354 | O | HOH | 396 | 26.626 | 51.713 | 89.753 | 1.00 | 22.40 | |
| ATOM | 2355 | O | HOH | 397 | 28.064 | 49.899 | 39.247 | 1.00 | 26.58 | |
| ATOM | 2356 | O | HOH | 398 | 47.650 | 50.646 | 79.240 | 1.00 | 25.75 | |
| ATOM | 2357 | O | HOH | 399 | 27.302 | 61.880 | 78.488 | 1.00 | 23.12 | |
| ATOM | 2358 | O | HOH | 401 | 52.702 | 53.348 | 61.415 | 1.00 | 15.81 | |
| ATOM | 2359 | O | HOH | 402 | 41.238 | 60.307 | 45.671 | 1.00 | 21.78 | |
| ATOM | 2360 | O | HOH | 403 | 48.221 | 59.205 | 72.644 | 1.00 | 20.33 | |
| ATOM | 2361 | O | HOH | 404 | 32.922 | 39.506 | 83.843 | 1.00 | 26.81 | |
| ATOM | 2362 | O | HOH | 405 | 33.647 | 51.875 | 54.608 | 1.00 | 26.78 | |
| ATOM | 2363 | O | HOH | 406 | 34.729 | 27.098 | 73.772 | 1.00 | 24.68 | |
| ATOM | 2364 | O | HOH | 407 | 47.014 | 45.510 | 42.850 | 1.00 | 28.16 | |
| ATOM | 2365 | O | HOH | 409 | 46.142 | 41.147 | 44.690 | 1.00 | 22.35 | |
| ATOM | 2366 | O | HOH | 410 | 12.827 | 43.035 | 64.396 | 1.00 | 17.06 | |
| ATOM | 2367 | O | HOH | 411 | 45.907 | 38.504 | 58.159 | 1.00 | 25.94 | |
| ATOM | 2368 | O | HOH | 414 | 29.975 | 27.632 | 68.843 | 1.00 | 21.59 | |
| ATOM | 2369 | O | HOH | 416 | 49.328 | 45.517 | 72.009 | 1.00 | 16.75 | |
| ATOM | 2370 | O | HOH | 417 | 37.408 | 65.908 | 57.617 | 1.00 | 27.94 | |
| ATOM | 2371 | O | HOH | 418 | 43.941 | 57.374 | 74.443 | 1.00 | 14.75 | |
| ATOM | 2372 | O | HOH | 419 | 29.018 | 46.583 | 79.445 | 1.00 | 10.62 | |
| ATOM | 2373 | O | HOH | 420 | 28.318 | 58.314 | 71.194 | 1.00 | 13.85 | |
| ATOM | 2374 | O | HOH | 421 | 17.267 | 52.333 | 63.327 | 1.00 | 23.92 | |
| ATOM | 2375 | O | HOH | 422 | 11.697 | 46.878 | 72.387 | 1.00 | 18.34 | |
| ATOM | 2376 | O | HOH | 423 | 25.498 | 40.253 | 50.998 | 1.00 | 13.87 | |
| ATOM | 2377 | O | HOH | 424 | 17.533 | 50.046 | 67.514 | 1.00 | 16.25 | |
| ATOM | 2378 | O | HOH | 425 | 14.824 | 50.534 | 68.599 | 1.00 | 21.93 | |
| ATOM | 2379 | O | HOH | 426 | 45.832 | 63.034 | 71.218 | 1.00 | 26.79 | |
| ATOM | 2380 | O | HOH | 429 | 54.376 | 51.863 | 65.883 | 1.00 | 18.45 | |
| ATOM | 2381 | O | HOH | 430 | 50.977 | 47.216 | 76.379 | 1.00 | 23.22 | |
| ATOM | 2382 | O | HOH | 431 | 42.874 | 53.348 | 76.065 | 1.00 | 19.36 | |
| ATOM | 2383 | O | HOH | 432 | 43.449 | 34.666 | 60.899 | 1.00 | 17.96 | |
| ATOM | 2384 | O | HOH | 433 | 34.130 | 67.571 | 75.561 | 1.00 | 24.13 | |
| ATOM | 2385 | O | HOH | 434 | 27.453 | 42.471 | 87.224 | 1.00 | 32.46 | |
| ATOM | 2386 | O | HOH | 435 | 52.158 | 48.653 | 66.492 | 1.00 | 20.64 | |
| ATOM | 2387 | O | HOH | 436 | 34.404 | 47.778 | 83.422 | 1.00 | 19.34 | |
| ATOM | 2388 | O | HOH | 437 | 25.914 | 58.255 | 60.777 | 1.00 | 23.62 | |
| ATOM | 2389 | O | HOH | 438 | 24.526 | 68.909 | 75.214 | 1.00 | 33.24 | |
| ATOM | 2390 | O | HOH | 440 | 28.786 | 26.819 | 61.937 | 1.00 | 25.45 | |
| ATOM | 2391 | O | HOH | 441 | 41.960 | 34.523 | 52.538 | 1.00 | 19.68 | |
| ATOM | 2392 | O | HOH | 442 | 53.720 | 46.358 | 69.854 | 1.00 | 21.67 | |
| ATOM | 2393 | O | HOH | 445 | 28.804 | 38.524 | 84.456 | 1.00 | 25.60 | |
| ATOM | 2394 | O | HOH | 446 | 43.669 | 62.480 | 59.056 | 1.00 | 24.50 | |
| ATOM | 2395 | O | HOH | 447 | 13.951 | 50.958 | 82.440 | 1.00 | 34.99 | |
| ATOM | 2396 | O | HOH | 448 | 36.623 | 67.353 | 73.056 | 1.00 | 18.09 | |
| ATOM | 2397 | O | HOH | 449 | 23.895 | 37.020 | 54.976 | 1.00 | 23.23 | |
| ATOM | 2398 | O | HOH | 450 | 31.251 | 53.568 | 87.928 | 1.00 | 24.41 | |
| ATOM | 2399 | O | HOH | 452 | 21.273 | 33.232 | 57.364 | 1.00 | 24.12 | |
| ATOM | 2400 | O | HOH | 453 | 30.982 | 60.462 | 63.192 | 1.00 | 31.37 | |
| ATOM | 2401 | O | HOH | 454 | 45.825 | 39.919 | 74.932 | 1.00 | 33.02 | |
| ATOM | 2402 | O | HOH | 458 | 47.378 | 51.451 | 51.491 | 1.00 | 28.58 | |
| ATOM | 2403 | O | HOH | 459 | 42.709 | 47.306 | 81.851 | 1.00 | 31.79 | |
| ATOM | 2404 | O | HOH | 461 | 31.207 | 27.302 | 65.372 | 1.00 | 22.14 | |
| ATOM | 2405 | O | HOH | 462 | 28.964 | 26.675 | 71.181 | 1.00 | 28.72 | |
| ATOM | 2406 | O | HOH | 463 | 39.686 | 64.266 | 81.983 | 1.00 | 31.45 | |
| ATOM | 2407 | O | HOH | 466 | 42.215 | 44.109 | 85.084 | 1.00 | 39.24 | |
| ATOM | 2408 | O | HOH | 469 | 43.225 | 37.756 | 80.665 | 1.00 | 31.08 | |
| ATOM | 2409 | O | HOH | 477 | 33.002 | 24.938 | 70.787 | 1.00 | 44.27 | |
| ATOM | 2410 | O | HOH | 497 | 47.753 | 62.512 | 67.276 | 1.00 | 31.26 | |
| ATOM | 2411 | O | HOH | 611 | 14.151 | 39.876 | 64.132 | 1.00 | 26.85 | |
| ATOM | 2412 | O | HOH | 612 | 41.690 | 31.049 | 59.011 | 1.00 | 25.32 | |
| ATOM | 2413 | O | HOH | 613 | 52.238 | 45.010 | 68.097 | 1.00 | 25.29 | |
| ATOM | 2414 | O | HOH | 614 | 41.963 | 62.794 | 78.957 | 1.00 | 25.34 | |
| ATOM | 2415 | O | HOH | 615 | 18.316 | 43.721 | 58.649 | 1.00 | 23.51 | |
| ATOM | 2416 | O | HOH | 616 | 30.695 | 46.612 | 87.680 | 1.00 | 32.98 | |
| ATOM | 2417 | O | HOH | 617 | 24.606 | 40.630 | 48.428 | 1.00 | 28.20 | |
| ATOM | 2418 | O | HOH | 618 | 41.654 | 52.764 | 78.344 | 1.00 | 27.63 | |
| ATOM | 2419 | O | HOH | 620 | 45.215 | 49.791 | 80.776 | 1.00 | 31.54 | |
| ATOM | 2420 | O | HOH | 621 | 33.859 | 53.902 | 56.156 | 1.00 | 28.15 | |
| ATOM | 2421 | O | HOH | 622 | 36.203 | 37.715 | 83.838 | 1.00 | 31.32 | |
| ATOM | 2422 | O | HOH | 623 | 20.511 | 54.964 | 81.434 | 1.00 | 30.95 | |
| ATOM | 2423 | O | HOH | 624 | 44.439 | 51.283 | 46.458 | 1.00 | 27.81 | |
| ATOM | 2424 | O | HOH | 625 | 26.468 | 26.377 | 71.132 | 1.00 | 27.97 | |
| ATOM | 2425 | O | HOH | 626 | 41.327 | 54.925 | 84.791 | 1.00 | 35.97 | |
| ATOM | 2426 | O | HOH | 627 | 50.663 | 46.031 | 56.545 | 1.00 | 33.15 | |
| ATOM | 2427 | O | HOH | 628 | 49.607 | 45.005 | 75.531 | 1.00 | 29.21 | |
| ATOM | 2428 | O | HOH | 530 | 49.701 | 50.415 | 80.874 | 1.00 | 34.92 | |
| ATOM | 2429 | O | HOH | 531 | 29.995 | 55.495 | 89.602 | 1.00 | 30.40 | |
| ATOM | 2430 | O | HOH | 532 | 18.278 | 55.827 | 85.115 | 1.00 | 29.80 | |
| ATOM | 2431 | O | HOH | 533 | 34.321 | 52.394 | 85.636 | 1.00 | 31.13 | |
| ATOM | 2432 | O | HOH | 534 | 17.335 | 58.634 | 66.816 | 1.00 | 32.18 | |
| ATOM | 2433 | O | HOH | 535 | 37.008 | 41.209 | 84.114 | 1.00 | 33.40 | |
| ATOM | 2434 | O | HOH | 536 | 22.018 | 35.145 | 55.681 | 1.00 | 33.42 | |
| ATOM | 2435 | O | HOH | 537 | 23.707 | 41.537 | 52.919 | 1.00 | 35.31 | |
| ATOM | 2436 | O | HOH | 538 | 21.046 | 31.384 | 65.966 | 1.00 | 29.47 | |
| ATOM | 2437 | O | HOH | 543 | 20.341 | 47.928 | 87.042 | 1.00 | 29.97 | |
| ATOM | 2438 | O | HOH | 545 | 37.912 | 28.687 | 70.597 | 1.00 | 29.70 | |
| ATOM | 2439 | O | HOH | 546 | 22.366 | 34.826 | 80.821 | 1.00 | 30.63 | |
| ATOM | 2440 | O | HOH | 547 | 47.995 | 43.524 | 52.725 | 1.00 | 39.01 | |
| ATOM | 2441 | O | HOH | 548 | 41.270 | 50.998 | 82.378 | 1.00 | 34.24 | |
| ATOM | 2442 | O | HOH | 550 | 14.319 | 46.589 | 53.840 | 1.00 | 33.32 | |
| ATOM | 2443 | O | HOH | 552 | 34.230 | 42.579 | 42.029 | 1.00 | 29.38 | |
| ATOM | 2444 | O | HOH | 554 | 26.628 | 36.503 | 48.890 | 1.00 | 28.95 | |
| ATOM | 2445 | O | HOH | 557 | 25.845 | 65.038 | 64.799 | 1.00 | 31.69 | |
| ATOM | 2446 | O | HOH | 560 | 13.335 | 52.360 | 50.917 | 1.00 | 34.81 | |
| ATOM | 2447 | O | HOH | 561 | 24.566 | 50.832 | 53.862 | 1.00 | 29.06 | |
| ATOM | 2448 | O | HOH | 562 | 15.990 | 59.624 | 77.366 | 1.00 | 35.62 | |
| ATOM | 2449 | O | HOH | 563 | 36.875 | 51.110 | 84.792 | 1.00 | 36.48 | |
| ATOM | 2450 | O | HOH | 565 | 33.575 | 33.855 | 50.824 | 1.00 | 33.97 | |
| ATOM | 2451 | O | HOH | 570 | 22.705 | 37.189 | 50.422 | 1.00 | 34.04 | |
| ATOM | 2452 | O | HOH | 582 | 33.723 | 54.774 | 87.078 | 1.00 | 20.22 | |
| ATOM | 2453 | O | HOH | 583 | 31.373 | 44.992 | 62.477 | 1.00 | 31.80 | |
| ATOM | 2454 | O | HOH | 584 | 23.792 | 53.593 | 49.409 | 1.00 | 35.49 | |
| END | | | | | | | | | | |

List of References

1. Tzeng, Y. L. & Stephens, D. S. Epidemiology and pathogenesis of Neisseria meningitidis. Microbes and Infection 2, 687-700 (2000).

2. Wakarchuk, W. W., Martin, A., Jennings, M. P., Moxon, E. R. & Richards, J. C. Functional relationships of the genetic locus encoding the glycosyltransferase enzymes involved in expression of the lacto-N-neotetraose terminal lipopolysac-charide structure in Neisseria meningitidis. J. Biol. Chem. 271, 19166-19173 (1996).

3. Moran, A. P., Prendergast, M. M. & Appelmelk, B. J. Molecular mimicry of host structures by bacterial lipopolysaccharides and its contribution to disease. FEMS Immunol. Med. Microbiol. 16, 105-115 (1996).

4. Kahler, C. & Stephens, D. Genetic basis for biosynthesis, structure and function of meningococcal Lipooligosaccharide (endotoxin). Crit. Rev. Microbiol. 24, 281-334 (1998).

5. Campbell, J. A., Davies, G. J., Bulone, V. & Henrissat, B. A classification of nucleotide-diphospho-sugar glycosyltransferase based on amino acid sequence similarities. Biochem. J. 329, 929-939 (1997).

6. Wakarchuk, W., Cunningham, A., Watson, D. & Young, M. Role of paired basic residues in the expression of active recombinant galactosyltansferases from bacterial pathogen Neisseria meningitidis. Prot. Engineering 11, 295-302. (1998).

7. Whitfield, C. & Roberts, I. S. Structure, assembly and regulation of expression of capsules in Escherichia coli. Mol. Microbiol. 31, 1307-1319 (1999).

8. Takayama, S. et al. Selective inhibition of b-1,4 and a-1,3-galactosyltransferases: donor sugar-nucleotide based approach. Bioorg. Med. Chem. 7, 401-409 (1999).

9. Sinnott, M. L. Catalytic Mechanisms of Enzymatic Glycosyl Transfer. Chem. Rev. 90, 1171-1202 (1990).

10. Davies, G., Withers, S. G. & Sinnott M. L. in Comprehensive Biological Catalysis, Vol. 1 (ed.

Sinnott, M. L.) 119-208 (Academic Press, London, 1997).

11. Zechel, D. L. & Withers, S. G. Glycosidase mechanisms: anatomy of a finely tuned catalyst Acc. Chem. Res. 33, 11-18 (2000).

12. Vrielink, A., Ruger, W., Driessen, H. P. C. & Freemont P. S. Crystal structure of the DNA modifying enzyme b-glucosyltransferase in the presence and absence of the substrate uridine diphosphoglucose. EMBO J. 13, 3413-3422 (1994).

13. Charnock, S. & Davies, G. Structure of the Nucleotide-Diphospho-Sugar transferase, SpSA from Bacillus subtilis, in native and Nucleotide-Complexed Forms. Biochemistry 38, 6380-6385 (1998).

14. Gastinel, L., Cambillau, C. & Bourne, Y. Crystal structures of the bovine b4galactosyltransferse catalytic domain and its complex with uridine diphosphogalactose. EMBO J. 18, 3546-3557 (1999).

15. Ha, S., Walker, D., Shi, Y. & S., W. The 1.9 Å crystal structure of Escherichia coli MurG, a membrane-associated glycosyltransferase involved in peptidoglycan biosynthesis. Protein Sci. 9, 1045-1052 (2000).

16. Pedersen, L. C. et al. Heparan/Chondroitin Sulfate Biosynthesis: Structure and mechanism of human glucuronyltransferase I. J. Biol. Chem. 275, 34580-34585 (2000).

17. Ünligil, U. M. et al. X-ray crystal structure of rabbit N-acetylglucosaminyltransferase I; catalytic mechanism and a new protein superfamily. EMBO J. 19, 5269-5280 (2000).

18. Lu, G. TOP: a new method for protein structure comparison and similarity searches. J. Appl. Crystallogr. 33, 176-183 (2000).

19. Burkart, M. D. et al. Chemo-enzymatic synthesis of fluorinated sugar nucleotide: useful mechanistic probes for glycosyl transferases. Bioorg. Med. Chem. 8, 1937-1946 (2000).

20. Thoden, J. B. & Holden, H. M. Dramatic differences in the binding of UDP-galactose and UDP-glucose to UDP-galactose 4-epimerase from Escherichia coli. Biochemistry 37, 11469-11477 (1998).

21. Martin, J. L., Johnson, L. N. & Withers, S. G. Comparison of the binding of glucose and glucose 1-phosphate derivatives to T-state glycogen phosphorylase b. Biochemistry 29, 10745-10757 (1990).

22. Uitdehaag, J. C. M. et al. Catalysis in the a-amylase family: X-ray structures along the reaction pathway of cyclodextrin glycosyltransferase. Nature Struct Biol. 6,432436 (1999).

23. Brayer, G. D. et al. Subsite mapping of the human pancreatic a-amylase active site through structural, kinetic, and mutagenesis techniques. Biochemistry 39, 4778-4791 (2000).

24. Breton, C., Bettler, E., Joziasse, D. H., Geremia, R. A. & Imberty, A. Sequence-function relationships of prokaryotic and eukaryotic galactosyltransferases. J. Biochem. 123, 1000-1009 (1998).

25. Kapitonov, D. & Yu, R. K. Conserved domains of glycosyltransferases. Glycobiology 9, 961-978 (1999).

26. Busch, C. et al. A common motif of eukaryotic glycosyltransferases is essential for the enzymatic activity of large Clostridial cystotoxins. J. Biol. Chem. 273, 19566-19572 (1998).

27. Shibayamam, K., Ohsuka, S., Toshihiko, T., Yoshickika, A. & Ohta, M. Conserved structural regions involved in the catalytic mechanism of Escherichia coli K-12 WaaO (Rfal). J. Bacteriol. 180, 5313-5318 (1998).

28. Wiggins, C. A. R. & Munro, S. Activity of the yeast MNN1 a-1,3-mannosyltransferase requires a motif conserved in many other families of glycosyltransferases. Proc. Natl. Acad. Sci. USA 95, 7945-7950 (1998).

29. Hagen, F. K., Hazes, B., Raffo, R., deSa, D. & Tabak, L. A. Structure-function analysis of the UDPN-acetyl-D-galactosamine: polypeptide N-acetylgalactosaminyltransferase. J. Biol. Chem. 274, 6797-6803 (1999).

30. Moloney, D. J. et al. Fringe is a glycosyltransferase that modifies Notch. Nature 406, 369-375 (2000).

31. Brackner, K., Perez, L., Clausen, H. & Cohen, S. Glycosyltransferase activity of Fringe modulates Notch-Delta interactions. Nature 406, 411-415 (2000).

32. Lougheed, B. M. Sc., University of British Columbia (1998).

33. Glusker, J. Structural aspects of metal liganding to functional groups in proteins. Adv. Protein Chem. 42, 1-76 (1991).

34. Collaborative Computational Project Number 4. The CCP4 suite: Programs for protein crystallography. Acta Crystallogr. D 50, 760-763 (1994).

35. Burgi, H., Dunitz, J. & Shefter, E. Nucleophilic addition to a carbonyl group. J. Am. Chem. Soc. 95, 5065-5067 (1973).

36. Mitchell, E. P. et al. Ternary complex crystal structures of glycogen phosphorylase with the transition state analogue nojirimycin tetrazole and phosphate in the T and R states. Biochemistry 35, 7341-7355 (1996).

37. Watson, K. A. et al. Phosphorylase recognition and phosphorolysis of its oligosaccharide substrate: answers to a long outstanding question. EMBO J. 18, 4619-4632 (1999).

38. Artymuik, P. J., Rice, D. W., Poirrette, A. R & Willett, P. b-Glucosyltransferase and phophorylase reveal their common theme. Nature Struct. Biol. 2, 117-120 (1995).

39. Holm, L. & Sander, C. Evolutionary linlc between glycogen phosphorylase and a DNA modifying enzyme. EMBO J. 14, 1287-1293 (1995).

40. Ly, H. D. & Withers, S. G. Mutagenesis of glycosidases. Annu. Rev. Biochem. 68,487-522 (1999).

41. Van Duyne, G. D., Standaert, R. F., Karplus, P. A., Schreiber, S. L. & Clardy, J. Atomic structures of the human immunophilin FKBP-12 complexes with FK506 and rapamycin. J. Mol. Biol. 229, 105-124 (1993).

42. Gosselin, S., Alhussaini, M., Streiff, M. B., Takabayashi, K. & Palcic, M. M. A continuous spectrophotometric assay for glycosyltransferases. Anal. Biochem. 220, 92-97 (1994).

43. Leatherbarrow, R. Grafit Version 3.0. (Erithacus Software Ltd., Staines, UK., 1990).

44. Otwinowski, Z. & Minor, W. Processing of X-ray diffraction data collected in oscillation mode. Methods Enzymol. 276, 307-326 (1997).

45. Terwilliger, T. C. & Berendzen, J. Automated structure solution for MIR and MAD. Acta Crystallogr. D 55, 849-861 (1999).

46. Cowtan, K. DM: An automated procedure for phase improvement by density modification. Joint CCP4 and ESF-EACBM Newsletter on Protein Crystallography 31, 34-38 (1994).

47. McRee, D. E. Practical protein crystallography. A visual protein crystallographic software system for X11/XView. J. Mol. Graphics 10,44-46 (1992).

48. Brünger, A. et al. Crystallography and NMR system: A new software suit for macromolecular structure determination. Acta Crystallogr. D 54, 905-921 (1998).

49. Engh, R. & Huber, R. Accurate bond and angle parameter for X-ray protein structure refinement Acta Crystallogr. A 47, 392-400 (1991).

50. Kleywegt, G. & Jones, T. Model building and refinement practice. Methods Enzymol. 277, 208-230 (1997).

51. Laskowski, R., MacArthur, M., Moss, D. & Thornton, J. PROCHECK: a program to check the stereochemical quality of protein structures. J. Appl. CrystaUogr. 26, 283-291 (1993).

52. Kraulis, P. MOLSCRIPT: A program to produce both detailed and schematic plots of protein structures. J. Appl. Crystallogr. 24, 946-950 (1991).

53. Merritt, E. A. & Bacon, D. J. Raster3D: Photorealistic Molecular Graphics. Methods Enzymol. 277, 505-524 (1997).

54. Nichols, A., Sharp, K. & Honig, B. Protein folding association: insights from the interfacial and thermodynamic properties of hydrocarbons. Proteins 11(1991).

1. A crystal comprising a ligand binding pocket of a retaining glycosyltransferase enzyme.

2. A crystal as claimed in claim 1 wherein the ligand binding pocket is capable of associating with a donor molecule or analogue thereof, or an acceptor molecule or analogue thereof.

3. A crystal as claimed in claim 1 wherein the ligand binding pocket is capable of associating with a diphosphate group of a donor molecule, a nucleotide of a donor molecule, a heterocyclic base of a donor molecule, a sugar of a nucleotide of a donor molecule, a selected sugar of a donor molecule that is transferred to an acceptor, or an acceptor.

4. A crystal comprising a ligand binding pocket of a glycosyltransferase and a donor molecule or analogue thereof, from which it is possible to derive structural data for the donor molecule.

5. A crystal comprising the ligand binding pocket of a glycosyltransferase and an acceptor molecule or analogue thereof, from which it is possible to derive structural data for the acceptor molecule.

6. A crystal as claimed in claim 1 wherein the ligand binding pocket is defined by one or more amino acid residues of a glycosyltransferase of atomic interactions shown in Table 3.

7. A crystal as claimed in claim 1 defined by the structural coordinates of one or more atomic contacts or atomic interactions as shown in Table 3.

8. A crystal according to any preceding claim, wherein the glycosyltransferase enzyme is capable of catalysing a step in the biosynthesis of a lipooligosaccharide.

9. A crystal according to any preceding claim wherein the glycosyltransferase enzyme is derivable from a pathogenic microorganism.

10. A crystal according to claim 11, wherein the glycosyltransferase enzyme is derivable from a gram negative mucosal pathogen such as one selected from the group consisting of: Neisseria, Escherichia, Salmonella, Haemophilus, Moraxella, Bordatella, and Campylobacter.

11. A crystal according to any preceding claim, wherein the glycosyltransferase enzyme is a galactosyltransferase.

12. A crystal according to claim 7, wherein the galactosyltransferase is α-1,4-galactosyltransferase derivable from Neisseria meningitidis.

13. A crystal according to any preceding claim wherein the crystal comprises a glycosyltransferase enzyme having a mutation in the part of the enzyme which is involved in attachment to bacterial membranes.

14. A crystal according to any preceding claim wherein the crystal comprises a glycosyltransferase enzyme having a mutation in one or more cysteine residues.

15. A crystal according to any preceding claim, wherein the ligand binding pocket is complexed with a donor molecule or analogue thereof

16. A crystal according to claim 15, wherein the ligand binding pocket is complexed with UDP-2FGal.

17. A crystal according to claim 15 or 16, wherein the donor molecule or analogue thereof interacts with a loop comprising residues 75-80 and a loop comprising residues 246-251 of the glycosyltansferase enzyme.

18. A crystal according to any preceding claim wherein UDP-Gal is capable of acting as a donor molecule for the glycosyltransferase enzyme, and wherein the ligand binding pocket comprises at least one of the residues involved in binding to the UDP portion of UDP-Gal, namely: Asp 8, Ala 6, Ile 104, Lys 250, Gly 247 and His 78.

19. A crystal according to any preceding claim wherein the ligand binding pocket comprises at least one of the residues involved in shielding the reactive center C1′ atom from water, namely: Ile 76, Asp 103, Asp 153, Ala 154, Gly 155, Tyr 186, Gln 189, His 244, Cys 246 and Gly 247.

20. A crystal according to any preceding claim, wherein the ligand binding pocket comprises Gln 189.

21. A crystal according to any preceding claim wherein UDP-Gal is capable of acting as a donor molecule for the glycosyltransferase enzyme, and wherein the ligand binding pocket comprises at least one of the residues involved in binding to the galactosyl moiety of UDP-Gal, namely: Asp103, Arg 86, and Asp 188.

22. A crystal according to any preceding claim wherein the ligand binding pocket comprises at least one DXD motif.

23. A crystal according to any preceding claim wherein the ligand binding pocket is in association with a metal cofactor.

24. A crystal according to claim 23, wherein the metal cofactor is manganese.

25. A crystal according to claim 24, which comprises a Mn²⁺ ion co-ordinated with the side chain atoms of His 244, Asp 103 and Asp 105.

26. A crystal according to any preceding claim, wherein the ligand binding pocket is complexed with a acceptor molecule or analogue thereof.

27. A crystal according to claim 26, wherein the ligand binding pocket is complexed with 4-deoxylactose.

28. A crystal according to claim 26 or 27, wherein lactose is capable of acting as an acceptor molecule for the glycosyltransferase enzyme, and wherein the ligand binding pocket comprises at least one of the residues involved in binding to lactose, namely: Asp130, Gln 189, Val 76, His 78, Tyr 186, Cys 246, Gly 247, Phe 132, Pro 211, Pro 248, Thr 212 and Cys 246.

29. A crystal according to any preceding claim having the structural coordinates shown in Table 4 , 5, or 6.

30. A model of a ligand binding pocket of a glycosyltransferase enzyme made using a crystal according to any preceding claim.

31. A crystal of a retaining glycosyltransferase comprising the structural coordinates shown in Table 4, 5, or 6.

32. A model of a retaining glycosyltransferase made using a crystal according to claim 31.

33. A computer-readable medium having stored thereon a crystal or model according to any preceding claim.

34. A method of determining the secondary, tertiary, and/or quanternary structures of a polypeptide comprising the step of using a crystal or model according to any of the preceding claims.

35. A method of screening for a ligand capable of binding to a ligand binding pocket and/or modulating the function of a retaining glycosyltransferase, comprising the use of a crystal or model according to any preceding claim.

36. A ligand identified by a method according to claim 35.

37. A ligand according to claim 36 which is capable of inhibiting lipooligosaccharide biosynthesis.

38. A ligand according to claim 36 or 37 which is capable of causing oxidation of Cys 246.

39. A method for identifying a potential modulator of a glycosyltransferase by determining binding interactions between a test compound and atomic contacts of a model of a ligand binding pocket of a glycosyltransferase as claimed in any preceding claim comprising:
(a) generating the atomic contacts on a computer screen;
(b) generating test compounds with their spatial structure on the computer screen;
(c) determining whether the compounds associate or interact with the atomic contacts defining the glycosyltransferase;
(d) identifying test compounds that are potential modulators by their ability to enter into a selected number of atomic contacts.

40. A method for identifying a potential modulator of a glycosyltransferase function by docking a computer representation of a test compound with a computer representation of a model of a glycosyltransferase or a ligand binding pocket as claimed in any preceding claim.

41. A method for the design of ligands for a retaining glycosyltransferase based on the crystal or model of a donor molecule or portion thereof or an acceptor or portion thereof comprising using the structural coordinates shown in Table 4, 5, or 6.

42. A method as claimed in claim 41 comprising (a) generating a computer representation of a donor molecule or an acceptor molecule defined by the structural coordinates shown in Table 4, 5, or 6; (b) searching for molecules in a data base that are similar to the defined donor molecule or acceptor, using a searching computer program, or replacing portions of the donor molecule or acceptor molecule with similar chemical structures from a database using a compound building computer program.

43. A modulator of a glycosyltransferase comprising a donor molecule or an acceptor molecule having the shape and structure of a donor molecule or acceptor molecule in the active site binding pocket of a reaction catalyzed by a glycosyltransferase.

44. Use of a ligand or a modulator as claimed in any preceding claim in the manufacture of a medicament to treat and/or prevent a disease in a mammalian patient

45. A pharmaceutical composition comprising a ligand or a modulator according to any preceding claim and optionally a pharmaceutically acceptable carrier, diluent, excipient or adjuvant or any combination thereof.

46. A method of treating and/or preventing a disease comprising administering a ligand according to any preceding claim, and/or a pharmaceutical composition according to claim 45 to a mammalian patient.

47. A computer for producing a model or three-dimensional representation of a molecule or molecular complex, wherein said molecule or molecular complex comprises a retaining glycosyltransferase or ligand binding pocket thereof defined by structural coordinates of a retaining glycosyltransferase amino acids or a ligand binding pocket thereof, or comprises structural coordinates of atoms of a ligand or a three-dimensional representation of a homolog of said molecule or molecular complex, wherein said computer comprises:
(a) a machine-readable data storage medium comprising a data storage material encoded with machine readable data wherein said data comprises the structural coordinates of glycosyltransferase amino acids according to Table 4, 5, or 6 or a ligand binding pocket or a ligand thereof;
(b) a working memory for storing instructions for processing said machine-readable data;
(c) a central-processing unit coupled to said working memory and to said machine-readable data storage medium for processing said machine readable data into said three-dimensional representation; and
(d) a display coupled to said central-processing unit for displaying said three-dimensional representation.

48. A method of conducting a drug discovery business comprising:
(a) providing one or more systems or methods for identifying modulators based on a model according to any preceding claim;
(b) conducting therapeutic profiling of modulators identified in step (a), or further analogs thereof, for efficacy and toxicity in animals; and
(c) formulating a pharmaceutical composition including one or more agents identified in step (b) as having an acceptable therapeutic profile.

49. A method as claimed in claim 48 including establishing a distribution system for distributing the pharmaceutical composition for sale, and optionally establishing a sales group for marketing the pharmaceutical composition.

50. A method of conducting a target discovery business comprising:
(a) providing one or more system or method for identifying modulators based on a model as claimed in any preceding claim;
(b) optionally conducting therapeutic profiling of modulators identified in (a) for efficacy and toxicity in animals; and
(c) licensing to a third party the rights for further drug development and/or sales for agents identified in step (a), or analogs thereof.

51. A crystal comprising a glycosyltransferase ligand binding pocket, substantially as described herein and with reference to the accompanying figures.


SEQUENCE LISTING (7 sequences)

SEQ ID NO: 1 - protein, 310 aa, N. meningitidis
MDIVFAADDNYAAYLCVAAKSVEAAHPDTEIRFHVLDAGISEANRAAVAANLRGGGNIRFIDVNPEDFAGFPLNIRHISITTYARLKLGEYIADCDKVLYLDIDVLVRDSLKPLWDTDLGDNWLGACIDLFVERQNAYKQKIGMADGEYYFNAGVLLINLKKWRQHDIFKMACEWVEQYKDVMQYQDQDILNGLFKGGVCYANSRFNFMPTNDAFMANRFASRHTDPLYRDRTYTAMPVAVSHYCGPAKPWHRDCTAWGAERFTELAGSLTSVPEEWRGKLAVPHRVFPTKRMLQRWRRKLSARFLRKIY

SEQ ID NO: 2 - protein, 256 aa, N. gonorrhoeae
MDIVFAADDNYAAYLCVAAKSVEAAHPDTEIRFHVLDAGISEENRAAVAANLRGGGNIRFIDVNPEDFAGSPLNIRHISITTYARLKLGEYIADCDKVLYLDTDVLVRDGLKPLWDTDLGGNWVGACIDLFVERQEGYKQKIGMADGGVCYANSRFNFMPTNYAFMANGFASRHTDPLYLDRTNTAMPVAVSHYCGSAKPWHRDCTVWGAERFTELAGSLTTVPEEWRGKLAVPPTKCMLQRWRKKLSARFLRKIY

SEQ ID NO: 3 - protein, 302 aa, P. multocia
MNILFVSDDVYAKHLVVAIKSIINHNEKGISFYIFDLGIKDENKRNINDIVSSYGSEVNFIAVNEKEFESFPVQISYISLATYARLKAAEYLPDNLNKIIYLDVDVLVFNSLEMLWNVDVNNFLTAACYDSFIENEKSEHKKSISMSDKEYYFNAGVMLPNLDEWRKMDVFSRALQLLAMYPNQMIYQDQDILNILFRNKVCYLDCRFNFMPNQLERIKQYHKGKLSNLHSLEKTTMPVVISHYCGPEKAWHADCKHFNVYFYQKILAEITRGTDKERVLSIKTYLKALIRRIRYKFKYQVY

SEQ ID NO: 4 - protein, 330 aa, H. influenzae
MTDRQTDRQTDRQTDRQTDRQTDRQTDRQTDGRTVSQTMNIIFSSDHYYAPYLAVSIFSIIKHTPKKINFYILDMKINQENKTIINNLASAYSCKVFFLPVCESDFQNFPKTIDYISLATYARLNLTKYIKNIDKAIYIDVDTLTNSSLQELWNIDITNYYLAACRDTFIDVKNEAWKKTIGLEGYSYFNAGILLINLNKWKEENIFQKSINWMNKYNNVMKYQDQDILNGICKGKVKFINNRFNFTPTDRDLIKKKNLLCVKMPIVISHYCGPNKFWHKKCSHLNCHIGNLLLKEMDKIIDIPSSWYDHFEKIPFLIKIKRLRKRIKDH

SEQ ID NO: 5 - protein, 338 aa, E. coli
MSAHYFNPQEMINKTIIFDERPAASVASSFHVAYGIDKNFLFGCGVSITSVLLHNNDVSFVFHVFIDDIPEADIQRLAQLAKSYRTCIQIHLVNCERLKALPTTKNWSIAMYFRFVIADYFIDQQDKILYLDADIACQGNLKPLITMDLANNVAAVVTERDANWWSLRGQSLQCNELEKGYFNSGVLLINTLAWAQESVSAKAMEMLADKAIVSRLTYMDQDILNLILLGKVKFIDAKYNTQFSLYNELKKSFVCPINDETVIIHYVGPTKPWHYWHEYPSAQPFIKAKEASPWKNEPLMRPVNSNYARYCAKHNFKQNKPINGIMNYIYYFYLKIIK

SEQ ID NO: 6 - protein, 337 aa, S. typhimurium
MSRKYFEEEVIQQTLDYNYAQHSDADKFNIAYGIDKNFLFGCGVSIASVLLANPEKALAFHVFTDFFDEEDQQRFEALAKQWATQIVVYLIDCERLKSLPSTKNWTYATYFRFIIADYSDKTDREVLYLDADIACKGSLQELIDLNFAENEIAAVVAEGELEWWTKRSVSLATPGLVSGYFNAGFILINIPLWTAENISKKAIEMLKDPQVVQRITHLDQDVLNIFLVNKARFVDKKFNTQFSLNYELKDSVINPVDAETVFVHYIGPTKPWHSWGAYPVSQYFLQAKSNSPWSHCALLNPVTSHQLRYAAKHMFNQKHWTSGINYYIAYIKRKLLE

SEQ ID NO: 7 - protein, 371 aa, H. pyroll
MSIIIPIVIAFDNHYAMPAGVSLYSMLACAKTEHPQSQNDSEKLFYKIHCLVDNLSLENQSKLKETLAPFSAFSSLEFLDISTPNLHATPIEPSAIDKINEAFLQLNIYAKTRFSKMVMCRLFLASLFPDYDKIIMFDADTLFLNDVSESFFIPLDGYYFGAKDFASDKSPKHFQIVREKDPRQAFSLYEHYLNESDMQIIYESNYNAGFLVVNLKLWRADHLEERLLNLTHQKGQCVFYPEQDLLTLACYQKVLILPYIYNTHPFMANQKRFIPDKKEIVMLHFYFVGKPWVLPTFSYSKEWHETLLKTPFYAEYSVKFLKQMTECLSLKDKQKTFEFLAPLLNKKTLLEYVFFRLNRIFKRIKEKFFNS